(19) United States
(12) Patent Application Publication
(10) Pub. No.: US 2023/0235309 A1
(43) Pub. Date: Jul. 27, 2023

(54) ADENINE BASE EDITORS AND USES THEREOF

(71) Applicants: The Broad Institute, Inc., Cambridge, MA (US); President and Fellows of Harvard College, Cambridge, MA (US)

(72) Inventors: David R. Liu, Cambridge, MA (US); Michelle Richter, Cambridge, MA (US); Kevin Tianmeng Zhao, Cambridge, MA (US); Benjamin Thuronyi, Cambridge, MA (US); Gregory Newby, Cambridge, MA (US)

(73) Assignees: The Broad Institute, Inc., Cambridge, MA (US); President and Fellows of Harvard College, Cambridge, MA (US)

(21) Appl. No.: 17/797,701

(22) PCT Filed: Feb. 5, 2021
(86) PCT No.: PCT/US2021/016827

§ 371 Date: Aug. 4, 2022

Related U.S. Application Data
(60) Provisional application No. 62/970,666, filed on Feb. 5, 2020.

(51) Int. Cl. C12N 9/78; C12N 15/10

(52) U.S. Cl. CPC C12N 9/78; C12Y 305/04004; C12N 15/102; C07K 2319/80; C07K 2319/09; C07K 2319/92

(57) ABSTRACT

The present disclosure provides adenine base editors (ABEs) that are variants of known adenine base editors. The adenosine deaminase domain of a known ABE was modified to produce adenosine deaminase variants. The deaminase variants provided herein have broader compatibility with diverse napDNAbp domains, such as Cas homologs, for base editing applications. The ABEs provided herein comprise a deaminase variant and a napDNAbp domain. The ABEs provided herein exhibit reduced off-target editing effects while retaining high on-target editing efficiencies. These ABEs exhibit reduced off-target DNA editing effects and reduced off-target editing effects in cellular mRNA. In addition, methods for targeted nucleic acid editing are provided. Further provided are pharmaceutical compositions comprising the ABEs. Also provided are vectors and kits useful for the generation and delivery of the ABEs, including vector systems for engineering the ABEs through directed evolution. Cells containing such vectors and ABEs are also provided. Further provided are methods of treatment comprising administering the ABEs.

### RELATED APPLICATIONS

This application is a national stage filing under 35 U.S.C. § 371 of International PCT Application PCT/US2021/016827, filed Feb. 5, 2021, which claims priority under 35 U.S.C. § 119(e) to U.S. Provisional Application, U.S. Ser. No. 62/970,666, filed on Feb. 5, 2020, each of which is incorporated herein by reference.

### GOVERNMENT SUPPORT

This invention was made with government support under grant numbers U01 AI142756, RM1 HG009490, R01 EB022376, and R35 GM118062 awarded by the National Institutes of Health. The government has certain rights in the invention.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Jul. 27, 2022, is named B119570089US01-SEQ-EPG and is 1,654,749 bytes in size.

### BACKGROUND OF THE INVENTION

Base editors enable the precise installation of targeted point mutations in genomic DNA without creating double-stranded DNA breaks (DSBs). Adenine base editors (ABEs) convert a target A•T base pair to a G•C base pair. Because the mutation of G•C base pairs to A•T base pairs is the most common form of de novo mutation, ABEs have the potential to correct almost half of the known human pathogenic point mutations. The original adenine base editor, ABE7.10, can perform remarkably clean and efficient A•T-to-G•C conversion in DNA with very low levels of undesirable by-products, such as small insertions or deletions (indels), in cultured cells, adult mice, plants, and other organisms. Reference is made to International Patent Application No. PCT/US2018/056146, filed Oct. 16, 2018, Koblan et al., Nat Biotechnol (2018) and Gaudelli et al., Nature 551, 464-471 (2017).

Although adenine base editors (ABEs) in principle can correct the largest class of pathogenic point mutations, efforts to apply ABEs have been constrained by the limited compatibility of the adenosine deaminase domain of the ABEs with Cas homologs other than canonical Cas proteins, such as S. pyogenes Cas9 (SpCas9).

There is a need in the art for novel adenine base editors that have adenosine deaminase domains with broader compatibility with other Cas domains and also preferably have reduced off-target effects while retaining high on-target deamination efficiencies.

### SUMMARY OF THE INVENTION

The recent development of adenine base editors by fusion of an adenosine deaminase to Cas9 enables guide RNA (gRNA)-targeted single nucleotide deamination for A:T to G:C base pair conversion using adenine base editors within a specific target window. Various engineered base editors with improved DNA editing efficiencies have been recently developed. Reference is made to Komor, A. C. et al., Improved base excision repair inhibition and bacteriophage Mu Gam protein yields C:G-to-T:A base editors with higher efficiency and product purity, Sci Adv 3 (2017); Rees, H. A. et al., Improving the DNA specificity and applicability of base editing through protein engineering and protein delivery, Nat. Commun. 8, 15790 (2017); U.S. Patent Publication No. 2018/0073012, published Mar. 15, 2018, U.S. Patent Publication No. 2017/0121693, published May 4, 2017, International Publication No. WO 2017/070633, published Apr. 27, 2017, and U.S. Patent Publication No. 2015/0166980, published Jun. 18, 2015, U.S. Pat. No. 9,840,699, issued Dec. 12, 2017, U.S. Pat. No. 10,077,453, issued Sep. 18, 2018; International Application No. PCT/US2020/21362, filed Mar. 6, 2020; International Publication No. WO 2020/214842, published Oct. 22, 2020; International Application No. PCT/US2019/61685, filed Nov. 15, 2019, which published as WO 2020/102659 on May 22, 2020; and International Application No. PCT/US2020/062428, filed Nov. 25, 2020, each of which are incorporated herein in their entireties. Base editors (BEs) are typically fusions of a Cas (“CRISPR-associated”) domain and a nucleobase (or “base”) modification domain (e.g., a natural or evolved deaminase, such as an adenosine deaminase domain). In some cases, base editors may also include proteins or domains that alter cellular DNA repair processes to increase the efficiency, incorporation, and/or stability of the resulting single-nucleotide change.

Base editors reported to date contain a catalytically impaired Cas9 domain fused to a nucleobase modification domain. The Cas9 domain directs the nucleobase modification domain to directly convert one base to another at a guide RNA-programmed target site¹⁻³. Two classes of base editors have been developed to date^{1, 2, 14-16}: Cytosine base editors (CBEs), which convert C•G to T•A, and adenine base editors (ABEs), which convert A•T to G•C. Collectively, CBEs and ABEs enable the correction of all four types of transition mutations (C to T, G to A, A to G, and T to C). As half of known disease-associated gene variants are point mutations, and transition mutations account for ˜60% of known pathogenic point mutations^{4, 5}, base editors are being widely used to study and treat genetic diseases in a variety of cell types and organisms, including animal models of human genetic diseases^{3, 17-27}.

ABEs are especially useful for the study and correction of pathogenic alleles, as nearly half of pathogenic point mutations in principle can be corrected by converting an A•T base pair to a G•C base pair^{4, 5}. Many of the ABEs reported to date include a single polypeptide chain containing a heterodimer of a wild-type E. coli TadA monomer that plays a structural role during base editing and a laboratory-evolved E. coli TadA monomer (TadA*) that catalyzes deoxyadenosine deamination, and a Cas9 (D10A) nickase. Wild type E. coli TadA acts as a homodimer to deaminate an adenosine located in a tRNA anticodon loop, generating inosine (I). Although early ABE variants required a heterodimeric TadA containing an N-terminal wild-type TadA monomer for maximal activity, Joung et al. showed that later ABE variants have comparable activity with and without the wild-type TadA monomer⁴².

Base editing requires the presence of a PAM located approximately 15 base pairs from the target nucleotide(s) for canonical (i.e., S. pyogenes Cas9-derived) base editors¹⁻³. Each programmable DNA-binding protein domain recognizes a different PAM sequence. Only about one quarter of pathogenic transition point mutations have a suitably located canonical PAM “NGG” sequence that is compatible with S. pyogenes Cas9-derived base editors⁶⁻⁸. Cytidine deaminases have proven to be broadly compatible with many Cas homologs, including SaCas9⁸, SaCas9-KKH⁸, Cas12a (Cpf1)^{9, 10}, SpCas9-NG¹¹, and circularly permuted CP-Cas9s⁷, greatly expanding their targeting scope. Existing adenosine deaminases have unfortunately shown limited compatibility with Cas homologs. Some Cas homologs, such as SaCas9⁷, SaCas9-KKH⁷, SpCas9-NG¹¹, and CP-Cas9s⁷, are compatible with ABEs, but editing efficiencies are substantially lower than those of the corresponding CBEs, and other homologs such as LbCas12a and enAsCas12a show virtually no activity as an ABE^{9, 10}.

The present disclosure is based, at least in part, on the evolution of existing adenine base editors to produce variant ABEs with broadened compatibility with diverse Cas homologs, as well as improved targeting scope and efficiency. It was hypothesized that very low to modest efficiency of many non-SpCas9 ABEs arises from a low rate of adenosine deamination (low kₐₚₚ) combined with the shorter residence time on DNA (high k_{off}) of many Cas homologs compared to that of SpCas9²⁸. The adenosine deaminase domain of the ABE7.10 base editor is TadA7.10, a deoxyadenosine deaminase that was previously evolved from an E. coli tRNA adenosine deaminase (ecTadA, or TadA) to act on single-stranded DNA². TadA7.10 is also the deaminase domain of ABEmax, which is a variant of ABE7.10 that has been codon optimized for expression in human cells. TadA7.10 comprises the following substitutions in ecTadA: W23R, H36L, P48A, R51L, L84F, A106V, D108N, H123Y, S146C, D147Y, R152P, E155V, I156F, and K157N. Reference is made to International Publication No. WO 2018/027078, published Aug. 2, 2018; International Publication No. WO 2019/079347 published Apr. 25, 2019; International Publication No. WO 2019/226953, published Nov. 28, 2019; U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2020/214842, published Oct. 22, 2020, and International Patent Application No. PCT/US2020/033873, filed May 20, 2020, the contents of each of which are incorporated herein by reference in their entireties.

To address the above-described deficiency in the art, a new phage-assisted continuous evolution (PACE) ABE selection system was developed and applied to enhance the compatibility of the deaminase domains of ABEs with other Cas homologs, such as newly developed variants of Cas9 derived from S. pyogenes, newly developed variants of Cas9 derived from S. aureus, those derived from Lachnospiraceae bacterium, such as LbCas12a, those derived from Acidaminococcus sp. BV3L6, such as AsCas12a, and those derived from Streptococcus macacae NCTC 11558, such as SmacCas9 and Spy-macCas9. The adenosine deaminases disclosed herein are compatible with a variety of Cas homologs, including small-sized, circularly permuted, and evolved Cas homologs. The adenosine deaminases disclosed herein are compatible with a variety of Cas variants or homologs that include, but are not limited to, SaCas9, SaCas9-KKH, LbCas12a, enAsCas12a, SpCas9-NG, and circularly permuted variants CP1028-SpCas9 and CP1041-SpCas9. The adenosine deaminases disclosed herein are compatible with a combination of Cas variants, such as Cas9-NG-CP1041 and Cas9-NG-VRQR.

PACE and phage-assisted non-continuous evolution (PANCE) were applied to the adenosine deaminase domain of ABE7.10 to enhance base editing activity. The variants evolved from these experiments exhibit broader Cas homolog compatibility than ABE7.10. TadA-8e, an exemplary variant emerging from these PACE and PANCE experiments, contains eight (8) additional substitutions relative to TadA7.10. These eight mutations are A109S, T111R, D119N, H122N, Y147D, F149Y, T1661, and D167N in the TadA7.10 sequence of SEQ ID NO: 315. ABE8e, the adenine base editor that comprises TadA-8e, shows increased editing efficiency relative to ABE7.10, while maintaining on-target:off-target editing ratios of ABE7.10. For instance, an on-target:off-target editing ratio of about 1300:1 was observed at the human EMX1 (homeobox protein) gene target, and an on-target:off-target editing ratio of about 90:1 was observed at the clinically relevant human HBG target, which is associated with sickle cell disease. The PACE-evolved ABE variants disclosed herein, including ABE8e, expand the targeting scope, editing efficiency, and overall utility of adenine base editors. ABE8e also has increased processivity compared to ABE7.10, which makes it useful for applications such as screening, disrupting regulatory regions, and multiplexed base editing.

Accordingly, in some aspects, the disclosure provides an adenosine deaminase comprising an amino acid sequence that comprises substitutions selected from T111X₁, D119X₂, and F149X₃, relative to the amino acid sequence of SEQ ID NO: 315, wherein X₁ is any amino acid other than T, X₂ is any amino acid other than D, and X₃ is any amino acid other than F. The adenosine deaminases may further comprise at least one substitution selected from R26X₄, V88X₅, A109X₆, H122X₇, T166X₈, and D167X₉, relative to the amino acid sequence of SEQ ID NO: 315, wherein X₄ is any amino acid other than R, X₅ is any amino acid other than V, X₆ is any amino acid other than A, X₇ is any amino acid other than H, X₈ is any amino acid other than T, and X₉ is any amino acid other than D. In some aspects, the disclosure provides adenosine deaminase that further comprises a Y167X₁₀ relative to the amino acid sequence of SEQ ID NO: 315, wherein X₁₀ is any amino acid other than Y. In particular embodiments, X₁₀ is D. In some aspects, the adenosine deaminases of the disclosure may further comprise at least one substitution selected from K20X₁₁, R21X₁₂, V82X₁₃, and V106X₁₃ relative to the amino acid sequence of SEQ ID NO: 315, wherein XII is any amino acid other than K, X₁₂ is any amino acid other than R, X₁₃ is any amino acid other than V, and X₁₄ is any amino acid other than V.

In some aspects, the present disclosure provides adenosine deaminases that comprise T111R, D119N, and F149Y substitutions, and further comprises at least one substitution selected from R26C, V88A, A109S, H122N, T1661, and D167N, in the amino acid sequence corresponding to TadA7.10 (SEQ ID NO: 315). In some embodiments, the disclosed adenosine deaminase comprises the substitutions A109S, T111R, D119N, H122N, Y147D, F149Y, T1661 and D167N in its amino acid sequence. In certain embodiments, the adenosine deaminases comprise the substitutions V106W, A109S, T111R, D119N, H122N, Y147D, F149Y, T1661, and D167N. In an exemplary embodiment, the adenosine deaminase is TadA-8e (SEQ ID NO: 433). In another exemplary embodiment, the adenosine deaminase is TadA-8e (V106W) (SEQ ID NO: 434). In exemplary embodiments, any of the disclosed adenosine deaminases is used in a base editor, as further provided herein. In some embodiments, the disclosed adenosine deaminases exhibit higher editing efficiencies when paired in a base editor with certain Cas9 variants, such as circularly permuted variants CP1041 and CP1028, than exhibited by the TadA7.10 deaminase.

In some aspects, the present disclosure provides adenine base editors comprising a nucleic acid programmable DNA binding protein (napDNAbp) domain and any of the adenosine deaminases disclosed herein. In some aspects, the napDNAbp domain comprises a Cas9 protein or variant thereof. In some aspects, the napDNAbp domain comprise a nickase (e.g., an nCas9).

In some embodiments, the base editor comprises a napDNAbp domain and an adenosine deaminase that comprises the substitutions T111R, D119N, and F149Y, and further comprises at least one substitution selected from the group consisting of R26C, V88A, A109S, H122N, T1661, and D167N, in the amino acid sequence corresponding to TadA7.10 (SEQ ID NO: 315). In some embodiments, the base editor comprises an adenosine deaminase that comprises the substitutions A109S, T111R, D119N, H122N, Y147D, F149Y, T1661, and D167N. In some embodiments, the base editor comprises an adenosine deaminase that comprises at least one substitution selected from the group consisting of K20A, R21A, V82G, and V106W. In exemplary embodiments, the base editor comprises ABE8e. In exemplary embodiments, the base editor comprises ABE8e-NRCH.

The adenine base editors (ABEs) provided herein may be capable of maintaining DNA editing efficiency, and in some embodiments demonstrate improved DNA editing efficiencies, relative to existing adenine base editors, such as ABE7.10. In some embodiments, the ABEs described herein exhibit reduced off-target editing effects while retaining high on-target editing efficiencies. In certain embodiments, the disclosed ABEs exhibit reduced Cas9-independent off-target editing effects while retaining high on-target editing efficiencies. In certain embodiments, the disclosed ABEs exhibit reduced off-target editing effects in RNA, such as cellular mRNA.

In some embodiments, the adenine base editors provided herein are capable of limiting formation of indels in a DNA substrate. In some embodiments, the ABEs provided herein have an expanded target window for editing a DNA substrate than canonical ABEs (e.g., a target window that corresponds to protospacer positions 4-11, 8-14, or 9-14 of the target sequence, wherein protospacer position 0 corresponds to a position relative to the transcription start site of the target gene).

In some aspects, the present disclosure provides compositions comprising the adenine base editors as described herein and one or more guide RNAs, e.g., a single-guide RNA (“sgRNA”). In addition, the disclosure provides for nucleic acid molecules encoding and/or expressing the adenine base editors as described herein, as well as expression vectors or constructs for expressing the adenine base editors described herein and a gRNA, host cells comprising said nucleic acid molecules and expression vectors, and one or more gRNAs, and compositions for delivering and/or administering nucleic acid-based embodiments described herein.

The present disclosure further provides complexes comprising the adenine base editors described herein and a gRNA associated with the Cas9 domain of the base editor, such as a single guide RNA. The guide RNA may be 15-100 nucleotides in length and comprise a sequence of at least 10, at least 15, or at least 20 contiguous nucleotides that is complementary to a target nucleotide sequence.

The present disclosure further provides methods of DNA editing that make use of the adenine base editors disclosed herein. These methods may induce (or yield, provide, or cause) an actual or average deamination frequency of 80%, 82%, 84%, 86%, 88%, 90%, 92.5%, 95%, or 98% when contacted with a DNA comprising a target sequence selected from the group consisting of AAA, AAT, AAC, AAG, TAA, TAT, TAC, TAG, CAA, CAT, CAC, CAG, GAA, GAT, GAC, and GAG. In some embodiments, these editing methods induce (yield, provide, or cause) an average deamination frequency of at least 75%, at least 80%, at least 84%, or at least 86%.

The disclosed methods of DNA editing may involve multiplexed base editing using any of the disclosed ABEs. Such methods may involve transducing (e.g., via transfection) cells with a plurality of complexes each comprising a base editor (e.g., an ABE comprising a Cas9 nickase (nCas9) domain and an adenosine deaminase domain) and a gRNA.

In certain embodiments of the disclosed methods, a nucleic acid construct (e.g., a plasmid or an mRNA construct) that encodes the ABE is transfected into the cell separately from the nucleic acid construct that encodes the gRNA molecule. In certain embodiments, these components are encoded on a single construct and transfected together. Accordingly, in some aspects, the disclosure provides polynucleotides and vectors encoding any of the adenine base editors, or domains thereof, described herein. The nucleic acid sequences may be codon-optimized for expression in the cells of any organism of interest (e.g., a human). In certain embodiments, the nucleic acid sequence is codon-optimized for expression in human cells.

In other embodiments, the methods disclosed herein involve the introduction into cells of a complex comprising an ABE and a gRNA molecule that has been expressed and cloned outside of these cells. In other embodiments, the methods disclosed herein involve the introduction into cells of an ABE and a gRNA using a lipid or polymer (such as a cationic lipid or cationic polymer).

It should be appreciated that any fusion protein, e.g., any of the base editors described herein, may be introduced into the cell in any suitable way, either stably or transiently. In some embodiments, a base editor may be transfected into the cell. In some embodiments, the cell may be transduced or transfected with a nucleic acid construct that encodes a base editor. For example, a cell may be transduced (e.g., with a virus encoding a base editor) with a nucleic acid that encodes a base editor, or the translated base editor. As an additional example, a cell may be transfected (e.g., with a plasmid encoding a base editor) with a nucleic acid that encodes a base editor or the translated base editor. Such transductions or transfections may be stable or transient. In some embodiments, cells expressing a base editor or containing a base editor may be transduced or transfected with one or more gRNA molecules, for example. In some embodiments, a plasmid expressing a base editor may be introduced into cells through electroporation (e.g., using an ATX MaxCyte electroporator), transient transfection (e.g., lipofection), stable genome integration (e.g., piggybac), viral transduction, or other methods known to those of skill in the art.

Methods are also provided for editing a target nucleic acid molecule, e.g., a single nucleobase within a genome, with an adenine base editor described herein, with reduced off-target effects, e.g., editing of cellular mRNA. In some embodiments, the disclosed DNA editing methods result in an average adenosine (A) to inosine (I) editing frequency in mRNA of 0.3% or less, 0.2% or less, 0.15% or less, or 0.1% or less when contacted with a DNA comprising a target sequence.

In some embodiments, the disclosed editing methods result in an actual or average off-target DNA editing frequency of less than 1.5%, less than 1.25%, less than 1.1%, less than 1%, less than 0.75%, less than 0.5%, less than 0.4%, less than 0.25%, less than 0.2%, less than 0.15%, or less than 0.1%. In some embodiments, the editing method results in less than 20%, 19%, 18%, 16%, 14%, 12%, 10%, 8%, 6%, 4%, 2%, 1%, 0.5%, 0.2%, or 0.1% indel formation in a nucleic acid (e.g., a DNA) comprising a target sequence. For example, the ABE8e (V106W) base editor may exhibit off-target editing frequencies of 0.32% or less while maintaining on-target editing efficiencies of about 80% or more, in target sequences in mammalian cells.

In certain embodiments, the editing methods described herein result in cutting (or nicking) one strand of the double-stranded DNA, for example, the strand that includes the adenine (A) of the target T:A nucleobase pair opposite the strand containing the target thymine (T) that is being excised. This nicking result serves to direct mismatch repair machinery to the non-edited strand, ensuring that the modified nucleotide is not interpreted as a lesion by the cell's machinery. This nick may be created by the use of a nickase napDNAbp domain in the base editor.

In other aspects, the disclosure provides kits for expressing and/or transducing host cells with an expression construct encoding the base editor and gRNA. It further provides kits for administration of expressed adenine base editors and expressed gRNA molecules to a host cell (such as a mammalian cell, e.g., a human cell). The disclosure further provides cells stably or transiently expressing the adenine base editor and gRNA, or a complex thereof. The disclosure further provides cells comprising vectors encoding any of the adenine base editors described herein.

In some embodiments, methods of treatment using the adenine base editors described herein are provided. The methods described herein may comprise treating a subject having or at risk of developing a disease, disorder, or condition associated with a G:C to A:T point mutation comprising administering to the subject an adenine base editor as described herein, a polynucleotide as described herein, a vector as described herein, or a pharmaceutical composition as described herein. In some embodiments, methods of treatment of hemoglobinopathies using the adenine base editors described herein are provided.

The disclosure provides a new phage-assisted continuous evolution (PACE) ABE selection system. Accordingly, in some aspects, the disclosure provides vector systems for performing directed evolution of one or more domains of an base editor (e.g., the adenosine deaminase domain) to engineer any of the disclosed adenine base editors. In some embodiments, the disclosed PACE vector systems comprise a selection plasmid comprising an expression construct encoding a base editor comprising an adenosine deaminase protein and a sequence encoding the N-terminal and C-terminal portions of a split intein (e.g., an Npu split intein), and three accessory plasmids. In some embodiments, the PACE vector systems comprise, in addition to a selection plasmid, one or more accessory plasmids. In some embodiments, the one or more accessory plasmids comprise (1) a first accessory plasmid comprising an isolated nucleic acid comprising an expression construct comprising a sequence encoding a M13 phage gIII protein signal peptide operably controlled by a T7 RNA promoter, wherein the sequence encoding the gIII protein signal peptide lacks one or more nucleic acid bases of the signal peptide domain; (2) a second accessory plasmid encoding the C-terminal portion of a split intein and a sequence encoding a dCas9; and (3) a third accessory plasmid comprising a sequence encoding a T7 RNA polymerase (RNAP) comprising mutations that give rise to two stop codons that can be corrected upon successful base editing.

The details of one or more embodiments of the invention are set forth herein. Other features, objects, and advantages of the invention will be apparent from the Detailed Description, Examples, Figures, and Claims. References cited in this application are incorporated herein by reference in their entireties.

### BRIEF DESCRIPTIONS OF THE DRAWINGS

FIGS. 1A-1D show the phage-assisted evolution of a deoxyadenosine deaminase. FIG. 1A is a schematic of a general PACE overview for base editor evolution^{12, 13}. E. coli host cells contain a plasmid-based genetic circuit that links expression of gene III (gIII, encoding pIII) to the activity of the base editor encoded in a modified M13 bacteriophage. The production of infectious progeny phage requires expression of gene III, which only occurs in host cells infected by phage variants that encode active base editors. Phage exist in a fixed-volume vessel (the lagoon) continuously diluted with host-cell culture, so only those phage that propagate faster than the rate of dilution can persist and evolve. FIG. 1B is a schematic of the selection circuit in PANCE or PACE for evolving the deoxyadenosine deaminase component of ABEs. Plasmid P1 contains gene III driven by a T7 promoter and a sgRNA driven by a Lac promoter. Plasmid P2 expresses catalytically dead Cas9 (dCas9) fused to an N-intein, which forms full-length base editor upon trans-intein splicing with TadA fused to a C-intein (encoded on the SP). Plasmid P3 expresses a T7 RNAP that contains two stop codons that can be corrected to arginine and glutamine upon adenine base editing; this editing event drives expression of gene III. Eight P3 variants (P3a-h) were developed with different promoters and ribosome binding sites (RBS) to tune the stringency of PANCE/PACE. The phage genome is continuously mutated by expression of mutagenic genes from the mutagenesis plasmid (MP). FIG. 1C shows that T7 RNA polymerase (T7 RNAP), which is required for gene III expression from the T7 promoter, contains two stop codons through GC-to-AT mutations at R57 and Q58. Deamination of both adenines by ABE converts the stop codons back to Arg and Gln, resulting in active T7 RNAP and gene III expression. FIG. 1D is a graph showing overnight phage propagation assays to test the activity of different phage pools in host cells harboring P1, P2, and eight different variants of P3 (P3a-h) of increasing stringency. Phage pools were mixed with an excess of log-phase host cells, and the phage were allowed to propagate overnight. To calculate the fold phage propagation, the output phage titer was divided by the input titer. For all plots, dots represent individual biological replicates, bars represent mean values, and error bars represent the standard deviation of three independent biological replicates.

FIGS. 2A-2G show the mutations and kinetics of TadA-8e, as well as the editing characteristics of ABE8e in human cells. FIG. 2A shows the conserved mutations after 25 passages of PANCE (PANCE round 1=passages 1-15; and PANCE round 2=passages 11-25), and genotypes of five TadA variants emerging from 84 hours of PACE (surviving ˜10¹⁷⁴-fold total dilution). For a list of all evolved TadA genotypes, see FIG. 20. FIG. 2B shows E. coli TadA deaminase (left shaded area, PDB 1Z3A) aligned with the structure of S. aureus TadA (not shown) complexed with tRNAA^{rg2} (right shaded area/grey, PDB 2B3J). Mutations evolved during PANCE and PACE are colored to correspond to those in FIG. 20. FIG. 2C shows the architecture of ABE7.10 (ABEmax)¹⁶ and ABE8e. FIG. 2D shows, on the left, representative denaturing polyacrylamide gels of 5′-radiolabeled dsDNA deamination reactions performed with in vitro reconstituted ABE7.10 and ABE8e ribonucleoproteins (RNPs), followed by treatment with EndoV nuclease, which cleaves DNA 3′ of deoxyinosine. FIG. 2D shows, on the right, the fraction of deaminated dsDNA plotted as a function of time in hours or in minutes. The data were fit to a single exponential rate to extract apparent deamination first-order rate constants for ABE7.10 and ABE8e. Data are represented as the mean±s.d. from three independent experiments. FIG. 2E shows base editing in HEK293T cells by SpABE7.10 versus SpABE8e, SaABE7.10 versus SaABE8e, and LbABE7.10 and enAsABE7.10 versus LbABE8e and enAsABE8e base editors, for the two nucleotides with the highest editing efficiency within each protospacer. Bars represent mean values and error bars represent the standard deviation of three independent biological replicates. For editing across the entire protospacer for each site and indel frequencies, see FIGS. 9A-11B. FIG. 2F shows base editing in HEK293T cells by NG-ABE7.10 versus NG-ABE8e and SaKKH-ABE7.10 versus SaKKH-ABE8e, for the two nucleotides with the highest editing within each protospacer. For editing efficiencies across the entire protospacer and indel frequencies, see FIGS. 12A-13B. FIG. 2G shows base editing in HEK293T cells within the protospacer by CP-ABE7.10 and CP-ABE8e variants, compared to SpABE7.10 and SpABE8e. For editing efficiencies across the entire protospacer and indel frequencies, see FIGS. 9A-9B. For all plots, bars represent mean values, and error bars represent the standard deviation of three independent biological replicates.

FIGS. 3A-3E show an off-target analysis of ABE8e. FIG. 3A shows a DNA off-target analysis comparing ABE7.10 plasmid delivery, ABE8e plasmid delivery, and ABE8e RNP delivery at site 5 (HBG), site 6 (VEGFA3), and EMX1. Editing efficiencies and on-target:off-target editing ratios are shown. FIG. 3B shows an off-target transcriptome-wide A-to-I conversion analysis in cellular RNA. FIG. 3C shows DNA editing comparing ABE7.10, ABE8e, and ABE8e(TadA-8e V106W) at seven genomic sites in HEK293T cells. FIG. 3D shows an othogonal R-loop assay overview. FIG. 3E shows Cas9-independent off-target A•T to G•C editing frequencies detected by the orthogonal R-loop assay at each R-loop site with dSaCas9 and a SaCas9 sgRNA. Each R-loop was performed by co-transfection of ABE7.10, ABE8e, or ABE8e (TadA-8e V106W), and a SpCas9 sgRNA targeting site 3 with dSaCas9 and a SaCas9 sgRNA targeting R-loops 1-5, respectively. For all plots, bars represent mean values, and error bars represent the standard deviation of three independent biological replicates.

FIGS. 4A and 4B show adenine base editing with ABE8e at disease-relevant loci in human cells. FIG. 4A shows base editing efficiency and indel frequencies in HEK293T cells at a GATA1 binding site of the BCL11A enhancer by ABE7.10 and ABE8e. Editing efficiencies at each adenine in the protospacer individually and efficiencies of editing both adenines within the same allele. FIG. 4B shows base editing efficiencies and indel frequencies in HEK293T cells of the HBG1/2 promoter with ABE7.10 and ABE8e. Data are shown for each sgRNA provided individually, and for dual (multiplexed) editing of both target sites within a single allele when both sgRNAs are provided simultaneously. Protospacers are named based on the position of the target adenine relative to the HBG transcription start site. PAM sequences are indicated in boxes. For all plots, bars represent mean values and error bars represent the standard deviation of three independent biological replicates.

FIG. 5 shows a PACE schedule for deoxyadenosine deaminase evolution. Lagoon L1 contains host cells harboring P1, P2, and P3e. Lagoons L2 and L3 contain host cells harboring P1, P2, and P3g, which form a more stringent selection circuit than the circuit in lagoon L1. For details on plasmids, see FIG. 18. The stringency of the ABE selection was further modulated by increasing the lagoon flow rate (dashed lines). For the first 12 hours, gene III was expressed by the addition of anhydrotetracycline to enable genetic drift in the absence of selection pressure^{12, 13}.

FIGS. 6A and 6B show base editing efficiencies and indel frequencies for SpABE7.10 and SpABE8a-e variants at four genomic sites in HEK293T cells. FIG. 6A shows base editing with SpABE7.10, SpABE8a, SpABE8b, SpABE8c, SpABE8d, and SpABE8e at four genomic sites in HEK293T cells. PAM sequences are indicated in boxes. FIG. 6B shows the percent of all sequencing reads containing an indel following modification by SpABE7.10, SpABE8a, SpABE8b, SpABE8c, SpABE8d, or SpABE8e at four genomic sites in HEK293T cells. All base editors are optimized with the architecture, codon usage, and nuclear localization signals of ABEmax¹⁶. All untreated controls show no editing or indel formation at all sites. For all plots, dots represent individual biological replicates, and bars represent mean±s.d. of three independent biological replicates.

FIGS. 7A and 7B show base editing efficiencies and indel frequencies for SaABE7.10 and SaABE8a-e variants at four genomic sites in HEK293T cells. FIG. 7A shows base editing with SaABE7.10, SaABE8a, SaABE8b, SaABE8c, SaABE8d, and SaABE8e at four genomic sites in HEK293T cells. PAM sequences for SaCas9 are indicated in boxes. FIG. 7B shows a percent of all sequencing reads containing an indel following modification by SaABE7.10, SaABE8a, SaABE8b, SaABE8c, SaABE8d, or SaABE8e at four genomic sites in HEK293T cells. All base editors are optimized with the architecture, codon usage, and nuclear localization signals of ABEmax¹⁶. All untreated controls show no editing or indel formation at all sites. For all plots, dots represent individual biological replicates and bars represent mean±s.d. of three independent biological replicates.

FIGS. 8A and 8B show base editing efficiencies and indel frequencies for LbABE7.10 and LbABE8a-e variants at three genomic sites in HEK293T cells. FIG. 8A shows base editing with LbABE7.10, LbABE8a, LbABE8b, LbABE8c, LbABE8d, and LbABE8e at three genomic sites in HEK293T cells. PAM sequences for LbCas12a are indicated in boxes. FIG. 8B shows a percent of all sequencing reads containing an indel following modification by LbABE7.10, LbABE8a, LbABE8b, LbABE8c, LbABE8d, or LbABE8e at three genomic sites in HEK293T cells. LbCas12a was used in the nuclease inactive form since the desired nickase enzyme for base editing has not been reported. All base editors are optimized with the architecture, codon usage, and nuclear localization signals of ABEmax¹⁶. All untreated controls show no editing or indel formation at all sites. For all plots, dots represent individual biological replicates, and bars represent mean±s.d. of three independent biological replicates.

FIGS. 9A and 9B show base editing efficiencies and indel frequencies for SpABE7.10, SpABE8e, CP1028-ABE7.10, CP1028-ABE8e, CP1028-ABE8e-dimer, CP1041-ABE7.10, CP1041-ABE8e, and CP1041-ABE8e-dimer at seven genomic sites in HEK293T cells. FIG. 9A shows base editing with SpABE7.10, SpABE8e, CP1028-ABE7.10, CP1028-ABE8e, CP1028-ABE8e-dimer, CP1041-ABE7.10, CP1041-ABE8e, and CP1041-ABE8e-dimer at seven genomic sites in HEK293T cells. FIG. 9B shows a percent of all sequencing reads containing an indel following modification by SpABE7.10, SpABE8e, CP1028-ABE7.10, CP1028-ABE8e, CP1028-ABE8e-dimer, CP1041-ABE7.10, CP1041-ABE8e, or CP1041-ABE8e-dimer at seven genomic sites in HEK293T cells. For all plots, dots represent individual biological replicates, and bars represent mean±s.d. of three independent biological replicates.

FIGS. 10A and 10B show base editing efficiencies and indel frequencies for SaABE7.10, SaABE8e, SaABE8e-dimer, SaKKH-ABE7.10, SaKKH-ABE8e, and SaKKH-ABE8e-dimer in HEK293T cells at five genomic sites with NNGRRT PAMs in HEK293T cells. FIG. 10A shows base editing with SaABE7.10, SaABE8e, SaABE8e-dimer, SaKKH-ABE7.10, SaKKH-ABE8e, and SaKKH-ABE8e-dimer in HEK293T cells at five genomic sites with NNGRRT PAMs in HEK293T cells. PAM sequences for SaCas9 are indicated in boxes. FIG. 10B shows a percent of all sequencing reads containing an indel following modification by SaABE7.10, SaABE8e, SaABE8e-dimer, SaKKH-ABE7.10, SaKKH-ABE8e, and SaKKH-ABE8e-dimer at five genomic sites in HEK293T cells. For all plots, dots represent individual biological replicates, and bars represent mean±s.d. of three independent biological replicates.

FIGS. 11A and 11B show base editing efficiencies and indel frequencies for LbABE7.10, LbABE8e, Lb-ABE8e-dimer, enAsABE7.10, enAsABE8e, and enAsABE8e-dimer in HEK293T cells at three genomic sites in HEK293T cells. FIG. 11A shows base editing with LbABE7.10, LbABE8e, LbABE8e-dimer, enAsABE7.10, enAsABE8e, and enAsABE8e-dimer in HEK293T cells at three genomic sites in HEK293T cells. PAM sequences for LbCas12a are indicated in boxes. FIG. 11B shows a percent of all sequencing reads containing an indel following modification by LbABE7.10, LbABE8e, LbABE8e-dimer, enAsABE7.10, enAsABE8e, and enAsABE8e-dimer at three genomic sites in HEK293T cells. For all plots, dots represent individual biological replicates, and bars represent mean±s.d. of three independent biological replicates.

FIGS. 12A and 12B show base editing efficiencies and indel frequencies for NG-ABE7.10, NG-ABE8e, and NG-ABE8e-dimer in HEK293T cells at five genomic sites in HEK293T cells. FIG. 12A shows base editing with NG-ABE7.10, NG-ABE8e, and NG-ABE8e-dimer in HEK293T cells at five genomic sites in HEK293T cells. FIG. 12B shows a percent of all sequencing reads containing an indel following modification by NG-ABE7.10, NG-ABE8e, and NG-ABE8e-dimer at five genomic sites in HEK293T cells. For all plots, dots represent individual biological replicates and bars represent mean±s.d. of three independent biological replicates.

FIGS. 13A and 13B show base editing efficiencies and indel frequencies for SaABE7.10, SaABE8e, SaABE8e-dimer, SaKKH-ABE7.10, SaKKH-ABE8e, and SaKKH-ABE8e-dimer in HEK293T cells at nine genomic sites with NNHRRT PAMs in HEK293T cells. FIG. 13A shows base editing with SaABE7.10, SaABE8e, SaABE8e-dimer, SaKKH-ABE7.10, SaKKH-ABE8e, and SaKKH-ABE8e-dimer in HEK293T cells at nine genomic sites with NNHRRT PAMs in HEK293T cells. PAM sequences for SaCas9 are indicated in boxes. FIG. 13B shows a percent of all sequencing reads containing an indel following modification by SaABE7.10, SaABE8e, SaABE8e-dimer, SaKKH-ABE7.10, SaKKH-ABE8e, and SaKKH-ABE8e-dimer at nine genomic sites in HEK293T cells. For all plots, dots represent individual biological replicates and bars represent mean±s.d. of three independent biological replicates.

FIG. 14 shows a processivity analysis for ABE7.10 and ABE8e. Percent of all sequenced alleles in which the two target As in the editing window are both converted to Gs, following transfection with plasmids expressing ABE7.10 or ABE8e at three genomic sites in HEK293T cells. Analyses were done using the Python script provided in Example 4, Supplementary Note 1, which is outlined below. Dots represent individual values and bars represent mean±s.d. of three independent biological replicates.

FIG. 15 shows a comparison of ABE8e and Cas9 nuclease at the top three gRNA-dependent off-target sites for EMX1 as identified by GUIDE-Seq⁴⁴.

FIG. 16 shows allele compositions following treatment with ABE7.10 or ABE8e at a GATA1 binding site of the BCL11A enhancer. Due to increased processivity and overall efficiency, an increased percentage of alleles simultaneously containing A•T-to-G•C editing at positions four and seven was observed after treatment with ABE8e compared to ABE7.10. Multiple edits were predicted to more effectively disrupt the GATA1 binding site compared to single edits⁴⁹. The data shown is representative of three biological replicates.

FIG. 17 shows allele compositions following treatment with ABE7.10 or ABE8e at the HBG1 and HBG2 promoters. The data shown is representative of three biological replicates. The target sites at −175T and −198T in the promoters are indicated.

FIG. 18 is a table showing the plasmid-based genetic circuit components for the PACE experiments.

FIG. 19 is a table showing the selection schedule for PANCE. For the first phase of PANCE (PANCE 1), two selections were performed in parallel using host cells harboring P1, P2, and either P3a or P3b. For the second phase of PANCE (PANCE 2), four selections were performed in parallel, two in host cells harboring P1, P2, and P3e, and two in host cells harboring P1, P2, and P3g. For details on plasmids, see FIG. 18. Following the final passage of PANCE 2 (25 total passages), all phage were pooled and used as the starting point for PACE.

FIG. 20 shows a mutation table of variants from PANCE and PACE. Data were obtained by sequencing individual plaques. Conserved mutations are bolded. Mutations that are highlighted in the structure in FIG. 2B are highlighted to match the amino acid positions in the structure. PACE, Pool 2, Variants 1, 4, 6, and 7, and PACE, Pool 3, Variant 6 were tested for base editing activity in mammalian cells.

FIG. 21 is a table showing protospacer sequences for mammalian genomic sites targeted in Examples 1-4.

FIGS. 22A and 22B show a table of primers used in the mammalian cell genomic DNA amplification of Examples 1-4. The table is continued across FIGS. 22A-22B.

FIGS. 23A-23F show a table of amplicons used in the high-throughput sequencing analyses of Examples 1-4. The table is continued across FIGS. 23A-23F.

FIG. 24 shows titration data at eight editor doses comparing base editing efficiencies for ABE8e and ABE8e-dimer at three sites in HEK293T cells. Base editing with ABE8e and ABE8e-dimer in HEK293T cells at three genomic sites in HEK293T cells. Transfections were performed with a constant amount of sgRNA plasmid but eight varying doses of ABE plasmid. For all plots, dots represent individual biological replicates, and bars represent mean±s.d. of three independent biological replicates. Editing patterns are indicated on the plot with symbols (>, *).

FIG. 25 shows on-target DNA base editing efficiencies comparing ABE8e mutants with minimized transcriptome-wide RNA off-target editing. Base editing with Cas9(D10A), ABE7.10, ABE8e, ABE8e(TadA-8e V106W), ABE8e(TadA-8e V82G), and ABE8e(TadA-8e K20A R21A) at the LDLR locus in HEK293T cells⁴⁷. Editing was analyzed from RNAseq analysis. All base editors are optimized with the architecture, codon usage, and nuclear localization signals of ABEmax¹⁶. For all plots, dots represent individual biological replicates, and bars represent mean±s.d. of three independent biological replicates. The PAM sequence is indicated in a box.

FIGS. 26A and 26B show on-target base editing efficiencies and indel frequencies for ABE7.10, ABE8e, and ABE8e(TadA-8e V106W) in HEK293T cells at seven genomic sites in HEK293T cells. FIG. 26A shows base editing with ABE7.10, ABE8e, and ABE8e(TadA-8e V106W) in HEK293T cells at seven genomic sites in HEK293T cells. FIG. 26B shows the percent of all sequencing reads containing an indel following modification by ABE7.10, ABE8e, and ABE8e(TadA-8e V106W) at seven genomic sites in HEK293T cells. All untreated controls show no editing or indel formation at all sites. For all plots, dots represent individual biological replicates, and bars represent mean±s.d. of three independent biological replicates.

FIG. 27 shows on-target base editing efficiencies and indel frequencies for ABE7.10, ABE8e, and ABE8e(TadA-8e V106W) in HEK293T cells at site 3 in HEK293T cells for the orthogonal R-loop assay. Base editing with ABE7.10, ABE8e, and ABE8e (TadA-8e V106W) in HEK293T cells at site 3. For all plots, dots represent individual biological replicates, and bars represent mean±s.d. of three independent biological replicates. The PAM sequence is boxed.

FIG. 28 depicts an alignment of the amino acid sequences of TadA deaminases derived from various species and TadA-8e (derived from E. coli) with the consensus E. coli TadA sequence.

FIGS. 29A and 29B show a TadA-8e V106W target editing analysis for SaCas9 and LbCas12a. FIG. 29A is a graph of DNA editing comparing SaABE7.10, SaABE8e, and SaABE8e (TadA-8e V106W) at seven genomic sites in HEK293T cells. FIG. 29B is a graph of DNA editing comparing LbABE7.10, LbABE8e, and LbABE8e (TadA-8e V106W) at seven genomic sites in HEK293T cells.

FIGS. 30A-30H show that adenine base editing converts the sickle cell disease β-globin allele (HBB^{S}) to the benign Makassar variant (HBB^{G}) in patient CD34+ hematopoietic stem and progenitor cells (HSPCs). CD34+ cells from three SCD patient donors were electroporated with ABE8e-NRCH mRNA or RNP using a single guide RNA (sgRNA) targeting the SCD mutant codon. FIG. 30A: The amino acid and nucleotide sequence of the edited region, with the target adenosine (A7) and potential bystander edits (A9, A12, A16) indicated with underline, with amino acid changes indicated. Bystander editing at A9 and A12 results in synonymous mutations. The protospacer and the 5′-CACC-3′ PAM are underlined. Nucleotides are numbered starting at the 5′ end of the protospacer. FIG. 30B: Editing efficiencies at all target and bystander nucleotides, and indels, as measured by high-throughput DNA sequencing (HTS) after 6 days of in stem cell culture media following electroporation. FIG. 30C: Proportion of β-like globin proteins quantified by reverse-phase high performance liquid chromatography (RP-HPLC) of reticulocyte lysates after 18 days of culture in differentiation media following electroporation. FIG. 30D: Representative phase contrast images of reticulocytes incubated for 8 hours in 2% 02. An unedited reticulocyte population derived from healthy donor (HBBA/A) CD34+ HSPCs was included for comparison alongside those derived from unedited, mRNA-edited and RNP-edited SCD HBB^{S/S} CD34+ HSPCs. Scale bar=50 μm. FIG. 30E: Quantification of sickled reticulocytes calculated by counting images after incubation for 8 hours in 2% 02 such as in FIG. 30D. More than 300 randomly selected cells per sample were counted by a blinded observer. FIG. 30F: Venn diagram showing candidate off-target sites nominated by the algorithms Cas-OFFinder and CIRCLE-seq, and nominated sites for which off-target editing was observed by targeted DNA sequencing in CD34+ cells from three SCD patient donors electroporated with ABE8e-NRCH mRNA. FIG. 30G: Pie chart showing the fraction of validated off-target sites according to their predicted genomic annotation using HOMER. TSS, transcription start site, defined as nucleotides 1 kb upstream to 100 bp downstream of transcription start site; TTS, transcription termination site, defined as nucleotides 100 bp upstream to 1 kb downstream of the transcription termination site. FIG. 30H: Histogram showing the number of validated base editing off-target sites according to the frequency of reads with one or more A•T to G•C conversions. Bar graphs in FIGS. 30B, 30C, and 30E show mean values±standard deviation of three independent biological replicates, with individual values shown as dots.

FIGS. 31A-31H show that engraftment of ABE8e-NRCH mRNA-treated SCD patient CD34+ HSPCs after transplantation into immunodeficient mice. CD34+ HSPCs from three HBB^{S/S} SCD patient donors were electroporated with ABE8e-NRCH mRNA and a single guide RNA (sgRNA) targeting the SCD mutant codon, followed by transplantation of 2-5×10⁵ treated cells into NBSGW mice via tail-vein injection. Mice were sacrificed and analyzed 16 weeks after transplantation. FIG. 31A: Experimental workflow. FIG. 31B: Engraftment measured by the percentage of human donor CD45+ cells (hCD45+ cells) in recipient mouse bone marrow. FIG. 31C: Human B-cells (hCD19+), myeloid cells (hCD33+), and T-cells (hCD3+) cells in recipient mouse bone marrow, shown as percentages of the total hCD45+ population. FIG. 31D: Human erythroid precursors (hCD235a+) in recipient mouse bone marrow shown as percentage of total human and mouse CD45− cells. FIG. 31E: On-target editing efficiencies in human donor CD34+ cell-derived lineages purified from recipient bone marrow by fluorescence-activated cell sorting. Erythroid, myeloid, B-cell, and HSPC human lineages were collected using antibodies that recognize hCD235a, hCD33, hCD19, and hCD34+, respectively. Statistical significance was assessed by one-way ANOVA to compare groups; “ns”, not significant. Cell types are indicated in the legend with symbols (>, *, \hat{ }). FIG. 31F: Percentages of β-like globin proteins determined by reverse-phase HPLC analysis of human donor-derived reticulocytes isolated from recipient mouse bone marrow. FIG. 31G: Representative phase contrast images of human reticulocytes purified from bone marrow and incubated for 8 hours in 2% 02. Scale bar=25 μm. FIG. 31H: Quantification of sickled cells calculated by counting images after incubation for 8 hours in 2% 02 as in FIG. 31G. More than 300 randomly selected cells per sample were counted by a blinded observer. n=14 total mice analyzed in FIGS. 31B-31F; triangle, square, and circle symbols represent samples from three different SCD CD34+ HSPC donors. Graphs show mean values±standard deviation. Statistical significance between treated and untreated samples was assessed by a two-tailed Student's t-test; “ns”, not significant.

FIGS. 32A-32H show that adenine base editing of the sickle cell disease β-globin allele (HBB^{S}) to the benign Makassar variant (HBB^{G}) alleviates hematologic pathology in a mouse model of SCD. FIG. 32A: Diagram detailing the experimental workflow. Lineage negative (Lin⁻) HSPCs were isolated from the bone marrow of Townes SCD mice (CD45.2, HBB^{S}/HBB^{S}), electroporated with ABE8e-NRCH and sgRNA ribonucleoprotein complex (RNP) or mock electroporated with no base editor as a control, then transplanted into irradiated congenic CD45.1 C57B1/6 recipient mice. Peripheral blood was analyzed serially at 6, 10, 14, and 16 weeks after transplantation to determine the fraction of engrafted donor cells and hemoglobin content. Mice were sacrificed for necropsy at 16 weeks. As a control for healthy RBCs, unedited HBB^{S/A} (sickle cell trait) HSPCs were transplanted into irradiated congenic CD45.1 C57B1/6 recipient mice. FIG. 32B: Donor cell engraftment measured by flow cytometry assessing CD45.1 expression on peripheral blood mononuclear cells (PBMCs). Editing patterns are indicated on the plot with symbols (>, *, \hat{ }) FIG. 32C: HBB^{S}-to-HBB^{G} editing efficiency measured by HTS of genomic DNA in cells cultured 3 days after electroporation (pre-transplantation) or in PBMCs collected at 16 weeks (after transplantation). Statistical significance was assessed by a two-tailed Student's t-test, “ns” not significant. FIG. 32D: The percentages of PG globin protein in blood measured by reverse-phase HPLC at each blood draw. Editing patterns are indicated on the plot with symbols (>, *, \hat{ }). FIGS. 32E-32H: Hematologic properties measured using a veterinary blood analyzer instrument. Blood from non-transplanted HBB^{A/S} Townes mice were also measured as a healthy, non-transplanted animal control. Statistical significance between groups of mice were assessed using one-way ANOVA, with Sidák's multiple comparisons test of the edited HBB^{S/S} values compared to each other group to calculate p-values. No statistically significant differences were observed between edited HBBS/S and either transplanted or non-transplanted HBB^{A/S} mice. FIG. 32E: Blood hemoglobin levels. FIG. 32F: White blood cell count. FIG. 32G: Red blood cell count. FIG. 32H: Reticulocytes as a percentage of total cells in blood. Error bars indicate standard deviation. Unedited HBB^{S/S}: n=6 mice. Edited HBB^{S/S}: n=6 mice. HBB^{A/S}: n=2 mice. HBB^{A/S} not transplanted: n=5 mice.

FIGS. 33A-33F show that adenine base editing of the sickle cell disease β-globin allele (HBB^{S}) to the Makassar variant (HBB^{G}) reduces erythrocyte sickling and splenic pathologies in mice. Mice were treated as described in FIG. 32A. Blood and spleen were analyzed sixteen weeks after transplantation of edited Lin− HSPCs. FIG. 33A: Representative images of blood smears. Scale bar=25 μm FIG. 33B: Representative phase contrast images of peripheral blood incubated for 8 hours in 2% 02. Scale bar=50 μm FIG. 33C: Quantification of sickled cells. More than 300 randomly selected cells were counted by a blinded observer. FIG. 33D: Mass of dissected spleens. FIG. 33E: Representative images of spleens isolated from mice transplanted with Lin− HSPCs of the indicated genotype, with a ruler at top for scale. FIG. 33F: Histological sections of spleens of recipient mice 16 weeks after transplantation. Splenic pathologies in mice that received unedited donor HBB^{S/S} HSCs include excessive extramedullary erythropoiesis evidenced by increased red pulp (RP) and vascular congestion indicated by RBC pooling (arrows). White pulp (WP), indicated by dotted lines, appears darker staining than red pulp. Images were taken at 10× magnification and were processed, stained and photographed at the same time under identical conditions. Scale bar=100 μm. Unedited HBB^{S/S}: n=6 mice; edited HBB^{S/S}: n=6 mice; HBB^{A/S}: n=2 mice. Graphs show mean values±standard deviation, with individual values shown as dots. Statistical significance was assessed using one-way ANOVA, with Sidák's multiple comparisons test of the edited HBB^{S/S} values compared to each other group to calculate p-values.

FIG. 34 shows optimization of HBB^{S}-to-HBB^{G} base editing in HEK293T cells. Plasmids encoding the HBB^{S}-targeting sgRNA and either ABE7.10-NRCH or ABE8e-NRCH were transfected by lipofection into HEK293T cells. Editing efficiency was measured after 3 days by high-throughput DNA sequencing (HTS).

FIG. 35 shows cell viability and recovery of CD34+ HSPCs after electroporation. Two days after electroporation of base editor mRNA and sgRNA, or electroporation of ribonucleoprotein (RNP), cell number and viability were measured using a Chemometec Nucleocounter-3000. Acridine orange was used to stain the total cell number and DAPI was used to stain dead, permeabilized cells. The percent viability was calculated as the DAPI stained cells divided by the acridine orange cells within each sample. The percent recovery was normalized to the cell count of the unedited sample. Unedited cells were not electroporated. Error bars show standard deviation, n=3.

FIG. 36 shows erythroid differentiation of edited CD34+ HSPCs. Representative, immuno-flow cytometry for erythroid maturation stage markers at culture days 7 and 14. Top: gating strategy to identify single cells expressing the erythroid marker hCD235a. Bottom: gating strategy to track the progress of erythroid maturation based on expression of CD49D and Band3 in hCD235a+ cells. SSC-A: Side scatter area. SSC-W: Side scatter width. FSC-A: Forward scatter area.

FIG. 37 shows the results of reverse-phase high performance liquid chromatography (HPLC) analysis of erythroid cells derived from edited CD34+ HSPCs. Representative reverse-phase HPLC chromatograms of erythroid cell lysates at culture day 18, with β-like globins and their associated fractions marked near the associated peak.

FIGS. 38A and 38B show the extent of off-target base editing associated with ABE8e-NRCH conversion of HBB^{S} to HBB^{G} Makassar in sickle cell disease patient CD34+ hematopoietic stem and progenitor cells. FIG. 38A: CIRCLE-seq read counts obtained for each verified off-target site and the alignment of each site to the guide sequence. FIG. 38B: Bar graph showing the percentage of sequencing reads containing A:G to G:C edited alleles at on-and off-target sites in genomic DNA samples from patient CD34+ HSPCs treated with ABE8e-NRCH mRNA, ribonucleoprotein (RNP), or untreated controls (n=4). Asterisks (*) indicate sites that were identified by both Cas-OFFinder and CIRCLE-seq methods. Each triplet of bars represents, from top to bottom, mRNA delivery, RNP delivery, and control.

FIG. 39 show the extent of off-target indel formation associated with ABE8e-NRCH-mediated conversion of HBB^{S} to HBB^{G} Makassar in sickle cell disease patient CD34+ hematopoietic stem and progenitor cells. Bar graphs show the percentage of sequencing reads containing alleles harboring indels at on-and off-target sites in genomic DNA samples from patient CD34+ HSPCs treated with ABE8e-NRCH mRNA, ribonucleoprotein (RNP), or untreated controls (n=4). Asterisks (*) indicate sites that were identified by both Cas-OFFinder and CIRCLE-seq methods. Each triplet of bars represents, from top to bottom, mRNA delivery, RNP delivery, and control.

FIGS. 40A-40H show engraftment of ABE8e-NRCH RNP-treated SCD patient CD34+ HSPCs after transplantation into immunodeficient mice. CD34+ HSPCs from three HBB^{S/S} SCD patient donors were electroporated with ABE8e-NRCH RNP using a single guide RNA (sgRNA) targeting the SCD mutant codon, followed by transplantation of 2-5×10⁵ treated cells into NBSGW mice via tail-vein injection. Mice were sacrificed and analyzed 16 weeks after transplantation. FIG. 40A: Experimental workflow. FIG. 40B: Engraftment measured by the percentage of human donor CD45+ cells (hCD45+ cells) in recipient mouse bone marrow. FIG. 40C: Human B-cells (hCD19+), myeloid cells (hCD33+), and T-cells (hCD3+) cells in recipient mouse bone marrow, shown as percentages of the total hCD45+ population. FIG. 40D: Human erythroid precursors (hCD235a+) in recipient mouse bone marrow shown as percentage of total human and mouse CD45− cells. FIG. 40E: On-target (A7, FIG. 30A) editing efficiencies in human donor CD34+ cell-derived lineages purified from recipient bone marrow by fluorescence-activated cell sorting. Erythroid, myeloid, B-cell, and HSPC human lineages were collected using antibodies that recognize hCD235a, hCD33, hCD19, and hCD34+, respectively. Statistical significance was assessed by one-way ANOVA to compare groups; “ns”, not significant. Cell types are indicated in the legend with symbols (>, *, \hat{ }). FIG. 40F: Percentages of β-like globin proteins determined by reverse-phase HPLC analysis of human donor-derived reticulocytes isolated from recipient mouse bone marrow. FIG. 40G: Representative phase contrast images of human reticulocytes purified from bone marrow and incubated for 8 hours in 2% 02. Scale bar=25 μm. FIG. 40H: Quantification of sickled cells calculated by counting images after incubation for 8 hours in 2% 02 such as in FIG. 40G. More than 300 randomly selected cells per sample were counted by a blinded observer. n=14 total mice analyzed in FIGS. 40B-40F; triangle, square, and circle symbols represent samples from three different SCD CD34+ HSPC donors. Graphs show mean values±standard deviation. Statistical significance between treated and untreated samples was assessed by a two-tailed Student's t-test; “ns”, not significant.

FIG. 41 shows a flow cytometry analysis of human CD34+ donor-derived hematopoietic cells after transplantation into NBSGW mice. Flow cytometry gating strategies for transplantation studies. Recipient mice were sacrificed at 16 weeks. Human donor cell-derived lineages in recipient bone marrow were identified by immune-flow cytometry using the indicated human (h) hematopoietic cell-specific antibodies.

FIG. 42 shows a flow cytometry analysis of human donor-derived erythroid CD235a+ cells after transplantation. Human CD235a+ erythroid cells were purified by immuno-magnetic bead selection and analyzed by flow cytometry for the indicated erythroid maturation markers (top panel). Enucleated reticulocytes were assessed by the cell-permeable DNA stain Hoechst 33342 and the erythroid marker CD235a (bottom panel).

FIG. 43 shows a plot of the oxygen binding affinity of blood from sickle cell disease model mice following transplantation of edited cells. Hemoglobin oxygenation was measured using a Hemox Analyzer (TCS Scientific) across a continuous declining gradient of oxygen pressure to assess whether HBB^{S}-to-HBB^{G} editing led to altered hemoglobin-oxygen binding. Blood was drawn from mice at week 14 after transplantation. Editing patterns are indicated on the plot with symbols (>, *, \hat{ })

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

“Base editing” refers to genome editing technology that involves the conversion of a specific nucleic acid base into another at a targeted genomic locus. In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g. typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein.

The following base editor, which effects transitions (pyrimidine to pyrimidine, or purine to purine) mutations are relevant to the methods disclosed herein. Adenine base editor (or “ABE”). This type of editor converts an A:T Watson-Crick nucleobase pair to a G:C Watson-Crick nucleobase pair. Because the corresponding Watson-Crick paired bases are also interchanged as a result of the conversion, this category of base editor may also be referred to as a thymine base editor (or “TBE”).

The term “base editor (BE)” as used herein, refers to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid such as a base within a DNA molecule. In the case of an adenine base editor, the base editor is capable of deaminating an adenine (A) in DNA. Such base editors may include a nucleic acid programmable DNA binding protein (napDNAbp) fused to an adenosine deaminase. Some base editors include CRISPR-mediated fusion proteins that are utilized in the base editing methods described herein. In some embodiments, the base editor comprises a nuclease-inactive Cas9 (dCas9) fused to a deaminase which binds a nucleic acid in a guide RNA-programmed manner via the formation of an R-loop, but does not cleave the nucleic acid. For example, the dCas9 domain of the base editor may include a D10A and a H840A mutation (which renders Cas9 capable of cleaving only one strand of a nucleic acid duplex), as described in PCT/US2016/058344, which published as WO 2017/070632 on Apr. 27, 2017 and is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand”, or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821(2012); Qi et al., Cell. 28; 152(5):1173-83 (2013)).

The term “base editor” encompasses the CRISPR-mediated fusion proteins utilized in the multiplexed base editing methods described herein as well as any base editor known or described in the art at the time of this filing or developed in the future. Reference is made to Rees & Liu, Base editing: precision chemistry on the genome and transcriptome of living cells, Nat. Rev. Genet. 2018; 19(12):770-788; as well as U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; International Publication No. WO 2018/027078, published Aug. 2, 2018; International Application No PCT/US2018/056146, filed Oct. 16, 2018, which published as Publication No. WO 2019/079347 on Apr. 25, 2019; International Application No PCT/US2019/033848, filed May 23, 2019, which published as Publication No. WO 2019/226953 on Nov. 28, 2019; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2019/023680, published Jan. 31, 2019; International Publication No. WO 2018/0176009, published Sep. 27, 2018; International Publication No. WO 2020/041751, published Feb. 27, 2020; International Publication No. WO 2020/051360, published Mar. 12, 2020; International Patent Publication No. WO 2020/102659, published May 22, 2020; International Publication No. WO 2020/086908, published Apr. 30, 2020; International Publication No. WO 2020/181180, published Sep. 10, 2020; International Publication No. WO 2020/214842, published Oct. 22, 2020; International Publication No. WO 2020/092453, published May 7, 2020; International Patent Application No. PCT/US2020/033873, filed May 20, 2020, published as International Publication No. WO2020/236982 on Nov. 26, 2020, and International Application No. PCT/US2020/062428, filed Nov. 25, 2020, each of which is incorporated herein by reference in its entirety.

The term “Cas12” or “Cas12 nuclease” or “Cas12 domain” refers to a CRISPR-associated protein 12, or variant thereof, and embraces any naturally occurring Cas12 from any organism, any naturally-occurring Cas12 equivalent or fragment thereof, any Cas12 homolog, ortholog, or paralog from any organism, and any variant of a Cas12, naturally-occurring or engineered. Cas12a used to be referred to as Cpf1, thus this term embraces any naturally occurring Cpf1 from any organism, any naturally-occurring Cpf1 equivalent or fragment thereof, any Cpf1 homolog, ortholog, or paralog from any organism, and any variant of a Cpf1, naturally-occurring or engineered. The term Cas12 is not meant to be particularly limiting and may be referred to as a “Cas12 or variant thereof.” Exemplary Cas12 proteins are described herein and also described in the art.

The term “Cas9” or “Cas9 nuclease” or “Cas9 domain” refers to a CRISPR-associated protein 9, or variant thereof, and embraces any naturally occurring Cas9 from any organism, any naturally-occurring Cas9 equivalent or fragment thereof, any Cas9 homolog, ortholog, or paralog from any organism, and any variant of a Cas9, naturally-occurring or engineered. The term Cas9 is not meant to be particularly limiting and may be referred to as a “Cas9 or variant thereof.” Exemplary Cas9 proteins are described herein and also described in the art. The present disclosure is unlimited with regard to the particular Cas9 that is employed in the CRISPR-mediated fusion proteins utilized in the disclosure.

As used herein, the term “dCas9” refers to a nuclease-inactive Cas9 or nuclease-dead Cas9, or a variant thereof, and embraces any naturally occurring dCas9 from any organism, any naturally-occurring dCas9 equivalent or functional fragment thereof, any dCas9 homolog, ortholog, or paralog from any organism, and any variant of a dCas9, naturally-occurring or engineered. The term dCas9 is not meant to be particularly limiting and may be referred to as a “dCas9 or variant thereof.” Exemplary dCas9 proteins and methods for making dCas9 proteins are further described herein and/or are described in the art and are incorporated herein by reference. Any suitable mutation which inactivates both Cas9 endonucleases, such as D10A and H840A mutations in the wild-type S. pyogenes Cas9 amino acid sequence, or D10A and N580A mutations in the wild-type S. aureus Cas9 amino acid sequence, may be used to form the dCas9.

As used herein, the term “nCas9” or “Cas9 nickase” refers to a Cas9 or a variant thereof, which cleaves or nicks only one of the strands of a target cut site thereby introducing a nick in a double strand DNA molecule rather than creating a double strand break. This can be achieved by introducing appropriate mutations in a wild-type Cas9 which inactivates one of the two endonuclease activities of the Cas9. Any suitable mutation which inactivates one Cas9 endonuclease activity but leaves the other intact is contemplated, such as one of D10A or H840A mutations in the wild-type S. pyogenes Cas9 amino acid sequence, or a D10A mutation in the wild-type S. aureus Cas9 amino acid sequence, may be used to form the nCas9.

The term “deaminase” or “deaminase domain” refers to a protein or enzyme that catalyzes a deamination reaction. In some embodiments, the deaminase is an adenosine deaminase, which catalyzes the hydrolytic deamination of the nucleobase adenine. In some embodiments, the adenosine deaminase catalyzes the hydrolytic deamination of adenine in deoxyribonucleic acid (DNA) to hypoxanthine.

The deaminases provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

As used herein, the term “adenosine deaminase domain” refers to a domain within a base editor comprising one or more adenosine deaminase enzymes. For instance, an adenosine deaminase domain may comprise a single adenosine deaminase. In other embodiments, an adenosine deaminase domain may comprise a heterodimer of a first adenosine deaminase and a second deaminase, e.g., wherein the two deaminases are connected by a linker. Adenosine deaminases (e.g., engineered adenosine deaminases or evolved adenosine deaminases) provided herein may be may comprise enzymes that convert adenosine (A) to inosine (I) in DNA. Such adenosine deaminases may cause an A:T to G:C base pair conversion. In some embodiments, the deaminase is a variant of a naturally-occurring deaminase from an organism. In some embodiments, the deaminase does not occur in nature. For example, in some embodiments, the deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

In some embodiments, the adenosine deaminase is derived from a bacterium, such as E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018-0073012, published Mar. 15, 2018, and International Publication No. WO 2019/079347, each of which is incorporated herein by reference.

As used herein, the term “DNA binding protein” or “DNA binding protein domain” refers to any protein that localizes to and binds a specific target DNA nucleotide sequence (e.g., a gene locus of a genome). This term embraces RNA-programmable proteins, which associate (e.g., form a complex) with one or more nucleic acid molecules (i.e., which includes, for example, guide RNA in the case of Cas systems) that direct or otherwise program the protein to localize to a specific target nucleotide sequence (e.g., DNA sequence) that is complementary to the one or more nucleic acid molecules (or a portion or region thereof) associated with the protein. Exemplary RNA-programmable proteins are CRISPR-Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353(6299), the contents of which are incorporated herein by reference.

The term “DNA editing efficiency,” as used herein, refers to the number or proportion of intended base pairs that are edited. For example, if a base editor edits 10% of the base pairs that it is intended to target (e.g., within a cell or within a population of cells), then the base editor can be described as being 10% efficient. Some aspects of editing efficiency embrace the modification (e.g. deamination) of a specific nucleotide within DNA, without generating a large number or percentage of insertions or deletions (i.e., indels). It is generally accepted that editing while generating less than 5% indels (as measured over total target nucleotide substrates) is high editing efficiency. The generation of more than 20% indels is generally accepted as poor or low editing efficiency. Indel formation may be measured by techniques known in the art, including high-throughput screening of sequencing reads.

The terms “RNA editing activity,” “RNA editing effects” and “RNA off-target editing,” as used herein, refer to the introduction of modifications (e.g., deaminations) to nucleotides within cellular RNA, e.g. messenger RNA (mRNA). An important goal of DNA base editing efficiency is the modification (e.g. deamination) of a specific nucleotide within DNA, without introducing modifications of similar nucleotides within RNA. RNA editing effects are “low” or “reduced” when a detected mutation is introduced into RNA molecules at a frequency of 0.3% or less. For reference, the ABEmax base editor introduces edits into RNA at a frequency of about 0.50%. RNA editing effects are “low” or “reduced” when a mutation is detected at a magnitude that is less than about 70,000 edits within an analyzed mRNA transcriptome. The number of RNA edits may be measured by techniques known in the art, including high-throughput screening of sequencing reads and RNA-seq. The effects of RNA editing on the function of a protein translated from the edited mRNA transcript may be predicted by use of the SIFT (“Sorting Intolerant from Tolerant”) algorithm, which bases predictions on sequence homology and the physical properties of amino acids.

The term “on-target editing,” as used herein, refers to the introduction of intended modifications (e.g., deaminations) to nucleotides (e.g., adenine) in a target sequence, such as using the base editors described herein. The term “off-target DNA editing,” as used herein, refers to the introduction of unintended modifications (e.g. deaminations) to nucleotides (e.g. adenine) in a sequence outside the canonical base editor binding window (i.e., from one protospacer position to another, typically 2 to 8 nucleotides long). Off-target DNA editing can result from weak or non-specific binding of the gRNA sequence to the target sequence. As used herein, the term “bystander editing” refers to synonymous off-target point mutations at nucleobases that are near (proximate to) the target base and do not change the outcome of the intended editing method.

As used herein, the term “purity” of a base editor refers to the editor's productive editing frequency, which is calculated as the mean rate at which the contacting of the target sequence with a base editor results in a product that does not contain an amino acid mutation.

The term “Cas9-dependent off-target editing” refers to the introduction of unintended modifications that result from weak or non-specific binding of a napDNAbp-gRNA complex (e.g., a complex between a gRNA and the base editor's napDNAbp domain) to nucleic acid sites that have fairly high (e.g. more than 60%, or having fewer than 6 mismatches relative to) sequence identity to a target sequence. In contrast, the term “Cas9-independent off-target editing” refers to the introduction of unintended modifications that result from weak associations of a base editor (e.g., the nucleotide modification domain) to nucleic acid sites that do not have high sequence identity (about 60% or less, or having 6-8 or more mismatches relative to) to a target sequence. Because these associations occur independent of any hybridization between the Cas9-gRNA complex and the relevant nucleic acid site, they are referred to as “Cas9-independent.”

The term “off-target editing frequency,” as used herein, refers to the number or proportion of unintended base pairs that are edited. On-target and off-target editing frequencies may be measured by the methods and assays described herein, further in view of techniques known in the art, including high-throughput sequencing reads. As used herein, high-throughput sequencing involves the hybridization of nucleic acid primers (e.g., DNA primers) with complementarity to nucleic acid (e.g., DNA) regions just upstream or downstream of the target sequence or off-target sequence of interest. Because the DNA target sequence and the Cas9-independent off-target sequences are known a priori in the methods disclosed herein, nucleic acid primers with sufficient complementarity to regions upstream or downstream of the target sequence and Cas9-independent off-target sequences of interest may be designed using techniques known in the art, such as the PhusionU PCR kit (Life Technologies), Phusion HS II kit (Life Technologies), and Illumina MiSeq kit. The identity and number of off-target DNA edits may be measured by techniques known in the art, including high-throughput screening of sequencing reads, EndoV-Seq, GUIDE-Seq, CIRCLE-Seq, and Cas-OFFinder. Since many of the Cas9-dependent off-target sites have high sequence identity to the target site of interest, nucleic acid primers with sufficient complementarity to regions upstream or downstream of the Cas9-dependent off-target site may likewise be designed using techniques and kits known in the art. These kits make use of polymerase chain reaction (PCR) amplification, which produces amplicons as intermediate products. The target and off-target sequences may comprise genomic loci that further comprise protospacers and PAMs. Accordingly, the term “amplicons,” as used herein, may refer to nucleic acid molecules that constitute the aggregates of genomic loci, protospacers and PAMs. High-throughput sequencing techniques used herein may further include Sanger sequencing and/or whole genome sequencing (WGS).

As used herein, a “ratio of on-target:off-target editing” means a ratio of sequencing reads reflecting on-target deaminations relative to deaminations of known or predicted off-target sites, or candidate off-target sites. Candidate off-target sites may be identified, and hence the ratio of on-target:off-target editing may be measured, using an experimental assay or a computation algorithm (e.g., Cas-OFFinder). For example, candidate off-target sites may be identified using an experimental assay such as EndoV-Seq, GUIDE-Seq, or CIRCLE-Seq. In some embodiments, the ratios of on-target editing:off-target editing relies on the use of EndoV-Seq.

The term “effective amount,” as used herein, refers to an amount of a biologically active agent that is sufficient to elicit a desired biological response. For example, in some embodiments, an effective amount of a composition may refer to the amount of the composition that is sufficient to edit a target site of a nucleotide sequence, e.g. a genome. In some embodiments, an effective amount of a composition provided herein, e.g., of a composition comprising a nuclease-inactive napDNAbp domain, a deaminase domain, a gRNA, may refer to the amount of the composition that is sufficient to induce editing of a target site specifically bound and edited by the base editor. In some embodiments, an effective amount of a composition provided herein may refer to the amount of the composition sufficient to induce editing having the following characteristics: >50% product purity, <5% indels, and an editing window of 2-8 nucleotides. As will be appreciated by the skilled artisan, the effective amount of an agent, e.g., a composition or a base editor-gRNA complex, may vary depending on various factors as, for example, on the desired biological response, e.g., on the specific allele, genome, or target site to be edited, on the cell or tissue being targeted, and/or on the base editor being used.

The term “evolved base editor” or “evolved base editor variant” refers to a base editor formed as a result of mutagenizing a reference or starting-point base editor. The term refers to embodiments in which the nucleotide modification domain is evolved or a separate domain is evolved. Mutagenizing a reference (or starting-point) base editor may comprise mutagenizing an adenosine deaminase. Amino acid sequence variations may include one or more mutated residues within the amino acid sequence of a reference base editor, e.g., as a result of a change in the nucleotide sequence encoding the base editor that results in a change in the codon at any particular position in the coding sequence, the deletion of one or more amino acids (e.g., a truncated protein), the insertion of one or more amino acids, or any combination of the foregoing. The evolved base editor may include variants in one or more components or domains of the base editor (e.g., mutations introduced into one or more adenosine deaminases).

The term “fusion protein” as used herein refers to a hybrid polypeptide which comprises protein domains from at least two proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “host cell,” as used herein, refers to a cell that can host, replicate, and transfer a phage vector useful for a continuous evolution process as provided herein. In embodiments where the vector is a viral vector, a suitable host cell is a cell that may be infected by the viral vector, can replicate it, and can package it into viral particles that can infect fresh host cells. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the disclosure is not limited in this respect. In some embodiments, the viral vector is a phage and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, and XL1-Blue MRF′. These strain names are art recognized and the genotype of these strains has been well characterized. It should be understood that the above strains are exemplary only and that the invention is not limited in this respect. The term “fresh,” as used herein interchangeably with the terms “non-infected” or “uninfected” in the context of host cells, refers to a host cell that has not been infected by a viral vector comprising a gene of interest as used in a continuous evolution process provided herein. A fresh host cell can, however, have been infected by a viral vector unrelated to the vector to be evolved or by a vector of the same or a similar type but not carrying the gene of interest.

In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, will, of course, depend on the viral vector employed, and suitable host cell/viral vector combinations will be readily apparent to those of skill in the art.

In some PACE embodiments, for example, in embodiments employing an M13 selection phage, the host cells are E. coli cells expressing the Fertility factor, also commonly referred to as the F factor, sex factor, or F-plasmid. The F-factor is a bacterial DNA sequence that allows a bacterium to produce a sex pilus necessary for conjugation and is essential for the infection of E. coli cells with certain phage, for example, with M13 phage. For example, in some embodiments, the host cells for M13-PACE are of the genotype F′proA⁺B⁺ Δ(lacIZY) zzf::Tn10(TetR)/endA1 recA1 galE15 galK16 nupG rpsL ΔlacIZYA araD139 Δ(ara,leu)7697 mcrA Δ(mrr-hsdRMS-mcrBC) proBA::pir116λ⁻.

The term “lagoon,” as used herein, refers to a vessel through which a flow of host cells is directed. When used for a continuous evolution process as provided herein, a lagoon typically holds a population of host cells and a population of viral vectors replicating within the host cell population, wherein the lagoon comprises an outflow through which host cells are removed from the lagoon and an inflow through which fresh host cells are introduced into the lagoon, thus replenishing the host cell population. In some embodiments, the flow of cells through the lagoon is regulated to result in an essentially constant number of host cells within the lagoon. In some embodiments, the flow of cells through the lagoon is regulated to result in an essentially constant number of fresh host cells within the lagoon.

The term “linker,” as used herein, refers to a chemical group or a molecule linking two molecules or domains, e.g., dCas9 and a deaminase. Typically, the linker is positioned between, or flanked by, two groups, molecules, or other domains and connected to each one via a covalent bond, thus connecting the two. In some embodiments, the linker is an amino acid or a plurality of amino acids (e.g. a peptide or protein). In some embodiments, the linker is an organic molecule, group, polymer, or chemical domain. Chemical groups include, but are not limited to, disulfide, hydrazone, and azide domains. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, the linker is an XTEN linker. In some embodiments, the linker is a 32-amino acid linker. In other embodiments, the linker is a 30-, 31-, 33- or 34-amino acid linker.

As used herein, the term “low toxicity” refers to the maintenance of a viability above 60% in a population of cells following application of a base editing method or administration of a composition disclosed herein. The term may also refer to prevention of apoptosis (cell death) in a population of cells of more than 40%. For instance, a genome editing method that leads to less than 30% (e.g., 25%, 20%, 15%, 10%, or 5%) cell death exhibits low toxicity. Cell toxicity may be assessed by an appropriate staining assay, e.g., Annexin V and propidium iodide staining assays, and subsequent flow cytometry (e.g. FACS).

The term “mutation,” as used herein, refers to a substitution of a residue within a sequence, e.g. a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is a result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome, which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. Alternatively, the mutation could lead to overexpression of one or more genes involved in control of the cell cycle, thus leading to uncontrolled cell division and hence to cancer. Because of their nature, gain-of-function mutations are usually dominant.

The term “mutagenesis plasmid,” as used herein, refers to a plasmid comprising a gene encoding a gene product that acts as a mutagen. In some embodiments, the gene encodes a DNA polymerase lacking a proofreading capability. In some embodiments, the gene is a gene involved in the bacterial SOS stress response, for example, a UmuC, UmuD, or RecA gene. In some embodiments, the gene is a GATC methylase gene, for example, a deoxyadenosine methylase (dam methylase) gene. In some embodiments, the gene is involved in binding of hemimethylated GATC sequences, for example, a seqA gene. In some embodiments, the gene is involved with repression of mutagenic nucleobase export, for example emrR. Mutagenesis plasmids (also referred to as mutagenesis constructs) are described, for example by International Patent Application, PCT/US2016/027795, filed Apr. 16, 2016, published as WO2016/168631, on Oct. 20, 2016, the entire contents of which are incorporated herein by reference.

The terms “non-naturally occurring” or “engineered” are used interchangeably and indicate the involvement of the hand of man. These terms, when referring to nucleic acid molecules or polypeptides (e.g., deaminases) mean that the nucleic acid molecule or the polypeptide is at least substantially free from at least one other component with which they are naturally associated in nature and/or as found in nature (e.g., an amino acid sequence not found in nature).

The term “nucleic acid,” as used herein, refers to RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g. a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids may be purified from natural sources, produced using recombinant expression systems and optionally purified, chemically synthesized, etc. Where appropriate, e.g. in the case of chemically synthesized molecules, nucleic acids may comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, inosinedenosine, 8-oxoguanosine, 0(6)-methylguanine, and 2-thiocytidine); chemically modified bases; biologically modified bases (e.g., methylated bases, such as 2′-O-methylated bases); intercalated bases; modified sugars (e.g., 2′-fluororibose, ribose, 2′-deoxyribose, arabinose, and hexose); and/or modified phosphate groups (e.g., phosphorothioate and 5′-N-phosphoramidite linkages).

As used herein to modify guide RNA molecules, the term “backbone” refers to the component of the guide RNA that comprises the core region, also known as the crRNA/tracrRNA. The backbone is separate from the guide sequence, or spacer, region of the guide RNA, which has complementarity to a protospacer of a nucleic acid molecule.

The term “nucleic acid programmable DNA binding protein (napDNAbp)” refers to any protein that may associate (e.g., form a complex) with one or more nucleic acid molecules (i.e., which may broadly be referred to as a “napDNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napDNAbp embraces CRISPR-Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a (e.g., LbCas12a, AsCas12a, CeCas12a and MbCas12a), Cas12b, Cas12c, Cas12d, Cas12g, Cas12h, Cas12i, Cas13d, Cas14, Argonaute (Ago), nCas9, xCas9, SpCas9-NG, Cas9-KKH, SmacCas9, Spy-macCas9, an SpCas9-NRRH, an SpCas9-NRCH, and an SpCas9-NRTH, circularly permuted Cas9 domains such as CP1012, CP1028, CP1041, CP1249, and CP1300, and combinations of Cas9 domains such as an CP1041 and an SpCas-NG (SpCas9-NG-CP1041), and an SpCas9-VRQR and an SpCas9-NG (SpCas9-NG-VRQR). Additional napDNAbp Cas equivalents include Cas3 and CasΦ. Additional Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34(7):768-73, which is incorporated herein by reference.

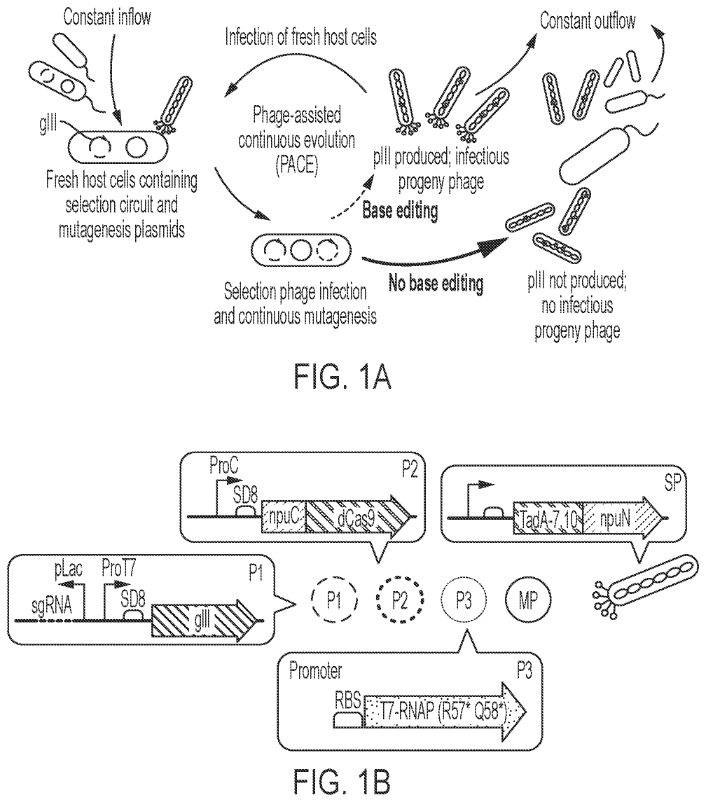

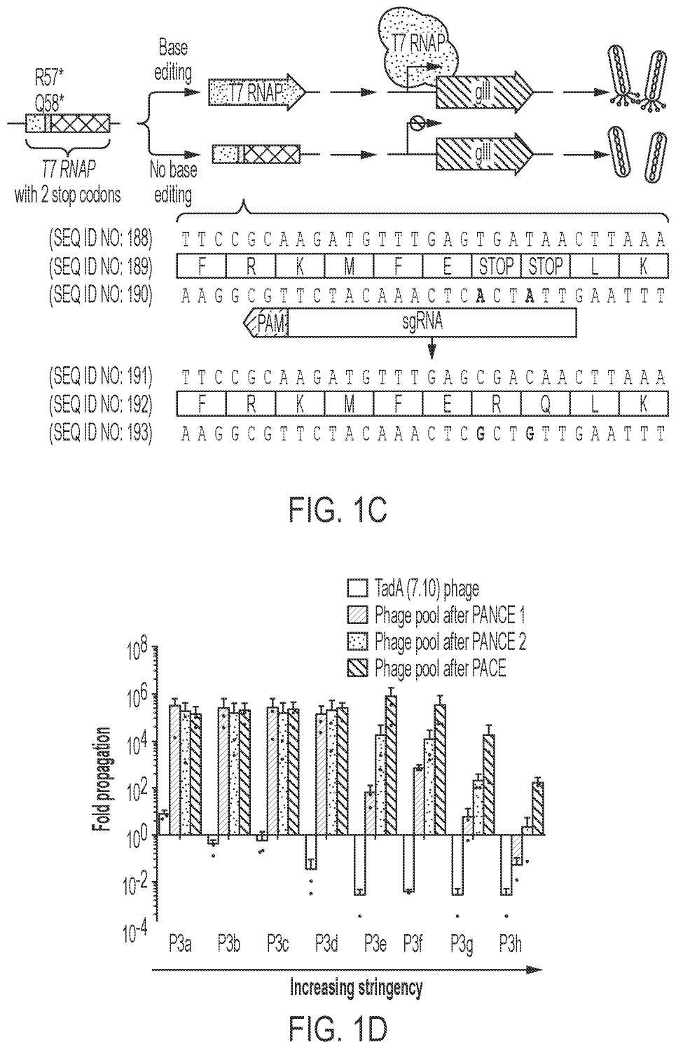

In some embodiments, the napDNAbp is a RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and International Patent Application No. PCT/US2014/054247, filed Sep. 6, 2013, published as WO 2015/035136, and entitled “Delivery System For Functional Nucleases,” the entire contents of each are herein incorporated by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J. et al., Proc. Natl. Acad. Sci. U.S.A. 98:4658-4663(2001); “CRISPR RNA maturation by trans-encoded small RNA and host factor RNase III.” Deltcheva E. et al., Nature 471:602-607(2011); and “A programmable dual-RNA-guided DNA endonuclease in adaptive bacterial immunity.” Jinek M. et al., Science 337:816-821(2012), the entire contents of each of which are incorporated herein by reference.

The napDNAbp nucleases (e.g., Cas9) use RNA:DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

The term “napDNAbp-programming nucleic acid molecule” or equivalently “guide sequence” refers the one or more nucleic acid molecules which associate with and direct or otherwise program a napDNAbp protein to localize to a specific target nucleotide sequence (e.g., a gene locus of a genome) that is complementary to the one or more nucleic acid molecules (or a portion or region thereof) associated with the protein, thereby causing the napDNAbp protein to bind to the nucleotide sequence at the specific target site. A non-limiting example is a guide RNA of a Cas protein of a CRISPR-Cas genome editing system.

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences may be of any size and composition, for example more than 25, 25, 15, 12, 10, 8, 7, 6, 5, or 4 amino acids, but will preferably comprise at least a four to eight amino acid sequence known to function as a nuclear localization signal (NLS).

The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; International PCT Application, PCT/US2016/027795, filed Apr. 15, 2016, published as WO 2016/168631 on Oct. 20, 2016; and International Patent Publication WO 2019/023680, published Jan. 31, 2019, each of which is incorporated herein by reference.

The term “phage-assisted non-continuous evolution (PANCE),” as used herein, refers to non-continuous evolution that employs phage as viral vectors. The general concept of PANCE technology has been described, for example, in Suzuki T. et al., Crystal structures reveal an elusive functional domain of pyrrolysyl-tRNA synthetase, Nat Chem Biol. 13(12): 1261-1266 (2017), incorporated herein by reference in its entirety. Briefly, PANCE is a simplified technique for rapid in vivo directed evolution using serial flask transfers of evolving ‘selection phage’ (SP), which contain a gene of interest to be evolved, across fresh E. coli host cells, thereby allowing genes inside the host E. coli to be held constant while genes contained in the SP continuously evolve. Following phage growth, an aliquot of infected cells is used to transfect a subsequent flask containing host E. coli. This process is continued until the desired phenotype is evolved, for as many transfers as required. Serial flask transfers have long served as a widely-accessible approach for laboratory evolution of microbes, and, more recently, analogous approaches have been developed for bacteriophage evolution. The PANCE system features lower stringency than the PACE system.

The term “promoter” is art-recognized and refers to a nucleic acid molecule with a sequence recognized by the cellular transcription machinery and able to initiate transcription of a downstream gene. A promoter may be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect. In various embodiments, the disclosure provides vectors with appropriate promoters for driving expression of the nucleic acid sequences encoding the base editors (or one or more individual components thereof).

The term “selection phage,” as used herein interchangeably with the term “selection plasmid” and in the context of phage-assisted continuous evolution (PACE) systems, refers to a modified phage that comprises a nucleic acid sequence encoding an adenosine deaminase to be evolved, and lacks a full-length gene encoding a protein required for the generation of infectious phage particles. For example, some M13 selection phages provided herein comprise a nucleic acid sequence encoding a gene to be evolved, e.g., under the control of an M13 promoter, and lack all or part of a phage gene encoding a protein required for the generation of infectious phage particles, e.g., gI, gII, gIII, gIV, gV, gVI, gVII, gVIII, gIX, or gX, or any combination thereof. For example, some M13 selection phages provided herein comprise a nucleic acid sequence encoding an adenosine deaminase to be evolved, e.g., under the control of an M13 promoter, and lack all or part of a gene encoding a protein required for the generation of infective phage particles, e.g., the gIII gene encoding the pIII protein.

The term “subject,” as used herein, refers to an individual organism, for example, an individual mammal. In some embodiments, the subject is a human. In some embodiments, the subject is a non-human mammal. In some embodiments, the subject or organism is a non-human primate. In some embodiments, the subject or organism is a rodent. In some embodiments, the subject or organism is a sheep, a goat, cattle, a cat, or a dog. In some embodiments, the subject or organism is a vertebrate, an amphibian, a reptile, a fish, an insect, a fly, or a nematode. In some embodiments, the subject or organism is a research animal. In some embodiments, the subject or organism is a domesticated animal. In some embodiments, the subject or organism is a plant. In some embodiments, the subject or organism is genetically engineered, e.g. a genetically engineered non-human subject. The subject or organism may be of either sex and at any stage of development.

The term “target site” refers to a sequence within a nucleic acid molecule that is edited by a base editor. The target site further refers to the sequence within a nucleic acid molecule to which a complex of the base editor and gRNA binds.

The terms “treatment,” “treat,” and “treating,” refer to a clinical intervention aimed to reverse, alleviate, delay the onset of, or inhibit the progress of a disease, disorder, or condition, or one or more symptoms thereof, as described herein. As used herein, the terms “treatment,” “treat,” and “treating” refer to a clinical intervention aimed to reverse, alleviate, delay the onset of, or inhibit the progress of a disease, disorder, or condition, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their prevention or recurrence.

The term “target site” refers to a sequence within a nucleic acid molecule that is deaminated by a deaminase or a base editor comprising a deaminase (e.g., an adenine base editor provided herein).

As used herein, e.g. for the purposes of reporting a specific number of loci, the terms “unique loci” and “unique genomic loci” refer to distinct genomic sequences (e.g. distinct coding sequences) wherein all copies of a distinct sequence in the genome are collectively counted (or reported) only once; in contrast, each copy of a “non-unique locus” or “repetitive element” is counted for purposes of reporting a specific number of loci.

As used herein, the term “variant” refers to a protein having characteristics that deviate from what occurs in nature that retains at least one functional i.e. binding, interaction, or enzymatic ability and/or therapeutic property thereof. A “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant of Cas9 may comprise a Cas9 that has one or more changes in amino acid residues as compared to a wild type Cas9 amino acid sequence. As another example, a variant of a deaminase may comprise a deaminase that has one or more changes in amino acid residues as compared to a wild type deaminase amino acid sequence, e.g. following ancestral sequence reconstruction of the deaminase. These changes include chemical modifications, including substitutions of different amino acid residues truncations, covalent additions (e.g. of a tag), and any other mutations. This term also embraces fragments of a wild type protein.

The level or degree of which the property is retained may be reduced relative to the wild type protein but is typically the same or similar in kind. Generally, variants are overall very similar, and in many regions, identical to the amino acid sequence of the protein described herein. A skilled artisan will appreciate how to make and use variants that maintain all, or at least some, of a functional ability or property.

The variant proteins may comprise, or alternatively consist of, an amino acid sequence which is at least 80%, 85%, 90%, 95%, 96%, 97%, 98%, 99%, or 100%, identical to, for example, the amino acid sequence of a wild-type protein, or any protein provided herein (e.g., the adenosine deaminase domain of an adenine base editor). Further polypeptides encompassed by the invention are polypeptides encoded by polynucleotides which hybridize to the complement of a nucleic acid molecule encoding a protein such as a napDNAbp under stringent hybridization conditions (e.g. hybridization to filter bound DNA in 6×Sodium chloride/Sodium citrate (SSC) at about 45 degrees Celsius, followed by one or more washes in 0.2.times.SSC, 0.1% SDS at about 50-65 degrees Celsius), under highly stringent conditions (e.g. hybridization to filter bound DNA in 6×sodium chloride/Sodium citrate (SSC) at about 45 degrees Celsius, followed by one or more washes in 0.1×SSC, 0.2% SDS at about 68 degrees Celsius), or under other stringent hybridization conditions which are known to those of skill in the art (see, for example, Ausubel, F. M. et al., eds., 1989 Current Protocol in Molecular Biology, Green publishing associates, Inc., and John Wiley & Sons Inc., New York, at pp. 6.3.1-6.3.6 and 2.10.3).

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence of a protein such as a napDNAbp, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

As used herein, the term “wild type” is a term of the art understood by skilled persons and means the typical form of an organism, strain, gene, or characteristic as it occurs in nature as distinguished from mutant or variant forms.

These and other exemplary substituents are described in more detail in the Detailed Description, Examples, Figures, and Claims. The invention is not intended to be limited in any manner by the above exemplary listing of substituents.

### DETAILED DESCRIPTION OF THE INVENTION

The present disclosure provides adenine base editors that comprise an adenosine deaminase domain (e.g., a variant of an adenosine deaminase that deaminates deoxyadenosine in DNA as described herein) and a napDNAbp domain (e.g., a Cas9 protein) capable of binding to a specific nucleotide sequence, wherein the adenosine deaminase variants provide the base editor with lower off-target effects while maintaining editing efficiencies of existing ABEs. The deamination of an adenosine by an adenosine deaminase may lead to a point mutation from adenine (A) to guanine (G), a process referred to herein as nucleic acid editing. For example, the adenosine may be converted to an inosine residue. Within the constraints of a DNA polymerase active site, inosine pairs most stably with C and therefore is read or replicated by the cell's replication machinery as a guanine (G). Such base editors are useful inter alia for targeted editing of nucleic acid sequences. Such base editors may be used for targeted editing of DNA in vitro, e.g., for the generation of mutant cells or animals. Such base editors may be used for the introduction of targeted mutations in the cell of a living mammal. Such base editors may also be used for the introduction of targeted mutations for the correction of genetic defects in cells ex vivo, e.g., in cells obtained from a subject that are subsequently re-introduced into the same or another subject, or for multiplexed editing of a genome. And these base editors may be used for the introduction of targeted mutations in vivo, e.g., the correction of genetic defects or the introduction of deactivating mutations in disease-associated genes in a subject, or for multiplexed editing of a genome. The adenine base editors described herein may be utilized for the targeted editing of G to A mutations (e.g., targeted genome editing). The invention provides deaminases, base editors, nucleic acids, vectors, cells, compositions, methods, kits, and uses that utilize the deaminases and base editors provided herein.

In some embodiments, the present disclosure provides base editors having adenosine deaminase domains that are mutated (e.g. evolved to have mutations) that enable the deaminase domain to have improved activity when used with Cas homologs (e.g., homologs other than SpCas9). Accordingly, the present disclosure provides variants of adenosine deaminases (e.g., variants of TadA7.10) engineered from PACE and PANCE methodologies. These variants include ABE8e, which contains eight additional mutations relative to the TadA7.10 deaminase domain (where TadA7.10 contains the mutations W23R, H36L, P48A, R51L, L84F, A106V, D108N, H123Y, S146C, D147Y, R152P, E155V, I156F, and K157N in the ecTadA sequence). This motif increases deaminase activity (kₐₚₚ) 590-fold relative to that of ABE7.10. This evolved variant is broadly compatible with diverse Cas9 or Cas12 homologs, and exhibits improved editing efficiencies when paired with previously incompatible Cas9 or Cas12 homologs. For instance, disclosed adenosine deaminase variants such as TadA8e exhibit higher editing efficiencies when paired in a base editor with certain Cas9 variants, such as circularly permuted variants CP1041 and CP1028, than exhibited by the TadA7.10 deaminase.

ABE8e shows substantially increased editing efficiency relative to ABE7.10 in various base editing contexts. ABE8e also has increased processivity compared to ABE7.10, i.e., it enhances the frequency of alleles with multiple base conversions between 11- and 17-fold relative to ABE7.10. On-target:off-target DNA editing ratios with ABE8e are similar to those of ABE7.10. And, although ABE8e shows increased off-target RNA and DNA editing relative to ABE7.10, these off-target effects were reduced by introducing a V106W mutation into the TadA-8e domain.

The present disclosure provides additional variants of ABE7.10 that contain mutations in the TadA deaminase domain of the ABE. These evolved variants are also broadly compatible with diverse Cas9 and Cas12 homologs and exhibit improved editing efficiencies (up to 58-fold increases in average editing) when paired with previously incompatible Cas9 or Cas12 homologs. In certain embodiments, the base editors have increased processivity.

ABE8e and other variants exhibit modest increases in transcriptome-wide RNA off-target editing compared to ABE7.10, which is diminished by introducing mutations in the TadA-8e domain. ABE8e and other variants enable efficient base editing of hemoglobinopathy-protective natural point mutations in a GATA1 binding site in the BCL11A enhancer or in the HBG promoter in human cells, in contrast with canonical ABE7.10 (set forth in SEQ ID NO: 487).

Accordingly, the disclosure provides adenosine deaminase variants. In some embodiments, the adenosine deaminases provided herein comprise an amino acid sequence that comprises substitutions selected from T111X₁, D119X₂, and F149X₃, and further comprises at least one substitution selected from R26X₄, V88X₅, A109X₆, H122X₇, T166X₈, and D167X₉, relative to the amino acid sequence of SEQ ID NO: 315 (TadA7.10), or a corresponding substitution(s) in another adenosine deaminase, wherein X₁ is any amino acid other than T, X₂ is any amino acid other than D, X₃ is any amino acid other than F, X₄ is any amino acid other than R, X₅ is any amino acid other than V, X₆ is any amino acid other than A, X₇ is any amino acid other than H, X₈ is any amino acid other than T, and X₉ is any amino acid other than D. In some embodiments, the disclosure provides adenosine deaminase that further comprises a Y167X₁₀ relative to the amino acid sequence of SEQ ID NO: 315, or a corresponding substitution in another adenosine deaminase, wherein X₁₀ is any amino acid other than Y. In particular embodiments, X₁₀ is D. In some embodiments, the adenosine deaminases of the disclosure may further comprise at least one substitution selected from K20X₁₁, R21X₁₂, V82X₁₃, and V106X₁₃ relative to the amino acid sequence of SEQ ID NO: 315, or a corresponding substitution(s) in another adenosine deaminase, wherein X₁₁ is any amino acid other than K, X₁₂ is any amino acid other than R, X₁₃ is any amino acid other than V, and X₁₄ is any amino acid other than V.

In some embodiments, the described adenosine deaminases comprise T111R, D119N, and F149Y substitutions, and further comprises at least one substitution selected from R26C, V88A, A109S, H122N, T1661, and D167N, in the amino acid sequence corresponding to TadA7.10 (SEQ ID NO: 315), or corresponding substitutions in another adenosine deaminase. In some embodiments, the disclosed adenosine deaminase comprises the substitutions A109S, T111R, D119N, H122N, Y147D, F149Y, T1661 and D167N in its amino acid sequence. In certain embodiments, the adenosine deaminases comprise the substitutions V106W, A109S, T111R, D119N, H122N, Y147D, F149Y, T1661 and D167N. In an exemplary embodiment, the adenosine deaminase comprises TadA-8e (SEQ ID NO: 433). In another exemplary embodiment, the adenosine deaminase comprises TadA-8e (V106W) (SEQ ID NO: 434).

In some aspects, the disclosure provides base editors comprising one or more adenosine deaminase variants disclosed herein and a napDNAbp domain. In some embodiments, the napDNAbp domain comprises a Cas homolog. The napDNAbp domain may be selected from a Cas9, a Cas9n, a dCas9, a CasX, a CasY, a C2c1, a C2c2, a C2c3, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, an SpCas9-NG-CP1041, an SpCas9-NG-VRQR, an LbCas12a, an AsCas12a, a Cas9-KKH, a circularly permuted Cas9, an Argonaute (Ago) domain, a SmacCas9, a Spy-macCas9, an SpCas9-VRQR, an SpCas9-NRRH, an SpaCas9-NRTH, an SpCas9-NRCH. In certain embodiments, the napDNAbp domain comprises or is a Cas9 domain or a Cas12a domain derived from S. pyogenes or S. aureus. In some embodiments, the napDNAbp domain comprises a nuclease dead Cas9 (dCas9) domain, a Cas9 nickase (nCas9) domain, or a nuclease active Cas9 domain.

Exemplary napDNAbp domains include, but are not limited to S. pyogenes Cas9 nickase (SpCas9n) and S. aureus Cas9 nickase (SaCas9n). Additional exemplary napDNAbp domains include S. aureus Cas9-KKH (SaCas9-KKH), LbCas12a, enAsCas12a (an engineered AsCas12a recently reported by Joung et al.⁹), SpCas9-NG, SpCas9-VRQR, SpCas9-NG-CP1041, SpCas9-NG-VRQR, SpCas9-NRCH, CP1028-SpCas9, and CP1041-SpCas9. In some embodiments, the napDNAbp domain comprises a Cas9 nickase (nCas9) domain. In some embodiments, the napDNAbp domain comprises an SpCas9n domain, e.g., a SpCas9n having the amino acid sequence set forth as SEQ ID NO: 365 or 370. In certain embodiments, the napDNAbp domain of any of the disclosed base editors is a SaCas9n, e.g., an SpCas9n having the amino acid sequence set forth as SEQ ID NO: 438. In certain embodiments, the napDNAbp domain of any of the disclosed base editors is an SpCas9-NRCH, e.g., an SpCas9-NRCH having the amino acid sequence set forth as SEQ ID NO: 436. In certain embodiments, the napDNAbp domain of any of the disclosed base editors is an LbCas12a, e.g., a catalytically inactive or “dead” LbCas12a. In certain embodiments, the napDNAbp domain of any of the disclosed base editors is an AsCas12a, e.g., an enAsCas12a. In certain embodiments, the napDNAbp domain of any of the disclosed base editors is a cicular permuted variant of SpCas9, e.g., a CP1028-SpCas9 or a CP1041-SpCas9. In certain embodiments, the napDNAbp domain of any of the disclosed base editors is an evolved SpCas9, e.g., an SpCas9-NG. In certain embodiments, the napDNAbp domain of any of the disclosed base editors is an SpCas9-NG-CP1041. In certain embodiments, the napDNAbp domain of any of the disclosed based editors is SpCas9-NG-VRQR.

In some embodiments, the napDNAbp comprises an amino acid sequence that is at least 90%, at least 95%, at least 98%, or at least 99% identical to any one of SEQ ID NOs: 435-442, 463 and 464.

In some embodiments, the napDNAbp is selected from SEQ ID NOs: 435-442, 463 and 464. In some embodiments, the napDNAbp is selected from SEQ ID NOs: 365, 370, 406 and 407.

Further provided herein are methods of contacting any of the disclosed adenine base editors with a nucleic acid molecule, e.g., a nucleic acid molecule (e.g., DNA) comprising a target sequence. In some embodiments of the disclosed methods, low off-target DNA and/or RNA editing effects are observed. In some embodiments, the nucleic acid molecule comprises a DNA, e.g., a single-stranded DNA or a double-stranded DNA. The target sequence of the nucleic acid molecule may comprise a target nucleobase pair containing an adenine (A). The target sequence may be comprised within a genome, e.g., a human genome. The target sequence may comprise a sequence, e.g., a target sequence with point mutation, associated with a disease or disorder. The target sequence with a point mutation may be associated with sickle cell disease. The target sequence with a point mutation may be associated with Glycogen storage disease type 1A or Stargardt macular dystrophy.

In some aspects, the present disclosure provides compositions comprising the adenine base editors with reduced RNA editing effects as described herein and one or more guide RNAs, e.g., a single-guide RNA (“sgRNA”). In addition, the present disclosure provides for nucleic acid molecules encoding and/or expressing the adenine base editors as described herein, as well as expression vectors or constructs for expressing the adenine base editors described herein and a gRNA, host cells comprising said nucleic acid molecules and expression vectors, and optionally one or more gRNAs, and compositions for delivering and/or administering nucleic acid-based embodiments described herein.

In some embodiments, the target nucleotide sequence is a DNA sequence in a genome, e.g., a eukaryotic genome. In certain embodiments, the target nucleotide sequence is in a mammalian (e.g., a human) genome. In certain embodiments, the target nucleotide sequence is in a human genome. In other embodiments, the target nucleotide sequence is in the genome of a rodent, such as a mouse or a rat. In other embodiments, the target nucleotide sequence is in the genome of a domesticated animal, such as a horse, cat, dog, or rabbit. In some embodiments, the target nucleotide sequence is in the genome of a research animal. In some embodiments, the target nucleotide sequence is in the genome of a genetically engineered non-human subject. In some embodiments, the target nucleotide sequence is in the genome of a plant. In some embodiments, the target nucleotide sequence is in the genome of a microorganism, such as a bacteria.

Without wishing to be bound by any particular theory, the adenine base editors described herein induce edits in nucleic acid substrates by use of TadA variants to deaminate A bases, causing A to G mutations via inosine formation. Inosine preferentially hydrogen bonds with C, resulting in an A to G mutation during DNA replication. When covalently tethered to a nucleic acid programmable DNA binding protein, the adenosine deaminase is localized to a target of interest and catalyzes A to G mutations in the DNA substrate.

In some embodiments, this editor may be used to target and revert single nucleotide polymorphisms (SNPs) in disease-relevant genes, which require A to G reversion. In some embodiments, any of the disclosed editors are used to target and revert an A to G mutation associated with sickle cell disease, Glycogen storage disease type 1A, or Stargardt macular dystrophy. The ABE editor can also be used to target and revert single nucleotide polymorphisms (SNPs) in disease-relevant genes, which require T to C reversion by mutating the A, opposite of the T, to a G. The T may then be replaced with a C, for example, by base excision repair mechanisms, or may be changed in subsequent rounds of DNA replication. For example, a reversion of −198T to C, or a reversion of −175T to C, in the promoter driving HBG1 and HBG2 gene expression by any of the disclosed base editors may result in increased expression of HBG1 and HBG2, and correction of the sickle cell disease phenotype. In other embodiments, the ABE editor is used to target and convert (but not revert) a mutant T to a mutant C (by mutating the A opposite of the T), wherein the SNP with a mutant C encodes a non-pathogenic variant. In some embodiments, this variant is found in nature. Such a strategy is used in connection with use of any of the disclosed base editors to convert a mutant T in an HBB allel—an SNP associated with sickle cell disease—to a variant known as the Makassar allele that does not result in a disease phenotype. Thus, the adenine base editors described herein may deaminate the A nucleobase to yield a nucleotide sequence that is not associated with a disease or disorder.

In some aspects, the disclosure provides complexes comprising the adenine base editors as described herein and one or more guide RNAs, e.g., a single-guide RNA (“sgRNA”), as well as compositions comprising any of these complexes. In addition, the present disclosure provides for nucleic acid molecules encoding and/or expressing the base editors as described herein, as well as expression vectors and constructs for expressing the base editors described herein and/or a gRNA (e.g., AAV vectors), host cells comprising any of said nucleic acid molecules and expression vectors and optionally vectors encoding one or more gRNAs, host cells comprising any of said base editors and optionally one or more gRNAs, and methods for delivering and/or administering nucleic acid-based embodiments described herein. In particular, the disclosure provides improved methods of delivery of the disclosed base editors, e.g., to a subject. Delivery of the disclosed ABE variants as RNPs, rather than DNA plasmids, typically increases on-target:off-target DNA editing ratios. Delivery of the disclosed ABE variants as mRNA molecules (e.g., using electroporation) may increase editing efficiencies.

Still further, the present disclosure provides for methods of creating the base editors described herein, as well as methods of using the base editors or nucleic acid molecules encoding any of these base editors in applications including editing a nucleic acid molecule, e.g., a genome. In certain embodiments, methods of engineering the base editors provided herein involve a phage-assisted continuous evolution (PACE) system or non-continuous system (e.g., PANCE), which may be utilized to evolve one or more components of a base editor (e.g., a deaminase domain). In certain embodiments, following the successful evolution of one or more components of the base editor (e.g., a deaminase domain), methods of making the base editors comprise recombinant protein expression methodologies and techniques known to those of skill in the art. Exemplary base editors are made by fusing or associating the adenosine deaminase domain to any of a variety of napDNAbp domains disclosed herein, such as a Cas9 domain.

The domains of the adenine base editors described herein (e.g., the napDNAbp domain or the adenosine deaminase domain) may be obtained as a result of mutagenizing a reference base editor (or a component or domain thereof) by a directed evolution process, e.g., a continuous evolution method (e.g., PACE) or a non-continuous evolution method (e.g., PANCE or other discrete plate-based selections). In various embodiments, the disclosure provides an adenine base editor that has one or more amino acid variations introduced into its amino acid sequence relative to the amino acid sequence of the reference base editor. The base editor may include variants in one or more components or domains of the base editor (e.g., variants introduced into a adenosine deaminase domain, or a variant introduced into both of these domains).

The nucleotide modification domain may be engineered in any way known to those of skill in the art. For example, the nucleotide modification domain may be evolved from a reference protein and evolved using PACE, PANCE, or other plate-based evolution methods to obtain a DNA modifying version of the nucleotide modification domain, which can then be used in the base editors described herein. For example, the disclosed adenosine deaminase variants may be at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the reference enzyme. In some embodiments, the adenosine deaminase variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to a reference adenosine deaminase.

### Adenosine Deaminase Domains

The disclosure provides base editors that comprise an adenosine deaminase domain. In some aspects, any of the disclosed base editors are capable of deaminating adenosine in a nucleic acid sequence (e.g., DNA or RNA). As one example, any of the base editors provided herein may be base editors (e.g., adenine base editors). In some embodiments, the disclosed adenosine deaminases are variants of known adenosine deaminase TadA7.10, which comprises the following mutations as compared to wild-type ecTadA (SEQ ID NO: 325): W23R, H36L, P48A, R51L, L84F, A106V, D108N, H123Y, S146C, D147Y, R152P, E155V, I156F, and K157N. In some embodiments, the disclosed adenosine deaminases are variants of a TadA derived from a species other than E. coli, such as Staphylococcus aureus, Salmonella typhi, Shewanella putrefaciens, Haemophilus influenzae, Caulobacter crescentus, or Bacillus subtilis.

In various embodiments, the adenosine deaminases of the disclosed base editors hydrolytically deaminate a targeted adenosine in a nucleic acid of interest to an inosine, which is read as a guanosine (G) by DNA polymerase enzymes.

Exemplary, non-limiting, embodiments of adenosine deaminases are provided herein. In some embodiments, the adenosine deaminase domain of any of the disclosed base editors comprises a single adenosine deaminase, or a monomer. In some embodiments, the adenosine deaminase domain comprises 2, 3, 4 or 5 adenosine deaminases. In some embodiments, the adenosine deaminase domain comprises two adenosine deaminases, or a dimer. In some embodiments, the deaminase domain comprises a dimer of an engineered (or evolved) deaminase and a wild-type deaminase, such as a wild-type E. coli-derived deaminase. It should be appreciated that the mutations provided herein (e.g., mutations in ecTadA) may be applied to adenosine deaminases in other adenosine base editors, for example, those provided in International Publication No. WO 2018/027078, published Aug. 2, 2018; International Publication No. WO 2019/079347 on Apr. 25, 2019; International Application No PCT/US2019/033848, filed May 23, 2019, which published as International Publication No. WO 2019/226953 on Nov. 28, 2019; U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, and International Patent Application No. PCT/US2020/28568, filed Apr. 16, 2020; all of which are incorporated herein by reference in their entireties.

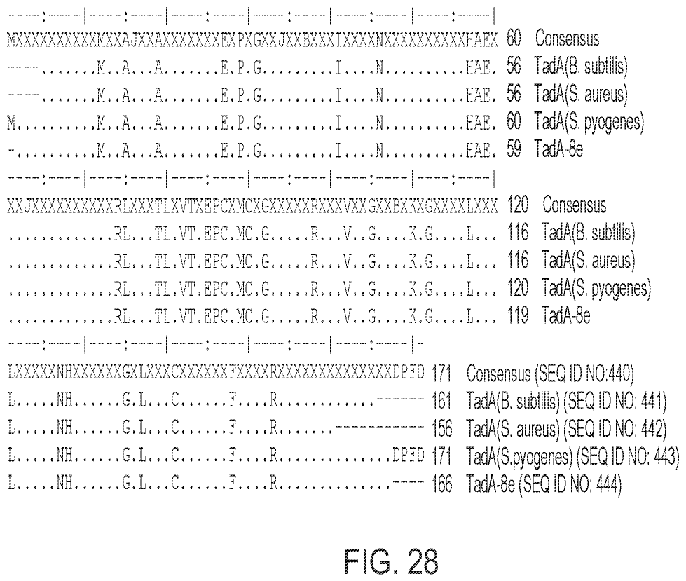

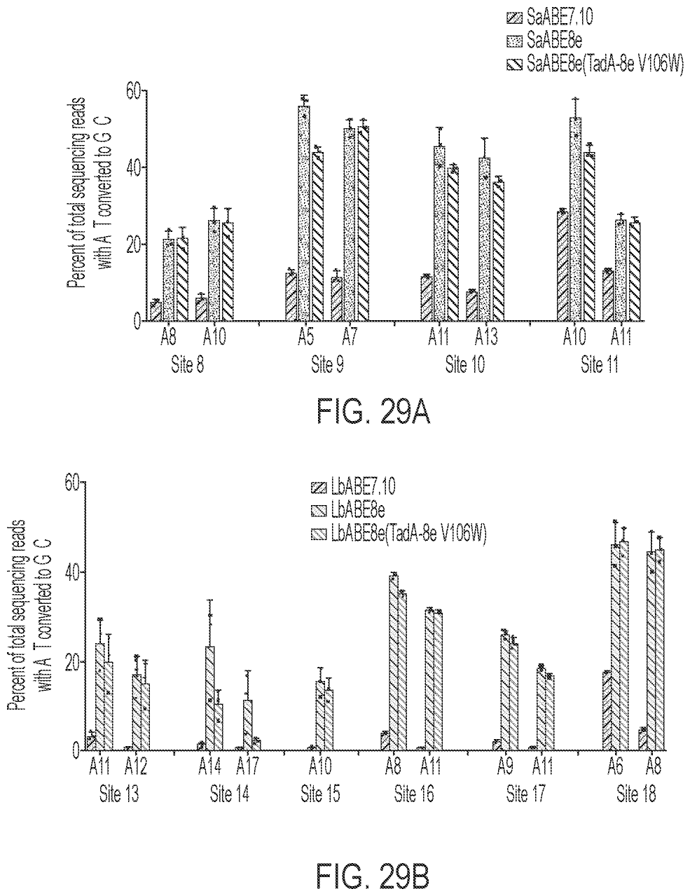

In some embodiments, any of the adenosine deaminases provided herein are capable of deaminating adenine, e.g., deaminating adenine in a deoxyadenosine residue of DNA. The adenosine deaminase may be derived from any suitable organism (e.g., E. coli). In some embodiments, the adenosine deaminase is a naturally-occurring adenosine deaminase that includes one or more mutations corresponding to any of the mutations provided herein (e.g., mutations in ecTadA). One of skill in the art will be able to identify the corresponding residue in any homologous protein and in the respective encoding nucleic acid by methods well known in the art, e.g., by sequence alignment and determination of homologous residues. An amino acid sequence alignment of exemplary TadA deaminases derived from Bacillus subtilis (set forth in full as SEQ ID NO: 318), S. aureus (SEQ ID NO: 317), and S. pyogenes (SEQ ID NO: 448) as compared to the consensus sequence of E. coli TadA is provided as FIG. 28. The amino acid substitutions in (E. coli) TadA-8e, and the homologous mutations in the B. subtilis, S. aureus, and S. pyogenes TadA deaminases, are shown. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is derived from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In some embodiments, the adenosine deaminase domain comprises an adenosine deaminase that comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 324, 325, 433, 434, and 448 which are based on ecTadA, or to any of the adenosine deaminases provided herein. In certain embodiments, the adenosine deaminase comprises an amino acid sequence that is at least 80%, at least 85%, at least 90%, at least 95%, at least 96%, at least 97%, at least 98%, at least 99%, or at least 99.5% identical to the amino acid sequence of TadA7.10 (SEQ ID NO: 315). It should be appreciated that adenosine deaminases provided herein may include one or more mutations (e.g., any of the mutations provided herein). The disclosure provides adenosine deaminases with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 315-325 (e.g., TadA7.10), 433, 434, and 448, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in SEQ ID NOs: 315-325 (e.g., TadA7.10), 433, 434, and 448, or any of the adenosine deaminases provided herein. In some embodiments, the adenosine deaminase comprises a variant of TadA 7.10, whose sequence is set forth as SEQ ID NO: 315.

In some aspects, the disclosure provides an adenosine deaminase comprising an amino acid sequence that comprises substitutions selected from T111X₁, D119X₂, and F149X₃, and further comprises at least one substitution selected from R26X₄, V88X₅, A109X₆, H122X₇, T166X₈, and D167X₉, relative to the amino acid sequence of SEQ ID NO: 315, or a corresponding substitution(s) in another adenosine deaminase, wherein X₁ is any amino acid other than T, X₂ is any amino acid other than D, X₃ is any amino acid other than F, X₄ is any amino acid other than R, X₅ is any amino acid other than V, X₆ is any amino acid other than A, X₇ is any amino acid other than H, X₈ is any amino acid other than T, and X₉ is any amino acid other than D.

In some embodiments, X₁ is a hydrophobic amino acid. In other embodiments, X₁ is a hydrophilic amino acid. In some embodiments, X₁ is a small amino acid. In other embodiments, X₁ is a large amino acid. In some embodiments, X₁ is R. In some embodiments, X₂ is a hydrophobic amino acid, a hydrophilic amino acid, a small amino acid, or a large amino acid. In some embodiments, X₂ is D. In some embodiments, X₃ is a hydrophobic amino acid. In other embodiments, X₃ is a large amino acid. In some embodiments, X₃ is Y.

In some embodiments, X₄ is a hydrophobic amino acid. In some embodiments, X₄ is a small amino acid. In some embodiments, X₄ is C. In some embodiments, X₅ is a hydrophobic amino acid. In some embodiments, X₅ is a small amino acid. In some embodiments, X₅ is A.

In some embodiments, one or more of X₆, X₇, X₈, or X₉ is a hydrophilic amino acid. In some embodiments, one or more of X₆, X₇, X₈, or X₉ is a small amino acid. In some embodiments, X₆ is S. In some embodiments, X₇ is N. In some embodiments, X₈ is I. In some embodiments, X₉ is N.

In some aspects, the disclosure provides adenosine deaminase that further comprises a Y167X₁₀ relative to the amino acid sequence of SEQ ID NO: 315, or a corresponding substitution in another adenosine deaminase, wherein X₁₀ is any amino acid other than Y. In some embodiments, X₁₀ is a hydrophobic amino acid. In other embodiments, X₁₀ is a hydrophilic amino acid. In some embodiments, X₁₀ is a small amino acid. In other embodiments, X₁₀ is a large amino acid. In particular embodiments, X₁₀ is D.

In another embodiment, the adenosine deaminases of the disclosure may further comprise at least one substitution selected from K20X₁₁, R21X₁₂, V82X₁₃, and V106X₁₃ relative to the amino acid sequence of SEQ ID NO: 315, or a corresponding substitution(s) in another adenosine deaminase, wherein X₁₁ is any amino acid other than K, X₁₂ is any amino acid other than R, X₁₃ is any amino acid other than V, and X₁₄ is any amino acid other than V. In some embodiments, one or more of X₁₁, X₁₂, X₁₃, or X₁₄ is a hydrophobic amino acid. In some embodiments, one or more of X₁₁, X₁₂, X₁₃, or X₁₄ is a small amino acid. In some embodiments, X₁₁ is A. In some embodiments, X₁₂ is A. In some embodiments, X₁₃ is G. In some embodiments, X₁₄ is a large amino acid. In some embodiments, X₁₄ is W.

In some embodiments, the adenosine deaminase comprises A109S, T111R, D119N, H122N, F149Y, T1661, and D167N substitutions in SEQ ID NO: 315, or a corresponding mutation in another adenosine deaminase. In some embodiments, the adenosine deaminase comprises R26C, D108W, T111R, D119N, and F149Y substitutions in SEQ ID NO: 315, or a corresponding mutation in another adenosine deaminase. In some embodiments, the adenosine deaminase comprises V88A, D108W, T111R, D119N, and F149Y substitutions in SEQ ID NO: 315, or a corresponding mutation in another adenosine deaminase. In some embodiments, the adenosine deaminase further comprises a Y147D substitution.

In some embodiments, the adenosine deaminase comprises A109S, T111R, D119N, H122N, Y147D, F149Y, T1661, and D167N substitutions in SEQ ID NO: 315, or a corresponding mutation in another adenosine deaminase. In some embodiments, the adenosine deaminase comprises TadA-8e (SEQ ID NO: 433). In some embodiments, the adenosine deaminase comprises A109S, T111R, D119N, H122N, Y147D, F149Y, T1661, and D167N in SEQ ID NO: 315, or a corresponding mutation in another adenosine deaminase. In some embodiments, the adenosine deaminase further comprises at least one substitution in the amino acid sequence of SEQ ID NO: 315 selected from K20A, R21A, V82G, and V106W. In certain embodiments, the adenosine deaminase comprises V106W, A109S, T111R, D119N, H122N, Y147D, F149Y, T1661 and D167N substitutions in SEQ ID NO: 315. In some embodiments, the adenosine deaminase comprises or is TadA-8e(V106W) (SEQ ID NO: 434). It should be appreciated, however, that additional deaminases may similarly be aligned to identify homologous amino acid residues that may be mutated as provided herein.

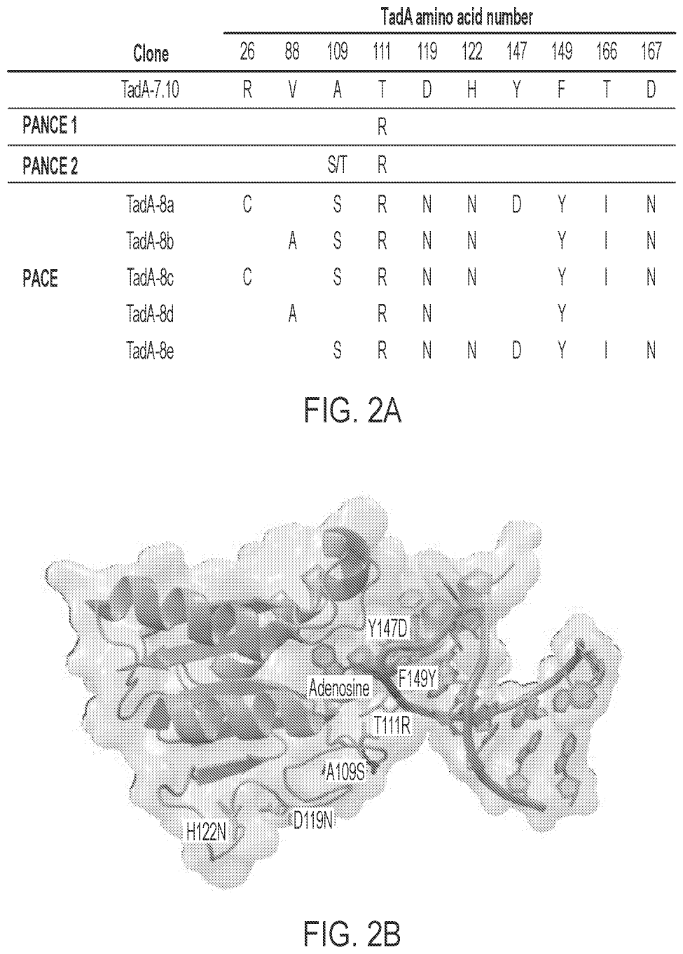

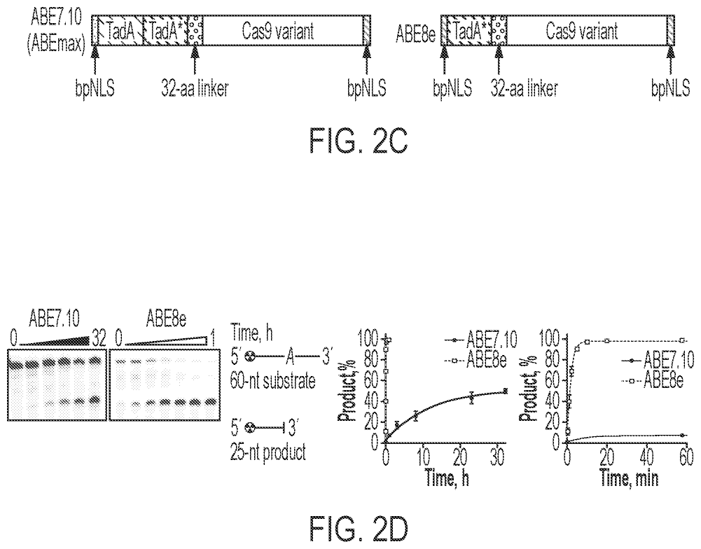

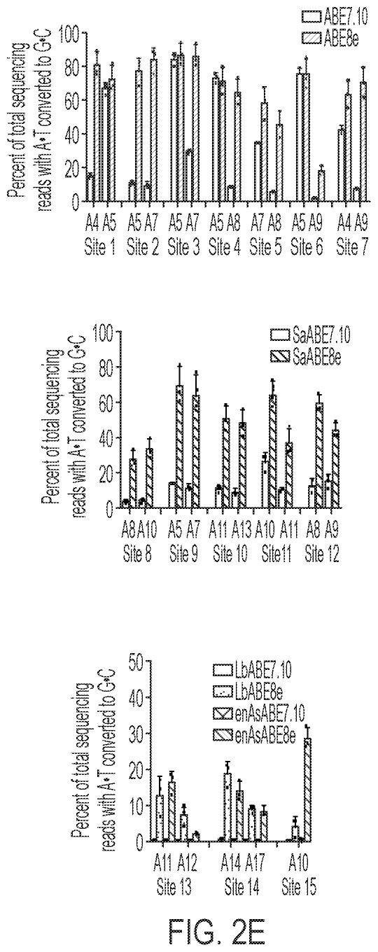

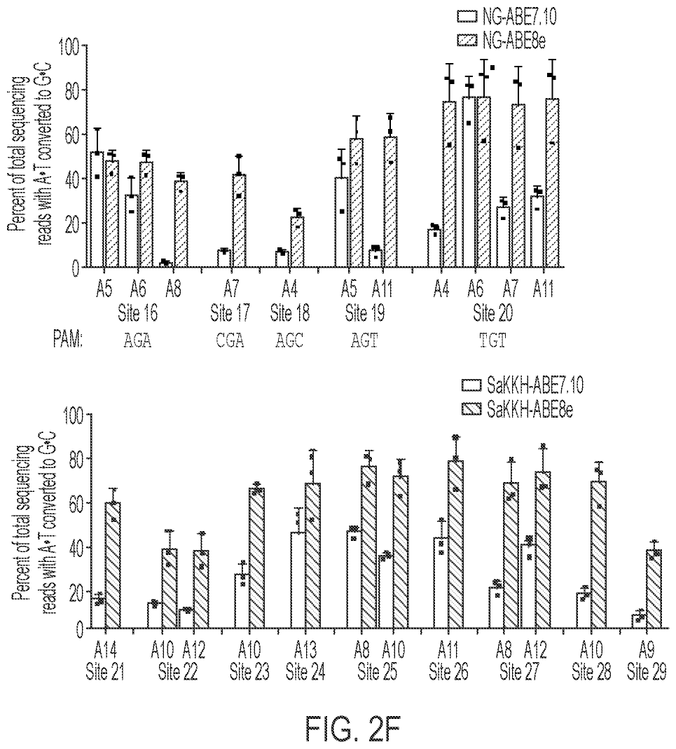

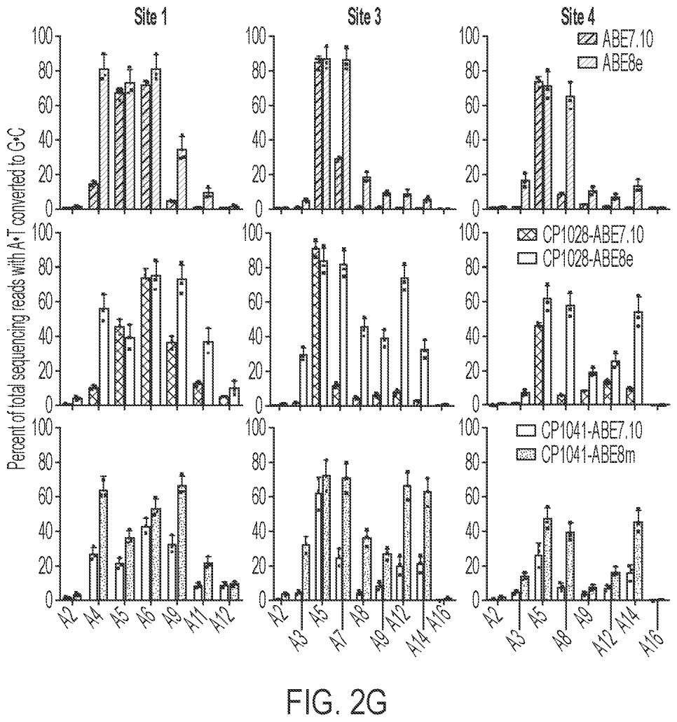

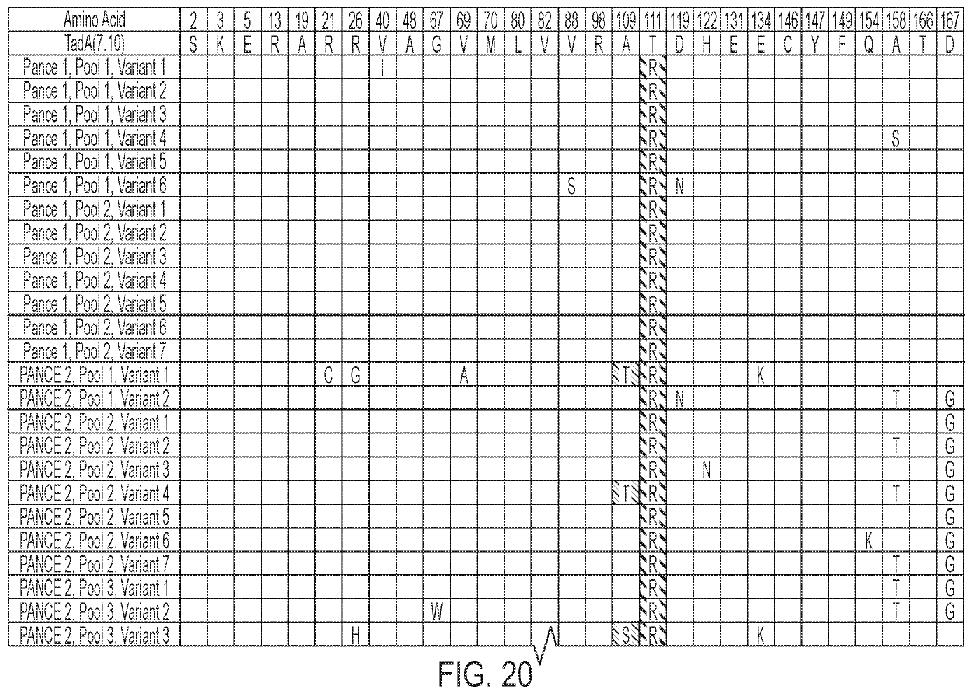

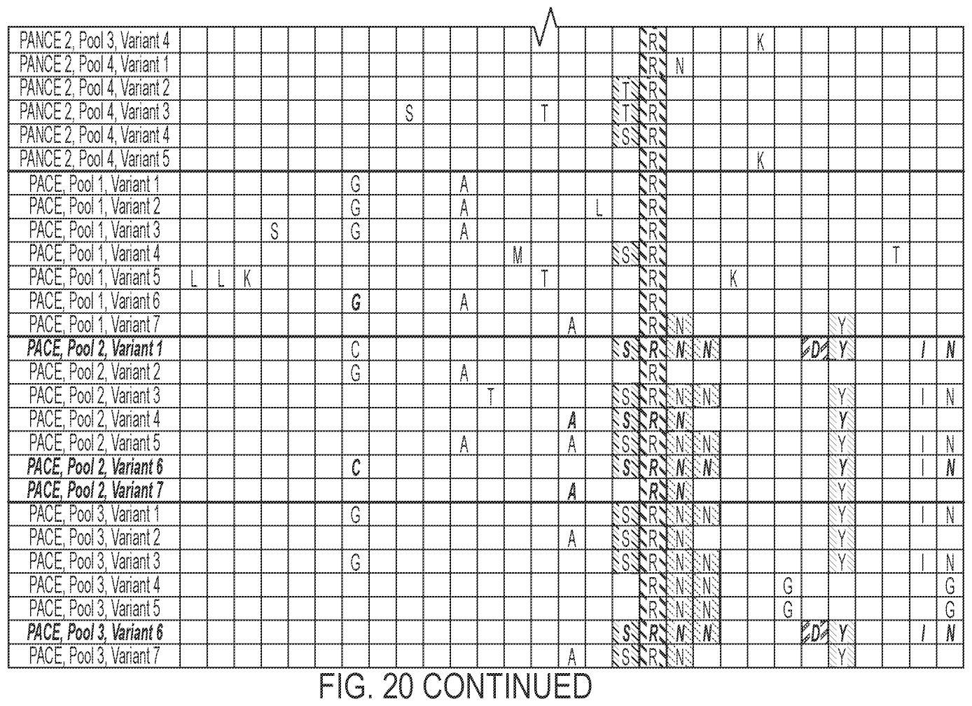

Analysis of several rounds of PACE on the adenosine deaminase TadA7.10 revealed enrichment of 11 additional consensus mutations across a variety of unique genotypes: R26G, V69A, V88A, A109S, T111R, D119N, H122N, Y147D, F149Y, T1661, and D167N (see FIGS. 2A-2B and FIG. 20). Accordingly, in some embodiments, the adenosine deaminase comprises several additional substitutions in TadA7.10 (SEQ ID NO: 315). In some embodiments, the adenosine deaminase comprises T111R, D119N, and F149Y substitutions in SEQ ID NO: 315, or a corresponding mutation in another adenosine deaminase. In particular embodiments, the adenosine deaminase comprises T111R, D119N, and F149Y substitutions, and further comprises at least one substitution selected from R26C, V88A, A109S, H122N, T1661, and D167N, in SEQ ID NO: 315, or a corresponding mutation in another adenosine deaminase.

Any of the adenosine deaminases described herein may be a truncated variant of any of the other adenosine deaminases described herein, e.g., any of the adenosine deaminases of SEQ ID NOs: 315-325, 433, 434, and 448. Exemplary truncated adenosine deaminases may comprise truncations of 1, 2, 3, 4, 5, 6, 7, 8, 9, 10, 11, 12, 13, 14, 15, or more than 15 amino acids from the N-terminus. Other exemplary truncated adenosine deaminases may comprise truncations of 1, 2, 3, 4, 5, 6, 7, 8, 9, 10, 11, 12, 13, 14, 15, or more than 15 amino acids from the C-terminus. In some embodiments, the adenosine deaminase domain comprises a trunacted version of the wild-type ecTadA, as set forth in SEQ ID NO: 324. Any of the adenosine deaminases described herein may include an N-terminal methionine (M) amino acid residue.

It should be appreciated that any of the mutations provided herein (e.g., based on the ecTadA amino acid sequence of SEQ ID NO: 315) may be introduced into other adenosine deaminases, such as S. aureus TadA (saTadA), or other adenosine deaminases (e.g., bacterial adenosine deaminases), such as those sequences provided below. It would be apparent to the skilled artisan how to identify amino acid residues from other adenosine deaminases that are homologous to the mutated residues in ecTadA. Thus, any of the mutations identified in ecTadA may be made in other adenosine deaminases that have homologous amino acid residues (see FIG. 28). Any of the mutations provided herein may be made individually or in any combination in ecTadA or another adenosine deaminase. Any of the mutated deaminases provided herein may be used in the context of adenine base editor.

In certain aspects, the disclosure provides adenine base editors with broadened target sequence compatibility. In general, native ecTadA deaminates the adenine in the sequence UAC (e.g., the target sequence) of the anticodon loop of tRNA^{Arg}. Without wishing to be bound by any particular theory, in order to expand the utility of ABEs comprising one or more ecTadA deaminases, such as any of the adenosine deaminases provided herein, the adenosine deaminase proteins were optimized to recognize a wide variety of target sequences within the protospacer sequence without compromising the editing efficiency of the adenosine nucleobase editor complex. In some embodiments, the target sequence is an A in the center of a 5′-NAN-3′ sequence, wherein N is T, C, G, or A. In some embodiments, the target sequence comprises 5′-TAC-3′. In some embodiments, the target sequence comprises 5′-GAA-3′.

In certain embodiments, the adenosine deaminase domain comprises an adenosine deaminase that has a sequence with at least 80%, at least 85%, at least 90%, at least 95%, at least 98%, at least 99%, or at least 99.5% sequence identity to SEQ ID NO: 433. In certain embodiments, the adenosine deaminase domain comprises an adenosine deaminase that has a sequence with at least 80%, at least 85%, at least 90%, at least 95%, at least 98%, at least 99%, or at least 99.5% sequence identity to SEQ ID NO: 433. In certain embodiments, the adenosine deaminase domain comprises an adenosine deaminase that has a sequence with at least 80%, at least 85%, at least 90%, at least 95%, at least 98%, at least 99%, or at least 99.5% sequence identity to SEQ ID NO: 434.

In some embodiments, any of the adenosine deaminase domains described herein may comprise an amino acid sequence having 1, 2, 3, 4, 5, 6, 7, 8, 9, 10, 11, 12, 13, 14, 15, 16, 17, 18, 19, 20, 25, 30, or more than 30 amino acids that differ relative to the amino acid sequence of either of SEQ ID NOs: 433 and 434. These differences may comprise amino acids that have been inserted, deleted, or substituted relative to the amino acid sequence of the wild type enzyme. In some embodiments, the disclosed adenosine deaminase domains contain stretches of about 50, about 75, about 100, about 125, about 150, about 175, about 200, about 300, about 400, about 500, or more than 500 consecutive amino acids in common with either of SEQ ID NOs: 433 and 434.

In some embodiments, any of the adenosine deaminase domains described herein may comprise an amino acid sequence having 1, 2, 3, 4, 5, 6, 7, 8, 9, 10, 11, 12, 13, 14, 15, 16, 17, 18, 19, 20, 25, 30, or more than 30 amino acids that differ relative to the amino acid sequence of a wild type adenosine deaminase (e.g., ecTadA). In some embodiments, any of the adenosine deaminase domains described herein may comprise an amino acid sequence having 1, 2, 3, 4, 5, 6, 7, 8, 9, 10, 11, 12, 13, 14, 15, 16, 17, 18, 19, 20, 25, 30, or more than 30 amino acids that differ relative to the amino acid sequence of any one of SEQ ID NOs: 315-325 and 448. These differences may comprise amino acids that have been inserted, deleted, or substituted relative to the amino acid sequence of the wild type enzyme. In some embodiments, the disclosed adenosine deaminase domains contain stretches of about 50, about 75, about 100, about 125, about 150, about 175, about 200, about 300, about 400, about 500, or more than 500 consecutive amino acids in common with a wild type adenosine deaminase. In some embodiments, the disclosed adenosine deaminase domains contain stretches of about 50, about 75, about 100, about 125, about 150, about 175, about 200, about 300, about 400, about 500, or more than 500 consecutive amino acids in common with any one of SEQ ID NOs: 315-325 and 448. In some embodiments, the adenosine deaminase domains comprise truncations at the N-terminus or C-terminus relative to a wild-type adenosine deaminase. In some embodiments, the adenosine deaminase domains comprise truncations at the N-terminus or C-terminus relative to any one of SEQ ID NOs: 315-325 and 448.

Exemplary adenine nucleobase editors include, but are not limited to, ABE7.10 (or ABEmax), ABE8e, SaKKH-ABE8e, NG-ABE8e, ABE-xCas9, SaKKH-ABE7.10, NG-ABE7.10, ABE7.10-VRQR, ABE8e-NRTH, ABE8e-NRRH, ABE8e-NRCH, NG-CP1041-ABE8e, ABE8e-CP1041, ABE8e-CP1028, and ABE8e-VRQR. In certain embodiments, the adenine nucleobase editor used in the disclosed methods is an ABE8e or an ABE7.10. ABE8e may be referred to herein as “ABE8” or “ABE8.0”. The ABE8e nucleobase editor and variants thereof may comprise an adenosine deaminase domain containing a TadA-8e adenosine deaminase monomer (monomer form) or a TadA-8e adenosine deaminase homodimer or heterodimer (dimer form). Other ABEs may be used to deaminate an A nucleobase in accordance with the disclosure.

Exemplary adenosine deaminase variants of the disclosure are described below. In certain embodiments, the adenosine deaminase domain comprises an adenosine deaminase that has a sequence with at least 80%, at least 85%, at least 90%, at least 95%, at least 98%, at least 99%, or at least 99.5% sequence identity to one of the following:

TadA7.10 (E. coli)
(SEQ ID NO: 315)
MSEVEFSHEYWMRHALTLAKRARDEREVPVGAVLVLNNRVIGEGWNRAIGLHDPTAHA
EIMALRQGGLVMQNYRLIDATLYVTFEPCVMCAGAMIHSRIGRVVFGVRNAKTGAAGS
LMDVLHYPGMNHRVEITEGILADECAALLCYFFRMPRQVFNAQKKAQSSTD
TadA7.10 (V106W)(E. coli)
(SEQ ID NO: 316)
MSEVEFSHEYWMRHALTLAKRARDEREVPVGAVLVLNNRVIGEGWNRAIGLHDPTAHA
EIMALRQGGLVMQNYRLIDATLYVTFEPCVMCAGAMIHSRIGRVVFGWRNAKTGAAGS
LMDVLHYPGMNHRVEITEGILADECAALLCYFFRMPRQVFNAQKKAQSSTD
TadA-8e (E. coli)
(SEQ ID NO: 433)
SEVEFSHEYWMRHALTLAKRARDEREVPVGAVLVLNNRVIGEGWNRAIGLHDPTAHAEI
MALRQGGLVMQNYRLIDATLYVTFEPCVMCAGAMIHSRIGRVVFGVRNSKRGAAGSLM
NVLNYPGMNHRVEITEGILADECAALLCDFYRMPRQVFNAQKKAQSSIN
TadA-8e(V106W)(E. coli)
(SEQ ID NO: 434)
SEVEFSHEYWMRHALTLAKRARDEREVPVGAVLVLNNRVIGEGWNRAIGLHDPTAHAEI
MALRQGGLVMQNYRLIDATLYVTFEPCVMCAGAMIHSRIGRVVFGWRNSKRGAAGSL
MNVLNYPGMNHRVEITEGILADECAALLCDFYRMPRQVFNAQKKAQSSIN
Staphylococcus aureus TadA:
(SEQ ID NO: 317)
MGSHMTNDIYFMTLAIEEAKKAAQLGEVPIGAIITKDDEVIARAHNLRETLQQPTAHAEH
IAIERAAKVLGSWRLEGCTLYVTLEPCVMCAGTIVMSRIPRVVYGADDPKGGCSGSLMN
LLQQSNFNHRAIVDKGVLKEACSTLLTTFFKNLRANKKSTN
Bacillus subtilis TadA:
(SEQ ID NO: 318)
MTQDELYMKEAIKEAKKAEEKGEVPIGAVLVINGEIIARAHNLRETEQRSIAHAEMLVID
EACKALGTWRLEGATLYVTLEPCPMCAGAVVLSRVEKVVFGAFDPKGGCSGTLMNLLQ
EERFNHQAEVVSGVLEEECGGMLSAFFRELRKKKKAARKNLSE
Salmonella typhimurium (S. typhimurium) TadA:
(SEQ ID NO: 319)
MPPAFITGVTSLSDVELDHEYWMRHALTLAKRAWDEREVPVGAVLVHNHRVIGEGWNR
PIGRHDPTAHAEIMALRQGGLVLQNYRLLDTTLYVTLEPCVMCAGAMVHSRIGRVVFGA
RDAKTGAAGSLIDVLHHPGMNHRVEIIEGVLRDECATLLSDFFRMRRQEIKALKKADRA
EGAGPAV
Shewanella putrefaciens (S. putrefaciens) TadA:
(SEQ ID NO: 320)
MDEYWMQVAMQMAEKAEAAGEVPVGAVLVKDGQQIATGYNLSISQHDPTAHAEILCL
RSAGKKLENYRLLDATLYITLEPCAMCAGAMVHSRIARVVYGARDEKTGAAGTVVNLL
QHPAFNHQVEVTSGVLAEACSAQLSRFFKRRRDEKKALKLAQRAQQGIE
Haemophilus influenzae F3031 (H. influenzae) TadA:
(SEQ ID NO: 321)
MDAAKVRSEFDEKMMRYALELADKAEALGEIPVGAVLVDDARNIIGEGWNLSIVQSDPT
AHAEIIALRNGAKNIQNYRLLNSTLYVTLEPCTMCAGAILHSRIKRLVFGASDYKTGAIGS
RFHFFDDYKMNHTLEITSGVLAEECSQKLSTFFQKRREEKKIEKALLKSLSDK
Caulobacter crescentus (C. crescentus) TadA:
(SEQ ID NO: 322)
MRTDESEDQDHRMMRLALDAARAAAEAGETPVGAVILDPSTGEVIATAGNGPIAAHDPT
AHAEIAAMRAAAAKLGNYRLTDLTLVVTLEPCAMCAGAISHARIGRVVFGADDPKGGA
VVHGPKFFAQPTCHWRPEVTGGVLADESADLLRGFFRARRKAKI
Geobacter sulfurreducens (G. sulfurreducens) TadA:
(SEQ ID NO: 323)
MSSLKKTPIRDDAYWMGKAIREAAKAAARDEVPIGAVIVRDGAVIGRGHNLREGSNDPS
AHAEMIAIRQAARRSANWRLTGATLYVTLEPCLMCMGAIILARLERVVFGCYDPKGAAG
SLYDLSADPRLNHQVRLSPGVCQEECGTMLSDFFRDLRRRKKAKATPALFIDERKVPPEP
Streptococcus pyogenes (S. pyogenes) TadA
(SEQ ID NO: 448)
MPYSLEEQTYFMQEALKEAEKSLQKAEIPIGCVIVKDGEIIGRGHNAREESNQAIMHAEI
MAINEANAHEGNWRLLDTTLFVTIEPCVMCSGAIGLARIPHVIYGASNQKFGGADSLYQI
LTDERLNHRVQVERGLLAADCANIMQTFFRQGRERKKIAKHLIKEQSDPFD

In some embodiments, the adenosine deaminase domain comprises an N-terminal truncated E. coli TadA. In certain embodiments, the adenosine deaminase comprises the amino acid sequence:

(SEQ ID NO: 324)
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD.

In some embodiments, the TadA deaminase is a full-length E. coli TadA deaminase (ecTadA). For example, in certain embodiments, the adenosine deaminase domain comprises a deaminase that comprises the amino acid sequence:

(SEQ ID NO: 325)
MRRAFITGVFFLSEVEFSHEYWMRHALTLAKRAWDEREVPVGAVLVHNNR
VIGEGWNRPIGRHDPTAHAEIMALRQGGLVMQNYRLIDATLYVTLEPCVM
CAGAMIHSRIGRVVFGARDAKTGAAGSLMDVLHHPGMNHRVEITEGILAD
ECAALLSDFFRMRRQEIKAQKKAQSSTD

Any two or more of the adenosine deaminases described herein may be connected to one another (e.g., by a linker, such as a peptide linker) within an adenosine deaminase domain of the base editors provided herein. In some embodiments, the base editor comprises two adenosine deaminases (e.g., a first adenosine deaminase and a second adenosine deaminase). For instance, in certain embodiments, the base editors provided herein may contain exactly two adenosine deaminases. In some embodiments, the first and second adenosine deaminases are any of the adenosine deaminases provided herein. In some embodiments, the adenosine deaminases are the same. In some embodiments, the adenosine deaminases are different. In some embodiments, the first adenosine deaminase and second adenosine deaminase are derived from the same bacterial species. In some embodiments, the first adenosine deaminase and second adenosine deaminase are derived from different bacterial species.

In some embodiments, the base editor comprises a heterodimer of a first adenosine deaminase and a second adenosine deaminase. In some embodiments, the first adenosine deaminase is N-terminal to the second adenosine deaminase in the base editor. In some embodiments, the first adenosine deaminase is C-terminal to the second adenosine deaminase in the base editor. In some embodiments, the first adenosine deaminase and the second deaminase are fused directly to each other or via a linker. In some embodiments, the first adenosine deaminase is fused N-terminal to the napDNAbp via a linker, and the second deaminase is fused C-terminal to the napDNAbp via a linker. In other embodiments, the second adenosine deaminase is fused N-terminal to the napDNAbp via a linker, and the first deaminase is fused C-terminal to the napDNAbp via a linker.

In some embodiments, the base editors disclosed herein comprise a heterodimer of a first adenosine deaminase and a second adenosine deaminase, wherein the first adenosine deaminase comprises a wild-type TadA sequence (e.g., the wild-type ecTadA sequence of SEQ ID NO: 325 or the truncated wild-type ecTadA sequence of SEQ ID NO: 324; and the second adenosine deaminase comprises any of the adenosine deaminase variants disclosed herein. In some embodiments, the base editors disclosed herein comprise a heterodimer of a first adenosine deaminase that comprises a sequence with at least 80%, 85%, 90%, 95%, 98%, 99%, or 99.5% sequence identity to SEQ ID NO: 324; and the second adenosine deaminase comprises a sequence with at least 80%, 85%, 90%, 95%, 98%, 99%, or 99.5% sequence identity to SEQ ID NO: 433 or 434.

In particular embodiments, the base editors disclosed herein comprise a heterodimer of a first adenosine deaminase that comprises a sequence with at least 90%, 95%, 98%, 99%, or 99.5% sequence identity to SEQ ID NO: 324; and the second adenosine deaminase comprises a sequence with at least 90%, 95%, 98%, 99%, or 99.5% sequence identity to SEQ ID NO: 433. In some embodiments, the base editors disclosed herein comprise a heterodimer of a first adenosine deaminase that comprises a sequence with at least 90%, 95%, 98%, 99%, or 99.5% sequence identity to SEQ ID NO: 324; and the second adenosine deaminase comprises a sequence with at least 90%, 95%, 98%, 99%, or 99.5% sequence identity to SEQ ID NO: 434.

The amino acid sequences of the adenosine deaminases provided herein include an initial methionine (M) residue, and every provided amino acid substitutions in these deaminases are described relative to that initial methionine residue. It should be appreciated that, in some embodiments, the adenosine deaminases provided herein may not contain an initial M residue, and accordingly the positions of the provided substitutions may shift by one.

napDNAbp Domains

The base editors described herein comprise a nucleic acid programmable DNA binding (napDNAbp) domain. The napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp domain to localize and bind to a complementary sequence of the target strand. Binding of the napDNAbp domain to a complementary sequence enables the nucleobase modification domain (i.e., the adenosine deaminase domain) of the base editor to access and enzymatically deaminate a target adenine base in the target strand.

The napDNAbp can be a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., Science 337:816-821(2012), the entire contents of which is hereby incorporated by reference.

Without wishing to be bound by any particular theory, the binding mechanism of a napDNAbp—guide RNA complex, in general, includes the step of forming an R-loop whereby the napDNAbp induces the unwinding of a double-strand DNA target, thereby separating the strands in the region bound by the napDNAbp. The guideRNA protospacer then hybridizes to the “target strand.” This displaces a “non-target strand” that is complementary to the target strand, which forms the single strand region of the R-loop. In some embodiments, the napDNAbp includes one or more nuclease activities, which cuts the DNA leaving various types of lesions (e.g., a nick in one strand of the DNA). For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand.

The below description of various napDNAbps which can be used in connection with the disclosed adenosine deaminases is not meant to be limiting in any way. The adenine base editors may comprise the canonical SpCas9, or any ortholog Cas9 protein, or any variant Cas9 protein—including any naturally occurring variant, mutant, or otherwise engineered version of Cas9—that is known or which can be made or evolved through a directed evolutionary or otherwise mutagenic process. In various embodiments, the napDNAbp has a nickase activity, i.e., only cleave one strand of the target DNA sequence. In other embodiments, the napDNAbp has an inactive nuclease, e.g., are “dead” proteins. Other variant Cas9 proteins that may be used are those having a smaller molecular weight than the canonical SpCas9 (e.g., for easier delivery) or having modified or rearranged primary amino acid sequence (e.g., the circular permutant forms). The adenine base editors described herein may also comprise Cas9 equivalents, including Cas12a/Cpf1 and Cas12b proteins. The napDNAbps used herein (e.g., SpCas9, SaCas9, or SaCas9 variant or SpCas9 variant) may also may also contain various modifications that alter/enhance their PAM specifities. The disclosure contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a reference SpCas9 canonical sequence (set forth in SEQ ID NO: 326), a reference SaCas9 canonical sequence (set forth in SEQ ID NO: 377) or a reference Cas9 equivalent (e.g., Cas12a/Cpf1).

In some embodiments, the napDNAbp directs cleavage of one or both strands at the location of a target sequence, such as within the target sequence and/or within the complement of the target sequence. In some embodiments, the napDNAbp directs cleavage of one or both strands within about 1, 2, 3, 4, 5, 6, 7, 8, 9, 10, 15, 20, 25, 50, 100, 200, 500, or more base pairs from the first or last nucleotide of a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A in reference to the canonical SpCas9 sequence, or to equivalent amino acid positions in other Cas9 variants or Cas9 equivalents.

In some embodiments, the napDNAbp domain may comprise more than one napDNAbp proteins. Accordingly, in some embodiments, any of the disclosed base editors may contain a first napDNAbp domain and a second napDNAbp domain. In some embodiments, the napDNAbp domain (or the first and second napDNAbp domain, respectively) comprises a first Cas homolog or variant and a second Cas homolog or variant (e.g., a first Cas variant comprising a Cas9-NG and a second Cas variant comprising a Cas9-CP1041, e.g., “SpCas9-NG-CP1041”). In some embodiments, the first Cas variant comprises a Cas9-NG, and the second Cas variant comprises a SpCas9-VRQR.

As used herein, the term “Cas protein” refers to a full-length Cas protein obtained from nature, a recombinant Cas protein having a sequences that differs from a naturally occurring Cas protein, or any fragment of a Cas protein that nevertheless retains all or a significant amount of the requisite basic functions needed for the disclosed methods, i.e., (i) possession of nucleic-acid programmable binding of the Cas protein to a target DNA, and (ii) ability to nick the target DNA sequence on one strand. The Cas proteins contemplated herein embrace CRISPR Cas9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference.

The term “Cas9” or “Cas9 domain” embraces any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular napDNAbp that is employed in the adenine base editors of the disclosure.

Additional Cas9 sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference), and also provided below.

Examples of Cas9 and Cas9 equivalents are provided as follows; however, these specific examples are not meant to be limiting. The base editors of the present disclosure may use any suitable napDNAbp, including any suitable Cas9 or Cas9 equivalent.

A. Wild Type Canonical SpCas9

In one embodiment, the base editor constructs described herein may comprise the “canonical SpCas9” nuclease from S. pyogenes, which has been widely used as a tool for genome engineering. This Cas9 protein is a large, multi-domain protein containing two distinct nuclease domains. Point mutations can be introduced into Cas9 to abolish one or both nuclease activities, resulting in a nickase Cas9 (nCas9) or dead Cas9 (dCas9), respectively, that still retains its ability to bind DNA in a sgRNA-programmed manner. In principle, when fused to another protein or domain, Cas9 or variant thereof (e.g., nCas9) can target that protein to virtually any DNA sequence simply by co-expression with an appropriate sgRNA. As used herein, the canonical SpCas9 protein refers to the wild type protein from Streptococcus pyogenes having the following amino acid sequence:

| Description | Sequence | SEQ ID NO: |
|---|---|---|
| SpCas 9 | MDKKYSIGLDIGTNSVGWAVITDEYKVPSKKFKVLGNTDRHSIKKNLIGALLFDS | SEQ ID NO: |
| Streptococcus | GETAEATRLKRTARRRYTRRKNRICYLQEIFSNEMAKVDDSFFHRLEESFLVEED | 326 |
| pyogenes | KKHERHPIFGNIVDEVAYHEKYPTIYHLRKKLVDSTDKADLRLIYLALAHMIKFR | |
| M1 | GHFLIEGDLNPDNSDVDKLFIQLVQTYNQLFEENPINASGVDAKAILSARLSKSR | |
| SwissProt | RLENLIAQLPGEKKNGLFGNLIALSLGLTPNFKSNFDLAEDAKLQLSKDTYDDDL | |
| Accession | DNLLAQIGDQYADLFLAAKNLSDAILLSDILRVNTEITKAPLSASMIKRYDEHHQ | |
| No. Q99ZW2 | DLTLLKALVRQQLPEKYKEIFFDQSKNGYAGYIDGGASQEEFYKFIKPILEKMDG | |
| Wild type | TEELLVKLNREDLLRKQRTFDNGSIPHQIHLGELHAILRRQEDFYPFLKDNREKI | |
| | EKILTFRIPYYVGPLARGNSRFAWMTRKSEETITPWNFEEVVDKGASAQSFIERM | |
| | TNFDKNLPNEKVLPKHSLLYEYFTVYNELTKVKYVTEGMRKPAFLSGEQKKAIVD | |
| | LLFKTNRKVTVKQLKEDYFKKIECFDSVEISGVEDRFNASLGTYHDLLKIIKDKD | |
| | FLDNEENEDILEDIVLTLTLFEDREMIEERLKTYAHLFDDKVMKQLKRRRYTGWG | |
| | RLSRKLINGIRDKQSGKTILDFLKSDGFANRNFMQLIHDDSLTFKEDIQKAQVSG | |
| | QGDSLHEHIANLAGSPAIKKGILQTVKVVDELVKVMGRHKPENIVIEMARENQTT | |
| | QKGQKNSRERMKRIEEGIKELGSQILKEHPVENTQLQNEKLYLYYLQNGRDMYVD | |
| | QELDINRLSDYDVDHIVPQSFLKDDSIDNKVLTRSDKNRGKSDNVPSEEVVKKMK | |
| | NYWRQLLNAKLITQRKFDNLTKAERGGLSELDKAGFIKRQLVETRQITKHVAQIL | |
| | DSRMNTKYDENDKLIREVKVITLKSKLVSDFRKDFQFYKVREINNYHHAHDAYLN | |
| | AVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVEKGKSK | |
| | KLKSVKELLGITIMERSSFEKNPIDFLEAKGYKEVKKDLIIKLPKYSLFELENGR | |
| | KRMLASAGELQKGNELALPSKYVNFLYLASHYEKLKGSPEDNEQKQLFVEQHKHY | |
| | LDEIIEQISEFSKRVILADANLDKVLSAYNKHRDKPIREQAENIIHLFTLTNLGA | |
| | PAAFKYFDTTIDRKRYTSTKEVLDATLIHQSITGLYETRIDLSQLGGD | |
| SpCas 9 | ATGGATAAAAAATATAGCATTGGCCTGGATATTGGCACCAACAGCGTGGGCTGGG | SEQ ID NO: |
| Reverse | CGGTGATTACCGATGAATATAAAGTGCCGAGCAAAAAATTTAAAGTGCTGGGCAA | 327 |
| translation | CACCGATCGCCATAGCATTAAAAAAAACCTGATTGGCGCGCTGCTGTTTGATAGC | |
| of | GGCGAAACCGCGGAAGCGACCCGCCTGAAACGCACCGCGCGCCGCCGCTATACCC | |
| SwissProt | GCCGCAAAAACCGCATTTGCTATCTGCAGGAAATTTTTAGCAACGAAATGGCGAA | |
| Accession | AGTGGATGATAGCTTTTTTCATCGCCTGGAAGAAAGCTTTCTGGTGGAAGAAGAT | |
| No. Q99ZW2 | AAAAAACATGAACGCCATCCGATTTTTGGCAACATTGTGGATGAAGTGGCGTATC | |
| Streptococcus | ATGAAAAATATCCGACCATTTATCATCTGCGCAAAAAACTGGTGGATAGCACCGA | |
| pyogenes | TAAAGCGGATCTGCGCCTGATTTATCTGGCGCTGGCGCATATGATTAAATTTCGC | |
| | GGCCATTTTCTGATTGAAGGCGATCTGAACCCGGATAACAGCGATGTGGATAAAC | |
| | TGTTTATTCAGCTGGTGCAGACCTATAACCAGCTGTTTGAAGAAAACCCGATTAA | |
| | CGCGAGCGGCGTGGATGCGAAAGCGATTCTGAGCGCGCGCCTGAGCAAAAGCCGC | |
| | CGCCTGGAAAACCTGATTGCGCAGCTGCCGGGCGAAAAAAAAAACGGCCTGTTTG | |
| | GCAACCTGATTGCGCTGAGCCTGGGCCTGACCCCGAACTTTAAAAGCAACTTTGA | |
| | TCTGGCGGAAGATGCGAAACTGCAGCTGAGCAAAGATACCTATGATGATGATCTG | |
| | GATAACCTGCTGGCGCAGATTGGCGATCAGTATGCGGATCTGTTTCTGGCGGCGA | |
| | AAAACCTGAGCGATGCGATTCTGCTGAGCGATATTCTGCGCGTGAACACCGAAAT | |
| | TACCAAAGCGCCGCTGAGCGCGAGCATGATTAAACGCTATGATGAACATCATCAG | |
| | GATCTGACCCTGCTGAAAGCGCTGGTGCGCCAGCAGCTGCCGGAAAAATATAAAG | |
| | AAATTTTTTTTGATCAGAGCAAAAACGGCTATGCGGGCTATATTGATGGCGGCGC | |
| | GAGCCAGGAAGAATTTTATAAATTTATTAAACCGATTCTGGAAAAAATGGATGGC | |
| | ACCGAAGAACTGCTGGTGAAACTGAACCGCGAAGATCTGCTGCGCAAACAGCGCA | |
| | CCTTTGATAACGGCAGCATTCCGCATCAGATTCATCTGGGCGAACTGCATGCGAT | |
| | TCTGCGCCGCCAGGAAGATTTTTATCCGTTTCTGAAAGATAACCGCGAAAAAATT | |
| | GAAAAAATTCTGACCTTTCGCATTCCGTATTATGTGGGCCCGCTGGCGCGCGGCA | |
| | ACAGCCGCTTTGCGTGGATGACCCGCAAAAGCGAAGAAACCATTACCCCGTGGAA | |
| | CTTTGAAGAAGTGGTGGATAAAGGCGCGAGCGCGCAGAGCTTTATTGAACGCATG | |
| | ACCAACTTTGATAAAAACCTGCCGAACGAAAAAGTGCTGCCGAAACATAGCCTGC | |
| | TGTATGAATATTTTACCGTGTATAACGAACTGACCAAAGTGAAATATGTGACCGA | |
| | AGGCATGCGCAAACCGGCGTTTCTGAGCGGCGAACAGAAAAAAGCGATTGTGGAT | |
| | CTGCTGTTTAAAACCAACCGCAAAGTGACCGTGAAACAGCTGAAAGAAGATTATT | |
| | TTAAAAAAATTGAATGCTTTGATAGCGTGGAAATTAGCGGCGTGGAAGATCGCTT | |
| | TAACGCGAGCCTGGGCACCTATCATGATCTGCTGAAAATTATTAAAGATAAAGAT | |
| | TTTCTGGATAACGAAGAAAACGAAGATATTCTGGAAGATATTGTGCTGACCCTGA | |
| | CCCTGTTTGAAGATCGCGAAATGATTGAAGAACGCCTGAAAACCTATGCGCATCT | |
| | GTTTGATGATAAAGTGATGAAACAGCTGAAACGCCGCCGCTATACCGGCTGGGGC | |
| | CGCCTGAGCCGCAAACTGATTAACGGCATTCGCGATAAACAGAGCGGCAAAACCA | |
| | TTCTGGATTTTCTGAAAAGCGATGGCTTTGCGAACCGCAACTTTATGCAGCTGAT | |
| | TCATGATGATAGCCTGACCTTTAAAGAAGATATTCAGAAAGCGCAGGTGAGCGGC | |
| | CAGGGCGATAGCCTGCATGAACATATTGCGAACCTGGCGGGCAGCCCGGCGATTA | |
| | AAAAAGGCATTCTGCAGACCGTGAAAGTGGTGGATGAACTGGTGAAAGTGATGGG | |
| | CCGCCATAAACCGGAAAACATTGTGATTGAAATGGCGCGCGAAAACCAGACCACC | |
| | CAGAAAGGCCAGAAAAACAGCCGCGAACGCATGAAACGCATTGAAGAAGGCATTA | |
| | AAGAACTGGGCAGCCAGATTCTGAAAGAACATCCGGTGGAAAACACCCAGCTGCA | |
| | GAACGAAAAACTGTATCTGTATTATCTGCAGAACGGCCGCGATATGTATGTGGAT | |
| | CAGGAACTGGATATTAACCGCCTGAGCGATTATGATGTGGATCATATTGTGCCGC | |
| | AGAGCTTTCTGAAAGATGATAGCATTGATAACAAAGTGCTGACCCGCAGCGATAA | |
| | AAACCGCGGCAAAAGCGATAACGTGCCGAGCGAAGAAGTGGTGAAAAAAATGAAA | |
| | AACTATTGGCGCCAGCTGCTGAACGCGAAACTGATTACCCAGCGCAAATTTGATA | |
| | ACCTGACCAAAGCGGAACGCGGCGGCCTGAGCGAACTGGATAAAGCGGGCTTTAT | |
| | TAAACGCCAGCTGGTGGAAACCCGCCAGATTACCAAACATGTGGCGCAGATTCTG | |
| | GATAGCCGCATGAACACCAAATATGATGAAAACGATAAACTGATTCGCGAAGTGA | |
| | AAGTGATTACCCTGAAAAGCAAACTGGTGAGCGATTTTCGCAAAGATTTTCAGTT | |
| | TTATAAAGTGCGCGAAATTAACAACTATCATCATGCGCATGATGCGTATCTGAAC | |
| | GCGGTGGTGGGCACCGCGCTGATTAAAAAATATCCGAAACTGGAAAGCGAATTTG | |
| | TGTATGGCGATTATAAAGTGTATGATGTGCGCAAAATGATTGCGAAAAGCGAACA | |
| | GGAAATTGGCAAAGCGACCGCGAAATATTTTTTTTATAGCAACATTATGAACTTT | |
| | TTTAAAACCGAAATTACCCTGGCGAACGGCGAAATTCGCAAACGCCCGCTGATTG | |
| | AAACCAACGGCGAAACCGGCGAAATTGTGTGGGATAAAGGCCGCGATTTTGCGAC | |
| | CGTGCGCAAAGTGCTGAGCATGCCGCAGGTGAACATTGTGAAAAAAACCGAAGTG | |
| | CAGACCGGCGGCTTTAGCAAAGAAAGCATTCTGCCGAAACGCAACAGCGATAAAC | |
| | TGATTGCGCGCAAAAAAGATTGGGATCCGAAAAAATATGGCGGCTTTGATAGCCC | |
| | GACCGTGGCGTATAGCGTGCTGGTGGTGGCGAAAGTGGAAAAAGGCAAAAGCAAA | |
| | AAACTGAAAAGCGTGAAAGAACTGCTGGGCATTACCATTATGGAACGCAGCAGCT | |
| | TTGAAAAAAACCCGATTGATTTTCTGGAAGCGAAAGGCTATAAAGAAGTGAAAAA | |
| | AGATCTGATTATTAAACTGCCGAAATATAGCCTGTTTGAACTGGAAAACGGCCGC | |
| | AAACGCATGCTGGCGAGCGCGGGCGAACTGCAGAAAGGCAACGAACTGGCGCTGC | |
| | CGAGCAAATATGTGAACTTTCTGTATCTGGCGAGCCATTATGAAAAACTGAAAGG | |
| | CAGCCCGGAAGATAACGAACAGAAACAGCTGTTTGTGGAACAGCATAAACATTAT | |
| | CTGGATGAAATTATTGAACAGATTAGCGAATTTAGCAAACGCGTGATTCTGGCGG | |
| | ATGCGAACCTGGATAAAGTGCTGAGCGCGTATAACAAACATCGCGATAAACCGAT | |
| | TCGCGAACAGGCGGAAAACATTATTCATCTGTTTACCCTGACCAACCTGGGCGCG | |
| | CCGGCGGCGTTTAAATATTTTGATACCACCATTGATCGCAAACGCTATACCAGCA | |
| | CCAAAGAAGTGCTGGATGCGACCCTGATTCATCAGAGCATTACCGGCCTGTATGA | |
| | AACCCGCATTGATCTGAGCCAGCTGGGCGGCGAT | |

The base editors described herein may include canonical SpCas9, or any variant thereof having at least 80%, at least 85%, at least 90%, at least 95%, or at least 99% sequence identity with a wild type Cas9 sequence provided above. These variants may include SpCas9 variants containing one or more mutations, including any known mutation reported with the SwissProt Accession No. Q99ZW2 entry, which include:

| SpCas9 mutation (relative to | Function/Characteristic (as reported) |
|---|---|
| the amino acid sequence | (see UniProtKB - Q99ZW2 |
| of the canonical SpCas9 | (CAS9_STRPT1) entry - |
| sequence, SEQ ID NO: 5) | incorporated herein by reference) |
| D10A | Nickase mutant which cleaves the |
| | protospacer strand (but no cleavage of |
| | non-protospacer strand) |
| S15A | Decreased DNA cleavage activity |
| R66A | Decreased DNA cleavage activity |
| R70A | No DNA cleavage |
| R74A | Decreased DNA cleavage |
| R78A | Decreased DNA cleavage |
| 97-150 deletion | No nuclease activity |
| R165A | Decreased DNA cleavage |
| 175-307 deletion | About 50% decreased DNA cleavage |
| 312-409 deletion | No nuclease activity |
| E762A | Nickase |
| H840A | Nickase mutant which cleaves the non- |
| | protospacer strand but does not cleave |
| | the protospacer strand |
| N854A | Nickase |
| N863A | Nickase |
| H982A | Decreased DNA cleavage |
| D986A | Nickase |
| 1099-1368 deletion | No nuclease activity |
| R1333A | Reduced DNA binding |

Other wild type SpCas9 sequences that may be used in the present disclosure, include:

| Description | Sequence | SEQ ID NO: |
|---|---|---|
| SpCas9 | ATGGATAAGAAATACTCAATAGGCTTAGATATCGGCACAAATAGCGTCGGATGGGCGGT | SEQ ID NO: |
| Streptococcus | GATCACTGATGATTATAAGGTTCCGTCTAAAAAGTTCAAGGTTCTGGGAAATACAGACC | 328 |
| pyogenes | GCCACAGTATCAAAAAAAATCTTATAGGGGCTCTTTTATTTGGCAGTGGAGAGACAGCG | |
| MGAS1882 wild | GAAGCGACTCGTCTCAAACGGACAGCTCGTAGAAGGTATACACGTCGGAAGAATCGTAT | |
| type | TTGTTATCTACAGGAGATTTTTTCAAATGAGATGGCGAAAGTAGATGATAGTTTCTTTC | |
| NC_017053.1 | ATCGACTTGAAGAGTCTTTTTTGGTGGAAGAAGACAAGAAGCATGAACGTCATCCTATT | |
| | TTTGGAAATATAGTAGATGAAGTTGCTTATCATGAGAAATATCCAACTATCTATCATCT | |
| | GCGAAAAAAATTGGCAGATTCTACTGATAAAGCGGATTTGCGCTTAATCTATTTGGCCT | |
| | TAGCGCATATGATTAAGTTTCGTGGTCATTTTTTGATTGAGGGAGATTTAAATCCTGAT | |
| | AATAGTGATGTGGACAAACTATTTATCCAGTTGGTACAAATCTACAATCAATTATTTGA | |
| | AGAAAACCCTATTAACGCAAGTAGAGTAGATGCTAAAGCGATTCTTTCTGCACGATTGA | |
| | GTAAATCAAGACGATTAGAAAATCTCATTGCTCAGCTCCCCGGTGAGAAGAGAAATGGC | |
| | TTGTTTGGGAATCTCATTGCTTTGTCATTGGGATTGACCCCTAATTTTAAATCAAATTT | |
| | TGATTTGGCAGAAGATGCTAAATTACAGCTTTCAAAAGATACTTACGATGATGATTTAG | |
| | ATAATTTATTGGCGCAAATTGGAGATCAATATGCTGATTTGTTTTTGGCAGCTAAGAAT | |
| | TTATCAGATGCTATTTTACTTTCAGATATCCTAAGAGTAAATAGTGAAATAACTAAGGC | |
| | TCCCCTATCAGCTTCAATGATTAAGCGCTACGATGAACATCATCAAGACTTGACTCTTT | |
| | TAAAAGCTTTAGTTCGACAACAACTTCCAGAAAAGTATAAAGAAATCTTTTTTGATCAA | |
| | TCAAAAAACGGATATGCAGGTTATATTGATGGGGGAGCTAGCCAAGAAGAATTTTATAA | |
| | ATTTATCAAACCAATTTTAGAAAAAATGGATGGTACTGAGGAATTATTGGTGAAACTAA | |
| | ATCGTGAAGATTTGCTGCGCAAGCAACGGACCTTTGACAACGGCTCTATTCCCCATCAA | |
| | ATTCACTTGGGTGAGCTGCATGCTATTTTGAGAAGACAAGAAGACTTTTATCCATTTTT | |
| | AAAAGACAATCGTGAGAAGATTGAAAAAATCTTGACTTTTCGAATTCCTTATTATGTTG | |
| | GTCCATTGGCGCGTGGCAATAGTCGTTTTGCATGGATGACTCGGAAGTCTGAAGAAACA | |
| | ATTACCCCATGGAATTTTGAAGAAGTTGTCGATAAAGGTGCTTCAGCTCAATCATTTAT | |
| | TGAACGCATGACAAACTTTGATAAAAATCTTCCAAATGAAAAAGTACTACCAAAACATA | |
| | GTTTGCTTTATGAGTATTTTACGGTTTATAACGAATTGACAAAGGTCAAATATGTTACT | |
| | GAGGGAATGCGAAAACCAGCATTTCTTTCAGGTGAACAGAAGAAAGCCATTGTTGATTT | |
| | ACTCTTCAAAACAAATCGAAAAGTAACCGTTAAGCAATTAAAAGAAGATTATTTCAAAA | |
| | AAATAGAATGTTTTGATAGTGTTGAAATTTCAGGAGTTGAAGATAGATTTAATGCTTCA | |
| | TTAGGCGCCTACCATGATTTGCTAAAAATTATTAAAGATAAAGATTTTTTGGATAATGA | |
| | AGAAAATGAAGATATCTTAGAGGATATTGTTTTAACATTGACCTTATTTGAAGATAGGG | |
| | GGATGATTGAGGAAAGACTTAAAACATATGCTCACCTCTTTGATGATAAGGTGATGAAA | |
| | CAGCTTAAACGTCGCCGTTATACTGGTTGGGGACGTTTGTCTCGAAAATTGATTAATGG | |
| | TATTAGGGATAAGCAATCTGGCAAAACAATATTAGATTTTTTGAAATCAGATGGTTTTG | |
| | CCAATCGCAATTTTATGCAGCTGATCCATGATGATAGTTTGACATTTAAAGAAGATATT | |
| | CAAAAAGCACAGGTGTCTGGACAAGGCCATAGTTTACATGAACAGATTGCTAACTTAGC | |
| | TGGCAGTCCTGCTATTAAAAAAGGTATTTTACAGACTGTAAAAATTGTTGATGAACTGG | |
| | TCAAAGTAATGGGGCATAAGCCAGAAAATATCGTTATTGAAATGGCACGTGAAAATCAG | |
| | ACAACTCAAAAGGGCCAGAAAAATTCGCGAGAGCGTATGAAACGAATCGAAGAAGGTAT | |
| | CAAAGAATTAGGAAGTCAGATTCTTAAAGAGCATCCTGTTGAAAATACTCAATTGCAAA | |
| | ATGAAAAGCTCTATCTCTATTATCTACAAAATGGAAGAGACATGTATGTGGACCAAGAA | |
| | TTAGATATTAATCGTTTAAGTGATTATGATGTCGATCACATTGTTCCACAAAGTTTCAT | |
| | TAAAGACGATTCAATAGACAATAAGGTACTAACGCGTTCTGATAAAAATCGTGGTAAAT | |
| | CGGATAACGTTCCAAGTGAAGAAGTAGTCAAAAAGATGAAAAACTATTGGAGACAACTT | |
| | CTAAACGCCAAGTTAATCACTCAACGTAAGTTTGATAATTTAACGAAAGCTGAACGTGG | |
| | AGGTTTGAGTGAACTTGATAAAGCTGGTTTTATCAAACGCCAATTGGTTGAAACTCGCC | |
| | AAATCACTAAGCATGTGGCACAAATTTTGGATAGTCGCATGAATACTAAATACGATGAA | |
| | AATGATAAACTTATTCGAGAGGTTAAAGTGATTACCTTAAAATCTAAATTAGTTTCTGA | |
| | CTTCCGAAAAGATTTCCAATTCTATAAAGTACGTGAGATTAACAATTACCATCATGCCC | |
| | ATGATGCGTATCTAAATGCCGTCGTTGGAACTGCTTTGATTAAGAAATATCCAAAACTT | |
| | GAATCGGAGTTTGTCTATGGTGATTATAAAGTTTATGATGTTCGTAAAATGATTGCTAA | |
| | GTCTGAGCAAGAAATAGGCAAAGCAACCGCAAAATATTTCTTTTACTCTAATATCATGA | |
| | ACTTCTTCAAAACAGAAATTACACTTGCAAATGGAGAGATTCGCAAACGCCCTCTAATC | |
| | GAAACTAATGGGGAAACTGGAGAAATTGTCTGGGATAAAGGGCGAGATTTTGCCACAGT | |
| | GCGCAAAGTATTGTCCATGCCCCAAGTCAATATTGTCAAGAAAACAGAAGTACAGACAG | |
| | GCGGATTCTCCAAGGAGTCAATTTTACCAAAAAGAAATTCGGACAAGCTTATTGCTCGT | |
| | AAAAAAGACTGGGATCCAAAAAAATATGGTGGTTTTGATAGTCCAACGGTAGCTTATTC | |
| | AGTCCTAGTGGTTGCTAAGGTGGAAAAAGGGAAATCGAAGAAGTTAAAATCCGTTAAAG | |
| | AGTTACTAGGGATCACAATTATGGAAAGAAGTTCCTTTGAAAAAAATCCGATTGACTTT | |
| | TTAGAAGCTAAAGGATATAAGGAAGTTAAAAAAGACTTAATCATTAAACTACCTAAATA | |
| | TAGTCTTTTTGAGTTAGAAAACGGTCGTAAACGGATGCTGGCTAGTGCCGGAGAATTAC | |
| | AAAAAGGAAATGAGCTGGCTCTGCCAAGCAAATATGTGAATTTTTTATATTTAGCTAGT | |
| | CATTATGAAAAGTTGAAGGGTAGTCCAGAAGATAACGAACAAAAACAATTGTTTGTGGA | |
| | GCAGCATAAGCATTATTTAGATGAGATTATTGAGCAAATCAGTGAATTTTCTAAGCGTG | |
| | TTATTTTAGCAGATGCCAATTTAGATAAAGTTCTTAGTGCATATAACAAACATAGAGAC | |
| | AAACCAATACGTGAACAAGCAGAAAATATTATTCATTTATTTACGTTGACGAATCTTGG | |
| | AGCTCCCGCTGCTTTTAAATATTTTGATACAACAATTGATCGTAAACGATATACGTCTA | |
| | CAAAAGAAGTTTTAGATGCCACTCTTATCCATCAATCCATCACTGGTCTTTATGAAACA | |
| | CGCATTGATTTGAGTCAGCTAGGAGGTGACTGA | |
| SpCas9 | MDKKYSIGLDIGTNSVGWAVITDDYKVPSKKFKVLGNTDRHSIKKNLIGALLFGSGETA | SEQ ID NO: |
| Streptococcus | EATRLKRTARRRYTRRKNRICYLQEIFSNEMAKVDDSFFHRLEESFLVEEDKKHERHPI | 329 |
| pyogenes | FGNIVDEVAYHEKYPTIYHLRKKLADSTDKADLRLIYLALAHMIKFRGHFLIEGDLNPD | |
| MGAS1882 wild | NSDVDKLFIQLVQIYNQLFEENPINASRVDAKAILSARLSKSRRLENLIAQLPGEKRNG | |
| type | LFGNLIALSLGLTPNFKSNFDLAEDAKLQLSKDTYDDDLDNLLAQIGDQYADLFLAAKN | |
| NC_017053.1 | LSDAILLSDILRVNSEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGAYHDLLKIIKDKDFLDNEENEDILEDIVLTLTLFEDRGMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGHSLHEQIANLAGSPAIKKGILQTVKIVDELVKVMGHKPENIVIEMARENQ | |
| | TTQKGQKNSRERMKRIEEGIKELGSQILKEHPVENTQLQNEKLYLYYLQNGRDMYVDQE | |
| | LDINRLSDYDVDHIVPQSFIKDDSIDNKVLTRSDKNRGKSDNVPSEEVVKKMKNYWRQL | |
| | LNAKLITQRKFDNLTKAERGGLSELDKAGFIKRQLVETRQITKHVAQILDSRMNTKYDE | |
| | NDKLIREVKVITLKSKLVSDFRKDFQFYKVREINNYHHAHDAYLNAVVGTALIKKYPKL | |
| | ESEFVYGDYKVYDVRKMIAKSEQEIGKATAKYFFYSNIMNFFKTEITLANGEIRKRPLI | |
| | ETNGETGEIVWDKGRDFATVRKVLSMPQVNIVKKTEVQTGGFSKESILPKRNSDKLIAR | |
| | KKDWDPKKYGGFDSPTVAYSVLVVAKVEKGKSKKLKSVKELLGITIMERSSFEKNPIDF | |
| | LEAKGYKEVKKDLIIKLPKYSLFELENGRKRMLASAGELQKGNELALPSKYVNFLYLAS | |
| | HYEKLKGSPEDNEQKQLFVEQHKHYLDEIIEQISEFSKRVILADANLDKVLSAYNKHRD | |
| | KPIREQAENIIHLFTLTNLGAPAAFKYFDTTIDRKRYTSTKEVLDATLIHQSITGLYET | |
| | RIDLSQLGGD | |
| SpCas9 | ATGGATAAAAAGTATTCTATTGGTTTAGACATCGGCACTAATTCCGTTGGATGGGCTGT | SEQ ID NO: |
| Streptococcus | CATAACCGATGAATACAAAGTACCTTCAAAGAAATTTAAGGTGTTGGGGAACACAGACC | 330 |
| pyogenes wild | GTCATTCGATTAAAAAGAATCTTATCGGTGCCCTCCTATTCGATAGTGGCGAAACGGCA | |
| type | GAGGCGACTCGCCTGAAACGAACCGCTCGGAGAAGGTATACACGTCGCAAGAACCGAAT | |
| SWBC2D7W014 | ATGTTACTTACAAGAAATTTTTAGCAATGAGATGGCCAAAGTTGACGATTCTTTCTTTC | |
| | ACCGTTTGGAAGAGTCCTTCCTTGTCGAAGAGGACAAGAAACATGAACGGCACCCCATC | |
| | TTTGGAAACATAGTAGATGAGGTGGCATATCATGAAAAGTACCCAACGATTTATCACCT | |
| | CAGAAAAAAGCTAGTTGACTCAACTGATAAAGCGGACCTGAGGTTAATCTACTTGGCTC | |
| | TTGCCCATATGATAAAGTTCCGTGGGCACTTTCTCATTGAGGGTGATCTAAATCCGGAC | |
| | AACTCGGATGTCGACAAACTGTTCATCCAGTTAGTACAAACCTATAATCAGTTGTTTGA | |
| | AGAGAACCCTATAAATGCAAGTGGCGTGGATGCGAAGGCTATTCTTAGCGCCCGCCTCT | |
| | CTAAATCCCGACGGCTAGAAAACCTGATCGCACAATTACCCGGAGAGAAGAAAAATGGG | |
| | TTGTTCGGTAACCTTATAGCGCTCTCACTAGGCCTGACACCAAATTTTAAGTCGAACTT | |
| | CGACTTAGCTGAAGATGCCAAATTGCAGCTTAGTAAGGACACGTACGATGACGATCTCG | |
| | ACAATCTACTGGCACAAATTGGAGATCAGTATGCGGACTTATTTTTGGCTGCCAAAAAC | |
| | CTTAGCGATGCAATCCTCCTATCTGACATACTGAGAGTTAATACTGAGATTACCAAGGC | |
| | GCCGTTATCCGCTTCAATGATCAAAAGGTACGATGAACATCACCAAGACTTGACACTTC | |
| | TCAAGGCCCTAGTCCGTCAGCAACTGCCTGAGAAATATAAGGAAATATTCTTTGATCAG | |
| | TCGAAAAACGGGTACGCAGGTTATATTGACGGCGGAGCGAGTCAAGAGGAATTCTACAA | |
| | GTTTATCAAACCCATATTAGAGAAGATGGATGGGACGGAAGAGTTGCTTGTAAAACTCA | |
| | ATCGCGAAGATCTACTGCGAAAGCAGCGGACTTTCGACAACGGTAGCATTCCACATCAA | |
| | ATCCACTTAGGCGAATTGCATGCTATACTTAGAAGGCAGGAGGATTTTTATCCGTTCCT | |
| | CAAAGACAATCGTGAAAAGATTGAGAAAATCCTAACCTTTCGCATACCTTACTATGTGG | |
| | GACCCCTGGCCCGAGGGAACTCTCGGTTCGCATGGATGACAAGAAAGTCCGAAGAAACG | |
| | ATTACTCCATGGAATTTTGAGGAAGTTGTCGATAAAGGTGCGTCAGCTCAATCGTTCAT | |
| | CGAGAGGATGACCAACTTTGACAAGAATTTACCGAACGAAAAAGTATTGCCTAAGCACA | |
| | GTTTACTTTACGAGTATTTCACAGTGTACAATGAACTCACGAAAGTTAAGTATGTCACT | |
| | GAGGGCATGCGTAAACCCGCCTTTCTAAGCGGAGAACAGAAGAAAGCAATAGTAGATCT | |
| | GTTATTCAAGACCAACCGCAAAGTGACAGTTAAGCAATTGAAAGAGGACTACTTTAAGA | |
| | AAATTGAATGCTTCGATTCTGTCGAGATCTCCGGGGTAGAAGATCGATTTAATGCGTCA | |
| | CTTGGTACGTATCATGACCTCCTAAAGATAATTAAAGATAAGGACTTCCTGGATAACGA | |
| | AGAGAATGAAGATATCTTAGAAGATATAGTGTTGACTCTTACCCTCTTTGAAGATCGGG | |
| | AAATGATTGAGGAAAGACTAAAAACATACGCTCACCTGTTCGACGATAAGGTTATGAAA | |
| | CAGTTAAAGAGGCGTCGCTATACGGGCTGGGGACGATTGTCGCGGAAACTTATCAACGG | |
| | GATAAGAGACAAGCAAAGTGGTAAAACTATTCTCGATTTTCTAAAGAGCGACGGCTTCG | |
| | CCAATAGGAACTTTATGCAGCTGATCCATGATGACTCTTTAACCTTCAAAGAGGATATA | |
| | CAAAAGGCACAGGTTTCCGGACAAGGGGACTCATTGCACGAACATATTGCGAATCTTGC | |
| | TGGTTCGCCAGCCATCAAAAAGGGCATACTCCAGACAGTCAAAGTAGTGGATGAGCTAG | |
| | TTAAGGTCATGGGACGTCACAAACCGGAAAACATTGTAATCGAGATGGCACGCGAAAAT | |
| | CAAACGACTCAGAAGGGGCAAAAAAACAGTCGAGAGCGGATGAAGAGAATAGAAGAGGG | |
| | TATTAAAGAACTGGGCAGCCAGATCTTAAAGGAGCATCCTGTGGAAAATACCCAATTGC | |
| | AGAACGAGAAACTTTACCTCTATTACCTACAAAATGGAAGGGACATGTATGTTGATCAG | |
| | GAACTGGACATAAACCGTTTATCTGATTACGACGTCGATCACATTGTACCCCAATCCTT | |
| | TTTGAAGGACGATTCAATCGACAATAAAGTGCTTACACGCTCGGATAAGAACCGAGGGA | |
| | AAAGTGACAATGTTCCAAGCGAGGAAGTCGTAAAGAAAATGAAGAACTATTGGCGGCAG | |
| | CTCCTAAATGCGAAACTGATAACGCAAAGAAAGTTCGATAACTTAACTAAAGCTGAGAG | |
| | GGGTGGCTTGTCTGAACTTGACAAGGCCGGATTTATTAAACGTCAGCTCGTGGAAACCC | |
| | GCCAAATCACAAAGCATGTTGCACAGATACTAGATTCCCGAATGAATACGAAATACGAC | |
| | GAGAACGATAAGCTGATTCGGGAAGTCAAAGTAATCACTTTAAAGTCAAAATTGGTGTC | |
| | GGACTTCAGAAAGGATTTTCAATTCTATAAAGTTAGGGAGATAAATAACTACCACCATG | |
| | CGCACGACGCTTATCTTAATGCCGTCGTAGGGACCGCACTCATTAAGAAATACCCGAAG | |
| | CTAGAAAGTGAGTTTGTGTATGGTGATTACAAAGTTTATGACGTCCGTAAGATGATCGC | |
| | GAAAAGCGAACAGGAGATAGGCAAGGCTACAGCCAAATACTTCTTTTATTCTAACATTA | |
| | TGAATTTCTTTAAGACGGAAATCACTCTGGCAAACGGAGAGATACGCAAACGACCTTTA | |
| | ATTGAAACCAATGGGGAGACAGGTGAAATCGTATGGGATAAGGGCCGGGACTTCGCGAC | |
| | GGTGAGAAAAGTTTTGTCCATGCCCCAAGTCAACATAGTAAAGAAAACTGAGGTGCAGA | |
| | CCGGAGGGTTTTCAAAGGAATCGATTCTTCCAAAAAGGAATAGTGATAAGCTCATCGCT | |
| | CGTAAAAAGGACTGGGACCCGAAAAAGTACGGTGGCTTCGATAGCCCTACAGTTGCCTA | |
| | TTCTGTCCTAGTAGTGGCAAAAGTTGAGAAGGGAAAATCCAAGAAACTGAAGTCAGTCA | |
| | AAGAATTATTGGGGATAACGATTATGGAGCGCTCGTCTTTTGAAAAGAACCCCATCGAC | |
| | TTCCTTGAGGCGAAAGGTTACAAGGAAGTAAAAAAGGATCTCATAATTAAACTACCAAA | |
| | GTATAGTCTGTTTGAGTTAGAAAATGGCCGAAAACGGATGTTGGCTAGCGCCGGAGAGC | |
| | TTCAAAAGGGGAACGAACTCGCACTACCGTCTAAATACGTGAATTTCCTGTATTTAGCG | |
| | TCCCATTACGAGAAGTTGAAAGGTTCACCTGAAGATAACGAACAGAAGCAACTTTTTGT | |
| | TGAGCAGCACAAACATTATCTCGACGAAATCATAGAGCAAATTTCGGAATTCAGTAAGA | |
| | GAGTCATCCTAGCTGATGCCAATCTGGACAAAGTATTAAGCGCATACAACAAGCACAGG | |
| | GATAAACCCATACGTGAGCAGGCGGAAAATATTATCCATTTGTTTACTCTTACCAACCT | |
| | CGGCGCTCCAGCCGCATTCAAGTATTTTGACACAACGATAGATCGCAAACGATACACTT | |
| | CTACCAAGGAGGTGCTAGACGCGACACTGATTCACCAATCCATCACGGGATTATATGAA | |
| | ACTCGGATAGATTTGTCACAGCTTGGGGGTGACGGATCCCCCAAGAAGAAGAGGAAAGT | |
| | CTCGAGCGACTACAAAGACCATGACGGTGATTATAAAGATCATGACATCGATTACAAGG | |
| | ATGACGATGACAAGGCTGCAGGA | |
| SpCas9 | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 331 |
| pyogenes wild | FGNIVDEVAYHEKYPTIYHLRKKLVDSTDKADLRLIYLALAHMIKFRGHFLIEGDLNPD | |
| type | NSDVDKLFIQLVQTYNQLFEENPINASGVDAKAILSARLSKSRRLENLIAQLPGEKKNG | |
| Encoded | LFGNLIALSLGLTPNFKSNFDLAEDAKLQLSKDTYDDDLDNLLAQIGDQYADLFLAAKN | |
| product of | LSDAILLSDILRVNTEITKAPLSASMIKRYDEHHQDLTLLKALVRQQLPEKYKEIFFDQ | |
| SWBC2D7W014 | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGDGSPKKKRKVSSDYKDHDGDYKDHDIDYKDDDDKAAG | |
| SpCas9 | ATGGATAAGAAATACTCAATAGGCTTAGATATCGGCACAAATAGCGTCGGATGGGCGGT | SEQ ID NO: |
| Streptococcus | GATCACTGATGAATATAAGGTTCCGTCTAAAAAGTTCAAGGTTCTGGGAAATACAGACC | 332 |
| pyogenes | GCCACAGTATCAAAAAAAATCTTATAGGGGCTCTTTTATTTGACAGTGGAGAGACAGCG | |
| M1 GAS wild | GAAGCGACTCGTCTCAAACGGACAGCTCGTAGAAGGTATACACGTCGGAAGAATCGTAT | |
| type | TTGTTATCTACAGGAGATTTTTTCAAATGAGATGGCGAAAGTAGATGATAGTTTCTTTC | |
| NC_002737.2 | ATCGACTTGAAGAGTCTTTTTTGGTGGAAGAAGACAAGAAGCATGAACGTCATCCTATT | |
| | TTTGGAAATATAGTAGATGAAGTTGCTTATCATGAGAAATATCCAACTATCTATCATCT | |
| | GCGAAAAAAATTGGTAGATTCTACTGATAAAGCGGATTTGCGCTTAATCTATTTGGCCT | |
| | TAGCGCATATGATTAAGTTTCGTGGTCATTTTTTGATTGAGGGAGATTTAAATCCTGAT | |
| | AATAGTGATGTGGACAAACTATTTATCCAGTTGGTACAAACCTACAATCAATTATTTGA | |
| | AGAAAACCCTATTAACGCAAGTGGAGTAGATGCTAAAGCGATTCTTTCTGCACGATTGA | |
| | GTAAATCAAGACGATTAGAAAATCTCATTGCTCAGCTCCCCGGTGAGAAGAAAAATGGC | |
| | TTATTTGGGAATCTCATTGCTTTGTCATTGGGTTTGACCCCTAATTTTAAATCAAATTT | |
| | TGATTTGGCAGAAGATGCTAAATTACAGCTTTCAAAAGATACTTACGATGATGATTTAG | |
| | ATAATTTATTGGCGCAAATTGGAGATCAATATGCTGATTTGTTTTTGGCAGCTAAGAAT | |
| | TTATCAGATGCTATTTTACTTTCAGATATCCTAAGAGTAAATACTGAAATAACTAAGGC | |
| | TCCCCTATCAGCTTCAATGATTAAACGCTACGATGAACATCATCAAGACTTGACTCTTT | |
| | TAAAAGCTTTAGTTCGACAACAACTTCCAGAAAAGTATAAAGAAATCTTTTTTGATCAA | |
| | TCAAAAAACGGATATGCAGGTTATATTGATGGGGGAGCTAGCCAAGAAGAATTTTATAA | |
| | ATTTATCAAACCAATTTTAGAAAAAATGGATGGTACTGAGGAATTATTGGTGAAACTAA | |
| | ATCGTGAAGATTTGCTGCGCAAGCAACGGACCTTTGACAACGGCTCTATTCCCCATCAA | |
| | ATTCACTTGGGTGAGCTGCATGCTATTTTGAGAAGACAAGAAGACTTTTATCCATTTTT | |
| | AAAAGACAATCGTGAGAAGATTGAAAAAATCTTGACTTTTCGAATTCCTTATTATGTTG | |
| | GTCCATTGGCGCGTGGCAATAGTCGTTTTGCATGGATGACTCGGAAGTCTGAAGAAACA | |
| | ATTACCCCATGGAATTTTGAAGAAGTTGTCGATAAAGGTGCTTCAGCTCAATCATTTAT | |
| | TGAACGCATGACAAACTTTGATAAAAATCTTCCAAATGAAAAAGTACTACCAAAACATA | |
| | GTTTGCTTTATGAGTATTTTACGGTTTATAACGAATTGACAAAGGTCAAATATGTTACT | |
| | GAAGGAATGCGAAAACCAGCATTTCTTTCAGGTGAACAGAAGAAAGCCATTGTTGATTT | |
| | ACTCTTCAAAACAAATCGAAAAGTAACCGTTAAGCAATTAAAAGAAGATTATTTCAAAA | |
| | AAATAGAATGTTTTGATAGTGTTGAAATTTCAGGAGTTGAAGATAGATTTAATGCTTCA | |
| | TTAGGTACCTACCATGATTTGCTAAAAATTATTAAAGATAAAGATTTTTTGGATAATGA | |
| | AGAAAATGAAGATATCTTAGAGGATATTGTTTTAACATTGACCTTATTTGAAGATAGGG | |
| | AGATGATTGAGGAAAGACTTAAAACATATGCTCACCTCTTTGATGATAAGGTGATGAAA | |
| | CAGCTTAAACGTCGCCGTTATACTGGTTGGGGACGTTTGTCTCGAAAATTGATTAATGG | |
| | TATTAGGGATAAGCAATCTGGCAAAACAATATTAGATTTTTTGAAATCAGATGGTTTTG | |
| | CCAATCGCAATTTTATGCAGCTGATCCATGATGATAGTTTGACATTTAAAGAAGACATT | |
| | CAAAAAGCACAAGTGTCTGGACAAGGCGATAGTTTACATGAACATATTGCAAATTTAGC | |
| | TGGTAGCCCTGCTATTAAAAAAGGTATTTTACAGACTGTAAAAGTTGTTGATGAATTGG | |
| | TCAAAGTAATGGGGCGGCATAAGCCAGAAAATATCGTTATTGAAATGGCACGTGAAAAT | |
| | CAGACAACTCAAAAGGGCCAGAAAAATTCGCGAGAGCGTATGAAACGAATCGAAGAAGG | |
| | TATCAAAGAATTAGGAAGTCAGATTCTTAAAGAGCATCCTGTTGAAAATACTCAATTGC | |
| | AAAATGAAAAGCTCTATCTCTATTATCTCCAAAATGGAAGAGACATGTATGTGGACCAA | |
| | GAATTAGATATTAATCGTTTAAGTGATTATGATGTCGATCACATTGTTCCACAAAGTTT | |
| | CCTTAAAGACGATTCAATAGACAATAAGGTCTTAACGCGTTCTGATAAAAATCGTGGTA | |
| | AATCGGATAACGTTCCAAGTGAAGAAGTAGTCAAAAAGATGAAAAACTATTGGAGACAA | |
| | CTTCTAAACGCCAAGTTAATCACTCAACGTAAGTTTGATAATTTAACGAAAGCTGAACG | |
| | TGGAGGTTTGAGTGAACTTGATAAAGCTGGTTTTATCAAACGCCAATTGGTTGAAACTC | |
| | GCCAAATCACTAAGCATGTGGCACAAATTTTGGATAGTCGCATGAATACTAAATACGAT | |
| | GAAAATGATAAACTTATTCGAGAGGTTAAAGTGATTACCTTAAAATCTAAATTAGTTTC | |
| | TGACTTCCGAAAAGATTTCCAATTCTATAAAGTACGTGAGATTAACAATTACCATCATG | |
| | CCCATGATGCGTATCTAAATGCCGTCGTTGGAACTGCTTTGATTAAGAAATATCCAAAA | |
| | CTTGAATCGGAGTTTGTCTATGGTGATTATAAAGTTTATGATGTTCGTAAAATGATTGC | |
| | TAAGTCTGAGCAAGAAATAGGCAAAGCAACCGCAAAATATTTCTTTTACTCTAATATCA | |
| | TGAACTTCTTCAAAACAGAAATTACACTTGCAAATGGAGAGATTCGCAAACGCCCTCTA | |
| | ATCGAAACTAATGGGGAAACTGGAGAAATTGTCTGGGATAAAGGGCGAGATTTTGCCAC | |
| | AGTGCGCAAAGTATTGTCCATGCCCCAAGTCAATATTGTCAAGAAAACAGAAGTACAGA | |
| | CAGGCGGATTCTCCAAGGAGTCAATTTTACCAAAAAGAAATTCGGACAAGCTTATTGCT | |
| | CGTAAAAAAGACTGGGATCCAAAAAAATATGGTGGTTTTGATAGTCCAACGGTAGCTTA | |
| | TTCAGTCCTAGTGGTTGCTAAGGTGGAAAAAGGGAAATCGAAGAAGTTAAAATCCGTTA | |
| | AAGAGTTACTAGGGATCACAATTATGGAAAGAAGTTCCTTTGAAAAAAATCCGATTGAC | |
| | TTTTTAGAAGCTAAAGGATATAAGGAAGTTAAAAAAGACTTAATCATTAAACTACCTAA | |
| | ATATAGTCTTTTTGAGTTAGAAAACGGTCGTAAACGGATGCTGGCTAGTGCCGGAGAAT | |
| | TACAAAAAGGAAATGAGCTGGCTCTGCCAAGCAAATATGTGAATTTTTTATATTTAGCT | |
| | AGTCATTATGAAAAGTTGAAGGGTAGTCCAGAAGATAACGAACAAAAACAATTGTTTGT | |
| | GGAGCAGCATAAGCATTATTTAGATGAGATTATTGAGCAAATCAGTGAATTTTCTAAGC | |
| | GTGTTATTTTAGCAGATGCCAATTTAGATAAAGTTCTTAGTGCATATAACAAACATAGA | |
| | GACAAACCAATACGTGAACAAGCAGAAAATATTATTCATTTATTTACGTTGACGAATCT | |
| | TGGAGCTCCCGCTGCTTTTAAATATTTTGATACAACAATTGATCGTAAACGATATACGT | |
| | CTACAAAAGAAGTTTTAGATGCCACTCTTATCCATCAATCCATCACTGGTCTTTATGAA | |
| | ACACGCATTGATTTGAGTCAGCTAGGAGGTGACTGA | |
| SpCas9 | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 324 |
| pyogenes | FGNIVDEVAYHEKYPTIYHLRKKLVDSTDKADLRLIYLALAHMIKFRGHFLIEGDLNPD | |
| M1 GAS wild | NSDVDKLFIQLVQTYNQLFEENPINASGVDAKAILSARLSKSRRLENLIAQLPGEKKNG | |
| type | LFGNLIALSLGLTPNFKSNFDLAEDAKLQLSKDTYDDDLDNLLAQIGDQYADLFLAAKN | |
| Encoded | LSDAILLSDILRVNTEITKAPLSASMIKRYDEHHQDLTLLKALVRQQLPEKYKEIFFDQ | |
| product of | SKNGYAGYIDGGASQEEFYKFIKPILEKMDGTEELLVKLNREDLLRKQRTFDNGSIPHQ | |
| NC_002737.2 | IHLGELHAILRRQEDFYPFLKDNREKIEKILTFRIPYYVGPLARGNSRFAWMTRKSEET | |
| (100% | ITPWNFEEVVDKGASAQSFIERMTNFDKNLPNEKVLPKHSLLYEYFTVYNELTKVKYVT | |
| identical to | EGMRKPAFLSGEQKKAIVDLLFKTNRKVTVKQLKEDYFKKIECFDSVEISGVEDRFNAS | |
| the canonical | LGTYHDLLKIIKDKDFLDNEENEDILEDIVLTLTLFEDREMIEERLKTYAHLFDDKVMK | |
| Q99ZW2 | QLKRRRYTGWGRLSRKLINGIRDKQSGKTILDFLKSDGFANRNFMQLIHDDSLTFKEDI | |
| wild type) | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |

The adenine base editors described herein may include any of the above SpCas9 sequences, or any variant thereof having at least 80%, at least 85%, at least 90%, at least 95%, or at least 99% sequence identity thereto.

B. Wild Type Cas9 Orthologs

In other embodiments, the Cas9 protein can be a wild type Cas9 ortholog from another bacterial species. For example, the following Cas9 orthologs can be used in connection with the adenine base editor constructs described in this disclosure. In addition, any variant Cas9 orthologs having at least 80%, at least 85%, at least 90%, at least 95%, or at least 99% sequence identity to any of the below orthologs may also be used with the disclosed adenine base editors.

| Description | Sequence | |
|---|---|---|
| LfCas 9 | 1 | MKEYHIGLDI GTSSIGWAVT DSQFKLMRIK GKTAIGVRLF EEGKTAAERR TFRTTRRRLK |
| Lactobacillus | 61 | RRKWRLHYLD EIFAPHLQEV DENFLRRLKQ SNIHPEDPTK NQAFIGKLLF PDLLKKNERG |
| fermentum | 121 | YPTLIKMRDE LPVEQRAHYP VMNIYKLREA MINEDRQFDL REVYLAVHHI VKYRGHFLNN |
| wild type | 181 | ASVDKFKVGR IDFDKSFNVL NEAYEELQNG EGSFTIEPSK VEKIGQLLLD TKMRKLDRQK |
| GenBank: | 241 | AVAKLLEVKV ADKEETKRNK QIATAMSKLV LGYKADFATV AMANGNEWKI DLSSETSEDE |
| SNX31424.1 1 | 301 | IEKFREELSD AQNDILTEIT SLFSQIMLNE IVPNGMSISE SMMDRYWTHE RQLAEVKEYL |
| | 361 | ATQPASARKE FDQVYNKYIG QAPKERGFDL EKGLKKILSK KENWKEIDEL LKAGDFLPKQ |
| | 421 | RTSANGVIPH QMHQQELDRI IEKQAKYYPW LATENPATGE RDRHQAKYEL DQLVSFRIPY |
| | 481 | YVGPLVTPEV QKATSGAKFA WAKRKEDGEI TPWNLWDKID RAESAEAFIK RMTVKDTYLL |
| | 541 | NEDVLPANSL LYQKYNVLNE LNNVRVNGRR LSVGIKQDIY TELFKKKKTV KASDVASLVM |
| | 601 | AKTRGVNKPS VEGLSDPKKF NSNLATYLDL KSIVGDKVDD NRYQTDLENI IEWRSVFEDG |
| | 661 | EIFADKLTEV EWLTDEQRSA LVKKRYKGWG RLSKKLLTGI VDENGQRIID LMWNTDQNFK |
| | 721 | EIVDQPVFKE QIDQLNQKAI TNDGMTLRER VESVLDDAYT SPQNKKAIWQ VVRVVEDIVK |
| | 781 | AVGNAPKSIS IEFARNEGNK GEITRSRRTQ LQKLFEDQAH ELVKDTSLTE ELEKAPDLSD |
| | 841 | RYYFYFTQGG KDMYTGDPIN FDEISTKYDI DHILPQSFVK DNSLDNRVLT SRKENNKKSD |
| | 901 | QVPAKLYAAK MKPYWNQLLK QGLITQRKFE NLTKDVDQNI KYRSLGFVKR QLVETRQVIK |
| | 961 | LTANILGSMY QEAGTEIIET RAGLTKQLRE EFDLPKVREV NDYHHAVDAY LTTFAGQYLN |
| | 1021 | RRYPKLRSFF VYGEYMKFKH GSDLKLRNFN FFHELMEGDK SQGKVVDQQT GELITTRDEV |
| | 1081 | AKSFDRLLNM KYMLVSKEVH DRSDQLYGAT IVTAKESGKL TSPIEIKKNR LVDLYGAYTN |
| | 1141 | GTSAFMTIIK FTGNKPKYKV IGIPTTSAAS LKRAGKPGSE SYNQELHRII KSNPKVKKGF |
| | 1201 | EIVVPHVSYG QLIVDGDCKF TLASPTVQHP ATQLVLSKKS LETISSGYKI LKDKPAIANE |
| | 1261 | RLIRVFDEVV GQMNRYFTIF DQRSNRQKVA DARDKFLSLP TESKYEGAKK VQVGKTEVIT |
| | 1321 | NLLMGLHANA TQGDLKVLGL ATFGFFQSTT GLSLSEDTMI VYQSPTGLFE RRICLKDI |
| | | (SEQ ID NO: 345) |
| SaCas 9 | MDKKYSIGLD IGTNSVGWAV ITDEYKVPSK KFKVLGNTDR HSIKKNLIGA LLFDSGETAE | |
| Staphylococcus | ATRLKRTARR RYTRRKNRIC YLQEIFSNEM AKVDDSFFHR LEESFLVEED KKHERHPIFG | |
| aureus wild | NIVDEVAYHE KYPTIYHLRK KLVDSTDKAD LRLIYLALAH MIKFRGHFLI EGDLNPDNSD | |
| type | VDKLFIQLVQ TYNQLFEENP INASGVDAKA ILSARLSKSR RLENLIAQLP GEKKNGLFGN | |
| GenBank: | LIALSLGLTP NFKSNFDLAE DAKLQLSKDT YDDDLDNLLA QIGDQYADLF LAAKNLSDAI | |
| AYD60528.1 | LLSDILRVNT EITKAPLSAS MIKRYDEHHQ DLTLLKALVR QQLPEKYKEI FFDQSKNGYA | |
| | GYIDGGASQE EFYKFIKPIL EKMDGTEELL VKLNREDLLR KQRTFDNGSI PHQIHLGELH | |
| | AILRRQEDFY PFLKDNREKI EKILTFRIPY YVGPLARGNS RFAWMTRKSE ETITPWNFEE | |
| | VVDKGASAQS FIERMTNFDK NLPNEKVLPK HSLLYEYFTV YNELTKVKYV TEGMRKPAFL | |
| | SGEQKKAIVD LLFKTNRKVT VKQLKEDYFK KIECFDSVEI SGVEDRFNAS LGTYHDLLKI | |
| | IKDKDFLDNE ENEDILEDIV LTLTLFEDRE MIEERLKTYA HLFDDKVMKQ LKRRRYTGWG | |
| | RLSRKLINGI RDKQSGKTIL DFLKSDGFAN RNFMQLIHDD SLTFKEDIQK AQVSGQGDSL | |
| | HEHIANLAGS PAIKKGILQT VKVVDELVKV MGRHKPENIV IEMARENQTT QKGQKNSRER | |
| | MKRIEEGIKE LGSQILKEHP VENTQLQNEK LYLYYLQNGR DMYVDQELDI NRLSDYDVDH | |
| | IVPQSFLKDD SIDNKVLTRS DKNRGKSDNV PSEEVVKKMK NYWRQLLNAK LITQRKFDNL | |
| | TKAERGGLSE LDKAGFIKRQ LVETRQITKH VAQILDSRMN TKYDENDKLI REVKVITLKS | |
| | KLVSDFRKDF QFYKVREINN YHHAHDAYLN AVVGTALIKK YPKLESEFVY GDYKVYDVRK | |
| | MIAKSEQEIG KATAKYFFYS NIMNFFKTEI TLANGEIRKR PLIETNGETG EIVWDKGRDF | |
| | ATVRKVLSMP QVNIVKKTEV QTGGFSKESI LPKRNSDKLI ARKKDWDPKK YGGFDSPTVA | |
| | YSVLVVAKVE KGKSKKLKSV KELLGITIME RSSFEKNPID FLEAKGYKEV KKDLIIKLPK | |
| | YSLFELENGR KRMLASAGEL QKGNELALPS KYVNFLYLAS HYEKLKGSPE DNEQKQLFVE | |
| | QHKHYLDEII EQISEFSKRV ILADANLDKV LSAYNKHRDK PIREQAENII HLFTLTNLGA | |
| | PAAFKYFDTT IDRKRYTSTK EVLDATLIHQ SITGLYETRI DLSQLGGD | |
| | (SEQ ID NO: 346) | |
| SaCas 9 | MGKRNYILGLDIGITSVGYGIIDYETRDVIDAGVRLFKEANVENNEGRRSKRGARRLKRRRRHRIQRVKKLL | |
| Staphylococcus | FDYNLLTDHSELSGINPYEARVKGLSQKLSEEEFSAALLHLAKRRGVHNVNEVEEDTGNELSTKEQISRNSK | |
| aureus | ALEEKYVAELQLERLKKDGEVRGSINRFKTSDYVKEAKQLLKVQKAYHQLDQSFIDTYIDLLETRRTYYEGP | |
| | GEGSPFGWKDIKEWYEMLMGHCTYFPEELRSVKYAYNADLYNALNDLNNLVITRDENEKLEYYEKFQIIENV | |
| | FKQKKKPTLKQIAKEILVNEEDIKGYRVTSTGKPEFTNLKVYHDIKDITARKEIIENAELLDQIAKILTIYQ | |
| | SSEDIQEELTNLNSELTQEEIEQISNLKGYTGTHNLSLKAINLILDELWHTNDNQIAIFNRLKLVPKKVDLS | |
| | QQKEIPTTLVDDFILSPVVKRSFIQSIKVINAIIKKYGLPNDIIIELAREKNSKDAQKMINEMQKRNRQTNE | |
| | RIEEIIRTTGKENAKYLIEKIKLHDMQEGKCLYSLEAIPLEDLLNNPFNYEVDHIIPRSVSFDNSFNNKVLV | |
| | KQEENSKKGNRTPFQYLSSSDSKISYETFKKHILNLAKGKGRISKTKKEYLLEERDINRFSVQKDFINRNLV | |
| | DTRYATRGLMNLLRSYFRVNNLDVKVKSINGGFTSFLRRKWKFKKERNKGYKHHAEDALIIANADFIFKEWK | |
| | KLDKAKKVMENQMFEEKQAESMPEIETEQEYKEIFITPHQIKHIKDFKDYKYSHRVDKKPNRKLINDTLYST | |
| | RKDDKGNTLIVNNLNGLYDKDNDKLKKLINKSPEKLLMYHHDPQTYQKLKLIMEQYGDEKNPLYKYYEETGN | |
| | YLTKYSKKDNGPVIKKIKYYGNKLNAHLDITDDYPNSRNKVVKLSLKPYRFDVYLDNGVYKFVTVKNLDVIK | |
| | KENYYEVNSKCYEEAKKLKKISNQAEFIASFYKNDLIKINGELYRVIGVNNDLLNRIEVNMIDITYREYLEN | |
| | MNDKRPPHIIKTIASKTQSIKKYSTDILGNLYEVKSKKHPQIIKK | |
| | (SEQ ID NO: 347) | |
| StCas9 | 1 | MLFNKCIIIS INLDFSNKEK CMTKPYSIGL DIGTNSVGWA VITDNYKVPS KKMKVLGNTS |
| Streptococcus | 61 | KKYIKKNLLG VLLFDSGITA EGRRLKRTAR RRYTRRRNRI LYLQEIFSTE MATLDDAFFQ |
| thermophilus | 121 | RLDDSFLVPD DKRDSKYPIF GNLVEEKVYH DEFPTIYHLR KYLADSTKKA DLRLVYLALA |
| UniProtKB/ | 181 | HMIKYRGHFL IEGEFNSKNN DIQKNFQDFL DTYNAIFESD LSLENSKQLE EIVKDKISKL |
| Swiss-Prot: | 241 | EKKDRILKLF PGEKNSGIFS EFLKLIVGNQ ADFRKCFNLD EKASLHFSKE SYDEDLETLL |
| G3ECR1.2 | 301 | GYIGDDYSDV FLKAKKLYDA ILLSGFLTVT DNETEAPLSS AMIKRYNEHK EDLALLKEYI |
| Wild type | 361 | RNISLKTYNE VFKDDTKNGY AGYIDGKTNQ EDFYVYLKNL LAEFEGADYF LEKIDREDFL |
| | 421 | RKQRTFDNGS IPYQIHLQEM RAILDKQAKF YPFLAKNKER IEKILTFRIP YYVGPLARGN |
| | 481 | SDFAWSIRKR NEKITPWNFE DVIDKESSAE AFINRMTSFD LYLPEEKVLP KHSLLYETFN |
| | 541 | VYNELTKVRF IAESMRDYQF LDSKQKKDIV RLYFKDKRKV TDKDIIEYLH AIYGYDGIEL |
| | 601 | KGIEKQFNSS LSTYHDLLNI INDKEFLDDS SNEAIIEEII HTLTIFEDRE MIKQRLSKFE |
| | 661 | NIFDKSVLKK LSRRHYTGWG KLSAKLINGI RDEKSGNTIL DYLIDDGISN RNFMQLIHDD |
| | 721 | ALSFKKKIQK AQIIGDEDKG NIKEVVKSLP GSPAIKKGIL QSIKIVDELV KVMGGRKPES |
| | 781 | IVVEMARENQ YTNQGKSNSQ QRLKRLEKSL KELGSKILKE NIPAKLSKID NNALQNDRLY |
| | 841 | LYYLQNGKDM YTGDDLDIDR LSNYDIDHII PQAFLKDNSI DNKVLVSSAS NRGKSDDFPS |
| | 901 | LEVVKKRKTF WYQLLKSKLI SQRKFDNLTK AERGGLLPED KAGFIQRQLV ETRQITKHVA |
| | 961 | RLLDEKFNNK KDENNRAVRT VKIITLKSTL VSQFRKDFEL YKVREINDFH HAHDAYLNAV |
| | 1021 | IASALLKKYP KLEPEFVYGD YPKYNSFRER KSATEKVYFY SNIMNIFKKS ISLADGRVIE |
| | 1081 | RPLIEVNEET GESVWNKESD LATVRRVLSY PQVNVVKKVE EQNHGLDRGK PKGLFNANLS |
| | 1141 | SKPKPNSNEN LVGAKEYLDP KKYGGYAGIS NSFAVLVKGT IEKGAKKKIT NVLEFQGISI |
| | 1201 | LDRINYRKDK LNFLLEKGYK DIELIIELPK YSLFELSDGS RRMLASILST NNKRGEIHKG |
| | 1261 | NQIFLSQKFV KLLYHAKRIS NTINENHRKY VENHKKEFEE LFYYILEFNE NYVGAKKNGK |
| | 1321 | LLNSAFQSWQ NHSIDELCSS FIGPTGSERK GLFELTSRGS AADFEFLGVK IPRYRDYTPS |
| | 1381 | SLLKDATLIH QSVTGLYETR IDLAKLGEG |
| | | (SEQ ID NO: 348) |
| LcCas 9 | 1 | MKIKNYNLAL TPSTSAVGHV EVDDDLNILE PVHHQKAIGV AKFGEGETAE ARRLARSARR |
| Lactobacillus | 61 | TTKRRANRIN HYFNEIMKPE IDKVDPLMFD RIKQAGLSPL DERKEFRTVI FDRPNIASYY |
| crispatus | 121 | HNQFPTIWHL QKYLMITDEK ADIRLIYWAL HSLLKHRGHF FNTTPMSQFK PGKLNLKDDM |
| NCBI Reference | 181 | LALDDYNDLE GLSFAVANSP EIEKVIKDRS MHKKEKIAEL KKLIVNDVPD KDLAKRNNKI |
| Sequence: | 241 | ITQIVNAIMG NSFHLNFIFD MDLDKLTSKA WSFKLDDPEL DTKFDAISGS MTDNQIGIFE |
| WP_133478044. | 301 | TLQKIYSAIS LLDILNGSSN VVDAKNALYD KHKRDLNLYF KFLNTLPDEI AKTLKAGYTL |
| 1 | 361 | YIGNRKKDLL AARKLLKVNV AKNFSQDDFY KLINKELKSI DKQGLQTRFS EKVGELVAQN |
| Wild type | 421 | NFLPVQRSSD NVFIPYQLNA ITFNKILENQ GKYYDFLVKP NPAKKDRKNA PYELSQLMQF |
| | 481 | TIPYYVGPLV TPEEQVKSGI PKTSRFAWMV RKDNGAITPW NFYDKVDIEA TADKFIKRSI |
| | 541 | AKDSYLLSEL VLPKHSLLYE KYEVFNELSN VSLDGKKLSG GVKQILFNEV FKKTNKVNTS |
| | 601 | RILKALAKHN IPGSKITGLS NPEEFTSSLQ TYNAWKKYFP NQIDNFAYQQ DLEKMIEWST |
| | 661 | VFEDHKILAK KLDEIEWLDD DQKKFVANTR LRGWGRLSKR LLTGLKDNYG KSIMQRLETT |
| | 721 | KANFQQIVYK PEFREQIDKI SQAAAKNQSL EDILANSYTS PSNRKAIRKT MSVVDEYIKL |
| | 781 | NHGKEPDKIF LMFQRSEQEK GKQTEARSKQ LNRILSQLKA DKSANKLFSK QLADEFSNAI |
| | 841 | KKSKYKLNDK QYFYFQQLGR DALTGEVIDY DELYKYTVLH IIPRSKLTDD SQNNKVLTKY |
| | 901 | KIVDGSVALK FGNSYSDALG MPIKAFWTEL NRLKLIPKGK LLNLTTDFST LNKYQRDGYI |
| | 961 | ARQLVETQQI VKLLATIMQS RFKHTKIIEV RNSQVANIRY QFDYFRIKNL NEYYRGFDAY |
| | 1021 | LAAVVGTYLY KVYPKARRLF VYGQYLKPKK TNQENQDMHL DSEKKSQGFN FLWNLLYGKQ |
| | 1081 | DQIFVNGTDV IAFNRKDLIT KMNTVYNYKS QKISLAIDYH NGAMFKATLE PRNDRDTAKT |
| | 1141 | RKLIPKKKDY DTDIYGGYTS NVDGYMLLAE IIKRDGNKQY GFYGVPSRLV SELDTLKKTR |
| | 1201 | YTEYEEKLKE IIKPELGVDL KKIKKIKILK NKVPFNQVII DKGSKFFITS TSYRWNYRQL |
| | 1261 | ILSAESQQTL MDLVVDPDFS NHKARKDARK NADERLIKVY EEILYQVKNY MPMFVELHRC |
| | 1321 | YEKLVDAQKT FKSLKISDKA MVLNQILILL HSNATSPVLE KLGYHTRFTL GKKHNLISEN |
| | 1381 | AVLVTQSITG LKENHVSIKQ ML |
| | | (SEQ ID NO: 349) |
| PdCas 9 | 1 | MTNEKYSIGL DIGTSSIGFA VVNDNNRVIR VKGKNAIGVR LFDEGKAAAD RRSFRTTRRS |
| Pedicoccus | 61 | FRTTRRRLSR RRWRLKLLRE IFDAYITPVD EAFFIRLKES NLSPKDSKKQ YSGDILFNDR |
| damnosus | 121 | SDKDFYEKYP TIYHLRNALM TEHRKFDVRE IYLAIHHIMK FRGHFLNATP ANNFKVGRLN |
| NCBI Reference | 181 | LEEKFEELND IYQRVFPDES IEFRTDNLEQ IKEVLLDNKR SRADRQRTLV SDIYQSSEDK |
| Sequence: | 241 | DIEKRNKAVA TEILKASLGN KAKLNVITNV EVDKEAAKEW SITFDSESID DDLAKIEGQM |
| WP_062913273. | 301 | TDDGHEIIEV LRSLYSGITL SAIVPENHTL SQSMVAKYDL HKDHLKLFKK LINGMTDTKK |
| 1 | 361 | AKNLRAAYDG YIDGVKGKVL PQEDFYKQVQ VNLDDSAEAN EIQTYIDQDI FMPKQRTKAN |
| Wild type | 421 | GSIPHQLQQQ ELDQIIENQK AYYPWLAELN PNPDKKRQQL AKYKLDELVT FRVPYYVGPM |
| | 481 | ITAKDQKNQS GAEFAWMIRK EPGNITPWNF DQKVDRMATA NQFIKRMTTT DTYLLGEDVL |
| | 541 | PAQSLLYQKF EVLNELNKIR IDHKPISIEQ KQQIFNDLFK QFKNVTIKHL QDYLVSQGQY |
| | 601 | SKRPLIEGLA DEKRFNSSLS TYSDLCGIFG AKLVEENDRQ EDLEKIIEWS TIFEDKKIYR |
| | 661 | AKLNDLTWLT DDQKEKLATK RYQGWGRLSR KLLVGLKNSE HRNIMDILWI TNENFMQIQA |
| | 721 | EPDFAKLVTD ANKGMLEKTD SQDVINDLYT SPQNKKAIRQ ILLVVHDIQN AMHGQAPAKI |
| | 781 | HVEFARGEER NPRRSVQRQR QVEAAYEKVS NELVSAKVRQ EFKEAINNKR DFKDRLFLYF |
| | 841 | MQGGIDIYTG KQLNIDQLSS YQIDHILPQA FVKDDSLTNR VLTNENQVKA DSVPIDIFGK |
| | 901 | KMLSVWGRMK DQGLISKGKY RNLTMNPENI SAHTENGFIN RQLVETRQVI KLAVNILADE |
| | 961 | YGDSTQIISV KADLSHQMRE DFELLKNRDV NDYHHAFDAY LAAFIGNYLL KRYPKLESYF |
| | 1021 | VYGDFKKFTQ KETKMRRFNF IYDLKHCDQV VNKETGEILW TKDEDIKYIR HLFAYKKILV |
| | 1081 | SHEVREKRGA LYNQTIYKAK DDKGSGQESK KLIRIKDDKE TKIYGGYSGK SLAYMTIVQI |
| | 1141 | TKKNKVSYRV IGIPTLALAR LNKLENDSTE NNGELYKIIK PQFTHYKVDK KNGEIIETTD |
| | 1201 | DFKIVVSKVR FQQLIDDAGQ FFMLASDTYK NNAQQLVISN NALKAINNTN ITDCPRDDLE |
| | 1261 | RLDNLRLDSA FDEIVKKMDK YFSAYDANNF REKIRNSNLI FYQLPVEDQW ENNKITELGK |
| | 1321 | RTVLTRILQG LHANATTTDM SIFKIKTPFG QLRQRSGISL SENAQLIYQS PTGLFERRVQ |
| | 1381 | LNKIK |
| | | (SEQ ID NO: 350) |
| FnCas 9 | 1 | MKKQKFSDYY LGFDIGTNSV GWCVTDLDYN VLRFNKKDMW GSRLFEEAKT AAERRVQRNS |
| Fusobacterium | 61 | RRRLKRRKWR LNLLEEIFSN EILKIDSNFF RRLKESSLWL EDKSSKEKFT LFNDDNYKDY |
| nucleatum | 121 | DFYKQYPTIF HLRNELIKNP EKKDIRLVYL AIHSIFKSRG HFLFEGQNLK EIKNFETLYN |
| NCBI Reference | 181 | NLIAFLEDNG INKIIDKNNI EKLEKIVCDS KKGLKDKEKE FKEIFNSDKQ LVAIFKLSVG |
| Sequence: | 241 | SSVSLNDLFD TDEYKKGEVE KEKISFREQI YEDDKPIYYS ILGEKIELLD IAKTFYDFMV |
| WP_060798984. | 301 | LNNILADSQY ISEAKVKLYE EHKKDLKNLK YIIRKYNKGN YDKLFKDKNE NNYSAYIGLN |
| 1 | 361 | KEKSKKEVIE KSRLKIDDLI KNIKGYLPKV EEIEEKDKAI FNKILNKIEL KTILPKQRIS |
| | 421 | DNGTLPYQIH EAELEKILEN QSKYYDFLNY EENGIITKDK LLMTFKFRIP YYVGPLNSYH |
| | 481 | KDKGGNSWIV RKEEGKILPW NFEQKVDIEK SAEEFIKRMT NKCTYLNGED VIPKDTFLYS |
| | 541 | EYVILNELNK VQVNDEFLNE ENKRKIIDEL FKENKKVSEK KFKEYLLVKQ IVDGTIELKG |
| | 601 | VKDSFNSNYI SYIRFKDIFG EKLNLDIYKE ISEKSILWKC LYGDDKKIFE KKIKNEYGDI |
| | 661 | LTKDEIKKIN TFKFNNWGRL SEKLLTGIEF INLETGECYS SVMDALRRTN YNLMELLSSK |
| | 721 | FTLQESINNE NKEMNEASYR DLIEESYVSP SLKRAIFQTL KIYEEIRKII GRVPKKVFIE |
| | 781 | MARGGDESMK NKKIPARQEQ LKKLYDSCGN DIANFSIDIK EMKNSLISYD NNSLRQKKLY |
| | 841 | LYYLQFGKCM YTGREIDLDR LLQNNDTYDI DHIYPRSKVI KDDSFDNLVL VLKNENAEKS |
| | 901 | NEYPVKKEIQ EKMKSFWRFL KEKNFISDEK YKRLTGKDDF ELRGFMARQL VNVRQTTKEV |
| | 961 | GKILQQIEPE IKIVYSKAEI ASSFREMFDF IKVRELNDTH HAKDAYLNIV AGNVYNTKFT |
| | 1021 | EKPYRYLQEI KENYDVKKIY NYDIKNAWDK ENSLEIVKKN MEKNTVNITR FIKEKKGQLF |
| | 1081 | DLNPIKKGET SNEIISIKPK VYNGKDDKLN EKYGYYKSLN PAYFLYVEHK EKNKRIKSFE |
| | 1141 | RVNLVDVNNI KDEKSLVKYL IENKKLVEPR VIKKVYKRQV ILINDYPYSI VTLDSNKLMD |
| | 1201 | FENLKPLFLE NKYEKILKNV IKFLEDNQGK SEENYKFIYL KKKDRYEKNE TLESVKDRYN |
| | 1261 | LEFNEMYDKF LEKLDSKDYK NYMNNKKYQE LLDVKEKFIK LNLFDKAFTL KSFLDLFNRK |
| | 1321 | TMADFSKVGL TKYLGKIQKI SSNVLSKNEL YLLEESVTGL FVKKIKL |
| | | (SEQ ID NO: 351) |
| EcCas 9 | 61 | RRKQRIQILQ ELLGEEVLKT DPGFFHRMKE SRYVVEDKRT LDGKQVELPY ALFVDKDYTD |
| Enterococcus | 121 | KEYYKQFPTI NHLIVYLMTT SDTPDIRLVY LALHYYMKNR GNFLHSGDIN NVKDINDILE |
| cecorum | 181 | QLDNVLETFL DGWNLKLKSY VEDIKNIYNR DLGRGERKKA FVNTLGAKTK |
| NCBI Reference | | AEKAFCSLIS |
| Sequence: | 241 | GGSTNLAELF DDSSLKEIET PKIEFASSSL EDKIDGIQEA LEDRFAVIEA |
| WP_047338501. | | AKRLYDWKTL |
| 1 | 301 | TDILGDSSSL AEARVNSYQM HHEQLLELKS LVKEYLDRKV FQEVFVSLNV |
| Wild type | | ANNYPAYIGH |
| | 361 | TKINGKKKEL EVKRTKRNDF YSYVKKQVIE PIKKKVSDEA VLTKLSEIES |
| | | LIEVDKYLPL |
| | 421 | QVNSDNGVIP YQVKLNELTR IFDNLENRIP VLRENRDKII KTFKFRIPYY |
| | | VGSLNGVVKN |
| | 481 | GKCTNWMVRK EEGKIYPWNF EDKVDLEASA EQFIRRMTNK CTYLVNEDVL |
| | | PKYSLLYSKY |
| | 541 | LVLSELNNLR IDGRPLDVKI KQDIYENVFK KNRKVTLKKI KKYLLKEGII |
| | | TDDDELSGLA |
| | 601 | DDVKSSLTAY RDFKEKLGHL DLSEAQMENI ILNITLFGDD KKLLKKRLAA |
| | | LYPFIDDKSL |
| | 661 | NRIATLNYRD WGRLSERFLS GITSVDQETG ELRTIIQCMY ETQANLMQLL |
| | | AEPYHFVEAI |
| | 721 | EKENPKVDLE SISYRIVNDL YVSPAVKRQI WQTLLVIKDI KQVMKHDPER |
| | | IFIEMAREKQ |
| | 781 | ESKKTKSRKQ VLSEVYKKAK EYEHLFEKLN SLTEEQLRSK KIYLYFTQLG |
| | | KCMYSGEPID |
| | 841 | FENLVSANSN YDIDHIYPQS KTIDDSFNNI VLVKKSLNAY KSNHYPIDKN |
| | | IRDNEKVKTL |
| | 901 | WNTLVSKGLI TKEKYERLIR STPFSDEELA GFIARQLVET RQSTKAVAEI |
| | | LSNWFPESEI |
| | 961 | VYSKAKNVSN FRQDFEILKV RELNDCHHAH DAYLNIVVGN AYHTKFTNSP |
| | | YRFIKNKANQ |
| | 1021 | EYNLRKLLQK VNKIESNGVV AWVGQSENNP GTIATVKKVI RRNTVLISRM |
| | | VKEVDGQLFD |
| | 1081 | LTLMKKGKGQ VPIKSSDERL TDISKYGGYN KATGAYFTFV KSKKRGKVVR |
| | | SFEYVPLHLS |
| | 1141 | KQFENNNELL KEYIEKDRGL TDVEILIPKV LINSLFRYNG SLVRITGRGD |
| | | TRLLLVHEQP |
| | 1201 | LYVSNSFVQQ LKSVSSYKLK KSENDNAKLT KTATEKLSNI DELYDGLLRK |
| | | LDLPIYSYWF |
| | 1261 | SSIKEYLVES RTKYIKLSIE EKALVIFEIL HLFQSDAQVP NLKILGLSTK |
| | | PSRIRIQKNL |
| | 1321 | KDTDKMSIIH QSPSGIFEHE IELTSL (SEQ ID NO: 352) |
| AhCas 9 | 1 | MQNGFLGITV SSEQVGWAVT NPKYELERAS RKDLWGVRLF DKAETAEDRR MFRTNRRLNQ |
| Anaerostipes | 61 | RKKNRIHYLR DIFHEEVNQK DPNFFQQLDE SNFCEDDRTV EFNFDTNLYK NQFPTVYHLR |
| hadrus | 121 | KYLMETKDKP DIRLVYLAFS KFMKNRGHFL YKGNLGEVMD FENSMKGFCE SLEKFNIDFP |
| NCBI Reference | 181 | TLSDEQVKEV RDILCDHKIA KTVKKKNIIT ITKVKSKTAK AWIGLFCGCS VPVKVLFQDI |
| Sequence: | 241 | DEEIVTDPEK ISFEDASYDD YIANIEKGVG IYYEAIVSAK MLFDWSILNE ILGDHQLLSD |
| WP_044924278. | 301 | AMIAEYNKHH DDLKRLQKII KGTGSRELYQ DIFINDVSGN YVCYVGHAKT MSSADQKQFY |
| 1 | 361 | TFLKNRLKNV NGISSEDAEW IDTEIKNGTL LPKQTKRDNS VIPHQLQLRE FELILDNMQE |
| Wild type | 421 | MYPFLKENRE KLLKIFNFVI PYYVGPLKGV VRKGESTNWM VPKKDGVIHP WNFDEMVDKE |
| | 481 | ASAECFISRM TGNCSYLFNE KVLPKNSLLY ETFEVLNELN PLKINGEPIS VELKQRIYEQ |
| | 541 | LFLTGKKVTK KSLTKYLIKN GYDKDIELSG IDNEFHSNLK SHIDFEDYDN LSDEEVEQII |
| | 601 | LRITVFEDKQ LLKDYLNREF VKLSEDERKQ ICSLSYKGWG NLSEMLLNGI TVTDSNGVEV |
| | 661 | SVMDMLWNTN LNLMQILSKK YGYKAEIEHY NKEHEKTIYN REDLMDYLNI PPAQRRKVNQ |
| | 721 | LITIVKSLKK TYGVPNKIFF KISREHQDDP KRTSSRKEQL KYLYKSLKSE DEKHLMKELD |
| | 781 | ELNDHELSND KVYLYFLQKG RCIYSGKKLN LSRLRKSNYQ NDIDYIYPLS AVNDRSMNNK |
| | 841 | VLTGIQENRA DKYTYFPVDS EIQKKMKGFW MELVLQGFMT KEKYFRLSRE NDFSKSELVS |
| | 901 | FIEREISDNQ QSGRMIASVL QYYFPESKIV FVKEKLISSF KRDFHLISSY GHNHLQAAKD |
| | 961 | AYITIVVGNV YHTKFTMDPA IYFKNHKRKD YDLNRLFLEN ISRDGQIAWE SGPYGSIQTV |
| | 1021 | RKEYAQNHIA VTKRVVEVKG GLFKQMPLKK GHGEYPLKTN DPRFGNIAQY GGYTNVTGSY |
| | 1081 | FVLVESMEKG KKRISLEYVP VYLHERLEDD PGHKLLKEYL VDHRKLNHPK ILLAKVRKNS |
| | 1141 | LLKIDGFYYR LNGRSGNALI LTNAVELIMD DWQTKTANKI SGYMKRRAID KKARVYQNEF |
| | 1201 | HIQELEQLYD FYLDKLKNGV YKNRKNNQAE LIHNEKEQFM ELKTEDQCVL LTEIKKLFVC |
| | 1261 | SPMQADLTLI GGSKHTGMIA MSSNVTKADF AVIAEDPLGL RNKVIYSHKG EK |
| | | (SEQ ID NO: 353) |
| KvCas 9 | 1 | MSQNNNKIYN IGLDIGDASV GWAVVDEHYN LLKRHGKHMW GSRLFTQANT AVERRSSRST |
| Kandleria | 61 | RRRYNKRRER IRLLREIMED MVLDVDPTFF IRLANVSFLD QEDKKDYLKE NYHSNYNLFI |
| vitulina | 121 | DKDFNDKTYY DKYPTIYHLR KHLCESKEKE DPRLIYLALH HIVKYRGNFL YEGQKFSMDV |
| NCBI Reference | 181 | SNIEDKMIDV LRQFNEINLF EYVEDRKKID EVLNVLKEPL SKKHKAEKAF ALFDTTKDNK |
| Sequence: | 241 | AAYKELCAAL AGNKFNVTKM LKEAELHDED EKDISFKFSD ATFDDAFVEK QPLLGDCVEF |
| WP_031589969. | 301 | IDLLHDIYSW VELQNILGSA HTSEPSISAA MIQRYEDHKN DLKLLKDVIR KYLPKKYFEV |
| 1 | 361 | FRDEKSKKNN YCNYINHPSK TPVDEFYKYI KKLIEKIDDP DVKTILNKIE LESFMLKQNS |
| Wild type | 421 | RTNGAVPYQM QLDELNKILE NQSVYYSDLK DNEDKIRSIL TFRIPYYFGP LNITKDRQFD |
| | 481 | WIIKKEGKEN ERILPWNANE IVDVDKTADE FIKRMRNFCT YFPDEPVMAK NSLTVSKYEV |
| | 541 | LNEINKLRIN DHLIKRDMKD KMLHTLFMDH KSISANAMKK WLVKNQYFSN TDDIKIEGFQ |
| | 601 | KENACSTSLT PWIDFTKIFG KINESNYDFI EKIIYDVTVF EDKKILRRRL KKEYDLDEEK |
| | 661 | IKKILKLKYS GWSRLSKKLL SGIKTKYKDS TRTPETVLEV MERTNMNLMQ VINDEKLGFK |
| | 721 | KTIDDANSTS VSGKFSYAEV QELAGSPAIK RGIWQALLIV DEIKKIMKHE PAHVYIEFAR |
| | 781 | NEDEKERKDS FVNQMLKLYK DYDFEDETEK EANKHLKGED AKSKIRSERL KLYYTQMGKC |
| | 841 | MYTGKSLDID RLDTYQVDHI VPQSLLKDDS IDNKVLVLSS ENQRKLDDLV IPSSIRNKMY |
| | 901 | GFWEKLFNNK IISPKKFYSL IKTEFNEKDQ ERFINRQIVE TRQITKHVAQ IIDNHYENTK |
| | 961 | VVTVRADLSH QFRERYHIYK NRDINDFHHA HDAYIATILG TYIGHRFESL DAKYIYGEYK |
| | 1021 | RIFRNQKNKG KEMKKNNDGF ILNSMRNIYA DKDTGEIVWD PNYIDRIKKC FYYKDCFVTK |
| | 1081 | KLEENNGTFF NVTVLPNDTN SDKDNTLATV PVNKYRSNVN KYGGFSGVNS FIVAIKGKKK |
| | 1141 | KGKKVIEVNK LTGIPLMYKN ADEEIKINYL KQAEDLEEVQ IGKEILKNQL IEKDGGLYYI |
| | 1201 | VAPTEIINAK QLILNESQTK LVCEIYKAMK YKNYDNLDSE KIIDLYRLLI NKMELYYPEY |
| | 1261 | RKQLVKKFED RYEQLKVISI EEKCNIIKQI LATLHCNSSI GKIMYSDFKI STTIGRLNGR |
| | 1321 | TISLDDISFI AESPTGMYSK KYKL (SEQ ID NO: 354) |
| EfCas 9 | 1 | MRLFEEGHTA EDRRLKRTAR RRISRRRNRL RYLQAFFEEA MTDLDENFFA RLQESFLVPE |
| Enterococcus | 61 | DKKWHRHPIF AKLEDEVAYH ETYPTIYHLR KKLADSSEQA DLRLIYLALA HIVKYRGHFL |
| faecalis | 121 | IEGKLSTENT SVKDQFQQFM VIYNQTFVNG ESRLVSAPLP ESVLIEEELT EKASRTKKSE |
| NCBI | 181 | KVLQQFPQEK ANGLFGQFLK LMVGNKADFK KVFGLEEEAK ITYASESYEE DLEGILAKVG |
| Reference | 241 | DEYSDVFLAA KNVYDAVELS TILADSDKKS HAKLSSSMIV RFTEHQEDLK KFKRFIRENC |
| Sequence: | 301 | PDEYDNLFKN EQKDGYAGYI AHAGKVSQLK FYQYVKKIIQ DIAGAEYFLE KIAQENFLRK |
| WP_016631044. | 361 | QRTFDNGVIP HQIHLAELQA IIHRQAAYYP FLKENQEKIE QLVTFRIPYY VGPLSKGDAS |
| 1 | 421 | TFAWLKRQSE EPIRPWNLQE TVDLDQSATA FIERMTNFDT YLPSEKVLPK HSLLYEKFMV |
| Wild type | 481 | FNELTKISYT DDRGIKANFS GKEKEKIFDY LFKTRRKVKK KDIIQFYRNE YNTEIVTLSG |
| | 541 | LEEDQFNASF STYQDLLKCG LTRAELDHPD NAEKLEDIIK ILTIFEDRQR IRTQLSTFKG |
| | 601 | QFSAEVLKKL ERKHYTGWGR LSKKLINGIY DKESGKTILD YLVKDDGVSK HYNRNFMQLI |
| | 661 | NDSQLSFKNA IQKAQSSEHE ETLSETVNEL AGSPAIKKGI YQSLKIVDEL VAIMGYAPKR |
| | 721 | IVVEMARENQ TTSTGKRRSI QRLKIVEKAM AEIGSNLLKE QPTTNEQLRD TRLFLYYMQN |
| | 781 | GKDMYTGDEL SLHRLSHYDI DHIIPQSFMK DDSLDNLVLV GSTENRGKSD DVPSKEVVKD |
| | 841 | MKAYWEKLYA AGLISQRKFQ RLTKGEQGGL TLEDKAHFIQ RQLVETRQIT KNVAGILDQR |
| | 901 | YNAKSKEKKV QIITLKASLT SQFRSIFGLY KVREVNDYHH GQDAYLNCVV ATTLLKVYPN |
| | 961 | LAPEFVYGEY PKFQTFKENK ATAKAIIYTN LLRFFTEDEP RFTKDGEILW SNSYLKTIKK |
| | 1021 | ELNYHQMNIV KKVEVQKGGF SKESIKPKGP SNKLIPVKNG LDPQKYGGFD SPVVAYTVLF |
| | 1081 | THEKGKKPLI KQEILGITIM EKTRFEQNPI LFLEEKGFLR PRVLMKLPKY TLYEFPEGRR |
| | 1141 | RLLASAKEAQ KGNQMVLPEH LLTLLYHAKQ CLLPNQSESL AYVEQHQPEF QEILERVVDF |
| | 1201 | AEVHTLAKSK VQQIVKLFEA NQTADVKEIA ASFIQLMQFN AMGAPSTFKF FQKDIERARY |
| | 1261 | TSIKEIFDAT IIYQSPTGLY ETRRKVVD (SEQ ID NO: 355) |

| | |
|---|---|
| Staphylococcus | KRNYILGLDIGITSVGYGIIDYETRDVIDAGVRLFKEANVENNEGRRSKRGARRLKRRRRHRIQRVKKLLFD |
| aureus Cas9 | YNLLTDHSELSGINPYEARVKGLSQKLSEEEFSAALLHLAKRRGVHNVNEVEEDTGNELSTKEQISRNSKAL |
| | EEKYVAELQLERLKKDGEVRGSINRFKTSDYVKEAKQLLKVQKAYHQLDQSFIDTYIDLLETRRTYYEGPGE |
| | GSPFGWKDIKEWYEMLMGHCTYFPEELRSVKYAYNADLYNALNDLNNLVITRDENEKLEYYEKFQIIENVFK |
| | QKKKPTLKQIAKEILVNEEDIKGYRVTSTGKPEFTNLKVYHDIKDITARKEIIENAELLDQIAKILTIYQSS |
| | EDIQEELTNLNSELTQEEIEQISNLKGYTGTHNLSLKAINLILDELWHTNDNQIAIFNRLKLVPKKVDLSQQ |
| | KEIPTTLVDDFILSPVVKRSFIQSIKVINAIIKKYGLPNDIIIELAREKNSKDAQKMINEMQKRNRQTNERI |
| | EEIIRTTGKENAKYLIEKIKLHDMQEGKCLYSLEAIPLEDLLNNPFNYEVDHIIPRSVSFDNSFNNKVLVKQ |
| | EENSKKGNRTPFQYLSSSDSKISYETFKKHILNLAKGKGRISKTKKEYLLEERDINRFSVQKDFINRNLVDT |
| | RYATRGLMNLLRSYFRVNNLDVKVKSINGGFTSFLRRKWKFKKERNKGYKHHAEDALIIANADFIFKEWKKL |
| | DKAKKVMENQMFEEKQAESMPEIETEQEYKEIFITPHQIKHIKDFKDYKYSHRVDKKPNRELINDTLYSTRK |
| | DDKGNTLIVNNLNGLYDKDNDKLKKLINKSPEKLLMYHHDPQTYQKLKLIMEQYGDEKNPLYKYYEETGNYL |
| | TKYSKKDNGPVIKKIKYYGNKLNAHLDITDDYPNSRNKVVKLSLKPYRFDVYLDNGVYKFVTVKNLDVIKKE |
| | NYYEVNSKCYEEAKKLKKISNQAEFIASFYNNDLIKINGELYRVIGVNNDLLNRIEVNMIDITYREYLENMN |
| | DKRPPRIIKTIASKTQSIKKYSTDILGNLYEVKSKKHPQIIKKG |
| | (SEQ ID NO: 356) |
| Geobacillus | MKYKIGLDIGITSIGWAVINLDIPRIEDLGVRIFDRAENPKTGESLALPRRLARSARRRLRRRKHRLERIRR |
| thermodenitri- | LFVREGILTKEELNKLFEKKHEIDVWQLRVEALDRKLNNDELARILLHLAKRRGFRSNRKSERTNKENSTML |
| ficans Cas9 | KHIEENQSILSSYRTVAEMVVKDPKFSLHKRNKEDNYTNTVARDDLEREIKLIFAKQREYGNIVCTEAFEHE |
| | YISIWASQRPFASKDDIEKKVGFCTFEPKEKRAPKATYTFQSFTVWEHINKLRLVSPGGIRALTDDERRLIY |
| | KQAFHKNKITFHDVRTLLNLPDDTRFKGLLYDRNTTLKENEKVRFLELGAYHKIRKAIDSVYGKGAAKSFRP |
| | IDFDTFGYALTMFKDDTDIRSYLRNEYEQNGKRMENLADKVYDEELIEELLNLSFSKFGHLSLKALRNILPY |
| | MEQGEVYSTACERAGYTFTGPKKKQKTVLLPNIPPIANPVVMRALTQARKVVNAIIKKYGSPVSIHIELARE |
| | LSQSFDERRKMQKEQEGNRKKNETAIRQLVEYGLTLNPTGLDIVKFKLWSEQNGKCAYSLQPIEIERLLEPG |
| | YTEVDHVIPYSRSLDDSYTNKVLVLTKENREKGNRTPAEYLGLGSERWQQFETFVLTNKQFSKKKRDRLLRL |
| | HYDENEENEFKNRNLNDTRYISRFLANFIREHLKFADSDDKQKVYTVNGRITAHLRSRWNFNKNREESNLHH |
| | AVDAAIVACTTPSDIARVTAFYQRREQNKELSKKTDPQFPQPWPHFADELQARLSKNPKESIKALNLGNYDN |
| | EKLESLQPVFVSRMPKRSITGAAHQETLRRYIGIDERSGKIQTVVKKKLSEIQLDKTGHFPMYGKESDPRTY |
| | EAIRQRLLEHNNDPKKAFQEPLYKPKKNGELGPIIRTIKIIDTTNQVIPLNDGKTVAYNSNIVRVDVFEKDG |
| | KYYCVPIYTIDMMKGILPNKAIEPNKPYSEWKEMTEDYTFRFSLYPNDLIRIEFPREKTIKTAVGEEIKIKD |
| | LFAYYQTIDSSNGGLSLVSHDNNFSLRSIGSRTLKRFEKYQVDVLGNIYKVRGEKRVGVASSSHSKAGETIR |
| | PL |
| | (SEQ ID NO: 357) |
| ScCas 9 | MEKKYSIGLDIGTNSVGWAVITDDYKVPSKKFKVLGNTNRKSIKKNLMGALLFDSGETAEATRLKRTARRRY |
| S. canis | TRRKNRIRYLQEIFANEMAKLDDSFFQRLEESFLVEEDKKNERHPIFGNLADEVAYHRNYPTIYHLRKKLAD |
| 1375 AA | SPEKADLRLIYLALAHIIKFRGHFLIEGKLNAENSDVAKLFYQLIQTYNQLFEESPLDEIEVDAKGILSARL |
| 159.2 kDa | SKSKRLEKLIAVFPNEKKNGLFGNIIALALGLTPNFKSNFDLTEDAKLQLSKDTYDDDLDELLGQIGDQYAD |
| | LFSAAKNLSDAILLSDILRSNSEVTKAPLSASMVKRYDEHHQDLALLKTLVRQQFPEKYAEIFKDDTKNGYA |
| | GYVGIGIKHRKRTTKLATQEEFYKFIKPILEKMDGAEELLAKLNRDDLLRKQRTFDNGSIPHQIHLKELHAI |
| | LRRQEEFYPFLKENREKIEKILTFRIPYYVGPLARGNSRFAWLTRKSEEAITPWNFEEVVDKGASAQSFIER |
| | MTNFDEQLPNKKVLPKHSLLYEYFTVYNELTKVKYVTERMRKPEFLSGEQKKAIVDLLFKTNRKVTVKQLKE |
| | DYFKKIECFDSVEIIGVEDRFNASLGTYHDLLKIIKDKDFLDNEENEDILEDIVLTLTLFEDREMIEERLKT |
| | YAHLFDDKVMKQLKRRHYTGWGRLSRKMINGIRDKQSGKTILDFLKSDGFSNRNFMQLIHDDSLTFKEEIEK |
| | AQVSGQGDSLHEQIADLAGSPAIKKGILQTVKIVDELVKVMGHKPENIVIEMARENQTTTKGLQQSRERKKR |
| | IEEGIKELESQILKENPVENTQLQNEKLYLYYLQNGRDMYVDQELDINRLSDYDVDHIVPQSFIKDDSIDNK |
| | VLTRSVENRGKSDNVPSEEVVKKMKNYWRQLLNAKLITQRKFDNLTKAERGGLSEADKAGFIKRQLVETRQI |
| | TKHVARILDSRMNTKRDKNDKPIREVKVITLKSKLVSDFRKDFQLYKVRDINNYHHAHDAYLNAVVGTALIK |
| | KYPKLESEFVYGDYKVYDVRKMIAKSEQEIGKATAKRFFYSNIMNFFKTEVKLANGEIRKRPLIETNGETGE |
| | VVWNKEKDFATVRKVLAMPQVNIVKKTEVQTGGFSKESILSKRESAKLIPRKKGWDTRKYGGFGSPTVAYSI |
| | LVVAKVEKGKAKKLKSVKVLVGITIMEKGSYEKDPIGFLEAKGYKDIKKELIFKLPKYSLFELENGRRRMLA |
| | SATELQKANELVLPQHLVRLLYYTQNISATTGSNNLGYIEQHREEFKEIFEKIIDFSEKYILKNKVNSNLKS |
| | SFDEQFAVSDSILLSNSFVSLLKYTSFGASGGFTFLDLDVKQGRLRYQTVTEVLDATLIYQSITGLYETRTD |
| | LSQLGGD (SEQ ID NO: 358) |

The adenine base editors described herein may include any of the above Cas9 ortholog sequences, or any variants thereof having at least 80%, at least 85%, at least 90%, at least 95%, or at least 99% sequence identity thereto.

The napDNAbp may include any suitable homologs and/or orthologs or naturally occurring enzymes, such as Cas9. Cas9 homologs and/or orthologs have been described in various species, including, but not limited to, S. pyogenes and S. thermophilus. Preferably, the Cas moiety is configured (e.g, mutagenized, recombinantly engineered, or otherwise obtained from nature) as a nickase, i.e., capable of cleaving only a single strand of the target doubpdditional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase. In some embodiments, the Cas9 protein comprises an amino acid sequence that is at least 80% identical to the amino acid sequence of a Cas9 protein as provided by any one of the variants of Table 3. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

C. Dead napDNAbp Variants

In some embodiments, the disclosed adenine base editors may comprise a catalytically inactive, or “dead,” napDNAbp domain. Exemplary catalytically inactive domains in the disclosed adenine base editors are dead S. pyogenes Cas9 (dSpCas9), dead S. aureus Cas9 (dSaCas9) and dead Lachnospiraceae bacterium Cas12a (dLbCas12a).

In certain embodiments, the adenine base editors described herein may include a dead Cas9, e.g., dead SpCas9, which has no nuclease activity due to one or more mutations that inactivate both nuclease domains of SpCas9, namely the RuvC domain (which cleaves the non-protospacer DNA strand) and HNH domain (which cleaves the protospacer DNA strand). The nuclease inactivation may be due to one or mutations that result in one or more substitutions and/or deletions in the amino acid sequence of the encoded protein, or any variants thereof having at least 80%, at least 85%, at least 90%, at least 95%, or at least 99% sequence identity thereto.

In certain embodiments, the adenine base editors described herein may include a dead Cas9, e.g., dead SpCas9, which has no nuclease activity due to one or more mutations that inactivate both nuclease domains of SaCas9, namely the RuvC domain (which cleaves the non-protospacer DNA strand) and HNH domain (which cleaves the protospacer DNA strand). The D10A and N580A mutations in the wild-type S. aureus Cas9 amino acid sequence may be used to form a dSaCas9. Accordingly, in some embodiments, the napDNAbp domain of the base editors provided herein comprises a dSaCas9 that has D10A and N580A mutations relative to the wild-type SaCas9 sequence (SEQ ID NO: 377).

As used herein, the term “dCas9” refers to a nuclease-inactive Cas9 or nuclease-dead Cas9, or a functional fragment thereof, and embraces any naturally occurring dCas9 from any organism, any naturally-occurring dCas9 equivalent or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

In other embodiments, dCas9 corresponds to, or comprises in part or in whole, a Cas9 amino acid sequence having one or more mutations that inactivate the Cas9 nuclease activity. In other embodiments, Cas9 variants having mutations other than D10A and H840A are provided which may result in the full or partial inactivate of the endogneous Cas9 nuclease activity (e.g., nCas9 or dCas9, respectively). Such mutations, by way of example, include other amino acid substitutions at D10 and H820, or other substitutions within the nuclease domains of Cas9 (e.g., substitutions in the HNH nuclease subdomain and/or the RuvC1 subdomain) with reference to a wild type sequence such as Cas9 from Streptococcus pyogenes (NCBI Reference Sequence: NC_017053.1). In some embodiments, variants or homologues of Cas9 (e.g., variants of Cas9 from Streptococcus pyogenes (NCBI Reference Sequence: NC_017053.1)) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g., variants of NCBI Reference Sequence: NC_017053.1) are provided having amino acid sequences which are shorter, or longer than NC_017053.1 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments, the napDNAbp domain of any of the disclosed base editors comprises a dead S. pyogenes Cas9 (dSpCas9). In some embodiments, the napDNAbp domain of any of the disclosed based editors is comprises at least 80%, at least 85%, at least 90%, at least 95%, or at least 99% sequence identity to SEQ ID NO: 360. In some embodiments, the napDNAbp domain of any of the disclosed base editors comprises the amino acid sequence of SEQ ID NO: 360.

In some embodiments, the napDNAbp domain of any of the disclosed base editors comprises a dead Lachnospiraceae bacterium Cas12a (dLbCas12a). In some embodiments, the napDNAbp domain of any of the disclosed based editors is comprises at least 80%, at least 85%, at least 90%, at least 95%, or at least 99% sequence identity to SEQ ID NO: 447. In some embodiments, the napDNAbp domain of any of the disclosed base editors comprises the amino acid sequence of SEQ ID NO: 447.

In one embodiment, the dead Cas9 may be based on the canonical SpCas9 sequence of Q99ZW2 and may have the following sequence, which comprises a D10A and an H810A substitutions (underlined and bolded), or a variant of SEQ ID NO: 359 having at least 80%, at least 85%, at least 90%, at least 95%, or at least 99% sequence identity thereto:

| Description | Sequence | SEQ ID NO: |
|---|---|---|
| dead Cas9 or | MDKKYSIGLXIGTNSVGWAVITDEYKVPSKKFKVLGNTDRHSIKKNLIGALLFDSGETA | SEQ ID NO: |
| dCas 9 | EATRLKRTARRRYTRRKNRICYLQEIFSNEMAKVDDSFFHRLEESFLVEEDKKHERHPI | 359 |
| Streptococcus | FGNIVDEVAYHEKYPTIYHLRKKLVDSTDKADLRLIYLALAHMIKFRGHFLIEGDLNPD | |
| pyogenes | NSDVDKLFIQLVQTYNQLFEENPINASGVDAKAILSARLSKSRRLENLIAQLPGEKKNG | |
| Q99ZW2 Cas9 | LFGNLIALSLGLTPNFKSNFDLAEDAKLQLSKDTYDDDLDNLLAQIGDQYADLFLAAKN | |
| with D10X and | LSDAILLSDILRVNTEITKAPLSASMIKRYDEHHQDLTLLKALVRQQLPEKYKEIFFDQ | |
| H810X | SKNGYAGYIDGGASQEEFYKFIKPILEKMDGTEELLVKLNREDLLRKQRTFDNGSIPHQ | |
| Where “X” is | IHLGELHAILRRQEDFYPFLKDNREKIEKILTFRIPYYVGPLARGNSRFAWMTRKSEET | |
| any amino | ITPWNFEEVVDKGASAQSFIERMTNFDKNLPNEKVLPKHSLLYEYFTVYNELTKVKYVT | |
| acid | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDXIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| dead Cas9 or | MDKKYSIGLAIGTNSVGWAVITDEYKVPSKKFKVLGNTDRHSIKKNLIGALLFDSGETA | SEQ ID NO: |
| dCas 9 | EATRLKRTARRRYTRRKNRICYLQEIFSNEMAKVDDSFFHRLEESFLVEEDKKHERHPI | 360 |
| Streptococcus | FGNIVDEVAYHEKYPTIYHLRKKLVDSTDKADLRLIYLALAHMIKFRGHFLIEGDLNPD | |
| pyogenes | NSDVDKLFIQLVQTYNQLFEENPINASGVDAKAILSARLSKSRRLENLIAQLPGEKKNG | |
| Q99ZW2 Cas9 | LFGNLIALSLGLTPNFKSNFDLAEDAKLQLSKDTYDDDLDNLLAQIGDQYADLFLAAKN | |
| with D10A and | LSDAILLSDILRVNTEITKAPLSASMIKRYDEHHQDLTLLKALVRQQLPEKYKEIFFDQ | |
| H810A | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDAIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| dead | MSKLEKFTNCYSLSKTLRFKAIPVGKTQENIDNKRLLVEDEKRAEDYKGVKKLLDRYYL | SEQ ID NO: |
| Lachnospiraceae | SFINDVLHSIKLKNLNNYISLFRKKTRTEKENKELENLEINLRKEIAKAFKGNEGYKSL | 447 |
| bacterium | FKKDIIETILPEFLDDKDEIALVNSFNGFTTAFTGFFDNRENMFSEEAKSTSIAFRCIN | |
| Cas12a | ENLTRYISNMDIFEKVDAIFDKHEVQEIKEKILNSDYDVEDFFEGEFFNFVLTQEGIDV | |
| | YNAIIGGFVTESGEKIKGLNEYINLYNQKTKQKLPKFKPLYKQVLSDRESLSFYGEGYT | |
| | SDEEVLEVFRNTLNKNSEIFSSIKKLEKLFKNFDEYSSAGIFVKNGPAISTISKDIFGE | |
| | WNVIRDKWNAEYDDIHLKKKAVVTEKYEDDRRKSFKKIGSFSLEQLQEYADADLSVVEK | |
| | LKEIIIQKVDEIYKVYGSSEKLFDADFVLEKSLKKNDAVVAIMKDLLDSVKSFENYIKA | |
| | FFGEGKETNRDESFYGDFVLAYDILLKVDHIYDAIRNYVTQKPYSKDKFKLYFQNPQFM | |
| | GGWDKDKETDYRATILRYGSKYYLAIMDKKYAKCLQKIDKDDVNGNYEKINYKLLPGPN | |
| | KMLPKVFFSKKWMAYYNPSEDIQKIYKNGTFKKGDMFNLNDCHKLIDFFKDSISRYPKW | |
| | SNAYDFNFSETEKYKDIAGFYREVEEQGYKVSFESASKKEVDKLVEEGKLYMFQIYNKD | |
| | FSDKSHGTPNLHTMYFKLLFDENNHGQIRLSGGAELFMRRASLKKEELVVHPANSPIAN | |
| | KNPDNPKKTTTLSYDVYKDKRFSEDQYELHIPIAINKCPKNIFKINTEVRVLLKHDDNP | |
| | YVIGIARGERNLLYIVVVDGKGNIVEQYSLNEIINNFNGIRIKTDYHSLLDKKEKERFE | |
| | ARQNWTSIENIKELKAGYISQVVHKICELVEKYDAVIALEDLNSGFKNSRVKVEKQVYQ | |
| | KFEKMLIDKLNYMVDKKSNPCATGGALKGYQITNKFESFKSMSTQNGFIFYIPAWLTSK | |
| | IDPSTGFVNLLKTKYTSIADSKKEISSFDRIMYVPEEDLFEFALDYKNFSRTDADYIKK | |
| | WKLYSYGNRIRIFRNPKKNNVFDWEEVCLTSAYKELFNKYGINYQQGDIRALLCEQSDK | |
| | AFYSSFMALMSLMLQMRNSITGRTDVDFLISPVKNSDGIFYDSRNYEAQENAILPKNAD | |
| | ANGAYNIARKVLWAIGQFKKAEDEKLDKVKIAISNKEWLEYAQTSVK | |

D. napDNAbp Nickase Variants

In some embodiments, the disclosed adenine base editors may comprise a napDNAbp domain that comprises a nickase. In some embodiments, the adenine base editors described herein comprise a Cas9 nickase. The term “Cas9 nickase” of “nCas9” refers to a variant of Cas9 which is capable of introducing a single-strand break in a double strand DNA molecule target. In some embodiments, the Cas9 nickase comprises only a single functioning nuclease domain. The wild type Cas9 (e.g., the canonical SpCas9) comprises two separate nuclease domains, namely, the RuvC domain (which cleaves the non-protospacer DNA strand) and HNH domain (which cleaves the protospacer DNA strand). In one embodiment, the Cas9 nickase comprises a mutation in the RuvC domain which inactivates the RuvC nuclease activity. For example, mutations in aspartate (D) 10, histidine (H) 983, aspartate (D) 986, or glutamate (E) 762, have been reported as loss-of-function mutations of the RuvC nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the RuvC domain could include D10X, H983X, D986X, or E762X, wherein X is any amino acid other than the wild type amino acid. In certain embodiments, the nickase could be D10A, of H983A, or D986A, or E762A, or a combination thereof.

In some embodiments, the napDNAbp domain of any of the disclosed base editors comprises an S. pyogenes Cas9 nickase (SpCas9n). In some embodiments, the napDNAbp domain of any of the disclosed based editors is comprises at least 80%, at least 85%, at least 90%, at least 95%, or at least 99% sequence identity to SEQ ID NO: 365 or 370. In some embodiments, the napDNAbp domain of any of the disclosed base editors comprises the amino acid sequence of SEQ ID NO: 365. In some embodiments, the napDNAbp domain of any of the disclosed base editors comprises the amino acid sequence of SEQ ID NO: 370.

In some embodiments, the napDNAbp domain of any of the disclosed base editors comprises an S. aureus Cas9 nickase (SaCas9n). In some embodiments, the napDNAbp domain of any of the disclosed based editors is comprises at least 80%, at least 85%, at least 90%, at least 95%, or at least 99% sequence identity to SEQ ID NO: 438. In some embodiments, the napDNAbp domain of any of the disclosed base editors comprises the amino acid sequence of SEQ ID NO: 438.

In various embodiments, the Cas9 nickase can having a mutation in the RuvC nuclease domain and have one of the following amino acid sequences, or a variant thereof having an amino acid sequence that has at least 80%, at least 85%, at least 90%, at least 95%, or at least 99% sequence identity thereto.

| Description | Sequence | SEQ ID NO: |
|---|---|---|
| Cas9 nickase | MDKKYSIGLXIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 361 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with D10X, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any alternate | SKNGYAGYIDGGASQEEFYKFIKPILEKMDGTEELLVKLNREDLLRKQRTFDNGSIPHQ | |
| amino acid | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 362 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with E762X, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any alternate | SKNGYAGYIDGGASQEEFYKFIKPILEKMDGTEELLVKLNREDLLRKQRTFDNGSIPHQ | |
| amino acid | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIXMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 363 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with H983X, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any alternate | SKNGYAGYIDGGASQEEFYKFIKPILEKMDGTEELLVKLNREDLLRKQRTFDNGSIPHQ | |
| amino acid | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHXAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 364 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with D986X, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any alternate | SKNGYAGYIDGGASQEEFYKFIKPILEKMDGTEELLVKLNREDLLRKQRTFDNGSIPHQ | |
| amino acid | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHXAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLAIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 365 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with D10A | LFGNLIALSLGLTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 366 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with E762A | LFGNLIALSLGLTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIAMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 367 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with H983A | LFGNLIALSLGLTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHAAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 368 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with D986A | LFGNLIALSLGLTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGSIPHQ | |
| | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHAAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MGKRNYILGLAIGITSVGYGIIDYETRDVIDAGVRLFKEANVENNEGRRSKRGARRLKRRRRHRIQR | SEQ ID NO: |
| Staphylococcus | VKKLLFDYNLLTDHSELSGINPYEARVKGLSQKLSEEEFSAALLHLAKRRGVHNVNEVEEDTGNELS | 438 |
| aureus | TKEQISRNSKALEEKYVAELQLERLKKDGEVRGSINRFKTSDYVKEAKQLLKVQKAYHQLDQSFIDT | |
| (SaCas9) | YIDLLETRRTYYEGPGEGSPFGWKDIKEWYEMLMGHCTYFPEELRSVKYAYNADLYNALNDLNNLVI | |
| with D10A | TRDENEKLEYYEKFQIIENVFKQKKKPTLKQIAKEILVNEEDIKGYRVTSTGKPEFTNLKVYHDIKD | |
| | ITARKEIIENAELLDQIAKILTIYQSSEDIQEELTNLNSELTQEEIEQISNLKGYTGTHNLSLKAIN | |
| | LILDELWHTNDNQIAIFNRLKLVPKKVDLSQQKEIPTTLVDDFILSPVVKRSFIQSIKVINAIIKKY | |
| | GLPNDIIIELAREKNSKDAQKMINEMQKRNRQTNERIEEIIRTTGKENAKYLIEKIKLHDMQEGKCL | |
| | YSLEAIPLEDLLNNPFNYEVDHIIPRSVSFDNSFNNKVLVKQEENSKKGNRTPFQYLSSSDSKISYE | |
| | TFKKHILNLAKGKGRISKTKKEYLLEERDINRFSVQKDFINRNLVDTRYATRGLMNLLRSYFRVNNL | |
| | DVKVKSINGGFTSFLRRKWKFKKERNKGYKHHAEDALIIANADFIFKEWKKLDKAKKVMENQMFEEK | |
| | QAESMPEIETEQEYKEIFITPHQIKHIKDFKDYKYSHRVDKKPNRKLINDTLYSTRKDDKGNTLIVN | |
| | NLNGLYDKDNDKLKKLINKSPEKLLMYHHDPQTYQKLKLIMEQYGDEKNPLYKYYEETGNYLTKYSK | |
| | KDNGPVIKKIKYYGNKLNAHLDITDDYPNSRNKVVKLSLKPYRFDVYLDNGVYKFVTVKNLDVIKKE | |
| | NYYEVNSKCYEEAKKLKKISNQAEFIASFYKNDLIKINGELYRVIGVNNDLLNRIEVNMIDITYREY | |
| | LENMNDKRPPHIIKTIASKTQSIKKYSTDILGNLYEVKSKKHPQIIKK | |

In another embodiment, the Cas9 nickase comprises a mutation in the HNH domain which inactivates the HNH nuclease activity. For example, mutations in histidine (H) 840 or asparagine (R) 863 have been reported as loss-of-function mutations of the HNH nuclease domain and the creation of a functional Cas9 nickase (e.g., Nishimasu et al., “Crystal structure of Cas9 in complex with guide RNA and target DNA,” Cell 156(5), 935-949, which is incorporated herein by reference). Thus, nickase mutations in the HNH domain could include H840X and R863X, wherein X is any amino acid other than the wild type amino acid. In certain embodiments, the nickase could be H840A or R863A or a combination thereof.

In various embodiments, the Cas9 nickase can have a mutation in the HNH nuclease domain and have one of the following amino acid sequences, or a variant thereof having an amino acid sequence that has at least 80%, at least 85%, at least 90%, at least 95%, or at least 99% sequence identity thereto.

| Description | Sequence | SEQ ID NO: |
|---|---|---|
| Cas9 nickase | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 369 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with H840X, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any alternate | SKNGYAGYIDGGASQEEFYKFIKPILEKMDGTEELLVKLNREDLLRKQRTFDNGSIPHQ | |
| amino acid | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDXIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 370 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with H840A, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any alternate | SKNGYAGYIDGGASQEEFYKFIKPILEKMDGTEELLVKLNREDLLRKQRTFDNGSIPHQ | |
| amino acid | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDAIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 371 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with R863X, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any alternate | SKNGYAGYIDGGASQEEFYKFIKPILEKMDGTEELLVKLNREDLLRKQRTFDNGSIPHQ | |
| amino acid | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNXGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |
| Cas9 nickase | MDKKYSIGLDIGTNSVGWAVITDEYKVPSKKFKVLGNTDRHSIKKNLIGALLFDSGETA | SEQ ID NO: |
| Streptococcus | EATRLKRTARRRYTRRKNRICYLQEIFSNEMAKVDDSFFHRLEESFLVEEDKKHERHPI | 372 |
| pyogenes | FGNIVDEVAYHEKYPTIYHLRKKLVDSTDKADLRLIYLALAHMIKFRGHFLIEGDLNPD | |
| Q99ZW2 Cas9 | NSDVDKLFIQLVQTYNQLFEENPINASGVDAKAILSARLSKSRRLENLIAQLPGEKKNG | |
| with R863A, | LFGNLIALSLGLTPNFKSNFDLAEDAKLQLSKDTYDDDLDNLLAQIGDQYADLFLAAKN | |
| wherein X is | LSDAILLSDILRVNTEITKAPLSASMIKRYDEHHQDLTLLKALVRQQLPEKYKEIFFDQ | |
| any alternate | SKNGYAGYIDGGASQEEFYKFIKPILEKMDGTEELLVKLNREDLLRKQRTFDNGSIPHQ | |
| amino acid | IHLGELHAILRRQEDFYPFLKDNREKIEKILTFRIPYYVGPLARGNSRFAWMTRKSEET | |
| | ITPWNFEEVVDKGASAQSFIERMTNFDKNLPNEKVLPKHSLLYEYFTVYNELTKVKYVT | |
| | EGMRKPAFLSGEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYAHLFDDKVMK | |
| | QLKRRRYTGWGRLSRKLINGIRDKQSGKTILDFLKSDGFANRNFMQLIHDDSLTFKEDI | |
| | QKAQVSGQGDSLHEHIANLAGSPAIKKGILQTVKVVDELVKVMGRHKPENIVIEMAREN | |
| | QTTQKGQKNSRERMKRIEEGIKELGSQILKEHPVENTQLQNEKLYLYYLQNGRDMYVDQ | |
| | ELDINRLSDYDVDHIVPQSFLKDDSIDNKVLTRSDKNAGKSDNVPSEEVVKKMKNYWRQ | |
| | LLNAKLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIKKYPK | |
| | LESEFVYGDYKVYDVRKMIAKSEQEIGKATAKYFFYSNIMNFFKTEITLANGEIRKRPL | |
| | IETNGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDKVLSAYNKHR | |
| | DKPIREQAENIIHLFTLTNLGAPAAFKYFDTTIDRKRYTSTKEVLDATLIHQSITGLYE | |
| | TRIDLSQLGGD | |

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

| Description | Sequence |
|---|---|
| Cas9 nickase | DKKYSIGLDIGTNSVGWAVITDEYKVPSKKFKVLGNTDRHSIKKNLIGALLFDSGETAEATRLKRTARRRYTR |
| (Met minus) | RKNRICYLQEIFSNEMAKVDDSFFHRLEESFLVEEDKKHERHPIFGNIVDEVAYHEKYPTIYHLRKKLVDSTD |
| Streptococcus | KADLRLIYLALAHMIKFRGHFLIEGDLNPDNSDVDKLFIQLVQTYNQLFEENPINASGVDAKAILSARLSKSR |
| pyogenes | RLENLIAQLPGEKKNGLFGNLIALSLGLTPNFKSNFDLAEDAKLQLSKDTYDDDLDNLLAQIGDQYADLFLAA |
| Q99ZW2 Cas9 | KNLSDAILLSDILRVNTEITKAPLSASMIKRYDEHHQDLTLLKALVRQQLPEKYKEIFFDQSKNGYAGYIDGG |
| with H840X, | ASQEEFYKFIKPILEKMDGTEELLVKLNREDLLRKQRTFDNGSIPHQIHLGELHAILRRQEDFYPFLKDNREK |
| wherein X is | IEKILTFRIPYYVGPLARGNSRFAWMTRKSEETITPWNFEEVVDKGASAQSFIERMTNFDKNLPNEKVLPKHS |
| any alternate | LLYEYFTVYNELTKVKYVTEGMRKPAFLSGEQKKAIVDLLFKTNRKVTVKQLKEDYFKKIECFDSVEISGVED |
| amino acid | RFNASLGTYHDLLKIIKDKDFLDNEENEDILEDIVLTLTLFEDREMIEERLKTYAHLFDDKVMKQLKRRRYTG |
| | WGRLSRKLINGIRDKQSGKTILDFLKSDGFANRNFMQLIHDDSLTFKEDIQKAQVSGQGDSLHEHIANLAGSP |
| | AIKKGILQTVKVVDELVKVMGRHKPENIVIEMARENQTTQKGQKNSRERMKRIEEGIKELGSQILKEHPVENT |
| | QLQNEKLYLYYLQNGRDMYVDQELDINRLSDYDVDXIVPQSFLKDDSIDNKVLTRSDKNRGKSDNVPSEEVVK |
| | KMKNYWRQLLNAKLITQRKFDNLTKAERGGLSELDKAGFIKRQLVETRQITKHVAQILDSRMNTKYDENDKLI |
| | REVKVITLKSKLVSDFRKDFQFYKVREINNYHHAHDAYLNAVVGTALIKKYPKLESEFVYGDYKVYDVRKMIA |
| | KSEQEIGKATAKYFFYSNIMNFFKTEITLANGEIRKRPLIETNGETGEIVWDKGRDFATVRKVLSMPQVNIVK |
| | KTEVQTGGFSKESILPKRNSDKLIARKKDWDPKKYGGFDSPTVAYSVLVVAKVEKGKSKKLKSVKELLGITIM |
| | ERSSFEKNPIDFLEAKGYKEVKKDLIIKLPKYSLFELENGRKRMLASAGELQKGNELALPSKYVNFLYLASHY |
| | EKLKGSPEDNEQKQLFVEQHKHYLDEIIEQISEFSKRVILADANLDKVLSAYNKHRDKPIREQAENIIHLFTL |
| | TNLGAPAAFKYFDTTIDRKRYTSTKEVLDATLIHQSITGLYETRIDLSQLGGD (SEQ ID NO: 373) |
| Cas9 nickase | DKKYSIGLDIGTNSVGWAVITDEYKVPSKKFKVLGNTDRHSIKKNLIGALLFDSGETAEATRLKRTARRRYTR |
| (Met minus) | RKNRICYLQEIFSNEMAKVDDSFFHRLEESFLVEEDKKHERHPIFGNIVDEVAYHEKYPTIYHLRKKLVDSTD |
| Streptococcus | KADLRLIYLALAHMIKFRGHFLIEGDLNPDNSDVDKLFIQLVQTYNQLFEENPINASGVDAKAILSARLSKSR |
| pyogenes | RLENLIAQLPGEKKNGLFGNLIALSLGLTPNFKSNFDLAEDAKLQLSKDTYDDDLDNLLAQIGDQYADLFLAA |
| Q99ZW2 Cas9 | KNLSDAILLSDILRVNTEITKAPLSASMIKRYDEHHQDLTLLKALVRQQLPEKYKEIFFDQSKNGYAGYIDGG |
| with H840A, | ASQEEFYKFIKPILEKMDGTEELLVKLNREDLLRKQRTFDNGSIPHQIHLGELHAILRRQEDFYPFLKDNREK |
| wherein X is | IEKILTFRIPYYVGPLARGNSRFAWMTRKSEETITPWNFEEVVDKGASAQSFIERMTNFDKNLPNEKVLPKHS |
| any alternate | LLYEYFTVYNELTKVKYVTEGMRKPAFLSGEQKKAIVDLLFKTNRKVTVKQLKEDYFKKIECFDSVEISGVED |
| amino acid | RFNASLGTYHDLLKIIKDKDFLDNEENEDILEDIVLTLTLFEDREMIEERLKTYAHLFDDKVMKQLKRRRYTG |
| | WGRLSRKLINGIRDKQSGKTILDFLKSDGFANRNFMQLIHDDSLTFKEDIQKAQVSGQGDSLHEHIANLAGSP |
| | AIKKGILQTVKVVDELVKVMGRHKPENIVIEMARENQTTQKGQKNSRERMKRIEEGIKELGSQILKEHPVENT |
| | QLQNEKLYLYYLQNGRDMYVDQELDINRLSDYDVDAIVPQSFLKDDSIDNKVLTRSDKNRGKSDNVPSEEVVK |
| | KMKNYWRQLLNAKLITQRKFDNLTKAERGGLSELDKAGFIKRQLVETRQITKHVAQILDSRMNTKYDENDKLI |
| | REVKVITLKSKLVSDFRKDFQFYKVREINNYHHAHDAYLNAVVGTALIKKYPKLESEFVYGDYKVYDVRKMIA |
| | KSEQEIGKATAKYFFYSNIMNFFKTEITLANGEIRKRPLIETNGETGEIVWDKGRDFATVRKVLSMPQVNIVK |
| | KTEVQTGGFSKESILPKRNSDKLIARKKDWDPKKYGGFDSPTVAYSVLVVAKVEKGKSKKLKSVKELLGITIM |
| | ERSSFEKNPIDFLEAKGYKEVKKDLIIKLPKYSLFELENGRKRMLASAGELQKGNELALPSKYVNFLYLASHY |
| | EKLKGSPEDNEQKQLFVEQHKHYLDEIIEQISEFSKRVILADANLDKVLSAYNKHRDKPIREQAENIIHLFTL |
| | TNLGAPAAFKYFDTTIDRKRYTSTKEVLDATLIHQSITGLYETRIDLSQLGGD (SEQ ID NO: 374) |
| Cas9 nickase | DKKYSIGLDIGTNSVGWAVITDEYKVPSKKFKVLGNTDRHSIKKNLIGALLFDSGETAEATRLKRTARRRYTR |
| (Met minus) | RKNRICYLQEIFSNEMAKVDDSFFHRLEESFLVEEDKKHERHPIFGNIVDEVAYHEKYPTIYHLRKKLVDSTD |
| Streptococcus | KADLRLIYLALAHMIKFRGHFLIEGDLNPDNSDVDKLFIQLVQTYNQLFEENPINASGVDAKAILSARLSKSR |
| pyogenes | RLENLIAQLPGEKKNGLFGNLIALSLGLTPNFKSNFDLAEDAKLQLSKDTYDDDLDNLLAQIGDQYADLFLAA |
| Q99ZW2 Cas9 | KNLSDAILLSDILRVNTEITKAPLSASMIKRYDEHHQDLTLLKALVRQQLPEKYKEIFFDQSKNGYAGYIDGG |
| with R863X, | ASQEEFYKFIKPILEKMDGTEELLVKLNREDLLRKQRTFDNGSIPHQIHLGELHAILRRQEDFYPFLKDNREK |
| wherein X is | IEKILTFRIPYYVGPLARGNSRFAWMTRKSEETITPWNFEEVVDKGASAQSFIERMTNFDKNLPNEKVLPKHS |
| any alternate | LLYEYFTVYNELTKVKYVTEGMRKPAFLSGEQKKAIVDLLFKTNRKVTVKQLKEDYFKKIECFDSVEISGVED |
| amino acid | RFNASLGTYHDLLKIIKDKDFLDNEENEDILEDIVLTLTLFEDREMIEERLKTYAHLFDDKVMKQLKRRRYTG |
| | WGRLSRKLINGIRDKQSGKTILDFLKSDGFANRNFMQLIHDDSLTFKEDIQKAQVSGQGDSLHEHIANLAGSP |
| | AIKKGILQTVKVVDELVKVMGRHKPENIVIEMARENQTTQKGQKNSRERMKRIEEGIKELGSQILKEHPVENT |
| | QLQNEKLYLYYLQNGRDMYVDQELDINRLSDYDVDHIVPQSFLKDDSIDNKVLTRSDKNXGKSDNVPSEEVVK |
| | KMKNYWRQLLNAKLITQRKFDNLTKAERGGLSELDKAGFIKRQLVETRQITKHVAQILDSRMNTKYDENDKLI |
| | REVKVITLKSKLVSDFRKDFQFYKVREINNYHHAHDAYLNAVVGTALIKKYPKLESEFVYGDYKVYDVRKMIA |
| | KSEQEIGKATAKYFFYSNIMNFFKTEITLANGEIRKRPLIETNGETGEIVWDKGRDFATVRKVLSMPQVNIVK |
| | KTEVQTGGFSKESILPKRNSDKLIARKKDWDPKKYGGFDSPTVAYSVLVVAKVEKGKSKKLKSVKELLGITIM |
| | ERSSFEKNPIDFLEAKGYKEVKKDLIIKLPKYSLFELENGRKRMLASAGELQKGNELALPSKYVNFLYLASHY |
| | EKLKGSPEDNEQKQLFVEQHKHYLDEIIEQISEFSKRVILADANLDKVLSAYNKHRDKPIREQAENIIHLFTL |
| | TNLGAPAAFKYFDTTIDRKRYTSTKEVLDATLIHQSITGLYETRIDLSQLGGD(SEQ ID NO: 375) |
| Cas9 nickase | DKKYSIGLDIGTNSVGWAVITDEYKVPSKKFKVLGNTDRHSIKKNLIGALLFDSGETAEATRLKRTARRRYTR |
| (Met minus) | RKNRICYLQEIFSNEMAKVDDSFFHRLEESFLVEEDKKHERHPIFGNIVDEVAYHEKYPTIYHLRKKLVDSTD |
| Streptococcus | KADLRLIYLALAHMIKFRGHFLIEGDLNPDNSDVDKLFIQLVQTYNQLFEENPINASGVDAKAILSARLSKSR |
| pyogenes | RLENLIAQLPGEKKNGLFGNLIALSLGLTPNFKSNFDLAEDAKLQLSKDTYDDDLDNLLAQIGDQYADLFLAA |
| Q99ZW2 Cas9 | KNLSDAILLSDILRVNTEITKAPLSASMIKRYDEHHQDLTLLKALVRQQLPEKYKEIFFDQSKNGYAGYIDGG |
| with R863A, | ASQEEFYKFIKPILEKMDGTEELLVKLNREDLLRKQRTFDNGSIPHQIHLGELHAILRRQEDFYPFLKDNREK |
| wherein X is | IEKILTFRIPYYVGPLARGNSRFAWMTRKSEETITPWNFEEVVDKGASAQSFIERMTNFDKNLPNEKVLPKHS |
| any alternate | LLYEYFTVYNELTKVKYVTEGMRKPAFLSGEQKKAIVDLLFKTNRKVTVKQLKEDYFKKIECFDSVEISGVED |
| amino acid | RFNASLGTYHDLLKIIKDKDFLDNEENEDILEDIVLTLTLFEDREMIEERLKTYAHLFDDKVMKQLKRRRYTG |
| | WGRLSRKLINGIRDKQSGKTILDFLKSDGFANRNFMQLIHDDSLTFKEDIQKAQVSGQGDSLHEHIANLAGSP |
| | AIKKGILQTVKVVDELVKVMGRHKPENIVIEMARENQTTQKGQKNSRERMKRIEEGIKELGSQILKEHPVENT |
| | QLQNEKLYLYYLQNGRDMYVDQELDINRLSDYDVDHIVPQSFLKDDSIDNKVLTRSDKNAGKSDNVPSEEVVK |
| | KMKNYWRQLLNAKLITQRKFDNLTKAERGGLSELDKAGFIKRQLVETRQITKHVAQILDSRMNTKYDENDKLI |
| | REVKVITLKSKLVSDFRKDFQFYKVREINNYHHAHDAYLNAVVGTALIKKYPKLESEFVYGDYKVYDVRKMIA |
| | KSEQEIGKATAKYFFYSNIMNFFKTEITLANGEIRKRPLIETNGETGEIVWDKGRDFATVRKVLSMPQVNIVK |
| | KTEVQTGGFSKESILPKRNSDKLIARKKDWDPKKYGGFDSPTVAYSVLVVAKVEKGKSKKLKSVKELLGITIM |
| | ERSSFEKNPIDFLEAKGYKEVKKDLIIKLPKYSLFELENGRKRMLASAGELQKGNELALPSKYVNFLYLASHY |
| | EKLKGSPEDNEQKQLFVEQHKHYLDEIIEQISEFSKRVILADANLDKVLSAYNKHRDKPIREQAENIIHLFTL |
| | TNLGAPAAFKYFDTTIDRKRYTSTKEVLDATLIHQSITGLYETRIDLSQLGGD (SEQ ID NO: 376) |

E. Other Cas9 Variants

The napDNAbp domains used in the base editors described herein may also include other Cas9 variants that area at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SEQ ID NO: 326).

In some embodiments, the disclosure also may utilize Cas9 fragments which retain their functionality and which are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In various embodiments, the adenine base editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

F. Other Cas9 Equivalents

In some embodiments, the adenine base editors described herein can include any Cas9 equivalent. As used herein, the term “Cas9 equivalent” is a broad term that encompasses any napDNAbp protein that serves the same function as Cas9 in the present adenine base editors despite that its amino acid primary sequence and/or its three-dimensional structure may be different and/or unrelated from an evolutionary standpoint. Thus, while Cas9 equivalents include any Cas9 ortholog, homolog, mutant, or variant described or embraced herein that are evolutionarily related, the Cas9 equivalents also embrace proteins that may have evolved through convergent evolution processes to have the same or similar function as Cas9, but which do not necessarily have any similarity with regard to amino acid sequence and/or three dimensional structure. The adenine base editors described here embrace any Cas9 equivalent that would provide the same or similar function as Cas9 despite that the Cas9 equivalent may be based on a protein that arose through convergent evolution.

For example, CasX is a Cas9 equivalent that reportedly has the same function as Cas9 but which evolved through convergent evolution. Thus, the CasX protein described in Liu et al., “CasX enzymes comprises a distinct family of RNA-guided genome editors,” Nature, 2019, Vol. 566: 218-223, is contemplated to be used with the adenine base editors described herein. In addition, any variant or modification of CasX is conceivable and within the scope of the present disclosure.

Cas9 is a bacterial enzyme that evolved in a wide variety of species. However, the Cas9 equivalents contemplated herein may also be obtained from archaea, which constitute a domain and kingdom of single-celled prokaryotic microbes different from bacteria.

In some embodiments, Cas9 equivalents may refer to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure. Also see Liu et al., “CasX enzymes comprises a distinct family of RNA-guided genome editors,” Nature, 2019, Vol. 566: 218-223. Any of these Cas9 equivalents are contemplated.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein. In some embodiments, the napDNAbp is a naturally-occurring CasX or CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), CasX, CasY, Cpf1, C2c1, C2c2, C2C3, Argonaute, Cas12a, and Cas12b. One example of a nucleic acid programmable DNA-binding protein that has different PAM specificity than Cas9 is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference. The state of the art may also now refer to Cpf1 enzymes as Cas12a.

In still other embodiments, the Cas protein may include any CRISPR associated protein, including but not limited to, Cas12a, Cas12b, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type SpCas9 polypeptide of SEQ ID NO: 326).

In various other embodiments, the napDNAbp can be any of the following proteins: a Cas9, a Cpf1, a CasX, a CasY, a C2c1, a C2c2, a C2c3, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, a circularly permuted Cas9 domain such as CP1012, CP1028, CP1041, CP1249, and CP1300, or an Argonaute (Ago) domain, a Cas9-KKH, a SmacCas9, a Spy-macCas9, an SpCas9-VRQR, an SpCas9-VRER, an SpCas9-VQR, an SpCas9-EQR, an SpCas9-NRRH, an SpaCas9-NRTH, an SpCas9-NRCH, an LbCas12a, an AsCas12a, a CeCas12a, an MbCas12a, a CasΦ, an SpCas9-NG-CP1041, an SpCas9-NG-VRQR, or a variant thereof.

In certain embodiments, the adenine base editors contemplated herein can include a Cas9 protein that is of smaller molecular weight than the canonical SpCas9 sequence. In some embodiments, the smaller-sized Cas9 variants may facilitate delivery to cells, e.g., by an expression vector, nanoparticle, or other means of delivery. The canonical SpCas9 protein is 1368 amino acids in length and has a predicted molecular weight of 158 kilodaltons. The term “small-sized Cas9 variant”, as used herein, refers to any Cas9 variant—naturally occurring, engineered, or otherwise—that is less than at least 1300 amino acids, or at least less than 1290 amino acids, or than less than 1280 amino acids, or less than 1270 amino acid, or less than 1260 amino acid, or less than 1250 amino acids, or less than 1240 amino acids, or less than 1230 amino acids, or less than 1220 amino acids, or less than 1210 amino acids, or less than 1200 amino acids, or less than 1190 amino acids, or less than 1180 amino acids, or less than 1170 amino acids, or less than 1160 amino acids, or less than 1150 amino acids, or less than 1140 amino acids, or less than 1130 amino acids, or less than 1120 amino acids, or less than 1110 amino acids, or less than 1100 amino acids, or less than 1050 amino acids, or less than 1000 amino acids, or less than 950 amino acids, or less than 900 amino acids, or less than 850 amino acids, or less than 800 amino acids, or less than 750 amino acids, or less than 700 amino acids, or less than 650 amino acids, or less than 600 amino acids, or less than 550 amino acids, or less than 500 amino acids, but at least larger than about 400 amino acids and retaining the required functions of the Cas9 protein.

In various embodiments, the adenine base editors disclosed herein may comprise one of the small-sized Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference small-sized Cas9 protein. Exemplary small-sized Cas9 variants include, but are not limited to, SaCas9 and LbCas12a.

In some embodiments, the napDNAbp domain of any of the disclosed base editors comprises an LbCas12a, such as a wild-type LbCas12a. In some embodiments, the napDNAbp domain of any of the disclosed based editors is comprises at least 80%, at least 85%, at least 90%, at least 95%, or at least 99% sequence identity to SEQ ID NO: 381. In some embodiments, the napDNAbp domain of any of the disclosed base editors comprises the amino acid sequence of SEQ ID NO: 381.

In some embodiments, the napDNAbp domain of any of the disclosed base editors comprises an AsCas12a, such as a wild-type AsCas12a. In some embodiments, the napDNAbp domain of any of the disclosed base editors comprises a mutant AsCas12a, such as an engineered AsCas12a, or enAsCas12a. In some embodiments, the napDNAbp domain of any of the disclosed based editors is comprises at least 80%, at least 85%, at least 90%, at least 95%, or at least 99% sequence identity to SEQ ID NO: 383. In some embodiments, the napDNAbp domain of any of the disclosed base editors comprises the amino acid sequence of SEQ ID NO: 383.

| Description | Sequence | SEQ ID NO: |
|---|---|---|
| SaCas9 | MGKRNYILGLDIGITSVGYGIIDYETRDVIDAGVRLFKEANVENNEGRRSKRGARRLKR | SEQ ID NO: |
| Staphylococcus | RRRHRIQRVKKLLFDYNLLTDHSELSGINPYEARVKGLSQKLSEEEFSAALLHLAKRRG | 377 |
| aureus | VHNVNEVEEDTGNELSTKEQISRNSKALEEKYVAELQLERLKKDGEVRGSINRFKTSDY | |
| 1053 AA | VKEAKQLLKVQKAYHQLDQSFIDTYIDLLETRRTYYEGPGEGSPFGWKDIKEWYEMLMG | |
| 123 kDa | HCTYFPEELRSVKYAYNADLYNALNDLNNLVITRDENEKLEYYEKFQIIENVFKQKKKP | |
| | TLKQIAKEILVNEEDIKGYRVTSTGKPEFTNLKVYHDIKDITARKEIIENAELLDQIAK | |
| | ILTIYQSSEDIQEELTNLNSELTQEEIEQISNLKGYTGTHNLSLKAINLILDELWHTND | |
| | NQIAIFNRLKLVPKKVDLSQQKEIPTTLVDDFILSPVVKRSFIQSIKVINAIIKKYGLP | |
| | NDIIIELAREKNSKDAQKMINEMQKRNRQTNERIEEIIRTTGKENAKYLIEKIKLHDMQ | |
| | EGKCLYSLEAIPLEDLLNNPFNYEVDHIIPRSVSFDNSFNNKVLVKQEENSKKGNRTPF | |
| | QYLSSSDSKISYETFKKHILNLAKGKGRISKTKKEYLLEERDINRFSVQKDFINRNLVD | |
| | TRYATRGLMNLLRSYFRVNNLDVKVKSINGGFTSFLRRKWKFKKERNKGYKHHAEDALI | |
| | IANADFIFKEWKKLDKAKKVMENQMFEEKQAESMPEIETEQEYKEIFITPHQIKHIKDF | |
| | KDYKYSHRVDKKPNRKLINDTLYSTRKDDKGNTLIVNNLNGLYDKDNDKLKKLINKSPE | |
| | KLLMYHHDPQTYQKLKLIMEQYGDEKNPLYKYYEETGNYLTKYSKKDNGPVIKKIKYYG | |
| | NKLNAHLDITDDYPNSRNKVVKLSLKPYRFDVYLDNGVYKFVTVKNLDVIKKENYYEVN | |
| | SKCYEEAKKLKKISNQAEFIASFYKNDLIKINGELYRVIGVNNDLLNRIEVNMIDITYR | |
| | EYLENMNDKRPPHIIKTIASKTQSIKKYSTDILGNLYEVKSKKHPQIIKK | |
| NmeCas9 | MAAFKPNSINYILGLDIGIASVGWAMVEIDEEENPIRLIDLGVRVFERAEVPKTGDSLA | SEQ ID NO: |
| N. | MARRLARSVRRLTRRRAHRLLRTRRLLKREGVLQAANFDENGLIKSLPNTPWQLRAAAL | 378 |
| meningitidis | DRKLTPLEWSAVLLHLIKHRGYLSQRKNEGETADKELGALLKGVAGNAHALQTGDFRTP | |
| 1083 AA | AELALNKFEKESGHIRNQRSDYSHTFSRKDLQAELILLFEKQKEFGNPHVSGGLKEGIE | |
| 124.5 kDa | TLLMTQRPALSGDAVQKMLGHCTFEPAEPKAAKNTYTAERFIWLTKLNNLRILEQGSER | |
| | PLTDTERATLMDEPYRKSKLTYAQARKLLGLEDTAFFKGLRYGKDNAEASTLMEMKAYH | |
| | AISRALEKEGLKDKKSPLNLSPELQDEIGTAFSLFKTDEDITGRLKDRIQPEILEALLK | |
| | HISFDKFVQISLKALRRIVPLMEQGKRYDEACAEIYGDHYGKKNTEEKIYLPPIPADEI | |
| | RNPVVLRALSQARKVINGVVRRYGSPARIHIETAREVGKSFKDRKEIEKRQEENRKDRE | |
| | KAAAKFREYFPNFVGEPKSKDILKLRLYEQQHGKCLYSGKEINLGRLNEKGYVEIDAAL | |
| | PFSRTWDDSFNNKVLVLGSENQNKGNQTPYEYFNGKDNSREWQEFKARVETSRFPRSKK | |
| | QRILLQKFDEDGFKERNLNDTRYVNRFLCQFVADRMRLTGKGKKRVFASNGQITNLLRG | |
| | FWGLRKVRAENDRHHALDAVVVACSTVAMQQKITRFVRYKEMNAFDGKTIDKETGEVLH | |
| | QKTHFPQPWEFFAQEVMIRVFGKPDGKPEFEEADTLEKLRTLLAEKLSSRPEAVHEYVT | |
| | PLFVSRAPNRKMSGQGHMETVKSAKRLDEGVSVLRVPLTQLKLKDLEKMVNREREPKLY | |
| | EALKARLEAHKDDPAKAFAEPFYKYDKAGNRTQQVKAVRVEQVQKTGVWVRNHNGIADN | |
| | ATMVRVDVFEKGDKYYLVPIYSWQVAKGILPDRAVVQGKDEEDWQLIDDSFNFKFSLHP | |
| | NDLVEVITKKARMFGYFASCHRGTGNINIRIHDLDHKIGKNGILEGIGVKTALSFQKYQ | |
| | IDELGKEIRPCRLKKRPPVR | |
| CjCas9 | MARILAFDIGISSIGWAFSENDELKDCGVRIFTKVENPKTGESLALPRRLARSARKRLA | SEQ ID NO: |
| C. jejuni | RRKARLNHLKHLIANEFKLNYEDYQSFDESLAKAYKGSLISPYELRFRALNELLSKQDF | 379 |
| 984 AA | ARVILHIAKRRGYDDIKNSDDKEKGAILKAIKQNEEKLANYQSVGEYLYKEYFQKFKEN | |
| 114.9 kDa | SKEFTNVRNKKESYERCIAQSFLKDELKLIFKKQREFGFSFSKKFEEEVLSVAFYKRAL | |
| | KDFSHLVGNCSFFTDEKRAPKNSPLAFMFVALTRIINLLNNLKNTEGILYTKDDLNALL | |
| | NEVLKNGTLTYKQTKKLLGLSDDYEFKGEKGTYFIEFKKYKEFIKALGEHNLSQDDLNE | |
| | IAKDITLIKDEIKLKKALAKYDLNQNQIDSLSKLEFKDHLNISFKALKLVTPLMLEGKK | |
| | YDEACNELNLKVAINEDKKDFLPAFNETYYKDEVTNPVVLRAIKEYRKVLNALLKKYGK | |
| | VHKINIELAREVGKNHSQRAKIEKEQNENYKAKKDAELECEKLGLKINSKNILKLRLFK | |
| | EQKEFCAYSGEKIKISDLQDEKMLEIDHIYPYSRSFDDSYMNKVLVFTKQNQEKLNQTP | |
| | FEAFGNDSAKWQKIEVLAKNLPTKKQKRILDKNYKDKEQKNFKDRNLNDTRYIARLVLN | |
| | YTKDYLDFLPLSDDENTKLNDTQKGSKVHVEAKSGMLTSALRHTWGFSAKDRNNHLHHA | |
| | IDAVIIAYANNSIVKAFSDFKKEQESNSAELYAKKISELDYKNKRKFFEPFSGFRQKVL | |
| | DKIDEIFVSKPERKKPSGALHEETFRKEEEFYQSYGGKEGVLKALELGKIRKVNGKIVK | |
| | NGDMFRVDIFKHKKTNKFYAVPIYTMDFALKVLPNKAVARSKKGEIKDWILMDENYEFC | |
| | FSLYKDSLILIQTKDMQEPEFVYYNAFTSSTVSLIVSKHDNKFETLSKNQKILFKNANE | |
| | KEVIAKSIGIQNLKVFEKYIVSALGEVTKAEFRQREDFKK | |
| GeoCas 9 | MRYKIGLDIGITSVGWAVMNLDIPRIEDLGVRIFDRAENPQTGESLALPRRLARSARRR | SEQ ID NO: |
| G. | LRRRKHRLERIRRLVIREGILTKEELDKLFEEKHEIDVWQLRVEALDRKLNNDELARVL | 380 |
| stearothermo- | LHLAKRRGFKSNRKSERSNKENSTMLKHIEENRAILSSYRTVGEMIVKDPKFALHKRNK | |
| philus | GENYTNTIARDDLEREIRLIFSKQREFGNMSCTEEFENEYITIWASQRPVASKDDIEKK | |
| 1087 AA | VGFCTFEPKEKRAPKATYTFQSFIAWEHINKLRLISPSGARGLTDEERRLLYEQAFQKN | |
| 127 kDa | KITYHDIRTLLHLPDDTYFKGIVYDRGESRKQNENIRFLELDAYHQIRKAVDKVYGKGK | |
| | SSSFLPIDFDTFGYALTLFKDDADIHSYLRNEYEQNGKRMPNLANKVYDNELIEELLNL | |
| | SFTKFGHLSLKALRSILPYMEQGEVYSSACERAGYTFTGPKKKQKTMLLPNIPPIANPV | |
| | VMRALTQARKVVNAIIKKYGSPVSIHIELARDLSQTFDERRKTKKEQDENRKKNETAIR | |
| | QLMEYGLTLNPTGHDIVKFKLWSEQNGRCAYSLQPIEIERLLEPGYVEVDHVIPYSRSL | |
| | DDSYTNKVLVLTRENREKGNRIPAEYLGVGTERWQQFETFVLTNKQFSKKKRDRLLRLH | |
| | YDENEETEFKNRNLNDTRYISRFFANFIREHLKFAESDDKQKVYTVNGRVTAHLRSRWE | |
| | FNKNREESDLHHAVDAVIVACTTPSDIAKVTAFYQRREQNKELAKKTEPHFPQPWPHFA | |
| | DELRARLSKHPKESIKALNLGNYDDQKLESLQPVFVSRMPKRSVTGAAHQETLRRYVGI | |
| | DERSGKIQTVVKTKLSEIKLDASGHFPMYGKESDPRTYEAIRQRLLEHNNDPKKAFQEP | |
| | LYKPKKNGEPGPVIRTVKIIDTKNQVIPLNDGKTVAYNSNIVRVDVFEKDGKYYCVPVY | |
| | TMDIMKGILPNKAIEPNKPYSEWKEMTEDYTFRFSLYPNDLIRIELPREKTVKTAAGEE | |
| | INVKDVFVYYKTIDSANGGLELISHDHRFSLRGVGSRTLKRFEKYQVDVLGNIYKVRGE | |
| | KRVGLASSAHSKPGKTIRPLQSTRD | |
| LbCas12a | MSKLEKFTNCYSLSKTLRFKAIPVGKTQENIDNKRLLVEDEKRAEDYKGVKKLLDRYYL | SEQ ID NO: |
| L. bacterium | SFINDVLHSIKLKNLNNYISLFRKKTRTEKENKELENLEINLRKEIAKAFKGNEGYKSL | 381 |
| 1228 AA | FKKDIIETILPEFLDDKDEIALVNSFNGFTTAFTGFFDNRENMFSEEAKSTSIAFRCIN | |
| 143.9 kDa | ENLTRYISNMDIFEKVDAIFDKHEVQEIKEKILNSDYDVEDFFEGEFFNFVLTQEGIDV | |
| | YNAIIGGFVTESGEKIKGLNEYINLYNQKTKQKLPKFKPLYKQVLSDRESLSFYGEGYT | |
| | SDEEVLEVFRNTLNKNSEIFSSIKKLEKLFKNFDEYSSAGIFVKNGPAISTISKDIFGE | |
| | WNVIRDKWNAEYDDIHLKKKAVVTEKYEDDRRKSFKKIGSFSLEQLQEYADADLSVVEK | |
| | LKEIIIQKVDEIYKVYGSSEKLFDADFVLEKSLKKNDAVVAIMKDLLDSVKSFENYIKA | |
| | FFGEGKETNRDESFYGDFVLAYDILLKVDHIYDAIRNYVTQKPYSKDKFKLYFQNPQFM | |
| | GGWDKDKETDYRATILRYGSKYYLAIMDKKYAKCLQKIDKDDVNGNYEKINYKLLPGPN | |
| | KMLPKVFFSKKWMAYYNPSEDIQKIYKNGTFKKGDMFNLNDCHKLIDFFKDSISRYPKW | |
| | SNAYDFNFSETEKYKDIAGFYREVEEQGYKVSFESASKKEVDKLVEEGKLYMFQIYNKD | |
| | FSDKSHGTPNLHTMYFKLLFDENNHGQIRLSGGAELFMRRASLKKEELVVHPANSPIAN | |
| | KNPDNPKKTTTLSYDVYKDKRFSEDQYELHIPIAINKCPKNIFKINTEVRVLLKHDDNP | |
| | YVIGIDRGERNLLYIVVVDGKGNIVEQYSLNEIINNFNGIRIKTDYHSLLDKKEKERFE | |
| | ARQNWTSIENIKELKAGYISQVVHKICELVEKYDAVIALEDLNSGFKNSRVKVEKQVYQ | |
| | KFEKMLIDKLNYMVDKKSNPCATGGALKGYQITNKFESFKSMSTQNGFIFYIPAWLTSK | |
| | IDPSTGFVNLLKTKYTSIADSKKFISSFDRIMYVPEEDLFEFALDYKNFSRTDADYIKK | |
| | WKLYSYGNRIRIFRNPKKNNVFDWEEVCLTSAYKELFNKYGINYQQGDIRALLCEQSDK | |
| | AFYSSFMALMSLMLQMRNSITGRTDVDFLISPVKNSDGIFYDSRNYEAQENAILPKNAD | |
| | ANGAYNIARKVLWAIGQFKKAEDEKLDKVKIAISNKEWLEYAQTSVKH | |
| BhCas12b | MATRSFILKIEPNEEVKKGLWKTHEVLNHGIAYYMNILKLIRQEAIYEHHEQDPKNPKK | SEQ ID NO: |
| B. hisashii | VSKAEIQAELWDFVLKMQKCNSFTHEVDKDEVFNILRELYEELVPSSVEKKGEANQLSN | 382 |
| 1108 AA | KFLYPLVDPNSQSGKGTASSGRKPRWYNLKIAGDPSWEEEKKKWEEDKKKDPLAKILGK | |
| 130.4kDa | LAEYGLIPLFIPYTDSNEPIVKEIKWMEKSRNQSVRRLDKDMFIQALERFLSWESWNLK | |
| | VKEEYEKVEKEYKTLEERIKEDIQALKALEQYEKERQEQLLRDTLNTNEYRLSKRGLRG | |
| | WREIIQKWLKMDENEPSEKYLEVFKDYQRKHPREAGDYSVYEFLSKKENHFIWRNHPEY | |
| | PYLYATFCEIDKKKKDAKQQATFTLADPINHPLWVRFEERSGSNLNKYRILTEQLHTEK | |
| | LKKKLTVQLDRLIYPTESGGWEEKGKVDIVLLPSRQFYNQIFLDIEEKGKHAFTYKDES | |
| | IKFPLKGTLGGARVQFDRDHLRRYPHKVESGNVGRIYFNMTVNIEPTESPVSKSLKIHR | |
| | DDFPKVVNFKPKELTEWIKDSKGKKLKSGIESLEIGLRVMSIDLGQRQAAAASIFEVVD | |
| | QKPDIEGKLFFPIKGTELYAVHRASFNIKLPGETLVKSREVLRKAREDNLKLMNQKLNF | |
| | LRNVLHFQQFEDITEREKRVTKWISRQENSDVPLVYQDELIQIRELMYKPYKDWVAFLK | |
| | QLHKRLEVEIGKEVKHWRKSLSDGRKGLYGISLKNIDEIDRTRKFLLRWSLRPTEPGEV | |
| | RRLEPGQRFAIDQLNHLNALKEDRLKKMANTIIMHALGYCYDVRKKKWQAKNPACQIIL | |
| | FEDLSNYNPYEERSRFENSKLMKWSRREIPRQVALQGEIYGLQVGEVGAQFSSRFHAKT | |
| | GSPGIRCSVVTKEKLQDNRFFKNLQREGRLTLDKIAVLKEGDLYPDKGGEKFISLSKDR | |
| | KCVTTHADINAAQNLQKRFWTRTHGFYKVYCKAYQVDGQTVYIPESKDQKQKIIEEFGE | |
| | GYFILKDGVYEWVNAGKLKIKKGSSKQSSSELVDSDILKDSFDLASELKGEKLMLYRDP | |
| | SGNVFPSDKWMAAGVFFGKLERILISKLTNQYSISTIEDDSSKQSM | |

Additional exemplary Cas9 equivalent protein sequences can include the following:

| Description | Sequence | |
|---|---|---|
| AsCas12a | MTQFEGFTNLYQVSKTLRFELIPQGKTLKHIQEQGFIEEDKARNDHYKELKPIIDRIYKT | |
| (previously | YADQCLQLVQLDWENLSAAIDSYRKEKTEETRNALIEEQATYRNAIHDYFIGRTDNLTDA | |
| known as | INKRHAEIYKGLFKAELFNGKVLKQLGTVTTTEHENALLRSFDKFTTYFSGFYENRKNVF | |
| Cpf1) | SAEDISTAIPHRIVQDNFPKFKENCHIFTRLITAVPSLREHFENVKKAIGIFVSTSIEEV | |
| Acidaminococcus | FSFPFYNQLLTQTQIDLYNQLLGGISREAGTEKIKGLNEVLNLAIQKNDETAHIIASLPH | |
| sp. | RFIPLFKQILSDRNTLSFILEEFKSDEEVIQSFCKYKTLLRNENVLETAEALFNELNSID | |
| (strain | LTHIFISHKKLETISSALCDHWDTLRNALYERRISELTGKITKSAKEKVQRSLKHEDINL | |
| BV3L6) | QEIISAAGKELSEAFKQKTSEILSHAHAALDQPLPTTLKKQEEKEILKSQLDSLLGLYHL | |
| UniProtKB | LDWFAVDESNEVDPEFSARLTGIKLEMEPSLSFYNKARNYATKKPYSVEKFKLNFQMPTL | |
| U2UMQ6 | ASGWDVNKEKNNGAILFVKNGLYYLGIMPKQKGRYKALSFEPTEKTSEGFDKMYYDYFPD | |
| | AAKMIPKCSTQLKAVTAHFQTHTTPILLSNNFIEPLEITKEIYDLNNPEKEPKKFQTAYA | |
| | KKTGDQKGYREALCKWIDFTRDFLSKYTKTTSIDLSSLRPSSQYKDLGEYYAELNPLLYH | |
| | ISFQRIAEKEIMDAVETGKLYLFQIYNKDFAKGHHGKPNLHTLYWTGLFSPENLAKTSIK | |
| | LNGQAELFYRPKSRMKRMAHRLGEKMLNKKLKDQKTPIPDTLYQELYDYVNHRLSHDLSD | |
| | EARALLPNVITKEVSHEIIKDRRFTSDKFFFHVPITLNYQAANSPSKFNQRVNAYLKEHP | |
| | ETPIIGIDRGERNLIYITVIDSTGKILEQRSLNTIQQFDYQKKLDNREKERVAARQAWSV | |
| | VGTIKDLKQGYLSQVIHEIVDLMIHYQAVVVLENLNFGFKSKRTGIAEKAVYQQFEKMLI | |
| | DKLNCLVLKDYPAEKVGGVLNPYQLTDQFTSFAKMGTQSGFLFYVPAPYTSKIDPLTGFV | |
| | DPFVWKTIKNHESRKHFLEGFDFLHYDVKTGDFILHFKMNRNLSFQRGLPGFMPAWDIVF | |
| | EKNETQFDAKGTPFIAGKRIVPVIENHRFTGRYRDLYPANELIALLEEKGIVFRDGSNIL | |
| | PKLLENDDSHAIDTMVALIRSVLQMRNSNAATGEDYINSPVRDLNGVCFDSRFQNPEWPM | |
| | DADANGAYHIALKGQLLLNHLKESKDLKLQNGISNQDWLAYIQELRN (SEQ ID NO: 383) | |
| AsCas12a | MTQFEGFTNLYQVSKTLRFELIPQGKTLKHIQEQGFIEEDKARNDHYKELKPIIDRIYKT | |
| nickase | YADQCLQLVQLDWENLSAAIDSYRKEKTEETRNALIEEQATYRNAIHDYFIGRTDNLTDA | |
| (e.g., | INKRHAEIYKGLFKAELFNGKVLKQLGTVTTTEHENALLRSFDKFTTYFSGFYENRKNVF | |
| R1226A) | SAEDISTAIPHRIVQDNFPKFKENCHIFTRLITAVPSLREHFENVKKAIGIFVSTSIEEV | |
| | FSFPFYNQLLTQTQIDLYNQLLGGISREAGTEKIKGLNEVLNLAIQKNDETAHIIASLPH | |
| | RFIPLFKQILSDRNTLSFILEEFKSDEEVIQSFCKYKTLLRNENVLETAEALFNELNSID | |
| | LTHIFISHKKLETISSALCDHWDTLRNALYERRISELTGKITKSAKEKVQRSLKHEDINL | |
| | QEIISAAGKELSEAFKQKTSEILSHAHAALDQPLPTTLKKQEEKEILKSQLDSLLGLYHL | |
| | LDWFAVDESNEVDPEFSARLTGIKLEMEPSLSFYNKARNYATKKPYSVEKFKLNFQMPTL | |
| | ASGWDVNKEKNNGAILFVKNGLYYLGIMPKQKGRYKALSFEPTEKTSEGFDKMYYDYFPD | |
| | AAKMIPKCSTQLKAVTAHFQTHTTPILLSNNFIEPLEITKEIYDLNNPEKEPKKFQTAYA | |
| | KKTGDQKGYREALCKWIDFTRDFLSKYTKTTSIDLSSLRPSSQYKDLGEYYAELNPLLYH | |
| | ISFQRIAEKEIMDAVETGKLYLFQIYNKDFAKGHHGKPNLHTLYWTGLFSPENLAKTSIK | |
| | LNGQAELFYRPKSRMKRMAHRLGEKMLNKKLKDQKTPIPDTLYQELYDYVNHRLSHDLSD | |
| | EARALLPNVITKEVSHEIIKDRRFTSDKFFFHVPITLNYQAANSPSKFNQRVNAYLKEHP | |
| | ETPIIGIDRGERNLIYITVIDSTGKILEQRSLNTIQQFDYQKKLDNREKERVAARQAWSV | |
| | VGTIKDLKQGYLSQVIHEIVDLMIHYQAVVVLENLNFGFKSKRTGIAEKAVYQQFEKMLI | |
| | DKLNCLVLKDYPAEKVGGVLNPYQLTDQFTSFAKMGTQSGFLFYVPAPYTSKIDPLTGFV | |
| | DPFVWKTIKNHESRKHFLEGFDFLHYDVKTGDFILHFKMNRNLSFQRGLPGFMPAWDIVE | |
| | EKNETQFDAKGTPFIAGKRIVPVIENHRFTGRYRDLYPANELIALLEEKGIVFRDGSNIL | |
| | PKLLENDDSHAIDTMVALIRSVLQMANSNAATGEDYINSPVRDLNGVCFDSRFQNPEWPM | |
| | DADANGAYHIALKGQLLLNHLKESKDLKLQNGISNQDWLAYIQELRN (SEQ ID NO: 384) | |
| LbCas12a | 1 | MNYKTGLEDF IGKESLSKTL RNALIPTEST KIHMEEMGVI RDDELRAEKQ QELKEIMDDY |
| (previously | 61 | YRTFIEEKLG QIQGIQWNSL FQKMEETMED ISVRKDLDKI QNEKRKEICC YFTSDKRFKD |
| known as | 121 | LFNAKLITDI LPNFIKDNKE YTEEEKAEKE QTRVLFQRFA TAFTNYFNQR RNNFSEDNIS |
| Cpf1) | 181 | TAISFRIVNE NSEIHLQNMR AFQRIEQQYP EEVCGMEEEY KDMLQEWQMK HIYSVDFYDR |
| Lachnospiraceae | 241 | ELTQPGIEYY NGICGKINEH MNQFCQKNRI NKNDFRMKKL HKQILCKKSS YYEIPFRFES |
| bacterium | 301 | DQEVYDALNE FIKTMKKKEI IRRCVHLGQE CDDYDLGKIY ISSNKYEQIS NALYGSWDTI |
| GAM79 | 361 | RKCIKEEYMD ALPGKGEKKE EKAEAAAKKE EYRSIADIDK IISLYGSEMD RTISAKKCIT |
| Ref Seq. | 421 | EICDMAGQIS IDPLVCNSDI KLLQNKEKTT EIKTILDSFL HVYQWGQTFI VSDIIEKDSY |
| WP_119623382. | 481 | FYSELEDVLE DFEGITTLYN HVRSYVTQKP YSTVKFKLHF GSPTLANGWS QSKEYDNNAI |
| 1 | 541 | LLMRDQKFYL GIFNVRNKPD KQIIKGHEKE EKGDYKKMIY NLLPGPSKML PKVFITSRSG |
| | 601 | QETYKPSKHI LDGYNEKRHI KSSPKFDLGY CWDLIDYYKE CIHKHPDWKN YDFHFSDTKD |
| | 661 | YEDISGFYRE VEMQGYQIKW TYISADEIQK LDEKGQIFLF QIYNKDFSVH STGKDNLHTM |
| | 721 | YLKNLFSEEN LKDIVLKLNG EAELFFRKAS IKTPIVHKKG SVLVNRSYTQ TVGNKEIRVS |
| | 781 | IPEEYYTEIY NYLNHIGKGK LSSEAQRYLD EGKIKSFTAT KDIVKNYRYC CDHYFLHLPI |
| | 841 | TINFKAKSDV AVNERTLAYI AKKEDIHIIG IDRGERNLLY ISVVDVHGNI REQRSFNIVN |
| | 901 | GYDYQQKLKD REKSRDAARK NWEEIEKIKE LKEGYLSMVI HYIAQLVVKY NAVVAMEDLN |
| | 961 | YGFKTGRFKV ERQVYQKFET MLIEKLHYLV FKDREVCEEG GVLRGYQLTY IPESLKKVGK |
| | 1021 | QCGFIFYVPA GYTSKIDPTT GFVNLFSFKN LTNRESRQDF VGKFDEIRYD RDKKMFEFSF |
| | 1081 | DYNNYIKKGT ILASTKWKVY TNGTRLKRIV VNGKYTSQSM EVELTDAMEK MLQRAGIEYH |
| | 1141 | DGKDLKGQIV EKGIEAEIID IFRLTVQMRN SRSESEDREY DRLISPVLND KGEFFDTATA |
| | 1201 | DKTLPQDADA NGAYCIALKG LYEVKQIKEN WKENEQFPRN KLVQDNKTWF DFMQKKRYL |
| | | (SEQ ID NO: 385) |
| PcCas12a - | 1 | MAKNFEDFKR LYSLSKTLRF EAKPIGATLD NIVKSGLLDE DEHRAASYVK VKKLIDEYHK |
| previously | 61 | VFIDRVLDDG CLPLENKGNN NSLAEYYESY VSRAQDEDAK KKFKEIQQNL RSVIAKKLTE |
| known at Cpf1 | 121 | DKAYANLFGN KLIESYKDKE DKKKIIDSDL IQFINTAEST QLDSMSQDEA KELVKEFWGF |
| Prevotella | 181 | VTYFYGFFDN RKNMYTAEEK STGIAYRLVN ENLPKFIDNI EAFNRAITRP EIQENMGVLY |
| copri | 241 | SDFSEYLNVE SIQEMFQLDY YNMLLTQKQI DVYNAIIGGK TDDEHDVKIK GINEYINLYN |
| Ref Seq. | 301 | QQHKDDKLPK LKALFKQILS DRNAISWLPE EFNSDQEVLN AIKDCYERLA ENVLGDKVLK |
| WP_119227726. | 361 | SLLGSLADYS LDGIFIRNDL QLTDISQKMF GNWGVIQNAI MQNIKRVAPA RKHKESEEDY |
| 1 | 421 | EKRIAGIFKK ADSFSISYIN DCLNEADPNN AYFVENYFAT FGAVNTPTMQ RENLFALVQN |
| | 481 | AYTEVAALLH SDYPTVKHLA QDKANVSKIK ALLDAIKSLQ HFVKPLLGKG DESDKDERFY |
| | 541 | GELASLWAEL DTVTPLYNMI RNYMTRKPYS QKKIKLNFEN PQLLGGWDAN KEKDYATIIL |
| | 601 | RRNGLYYLAI MDKDSRKLLG KAMPSDGECY EKMVYKFFKD VTTMIPKCST QLKDVQAYFK |
| | 661 | VNTDDYVLNS KAFNKPLTIT KEVFDLNNVL YGKYKKFQKG YLTATGDNVG YTHAVNVWIK |
| | 721 | FCMDFLNSYD STCIYDFSSL KPESYLSLDA FYQDANLLLY KLSFARASVS YINQLVEEGK |
| | 781 | MYLFQIYNKD FSEYSKGTPN MHTLYWKALF DERNLADVVY KLNGQAEMFY RKKSIENTHP |
| | 841 | THPANHPILN KNKDNKKKES LFDYDLIKDR RYTVDKFMFH VPITMNFKSV GSENINQDVK |
| | 901 | AYLRHADDMH IIGIDRGERH LLYLVVIDLQ GNIKEQYSLN EIVNEYNGNT YHTNYHDLLD |
| | 961 | VREEERLKAR QSWQTIENIK ELKEGYLSQV IHKITQLMVR YHAIVVLEDL SKGFMRSRQK |
| | 1021 | VEKQVYQKFE KMLIDKLNYL VDKKTDVSTP GGLLNAYQLT CKSDSSQKLG KQSGFLFYIP |
| | 1081 | AWNTSKIDPV TGFVNLLDTH SLNSKEKIKA FFSKFDAIRY NKDKKWFEFN LDYDKFGKKA |
| | 1141 | EDTRTKWTLC TRGMRIDTFR NKEKNSQWDN QEVDLTTEMK SLLEHYYIDI HGNLKDAISA |
| | 1201 | QTDKAFFTGL LHILKLTLQM RNSITGTETD YLVSPVADEN GIFYDSRSCG NQLPENADAN |
| | 1261 | GAYNIARKGL MLIEQIKNAE DLNNVKFDIS NKAWLNFAQQ KPYKNG |
| | | (SEQ ID NO: 386) |
| ErCas12a - | 1 | MFSAKLISDI LPEFVIHNNN YSASEKEEKT QVIKLFSRFA TSFKDYFKNR ANCFSANDIS |
| previously | 61 | SSSCHRIVND NAEIFFSNAL VYRRIVKNLS NDDINKISGD MKDSLKEMSL EEIYSYEKYG |
| known at Cpf1 | 121 | EFITQEGISF YNDICGKVNL FMNLYCQKNK ENKNLYKLRK LHKQILCIAD TSYEVPYKFE |
| Eubacterium | 181 | SDEEVYQSVN GFLDNISSKH IVERLRKIGE NYNGYNLDKI YIVSKFYESV SQKTYRDWET |
| rectale | 241 | INTALEIHYN NILPGNGKSK ADKVKKAVKN DLQKSITEIN ELVSNYKLCP DDNIKAETYI |
| Ref Seq. | 301 | HEISHILNNF EAQELKYNPE IHLVESELKA SELKNVLDVI MNAFHWCSVF MTEELVDKDN |
| WP119223642. | 361 | NFYAELEEIY DEIYPVISLY NLVRNYVTQK PYSTKKIKLN FGIPTLADGW SKSKEYSNNA |
| 1 | 421 | IILMRDNLYY LGIFNAKNKP DKKIIEGNTS ENKGDYKKMI YNLLPGPNKM IPKVFLSSKT |
| | 481 | GVETYKPSAY ILEGYKQNKH LKSSKDFDIT FCHDLIDYFK NCIAIHPEWK NFGFDFSDTS |
| | 541 | TYEDISGFYR EVELQGYKID WTYISEKDID LLQEKGQLYL FQIYNKDFSK KSSGNDNLHT |
| | 601 | MYLKNLFSEE NLKDIVLKLN GEAEIFFRKS SIKNPIIHKK GSILVNRTYE AEEKDQFGNI |
| | 661 | QIVRKTIPEN IYQELYKYFN DKSDKELSDE AAKLKNVVGH HEAATNIVKD YRYTYDKYFL |
| | 721 | HMPITINFKA NKTSFINDRI LQYIAKEKDL HVIGIDRGER NLIYVSVIDT CGNIVEQKSF |
| | 781 | NIVNGYDYQI KLKQQEGARQ IARKEWKEIG KIKEIKEGYL SLVIHEISKM VIKYNAIIAM |
| | 841 | EDLSYGFKKG RFKVERQVYQ KFETMLINKL NYLVFKDISI TENGGLLKGY QLTYIPDKLK |
| | 901 | NVGHQCGCIF YVPAAYTSKI DPTTGFVNIF KFKDLTVDAK REFIKKFDSI RYDSDKNLFC |
| | 961 | FTFDYNNFIT QNTVMSKSSW SVYTYGVRIK RRFVNGRFSN ESDTIDITKD MEKTLEMTDI |
| | 1021 | NWRDGHDLRQ DIIDYEIVQH IFEIFKLTVQ MRNSLSELED RDYDRLISPV LNENNIFYDS |
| | 1081 | AKAGDALPKD ADANGAYCIA LKGLYEIKQI TENWKEDGKF SRDKLKISNK DWFDFIQNKR |
| | 1141 | YL (SEQ ID NO: 387) |
| CsCas12a - | 1 | MNYKTGLEDF IGKESLSKTL RNALIPTEST KIHMEEMGVI RDDELRAEKQ QELKEIMDDY |
| previously | 61 | YRAFIEEKLG QIQGIQWNSL FQKMEETMED ISVRKDLDKI QNEKRKEICC YFTSDKRFKD |
| known at Cpf1 | 121 | LFNAKLITDI LPNFIKDNKE YTEEEKAEKE QTRVLFQRFA TAFTNYFNQR RNNFSEDNIS |
| Clostridium | 181 | TAISFRIVNE NSEIHLQNMR AFQRIEQQYP EEVCGMEEEY KDMLQEWQMK HIYLVDFYDR |
| sp. AF34-10BH | 241 | VLTQPGIEYY NGICGKINEH MNQFCQKNRI NKNDFRMKKL HKQILCKKSS YYEIPFRFES |
| Ref Seq. | 301 | DQEVYDALNE FIKTMKEKEI ICRCVHLGQK CDDYDLGKIY ISSNKYEQIS NALYGSWDTI |
| WP_118538418. | 361 | RKCIKEEYMD ALPGKGEKKE EKAEAAAKKE EYRSIADIDK IISLYGSEMD RTISAKKCIT |
| 1 | 421 | EICDMAGQIS TDPLVCNSDI KLLQNKEKTT EIKTILDSFL HVYQWGQTFI VSDIIEKDSY |
| | 481 | FYSELEDVLE DFEGITTLYN HVRSYVTQKP YSTVKFKLHF GSPTLANGWS QSKEYDNNAI |
| | 541 | LLMRDQKFYL GIFNVRNKPD KQIIKGHEKE EKGDYKKMIY NLLPGPSKML PKVFITSRSG |
| | 601 | QETYKPSKHI LDGYNEKRHI KSSPKFDLGY CWDLIDYYKE CIHKHPDWKN YDFHFSDTKD |
| | 661 | YEDISGFYRE VEMQGYQIKW TYISADEIQK LDEKGQIFLF QIYNKDFSVH STGKDNLHTM |
| | 721 | YLKNLFSEEN LKDIVLKLNG EAELFFRKAS IKTPVVHKKG SVLVNRSYTQ TVGDKEIRVS |
| | 781 | IPEEYYTEIY NYLNHIGRGK LSTEAQRYLE ERKIKSFTAT KDIVKNYRYC CDHYFLHLPI |
| | 841 | TINFKAKSDI AVNERTLAYI AKKEDIHIIG IDRGERNLLY ISVVDVHGNI REQRSFNIVN |
| | 901 | GYDYQQKLKD REKSRDAARK NWEEIEKIKE LKEGYLSMVI HYIAQLVVKY NAVVAMEDLN |
| | 961 | YGFKTGRFKV ERQVYQKFET MLIEKLHYLV FKDREVCEEG GVLRGYQLTY IPESLKKVGK |
| | 1021 | QCGFIFYVPA GYTSKIDPTT GFVNLFSFKN LTNRESRQDF VGKFDEIRYD RDKKMFEFSF |
| | 1081 | DYNNYIKKGT MLASTKWKVY TNGTRLKRIV VNGKYTSQSM EVELTDAMEK MLQRAGIEYH |
| | 1141 | DGKDLKGQIV EKGIEAEIID IFRLTVQMRN SRSESEDREY DRLISPVLND KGEFFDTATA |
| | 1201 | DKTLPQDADA NGAYCIALKG LYEVKQIKEN WKENEQFPRN KLVQDNKTWF DFMQKKRYL |
| | | (SEQ ID NO: 388) |
| BhCas12b | 1 | MATRSFILKI EPNEEVKKGL WKTHEVLNHG IAYYMNILKL IRQEAIYEHH EQDPKNPKKV |
| Bacillus | 61 | SKAEIQAELW DFVLKMQKCN SFTHEVDKDE VFNILRELYE ELVPSSVEKK GEANQLSNKF |
| hisashii | 121 | LYPLVDPNSQ SGKGTASSGR KPRWYNLKIA GDPSWEEEKK KWEEDKKKDP LAKILGKLAE |
| Ref Seq. | 181 | YGLIPLFIPY TDSNEPIVKE IKWMEKSRNQ SVRRLDKDMF IQALERFLSW ESWNLKVKEE |
| WP_095142515. | 241 | YEKVEKEYKT LEERIKEDIQ ALKALEQYEK ERQEQLLRDT LNTNEYRLSK RGLRGWREII |
| 1 | 301 | QKWLKMDENE PSEKYLEVFK DYQRKHPREA GDYSVYEFLS KKENHFIWRN HPEYPYLYAT |
| | 361 | FCEIDKKKKD AKQQATFTLA DPINHPLWVR FEERSGSNLN KYRILTEQLH TEKLKKKLTV |
| | 421 | QLDRLIYPTE SGGWEEKGKV DIVLLPSRQF YNQIFLDIEE KGKHAFTYKD ESIKFPLKGT |
| | 481 | LGGARVQFDR DHLRRYPHKV ESGNVGRIYF NMTVNIEPTE SPVSKSLKIH RDDFPKVVNF |
| | 541 | KPKELTEWIK DSKGKKLKSG IESLEIGLRV MSIDLGQRQA AAASIFEVVD QKPDIEGKLF |
| | 601 | FPIKGTELYA VHRASFNIKL PGETLVKSRE VLRKAREDNL KLMNQKLNFL RNVLHFQQFE |
| | 661 | DITEREKRVT KWISRQENSD VPLVYQDELI QIRELMYKPY KDWVAFLKQL HKRLEVEIGK |
| | 721 | EVKHWRKSLS DGRKGLYGIS LKNIDEIDRT RKFLLRWSLR PTEPGEVRRL EPGQRFAIDQ |
| | 781 | LNHLNALKED RLKKMANTII MHALGYCYDV RKKKWQAKNP ACQIILFEDL SNYNPYEERS |
| | 841 | RFENSKLMKW SRREIPRQVA LQGEIYGLQV GEVGAQFSSR FHAKTGSPGI RCSVVTKEKL |
| | 901 | QDNRFFKNLQ REGRLTLDKI AVLKEGDLYP DKGGEKFISL SKDRKCVTTH ADINAAQNLQ |
| | 961 | KRFWTRTHGF YKVYCKAYQV DGQTVYIPES KDQKQKIIEE FGEGYFILKD GVYEWVNAGK |
| | 1021 | LKIKKGSSKQ SSSELVDSDI LKDSFDLASE LKGEKLMLYR DPSGNVFPSD KWMAAGVFFG |
| | 1081 | KLERILISKL TNQYSISTIE DDSSKQSM (SEQ ID NO: 389) |
| ThCas12b | 1 | MSEKTTQRAY TLRLNRASGE CAVCQNNSCD CWHDALWATH KAVNRGAKAF GDWLLTLRGG |
| Thermomonas | 61 | LCHTLVEMEV PAKGNNPPQR PTDQERRDRR VLLALSWLSV EDEHGAPKEF IVATGRDSAD |
| hydrothermalis | 121 | DRAKKVEEKL REILEKRDFQ EHEIDAWLQD CGPSLKAHIR EDAVWVNRRA LFDAAVERIK |
| Ref Seq. | 181 | TLTWEEAWDF LEPFFGTQYF AGIGDGKDKD DAEGPARQGE KAKDLVQKAG QWLSARFGIG |
| WP_072754838 | 241 | TGADFMSMAE AYEKIAKWAS QAQNGDNGKA TIEKLACALR PSEPPTLDTV LKCISGPGHK |
| | 301 | SATREYLKTL DKKSTVTQED LNQLRKLADE DARNCRKKVG KKGKKPWADE VLKDVENSCE |
| | 361 | LTYLQDNSPA RHREFSVMLD HAARRVSMAH SWIKKAEQRR RQFESDAQKL KNLQERAPSA |
| | 421 | VEWLDRFCES RSMTTGANTG SGYRIRKRAI EGWSYVVQAW AEASCDTEDK RIAAARKVQA |
| | 481 | DPEIEKFGDI QLFEALAADE AICVWRDQEG TQNPSILIDY VTGKTAEHNQ KRFKVPAYRH |
| | 541 | PDELRHPVFC DFGNSRWSIQ FAIHKEIRDR DKGAKQDTRQ LQNRHGLKMR LWNGRSMTDV |
| | 601 | NLHWSSKRLT ADLALDQNPN PNPTEVTRAD RLGRAASSAF DHVKIKNVFN EKEWNGRLQA |
| | 661 | PRAELDRIAK LEEQGKTEQA EKLRKRLRWY VSFSPCLSPS GPFIVYAGQH NIQPKRSGQY |
| | 721 | APHAQANKGR ARLAQLILSR LPDLRILSVD LGHRFAAACA VWETLSSDAF RREIQGLNVL |
| | 781 | AGGSGEGDLF LHVEMTGDDG KRRTVVYRRI GPDQLLDNTP HPAPWARLDR QFLIKLQGED |
| | 841 | EGVREASNEE LWTVHKLEVE VGRTVPLIDR MVRSGFGKTE KQKERLKKLR ELGWISAMPN |
| | 901 | EPSAETDEKE GEIRSISRSV DELMSSALGT LRLALKRHGN RARIAFAMTA DYKPMPGGQK |
| | 961 | YYFHEAKEAS KNDDETKRRD NQIEFLQDAL SLWHDLFSSP DWEDNEAKKL WQNHIATLPN |
| | 1021 | YQTPEEISAE LKRVERNKKR KENRDKLRTA AKALAENDQL RQHLHDTWKE RWESDDQQWK |
| | 1081 | ERLRSLKDWI FPRGKAEDNP SIRHVGGLSI TRINTISGLY QILKAFKMRP EPDDLRKNIP |
| | 1141 | QKGDDELENF NRRLLEARDR LREQRVKQLA SRIIEAALGV GRIKIPKNGK LPKRPRTTVD |
| | 1201 | TPCHAVVIES LKTYRPDDLR TRRENRQLMQ WSSAKVRKYL KEGCELYGLH FLEVPANYTS |
| | 1261 | RQCSRTGLPG IRCDDVPTGD FLKAPWWRRA INTAREKNGG DAKDRFLVDL YDHLNNLQSK |
| | 1321 | GEALPATVRV PRQGGNLFIA GAQLDDTNKE RRAIQADLNA AANIGLRALL DPDWRGRWWY |
| | 1381 | VPCKDGTSEP ALDRIEGSTA FNDVRSLPTG DNSSRRAPRE IENLWRDPSG DSLESGTWSP |
| | 1441 | TRAYWDTVQS RVIELLRRHA GLPTS (SEQ ID NO: 390) |
| LsCas12b | 1 | MSIRSFKLKL KTKSGVNAEQ LRRGLWRTHQ LINDGIAYYM NWLVLLRQED LFIRNKETNE |
| Laceyella | 61 | IEKRSKEEIQ AVLLERVHKQ QQRNQWSGEV DEQTLLQALR QLYEEIVPSV IGKSGNASLK |
| sacchari | 121 | ARFFLGPLVD PNNKTTKDVS KSGPTPKWKK MKDAGDPNWV QEYEKYMAER QTLVRLEEMG |
| WP_132221894. | 181 | LIPLFPMYTD EVGDIHWLPQ ASGYTRTWDR DMFQQAIERL LSWESWNRRV RERRAQFEKK |
| 1 | 241 | THDFASRFSE SDVQWMNKLR EYEAQQEKSL EENAFAPNEP YALTKKALRG WERVYHSWMR |
| | 301 | LDSAASEEAY WQEVATCQTA MRGEFGDPAI YQFLAQKENH DIWRGYPERV IDFAELNHLQ |
| | 361 | RELRRAKEDA TFTLPDSVDH PLWVRYEAPG GTNIHGYDLV QDTKRNLTLI LDKFILPDEN |
| | 421 | GSWHEVKKVP FSLAKSKQFH RQVWLQEEQK QKKREVVFYD YSTNLPHLGT LAGAKLQWDR |
| | 481 | NFLNKRTQQQ IEETGEIGKV FFNISVDVRP AVEVKNGRLQ NGLGKALTVL THPDGTKIVT |
| | 541 | GWKAEQLEKW VGESGRVSSL GLDSLSEGLR VMSIDLGQRT SATVSVFEIT KEAPDNPYKF |
| | 601 | FYQLEGTEMF AVHQRSFLLA LPGENPPQKI KQMREIRWKE RNRIKQQVDQ LSAILRLHKK |
| | 661 | VNEDERIQAI DKLLQKVASW QLNEEIATAW NQALSQLYSK AKENDLQWNQ AIKNAHHQLE |
| | 721 | PVVGKQISLW RKDLSTGRQG IAGLSLWSIE ELEATKKLLT RWSKRSREPG VVKRIERFET |
| | 781 | FAKQIQHHIN QVKENRLKQL ANLIVMTALG YKYDQEQKKW IEVYPACQVV LFENLRSYRF |
| | 841 | SFERSRRENK KLMEWSHRSI PKLVQMQGEL FGLQVADVYA AYSSRYHGRT GAPGIRCHAL |
| | 901 | TEADLRNETN IIHELIEAGF IKEEHRPYLQ QGDLVPWSGG ELFATLQKPY DNPRILTLHA |
| | 961 | DINAAQNIQK RFWHPSMWFR VNCESVMEGE IVTYVPKNKT VHKKQGKTFR FVKVEGSDVY |
| | 1021 | EWAKWSKNRN KNTFSSITER KPPSSMILFR DPSGTFFKEQ EWVEQKTFWG KVQSMIQAYM |
| | 1081 | KKTIVQRMEE (SEQ ID NO: 391) |
| DtCas12b | 1 | MVLGRKDDTA ELRRALWTTH EHVNLAVAEV ERVLLRCRGR SYWTLDRRGD PVHVPESQVA |
| Dsulfonatronum | 61 | EDALAMAREA QRRNGWPVVG EDEEILLALR YLYEQIVPSC LLDDLGKPLK GDAQKIGTNY |
| thiodismutans | 121 | AGPLFDSDTC RRDEGKDVAC CGPFHEVAGK YLGALPEWAT PISKQEFDGK DASHLRFKAT |
| WP_031386437 | 181 | GGDDAFFRVS IEKANAWYED PANQDALKNK AYNKDDWKKE KDKGISSWAV KYIQKQLQLG |
| | 241 | QDPRTEVRRK LWLELGLLPL FIPVFDKTMV GNLWNRLAVR LALAHLLSWE SWNHRAVQDQ |
| | 301 | ALARAKRDEL AALFLGMEDG FAGLREYELR RNESIKQHAF EPVDRPYVVS GRALRSWTRV |
| | 361 | REEWLRHGDT QESRKNICNR LQDRLRGKFG DPDVFHWLAE DGQEALWKER DCVTSFSLLN |
| | 421 | DADGLLEKRK GYALMTFADA RLHPRWAMYE APGGSNLRTY QIRKTENGLW ADVVLLSPRN |
| | 481 | ESAAVEEKTE NVRLAPSGQL SNVSFDQIQK GSKMVGRCRY QSANQQFEGL LGGAEILFDR |
| | 541 | KRIANEQHGA TDLASKPGHV WFKLTLDVRP QAPQGWLDGK GRPALPPEAK HFKTALSNKS |
| | 601 | KFADQVRPGL RVLSVDLGVR SFAACSVFEL VRGGPDQGTY FPAADGRTVD DPEKLWAKHE |
| | 661 | RSFKITLPGE NPSRKEEIAR RAAMEELRSL NGDIRRLKAI LRLSVLQEDD PRTEHLRLFM |
| | 721 | EAIVDDPAKS ALNAELFKGF GDDRFRSTPD LWKQHCHFFH DKAEKVVAER FSRWRTETRP |
| | 781 | KSSSWQDWRE RRGYAGGKSY WAVTYLEAVR GLILRWNMRG RTYGEVNRQD KKQFGTVASA |
| | 841 | LLHHINQLKE DRIKTGADMI IQAARGFVPR KNGAGWVQVH EPCRLILFED LARYRFRTDR |
| | 901 | SRRENSRLMR WSHREIVNEV GMQGELYGLH VDTTEAGFSS RYLASSGAPG VRCRHLVEED |
| | 961 | FHDGLPGMHL VGELDWLLPK DKDRTANEAR RLLGGMVRPG MLVPWDGGEL FATLNAASQL |
| | 1021 | HVIHADINAA QNLQRRFWGR CGEAIRIVCN QLSVDGSTRY EMAKAPKARL LGALQQLKNG |
| | 1081 | DAPFHLTSIP NSQKPENSYV MTPTNAGKKY RAGPGEKSSG EEDELALDIV EQAEELAQGR |
| | 1141 | KTFFRDPSGV FFAPDRWLPS EIYWSRIRRR IWQVTLERNS SGRQERAEMD EMPY |
| | | (SEQ ID NO: 392) |

The adenine base editors described herein may also comprise Cas12a/Cpf1 (dCpf1) variants that may be used as a guide nucleotide sequence-programmable DNA-binding protein domain. The Cas12a/Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity.

G. napDNAbps that Recognize Non-Canonical PAM Sequences

In some embodiments, the napDNAbp is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 July; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference.

In some embodiments, the disclosure provides napDNAbp domains that comprise SpCas9 variants that recognize and work best with NRRH, NRCH, and NRTH PAMs. See International Application No. PCT/US2019/47996, which published as International Publication No. WO 2020/041751 on Feb. 27, 2020, incorporated by reference herein. In some embodiments, the disclosed base editors comprise a napDNAbp domain selected from SpCas9-NRRH, SpCas9-NRTH, and SpCas9-NRCH.

In some embodiments, the disclosed base editors comprise a napDNAbp domain that has a sequence that is at least 90%, at least 95%, at least 98%, or at least 99% identical to SpCas9-NRRH. In some embodiments, the disclosed base editors comprise a napDNAbp domain that comprises SpCas9-NRRH. The SpCas9-NRRH has an amino acid sequence as presented in SEQ ID NO: 435 (underlined residues are mutated relative to SpCas9, as set forth in SEQ ID NO: 326)

(SEQ ID NO: 435)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMVKRYDEHHQDLTLLKALVRQQLPEKYKEI
FFDQSKNGYAGYIDGGASQEEFYKFIKPILEKMDGTEELLVKLNREDLLR
KQRTFDNGIIPHQIHLGELHAILRRQGDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRLRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGGHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKGNSDKLIARKKDWDPKKYGGFNSPTAAYSVLVVAKVE
KGKSKKLKSVKELLGITIMERSSFEKNPIGFLEAKGYKEVKKDLIIKLPK
YSLFELENGRKRMLASAGVLHKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGVPAAFKYFDTTIDKKRYTSTKEVLDATLIHQ
SITGLYETRIDLSQLGGD.

In some embodiments, the disclosed base editors comprise a napDNAbp domain that has a sequence that is at least 90%, at least 95%, at least 98%, or at least 99% identical to SpCas9-NRCH. In some embodiments, the disclosed base editors comprise a napDNAbp domain that comprises SpCas9-NRCH. An example of an NRCH PAM is CACC (5′-CACC-3′). The SpCas9-NRCH has an amino acid sequence as presented in SEQ ID NO: 436 (underlined residues are mutated relative to SpCas9)

(SEQ ID NO: 436)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMVKRYDEHHQDLTLLKALVRQQLPEKYKEI
FFDQSKNGYAGYIDGGASQEEFYKFIKPILEKMDGTEELLVKLNREDLLR
KQRTFDNGIIPHQIHLGELHAILRRQGDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRLRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGGHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKGNSDKLIARKKDWDPKKYGGFNSPTVAYSVLVVAKVE
KGKSKKLKSVKELLGITIMERSSFEKNPIDFLEAKGYKEVKKDLIIKLPK
YSLFELENGRKRMLASAGVLQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTINRKQYNTTKEVLDATLIRQ
SITGLYETRIDLSQLGGD

In some embodiments, the disclosed base editors comprise a napDNAbp domain that has a sequence that is at least 90%, at least 95%, at least 98%, or at least 99% identical to SpCas9-NRTH. In some embodiments, the disclosed base editors comprise a napDNAbp domain that comprises SpCas9-NRTH. The SpCas9-NRTH has an amino acid sequence as presented in SEQ ID NO: 437 (underlined residues are mutated relative to SpCas9)

(SEQ ID NO: 437)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMVKRYDEHHQDLTLLKALVRQQLPEKYKEI
FFDQSKNGYAGYIDGGASQEEFYKFIKPILEKMDGTEELLVKLNREDLLR
KQRTFDNGIIPHQIHLGELHAILRRQGDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRLRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGGHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKGNSDKLIARKKDWDPKKYGGFNSPTVAYSVLVVAKVE
KGKSKKLKSVKELLGITIMERSSFEKNPIGFLEAKGYKEVKKDLIIKLPK
YSLFELENGRKRMLASASVLHKGNELALPSKYVNFLYLASHYEKLKGSSE
DNKQKQLFVEQHKHYLDEIIEQISEFSKRVILADANLDKVLSAYNKHRDK
PIREQAENIIHLFTLTNLGASAAFKYFDTTIGRKLYTSTKEVLDATLIHQ
SITGLYETRIDLSQLGGD

In other embodiments, the napDNAbp of any of the disclosed base editors comprises a Cas9 derived from a Streptococcus macacae, e.g. Streptococcus macacae NCTC 11558, or SmacCas9, or a variant thereof. In some embodiments, the napDNAbp comprises a hybrid variant of SmacCas9 that incorporates an SpCas9 domain with the SmacCas9 domain and is known as Spy-macCas9, or a variant thereof. In some embodiments, the napDNAbp comprises a hybrid variant of SmacCas9 that incorporates an increased nucleolytic variant of an SpCas9 (iSpy Cas9) domain and is known as iSpy-macCas9. Relative to Spymac-Cas9, iSpyMac-Cas9 contains two mutations, R221K and N394K, that were identified by deep mutational scans of Spy Cas9 that raise modification rates of the protein on most targets. See Jakimo et al., bioRxiv, A Cas9 with Complete PAM Recognition for Adenine Dinucleotides (September 2018), herein incorporated by reference. Jakimo et al. showed that the hybrids Spy-macCas9 and iSpy-macCas9 recognize a short 5′-NAA-3′ PAM and recognized all evaluated adenine dinucleotide PAM sequences and possesses robust editing efficiency in human cells. Liu et al. engineered base editors containing Spy-mac Cas9, and demonstrated that cytidine and adenine base editors containing Spymac domains can induce efficient C-to-T and A-to-G conversions in vivo. In addition, Liu et al. suggested that the PAM scope of Spy-mac Cas9 may be 5′-TAAA-3′, rather than 5′-NAA-3′ as reported by Jakimo et al (see Liu et al. Cell Discovery (2019) 5:58, herein incorporated by reference).

In some embodiments, the disclosed base editors comprise a napDNAbp domain that has a sequence that is at least 90%, at least 95%, at least 98%, or at least 99% identical to iSpyMac-Cas9. In some embodiments, the disclosed base editors comprise a napDNAbp domain that comprises iSpyMac-Cas9 (or SpyMac-Cas9). The iSpyMac-Cas9 has an amino acid sequence as presented in SEQ ID NO: 439 (R221K and N394K mutations are underlined):

(SEQ ID NO: 439)
DKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFE
ENPINASGVDAKAILSARLSKSRKLENLIAQLPGEKKNGLFGNLIALSL
GLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLP
EKYKEIFFDQSKNGYAGYIDGGASQEEFYKFIKPILEKMDGTEELLVKL
KREDLLRKQRTFDNGSIPHQIHLGELHAILRRQEDFYPFLKDNREKIEK
ILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIE
EGIKELGSQILKEHPVENTQLQNEKLYLYYLQNGRDMYVDQELDINRLS
DYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVA
QILDSRMNTKYDENDKLIREVKVITLKSKLVSDFRKDFQFYKVREINNY
HHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRD
FATVRKVLSMPQVNIVKKTEIQTVGQNGGLFDDNPKSPLEVTPSKLVPL
KKELNPKKYGGYQKPTTAYPVLLITDTKQLIPISVMNKKQFEQNPVKFL
RDRGYQQVGKNDFIKLPKYTLVDIGDGIKRLWASSKEIHKGNQLVVSKK
SQILLYHAHHLDSDLSNDYLQNHNQQFDVLFNEIISFSKKCKLGKEHIQ
KIENVYSNKKNSASIEELAESFIKLLGFTQLGATSPFNFLGVKLNQKQY
KGKKDYILPCTEGTLIRQSITGLYETRVDLSKIGED

In other embodiments, the napDNAbp of any of the disclosed base editors is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. 2009 Aug. 25; 4:29. doi: 10.1186/1745-6150-4-29, the entire contents of which is hereby incorporated by reference. In some embodiments, the napDNAbp is a Marinitoga piezophila Argunaute (MpAgo) protein. The CRISPR-associated Marinitoga piezophila Argunaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, the entire contents of which are hereby incorporated by reference). It should be appreciated that other argonaute proteins may be used, and are within the scope of this disclosure.

In some embodiments, the napDNAbp is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, C2c1 and C2c3, contain RuvC-like endonuclease domains related to Cpf1. A third system, C2c2 contains an effector with two predicated HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13; 538(7624):270-273, the entire contents of which are hereby incorporated by reference. In vitro biochemical analysis of C2c2 in Leptotrichia shahii has shown that C2c2 is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector”, Science, 2016 Aug. 5; 353(6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the napDNAbp may be a C2c1, a C2c2, or a C2c3 protein. In some embodiments, the napDNAbp is a C2c1 protein. In some embodiments, the napDNAbp is a C2c2 protein. In some embodiments, the napDNAbp is a C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the napDNAbp is a naturally-occurring C2c1, C2c2, or C2c3 protein.

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing base editors provided herein may need to be placed at a precise location, for example where a target base is placed within a 4 base region (e.g., a “editing window” or a “target window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the base editors provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

For example, a napDNAbp domain with altered PAM specificity, such as a domain with at least 80%, at least 85%, at least 90%, at least 95%, or at least 99% sequence identity with wild type Francisella novicida Cpf1 (SEQ ID NO: 393) (D917, E1006, and D1255), which has the following amino acid sequence:

(SEQ ID NO: 393)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN

An additional napDNAbp domain with altered PAM specificity, such as a domain having at least 80%, at least 85%, at least 90%, at least 95%, or at least 99% sequence identity with wild type Geobacillus thermodenitrificans Cas9 (SEQ ID NO: 394), which has the following amino acid sequence:

(SEQ ID NO: 394)
MKYKIGLDIGITSIGWAVINLDIPRIEDLGVRIFDRAENPKTGESLALPR
RLARSARRRLRRRKHRLERIRRLFVREGILTKEELNKLFEKKHEIDVWQL
RVEALDRKLNNDELARILLHLAKRRGFRSNRKSERTNKENSTMLKHIEEN
QSILSSYRTVAEMVVKDPKFSLHKRNKEDNYTNTVARDDLEREIKLIFAK
QREYGNIVCTEAFEHEYISIWASQRPFASKDDIEKKVGFCTFEPKEKRAP
KATYTFQSFTVWEHINKLRLVSPGGIRALTDDERRLIYKQAFHKNKITFH
DVRTLLNLPDDTRFKGLLYDRNTTLKENEKVRFLELGAYHKIRKAIDSVY
GKGAAKSFRPIDFDTFGYALTMFKDDTDIRSYLRNEYEQNGKRMENLADK
VYDEELIEELLNLSFSKFGHLSLKALRNILPYMEQGEVYSTACERAGYTF
TGPKKKQKTVLLPNIPPIANPVVMRALTQARKVVNAIIKKYGSPVSIHIE
LARELSQSFDERRKMQKEQEGNRKKNETAIRQLVEYGLTLNPTGLDIVKF
KLWSEQNGKCAYSLQPIEIERLLEPGYTEVDHVIPYSRSLDDSYTNKVLV
LTKENREKGNRTPAEYLGLGSERWQQFETFVLTNKQFSKKKRDRLLRLHY
DENEENEFKNRNLNDTRYISRFLANFIREHLKFADSDDKQKVYTVNGRIT
AHLRSRWNFNKNREESNLHHAVDAAIVACTTPSDIARVTAFYQRREQNKE
LSKKTDPQFPQPWPHFADELQARLSKNPKESIKALNLGNYDNEKLESLQP
VFVSRMPKRSITGAAHQETLRRYIGIDERSGKIQTVVKKKLSEIQLDKTG
HFPMYGKESDPRTYEAIRQRLLEHNNDPKKAFQEPLYKPKKNGELGPIIR
TIKIIDTTNQVIPLNDGKTVAYNSNIVRVDVFEKDGKYYCVPIYTIDMMK
GILPNKAIEPNKPYSEWKEMTEDYTFRFSLYPNDLIRIEFPREKTIKTAV
GEEIKIKDLFAYYQTIDSSNGGLSLVSHDNNFSLRSIGSRTLKRFEKYQV
DVLGNIYKVRGEKRVGVASSSHSKAGETIRPL

In some embodiments, the nucleic acid programmable DNA binding protein (napDNAbp) is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 34(7): 768-73 (2016), PubMed PMID: 27136078; Swarts et al., Nature, 507(7491): 258-61 (2014); and Swarts et al., Nucleic Acids Res. 43(10) (2015): 5120-9, each of which is incorporated herein by reference. The sequence of Natronobacterium gregoryi Argonaute is provided in SEQ ID NO: 813095.

The disclosed base editors may comprise a napDNAbp domain having at least 80%, at least 85%, at least 90%, at least 95%, or at least 99% sequence identity with wild type Natronobacterium gregoryi Argonaute (SEQ ID NO: 395), which has the following amino acid sequence:

(SEQ ID NO: 395)
MTVIDLDSTTTADELTSGHTYDISVTLTGVYDNTDEQHPRMSLAFEQDNG
ERRYITLWKNTTPKDVFTYDYATGSTYIFTNIDYEVKDGYENLTATYQTT
VENATAQEVGTTDEDETFAGGEPLDHHLDDALNETPDDAETESDSGHVMT
SFASRDQLPEWTLHTYTLTATDGAKTDTEYARRTLAYTVRQELYTDHDAA
PVATDGLMLLTPEPLGETPLDLDCGVRVEADETRTLDYTTAKDRLLAREL
VEEGLKRSLWDDYLVRGIDEVLSKEPVLTCDEFDLHERYDLSVEVGHSGR
AYLHINFRHRFVPKLTLADIDDDNIYPGLRVKTTYRPRRGHIVWGLRDEC
ATDSLNTLGNQSVVAYHRNNQTPINTDLLDAIEAADRRVVETRRQGHGDD
AVSFPQELLAVEPNTHQIKQFASDGFHQQARSKTRLSASRCSEKAQAFAE
RLDPVRLNGSTVEFSSEFFTGNNEQQLRLLYENGESVLTFRDGARGAHPD
ETFSKGIVNPPESFEVAVVLPEQQADTCKAQWDTMADLLNQAGAPPTRSE
TVQYDAFSSPESISLNVAGAIDPSEVDAAFVVLPPDQEGFADLASPTETY
DELKKALANMGIYSQMAYFDRFRDAKIFYTRNVALGLLAAAGGVAFTTEH
AMPGDADMFIGIDVSRSYPEDGASGQINIAATATAVYKDGTILGHSSTRP
QLGEKLQSTDVRDIMKNAILGYQQVTGESPTHIVIHRDGFMNEDLDPATE
FLNEQGVEYDIVEIRKQPQTRLLAVSDVQYDTPVKSIAAINQNEPRATVA
TFGAPEYLATRDGGGLPRPIQIERVAGETDIETLTRQVYLLSQSHIQVHN
STARLPITTAYADQASTHATKGYLVQTGAFESNVGFL

H. Cas9 Circular Permutants

In various embodiments, the adenine base editors disclosed herein may comprise a circular permutant of Cas9.

The term “circularly permuted Cas9” or “circular permutant” of Cas9 or “CP-Cas9”) refers to any Cas9 protein, or variant thereof, that occurs or has been modify to engineered as a circular permutant variant, which means the N-terminus and the C-terminus of a Cas9 protein (e.g., a wild type Cas9 protein) have been topically rearranged. Such circularly permuted Cas9 proteins, or variants thereof, retain the ability to bind DNA when complexed with a guide RNA (gRNA). See, Oakes et al., “Protein Engineering of Cas9 for enhanced function,” Methods Enzymol, 2014, 546: 491-511 and Oakes et al., “CRISPR-Cas9 Circular Permutants as Programmable Scaffolds for Genome Modification,” Cell, Jan. 10, 2019, 176: 254-267, and Huang, T. P. et al. Circularly permuted and PAM-modified Cas9 variants broaden the targeting scope of base editors. Nat. Biotechnol. 37, 626-631 (2019). each of are incorporated herein by reference. Reference is also made to International Publication No. WO 2020/041751, published Feb. 27, 2020, herein incorporated by reference. The present disclosure contemplates any previously known CP-Cas9 or use a new CP-Cas9 so long as the resulting circularly permuted protein retains the ability to bind DNA when complexed with a guide RNA (gRNA).

Any of the Cas9 proteins described herein, including any variant, ortholog, or naturally occurring Cas9 or equivalent thereof, may be reconfigured as a circular permutant variant.

In various embodiments, the circular permutants of Cas9 may have the following structure: N-terminus-[original C-terminus]-[optional linker]-[original N-terminus]-C-terminus.

As an example, the present disclosure contemplates the following circular permutants of canonical S. pyogenes Cas9 (1368 amino acids of UniProtKB—Q99ZW2 (CAS9_STRP1) (numbering is based on the amino acid position in SEQ ID NO: 326)): N-terminus-[1268-1368]-[optional linker]-[1-1267]-C-terminus; N-terminus-[1168-1368]-[optional linker]-[1-1167]-C-terminus; N-terminus-[1068-1368]-[optional linker]-[1-1067]-C-terminus; N-terminus-[968-1368]-[optional linker]-[1-967]-C-terminus; N-terminus-[868-1368]-[optional linker]-[1-867]-C-terminus; N-terminus-[768-1368]-[optional linker]-[1-767]-C-terminus; N-terminus-[668-1368]-[optional linker]-[1-667]-C-terminus; N-terminus-[568-1368]-[optional linker]-[1-567]-C-terminus; N-terminus-[468-1368]-[optional linker]-[1-467]-C-terminus; N-terminus-[368-1368]-[optional linker]-[1-367]-C-terminus; N-terminus-[268-1368]-[optional linker]-[1-267]-C-terminus; N-terminus-[168-1368]-[optional linker]-[1-167]-C-terminus; N-terminus-[68-1368]-[optional linker]-[1-67]-C-terminus; or N-terminus-[10-1368]-[optional linker]-[1-9]-C-terminus, or the corresponding circular permutants of other Cas9 proteins (including other Cas9 orthologs, variants, etc).

In particular embodiments, the circular permuant Cas9 has the following structure (based on S. pyogenes Cas9 (1368 amino acids of UniProtKB—Q99ZW2 (CAS9_STRP1) (numbering is based on the amino acid position in SEQ ID NO: 326): N-terminus-[102-1368]-[optional linker]-[1-101]-C-terminus; N-terminus-[1028-1368]-[optional linker]-[1-1027]-C-terminus; N-terminus-[1041-1368]-[optional linker]-[1-1043]-C-terminus; N-terminus-[1249-1368]-[optional linker]-[1-1248]-C-terminus; or N-terminus-[1300-1368]-[optional linker]-[1-1299]-C-terminus, or the corresponding circular permutants of other Cas9 proteins (including other Cas9 orthologs, variants, etc).

In still other embodiments, the circular permuant Cas9 has the following structure (based on S. pyogenes Cas9 (1368 amino acids of UniProtKB—Q99ZW2 (CAS9_STRP1) (numbering is based on the amino acid position in SEQ ID NO: 326): N-terminus-[103-1368]-[optional linker]-[1-102]-C-terminus; N-terminus-[1029-1368]-[optional linker]-[1-1028]-C-terminus; N-terminus-[1042-1368]-[optional linker]-[1-1041]-C-terminus; N-terminus-[1250-1368]-[optional linker]-[1-1249]-C-terminus; or N-terminus-[1301-1368]-[optional linker]-[1-1300]-C-terminus, or the corresponding circular permutants of other Cas9 proteins (including other Cas9 orthologs, variants, etc.).

In some embodiments, the circular permutant can be formed by linking a C-terminal fragment of a Cas9 to an N-terminal fragment of a Cas9, either directly or by using a linker, such as an amino acid linker. In some embodiments, The C-terminal fragment may correspond to the C-terminal 95% or more of the amino acids of a Cas9 (e.g., amino acids about 1300-1368), or the C-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., any one of SEQ ID NOs: 18-25). The N-terminal portion may correspond to the N-terminal 95% or more of the amino acids of a Cas9 (e.g., amino acids about 1-1300), or the N-terminal 90%, 85%, 80%, 75%, 70%, 65%, 60%, 55%, 50%, 45%, 40%, 35%, 30%, 25%, 20%, 15%, 10%, or 5% or more of a Cas9 (e.g., of SEQ ID NO: 326).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus, includes or corresponds to the C-terminal 30% or less of the amino acids of a Cas9 (e.g., amino acids 1012-1368 of SEQ ID NO: 326). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., the Cas9 of SEQ ID NO: 326). In some embodiments, the C-terminal fragment that is rearranged to the N-terminus, includes or corresponds to the C-terminal 410 residues or less of a Cas9 (e.g., the Cas9 of SEQ ID NO: 326). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., the Cas9 of SEQ ID NO: 326). In some embodiments, the C-terminal portion that is rearranged to the N-terminus, includes or corresponds to the C-terminal 357, 341, 328, 120, or 69 residues of a Cas9 (e.g., the Cas9 of SEQ ID NO: 326).

In other embodiments, circular permutant Cas9 variants may be defined as a topological rearrangement of a Cas9 primary structure based on the following method, which is based on S. pyogenes Cas9 of SEQ ID NO: 326: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to preceed the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (relative the S. pyogenes Cas9 of SEQ ID NO: 326) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9_CP³¹⁰, Cas9-CP¹⁰¹⁰, Cas9-CP¹⁰¹⁶, Cas9-CP¹⁰²³, Cas9-CP¹⁰²⁹, Cas9_CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 326, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

Exemplary CP-Cas9 amino acid sequences, based on the Cas9 of SEQ ID NO: 326, are provided below in which linker sequences are indicated by underlining and optional methionine (M) residues are indicated in bold. It should be appreciated that the disclosure provides CP-Cas9 sequences that do not include a linker sequence or that include different linker sequences. It should be appreciated that CP-Cas9 sequences may be based on Cas9 sequences other than that of SEQ ID NO: 326 and any examples provided herein are not meant to be limiting. Exemplary CP-Cas9 sequences are as follows:

| CP name | Sequence | SEQ ID NO: |
|---|---|---|
| CP1012 | DYKVYDVRKMIAKSEQEIGKATAKYFFYSNIMNFFKTEITLANGEIRKRPLIETN | SEQ ID NO: |
| | GETGEIVWDKGRDFATVRKVLSMPQVNIVKKTEVQTGGFSKESILPKRNSDKLIA | 396 |
| | RKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKE | |
| | VLDATLIHQSITGLYETRIDLSQLGGDGGSGGSGGSGGSGGSGGSGGDKKYSIGL | |
| | AIGTNSVGWAVITDEYKVPSKKFKVLGNTDRHSIKKNLIGALLFDSGETAEATRL | |
| | KRTARRRYTRRKNRICYLQEIFSNEMAKVDDSFFHRLEESFLVEEDKKHERHPIF | |
| | GNIVDEVAYHEKYPTIYHLRKKLVDSTDKADLRLIYLALAHMIKFRGHFLIEGDL | |
| | NPDNSDVDKLFIQLVQTYNQLFEENPINASGVDAKAILSARLSKSRRLENLIAQL | |
| | PGEKKNGLFGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDLTLLKALV | |
| | RQQLPEKYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLPN | |
| | EKVLPKHSLLYEYFTVYNELTKVKYVTEGMRKPAFLSGEQKKAIVDLLFKTNRKV | |
| | TVKQLKEDYFKKIECFDSVEISGVEDRFNASLGTYHDLLKIIKDKDFLDNEENED | |
| | ILEDIVLTLTLFEDREMIEERLKTYAHLFDDKVMKQLKRRRYTGWGRLSRKLING | |
| | IRDKQSGKTILDFLKSDGFANRNFMQLIHDDSLTFKEDIQKAQVSGQGDSLHEHI | |
| | ANLAGSPAIKKGILQTVKVVDELVKVMGRHKPENIVIEMARENQTTQKGQKNSRE | |
| | RMKRIEEGIKELGSQILKEHPVENTQLQNEKLYLYYLQNGRDMYVDQELDINRLS | |
| | DYDVDHIVPQSFLKDDSIDNKVLTRSDKNRGKSDNVPSEEVVKKMKNYWRQLLNA | |
| | KLITQRKFDNLTKAERGGLSELDKAGFIKRQLVETRQITKHVAQILDSRMNTKYD | |
| | ENDKLIREVKVITLKSKLVSDFRKDFQFYKVREINNYHHAHDAYLNAVVGTALIK | |
| | KYPKLESEFVYG | |
| CP1028 | EIGKATAKYFFYSNIMNFFKTEITLANGEIRKRPLIETNGETGEIVWDKGRDFAT | SEQ ID NO: |
| | VRKVLSMPQVNIVKKTEVQTGGFSKESILPKRNSDKLIARKKDWDPKKYGGFDSP | 397 |
| | TVAYSVLVVAKVEKGKSKKLKSVKELLGITIMERSSFEKNPIDFLEAKGYKEVKK | |
| | DLIIKLPKYSLFELENGRKRMLASAGELQKGNELALPSKYVNFLYLASHYEKLKG | |
| | SPEDNEQKQLFVEQHKHYLDEIIEQISEFSKRVILADANLDKVLSAYNKHRDKPI | |
| | REQAENIIHLFTLTNLGAPAAFKYFDTTIDRKRYTSTKEVLDATLIHQSITGLYE | |
| | TRIDLSQLGGDGGSGGSGGSGGSGGSGGSGGMDKKYSIGLAIGTNSVGWAVITDE | |
| | YKVPSKKFKVLGNTDRHSIKKNLIGALLFDSGETAEATRLKRTARRRYTRRKNRI | |
| | CYLQEIFSNEMAKVDDSFFHRLEESFLVEEDKKHERHPIFGNIVDEVAYHEKYPT | |
| | IYHLRKKLVDSTDKADLRLIYLALAHMIKFRGHFLIEGDLNPDNSDVDKLFIQLV | |
| | QTYNQLFEENPINASGVDAKAILSARLSKSRRLENLIAQLPGEKKNGLFGNLIAL | |
| | SLGLTPNFKSNFDLAEDAKLQLSKDTYDDDLDNLLAQIGDQYADLFLAAKNLSDA | |
| | ILLSDILRVNTEITKAPLSASMIKRYDEHHQDLTLLKALVRQQLPEKYKEIFFDQ | |
| | SKNGYAGYIDGGASQEEFYKFIKPILEKMDGTEELLVKLNREDLLRKQRTFDNGS | |
| | IPHQIHLGELHAILRRQEDFYPFLKDNREKIEKILTFRIPYYVGPLARGNSRFAW | |
| | MTRKSEETITPWNFEEVVDKGASAQSFIERMTNFDKNLPNEKVLPKHSLLYEYFT | |
| | VYNELTKVKYVTEGMRKPAFLSGEQKKAIVDLLFKTNRKVTVKQLKEDYFKKIEC | |
| | FDSVEISGVEDRFNASLGTYHDLLKIIKDKDFLDNEENEDILEDIVLTLTLFEDR | |
| | EMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIKELGSQ | |
| | ILKEHPVENTQLQNEKLYLYYLQNGRDMYVDQELDINRLSDYDVDHIVPQSFLKD | |
| | DSIDNKVLTRSDKNRGKSDNVPSEEVVKKMKNYWRQLLNAKLITQRKFDNLTKAE | |
| | RGGLSELDKAGFIKRQLVETRQITKHVAQILDSRMNTKYDENDKLIREVKVITLK | |
| | SKLVSDFRKDFQFYKVREINNYHHAHDAYLNAVVGTALIKKYPKLESEFVYGDYK | |
| | VYDVRKMIAKSEQ | |
| CP1041 | NIMNFFKTEITLANGEIRKRPLIETNGETGEIVWDKGRDFATVRKVLSMPQVNIV | SEQ ID NO: |
| | KKTEVQTGGFSKESILPKRNSDKLIARKKDWDPKKYGGFDSPTVAYSVLVVAKVE | 398 |
| | KGKSKKLKSVKELLGITIMERSSFEKNPIDFLEAKGYKEVKKDLIIKLPKYSLFE | |
| | LENGRKRMLASAGELQKGNELALPSKYVNFLYLASHYEKLKGSPEDNEQKQLFVE | |
| | QHKHYLDEIIEQISEFSKRVILADANLDKVLSAYNKHRDKPIREQAENIIHLFTL | |
| | TNLGAPAAFKYFDTTIDRKRYTSTKEVLDATLIHQSITGLYETRIDLSQLGGDGG | |
| | SGGSGGSGGSGGSGGSGGDKKYSIGLAIGTNSVGWAVITDEYKVPSKKFKVLGNT | |
| | DRHSIKKNLIGALLFDSGETAEATRLKRTARRRYTRRKNRICYLQEIFSNEMAKV | |
| | DDSFFHRLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFEENPINA | |
| | SGVDAKAILSARLSKSRRLENLIAQLPGEKKNGLFGNLIALSLGLTPNFKSNFDL | |
| | AEDAKLQLSKDTYDDDLDNLLAQIGDQYADLFLAAKNLSDAILLSDILRVNTEIT | |
| | KAPLSASMIKRYDEHHQDLTLLKALVRQQLPEKYKEIFFDQSKNGYAGYIDGGAS | |
| | QEEFYKFIKPILEKMDGTEELLVKLNREDLLRKQRTFDNGSIPHQIHLGELHAIL | |
| | RRQEDFYPFLKDNREKIEKILTFRIPYYVGPLARGNSRFAWMTRKSEETITPWNF | |
| | EEVVDKGASAQSFIERMTNFDKNLPNEKVLPKHSLLYEYFTVYNELTKVKYVTEG | |
| | MRKPAFLSGEQKKAIVDLLFKTNRKVTVKQLKEDYFKKIECFDSVEISGVEDRFN | |
| | ASLGTYHDLLKIIKDKDFLDNEENEDILEDIVLTLTLFEDREMIEERLKTYAHLF | |
| | DDKVMKQLKRRRYTGWGRLSRKLINGIRDKQSGKTILDFLKSDGFANRNFMQLIH | |
| | DDSLTFKEDIQKAQVSGQGDSLHEHIANLAGSPAIKKGILQTVKVVDELVKVMGR | |
| | HKPENIVIEMARENQTTQKGQKNSRERMKRIEEGIKELGSQILKEHPVENTQLQN | |
| | EKLYLYYLQNGRDMYVDQELDINRLSDYDVDHIVPQSFLKDDSIDNKVLTRSDKN | |
| | RGKSDNVPSEEVVKKMKNYWRQLLNAKLITQRKFDNLTKAERGGLSELDKAGFIK | |
| | RQLVETRQITKHVAQILDSRMNTKYDENDKLIREVKVITLKSKLVSDFRKDFQFY | |
| | KVREINNYHHAHDAYLNAVVGTALIKKYPKLESEFVYGDYKVYDVRKMIAKSEQE | |
| | IGKATAKYFFYS | |
| CP1249 | PEDNEQKQLFVEQHKHYLDEIIEQISEFSKRVILADANLDKVLSAYNKHRDKPIR | SEQ ID NO: |
| | EQAENIIHLFTLTNLGAPAAFKYFDTTIDRKRYTSTKEVLDATLIHQSITGLYET | 399 |
| | RIDLSQLGGDGGSGGSGGSGGSGGSGGSGGMDKKYSIGLAIGTNSVGWAVITDEY | |
| | KVPSKKFKVLGNTDRHSIKKNLIGALLFDSGETAEATRLKRTARRRYTRRKNRIC | |
| | YLQEIFSNEMAKVDDSFFHRLEESFLVEEDKKHERHPIFGNIVDEVAYHEKYPTI | |
| | YHLRKKLVDSTDKADLRLIYLALAHMIKFRGHFLIEGDLNPDNSDVDKLFIQLVQ | |
| | TYNQLFEENPINASGVDAKAILSARLSKSRRLENLIAQLPGEKKNGLFGNLIALS | |
| | LGLTPNFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEIFFDQS | |
| | KNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWM | |
| | TRKSEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKIECF | |
| | DSVEISGVEDRFNASLGTYHDLLKIIKDKDFLDNEENEDILEDIVLTLTLFEDRE | |
| | MIEERLKTYAHLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSPAIKKGILQT | |
| | VKVVDELVKVMGRHKPENIVIEMARENQTTQKGQKNSRERMKRIEEGIKELGSQI | |
| | LKEHPVENTQLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRKFDNLTKAER | |
| | GGLSELDKAGFIKRQLVETRQITKHVAQILDSRMNTKYDENDKLIREVKVITLKS | |
| | KLVSDFRKDFQFYKVREINNYHHAHDAYLNAVVGTALIKKYPKLESEFVYGDYKV | |
| | YDVRKMIAKSEQEIGKATAKYFFYSNIMNFFKTEITLANGEIRKRPLIETNGETG | |
| | EIVWDKGRDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSSFEKNPID | |
| | FLEAKGYKEVKKDLIIKLPKYSLFELENGRKRMLASAGELQKGNELALPSKYVNE | |
| | LYLASHYEKLKGS | |
| CP1300 | KPIREQAENIIHLFTLTNLGAPAAFKYFDTTIDRKRYTSTKEVLDATLIHQSITG | SEQ ID NO: |
| | LYETRIDLSQLGGDGGSGGSGGSGGSGGSGGSGGDKKYSIGLAIGTNSVGWAVIT | 400 |
| | DEYKVPSKKFKVLGNTDRHSIKKNLIGALLFDSGETAEATRLKRTARRRYTRRKN | |
| | RICYLQEIFSNEMAKVDDSFFHRLEESFLVEEDKKHERHPIFGNIVDEVAYHEKY | |
| | PTIYHLRKKLVDSTDKADLRLIYLALAHMIKFRGHFLIEGDLNPDNSDVDKLFIQ | |
| | LVQTYNQLFEENPINASGVDAKAILSARLSKSRRLENLIAQLPGEKKNGLFGNLI | |
| | ALSLGLTPNFKSNFDLAEDAKLQLSKDTYDDDLDNLLAQIGDQYADLFLAAKNLS | |
| | DAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDN | |
| | GSIPHQIHLGELHAILRRQEDFYPFLKDNREKIEKILTFRIPYYVGPLARGNSRF | |
| | AWMTRKSEETITPWNFEEVVDKGASAQSFIERMTNFDKNLPNEKVLPKHSLLYEY | |
| | FTVYNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDF | |
| | LKSDGFANRNFMQLIHDDSLTFKEDIQKAQVSGQGDSLHEHIANLAGSPAIKKGI | |
| | LQTVKVVDELVKVMGRHKPENIVIEMARENQTTQKGQKNSRERMKRIEEGIKELG | |
| | SQILKEHPVENTQLQNEKLYLYYLQNGRDMYVDQELDINRLSDYDVDHIVPQSFL | |
| | KDDSIDNKVLTRSDKNRGKSDNVPSEEVVKKMKNYWRQLLNAKLITQRKFDNLTK | |
| | AERGGLSELDKAGFIKRQLVETRQITKHVAQILDSRMNTKYDENDKLIREVKVIT | |
| | LKSKLVSDFRKDFQFYKVREINNYHHAHDAYLNAVVGTALIKKYPKLESEFVYGD | |
| | YKVYDVRKMIAKSEQEIGKATAKYFFYSNIMNFFKTEITLANGEIRKRPLIETNG | |
| | ETGEIVWDKGRDFATVRKVLSMPQVNIVKKTEVQTGGFSKESILPKRNSDKLIAR | |
| | KKDWDPKKYGGFDSPTVAYSVLVVAKVEKGKSKKLKSVKELLGITIMERSSFEKN | |
| | PIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANL | |
| | DKVLSAYNKHRD | |

The Cas9 circular permutants that may be useful in the base editor constructs described herein. Exemplary C-terminal fragments of Cas9, based on the Cas9 of SEQ ID NO: 326, which may be rearranged to an N-terminus of Cas9, are provided below. It should be appreciated that such C-terminal fragments of Cas9 are exemplary and are not meant to be limiting. These exemplary CP-Cas9 fragments have the following sequences:

| CP name | Sequence | SEQ ID NO: |
|---|---|---|
| CP1012 C- | DYKVYDVRKMIAKSEQEIGKATAKYFFYSNIMNFFKTEITLANGEIRKRPLIETN | SEQ ID NO: |
| terminal | GETGEIVWDKGRDFATVRKVLSMPQVNIVKKTEVQTGGFSKESILPKRNSDKLIA | 401 |
| fragment | RKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSK | |
| | YVNFLYLASHYEKLKGSPEDNEQKQLFVEQHKHYLDEIIEQISEFSKRVILADAN | |
| | LDKVLSAYNKHRDKPIREQAENIIHLFTLTNLGAPAAFKYFDTTIDRKRYTSTKE | |
| | VLDATLIHQSITGLYETRIDLSQLGGD | |
| CP1028 C- | EIGKATAKYFFYSNIMNFFKTEITLANGEIRKRPLIETNGETGEIVWDKGRDFAT | SEQ ID NO: |
| terminal | VRKVLSMPQVNIVKKTEVQTGGFSKESILPKRNSDKLIARKKDWDPKKYGGFDSP | 402 |
| fragment | TVAYSVLVVAKVEKGKSKKLKSVKELLGITIMERSSFEKNPIDFLEAKGYKEVKK | |
| | DLIIKLPKYSLFELENGRKRMLASAGELQKGNELALPSKYVNFLYLASHYEKLKG | |
| | SPEDNEQKQLFVEQHKHYLDEIIEQISEFSKRVILADANLDKVLSAYNKHRDKPI | |
| | REQAENIIHLFTLTNLGAPAAFKYFDTTIDRKRYTSTKEVLDATLIHQSITGLYE | |
| | TRIDLSQLGGD | |
| CP1041 C- | NIMNFFKTEITLANGEIRKRPLIETNGETGEIVWDKGRDFATVRKVLSMPQVNIV | SEQ ID NO: |
| terminal | KKTEVQTGGFSKESILPKRNSDKLIARKKDWDPKKYGGFDSPTVAYSVLVVAKVE | 403 |
| fragment | KGKSKKLKSVKELLGITIMERSSFEKNPIDFLEAKGYKEVKKDLIIKLPKYSLFE | |
| | LENGRKRMLASAGELQKGNELALPSKYVNFLYLASHYEKLKGSPEDNEQKQLFVE | |
| | QHKHYLDEIIEQISEFSKRVILADANLDKVLSAYNKHRDKPIREQAENIIHLFTL | |
| | TNLGAPAAFKYFDTTIDRKRYTSTKEVLDATLIHQSITGLYETRIDLSQLGGD | |
| CP1249 C- | PEDNEQKQLFVEQHKHYLDEIIEQISEFSKRVILADANLDKVLSAYNKHRDKPIR | SEQ ID NO: |
| terminal | EQAENIIHLFTLTNLGAPAAFKYFDTTIDRKRYTSTKEVLDATLIHQSITGLYET | 404 |
| fragment | RIDLSQLGGD | |
| CP1300 C- | KPIREQAENIIHLFTLTNLGAPAAFKYFDTTIDRKRYTSTKEVLDATLIHQSITG | SEQ ID NO: |
| terminal | LYETRIDLSQLGGD | 405 |
| fragment | | |

In some embodiments, the napDNAbp domain comprises a combination of more than one Cas homolog or variant, such as a circularly permuted Cas variant. In some embodiments, the napDNAbp domain comprises a first Cas variant and a second Cas variant. In some embodiments, the napDNAbp domain comprises a first Cas variant comprising a Cas9-NG and a second Cas variant comprising a Cas9-CP1041 variant. The combination of the CP1041 variant and the NG variant enables both broadened PAM targeting and an expanded editing window. Such a domain is referred to herein as “SpCas9-NG-CP1041.” In some embodiments, the napDNAbp domain comprises an amino acid sequence that has at least 80%, at least 8%, at least 90%, at least 92.5%, at least 95%, at least 97.5%, at least 98%, or at least 99% sequence identity to SEQ ID NO: 463. In some embodiments, the napDNAbp domain comprises the sequence of SEQ ID NO: 463.

(SEQ ID NO: 463)
NIMNFFKTEITLANGEIRKRPLIETNGETGEIVWDKGRDFATVRKVLSMP
QVNIVKKTEVQTGGFSKESIRPKRNSDKLIARKKDWDPKKYGGFVSPTVA
YSVLVVAKVEKGKSKKLKSVKELLGITIMERSSFEKNPIDFLEAKGYKEV
KKDLIIKLPKYSLFELENGRKRMLASARFLQKGNELALPSKYVNFLYLAS
HYEKLKGSPEDNEQKQLFVEQHKHYLDEIIEQISEFSKRVILADANLDKV
LSAYNKHRDKPIREQAENIIHLFTLTNLGAPRAFKYFDTTIDRKVYRSTK
EVLDATLIHQSITGLYETRIDLSQLGGDGGSGGSGGSGGSGGSGGSGGDK
KYSIGLAIGTNSVGWAVITDEYKVPSKKFKVLGNTDRHSIKKNLIGALLF
DSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINA
SGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLS
DILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVG
PLARGNSRFAWMTRKSEETITPWNFEEVVDKGASAQSFIERMTNFDKNLP
NEKVLPKHSLLYEYFTVYNELTKVKYVTEGMRKPAFLSGEQKKAIVDLLF
KTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKR
RRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGR
HKPENIVIEMARENQTTQKGQKNSRERMKRIEEGIKELGSQILKEHPVEN
TQLQNEKLYLYYLQNGRDMYVDQELDINRLSDYDVDHIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREV
KVITLKSKLVSDFRKDFQFYKVREINNYHHAHDAYLNAVVGTALIKKYPK
LESEFVYGDYKVYDVRKMIAKSEQEIGKATAKYFFYS

In some embodiments, the napDNAbp domain comprises a first Cas variant comprising a Cas9-VRQR and a second Cas variant comprising a Cas9-CP1041 variant. Such a domain is referred to herein as “SpCas9-NG-VRQR.” In some embodiments, the napDNAbp domain comprises an amino acid sequence that has at least 80%, at least 8%, at least 90%, at least 92.5%, at least 95%, at least 97.5%, at least 98%, or at least 99% sequence identity to SEQ ID NO: 464. In some embodiments, the napDNAbp domain comprises the sequence of SEQ ID NO: 464.

(SEQ ID NO: 464)
NIMNFFKTEITLANGEIRKRPLIETNGETGEIVWDKGRDFATVRKVLSMP
QVNIVKKTEVQTGGFSKESIRPKRNSDKLIARKKDWDPKKYGGFVSPTVA
YSVLVVAKVEKGKSKKLKSVKELLGITIMERSSFEKNPIDFLEAKGYKEV
KKDLIIKLPKYSLFELENGRKRMLASARFLQKGNELALPSKYVNFLYLAS
HYEKLKGSPEDNEQKQLFVEQHKHYLDEIIEQISEFSKRVILADANLDKV
LSAYNKHRDKPIREQAENIIHLFTLTNLGAPRAFKYFDTTIDRKVYRSTK
EVLDATLIHQSITGLYETRIDLSQLGGDGGSGGSGGSGGSGGSGGSGGDK
KYSIGLAIGTNSVGWAVITDEYKVPSKKFKVLGNTDRHSIKKNLIGALLF
DSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINA
SGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLS
DILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVG
PLARGNSRFAWMTRKSEETITPWNFEEVVDKGASAQSFIERMTNFDKNLP
NEKVLPKHSLLYEYFTVYNELTKVKYVTEGMRKPAFLSGEQKKAIVDLLF
KTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKR
RRYTGWGRLSRKLINGIRDKQSGKTILDFLKSDGFANRNFMQLIHDDSLT
FKEDIQKAQVSGQGDSLHEHIANLAGSPAIKKGILQTVKVVDELVKVMGR
HKPENIVIEMARENQTTQKGQKNSRERMKRIEEGIKELGSQILKEHPVEN
TQLQNEKLYLYYLQNGRDMYVDQELDINRLSDYDVDHIVPQSFLKDDSID
NKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREV
KVITLKSKLVSDFRKDFQFYKVREINNYHHAHDAYLNAVVGTALIKKYPK
LESEFVYGDYKVYDVRKMIAKSEQEIGKATAKYFFYS

I. Cas9 Variants with Modified PAM Specificities

The adenine base editors of the present disclosure may also comprise Cas9 variants with modified PAM specificities. Some aspects of this disclosure provide Cas9 proteins that exhibit activity on a target sequence that does not comprise the canonical PAM (5′-NGG-3′, where N is A, C, G, or T) at its 3′-end. In some embodiments, the Cas9 protein exhibits activity on a target sequence comprising a 5′-NGG-3′ PAM sequence at its 3′-end. In some embodiments, the Cas9 protein exhibits activity on a target sequence comprising a 5′-NNG-3′ PAM sequence at its 3′-end. In some embodiments, the Cas9 protein exhibits activity on a target sequence comprising a 5′-NNA-3′ PAM sequence at its 3′-end. In some embodiments, the Cas9 protein exhibits activity on a target sequence comprising a 5′-NNC-3′ PAM sequence at its 3′-end. In some embodiments, the Cas9 protein exhibits activity on a target sequence comprising a 5′-NNT-3′ PAM sequence at its 3′-end. In some embodiments, the Cas9 protein exhibits activity on a target sequence comprising a 5′-NGT-3′ PAM sequence at its 3′-end. In some embodiments, the Cas9 protein exhibits activity on a target sequence comprising a 5′-NGA-3′ PAM sequence at its 3′-end. In some embodiments, the Cas9 protein exhibits activity on a target sequence comprising a 5′-NGC-3′ PAM sequence at its 3′-end. In some embodiments, the Cas9 protein exhibits activity on a target sequence comprising a 5′-NAA-3′ PAM sequence at its 3′-end. In some embodiments, the Cas9 protein exhibits activity on a target sequence comprising a 5′-NAC-3′ PAM sequence at its 3′-end. In some embodiments, the Cas9 protein exhibits activity on a target sequence comprising a 5′-NAT-3′ PAM sequence at its 3′-end. In still other embodiments, the Cas9 protein exhibits activity on a target sequence comprising a 5′-NAG-3′ PAM sequence at its 3′-end.

In some embodiments, the disclosed adenine base editors comprise a napDNAbp domain comprising a SpCas9-NG, which has a PAM that corresponds to NGN. In some embodiments, the disclosed base editors comprise a napDNAbp domain that has a sequence that is at least 90%, at least 95%, at least 98%, or at least 99% identical to SpCas9-NG. The sequence of SpCas9-NG is illustrated below:

(SEQ ID NO: 477)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESIRPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASARFLQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPRAFKYFDTTIDRKVYRSTKEVLDATLIHQ
SITGLYETRIDLSQLGGD

In some embodiments, the disclosed base editors comprise a napDNAbp domain comprising a S. aureus Cas9 nickase KKH, or SaCas9-KKH, which has a PAM that corresponds to NNNRRT. This Cas9 variant contains the amino acid substitutions D10A, E782K, N968K, and R1015H relative to wild-type SaCas9, set forth as SEQ ID NO: 377. In some embodiments, the disclosed base editors comprise a napDNAbp domain that has a sequence that is at least 90%, at least 95%, at least 98%, or at least 99% identical to SaCas9-KKH. The sequence of SaCas9-KKH is illustrated below: S. aureus Cas9 nickase KKH (SaCas9-KKH)

(SEQ ID NO: 478)
MGKRNYILGLAIGITSVGYGIIDYETRDVIDAGVRLFKEANVENNEGRRS
KRGARRLKRRRRHRIQRVKKLLFDYNLLTDHSELSGINPYEARVKGLSQK
LSEEEFSAALLHLAKRRGVHNVNEVEEDTGNELSTKEQISRNSKALEEKY
VAELQLERLKKDGEVRGSINRFKTSDYVKEAKQLLKVQKAYHQLDQSFID
TYIDLLETRRTYYEGPGEGSPFGWKDIKEWYEMLMGHCTYFPEELRSVKY
AYNADLYNALNDLNNLVITRDENEKLEYYEKFQIIENVFKQKKKPTLKQI
AKEILVNEEDIKGYRVTSTGKPEFTNLKVYHDIKDITARKEIIENAELLD
QIAKILTIYQSSEDIQEELTNLNSELTQEEIEQISNLKGYTGTHNLSLKA
INLILDELWHTNDNQIAIFNRLKLVPKKVDLSQQKEIPTTLVDDFILSPV
VKRSFIQSIKVINAIIKKYGLPNDIIIELAREKNSKDAQKMINEMQKRNR
QTNERIEEIIRTTGKENAKYLIEKIKLHDMQEGKCLYSLEAIPLEDLLNN
PFNYEVDHIIPRSVSFDNSFNNKVLVKQEENSKKGNRTPFQYLSSSDSKI
SYETFKKHILNLAKGKGRISKTKKEYLLEERDINRFSVQKDFINRNLVDT
RYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQE
YKEIFITPHQIKHIKDFKDYKYSHRVDKKPNRKLINDTLYSTRKDDKGNT
LIVNNLNGLYDKDNDKLKKLINKSPEKLLMYHHDPQTYQKLKLIMEQYGD
EKNPLYKYYEETGNYLTKYSKKDNGPVIKKIKYYGNKLNAHLDITDDYPN
SRNKVVKLSLKPYRFDVYLDNGVYKFVTVKNLDVIKKENYYEVNSKCYEE
AKKLKKISNQAEFIASFYKNDLIKINGELYRVIGVNNDLLNRIEVNMIDI
TYREYLENMNDKRPPHIIKTIASKTQSIKKYSTDILGNLYEVKSKKHPQI
IKKG

In some embodiments, the disclosed adenine base editors comprise a napDNAbp domain comprising a S. pyogenes Cas9 nickase KKH, or SpCas9-KKH, which has a PAM that corresponds to NNNRRT.

In some embodiments, the disclosed adenine base editors comprise a napDNAbp domain comprising a xCas9, an evolved variant of SpCas9. In some embodiments, the disclosed base editors comprise a napDNAbp domain that has a sequence that is at least 90%, at least 95%, at least 98%, or at least 99% identical to xCas9. The sequence of xCas9 is illustrated below:

(SEQ ID NO: 479)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDTKLQLSKDTYDDDLDNLLAQIGDQYADLFLAAKNLSDAI
LLSDILRVNTEITKAPLSASMIKLYDEHHQDLTLLKALVRQQLPEKYKEI
FFDQSKNGYAGYIDGGASQEEFYKFIKPILEKMDGTEELLVKLNREDLLR
KQRTFDNGIIPHQIHLGELHAILRRQEDFYPFLKDNREKIEKILTFRIPY
YVGPLARGNSRFAWMTRKSEETITPWNFEKVVDKGASAQSFIERMTNFDK
NLPNEKVLPKHSLLYEYFTVYNELTKVKYVTEGMRKPAFLSGDQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFIQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGVLQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

It should be appreciated that any of the amino acid mutations described herein, (e.g., A262T) from a first amino acid residue (e.g., A) to a second amino acid residue (e.g., T) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine (e.g., a A262T mutation) may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine. The skilled artisan would recognize that such conservative amino acid substitutions will likely have minor effects on protein structure and are likely to be well tolerated without compromising function. In some embodiments, any amino of the amino acid mutations provided herein from one amino acid to a threonine may be an amino acid mutation to a serine. In some embodiments, any amino of the amino acid mutations provided herein from one amino acid to an arginine may be an amino acid mutation to a lysine. In some embodiments, any amino of the amino acid mutations provided herein from one amino acid to an isoleucine, may be an amino acid mutation to an alanine, valine, methionine, or leucine. In some embodiments, any amino of the amino acid mutations provided herein from one amino acid to a lysine may be an amino acid mutation to an arginine. In some embodiments, any amino of the amino acid mutations provided herein from one amino acid to an aspartic acid may be an amino acid mutation to a glutamic acid or asparagine. In some embodiments, any amino of the amino acid mutations provided herein from one amino acid to a valine may be an amino acid mutation to an alanine, isoleucine, methionine, or leucine. In some embodiments, any amino of the amino acid mutations provided herein from one amino acid to a glycine may be an amino acid mutation to an alanine. It should be appreciated, however, that additional conserved amino acid residues would be recognized by the skilled artisan and any of the amino acid mutations to other conserved amino acid residues are also within the scope of this disclosure.

In some embodiments, the present disclosure may utilize any of the Cas9 variants disclosed in the SEQUENCES section below.

In some embodiments, the Cas9 protein comprises a combination of mutations that exhibit activity on a target sequence comprising a 5′-NAA-3 PAM sequence at its 3′-end. In some embodiments, the combination of mutations is present in any one of the clones listed in Table 1. In some embodiments, the combination of mutations is conservative mutations of the clones listed in Table 1. In some embodiments, the Cas9 protein comprises the combination of mutations of any one of the Cas9 clones listed in Table 1.

TABLE 1
NAA PAM Clones
Mutations from wild-type SpCas9 (e.g., SEQ ID NO: 326)

D177N, K218R, D614N, D1135N, P1137S, E1219V, A1320V, A1323D, R1333K
D177N, K218R, D614N, D1135N, E1219V, Q1221H, H1264Y, A1320V, R1333K
A10T, I322V, S409I, E427G, G715C, D1135N, E1219V, Q1221H, H1264Y, A1320V, R1333K
A367T, K710E, R1114G, D1135N, P1137S, E1219V, Q1221H, H1264Y, A1320V, R1333K
A10T, I322V, S409I, E427G, R753G, D861N, D1135N, K1188R, E1219V, Q1221H, H1264H,
A1320V, R1333K
A10T, I322V, S409I, E427G, R654L, V743I, R753G, M1021T, D1135N, D1180G, K1211R,
E1219V, Q1221H, H1264Y, A1320V, R1333K
A10T, I322V, S409I, E427G, V743I, R753G, E762G, D1135N, D1180G, K1211R, E1219V,
Q1221H, H1264Y, A1320V, R1333K
A10T, I322V, S409I, E427G, R753G, D1135N, D1180G, K1211R, E1219V, Q1221H, H1264Y,
S1274R, A1320V, R1333K
A10T, I322V, S409I, E427G, A589S, R753G, D1135N, E1219V, Q1221H, H1264H, A1320V,
R1333K
A10T, I322V, S409I, E427G, R753G, E757K, G865G, D1135N, E1219V, Q1221H, H1264Y,
A1320V, R1333K
A10T, I322V, S409I, E427G, R654L, R753G, E757K, D1135N, E1219V, Q1221H, H1264Y,
A1320V, R1333K
A10T, I322V, S409I, E427G, K599R, M631A, R654L, K673E, V743I, R753G, N758H, E762G,
D1135N, D1180G, E1219V, Q1221H, Q1256R, H1264Y, A1320V, A1323D, R1333K
A10T, I322V, S409I, E427G, R654L, K673E, V743I, R753G, E762G, N869S, N1054D, R1114G,
D1135N, D1180G, E1219V, Q1221H, H1264Y, A1320V, A1323D, R1333K
A10T, I322V, S409I, E427G, R654L, L727I, V743I, R753G, E762G, R859S, N946D, F1134L,
D1135N, D1180G, E1219V, Q1221H, H1264Y, N1317T, A1320V, A1323D, R1333K
A10T, I322V, S409I, E427G, R654L, K673E, V743I, R753G, E762G, N803S, N869S, Y1016D,
G1077D, R1114G, F1134L, D1135N, D1180G, E1219V, Q1221H, H1264Y, V1290G, L1318S,
A1320V, A1323D, R1333K
A10T, I322V, S409I, E427G, R654L, K673E, V743I, R753G, E762G, N803S, N869S, Y1016D,
G1077D, R1114G, F1134L, D1135N, K1151E, D1180G, E1219V, Q1221H, H1264Y, V1290G,
L1318S, A1320V, R1333K
A10T, I322V, S409I, E427G, R654L, K673E, V743I, R753G, E762G, N803S, N869S, Y1016D,
G1077D, R1114G, F1134L, D1135N, D1180G, E1219V, Q1221H, H1264Y, V1290G, L1318S,
A1320V, A1323D, R1333K
A10T, I322V, S409I, E427G, R654L, K673E, F693L, V743I, R753G, E762G, N803S, N869S,
L921P, Y1016D, G1077D, F1080S, R1114G, D1135N, D1180G, E1219V, Q1221H, H1264Y,
L1318S, A1320V, A1323D, R1333K
A10T, I322V, S409I, E427G, E630K, R654L, K673E, V743I, R753G, E762G, Q768H, N803S,
N869S, Y1016D, G1077D, R1114G, F1134L, D1135N, D1180G, E1219V, Q1221H, H1264Y,
L1318S, A1320V, R1333K
A10T, I322V, S409I, E427G, R654L, K673E, F693L, V743I, R753G, E762G, Q768H, N803S,
N869S, Y1016D, G1077D, R1114G, F1134L, D1135N, D1180G, E1219V, Q1221H, G1223S,
H1264Y, L1318S, A1320V, R1333K
A10T, I322V, S409I, E427G, R654L, K673E, F693L, V743I, R753G, E762G, N803S, N869S,
L921P, Y1016D, G1077D, F1801S, R1114G, D1135N, D1180G, E1219V, Q1221H, H1264Y,
L1318S, A1320V, A1323D, R1333K
A10T, I322V, S409I, E427G, R654L, V743I, R753G, M1021T, D1135N, D1180G, K1211R,
E1219V, Q1221H, H1264Y, A1320V, R1333K
A10T, I322V, S409I, E427G, R654L, K673E, V743I, R753G, E762G, M673I, N803S, N869S,
G1077D, R1114G, D1135N, V1139A, D1180G, E1219V, Q1221H, A1320V, R1333K
A10T, I322V, S409I, E427G, R654L, K673E, V743I, R753G, E762G, N803S, N869S, R1114G,
D1135N, E1219V, Q1221H, A1320V, R1333K

In some embodiments, the Cas9 protein comprises an amino acid sequence that is at least 80% identical to the amino acid sequence of a Cas9 protein as provided by any one of the variants of Table 1. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 1.

In some embodiments, the Cas9 protein exhibits an increased activity on a target sequence that does not comprise the canonical PAM (5′-NGG-3′) at its 3′ end as compared to Streptococcus pyogenes Cas9 as provided by SEQ ID NO: 326. In some embodiments, the Cas9 protein exhibits an activity on a target sequence having a 3′ end that is not directly adjacent to the canonical PAM sequence (5′-NGG-3′) that is at least 5-fold increased as compared to the activity of Streptococcus pyogenes Cas9 as provided by SEQ ID NO: 326 on the same target sequence. In some embodiments, the Cas9 protein exhibits an activity on a target sequence that is not directly adjacent to the canonical PAM sequence (5′-NGG-3′) that is at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes as provided by SEQ ID NO: 326 on the same target sequence. In some embodiments, the 3′ end of the target sequence is directly adjacent to an AAA, GAA, CAA, or TAA sequence. In some embodiments, the Cas9 protein comprises a combination of mutations that exhibit activity on a target sequence comprising a 5′-NAC-3″ PAM sequence at its 3′-end. In some embodiments, the combination of mutations is present in any one of the clones listed in Table 2. In some embodiments, the combination of mutations is conservative mutations of the clones listed in Table 2. In some embodiments, the Cas9 protein comprises the combination of mutations of any one of the Cas9 clones listed in Table 2.

TABLE 2
NAC PAM Clones
MUTATIONS FROM WILD-TYPE SPCAS9 (E.G., SEQ ID NO: 326)

T472I, R753G, K890E, D1332N, R1335Q, T1337N
I1057S, D1135N, P1301S, R1335Q, T1337N
T472I, R753G, D1332N, R1335Q, T1337N
D1135N, E1219V, D1332N, R1335Q, T1337N
T472I, R753G, K890E, D1332N, R1335Q, T1337N
I1057S, D1135N, P1301S, R1335Q, T1337N
T472I, R753G, D1332N, R1335Q, T1337N
T472I, R753G, Q771H, D1332N, R1335Q, T1337N
E627K, T638P, K652T, R753G, N803S, K959N, R1114G, D1135N, E1219V, D1332N, R1335Q,
T1337N
E627K, T638P, K652T, R753G, N803S, K959N, R1114G, D1135N, K1156E, E1219V, D1332N,
R1335Q, T1337N
E627K, T638P, V647I, R753G, N803S, K959N, G1030R, I1055E, R1114G, D1135N, E1219V,
D1332N, R1335Q, T1337N
E627K, E630G, T638P, V647A, G687R, N767D, N803S, K959N, R1114G, D1135N, E1219V,
D1332G, R1335Q, T1337N
E627K, T638P, R753G, N803S, K959N, R1114G, D1135N, E1219V, N1266H, D1332N, R1335Q,
T1337N
E627K, T638P, R753G, N803S, K959N, I1057T, R1114G, D1135N, E1219V, D1332N, R1335Q,
T1337N
E627K, T638P, R753G, N803S, K959N, R1114G, D1135N, E1219V, D1332N, R1335Q, T1337N
E627K, M631I, T638P, R753G, N803S, K959N, Y1036H, R1114G, D1135N, E1219V, D1251G,
D1332G, R1335Q, T1337N
E627K, T638P, R753G, N803S, V875I, K959N, Y1016C, R1114G, D1135N, E1219V, D1251G,
D1332G, R1335Q, T1337N, I1348V
K608R, E627K, T638P, V647I, R654L, R753G, N803S, T804A, K848N, V922A, K959N, R1114G,
D1135N, E1219V, D1332N, R1335Q, T1337N
K608R, E627K, T638P, V647I, R753G, N803S, V922A, K959N, K1014N, V1015A, R1114G,
D1135N, K1156N, E1219V, N1252D, D1332N, R1335Q, T1337N
K608R, E627K, R629G, T638P, V647I, A711T, R753G, K775R, K789E, N803S, K959N, V1015A,
Y1036H, R1114G, D1135N, E1219V, N1286H, D1332N, R1335Q, T1337N
K608R, E627K, T638P, V647I, T740A, R753G, N803S, K948E, K959N, Y1016S, R1114G,
D1135N, E1219V, N1286H, D1332N, R1335Q, T1337N
K608R, E627K, T638P, V647I, T740A, N803S, K948E, K959N, Y1016S, R1114G, D1135N,
E1219V, N1286H, D1332N, R1335Q, T1337N
I670S, K608R, E627K, E630G, T638P, V647I, R653K, R753G, I795L, K797N, N803S, K866R,
K890N, K959N, Y1016C, R1114G, D1135N, E1219V, D1332N, R1335Q, T1337N
K608R, E627K, T638P, V647I, T740A, G752R, R753G, K797N, N803S, K948E, K959N, V1015A,
Y1016S, R1114G, D1135N, E1219V, N1266H, D1332N, R1335Q, T1337N
I570T, A589V, K608R, E627K, T638P, V647I, R654L, Q716R, R753G, N803S, K948E, K959N,
Y1016S, R1114G, D1135N, E1207G, E1219V, N1234D, D1332N, R1335Q, T1337N
K608R, E627K, R629G, T638P, V647I, R654L, Q740R, R753G, N803S, K959N, N990S, T995S,
V1015A, Y1036D, R1114G, D1135N, E1207G, E1219V, N1234D, N1266H, D1332N, R1335Q,
T1337N
I562F, V565D, I570T, K608R, L625S, E627K, T638P, V647I, R654I, G752R, R753G, N803S,
N808D, K959N, M1021L, R1114G, D1135N, N1177S, N1234D, D1332N, R1335Q, T1337N
I562F, I570T, K608R, E627K, T638P, V647I, R753G, E790A, N803S, K959N, V1015A, Y1036H,
R1114G, D1135N, D1180E, A1184T, E1219V, D1332N, R1335Q, T1337N
I570T, K608R, E627K, T638P, V647I, R654H, R753G, E790A, N803S, K959N, V1015A, R1114G,
D1127A, D1135N, E1219V, D1332N, R1335Q, T1337N
I570T, K608R, L625S, E627K, T638P, V647I, R654I, T703P, R753G, N803S, N808D, K959N,
M1021L, R1114G, D1135N, E1219V, D1332N, R1335Q, T1337N
I570S, K608R, E627K, E630G, T638P, V647I, R653K, R753G, I795L, N803S, K866R, K890N,
K959N, Y1016C, R1114G, D1135N, E1219V, D1332N, R1335Q, T1337N
I570T, K608R, E627K, T638P, V647I, R654H, R753G, E790A, N803S, K959N, V1016A, R1114G,
D1135N, E1219V, K1246E, D1332N, R1335Q, T1337N
K608R, E627K, T638P, V647I, R654L, K673E, R753G, E790A, N803S, K948E, K959N, R1114G,
D1127G, D1135N, D1180E, E1219V, N1286H, D1332N, R1335Q, T1337N
K608R, L625S, E627K, T638P, V647I, R654I, I670T, R753G, N803S, N808D, K959N, M1021L,
R1114G, D1135N, E1219V, N1286H, D1332N, R1335Q, T1337N
E627K, M631V, T638P, V647I, K710E, R753G, N803S, N808D, K948E, M1021L, R1114G,
D1135N, E1219V, D1332N, R1335Q, T1337N, S1338T, H1349R

In some embodiments, the Cas9 protein comprises an amino acid sequence that is at least 80% identical to the amino acid sequence of a Cas9 protein as provided by any one of the variants of Table 2. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the variants of Table 2.

In some embodiments, the Cas9 protein comprises a combination of mutations that exhibit activity on a target sequence comprising a 5 ‘-NAT-3’ PAM sequence at its 3′-end. In some embodiments, the combination of mutations is present in any one of the clones listed in Table 3. In some embodiments, the combination of mutations is conservative mutations of the clones listed in Table 3. In some embodiments, the Cas9 protein comprises the combination of mutations of any one of the Cas9 clones listed in Table 3.

TABLE 3
NAT PAM Clones
MUTATIONS FROM WILD-TYPE SPCAS9 (E.G., SEQ ID NO: 326)

K961E, H985Y, D1135N, K1191N, E1219V, Q1221H, A1320A, P1321S, R1335L
D1135N, G1218S, E1219V, Q1221H, P1249S, P1321S, D1322G, R1335L
V743I, R753G, E790A, D1135N, G1218S, E1219V, Q1221H, A1227V, P1249S, N1286K, A1293T,
P1321S, D1322G, R1335L, T1339I
F575S, M631L, R654L, V748I, V743I, R753G, D853E, V922A, R1114G D1135N, G1218S,
E1219V, Q1221H, A1227V, P1249S, N1286K, A1293T, P1321S, D1322G, R1335L, T1339I
F575S, M631L, R654L, R664K, R753G, D853E, V922A, R1114G D1135N, D1180G, G1218S,
E1219V, Q1221H, P1249S, N1286K, P1321S, D1322G, R1335L
M631L, R654L, R753G, K797E, D853E, V922A, D1012A, R1114G D1135N, G1218S, E1219V,
Q1221H, P1249S, N1317K, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, D596Y, M631L, R654L, R664K, R753G, D853E, V922A, R1114G, Y1131C, D1135N,
D1180G, G1218S, E1219V, Q1221H, P1249S, Q1256R, P1321S, D1322G, R1335L
F575S, M631L, R654L, R664K, K710E, V750A, R753G, D853E, V922A, R1114G, Y1131C,
D1135N, D1180G, G1218S, E1219V, Q1221H, P1249S, P1321S, D1322G, R1335L
F575S, M631L, K649R, R654L, R664K, R753G, D853E, V922A, R1114G, Y1131C, D1135N,
K1156E, D1180G, G1218S, E1219V, Q1221H, P1249S, P1321S, D1322G, R1335L
F575S, M631L, R654L, R664K, R753G, D853E, V922A, R1114G, Y1131C, D1135N, D1180G,
G1218S, E1219V, Q1221H, P1249S, P1321S, D1322G, R1335L
F575S, M631L, R654L, R664K, R753G, D853E, V922A, I1057G, R1114G, Y1131C, D1135N,
D1180G, G1218S, E1219V, Q1221H, P1249S, N1308D, P1321S, D1322G, R1335L
M631L, R654L, R753G, D853E, V922A, R1114G, Y1131C, D1135N, E1150V, D1180G, G1218S,
E1219V, Q1221H, P1249S, P1321S, D1332G, R1335L
M631L, R654L, R664K, R753G, D853E, I1057V, Y1131C, D1135N, D1180G, G1218S, E1219V,
Q1221H, P1249S, P1321S, D1332G, R1335L
M631L, R654L, R664K, R753G, I1057V, R1114G, Y1131C, D1135N, D1180G, G1218S, E1219V,
Q1221H, P1249S, P1321S, D1332G, R1335L

The above description of various napDNAbps which can be used in connection with the presently disclose adenine base editors is not meant to be limiting in any way. The adenine base editors may comprise the canonical SpCas9, or any ortholog Cas9 protein, or any variant Cas9 protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave of strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats). The adenine base editors described herein may also comprise Cas9 equivalents, including Cas12a/Cpf1 and Cas12b proteins which are the result of convergent evolution. The napDNAbps used herein (e.g., SpCas9, Cas9 variant, or Cas9 equivalents) may also may also contain various modifications that alter/enhance their PAM specifities. Lastly, the application contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a references SpCas9 canonical sequences or a reference Cas9 equivalent (e.g., Cas12a/Cpf1).

In a particular embodiment, the Cas9 variant having expanded PAM capabilities is SpCas9 (H840A) VRQR, or SpCas9-VRQR. In some embodiments, the disclosed base editors comprise a napDNAbp domain that has a sequence that is at least 90%, at least 95%, at least 98%, or at least 99% identical to SpCas9-VRQR. In some embodiments, the disclosed base editors comprise a napDNAbp domain that comprises SpCas9-VRQR. The SpCas9-VRQR comprises the following amino acid sequence (with the V, R, Q, R substitutions relative to the SpCas9 (H840A) of SEQ ID NO: 370 show, in bold underline. In addition, the methionine residue in SpCas9 (H840) was removed for SpCas9 (H840A) VRQR):

(SEQ ID NO: 406)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKQYRSTKEVLDATLIHQS
ITGLYETRIDLSQLGGD

In another particular embodiment, the Cas9 variant having expanded PAM capabilities is SpCas9 (H840A) VRER, having the following amino acid sequence (with the V, R, E, R substitutions relative to the SpCas9 (H840A) of SEQ ID NO: 370 are shown in bold underline. In addition, the methionine residue in SpCas9 (H840) was removed for SpCas9 (H840A) VRER):

(SEQ ID NO: 407)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKEYRSTKEVLDATLIHQS
ITGLYETRIDLSQLGGD

In another particular embodiment, the Cas9 variant having expanded PAM capabilities is SpCas9 (H840A) VQR, having the D10A, D1135V, R1335Q, and T1337R substitutions relative to the SpCas9 (H840A) of SEQ ID NO: 370. In addition, the methionine residue in SpCas9 (H840) was removed for SpCas9 (H840A) VQR):

(SEQ ID NO: 480)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKQYRSTKEVLDATLIHQ
SITGLYETRIDLSQLGGD

In another particular embodiment, the Cas9 variant having expanded PAM capabilities is SpCas9 (H840A) EQR, having the D10A, D1135E, R1335Q, and T1337R substitutions relative to the SpCas9 (H840A) of SEQ ID NO: 370. In addition, the methionine residue in SpCas9 (H840) was removed for SpCas9 (H840A) EQR):

(SEQ ID NO: 481)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFESPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKQYRSTKEVLDATLIHQ
SITGLYETRIDLSQLGGD

In addition, any available methods may be utilized to obtain or construct a variant or mutant Cas9 protein. The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Because of their nature, gain-of-function mutations are usually dominant.

Mutations can be introduced into a reference Cas9 protein using site-directed mutagenesis. Older methods of site-directed mutagenesis known in the art rely on sub-cloning of the sequence to be mutated into a vector, such as an M13 bacteriophage vector, that allows the isolation of single-stranded DNA template. In these methods, one anneals a mutagenic primer (i.e., a primer capable of annealing to the site to be mutated but bearing one or more mismatched nucleotides at the site to be mutated) to the single-stranded template and then polymerizes the complement of the template starting from the 3′ end of the mutagenic primer. The resulting duplexes are then transformed into host bacteria and plaques are screened for the desired mutation. More recently, site-directed mutagenesis has employed PCR methodologies, which have the advantage of not requiring a single-stranded template. In addition, methods have been developed that do not require sub-cloning. Several issues must be considered when PCR-based site-directed mutagenesis is performed. First, in these methods it is desirable to reduce the number of PCR cycles to prevent expansion of undesired mutations introduced by the polymerase. Second, a selection must be employed in order to reduce the number of non-mutated parental molecules persisting in the reaction. Third, an extended-length PCR method is preferred in order to allow the use of a single PCR primer set. And fourth, because of the non-template-dependent terminal extension activity of some thermostable polymerases it is often necessary to incorporate an end-polishing step into the procedure prior to blunt-end ligation of the PCR-generated mutant product.

Any of the references noted above which relate to napDNAbp domains are hereby incorporated by reference in their entireties, if not already stated so.

### Base Editor Architectures Comprising a Nuclease Programmable DNA Binding Protein and an Adenosine Deaminase Domain

In some aspects, the disclosure provide base editors comprising a napDNAbp domain and an adenosine deaminase domain as described herein. The Cas9 domain may be any of the Cas9 domains or Cas9 proteins (e.g., a nCas9) provided herein. In some embodiments, any of the Cas9 domains or Cas9 proteins (e.g., nCas9) provided herein may be fused with any of the adenosine deaminases provided herein.

In some embodiments, the base editors comprising adenosine deaminases and a napDNAbp (e.g., Cas9 domain) do not include a linker sequence. In some embodiments, a linker is present between the adenosine deaminase domain and/or between an adenosine deaminase and the napDNAbp. In some embodiments, the “14” used in the general architecture above indicates the presence of an optional linker. In some embodiments, an adenosine deaminase domain and the napDNAbp domain are fused via any of the linkers provided herein. For example, in some embodiments the adenosine deaminase domain (which may include one or more adenosine deaminases) and the napDNAbp are fused via any of the linkers provided below in the section entitled “Linkers”. In certain embodiments, the base editors comprise an ABE7.10 (or ABEmax) architecture, which comprises NH₂—[NLS]-[first adenosine deaminase]-[second adenosine deaminase]-[napDNAbp domain]-[NLS]—COOH. In certain embodiments, the base editors comprise an ABE7.10 monomer architecture, which comprises NH₂—[NLS]-[adenosine deaminase]-[napDNAbp domain]-[NLS]—COOH.

In some embodiments, the base editors provided herein further comprise one or more nuclear targeting sequences, for example, a nuclear localization sequence (NLS). In some embodiments, a NLS comprises an amino acid sequence that facilitates the importation of a protein, that comprises an NLS, into the cell nucleus (e.g., by nuclear transport). In some embodiments, any of the base editors provided herein further comprise one or more nuclear localization sequences (NLSs). In certain embodiments, any of the base editors comprise two NLSs. In some embodiments, one or more of the NLSs are bipartite NLSs (“bpNLS”). In certain embodiments, the disclosed base editors comprise two bipartite NLSs. In some embodiments, the disclosed base editors comprise more than two bipartite NLSs.

In some embodiments, the NLS is fused to the N-terminus of the base editor. In some embodiments, the NLS is fused to the C-terminus of the base editor. In some embodiments, the NLS is fused to the C-terminus of the napDNAbp. In some embodiments, the NLS is fused to the N-terminus of the adenosine deaminase. In some embodiments, the NLS is fused to the C-terminus of the adenosine deaminase. In some embodiments, the NLS is fused to the base editor via one or more linkers. In some embodiments, the NLS is fused to the base editor without a linker.

In some embodiments, the NLS comprises an amino acid sequence of any one of the NLS sequences provided or referenced herein. In some embodiments, the NLS comprises an amino acid sequence as set forth in SEQ ID NO: 408 or SEQ ID NO: 409. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, a NLS comprises the amino acid sequence PKKKRKV (SEQ ID NO: 408), MDSLLMNRRKFLYQFKNVRWAKGRRETYLC (SEQ ID NO: 409), KRTADGSEFESPKKKRKV (SEQ ID NO: 410), or KRTADGSEFEPKKKRKV (SEQ ID NO: 411). In other embodiments, the NLS comprises the amino acid sequence:

(SEQ ID NO: 482)
NLSKRPAAIKKAGQAKKKK,
(SEQ ID NO: 483)
PAAKRVKLD,
(SEQ ID NO: 484)
RQRRNELKRSF,
or
(SEQ ID NO: 485)
NQSSNFGPMKGGNFGGRSSGPYGGGGQYFAKPRNQGGY.

In some embodiments, the base editors provided herein do not comprise a linker. In some embodiments, a linker is present between one or more of the domains or proteins (e.g., adenosine deaminase, napDNAbp, and/or NLS). In some embodiments, the “]-[” used in the general architecture above indicates the presence of an optional linker.

In some embodiments, the general architecture of exemplary base editors with a first adenosine deaminase, a second adenosine deaminase, and a napDNAbp domain comprises any one of the following structures, where NLS is a nuclear localization sequence (e.g., any NLS provided herein), NH₂ is the N-terminus of the base editor, and COOH is the C-terminus of the base editor.

In some embodiments, the general architecture of exemplary base editors comprising an adenosine deaminase domain and a napDNAbp: NH₂-[adenosine deaminase]-[napDNAbp domain]-COOH; or NH₂-[napDNAbp domain]-[adenosine deaminase]-COOH.

In some embodiments, the architecture of exemplary base editors comprise an adenosine deaminase domain that comprises a dimer of a first adenosine deaminase and a second adenosine deaminase: NH₂-[first adenosine deaminase]-[second adenosine deaminase]-[napDNAbp domain]-COOH; NH₂-[first adenosine deaminase]-[napDNAbp domain]-[second adenosine deaminase]-COOH; NH₂-[napDNAbp domain]-[first adenosine deaminase]-[second adenosine deaminase]-COOH; NH₂-[second adenosine deaminase]-[first adenosine deaminase]-[napDNAbp domain]-COOH; NH₂-[second adenosine deaminase]-[napDNAbp domain]-[first adenosine deaminase]-COOH; or NH₂-[napDNAbp domain]-[second adenosine deaminase]-[first adenosine deaminase]-COOH.

In particular embodiments, the disclosure provides a base editor comprising the architecture NH₂-[first adenosine deaminase]-[second adenosine deaminase]-[napDNAbp domain]-[NLS]—COOH.

Exemplary base editors comprising an adenosine deaminase, a napDNAbp domain, and an NLS, where NLS is a nuclear localization sequence (e.g., any NLS provided herein) may have the following architecture:
NH₂-[adenosine deaminase]-[napDNAbp domain]-[NLS]—COOH;
NH₂-[napDNAbp domain]-[adenosine deaminase]-[NLS]—COOH;
NH₂—[NLS]-[adenosine deaminase]-[napDNAbp domain]-COOH; or
NH₂—[NLS]-[napDNAbp domain]-[adenosine deaminase]-COOH.

Exemplary base editors comprising a first adenosine deaminase, a second adenosine deaminase, a napDNAbp domain, and an NLS, where NLS is a nuclear localization sequence (e.g., any NLS provided herein) may have the following architecture: NH₂—[NLS]-[first adenosine deaminase]-[second adenosine deaminase]-[napDNAbp domain]-COOH;
NH₂-[first adenosine deaminase]-[NLS]-[second adenosine deaminase]-[napDNAbp domain]-COOH;
NH₂-[first adenosine deaminase]-[second adenosine deaminase]-[NLS]-[napDNAbp domain]-COOH;
NH₂-[first adenosine deaminase]-[second adenosine deaminase]-[napDNAbp domain]-[NLS]—COOH;
NH₂— [NLS]-[first adenosine deaminase]-[napDNAbp domain]-[second adenosine deaminase]-COOH;
NH₂-[first adenosine deaminase]-[NLS]-[napDNAbp domain]-[second adenosine deaminase]-COOH;
NH₂-[first adenosine deaminase]-[napDNAbp domain]-[NLS]-[second adenosine deaminase]-COOH;
NH₂-[first adenosine deaminase]-[napDNAbp domain]-[second adenosine deaminase]-[NLS]—COOH;
NH₂—[NLS]-[napDNAbp domain]-[first adenosine deaminase]-[second adenosine deaminase]-COOH;
NH₂-[napDNAbp domain]-[NLS]-[first adenosine deaminase]-[second adenosine deaminase]-COOH;
NH₂-[napDNAbp domain]-[first adenosine deaminase]-[NLS]-[second adenosine deaminase]-COOH;
NH₂-[napDNAbp domain]-[first adenosine deaminase]-[second adenosine deaminase]-[NLS]—COOH;
NH₂—[NLS]-[second adenosine deaminase]-[first adenosine deaminase]-[napDNAbp domain]-COOH;
NH₂-[second adenosine deaminase]-[NLS]-[first adenosine deaminase]-[napDNAbp domain]-COOH;
NH₂-[second adenosine deaminase]-[first adenosine deaminase]-[NLS]-[napDNAbp domain]-COOH;
NH₂-[second adenosine deaminase]-[first adenosine deaminase]-[napDNAbp domain]-[NLS]—COOH;
NH₂—[NLS]-[second adenosine deaminase]-[napDNAbp domain]-[first adenosine deaminase]-COOH;
NH₂-[second adenosine deaminase]-[NLS]-[napDNAbp domain]-[first adenosine deaminase]-COOH;
NH₂-[second adenosine deaminase]-[napDNAbp domain]-[NLS]-[first adenosine deaminase]-COOH;
NH₂-[second adenosine deaminase]-[napDNAbp domain]-[first adenosine deaminase]-[NLS]—COOH;
NH₂—[NLS]-[napDNAbp domain]-[second adenosine deaminase]-[first adenosine deaminase]-COOH;
NH₂-[napDNAbp domain]-[NLS]-[second adenosine deaminase]-[first adenosine deaminase]-COOH;
NH₂-[napDNAbp domain]-[second adenosine deaminase]-[NLS]-[first adenosine deaminase]-COOH; or
NH₂-[napDNAbp domain]-[second adenosine deaminase]-[first adenosine deaminase]-[NLS]—COOH.

Exemplary base editors comprising a first adenosine deaminase, a second adenosine deaminase, a napDNAbp domain, and two NLSs may have the following architecture:
NH₂—[NLS]-[adenosine deaminase]-[napDNAbp domain]-[NLS]—COOH;
NH₂—[NLS]-[napDNAbp domain]-[adenosine deaminase]-[NLS]—COOH;
NH₂—[NLS]-[adenosine deaminase]-[napDNAbp domain]-[NLS]—COOH; or
NH₂—[NLS]-[napDNAbp domain]-[adenosine deaminase]-[NLS]—COOH.

Other exemplary base editors comprising a first adenosine deaminase, a second adenosine deaminase, a napDNAbp domain, and two NLSs may have the following architecture:
NH₂—[NLS]-[first adenosine deaminase]-[second adenosine deaminase]-[napDNAbp domain]-[NLS]—COOH;
NH₂—[NLS]-[second adenosine deaminase]-[first adenosine deaminase]-[napDNAbp domain]-[NLS]—COOH;
NH₂—[NLS]-[napDNAbp domain]-[first adenosine deaminase]-[second adenosine deaminase]-[NLS]—COOH;
NH₂—[NLS]-[napDNAbp domain]-[second adenosine deaminase]-[first adenosine deaminase]-[NLS]—COOH;
NH₂—[NLS]-[first adenosine deaminase]-[napDNAbp domain]-[second adenosine deaminase]-[NLS]—COOH;
NH₂—[NLS]-[second adenosine deaminase]-[napDNAbp domain]-[first adenosine deaminase]-[NLS]—COOH;
NH₂—[NLS]-[first adenosine deaminase]-[second adenosine deaminase]-[napDNAbp domain]-[NLS]—COOH;
NH₂—[NLS]-[second adenosine deaminase]-[first adenosine deaminase]-[napDNAbp domain]-[NLS]—COOH;
NH₂—[NLS]-[napDNAbp domain]-[first adenosine deaminase]-[second adenosine deaminase]-[NLS]—COOH;
NH₂—[NLS]-[napDNAbp domain]-[second adenosine deaminase]-[first adenosine deaminase]-[NLS]—COOH;
NH₂—[NLS]-[first adenosine deaminase]-[napDNAbp domain]-[second adenosine deaminase]-[NLS]—COOH; or
NH₂—[NLS]-[second adenosine deaminase]-[napDNAbp domain]-[first adenosine deaminase]-[NLS]—COOH.

In particular embodiments, the disclosed base editors comprise the architecture: NH₂-[bpNLS]-[TadA-8e]-[napDNAbp domain]-[bpNLS]—COOH;
NH₂-[bpNLS]-[napDNAbp domain]-[TadA-8e]-[bpNLS]—COOH;
NH₂-[bpNLS]-[TadA-8e]-[napDNAbp domain]-[bpNLS]—COOH;
NH₂-[bpNLS]-[napDNAbp domain]-[TadA-8e]-[bpNLS]—COOH;
NH₂-[bpNLS]-[wt ecTadA]-[TadA-8e]-[napDNAbp domain]-[bpNLS]—COOH;
NH₂-[bpNLS]-[TadA-8e]-[wt ecTadA]-[napDNAbp domain]-[bpNLS]—COOH;
NH₂-[bpNLS]-[napDNAbp domain]-[wt ecTadA]-[TadA-8e]-[bpNLS]—COOH;
NH₂-[bpNLS]-[napDNAbp domain]-[TadA-8e]-[wt ecTadA]-[bpNLS]—COOH;
NH₂-[bpNLS]-[wt ecTadA]-[napDNAbp domain]-[TadA-8e]-[bpNLS]—COOH;
NH₂-[bpNLS]-[TadA-8e]-[napDNAbp domain]-[wt ecTadA]-[bpNLS]—COOH;
NH₂-[bpNLS]-[wt ecTadA]-[TadA-8e]-[napDNAbp domain]-[bpNLS]—COOH;
NH₂-[bpNLS]-[TadA-8e]-[wt ecTadA]-[napDNAbp domain]-[bpNLS]—COOH;
NH₂-[bpNLS]-[napDNAbp domain]-[wt ecTadA]-[TadA-8e]-[bpNLS]—COOH;
NH₂-[bpNLS]-[napDNAbp domain]-[TadA-8e]-[wt ecTadA]-[bpNLS]—COOH;
NH₂-[bpNLS]-[wt ecTadA]-[napDNAbp domain]-[TadA-8e]-[bpNLS]—COOH; or
NH₂-[bpNLS]-[TadA-8e]-[napDNAbp domain]-[wt ecTadA]-[bpNLS]—COOH.

A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 408)); (ii) a bipartite motif consisting of two basic domains separated by a variable number of spacer amino acids and exemplified by the Xenopus nucleoplasmin NLS (KRXXXXXXXXXXKKKL (SEQ ID NO: 486)); and (iii) noncanonical sequences such as M9 of the hnRNP A1 protein, the influenza virus nucleoprotein NLS, and the yeast Gal4 protein NLS (Dingwall and Laskey, Trends Biochem Sci. 1991 December; 16(12):478-81).

Nuclear localization signals appear at various points in the amino acid sequences of proteins. NLSs have been identified at the N-terminus, the C-terminus, and in the central region of proteins. Thus, the specification provides base editors that may be modified with one or more NLSs at the C-terminus, the N-terminus, as well as at in internal region of the base editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a fusion protein (or base editor) to include one or more NLSs. In one aspect, the base editors can be engineered to express a fusion protein that is translationally fused at its N-terminus or its C-terminus (or both) to one or more NLSs, i.e., to form a fusion protein-NLS fusion construct. In other embodiments, the fusion protein-encoding nucleotide sequence can be genetically modified to incorporate a reading frame that encodes one or more NLSs in an internal region of the encoded fusion protein. In addition, the NLSs may include various amino acid linkers or spacer regions encoded between the fusion protein and the N-terminally, C-terminally, or internally-attached NLS amino acid sequence. Thus, the present disclosure also provides for nucleotide constructs, vectors, and host cells for expressing base editors that comprise a fusion protein and one or more NLSs.

The base editors described herein may also comprise nuclear localization signals which are linked to a fusion protein through one or more linkers, e.g., polymeric, amino acid, polysaccharide, chemical, or nucleic acid linker element. In certain embodiments, the NLS is linked to a fusion protein using an XTEN linker, as set forth in SEQ ID NO: 412. The linkers within the contemplated scope of the disclosure are not intended to have any limitations and can be any suitable type of molecule (e.g., polymer, amino acid, polysaccharide, nucleic acid, lipid, or any synthetic chemical linker domain) and be joined to the fusion protein by any suitable strategy that effectuates forming a bond (e.g., covalent linkage, hydrogen bonding) between the fusion protein and the one or more NLSs.

The base editors described herein also may include one or more additional elements. In certain embodiments, an additional element may comprise an effector of base repair, such as an inhibitor of base repair.

In some embodiments, the base editors described herein may comprise one or more heterologous protein domains (e.g., about or more than about 1, 2, 3, 4, 5, 6, 7, 8, 9, 10, or more domains in addition to the base editors components). A base editor may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags.

Examples of heterologous protein domains that may be fused to a base editor or component thereof (e.g., the napDNAbp domain, the nucleotide modification domain, or the NLS domain) include, without limitation, epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A base editor may be fused to a gene sequence encoding a protein or a fragment of a protein that binds DNA molecules or binds other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a base editor are described in US Patent Publication No. 2011/0059502, published Mar. 10, 2011, and incorporated herein by reference in its entirety.

In an aspect of the disclosure, a reporter gene which includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In certain embodiments of the disclosure the gene product is luciferase. In a further embodiment of the disclosure the expression of the gene product is decreased.

Other exemplary features that may be present are tags that are useful for solubilization, purification, or detection of the base editor. Suitable protein tags provided herein include, but are not limited to, biotin carboxylase carrier protein (BCCP) tags, myc-tags, calmodulin-tags, FLAG-tags, hemagglutinin (HA)-tags, bgh-PolyA tags, polyhistidine tags, and also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the base editor comprises one or more His tags.

### Linkers

In certain embodiments, linkers may be used to link any of the peptides or peptide domains or domains of the base editor (e.g., a napDNAbp domain covalently linked to an adenosine deaminase domain which is covalently linked to an NLS domain). The base editors described herein may comprise linkers of 32 amino acids in length.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35-40, 40-45, 45-50, 50-60, 60-70, 70-80, 80-90, 90-100, 100-110, 110-120, 120-130, 130-140, 140-150, or 150-200 amino acids in length. Longer or shorter linkers are also contemplated. In some embodiments, the linker is 32 amino acids in length. In exemplary embodiments, the linker comprises the 32-amino acid sequence SGGSSGGSSGSETPGTSESATPESSGGSSGGS (SEQ ID NO: 412), also known as an XTEN linker. In some embodiments, the linker comprises the 9-amino acid sequence SGGSGGSGGS (SEQ ID NO: 413). In some embodiments, the linker comprises the 4-amino acid sequence SGGS (SEQ ID NO: 414).

In some embodiments, the linker comprises the amino acid sequence (GGGGS)ₙ (SEQ ID NO: 415), (G)ₙ (SEQ ID NO: 416), (EAAAK)ₙ (SEQ ID NO: 417), (GGS)ₙ (SEQ ID NO: 418), (SGGS)ₙ (SEQ ID NO: 419), (XP)ₙ (SEQ ID NO: 420), or any combination thereof, wherein n is independently an integer between 1 and 30, and wherein X is any amino acid. In some embodiments, the linker comprises the amino acid sequence (GGS)ₙ (SEQ ID NO: 421), wherein n is 1, 3, or 7. In some embodiments, the linker comprises the amino acid sequence SGSETPGTSESATPES (SEQ ID NO: 422).

In some embodiments, a linker comprises SGSETPGTSESATPES (SEQ ID NO: 422), and SGGS (SEQ ID NO: 413). In some embodiments, a linker comprises SGGSSGSETPGTSESATPESSGGS (SEQ ID NO: 423). In some embodiments, a linker comprises SGGSSGGSSGSETPGTSESATPESSGGSSGGS (SEQ ID NO: 412). In some embodiments, a linker comprises GGSGGSPGSPAGSPTSTEEGTSESATPESGPGTSTEPSEGSAPGSPAGSPTSTEEGTSTEPSE GSAPGTSTEPSEGSAPGTSESATPESGPGSEPATSGGSGGS (SEQ ID NO: 424). In some embodiments, the linker is 24 amino acids in length. In some embodiments, the linker comprises the amino acid sequence SGGSSGGSSGSETPGTSESATPES (SEQ ID NO: 425). In some embodiments, the linker is 40 amino acids in length. In some embodiments, the linker comprises the amino acid sequence SGGSSGGSSGSETPGTSESATPESSGGSSGGSSGGSSGGS (SEQ ID NO: 426). In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGSSG GS (SEQ ID NO: 427). In some embodiments, the linker is 92 amino acids in length. In some embodiments, the linker comprises the amino acid sequence PGSPAGSPTSTEEGTSESATPESGPGTSTEPSEGSAPGSPAGSPTSTEEGTSTEPSEGSAPGT STEPSEGSAPGTSESATPESGPGSEPATS (SEQ ID NO: 428). It should be appreciated that any of the linkers provided herein may be used to link a first adenosine deaminase and a second adenosine deaminase; an adenosine deaminase domain (comprising, e.g., a first and/or a second adenosine deaminase) and a napDNAbp; a napDNAbp and an NLS; or an adenosine deaminase domain and an NLS.

In some embodiments, any of the base editors provided herein, comprise an adenosine deaminase and a napDNAbp that are fused to each other via a linker. In some embodiments, any of the base editors provided herein, comprise a first adenosine deaminase and a second adenosine deaminase that are fused to each other via a linker. In some embodiments, any of the base editors provided herein, comprise an NLS, which may be fused to an adenosine deaminase (e.g., a first and/or a second adenosine deaminase) and a nucleic acid programmable DNA binding protein (napDNAbp). Various linker lengths and flexibilities between an adenosine deaminase (e.g., an engineered ecTadA) and a napDNAbp (e.g., a Cas9 domain), and/or between a first adenosine deaminase and a second adenosine deaminase may be employed (e.g., ranging from very flexible linkers of the form of SEQ ID NOs: 119, 121-124 (see, e.g., Guilinger J P, Thompson D B, Liu D R. Fusion of catalytically inactive Cas9 to Fokl nuclease improves the specificity of genome modification. Nat. Biotechnol. 2014; 32(6): 577-82; the entire contents are incorporated herein by reference) and (XP)ₙ (SEQ ID NO: 420)) in order to achieve the optimal length for deaminase activity for the specific application. In some embodiments, n is 1, 2, 3, 4, 5, 6, 7, 8, 9, 10, 11, 12, 13, 14, or 15. In some embodiments, the linker comprises a (GGS)ₙ (SEQ ID NO: 421) motif, wherein n is 1, 3, or 7. In some embodiments, the adenosine deaminase and the napDNAbp, and/or the first adenosine deaminase and the second adenosine deaminase of any of the base editors provided herein are fused via a linker comprising an amino acid sequence selected from SEQ ID NOs: 119-132. In some embodiments, the linker is 24 amino acids in length. In some embodiments, the linker comprises the amino acid sequence (SGGS)₂-SGSETPGTSESATPES-(SGGS)₂ (SEQ ID NO: 412), which may also be referred to as (SGGS)₂-XTEN-(SGGS)₂ (SEQ ID NO: 429). In some embodiments, the linker comprises the amino acid sequence, wherein n is 0, 1, 2, 3, 4, 5, 6, 7, 8, 9, or 10. In some embodiments, the linker is 40 amino acids in length. In some embodiments, the linker is 64 amino acids in length. In some embodiments, the linker is 92 amino acids in length.

### Exemplary Adenine Base Editors

Some aspects of the disclosure provide base editors comprising an adenine base editor comprising a napDNAbp domain (e.g. an nCas9 domain) and an adenosine deaminase domain (e.g., a heterodimer of adenosine deaminases). In some embodiments, the base editors comprise adenine base editors for multiplexing applications. In still other embodiments, the base editors comprise ancestrally reconstructed adenine base editors.

The present disclosure provides newly discovered mutations in TadA 7.10 (SEQ ID NO: 315) (the TadA* used in ABEmax) that yield adenosine deaminase variants and confer broader Cas compatibility to the deaminase (see FIG. 2A). In certain embodiments, these mutations confer reduced off-target effects, such as reduced RNA editing activity and off-target DNA editing activity, on the adenine base editor. The adenine base editors of the present disclosure comprise one or more of the disclosed adenosine deaminase variants. In other embodiments, the adenine base editors may comprise one or more adenosine deaminases having two or more such substitutions in combination. In some embodiments, the adenine base editors comprise adenosine deaminases comprising comprises a sequence with at least 80%, 85%, 90%, 95%, 98%, 99%, or 99.5% sequence identity to SEQ ID NO: 433 (TadA-8e).

In some embodiments, the adenine base editor of the disclosure comprises an amino acid sequence that is at least 80%, 85%, 90%, 95%, 98%, 99%, or 99.5% identical to the amino acid sequence of any one of SEQ ID NOs: 169-188, 449-462, and 465-476. In particular embodiments, the adenine base editor of the disclosure comprises any one of the sequences set forth as SEQ ID NOs: 169-188, 449-462, and 465-476.

In some embodiments, the adenine base editor of the disclosure comprises the sequence of SEQ ID NO: 138. In some embodiments, the adenine base editor of the disclosure comprises the sequence of SEQ ID NO: 142. In other embodiments, the adenine base editor of the disclosure comprises a sequence selected from SEQ ID NOs: 143-146. In other embodiments, the adenine base editor of the disclosure comprises the sequence of SEQ ID NO: 147. In other embodiments, the adenine base editor of the disclosure comprises the sequence of SEQ ID NOs: 148 or 149. In other embodiments, the adenine base editors of the disclosure comprises the sequence of SEQ ID NOs: 171 or 172. In other embodiments, the adenine base editor of the disclosure comprises the sequence of SEQ ID NOs: 183 or 185. In other embodiments, the adenine base editor of the disclosure comprises the sequence of SEQ ID NO: 469 or SEQ ID NO: 453.

In some embodiments, any of the adenine base editors described herein may comprise an amino acid sequence having 1, 2, 3, 4, 5, 6, 7, 8, 9, 10, 11, 12, 13, 14, 15, 16, 17, 18, 19, 20, 25, 30, or more than 30 amino acids that differ relative to the amino acid sequence of any of SEQ ID NOs: 169-188, 449-462, and 465-476. These differences may comprise amino acids that have been inserted, deleted, or substituted relative to the reference sequence. In some embodiments, the disclosed adenosine deaminase domains contain stretches of about 50, about 75, about 100, about 125, about 150, about 175, about 200, about 300, about 400, about 500, or more than 500 consecutive amino acids in common with either of SEQ ID NOs: 169-188, 449-462, and 465-476.

Exemplary adenine base editors of this disclosure comprise the monomer and dimer versions of the following editors: ABE8e, SaABE8e, SaKKH-ABE8e, NG-ABE8e, ABE-xCas9, ABE8e-NRTH, ABE8e-NRRH, ABE8e-NRCH, ABE8e-NG-CP1041, ABE8e-VRQR-CP1041, ABE8e-CP1041, ABE8e-CP1028, ABE8e-VRQR, ABE8e-LbCas12a (LbABE8e), ABE8e-AsCas12a (enAsABE8e), ABE8e-SpyMac, ABE8e (TadA-8e V106W), ABE8e (K20A,R21A), and ABE8e(TadA-8e V82G). The monomer version refers to an editor having an adenosine deaminase domain that comprises a TadA8e and does not comprise a second adenosine deaminase enzyme. The dimer version refers to an editor having an adenosine deaminase domain that comprises a first and second adenosine deaminase, i.e., a wild-type TadA enzyme and a TadA8e enzyme.

Exemplary base editors comprise sequences that are at least 85%, at least 90%, at least 95%, at least 98%, at least 99%, or at least 99.5% identical to any of the following amino acid sequences:

ABEmax, or ABE7.10
(SEQ ID NO: 487)
MSEVEFSHEYWMRHALTLAKRAWDEREVPVGAVLVHNNRVIGEGWNRPIGRHDPTAHAEIMAL
RQGGLVMQNYRLIDATLYVTLEPCVMCAGAMIHSRIGRVVFGARDAKTGAAGSLMDVLHHPGM
NHRVEITEGILADECAALLSDFFRMRRQEIKAQKKAQSSTDSGGSSGGSSGSETPGTSESATPESSG
GSSGGSSEVEFSHEYWMRHALTLAKRARDEREVPVGAVLVLNNRVIGEGWNRAIGLHDPTAHAE
IMALRQGGLVMQNYRLIDATLYVTFEPCVMCAGAMIHSRIGRVVFGVRNAKTGAAGSLMDVLH
YPGMNHRVEITEGILADECAALLCYFFRMPRQVFNAQKKAQSSTDSGGSSGGSSGSETPGTSESA
TPESSGGSSGGSDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKLFIQ
LVQTYNQLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKVLPKHSLLYEY
FTVYNELTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQKGQKNS
RERMKRIEEGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAKY
FFYSNIMNFFKTEITLANGEIRKRPLIETNGETGEIVWDKGRDFATVRKVLSMPQVNIVKKTEVQT
GGFSKESILPKRNSDKLIARKKDWDPKKYGGFDSPTVAYSVLVVAKVEKGKSKKLKSVKELLGIT
IMERSSFEKNPIDFLEAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRIDLS
QLGGDSGGSPKKKRKV
ABE8e
(SEQ ID NO: 169)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRNS
KRGAAGSLMNVLNYPGMNHRVEITEGILADECAALLCDFYRMPRQVFNAQKKAQSSINSGGSSG
GSSGSETPGTSESATPESSGGSSGGSDKKYSIGLAIGTNSVGWAVITDEYKVPSKKFKVLGNTDRH
SIKKNLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDY
FKKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMYVDQELD
INRLSDYDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDSGGSKRTADGSEFEPKKKRKV
ABE8e-dimer
(SEQ ID NO: 170)
MKRTADGSEFESPKKKRKVSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTDSGGSSG
GSSGSETPGTSESATPESSGGSSGGSSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGRVVFG
VRNSKRGAAGSLMNVLNYPGMNHRVEITEGILADECAALLCDFYRMPRQVFNAQKKAQSSINS
GGSSGGSSGSETPGTSESATPESSGGSSGGSDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRLIYLALAHMIKFRGHF
LIEGDLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNFM
QLIHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDMY
VDQELDINRLSDYDVDHIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEFVYGDYKVYD
VRKMIAKSEQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYTSTKEVLDAT
LIHQSITGLYETRIDLSQLGGDSGGSKRTADGSEFEPKKKRKV
SaABE8e
(SEQ ID NO: 171)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRNS
KRGAAGSLMNVLNYPGMNHRVEITEGILADECAALLCDFYRMPRQVFNAQKKAQSSINSGGSSG
GSSGSETPGTSESATPESSGGSSGGSGKRNYILGLAIGITSVGYGIIDYETRDVIDAGVRLFKEANV
ENNEGRRSKRGARRLKRRRRHRIQRVKKLLFDYNLLTDHSELSGINPYEARVKGLSQKLSEEEFS
AALLHLAKRRGVHNVNEVEEDTGNELSTKEQISRNSKALEEKYVAELQLERLKKDGEVRGSINR
FKTSDYVKEAKQLLKVQKAYHQLDQSFIDTYIDLLETRRTYYEGPGEGSPFGWKDIKEWYEMLM
GHCTYFPEELRSVKYAYNADLYNALNDLNNLVITRDENEKLEYYEKFQIIENVFKQKKKPTLKQI
AKEILVNEEDIKGYRVTSTGKPEFTNLKVYHDIKDITARKEIIENAELLDQIAKILTIYQSSEDIQEEL
TNLNSELTQEEIEQISNLKGYTGTHNLSLKAINLILDELWHTNDNQIAIFNRLKLVPKKVDLSQQKE
IPTTLVDDFILSPVVKRSFIQSIKVINAIIKKYGLPNDIIIELAREKNSKDAQKMINEMQKRNRQTNE
RIEEIIRTTGKENAKYLIEKIKLHDMQEGKCLYSLEAIPLEDLLNNPFNYEVDHIIPRSVSFDNSFNN
KVLVKQEENSKKGNRTPFQYLSSSDSKISYETFKKHILNLAKGKGRISKTKKEYLLEERDINRFSV
QKDFINRNLVDTRYATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYKEIFITPHQIKHIKDFK
DYKYSHRVDKKPNRELINDTLYSTRKDDKGNTLIVNNLNGLYDKDNDKLKKLINKSPEKLLMYH
HDPQTYQKLKLIMEQYGDEKNPLYKYYEETGNYLTKYSKKDNGPVIKKIKYYGNKLNAHLDITD
DYPNSRNKVVKLSLKPYRFDVYLDNGVYKFVTVKNLDVIKKENYYEVNSKCYEEAKKLKKISN
QAEFIASFYNNDLIKINGELYRVIGVNNDLLNRIEVNMIDITYREYLENMNDKRPPRIIKTIASKTQS
IKKYSTDILGNLYEVKSKKHPQIIKKGSGGSKRTADGSEFEPKKKRKV
SaABE8e-dimer
(SEQ ID NO: 172)
MKRTADGSEFESPKKKRKVSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTDSGGSSG
GSSGSETPGTSESATPESSGGSSGGSSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGRVVFG
VRNSKRGAAGSLMNVLNYPGMNHRVEITEGILADECAALLCDFYRMPRQVFNAQKKAQSSINS
GGSSGGSSGSETPGTSESATPESSGGSSGGSGKRNYILGLAIGITSVGYGIIDYETRDVIDAGVRLFK
EANVENNEGRRSKRGARRLKRRRRHRIQRVKKLLFDYNLLTDHSELSGINPYEARVKGLSQKLSE
EEFSAALLHLAKRRGVHNVNEVEEDTGNELSTKEQISRNSKALEEKYVAELQLERLKKDGEVRG
SINRFKTSDYVKEAKQLLKVQKAYHQLDQSFIDTYIDLLETRRTYYEGPGEGSPFGWKDIKEWYE
MLMGHCTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIYQSSEDI
QEELTNLNSELTQEEIEQISNLKGYTGTHNLSLKAINLILDELWHTNDNQIAIFNRLKLVPKKVDLS
QQKEIPTTLVDDFILSPVVKRSFIQSIKVINAIIKKYGLPNDIIIELAREKNSKDAQKMINEMQKRNR
QTNERIEEIIRTTGKENAKYLIEKIKLHDMQEGKCLYSLEAIPLEDLLNNPFNYEVDHIIPRSVSFDN
SFNNKVLVKQEENSKKGNRTPFQYLSSSDSKISYETFKKHILNLAKGKGRISKTKKEYLLEERDIN
RFSVQKDFINRNLVDTRYATRGLMNLLRSYFRVNNLDVKVKSINGGFTSFLRRKWKFKKERNKG
YKHHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSPEKL
LMYHHDPQTYQKLKLIMEQYGDEKNPLYKYYEETGNYLTKYSKKDNGPVIKKIKYYGNKLNAH
LDITDDYPNSRNKVVKLSLKPYRFDVYLDNGVYKFVTVKNLDVIKKENYYEVNSKCYEEAKKL
KKISNQAEFIASFYNNDLIKINGELYRVIGVNNDLLNRIEVNMIDITYREYLENMNDKRPPRIIKTIA
SKTQSIKKYSTDILGNLYEVKSKKHPQIIKKGSGGSKRTADGSEFEPKKKRKV
LbABE8e
(SEQ ID NO: 173)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRNS
KRGAAGSLMNVLNYPGMNHRVEITEGILADECAALLCDFYRMPRQVFNAQKKAQSSINSGGSSG
GSSGSETPGTSESATPESSGGSSGGSSKLEKFTNCYSLSKTLRFKAIPVGKTQENIDNKRLLVEDEK
RAEDYKGVKKLLDRYYLSFINDVLHSIKLKNLNNYISLFRKKTRTEKENKELENLEINLRKEIAKA
FKGNEGYKSLFKKDIIETILPEFLDDKDEIALVNSFNGFTTAFTGFFDNRENMFSEEAKSTSIAFRCI
NENLTRYISNMDIFEKVDAIFDKHEVQEIKEKILNSDYDVEDFFEGEFFNFVLTQEGIDVYNAIIGG
FVTESGEKIKGLNEYINLYNQKTKQKLPKFKPLYKQVLSDRESLSFYGEGYTSDEEVLEVFRNTLN
KNSEIFSSIKKLEKLFKNFDEYSSAGIFVKNGPAISTISKDIFGEWNVIRDKWNAEYDDIHLKKKAV
VTEKYEDDRRKSFKKIGSFSLEQLQEYADADLSVVEKLKEIIIQKVDEIYKVYGSSEKLFDADFVL
EKSLKKNDAVVAIMKDLLDSVKSFENYIKAFFGEGKETNRDESFYGDFVLAYDILLKVDHIYDAI
RNYVTQKPYSKDKFKLYFQNPQFMGGWDKDKETDYRATILRYGSKYYLAIMDKKYAKCLQKID
KDDVNGNYEKINYKLLPGPNKMLPKVFFSKKWMAYYNPSEDIQKIYKNGTFKKGDMFNLNDCH
KLIDFFKDSISRYPKWSNAYDFNFSETEKYKDIAGFYREVEEQGYKVSFESASKKEVDKLVEEGK
LYMFQIYNKDFSDKSHGTPNLHTMYFKLLFDENNHGQIRLSGGAELFMRRASLKKEELVVHPAN
SPIANKNPDNPKKTTTLSYDVYKDKRFSEDQYELHIPIAINKCPKNIFKINTEVRVLLKHDDNPYVI
GIARGERNLLYIVVVDGKGNIVEQYSLNEIINNFNGIRIKTDYHSLLDKKEKERFEARQNWTSIENI
KELKAGYISQVVHKICELVEKYDAVIALEDLNSGFKNSRVKVEKQVYQKFEKMLIDKLNYMVDK
KSNPCATGGALKGYQITNKFESFKSMSTQNGFIFYIPAWLTSKIDPSTGFVNLLKTKYTSIADSKKF
ISSFDRIMYVPEEDLFEFALDYKNFSRTDADYIKKWKLYSYGNRIRIFRNPKKNNVFDWEEVCLTS
AYKELFNKYGINYQQGDIRALLCEQSDKAFYSSFMALMSLMLQMRNSITGRTDVDFLISPVKNSD
GIFYDSRNYEAQENAILPKNADANGAYNIARKVLWAIGQFKKAEDEKLDKVKIAISNKEWLEYA
QTSVKSGGSKRTADGSEFEPKKKRKV
LbABE8e-dimer
(SEQ ID NO: 174)
MKRTADGSEFESPKKKRKVSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTDSGGSSG
GSSGSETPGTSESATPESSGGSSGGSSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGRVVFG
VRNSKRGAAGSLMNVLNYPGMNHRVEITEGILADECAALLCDFYRMPRQVFNAQKKAQSSINS
GGSSGGSSGSETPGTSESATPESSGGSSGGSSKLEKFTNCYSLSKTLRFKAIPVGKTQENIDNKRLL
VEDEKRAEDYKGVKKLLDRYYLSFINDVLHSIKLKNLNNYISLFRKKTRTEKENKELENLEINLR
KEIAKAFKGNEGYKSLFKKDIIETILPEFLDDKDEIALVNSFNGFTTAFTGFFDNRENMFSEEAKST
SIAFRCINENLTRYISNMDIFEKVDAIFDKHEVQEIKEKILNSDYDVEDFFEGEFFNFVLTQEGIDVY
NAIIGGFVTESGEKIKGLNEYINLYNQKTKQKLPKFKPLYKQVLSDRESLSFYGEGYTSDEEVLEV
FRNTLNKNSEIFSSIKKLEKLFKNFDEYSSAGIFVKNGPAISTISKDIFGEWNVIRDKWNAEYDDIH
LKKKAVVTEKYEDDRRKSFKKIGSFSLEQLQEYADADLSVVEKLKEIIIQKVDEIYKVYGSSEKLF
DADFVLEKSLKKNDAVVAIMKDLLDSVKSFENYIKAFFGEGKETNRDESFYGDFVLAYDILLKVD
HIYDAIRNYVTQKPYSKDKFKLYFQNPQFMGGWDKDKETDYRATILRYGSKYYLAIMDKKYAK
CLQKIDKDDVNGNYEKINYKLLPGPNKMLPKVFFSKKWMAYYNPSEDIQKIYKNGTFKKGDMF
NLNDCHKLIDFFKDSISRYPKWSNAYDFNFSETEKYKDIAGFYREVEEQGYKVSFESASKKEVDK
LVEEGKLYMFQIYNKDFSDKSHGTPNLHTMYFKLLFDENNHGQIRLSGGAELFMRRASLKKEEL
VVHPANSPIANKNPDNPKKTTTLSYDVYKDKRFSEDQYELHIPIAINKCPKNIFKINTEVRVLLKH
DDNPYVIGIARGERNLLYIVVVDGKGNIVEQYSLNEIINNFNGIRIKTDYHSLLDKKEKERFEARQ
NWTSIENIKELKAGYISQVVHKICELVEKYDAVIALEDLNSGFKNSRVKVEKQVYQKFEKMLIDK
LNYMVDKKSNPCATGGALKGYQITNKFESFKSMSTQNGFIFYIPAWLTSKIDPSTGFVNLLKTKYT
SIADSKKFISSFDRIMYVPEEDLFEFALDYKNFSRTDADYIKKWKLYSYGNRIRIFRNPKKNNVFD
WEEVCLTSAYKELFNKYGINYQQGDIRALLCEQSDKAFYSSFMALMSLMLQMRNSITGRTDVDF
LISPVKNSDGIFYDSRNYEAQENAILPKNADANGAYNIARKVLWAIGQFKKAEDEKLDKVKIAIS
NKEWLEYAQTSVKSGGSKRTADGSEFEPKKKRKV
LbABE7.10
(SEQ ID NO: 175)
MKRTADGSEFESPKKKRKVSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTDSGGSSG
GSSGSETPGTSESATPESSGGSSGGSSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGRVVFG
VRNAKTGAAGSLMDVLHYPGMNHRVEITEGILADECAALLCYFFRMPRQVFNAQKKAQSSTDS
GGSSGGSSGSETPGTSESATPESSGGSSGGSSKLEKFTNCYSLSKTLRFKAIPVGKTQENIDNKRLL
VEDEKRAEDYKGVKKLLDRYYLSFINDVLHSIKLKNLNNYISLFRKKTRTEKENKELENLEINLR
KEIAKAFKGNEGYKSLFKKDIIETILPEFLDDKDEIALVNSFNGFTTAFTGFFDNRENMFSEEAKST
SIAFRCINENLTRYISNMDIFEKVDAIFDKHEVQEIKEKILNSDYDVEDFFEGEFFNFVLTQEGIDVY
NAIIGGFVTESGEKIKGLNEYINLYNQKTKQKLPKFKPLYKQVLSDRESLSFYGEGYTSDEEVLEV
FRNTLNKNSEIFSSIKKLEKLFKNFDEYSSAGIFVKNGPAISTISKDIFGEWNVIRDKWNAEYDDIH
LKKKAVVTEKYEDDRRKSFKKIGSFSLEQLQEYADADLSVVEKLKEIIIQKVDEIYKVYGSSEKLF
DADFVLEKSLKKNDAVVAIMKDLLDSVKSFENYIKAFFGEGKETNRDESFYGDFVLAYDILLKVD
HIYDAIRNYVTQKPYSKDKFKLYFQNPQFMGGWDKDKETDYRATILRYGSKYYLAIMDKKYAK
CLQKIDKDDVNGNYEKINYKLLPGPNKMLPKVFFSKKWMAYYNPSEDIQKIYKNGTFKKGDMF
NLNDCHKLIDFFKDSISRYPKWSNAYDFNFSETEKYKDIAGFYREVEEQGYKVSFESASKKEVDK
LVEEGKLYMFQIYNKDFSDKSHGTPNLHTMYFKLLFDENNHGQIRLSGGAELFMRRASLKKEEL
VVHPANSPIANKNPDNPKKTTTLSYDVYKDKRFSEDQYELHIPIAINKCPKNIFKINTEVRVLLKH
DDNPYVIGIARGERNLLYIVVVDGKGNIVEQYSLNEIINNFNGIRIKTDYHSLLDKKEKERFEARQ
NWTSIENIKELKAGYISQVVHKICELVEKYDAVIALEDLNSGFKNSRVKVEKQVYQKFEKMLIDK
LNYMVDKKSNPCATGGALKGYQITNKFESFKSMSTQNGFIFYIPAWLTSKIDPSTGFVNLLKTKYT
SIADSKKFISSFDRIMYVPEEDLFEFALDYKNFSRTDADYIKKWKLYSYGNRIRIFRNPKKNNVFD
WEEVCLTSAYKELFNKYGINYQQGDIRALLCEQSDKAFYSSFMALMSLMLQMRNSITGRTDVDF
LISPVKNSDGIFYDSRNYEAQENAILPKNADANGAYNIARKVLWAIGQFKKAEDEKLDKVKIAIS
NKEWLEYAQTSVKSGGSKRTADGSEFEPKKKRKV
enAsABE8e
(SEQ ID NO: 176)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRNS
KRGAAGSLMNVLNYPGMNHRVEITEGILADECAALLCDFYRMPRQVFNAQKKAQSSINSGGSSG
GSSGSETPGTSESATPESSGGSSGGSMTQFEGFTNLYQVSKTLRFELIPQGKTLKHIQEQGFIEEDK
ARNDHYKELKPIIDRIYKTYADQCLQLVQLDWENLSAAIDSYRKEKTEETRNALIEEQATYRNAIH
DYFIGRTDNLTDAINKRHAEIYKGLFKAELFNGKVLKQLGTVTTTEHENALLRSFDKFTTYFSGF
YRNRKNVFSAEDISTAIPHRIVQDNFPKFKENCHIFTRLITAVPSLREHFENVKKAIGIFVSTSIEEVF
SFPFYNQLLTQTQIDLYNQLLGGISREAGTEKIKGLNEVLNLAIQKNDETAHIIASLPHRFIPLFKQI
LSDRNTLSFILEEFKSDEEVIQSFCKYKTLLRNENVLETAEALFNELNSIDLTHIFISHKKLETISSAL
CDHWDTLRNALYERRISELTGKITKSAKEKVQRSLKHEDINLQEIISAAGKELSEAFKQKTSEILSH
AHAALDQPLPTTLKKQEEKEILKSQLDSLLGLYHLLDWFAVDESNEVDPEFSARLTGIKLEMEPSL
SFYNKARNYATKKPYSVEKFKLNFQMPTLARGWDVNREKNNGAILFVKNGLYYLGIMPKQKGR
YKALSFEPTEKTSEGFDKMYYDYFPDAAKMIPKCSTQLKAVTAHFQTHTTPILLSNNFIEPLEITKE
IYDLNNPEKEPKKFQTAYAKKTGDQKGYREALCKWIDFTRDFLSKYTKTTSIDLSSLRPSSQYKD
LGEYYAELNPLLYHISFQRIAEKEIMDAVETGKLYLFQIYNKDFAKGHHGKPNLHTLYWTGLFSPE
NLAKTSIKLNGQAELFYRPKSRMKRMAHRLGEKMLNKKLKDQKTPIPDTLYQELYDYVNHRLS
HDLSDEARALLPNVITKEVSHEIIKDRRFTSDKFFFHVPITLNYQAANSPSKFNQRVNAYLKEHPE
TPIIGIARGERNLIYITVIDSTGKILEQRSLNTIQQFDYQKKLDNREKERVAARQAWSVVGTIKDLK
QGYLSQVIHEIVDLMIHYQAVVVLENLNFGFKSKRTGIAEKAVYQQFEKMLIDKLNCLVLKDYPA
EKVGGVLNPYQLTDQFTSFAKMGTQSGFLFYVPAPYTSKIDPLTGFVDPFVWKTIKNHESRKHFL
EGFDFLHYDVKTGDFILHFKMNRNLSFQRGLPGFMPAWDIVFEKNETQFDAKGTPFIAGKRIVPVI
ENHRFTGRYRDLYPANELIALLEEKGIVFRDGSNILPKLLENDDSHAIDTMVALIRSVLQMRNSNA
ATGEDYINSPVRDLNGVCFDSRFQNPEWPMDADANGAYHIALKGQLLLNHLKESKDLKLQNGIS
NQDWLAYIQELRNSGGSKRTADGSEFEPKKKRKV
enAsABE8e-dimer
(SEQ ID NO: 177)
MKRTADGSEFESPKKKRKVSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTDSGGSSG
GSSGSETPGTSESATPESSGGSSGGSSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGRVVFG
VRNSKRGAAGSLMNVLNYPGMNHRVEITEGILADECAALLCDFYRMPRQVFNAQKKAQSSINS
GGSSGGSSGSETPGTSESATPESSGGSSGGSMTQFEGFTNLYQVSKTLRFELIPQGKTLKHIQEQGF
IEEDKARNDHYKELKPIIDRIYKTYADQCLQLVQLDWENLSAAIDSYRKEKTEETRNALIEEQATY
RNAIHDYFIGRTDNLTDAINKRHAEIYKGLFKAELFNGKVLKQLGTVTTTEHENALLRSFDKFTT
YFSGFYRNRKNVFSAEDISTAIPHRIVQDNFPKFKENCHIFTRLITAVPSLREHFENVKKAIGIFVST
SIEEVFSFPFYNQLLTQTQIDLYNQLLGGISREAGTEKIKGLNEVLNLAIQKNDETAHIIASLPHRFIP
LFKQILSDRNTLSFILEEFKSDEEVIQSFCKYKTLLRNENVLETAEALFNELNSIDLTHIFISHKKLET
ISSALCDHWDTLRNALYERRISELTGKITKSAKEKVQRSLKHEDINLQEIISAAGKELSEAFKQKTS
EILSHAHAALDQPLPTTLKKQEEKEILKSQLDSLLGLYHLLDWFAVDESNEVDPEFSARLTGIKLE
MEPSLSFYNKARNYATKKPYSVEKFKLNFQMPTLARGWDVNREKNNGAILFVKNGLYYLGIMP
KQKGRYKALSFEPTEKTSEGFDKMYYDYFPDAAKMIPKCSTQLKAVTAHFQTHTTPILLSNNFIEP
LEITKEIYDLNNPEKEPKKFQTAYAKKTGDQKGYREALCKWIDFTRDFLSKYTKTTSIDLSSLRPS
SQYKDLGEYYAELNPLLYHISFQRIAEKEIMDAVETGKLYLFQIYNKDFAKGHHGKPNLHTLYWT
GLFSPENLAKTSIKLNGQAELFYRPKSRMKRMAHRLGEKMLNKKLKDQKTPIPDTLYQELYDYV
NHRLSHDLSDEARALLPNVITKEVSHEIIKDRRFTSDKFFFHVPITLNYQAANSPSKFNQRVNAYL
KEHPETPIIGIARGERNLIYITVIDSTGKILEQRSLNTIQQFDYQKKLDNREKERVAARQAWSVVGTI
KDLKQGYLSQVIHEIVDLMIHYQAVVVLENLNFGFKSKRTGIAEKAVYQQFEKMLIDKLNCLVLK
DYPAEKVGGVLNPYQLTDQFTSFAKMGTQSGFLFYVPAPYTSKIDPLTGFVDPFVWKTIKNHESR
KHFLEGFDFLHYDVKTGDFILHFKMNRNLSFQRGLPGFMPAWDIVFEKNETQFDAKGTPFIAGKR
IVPVIENHRFTGRYRDLYPANELIALLEEKGIVFRDGSNILPKLLENDDSHAIDTMVALIRSVLQMR
NSNAATGEDYINSPVRDLNGVCFDSRFQNPEWPMDADANGAYHIALKGQLLLNHLKESKDLKL
QNGISNQDWLAYIQELRNSGGSKRTADGSEFEPKKKRKV
enAsABE7.10
(SEQ ID NO: 178)
MKRTADGSEFESPKKKRKVSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTDSGGSSG
GSSGSETPGTSESATPESSGGSSGGSSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGRVVFG
VRNAKTGAAGSLMDVLHYPGMNHRVEITEGILADECAALLCYFFRMPRQVFNAQKKAQSSTDS
GGSSGGSSGSETPGTSESATPESSGGSSGGSMTQFEGFTNLYQVSKTLRFELIPQGKTLKHIQEQGF
IEEDKARNDHYKELKPIIDRIYKTYADQCLQLVQLDWENLSAAIDSYRKEKTEETRNALIEEQATY
RNAIHDYFIGRTDNLTDAINKRHAEIYKGLFKAELFNGKVLKQLGTVTTTEHENALLRSFDKFTT
YFSGFYRNRKNVFSAEDISTAIPHRIVQDNFPKFKENCHIFTRLITAVPSLREHFENVKKAIGIFVST
SIEEVFSFPFYNQLLTQTQIDLYNQLLGGISREAGTEKIKGLNEVLNLAIQKNDETAHIIASLPHRFIP
LFKQILSDRNTLSFILEEFKSDEEVIQSFCKYKTLLRNENVLETAEALFNELNSIDLTHIFISHKKLET
ISSALCDHWDTLRNALYERRISELTGKITKSAKEKVQRSLKHEDINLQEIISAAGKELSEAFKQKTS
EILSHAHAALDQPLPTTLKKQEEKEILKSQLDSLLGLYHLLDWFAVDESNEVDPEFSARLTGIKLE
MEPSLSFYNKARNYATKKPYSVEKFKLNFQMPTLARGWDVNREKNNGAILFVKNGLYYLGIMP
KQKGRYKALSFEPTEKTSEGFDKMYYDYFPDAAKMIPKCSTQLKAVTAHFQTHTTPILLSNNFIEP
LEITKEIYDLNNPEKEPKKFQTAYAKKTGDQKGYREALCKWIDFTRDFLSKYTKTTSIDLSSLRPS
SQYKDLGEYYAELNPLLYHISFQRIAEKEIMDAVETGKLYLFQIYNKDFAKGHHGKPNLHTLYWT
GLFSPENLAKTSIKLNGQAELFYRPKSRMKRMAHRLGEKMLNKKLKDQKTPIPDTLYQELYDYV
NHRLSHDLSDEARALLPNVITKEVSHEIIKDRRFTSDKFFFHVPITLNYQAANSPSKFNQRVNAYL
KEHPETPIIGIARGERNLIYITVIDSTGKILEQRSLNTIQQFDYQKKLDNREKERVAARQAWSVVGTI
KDLKQGYLSQVIHEIVDLMIHYQAVVVLENLNFGFKSKRTGIAEKAVYQQFEKMLIDKLNCLVLK
DYPAEKVGGVLNPYQLTDQFTSFAKMGTQSGFLFYVPAPYTSKIDPLTGFVDPFVWKTIKNHESR
KHFLEGFDFLHYDVKTGDFILHFKMNRNLSFQRGLPGFMPAWDIVFEKNETQFDAKGTPFIAGKR
IVPVIENHRFTGRYRDLYPANELIALLEEKGIVFRDGSNILPKLLENDDSHAIDTMVALIRSVLQMR
NSNAATGEDYINSPVRDLNGVCFDSRFQNPEWPMDADANGAYHIALKGQLLLNHLKESKDLKL
QNGISNQDWLAYIQELRNSGGSKRTADGSEFEPKKKRKV
SpCas9NG-ABE8e (“NG-ABE8e”)
(SEQ ID NO: 179)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRNS
KRGAAGSLMNVLNYPGMNHRVEITEGILADECAALLCDFYRMPRQVFNAQKKAQSSINSGGSSG
GSSGSETPGTSESATPESSGGSSGGSDKKYSIGLAIGTNSVGWAVITDEYKVPSKKFKVLGNTDRH
SIKKNLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDY
FKKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMYVDQELD
INRLSDYDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESIRPKRNSDKLIARKKDWDPKKYGGFVSPTVAYSVLVVAKVEKG
KSKKLKSVKELLGITIMERSSFEKNPIDFLEAKGYKEVKKDLIIKLPKYSLFELENGRKRMLASAR
FLQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPRAFKYFDTTIDRKVYRSTKEVLDATLIHQ
SITGLYETRIDLSQLGGDSGGSKRTADGSEFEPKKKRKV
NG-ABE8e-dimer
(SEQ ID NO: 180)
MKRTADGSEFESPKKKRKVSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTDSGGSSG
GSSGSETPGTSESATPESSGGSSGGSSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGRVVFG
VRNSKRGAAGSLMNVLNYPGMNHRVEITEGILADECAALLCDFYRMPRQVFNAQKKAQSSINS
GGSSGGSSGSETPGTSESATPESSGGSSGGSDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRLIYLALAHMIKFRGHF
LIEGDLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLTLFEDR
EMIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKPEN
IVIEMARENQTTQKGQKNSRERMKRIEEGIKELGSQILKEHPVENTQLQNEKLYLYYLQNGRDMY
VDQELDINRLSDYDVDHIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETNGETGEIVWDKGRDFAT
VRKVLSMPQVNIVKKTEVQTGGFSKESIRPKRNSDKLIARKKDWDPKKYGGFVSPTVAYSVLVV
AKVEKGKSKKLKSVKELLGITIMERSSFEKNPIDFLEAKGYKEVKKDLIIKLPKYSLFELENGRKR
MLASARFLQKGNELALPSKYVNFLYLASHYEKLKGSPEDNEQKQLFVEQHKHYLDEIIEQISEFS
KRVILADANLDKVLSAYNKHRDKPIREQAENIIHLFTLTNLGAPRAFKYFDTTIDRKVYRSTKEVL
DATLIHQSITGLYETRIDLSQLGGDSGGSKRTADGSEFEPKKKRKV
SaKKH-ABE8e (“KKH-ABE8e”)
(SEQ ID NO: 181)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRNS
KRGAAGSLMNVLNYPGMNHRVEITEGILADECAALLCDFYRMPRQVFNAQKKAQSSINSGGSSG
GSSGSETPGTSESATPESSGGSSGGSGKRNYILGLAIGITSVGYGIIDYETRDVIDAGVRLFKEANV
ENNEGRRSKRGARRLKRRRRHRIQRVKKLLFDYNLLTDHSELSGINPYEARVKGLSQKLSEEEFS
AALLHLAKRRGVHNVNEVEEDTGNELSTKEQISRNSKALEEKYVAELQLERLKKDGEVRGSINR
FKTSDYVKEAKQLLKVQKAYHQLDQSFIDTYIDLLETRRTYYEGPGEGSPFGWKDIKEWYEMLM
GHCTYFPEELRSVKYAYNADLYNALNDLNNLVITRDENEKLEYYEKFQIIENVFKQKKKPTLKQI
AKEILVNEEDIKGYRVTSTGKPEFTNLKVYHDIKDITARKEIIENAELLDQIAKILTIYQSSEDIQEEL
TNLNSELTQEEIEQISNLKGYTGTHNLSLKAINLILDELWHTNDNQIAIFNRLKLVPKKVDLSQQKE
IPTTLVDDFILSPVVKRSFIQSIKVINAIIKKYGLPNDIIIELAREKNSKDAQKMINEMQKRNRQTNE
RIEEIIRTTGKENAKYLIEKIKLHDMQEGKCLYSLEAIPLEDLLNNPFNYEVDHIIPRSVSFDNSFNN
KVLVKQEENSKKGNRTPFQYLSSSDSKISYETFKKHILNLAKGKGRISKTKKEYLLEERDINRFSV
QKDFINRNLVDTRYATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYKEIFITPHQIKHIKDFK
DYKYSHRVDKKPNRELINDTLYSTRKDDKGNTLIVNNLNGLYDKDNDKLKKLINKSPEKLLMYH
HDPQTYQKLKLIMEQYGDEKNPLYKYYEETGNYLTKYSKKDNGPVIKKIKYYGNKLNAHLDITD
DYPNSRNKVVKLSLKPYRFDVYLDNGVYKFVTVKNLDVIKKENYYEVNSKCYEEAKKLKKISN
QAEFIASFYNNDLIKINGELYRVIGVNNDLLNRIEVNMIDITYREYLENMNDKRPPRIIKTIASKTQS
IKKYSTDILGNLYEVKSKKHPQIIKKGSGGSKRTADGSEFEPKKKRKV
SaKKH-ABE8e-dimer
(SEQ ID NO: 182)
MKRTADGSEFESPKKKRKVSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTDSGGSSG
GSSGSETPGTSESATPESSGGSSGGSSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGRVVFG
VRNSKRGAAGSLMNVLNYPGMNHRVEITEGILADECAALLCDFYRMPRQVFNAQKKAQSSINS
GGSSGGSSGSETPGTSESATPESSGGSSGGSGKRNYILGLAIGITSVGYGIIDYETRDVIDAGVRLFK
EANVENNEGRRSKRGARRLKRRRRHRIQRVKKLLFDYNLLTDHSELSGINPYEARVKGLSQKLSE
EEFSAALLHLAKRRGVHNVNEVEEDTGNELSTKEQISRNSKALEEKYVAELQLERLKKDGEVRG
SINRFKTSDYVKEAKQLLKVQKAYHQLDQSFIDTYIDLLETRRTYYEGPGEGSPFGWKDIKEWYE
MLMGHCTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIYQSSEDI
QEELTNLNSELTQEEIEQISNLKGYTGTHNLSLKAINLILDELWHTNDNQIAIFNRLKLVPKKVDLS
QQKEIPTTLVDDFILSPVVKRSFIQSIKVINAIIKKYGLPNDIIIELAREKNSKDAQKMINEMQKRNR
QTNERIEEIIRTTGKENAKYLIEKIKLHDMQEGKCLYSLEAIPLEDLLNNPFNYEVDHIIPRSVSFDN
SFNNKVLVKQEENSKKGNRTPFQYLSSSDSKISYETFKKHILNLAKGKGRISKTKKEYLLEERDIN
RFSVQKDFINRNLVDTRYATRGLMNLLRSYFRVNNLDVKVKSINGGFTSFLRRKWKFKKERNKG
YKHHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSPEKL
LMYHHDPQTYQKLKLIMEQYGDEKNPLYKYYEETGNYLTKYSKKDNGPVIKKIKYYGNKLNAH
LDITDDYPNSRNKVVKLSLKPYRFDVYLDNGVYKFVTVKNLDVIKKENYYEVNSKCYEEAKKL
KKISNQAEFIASFYNNDLIKINGELYRVIGVNNDLLNRIEVNMIDITYREYLENMNDKRPPRIIKTIA
SKTQSIKKYSTDILGNLYEVKSKKHPQIIKKGSGGSKRTADGSEFEPKKKRKV
CP1028-ABE8e
(SEQ ID NO: 183)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRNS
KRGAAGSLMNVLNYPGMNHRVEITEGILADECAALLCDFYRMPRQVFNAQKKAQSSINSGGSSG
GSSGSETPGTSESATPESSGGSSGGSEIGKATAKYFFYSNIMNFFKTEITLANGEIRKRPLIETNGET
GEIVWDKGRDFATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLTNLGAPAAFKYFDTTID
RKRYTSTKEVLDATLIHQSITGLYETRIDLSQLGGDGGSGGSGGSGGSGGSGGSGGMDKKYSIGL
AIGTNSVGWAVITDEYKVPSKKFKVLGNTDRHSIKKNLIGALLFDSGETAEATRLKRTARRRYTRR
KNRICYLQEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAWMTRKSEET
ITPWNFEEVVDKGASAQSFIERMTNFDKNLPNEKVLPKHSLLYEYFTVYNELTKVKYVTEGMRK
PAFLSGEQKKAIVDLLFKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHIANLAGSPAI
KKGILQTVKVVDELVKVMGRHKPENIVIEMARENQTTQKGQKNSRERMKRIEEGIKELGSQILKE
HPVENTQLQNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIKRQLVETR
QITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQSGGSKRTADGSEFEPKKKRKV
CP1028-ABE8e-dimer
(SEQ ID NO: 184)
MKRTADGSEFESPKKKRKVSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTDSGGSSG
GSSGSETPGTSESATPESSGGSSGGSSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIGRVVFG
VRNSKRGAAGSLMNVLNYPGMNHRVEITEGILADECAALLCDFYRMPRQVFNAQKKAQSSINS
GGSSGGSSGSETPGTSESATPESSGGSSGGSEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYKEVKKDLI
IKLPKYSLFELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNLGAPAAFKY
FDTTIDRKRYTSTKEVLDATLIHQSITGLYETRIDLSQLGGDGGSGGSGGSGGSGGSGGSGGMDK
KYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELLVKLNREDL
LRKQRTFDNGSIPHQIHLGELHAILRRQEDFYPFLKDNREKIEKILTFRIPYYVGPLARGNSRFAWM
TRKSEETITPWNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSLHEHIAN
LAGSPAIKKGILQTVKVVDELVKVMGRHKPENIVIEMARENQTTQKGQKNSRERMKRIEEGIKEL
GSQILKEHPVENTQLQNEKLYLYYLQNGRDMYVDQELDINRLSDYDVDHIVPQSFLKDDSIDNK
VLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQSGGSKRTADGSEFEPKKKRK
V
CP1041-ABE8e
(SEQ ID NO: 185)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRNS
KRGAAGSLMNVLNYPGMNHRVEITEGILADECAALLCDFYRMPRQVFNAQKKAQSSINSGGSSG
GSSGSETPGTSESATPESSGGSSGGSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYFDTTIDRKRYTSTKEVL
DATLIHQSITGLYETRIDLSQLGGDGGSGGSGGSGGSGGSGGSGGDKKYSIGLAIGTNSVGWAVIT
DEYKVPSKKFKVLGNTDRHSIKKNLIGALLFDSGETAEATRLKRTARRRYTRRKNRICYLQEIFSN
EMAKVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQILKEHPVENTQLQNEK
LYLYYLQNGRDMYVDQELDINRLSDYDVDHIVPQSFLKDDSIDNKVLTRSDKNRGKSDNVPSEE
VVKKMKNYWRQLLNAKLITQRKFDNLTKAERGGLSELDKAGFIKRQLVETRQITKHVAQILDSR
MNTKYDENDKLIREVKVITLKSKLVSDFRKDFQFYKVREINNYHHAHDAYLNAVVGTALIKKYP
KLESEFVYGDYKVYDVRKMIAKSEQEIGKATAKYFFYSSGGSKRTADGSEFEPKKKRKV
ABE8e(TadA-8e V82G)
(SEQ ID NO: 186)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRNS
KRGAAGSLMNVLNYPGMNHRVEITEGILADECAALLCDFYRMPRQVFNAQKKAQSSINSGGSSG
GSSGSETPGTSESATPESSGGSSGGSDKKYSIGLAIGTNSVGWAVITDEYKVPSKKFKVLGNTDRH
SIKKNLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDY
FKKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMYVDQELD
INRLSDYDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDSGGSKRTADGSEFEPKKKRKV
ABE8e(TadA-8e K20A, R21A)
(SEQ ID NO: 187)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGVRNS
KRGAAGSLMNVLNYPGMNHRVEITEGILADECAALLCDFYRMPRQVFNAQKKAQSSINSGGSSG
GSSGSETPGTSESATPESSGGSSGGSDKKYSIGLAIGTNSVGWAVITDEYKVPSKKFKVLGNTDRH
SIKKNLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDY
FKKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMYVDQELD
INRLSDYDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDSGGSKRTADGSEFEPKKKRKV
ABE8e(TadA-8e V106W)
(SEQ ID NO: 188)
MKRTADGSEFESPKKKRKVSEVEFSHEYWMRHALTLAKRARDEREVPVGAVLVLNNRVIGEGW
NRAIGLHDPTAHAEIMALRQGGLVMQNYRLIDATLYVTFEPCVMCAGAMIHSRIGRVVFGWRNS
KRGAAGSLMNVLNYPGMNHRVEITEGILADECAALLCDFYRMPRQVFNAQKKAQSSINSGGSSG
GSSGSETPGTSESATPESSGGSSGGSDKKYSIGLAIGTNSVGWAVITDEYKVPSKKFKVLGNTDRH
SIKKNLIGALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKDNRE
KIEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDY
FKKIECFDSVEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMYVDQELD
INRLSDYDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDSGGSKRTADGSEFEPKKKRKV
ABE8e-NRTH dimer editor: NLS, wtTadA, linker, TadA*, SpCas9-NRTH
(SEQ ID NO: 449)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSETPGTSESATPESSGGSSGGSSEVEFSHEYWMRHALTLAKRARDEREVPVG
AVLVLNNRVIGEGWNRAIGLHDPTAHAEIMALRQGGLVMQNYRLIDATLYVTFEPCVMCAGAMIHSRIG
RVVFGVRNSKRGAAGSLMNVLNYPGMNHRVEITEGILADECAALLCDFYRMPRQVFNAQKKAQSSINS
GGSSGGSSGSYTPGTSYSKYPYSSGGSSGGSDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDILRVNTEITKAPLSASM
VKRYDEHHQDLTLLKALVRQQLPEKYKEIFFDQSKNGYAGYIDGGASQEEFYKFIKPILEKMDGTEELL
VKLNREDLLRKQRTFDNGIIPHQIHLGELHAILRRQGDFYPFLKDNREKIEKILTFRIPYYVGPLARGNS
RFAWMTRKSEETITPWNFEEWDKGASAQSFIERMTNFDKNLPNEKVLPKHSLLYEYFTVYNELTKVKY
VTEGMRKPAFLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRLRYTGWGRLSRKLIN
GIRDKQSGKTILDFLKSDGFANRNFMQLIHDDSLTFKEDIQKAQVSCQGDSLHEHIANLAGSPAIKKGI
LQTVKVVDELIKVMGGHKPENIVIEMARENQTTQKGQKNSRERMKRIEEGIKELGSQILKEHPVENTQ
LQNEKLYLYYLQNGRDMYVDQELDINRLSDYDVDHIVPQSFLKDDSIENKVLTRSDKNRGKSDNVPSE
EVVKKMKNYWRQLLNAKLITQRKFDNLTKAERGGLSELDKAGFIKRQLAETRQITKHVAQILDSRMNT
KYDENDKLIREVKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEIVWDKGRDFA
TVRKVLSMPQVNIVKKTEVQTGGFSKESILPKGNSDKLIARKKDWDPKKYGGFNSPTVAYSVLVVAKVE
KGKSKKLKSVKELLGITIMERSSFEKNPIGFLEAKGYKEVKKDLIIKLPKYSLFELENGRKRMLASASVL
HKGNELALPSKYVNFLYLASHYEKLKGSSEDNKQKQLFVEQHKHYLDEIIEQISEFSKRVILADANLDKV
LSAYNKHRDKPIREQAENIIHLFTLTNLGASAAFKYFDTTIGRKLYTSTKEVLDATLIHQSITGLYETRIDLS
QLGGDSGGSKRTADGSEFEPKKKRKV
ABE8e-NRTH monomer editor: NLS, linker, TadA*, SpCas9-NRTH
(SEQ ID NO: 465)
MKRTADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSES
ATPESSGGSSGGSDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMVKRYDEHHQDLTLLK
ALVRQQLPEKYKEIFFDQSKNGYAGYIDGGASQEEFYKFIKPILEKMDGTEELLVKLNREDLLRKQRTF
DNGIIPHQIHLGELHAILRRQGDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVFEGMRKPAFLSGEQ
KKAIVDLLFKTNRKVTVKQLKEDYFKKIECFDSVEISGVEDRFNASLGTYHDLLKIIKDKDFLDNEENE
DILEDIVLTLTLFEDREMIEERLKTYAHLFDDKVMKQLKRLRYTGWGRLSRKLINGIRDKQSGKTILDFL
KSDGFANRNFMQLIHDDSLTFKEDIQKAQVSCQGDSLHEHIANLAGSPAIKKGILQTVKVVDELIKVMG
GHKPENIVIEMARENQTTQKGQKNSRERMKRIEEGIKELGSQILKEHPVENTQLQNEKLYLYYLQNGR
DMYVDQELDINRLSDYDVDHIVPQSFLKDDSIENKVLTRSDKNRGKSDNVPSEEVVKKMKNYWRQLLN
AKLITQRKFDNLTKAERGGLSELDKAGFIKRQLAETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRKVLSMPQVNIVKK
TEVQTGGFSKESILPKGNSDKLIARKKDWDPKKYGGFNSPTVAYSVLWAKVEKGKSKKLKSVKELLGI
TIMERSSFEKNPIGFLEAKGYKEVKKDLIIKLPKYSLFELENGRKRMLASASVLHKGNELALPSKYVNFL
YLASHYEKLKGSSEDNKQKQLFVEQHKHYLDEIIEQISEFSKRVILADANLDKVLSAYNKHRDKPIREQA
ENIIHLFTLTNLGASAAFKYFDTTIGRKLYTSTKEVLDATLIHQSITGLYETRIDLSQLGGDSGGSKRTAD
GSEFEPKKKRKV
ABE8e-SpyMac dimer editor: NLS, wtTadA, linker, TadA*, SpCas9-SpyMac
(SEQ ID NO: 450)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSETPGTSESATPESSGGSSGGSSEVEFSHEYWMRHALTLAKRARDEREVPVGAV
LVLNNRVIGEGWNRAIGLHDPTAHAEIMALRQGGLVMQNYRLIDATLYVFFEPCVMCAGAMIHSRIGRV
VFGVRNSKRGAAGSLMNVLNYPGMNHRVEITEGILADECAALLCDFYRMPRQVFNAQKKAQSSINSGG
SSGGSSGSETPGTSESATPESSGGSSGGSDKKYSIGLAIGTNSVGWAVITDEYKVPSKKFKVLGNTDRH
SIKKNLIGALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEIVWDKGRDFATV
RKVLSMPQVNTVKKTEIQTVGQNGGLFDDNPKSPLEVFPSKLVPLKKELNPKKYGGYQKPTTAYPVLLI
TDTKQLIPISVMNKKQFEQNPVKFLRDRGYQQVGKNDFIKLPKYTLVDIGDGIKRLWASSKEIHKGNQL
WSKKSQILLYHAHHLDSDLSNDYLQNHNQQFDVLFNEIISFSKKCKLGKEHIQKIENVYSNKKNSASIE
ELAESFIKLLGFTQLGATSPFNFLGVKLNQKQYKGKKDYILPCTEGTLIRQSITGLYETRVDLSKIGEDS
GGSKRTADGSEFEPKKKRKV
ABE8e-SpyMac editor: NLS, linker, TadA*, SpCas9-SpyMac
(SEQ ID NO: 466)
MKRTADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSE
SATPESSGGSSGGSDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFLSGEQK
KAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDHIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLSMPQVNIVKKT
EIQTVGQNGGLFDDNPKSPLEVTPSKLVPLKKELNPKKYGGYQKPTTAYPVLLITDTKQLIPISVMNKK
QFEQNPVKFLRDRGYQQVGKNDFIKLPKYTLVDIGDGIKRLWASSKEIHKGNQLWSKKSQILLYHAHH
LDSDLSNDYLQNHNQQFDVLFNEIISFSKKCKLGKEHIQKIENVYSNKKNSASIEELAESFIKLLGFTQL
GATSPFNFLGVKLNQKQYKGKKDYILPCTEGTLIRQSITGLYETRVDLSKIGEDSGGSKRTADGSEFEP
KKKRKV
ABE8e-VRQR-CP1041 dimer: NLS, wtTadA, linker, TadA*, SpCas9-VRQR-CP1041
(SEQ ID NO: 451)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSETPGTSESAATESSGGSSGGSSEVEFSHEYWMRHALTLAKRARDEREVPVG
AVLVLNNRVIGEGWNRAIGLHDPTAHAEIMALRQGGLVMQNYRLIDATLYVTFEPCVMCAGAMIHSRIG
RVVFGVRNSKRGAAGSLMNVLNYPGMNHRVEITEGILADECAALLCDFYRMPRQVFNAQKKAQSSINS
GGSSGGSSGSETPGTSESATPESSGGSSGGSNIMNFFKTEITLANGEIRKRPLIETNGETGEIVWDKGR
DFATVRKVLSMPQVNIVKKTEVQTGGFSKESIRPKRNSDKLIARKKDWDPKKYGGFVSPTVAYSVLWA
KVEKGKSKKLKSVKELLGITIMERSSFEKNPIDFLEAKGYKEVKKDLIIKLPKYSLFELENGRKRMLASA
RFLQKGNELALPSKYVNFLYLASHYEKLKGSPEDNEQKQLFVEQHKHYLDEIIEQISEFSKRVILADANL
DKVLSAYNKHRDKPIREQAENIIHLFTLTNLGAPRAFKYFDTTIDRKVYRSTKEVLDATLIHQSITGLYET
RIDLSQLGGDGGSGGSGGSGGSGGSGGSGGDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTVYNELTKVKY
VTEGMRKPAFLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKNRGKSDNVPS
EEVVKKMKNYWRQLLNAKLITQRKFDNLTKAERGGLSELDKAGFIKRQLVETRQITKHVAQILDSRMN
TKYDENDKLIREVKVITLKSKLVSDFRKDFQFYKVREINNYHHAHDAYLNAVVGTALIKKYPKLESEFVY
GDYKVYDVRKMIAKSEQEIGKATAKYFFYSSGGSKRTADGSEEEPKKKRKV
ABE8e-VRQR-CP1041 monomer: NLS, linker, TadA* SpCas9-VRQR-CP1041
(SEQ ID NO: 467)
MKRTADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSES
ATPESSGGSSGGSNIMNFFKTEITLANGEIRKRPLIETNGETGEIVWDKGRDFATVRKVLSMPQVNIVKK
TEVQTGGFSKESIRPKRNSDKLIARKKDWDPKKYGGFVSPTVAYSVLVVAKVEKGKSKKLKSVKELLGIT
IMERSSFEKNPIDFLEAKGYKEVKKDLIIKLPKYSLFELENGRKRMLASARFLQKGNELALPSKYVNFLY
LASHYEKLKGSPEDNEQKQLFVEQHKHYLDEIIEQISEFSKRVILADANLDKVLSAYNKHRDKPIREQAE
NIIHLFTLTNLGAPRAFKYFDTTIDRKVYRSTKEVLDATLIHQSITGLYETRIDLSQLGGDGGSGGSGGSG
GSGGSGGSGGDKKYSIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDEHHQDLTLLKALVRQ
QLPEKYKEIFFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDILEDI
VLTLTLFEDREMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYV
DQELDINRLSDYDVDHIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVRKMIAKSEQEIGK
ATAKYFFYSSGGSKRTADGSEFEPKKKRKV
ABE8e-SaCas9 dimer editor: NLS, wtTadA, linker, TadA*, SaCas9
(SEQ ID NO: 452)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSETPGTSESATPESSGGSSGGSSEVEFSHEYWMRHALTLAKRARDEREVPVG
AVLVLNNRVIGEGWNRAIGLHDPTAHAEIMALRQGGLVMQNYRLIDATLYVTFEPCVMCAGAMIHSRIG
RVVFGVRNSKRGAAGSLMNVLNYPGMNHRVEITEGILADECAALLCDFYRMPRQVFNAQKKAQSSINS
GGSSGGSSGSETPGTSESATPESSGGSSGGSGKRNYILGLAIGITSVGYGIIDYETRDVIDAGVRLFKEA
NVENNEGRRSKRGARRLKRRRRHRIQRVKKLLFDYNLLTDHSELSGINPYEARVKGLSQKLSEEEFSAA
LLHLAKRRGVHNVNEVEEDTGNELSTKEQISRNSKALEEKYVAELQLERLKKDGEVRGSINRFKTSDYV
KEAKQLLKVQKAYHQLDQSFIDTYIDLLETRRTYYEGPGEGSPFGWKDIKEWYEMLMGHCTYFPEELR
SVKYAYNADLYNALNDLNNLVITRDENEKLEYYEKFQIIENVFKQKKKPTLKQIAKEILVNEEDIKGYRVT
STGKPEFTNLKVYHDIKDITARKEIIENAELLDQIAKILTIYQSSEDIQEELTNLNSELTQEEIEQISNLKGY
TGTHNLSLKAINLILDELWHTNDNQIAIFNRLKLVPKKVDLSQQKEIPTTLVDDFILSPVVKRSFIQSIKVI
NAIIKKYGLPNDIIIELAREKNSKDAQKMINEMQKRNRQTNERIEEIIRTTGKENAKYLIEKIKLHDMQE
GKCLYSLEAIPLEDLLNNPFNYEVDHIIPRSVSFDNSFNNKVLVKQEENSKKGNRTPFQYLSSSDSKISYE
TFKKHILNLAKGKGRISKTKKEYLLEERDINRFSVQKDFINRNLVDTRYATRGLMNLLRSYFRVNNLDVK
VKSINGGFTSFLRRKWKFKKERNKGYKHHAEDALIIANADFIFKEWKKLDKAKKVMENQMFEEKQAE
SMPEIETEQEYKEIFITPHQIKHIKDFKDYKYSHRVDKKPNRELINDTLYSTRKDDKGNTLIVNNLNGLY
DKDNDKLKKLINKSPEKLLMYHHDPQTYQKLKLIMEQYGDEKNPLYKYYEETGNYLTKYSKKDNGPVI
KKIKYYGNKLNAHLDITDDYPNSRNKWKLSLKPYRFDVYLDNGVYKFVTVKNLDVIKKENYYEVNSKC
YEEAKKLKKISNQAEFIASFYNNDLIKINGELYRVIGVNNDLLNRIEVNMIDITYREYLENMNDKRPPRIIK
TIASKTQSIKKYSTDILGNLYEVKSKKHPQIIKKGSGGSKRTADGSEFEPKKKRKV
ABE8e-SaCas9 monomer editor: NLS, linker, TadA*, SaCas9
(SEQ ID NO: 468)
MKRTADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSES
ATPESSGGSSGGSGKRNYILGLAIGITSVGYGIIDYETRDVIDAGVRLFKEANVENNEGRRSKRGARRLK
RRRRHRIQRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQKAYHQLDQ
SFIDTYIDLLETRRTYYEGPGEGSPFGWKDIKEWYEMLMGHCTYFPEELRSVKYAYNADLYNALNDLNN
LVITRDENEKLEYYEKFQIIENVFKQKKKPTLKQIAKEILVNEEDIKGYRVTSTGKPEFTNLKVYHDIKDI
TARKEIIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIIIELARE
KNSKDAQKMINEMQKRNRQTNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKISYETFKKHILNLAKGKGRISKT
KKEYLLEERDINRFSVQKDFINRNLVDTRYATRGLMNLLRSYFRVNNLDVKVKSINGGFTSFLRRKWKF
KKERNKGYKHHAEDALIIANADFIFKEWKKLDKAKKVMENQMFEEKQAESMPEIETEQEYKEIFITPH
QIKHIKDFKDYKYSHRVDKKPNRELINDTLYSTRKDDKGNTLIVNNLNGLYDKDNDKLKKLINKSPEKL
LMYHHDPQTYQKLKLIMEQYGDEKNPLYKYYEETGNYLTKYSKKDNGPVIKKIKYYGNKLNAHLDITD
DYPNSRNKVVKLSLKPYRFDVYLDNGVYKFVTVKNLDVIKKENYYEVNSKCYEEAKKLKKISNQAEFIAS
FYNNDLIKINGELYRVIGVNNDLLNRIEVNMIDITYREYLENMNDKRPPRIIKTIASKTQSIKKYSTDILGN
LYEVKSKKHPQIIKKGSGGSKRTADGSEFEPKKKRKV
ABE8e-NRCH dimer editor: NLS, wtTadA, linker, TadA*, SpCas9-NRCH
(SEQ ID NO: 453)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSETPGTSESATPESSGGSSGGSSEVEFSHEYWMRHALTLAKRARDEREVPVG
AVLVLNNRVIGEGWNRAIGLHDPTAHAEIMALRQGGLVMQNYRLIDATLYVTFEPCVMCAGAMIHSRIG
RVVFGVRNSKRGAAGSLMNVLNYPGMNHRVEITEGILADECAALLCDFYRMPRQVFNAQKKAQSSINS
GGSSGGSSGSETPGTSESATPESSGGSSGGSDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDILRVNTEITKAPLSASM
VKRYDEHHQDLTLLKALVRQQLPEKYKEIFFDQSKNGYAGYIDGGASQEEFYKFIKPILEKMDGTEELL
VKLNREDLLRKQRTFDNGIIPHQIHLGELHAILRRQGDFYPFLKDNREKIEKILTFRIPYYVGPLARGNS
RFAWMTRKSEETITPWNFEEVVDKGASAQSFIERMTNFDKNLPNEKVLPKHSLLYEYFTVYNELTKVKY
VTEGMRKPAFLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRLRYTGWGRLSRKLIN
GIRDKQSGKTILDFLKSDGFANRNFMQLIHDDSLTFKEDIQKAQVSCQGDSLHEHIANLAGSPAIKKGI
LQTVKVVDELIKVMGGHKPENIVIEMARENQTTQKGQKNSRERMKRIEEGIKELGSQILKEHPVENTQ
LQNEKLYLYYLQNGRDMYVDQELDINRLSDYDVDHIVPQSFLKDDSIENKVLTRSDKNRGKSDNVPSE
EWKKMKNYWRQLLNAKLITQRKFDNLTKAERGGLSELDKAGFIKRQLAETRQITKHVAQILDSRMNT
KYDENDKLIREVKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEIVWDKGRDFA
TVRKVLSMPQVNIVKKTEVQTGGFSKESILPKGNSDKLIARKKDWDPKKYGGFNSPTVAYSVLVVAKVE
KGKSKKLKSVKELLGITIMERSSFEKNPIDFLEAKGYKEVKKDLIIKLPKYSLFELENGRKRMLASAGVL
QKGNELALPSKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTINRKQYNTTKEVLDATLIRQSITGLYETRID
LSQLGGDSGGSKRTADGSEFEPKKKRKV
ABE8e-NRCH monomer editor: NLS, linker, TadA*, SpCas9-NRCH
(SEQ ID NO: 469)
MKRTADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSES
ATPESSGGSSGGSDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMVKRYDEHHQDLTLLK
ALVRQQLPEKYKEIFFDQSKNGYAGYIDGGASQEEFYKFIKPILEKMDGTEELLVKLNREDLLRKQRTF
DNGIIPHQIHLGELHAILRRQGDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVFEGMRKPAFLSGEQ
KKAIVDLLFKTNRKVTVKQLKEDYFKKIECFDSVEISGVEDRFNASLGTYHDLLKHKDKDFLDNEENE
DILEDIVLTLTLFEDREMIEERLKTYAHLFDDKVMKQLKRLRYTGWGRLSRKLINGIRDKQSGKTILDFL
KSDGFANRNFMQLIHDDSLTFKEDIQKAQVSCQGDSLHEHIANLAGSPAIKKGILQTVKWDELIKVMG
GHKPENIVIEMARENQTTQKGQKNSRERMKRIEEGIKELGSQILKEHPVENTQLQNEKLYLYYLQNGR
DMYVDQELDINRLSDYDVDHIVPQSFLKDDSIENKVLTRSDKNRGKSDNVPSEEVVKKMKNYWRQLLN
AKLITQRKFDNLTKAERGGLSELDKAGFIKRQLAETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRKVLSMPQVNIVKK
TEVQTGGFSKESILPKGNSDKLIARKKDWDPKKYGGFNSPTVAYSVLVVAKVEKGKSKKLKSVKELLGI
TIMERSSFEKNPIDFLEAKGYKEVKKDLIIKLPKYSLFELENGRKRMLASAGVLQKGNELALPSKYVNFL
YLASHYEKLKGSPEDNEQKQLFVEQHKHYLDEIIEQISEFSKRVILADANLDKVLSAYNKHRDKPIREQA
ENIIHLFTLTNLGAPAAFKYFDTTINRKQYNTTKEVLDATLIRQSITGLYETRIDLSQLGGDSGGSKRTAD
GSEFEPKKKRKV
ABE8e-NRRH dimer editor: NLS, wtTadA, linker, TadA*, SpCas9-NRRH
(SEQ ID NO: 454)
MKRTADGSEFESPKKKRKVSEVEFSHEYWMRHALTLAKRAWDEREVPVGAVLVHNNRVIGEGWNRPI
GRHDPTAHAEIMALRQGGLVMQNYRLIDATLYVTLEPCVMCAGAMIHSRIGRVVFGARDAKTGAAG
SLMDVLHHPGMNHRVEITEGILADECAALLSDFFRMRRQEIKAQKKAQSSTDSGGSSGGSSGSETPGT
SESATPESSGGSSGGSSEVEFSHEYWMRHALTLAKRARDEREVPVGAVLVLNNRVIGEGWNRAIGLHDPTAHAEI
MALRQGGLVMQNYRLIDATLYVTFEPCVMCAGAMIHSRIGRVVFGVRNSKRGAAGSLMNVLNYPGMNHRVEITE
GILADECAALLCDFYRMPRQVFNAQKKAQSSINSGGSSGGSSGSEYPGYSESAFPESSGGSSGGSDKKYSIGLAIG
TNSVGWAVITDEYKVPSKKFKVLGNTDRHSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILLSDILRVNTEITKAPLSAS
MVKRYDEHHQDLTLLKALVRQQLPEKYKEIFFDQSKNGYAGYIDGGASQEEFYKFIKPILEKMDGTEELLVKLNR
EDLLRKQRTFDNGIIPHQIHLGELHAILRRQGDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDILEDIVLTLTLF
EDREMIEERLKTYAHLFDDKVMKQLKRLRYTGWGRLSRKLINGIRDKQSGKTILDFLKSDGFANRNFMQLIHDDS
LTFKEDIQKAQVSCQGDSLHEHIANLAGSPAIKKGILQTVKWDELIKVMGGHKPENIVIEMARENQTTQKGQKNS
RERMKRIEEGIKELGSQILKEHPVENTQLQNEKLYLYYLQNGRDMYVDQELDINRLSDYDVDHIVPQSFLKDDSIE
NKVLTRSDKNRGKSDNVPSEEVVKKMKNYWRQLLNAKLITQRKFDNLTKAERGGLSELDKAGFIKRQLAETRQIT
KHVAQILDSRMNTKYDENDKLIREVKVITLKSKLVSDFRKDFQFYKVREINNYHHAHDAYLNAVVGTALIKKYPKLE
SEFVYGDYKVYDVRKMIAKSEQEIGKATAKYFFYSNIMNFFKTEITLANGEIRKRPLIETNGETGEIVWDKGRDFAT
VRKVLSMPQVNIVKKTEVQTGGFSKESILPKGNSDKLIARKKDWDPKKYGGFNSPTAAYSVLVVAKVEKGKSKKLK
SVKELLGITIMERSSFEKNPIGFLEAKGYKEVKKDLIIKLPKYSLFELENGRKRMLASAGVLHKGNELALPSKYVNF
LYLASHYEKLKGSPEDNEQKQLFVEQHKHYLDEIIEQISEFSKRVILADANLDKVLSAYNKHRDKPIREQAENIIHLF
TLTNLGVPAAFKYFDTTIDKKRYTSTKEVLDATLIHQSITGLYETRIDLSQLGGDSGGSKRTADGSEFEPKKKRKV
ABE8e-NRRH monomer editor NLS, linker, TadA*, SpCas9-NRRF1
(SEQ ID NO: 470)
MKRPADGSEEESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRWFGVRNSKRGAAGSL
MNVLNYPGMNHRVEITEGILADECAALLCDFYRMPRQVFNAQKKAQSSINSGGSSGGSSGSETPGTSE
SAPPESSGGSSGGSDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMVKRYDEHHQDLTLLK
ALVRQQLPEKYKEIFFDQSKNGYAGYIDGGASQEEFYKFIKPILEKMDGTEELLVKLNREDLLRKQRTF
DNGIIPHQIHLGELHAILRRQGDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVFEGMRKPAFLSGEQ
KKAIVDLLFKTNRKVTVKQLKEDYFKKIECFDSVEISGVEDRFNASLGTYHDLLKIIKDKDFLDNEENE
DILEDIVLTLTLFEDREMIEERLKTYAHLFDDKVMKQLKRLRYTGWGRLSRKLINGIRDKQSGKTILDFL
KSDGFANRNFMQLIHDDSLTFKEDIQKAQVSCQGDSLHEHIANLAGSPAIKKGILQTVKVVDELIKVMG
GHKPENIVIEMARENQTTQKGQKNSRERMKRIEEGIKELGSQILKEHPVENTQLQNEKLYLYYLQNGR
DMYVDQELDINRLSDYDVDHIVPQSFLKDDSIENKVLTRSDKNRGKSDNVPSEEVVKKMKNYWRQLLN
AKLITQRKFDNLTKAERGGLSELDKAGFIKRQLAETRQITKHVAQILDSRMNTKYDENDKLIREVKVITL
KSKLVSDFRKDFQFYKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRKVLSMPQVNIVKK
TEVQTGGFSKESILPKGNSDKLIARKKDWDPKKYGGFNSPTAAYSVLWAKVEKGKSKKLKSVKELLGI
TIMERSSFEKNPIGFLEAKGYKEVKKDLIIKLPKYSLFELENGRKRMLASAGVLHKGNELALPSKYVNFL
YLASHYEKLKGSPEDNEQKQLFVEQHKHYLDEIIEQISEFSKRVILADANLDKVLSAYNKHRDKPIREQA
ENIIHLFTLTNLGVPAAFKYFDTTIDKKRYTSTKEVLDATLIHQSITGLYETRIDLSQLGGDSGGSKRTAD
GSEFEPKKKRKV
SaKKH-ABE8e dimer editor: NLS, wtTadA, linker, TadA*, SaKKH
(SEQ ID NO: 455)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSEYPGYSESAYPESSGGSSGGSSEVEFSHEYWMRHALTLAKRARDEREVPVGAV
LVLNNRVIGEGWNRAIGLHDPTAHAEIMALRQGGLVMQNYRLIDATLYVTFEPCVMCAGAMIHSRIGRV
VFGVRNSKRGAAGSLMNVLNYPGMNHRVEITEGILADECAALLCDFYRMPRQVFNAQKKAQSSINSGG
SSGGSSGSETPGTSESATPESSGGSSGGSGKRNYILGLAIGITSVGYGIIDYETRDVIDAGVRLFKEANVE
NNEGRRSKRGARRLKRRRRHRIQRVKKLLFDYNLLTDHSELSGINPYEARVKGLSQKLSEEEFSAALLHL
AKRRGVHNVNEVEEDTGNELSTKEQISRNSKALEEKYVAELQLERLKKDGEVRGSINRFKTSDYVKEAK
QLLKVQKAYHQLDQSFIDTYIDLLETRRTYYEGPGEGSPFGWKDIKEWYEMLMGHCTYFPEELRSVKY
AYNADLYNALNDLNNLVITRDENEKLEYYEKFQIIENVFKQKKKPTLKQIAKEILVNEEDIKGYRVFSTG
KPEFTNLKVYHDIKDITARKEIIENAELLDQIAKILTIYQSSEDIQEELTNLNSELTQEEIEQISNLKGYTGT
HNLSLKAINLILDELWHTNDNQIAIFNRLKLVPKKVDLSQQKEIPTTLVDDFILSPVVKRSFIQSIKVINAII
KKYGLPNDIIIELAREKNSKDAQKMINEMQKRNRQTNERIEEIIRTTGKENAKYLIEKIKLHDMQEGKCL
YSLEAIPLEDLLNNPFNYEVDHIIPRSVSFDNSFNNKVLVKQEENSKKGNRTPFQYLSSSDSKISYETFKK
HILNLAKGKGRISKTKKEYLLEERDINRFSVQKDFINRNLVDTRYATRGLMNLLRSYFRVNNLDVKVKSI
NGGFTSFLRRKWKFKKERNKGYKHHAEDALIIANADFIFKEWKKLDKAKKVMENQMFEEKQAESMP
EIETEQEYKEIFITPHQIKHIKDFKDYKYSHRVDKKPNRKLINDTLYSTRKDDKGNTLIVNNLNGLYDKD
NDKLKKLINKSPEKLLMYHHDPQTYQKLKLIMEQYGDEKNPLYKYYEETGNYLTKYSKKDNGPVIKKI
KYYGNKLNAHLDITDDYPNSRNKWKLSLKPYRFDVYLDNGVYKFVTVKNLDVIKKENYYEVNSKCYEE
AKKLKKISNQAEFIASFYKNDLIKINGEEYRVIGVNNDLLNRIEVNMIDITYREYLENMNDKRPPHIIKTIA
SKTQSIKKYSTDILGNLYEVKSKKHPQIIKKGSGGSKRTADGSEFEPKKKRKV
SaKKH-ABE8e monomer editor: NLS, linker, TadA*, SaKKH
(SEQ ID NO: 471)
MKRTADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSE
SKPPESSGGSSGGSGKRNYILGLAIGITSVGYGIIDYETRDVIDAGVRLFKEANVENNEGRRSKRGARRL
KRRRRHRIQRVKKLLFDYNLLTDHSELSGINPYEARVKGLSQKLSEEEFSAALLHLAKRRGVHNVNEVE
EDTGNELSTKEQISRNSKALEEKYVAELQLERLKKDGEVRGSINRFKTSDYVKEAKQLLKVQKAYHQLD
QSFIDTYIDLLETRRTYYEGPGEGSPFGWKDIKEWYEMLMGHCTYFPEELRSVKYAYNADLYNALNDLN
NLVITRDENEKLEYYEKFQIIENVFKQKKKPTLKQIAKEILVNEEDIKGYRVFSTGKPEFTNLKVYHDIKD
ITARKEIIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIIIELARE
KNSKDAQKMINEMQKRNRQTNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKISYETFKKHILNLAKGKGRISKT
KKEYLLEERDINRFSVQKDFINRNLVDTRYATRGLMNLLRSYFRVNNLDVKVKSINGGFTSFLRRKWKF
KKERNKGYKHHAEDALIIANADFIFKEWKKLDKAKKVMENQMFEEKQAESMPEIETEQEYKEIFITPH
QIKHIKDFKDYKYSHRVDKKPNRKLINDTLYSTRKDDKGNTLIVNNLNGLYDKDNDKLKKLINKSPEKL
LMYHHDPQTYQKLKLIMEQYGDEKNPLYKYYEETGNYLTKYSKKDNGPVIKKIKYYGNKLNAHLDITD
DYPNSRNKVVKLSLKPYRFDVYLDNGVYKFVTVKNLDVIKKENYYEVNSKCYEEAKKLKKISNQAEFIAS
FYKNDLIKINGELYRVIGVNNDLLNRIEVNMIDITYREYLENMNDKRPPHIIKTIASKTQSIKKYSTDILGN
LYEVKSKKHPQIIKKGSGGSKRTADGSEFEPKKKRKV
ABE8e-NG dimer editor: NLS, wtTadA, linker, TadA*, SpCas9-NG
(SEQ ID NO: 456)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSEYPGYSESAYPESSGGSSGGSSEVEFSHEYWMRHALTLAKRARDEREVPVGAV
LVLNNRVIGEGWNRAIGLHDPTAHAEIMALRQGGLVMQNYRLIDATLYVTFEPCVMCAGAMIHSRIGRV
VFGVRNSKRGAAGSLMNVLNYPGMNHRVEITEGILADECAALLCDFYRMPRQVFNAQKKAQSSINSGG
SSGGSSGSETPGTSESATPESSGGSSGGSDKKYSIGLAIGTNSVGWAVITDEYKVPSKKFKVLGNTDRH
SIKKNLIGALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAWGTALIKKYPKLESEFVYGDY
KVYDVRKMIAKSEQEIGKATAKYFFYSNIMNFFKTEITLANGEIRKRPLIETNGETGEIVWDKGRDFATV
RKVLSMPQVNIVKKTEVQTGGFSKESIRPKRNSDKLIARKKDWDPKKYGGFVSPTVAYSVLVVAKVEKG
KSKKLKSVKELLGITIMERSSFEKNPIDFLEAKGYKEVKKDLIIKLPKYSLFELENGRKRMLASARFLQK
GNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPRAFKYFDTTIDRKVYRSTKEVLDATLIHQSITGLYETRIDLSQ
LGGDSGGSKRTADGSEFEPKKKRKV
ABE8e-NG monomer editor: NLS, linker, TadA*, SpCas9-NG (“NG-ABE8e”)
(SEQ ID NO: 472)
MKRTADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSE
SATPESSGGSSGGSDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFLSGEQK
KAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDHIVPQSFLKDDSIDNKVLTRSDKNRGKSDNVPSEEWKKMKNYWRQLLNA
KLITQRKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLSMPQVNIVKKT
EVQTGGFSKESIRPKRNSDKLIARKKDWDPKKYGGFVSPTVAYSVLWAKVEKGKSKKLKSVKELLGITI
MERSSFEKNPIDFLEAKGYKEVKKDLIIKLPKYSLFELENGRKRMLASARFLQKGNELALPSKYVNFLYL
ASHYEKLKGSPEDNEQKQLFVEQHKHYLDEIIEQISEFSKRVILADANLDKVLSAYNKHRDKPIREQAE
NIIHLFTLTNLGAPRAFKYFDTTIDRKVYRSTKEVLDATLIHQSITGLYETRIDLSQLGGDSGGSKRTADG
SEFEPKKKRKV
ABE8e-CP1041 dimer editor: NLS, wtTadA, linker, TadA*, CP1041
(SEQ ID NO: 457)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSEYPGYSESAYPESSGGSSGGSSEVEFSHEYWMRHALTLAKRARDEREVPVGAV
LVLNNRVIGEGWNRAIGLHDPTAHAEIMALRQGGLVMQNYRLIDATLYVTFEPCVMCAGAMIHSRIGRV
VFGVRNSKRGAAGSLMNVLNYPGMNHRVEITEGILADECAALLCDFYRMPRQVFNAQKKAQSSINSGG
SSGGSSGSETPGTSESATPESSGGSSGGSNIMNFFKTEITLANGEIRKRPLIETNGETGEIVWDKGRDFA
TVRKVLSMPQVNIVKKTEVQTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPKYSLFELENGRKRMLASAGEL
QKGNELALPSKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTSTKEVLDATLIHQSITGLYETRID
LSQLGGDGGSGGSGGSGGSGGSGGSGGDKKYSIGLAIGTNSVGWAVITDEYKVPSKKFKVLGNTDRHS
IKKNLIGALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNASLGTYHDLLKn
KDKDFLDNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAWGTALIKKYPKLESEFVYGDY
KVYDVRKMIAKSEQEIGKATAKYFFYSSGGSKRTADGSEFEPKKKRKV
ABE8e-CP1041 monomer editor: NLS, linker, TadA*, CP1041
(SEQ ID NO: 473)
MKRTADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSE
SATPESSGGSSGGSNIMNFFKTEITLANGEIRKRPLIETNGETGEIVWDKGRDFATVRKVLSMPQVNIVK
KTEVQTGGFSKESILPKRNSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHRDKPIREQ
AENIIHLFTLTNLGAPAAFKYFDTTIDRKRYTSTKEVLDATLIHQSITGLYETRIDLSQLGGDGGSGGSGG
SGGSGGSGGSGGDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAEDAKLQLSKD
TYDDDLDNLLAQIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKSEETITPWNF
EEVVDKGASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDHIVPQSFLKDDSIDNKVLTRSDKNRGKSDNVPSEEWKKMKNYWRQLLNA
KLITQRKFDNLTKAERGGLSELDKAGFIKRQLVETRQITKHVAQILDSRMNTKYDENDKLIREVKVITLK
SKLVSDFRKDFQFYKVREINNYHHAHDAYLNAWGTALIKKYPKLESEFVYGDYKVYDVRKMIAKSEQE
IGKATAKYFFYSSGGSKRTADGSEFEPKKKRKV
ABE8e-CP1028 dimer editor: NLS, wtTadA, linker, TadA*, CP1028
(SEQ ID NO: 458)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSETPGTSESATPESSGGSSGGSSEVEFSHEYWMRHALTLAKRARDEREVPVGAV
LVLNNRVIGEGWNRAIGLHDPTAHAEIMALRQGGLVMQNYRLIDATLYVTFEPCVMCAGAMIHSRIGRV
VFGVRNSKRGAAGSLMNVLNYPGMNHRVEITEGILADECAALLCDFYRMPRQVFNAQKKAQSSINSGG
SSGGSSGSETPGTSESATPESSGGSSGGSEIGKATAKYFFYSNIMNFFKTEITLANGEIRKRPLIETNGET
GEIVWDKGRDFATVRKVLSMPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYFDTTIDRKRYTSTKEVLDATLI
HQSITGLYETRIDLSQLGGDGGSGGSGGSGGSGGSGGSGGMDKKYSIGLAIGTNSVGWAVITDEYKVPS
KKFKVLGNTDRHSIKKNLIGALLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEFYKFIKPI
LEKMDGTEELLVKLNREDLLRKQRTFDNGSIPHQIHLGELHAILRRQEDFYPFLKDNREKIEKILTFRIP
YYVGPLARGNSRFAWMTRKSEETITPWNFEEWDKGASAQSFIERMTNFDKNLPNEKVLPKHSLLYEYF
TVYNELTKVKYVTEGMRKPAFLSGEQKKAIVDLLFKTNRKVTVKQLKEDYFKKIECFDSVEISGVEDRF
NASLGTYHDLLKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKVLTRSDKN
RGKSDNVPSEEWKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAWGTALIKKY
PKLESEFVYGDYKVYDVRKMIAKSEQSGGSKRTADGSEFEPKKKRKV
ABE8e-CP1028 monomer editor NLS, linker, TadA*, CP1028
(SEQ ID NO: 474)
MKRTADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSE
SATPESSGGSSGGSEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFDSPTVAYSVLWAKVEKG
KSKKLKSVKELLGITIMERSSFEKNPIDFLEAKGYKEVKKDLIIKLPKYSLFELENGRKRMLASAGELQK
GNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRIDLSQ
LGGDGGSGGSGGSGGSGGSGGSGGMDKKYSIGLAIGTNSVGWAVITDEYKVPSKKFKVLGNTDRHSIK
KNLIGALLFDSGETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYVGPLARGNSRFAW
MTRKSEETITPWNFEEWDKGASAQSFIERMTNFDKNLPNEKVLPKHSLLYEYFTVYNELTKVKYVTEG
MRKPAFLSGEQKKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAGSPAIKKGILQTV
KVVDELVKVMGRHKPENIVIEMARENQTTQKGQKNSRERMKRIEEGIKELGSQILKEHPVENTQLQNE
KLYLYYLQNGRDMYVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEFVYGDYKV
YDVRKMIAKSEQSGGSKRTADGSEFEPKKKRKV
ABE8e-VRQR dimer editor: NLS, wtTadA, linker, TadA*, SpCas9-VRQR
(SEQ ID NO: 460)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSEYPGYSESAYPESSGGSSGGSSEVEFSHEYWMRHALTLAKRARDEREVPVGAV
LVLNNRVIGEGWNRAIGLHDPTAHAEIMALRQGGLVMQNYRLIDATLYVTFEPCVMCAGAMIHSRIGRV
VFGVRNSKRGAAGSLMNVLNYPGMNHRVEITEGILADECAALLCDFYRMPRQVFNAQKKAQSSINSGG
SSGGSSGSETPGTSESATPESSGGSSGGSDKKYSIGLAIGTNSVGWAVITDEYKVPSKKFKVLGNTDRH
SIKKNLIGALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGGFVSPTVAYSVLVVAKVEKG
KSKKLKSVKELLGITIMERSSFEKNPIDFLEAKGYKEVKKDLIIKLPKYSLFELENGRKRMLASARELQK
GNELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKQYRSTKEVLDATLIHQSITGLYETRIDLSQ
LGGDSGGSKRTADGSEFEPKKKRKV
ABE8e-VRQR monomer editor: NLS, linker, TadA*, SpCas9-VRQR
(SEQ ID NO: 476)
MKRTADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSE
SATPESSGGSSGGSDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFLSGEQK
KAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDHIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLSMPQVNIVKKT
EVQTGGFSKESILPKRNSDKLIARKKDWDPKKYGGFVSPTVAYSVLWAKVEKGKSKKLKSVKELLGITI
MERSSFEKNPIDFLEAKGYKEVKKDLIIKLPKYSLFELENGRKRMLASARELQKGNELALPSKYVNFLYL
ASHYEKLKGSPEDNEQKQLFVEQHKHYLDEIIEQISEFSKRVILADANLDKVLSAYNKHRDKPIREQAE
NIIHLFTLTNLGAPAAFKYFDTTIDRKQYRSTKEVLDATLIHQSITGLYETRIDLSQLGGDSGGSKRTADG
SEFEPKKKRKV
ABE8e-NG-CP1041 dimer editor: NLS, wtTadA, linker, TadA*, SpCas9-NG-CP1041
(SEQ ID NO: 461)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSETPGTSESATPESSGGSSGGSSEVEFSHEYWMRHALTLAKRARDEREVPVGAV
LVLNNRVIGEGWNRAIGLHDPTAHAEIMALRQGGLVMQNYRLIDATLYVTFEPCVMCAGAMIHSRIGRV
VFGVRNSKRGAAGSLMNVLNYPGMNHRVEITEGILADECAALLCDFYRMPRQVFNAQKKAQSSINSGG
SSGGSSGSETPGTSESATPESSGGSSGGSNIMNFFKTEITLANGEIRKRPLIETNGETGEIVWDKGRDFA
TVRKVLSMPQVNIVKKTEVQTGGFSKESIRPKRNSDKLIARKKDWDPKKYGGFVSPTVAYSVLWAKVE
KGKSKKLKSVKELLGITIMERSSFEKNPIDFLEAKGYKEVKKDLIIKLPKYSLFELENGRKRMLASARFL
QKGNELALPSKYVNFLYLASHYEKLKGSPEDNEQKQLFVEQHKHYLDEIIEQISEFSKRVILADANLDK
VLSAYNKHRDKPIREQAENIIHLFTLTNLGAPRAFKYFDTTIDRKVYRSTKEVLDATLIHQSITGLYETRID
LSQLGGDGGSGGSGGSGGSGGSGGSGGDKKYSIGLAIGTNSVGWAVITDEYKVPSKKFKVLGNTDRHS
IKKNLIGALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPAIKKGILQ
TVKWDELVKVMGRHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSDNVPSEEV
VKKMKNYWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYPKLESEFVYGDY
KVYDVRKMIAKSEQEIGKATAKYFFYSSGGSKRTADGSEEEPKKKRKV
ABE8e-NG-CP1041 monomer editor: NLS, linker, TadA*, SpCas9-NG-CPl041
(SEQ ID NO: 459)
MKRTADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSE
SATPESSGGSSGGSNIMNFFKTEITLANGEIRKRPLIETNGETGEIVWDKGRDFATVRKVLSMPQVNIVK
KTEVQTGGFSKESIRPKRNSDKLIARKKDWDPKKYGGFVSPTVAYSVLVVAKVEKGKSKKLKSVKELLG
ITIMERSSFEKNPIDFLEAKGYKEVKKDLIIKLPKYSLFELENGRKRMLASARFLQKGNELALPSKYVNF
LYLASHYEKLKGSPEDNEQKQLFVEQHKHYLDEIIEQISEFSKRVILADANLDKVLSAYNKHRDKPIREQ
AENIIHLFTLTNLGAPRAFKYFDTTIDRKVYRSTKEVLDATLIHQSITGLYETRIDLSQLGGDGGSGGSGG
SGGSGGSGGSGGDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDLAEDAKLQLSKD
TYDDDLDNLLAQIGDQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKSEETITPWNF
EEVVDKGASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKWDELVKVMGR
HKPENIVIEMARENQTTQKGQKNSRERMKRIEEGIKELGSQILKEHPVENTQLQNEKLYLYYLQNGRD
MYVDQELDINRLSDYDVDHIVPQSFLKDDSIDNKVLTRSDKNRGKSDNVPSEEWKKMKNYWRQLLNA
KLITQRKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAKSEQE
IGKATAKYFFYSSGGSKRTADGSEFEPKKKRKV
ABE8e-iSpyMac dimer editor: NLS, wtTadA, linker, TadA*, SpCas9-iSpyMac
(SEQ ID NO: 462)
MKRTADGSEFESPKKKRKVSEVEFSHEYWMRHALTLAKRAWDEREVPVGAVLVHNNRVIGE
GWNRPIGRHDPTAHAEIMALRQGGLVMQNYRLIDATLYVTLEPCVMCAGAMIHSRIGRVV
FGARDAKTGAAGSLMDVLHHPGMNHRVEITEGILADECAALLSDFFRMRRQEIKAQKKAQ
SSTDSGGSSGGSSGSEYPGYSESAYPESSGGSSGGSSEVEFSHEYWMRHALTLAKRARDEREVPVG
AVLVLNNRVIGEGWNRAIGLHDPTAHAEIMALRQGGLVMQNYRLIDATLYVTFEPCVMCAGAMIHSRIG
RVVFGVRNSKRGAAGSLMNVLNYPGMNHRVEITEGILADECAALLCDFYRMPRQVFNAQKKAQSSINS
GGSSGGSSGSETPGTSESATPESSGGSSGGSDKKYSIGLDIGTNSVGWAVITDEYKVPSKKFKVLGNTD
RHSIKKNLIGALLFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTVYNELTKVKY
VFEGMRKPAFLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKNRGKSDNVPS
EEVVKKMKNYWRQLLNAKLITQRKFDNLTKAERGGLSELDKAGFIKRQLVETRQITKHVAQILDSRMN
TKYDENDKLIREVKVITLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIETNGETGEIVWDKGRDF
ATVRKVLSMPQVNIVKKTESGGSKRTADGSEFEPKKKRKV
ABE8e-iSpyMac monomer editor: NLS, linker, TadA*, SpCas9-iSpyMac
(SEQ ID NO: 470)
MKRYADGSEFESPKKKRKVSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFGVRNSKRGAAGSL
MNVLNYPGMNHRVEITEGILADECAALLCDFYRMPRQVFNAQKKAQSSINSGGSSGGSSGSETPGTSE
SATPESSGGSSGGSDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFLSGEQK
KAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDHIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLSMPQVNIVKKT
ESGGSKRTADGSEFEPKKKRKV

### Reduced Off-Target Effects

### Reduced RNA Editing Effects

The adenine base editors disclosed herein have reduced and/or low RNA editing effects. In some embodiments, the base editors are evolved or engineered to have reduced RNA editing effects. The term “RNA editing effects,” as used herein, refers to the introduction of modifications (e.g. deaminations) of nucleotides within cellular RNA, e.g., messenger RNA (mRNA). An important goal of DNA base editing efficiency is the modification (e.g. deamination) of a specific nucleotide within DNA, without introducing modifications of similar nucleotides within RNA. RNA editing effects are “low” or “reduced” when a detected mutation is introduced into RNA molecules at a frequency of 0.3% or less.

The present disclosure further provides methods of administering the disclosed adenine base editors wherein the method yields reduced and/or low RNA editing effects. The present disclosure further provides adenine base editors that induce (or yield, provide or cause) reduced and/or low RNA editing effects. In some embodiments, the base editors provide an average adenosine (A) to inosine (I) (A-to-I) editing frequency in cellular mRNA transcripts of 0.3% or less. In some embodiments, the base editors provide an average adenosine (A) to inosine (I) (A-to-I) actual and/or consistent editing frequencies in RNA of about 0.3% or less. The base editors may provide actual or average A-to-I editing frequencies in RNA of about 0.5% or less, 0.4% or less, 0.35% or less, 0.25% or less, 0.2% or less, 0.15% or less, 0.12% or less, 0.1% or less, 0.08% or less, or 0.075% or less. In particular embodiments, the base editors provide an average A-to-I editing frequency of about 0.25%. In particular embodiments, the base editor ABE-8e(V106W) (SEQ ID NO: 188) induces an average A-to-I editing frequency of 0.25%.

In some embodiments, the methods induce (or provide or cause) an average adenine (A) to inosine (I) (A-to-I) editing frequency across the mRNA transcriptome of a human cell (e.g. an HEK293 cell) of about 0.3% or less. The methods may induce actual or average A-to-I transcriptome-wide editing frequencies in RNA of about 0.5% or less, 0.4% or less, 0.35% or less, 0.25% or less, 0.2% or less, 0.15% or less, 0.12% or less, 0.1% or less, 0.08% or less, or 0.075% or less. In particular embodiments, the methods induce a human mRNA transcriptome-wide average A-to-I editing frequency of 0.25%.

### Reduced Off-Target DNA Editing Effects

Guide RNA-dependent off-target base editing has been reduced through strategies including installation of mutations that increase DNA specificity into the Cas9 component of base editors, adding 5′ guanosine nucleotides to the sgRNA, or delivery of the base editor as a ribonucleoprotein complex (RNP). Guide RNA-independent off-target editing can arise from binding of the deaminase domain of a base editor to C or A bases in a Cas9-independent manner. Recent studies characterized guide RNA-independent off-target DNA editing activity of BE3, the original cytosine base editor, in mouse embryos and in rice. In contrast with BE3, ABE7.10 in these studies did not result in detectable guide RNA-independent off-target DNA mutations. However, the Examples below establish that ABE7.10 and some evolved ABE7.10 variants such as ABE-8e do in fact exhibit detectable guide RNA-independent off-target DNA mutations. However, some evolved ABE7.10 variants provided herein, such as ABE-8e(V106W), exhibit reduced Cas9-independent off-target DNA mutations relative to ABE7.10. The off-target effects of the disclosed adenine base editors may be measured using assays and methods disclosed in and International Application No. PCT/US2020/062428, filed Nov. 25, 2020, incorporated herein by reference.

In some aspects, provided herein are adenine base editors and methods of editing DNA by contacting DNA with any of these disclosed base editors that generate (or cause) reduced off-target effects. In various embodiments, methods are designed for determining the off-target editing frequencies of napDNAbp domain-independent (e.g., Cas9-independent) (or gRNA-independent) off-target editing events. Editing events may comprise deamination events of an ABE. Off-target deamination events that are dependent on the napDNAbp-guide RNA complex tend to be in sequences that have high sequence identity (e.g., greater than 60% sequence identity) to the target sequence. These types of events arise because of imperfect hybridization of the napDNAbp-guide RNA complex to sequences that share identity with the target sequence. In contrast, off-target events that occur independently of the napDNAbp-guide RNA complex arise as a result of stochastic binding of the base editor to DNA sequences (often sequences that do not share high sequence identity with the target sequence) due to an intrinsic affinity of the base editor of the nucleotide modification domain (e.g., the deaminase domain) of the base editor with DNA. NapDNAbp-independent (e.g., Cas9-independent) editing events arise in particular when the base editor is overexpressed in the system under evaluation, such as a cell or a subject.

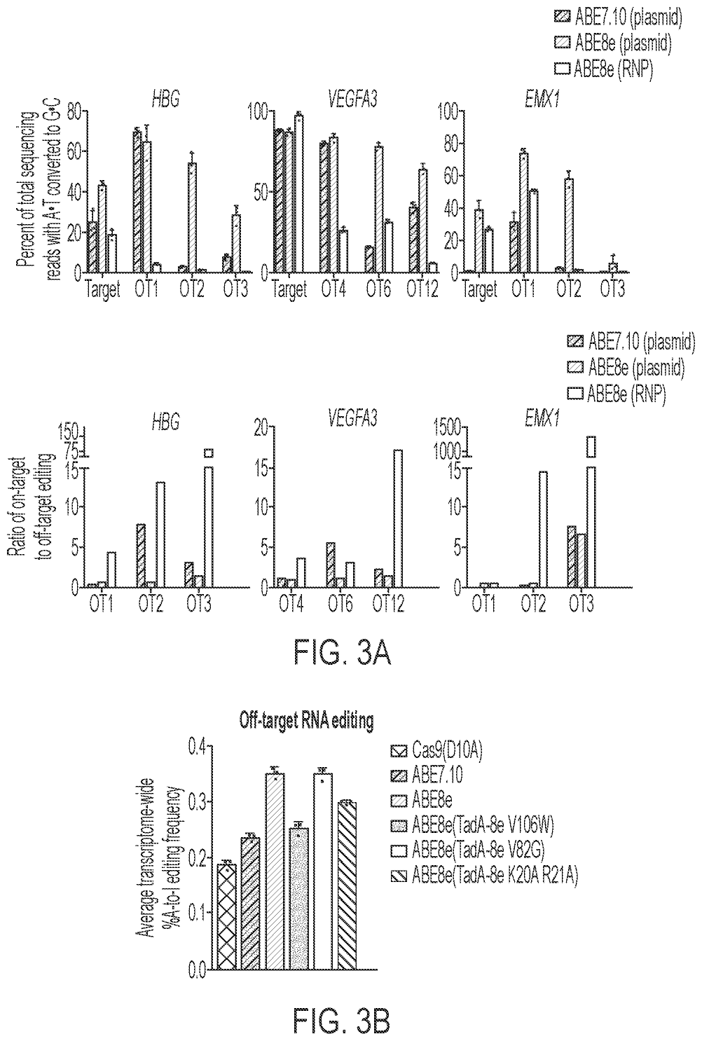

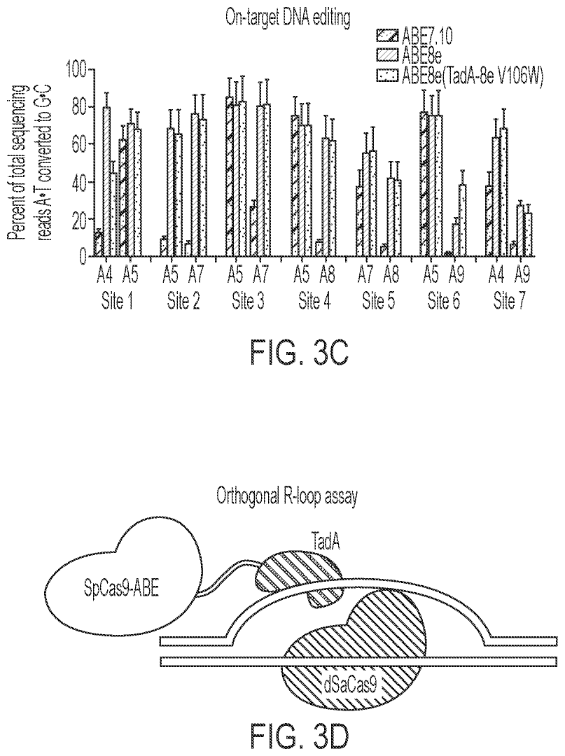

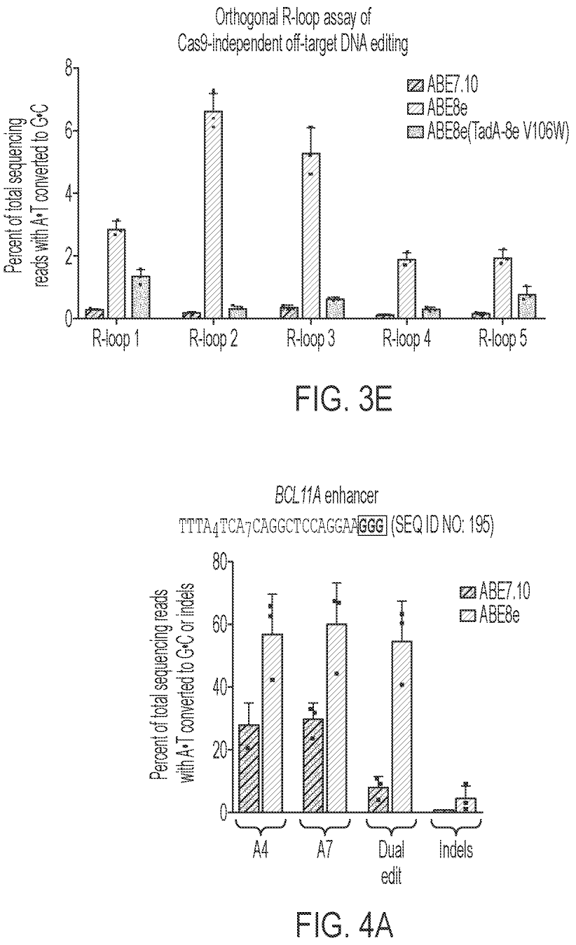

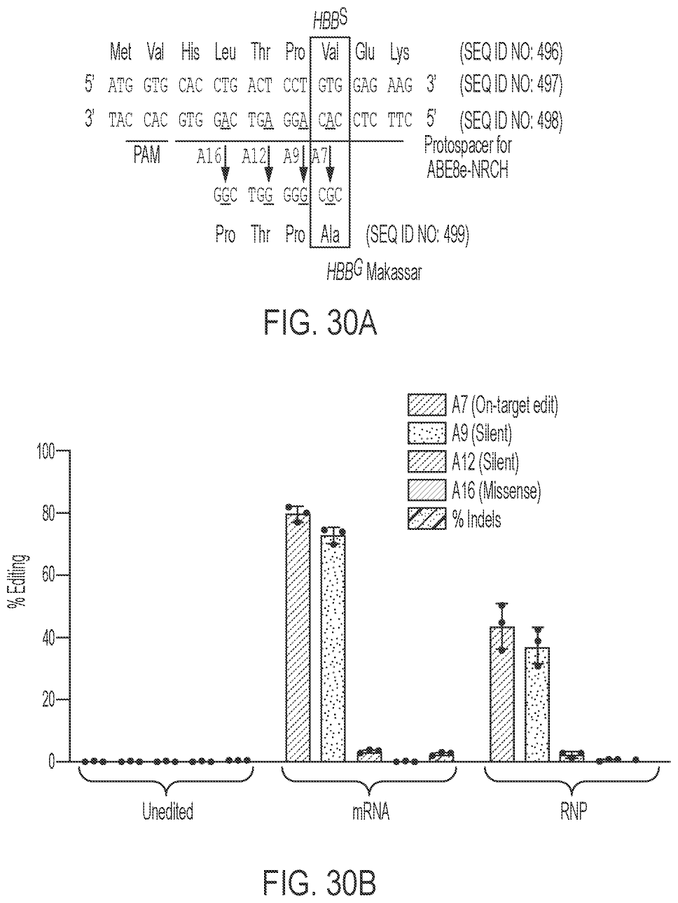

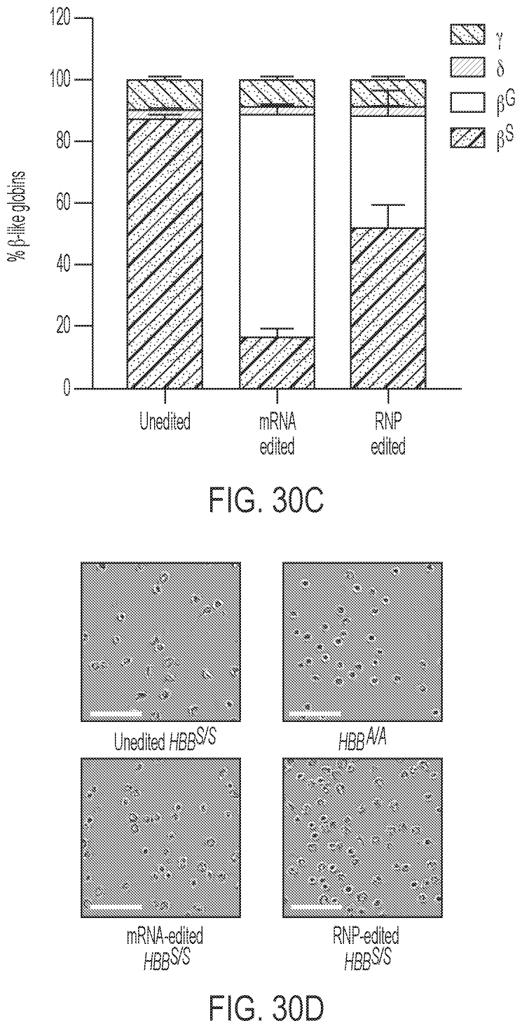

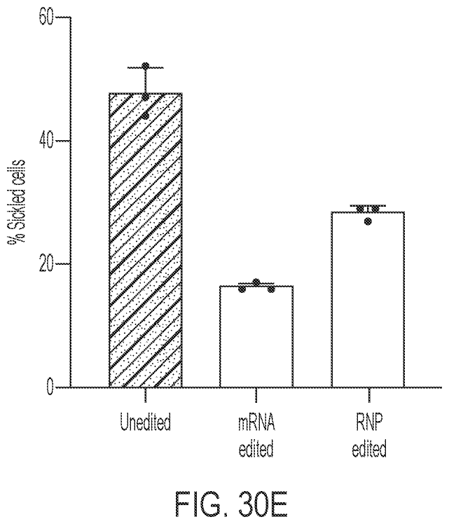

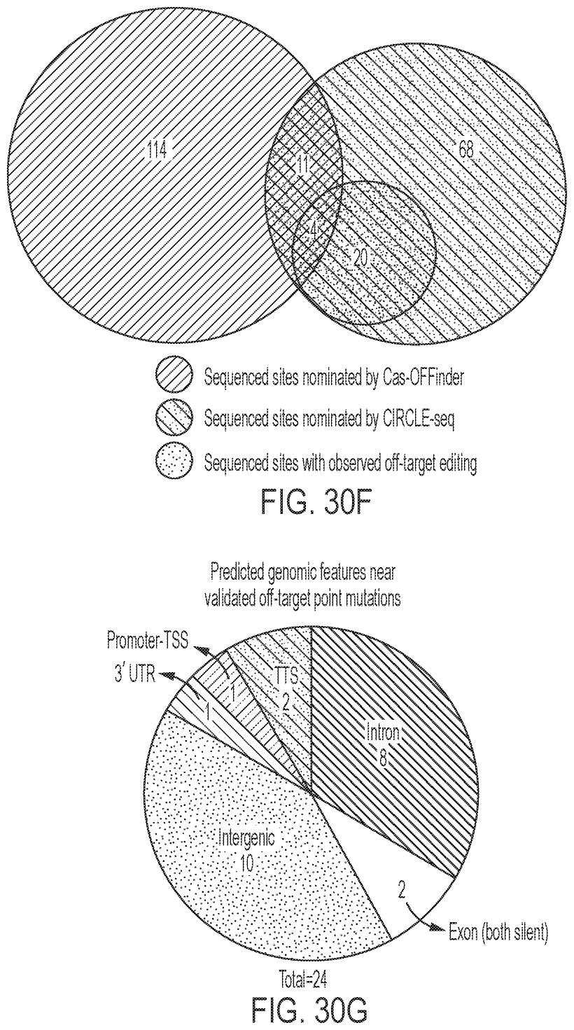

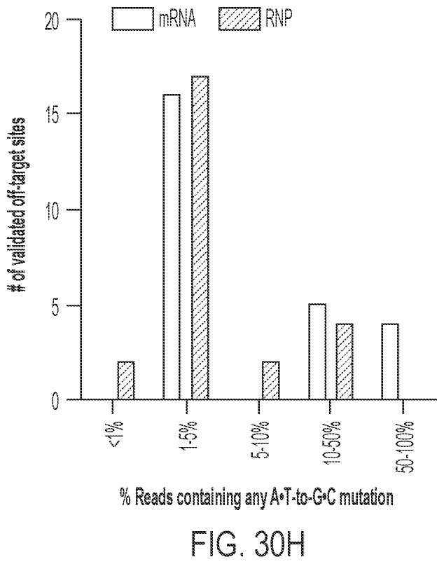

The disclosed ABEs exhibit low off-target editing frequencies, and in particular low Cas9-independent off-target editing frequencies, while exhibiting high on-target editing efficiencies. For example, the ABE8e (V106W) base editor may exhibit off-target editing frequencies of 0.32% or less while maintaining on-target editing efficiencies of about 80% or more, in target sequences in mammalian cells. (See FIG. 3E.) As another example, the ABE8e base editor may exhibit off-target editing frequencies of 1.9% or less while maintaining on-target editing efficiencies of about 80% or more, in target sequences in mammalian cells. As another example, the ABE8e-NRCH (monomer) base editor may exhibit indel frequencies of 2.8% or less and/or average off-target editing frequencies of 5% or less, while maintaining on-target editing efficiencies of 80% in target sequences in human cells. (See FIGS. 30B, 30H.) The disclosed ABEs exhibit low to no clinically relevant off-target effects (e.g., point mutations in clinically relevant exons).

In some embodiments, the disclosed adenosine deaminases exhibit low off-target editing frequencies, and in particular low Cas9-independent off-target editing frequencies, while exhibiting high on-target editing efficiencies when used a variety of Cas homologs and other napDNAbps. In some embodiments, the TadA-8e deaminase or TadA-8e(V106W) deaminase may exhibit off-target editing frequencies of 0.32% or less while maintaining on-target editing efficiencies of about 80% or more, in target sequences in mammalian cells, when used with a variety of napDNAbps, such as SpCas9, SaCas9, CP1041, and SaKKH-Cas9.

In some embodiments, the disclosed base editors cause off-target DNA editing (e.g. off-target deamination) frequencies of less than 1.5% (such as less than 1.25%, less than 1.0%, less than 0.75%, or less than 0.5%). The disclosed base editors may further provide (or yield) on-target editing efficiencies of greater than 60% (such as greater than 70%, greater than 75%, greater than 80%, or greater than 85%) at the target nucleobase pair for one or more base editors under evaluation. These methods may yield an on-target editing efficiency of greater than 50% and a frequency of off-target editing of less than 1.5% for one or more base editors under consideration.

In some embodiments, the disclosed ABEs and editing methods comprising the step of contacting a DNA with any of the disclosed ABEs result in an actual or average off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less. In some embodiments, the disclosed editing methods result in an actual or average off-target DNA editing frequency of 0.5%, less than 0.5%, less than 0.4%, less than 0.35%, less than 0.3%, less than 0.25%, less than 0.2%, or less than 0.1%. In a particular embodiment, the methods result in an actual or average off-target DNA editing frequency of about 0.32% to about 1.3% (for instance, methods for evaluating the off-target frequencies of ABEs comprising TadA8e-V106W deaminase). These off-target editing frequencies may be obtained in sequences having any level of sequence identity to the target sequence. As used herein to refer to off-target DNA editing frequencies, the modifier “average” refers to a mean value over all editing events detected at sites other than a given target nucleobase pair (e.g., as detected by high-throughput sequencing).

In some embodiments, the disclosed editing methods further result in an actual or average Cas9-independent off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less. In other words, the disclosed editing methods further result in an actual or average off-target DNA editing frequency of about 2.0% or less, 1.75% or less, 1.5% or less, 1.2% or less, 1% or less, 0.9% or less, 0.8% or less, 0.75% or less, 0.7% or less, 0.65% or less, or 0.6% or less in sequences having 60% or less sequence identity to the target sequence. In some embodiments, the disclosed editing methods result in an actual or average off-target DNA editing frequency 0.5%, less than 0.5%, less than 0.4%, less than 0.35%, less than 0.3%, less than 0.25%, less than 0.2%, or less than 0.1%, in sequences having 60% or less sequence identity to the target sequence. In some embodiments, these editing frequencies are obtained in sequences comprising protospacer sequences having 5, 6, 7, 8, 9, 10, or more than 10 mismatches relative to protospacer sequence of the target sequence. In a particular embodiment, the methods result in an actual or average Cas9-independent off-target DNA editing frequency of 0.4% or less.

In various embodiments, the disclosed editing methods result in an on-target DNA base editing efficiency of at least about 35%, 40%, 50%, 60%, 70%, 80%, 85%, 90%, 95%, 98%, or 99% at the target nucleobase pair. The step of contacting may result in in a DNA base editing efficiency of at least about 60%, 61%, 62%, 63%, 64%, 65%, 66%, 67%, 68%, 69%, 70%, 71%, 72%, 73%, 74%, or 75%. In particular, the step of contacting results in on-target base editing efficiencies of greater than 75%. In certain embodiments, base editing efficiencies of 99% may be realized.

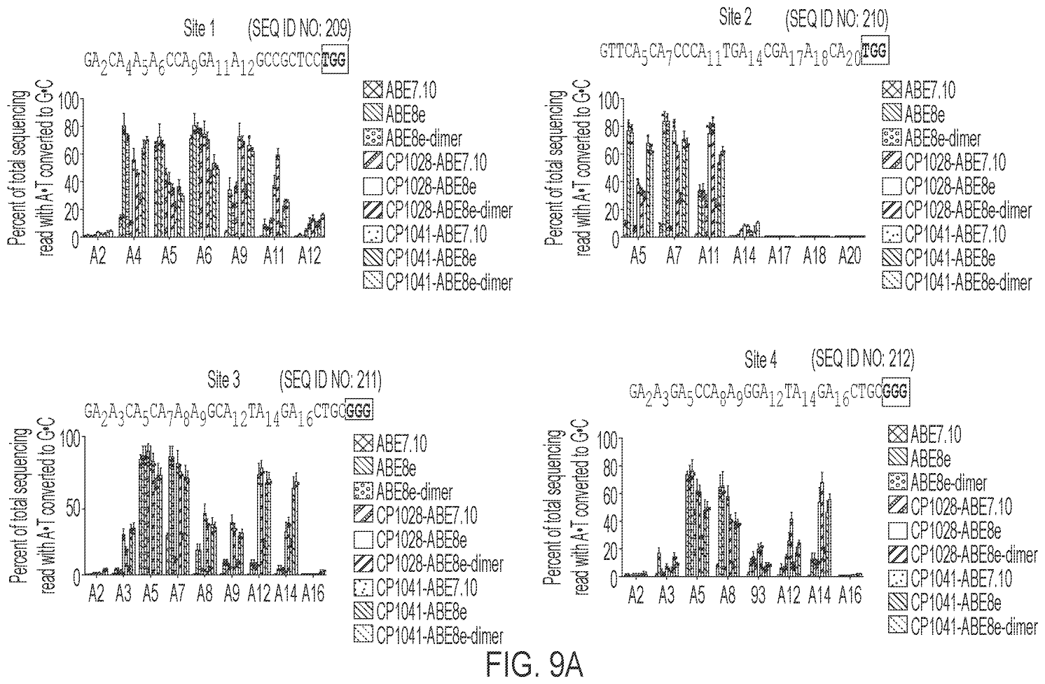

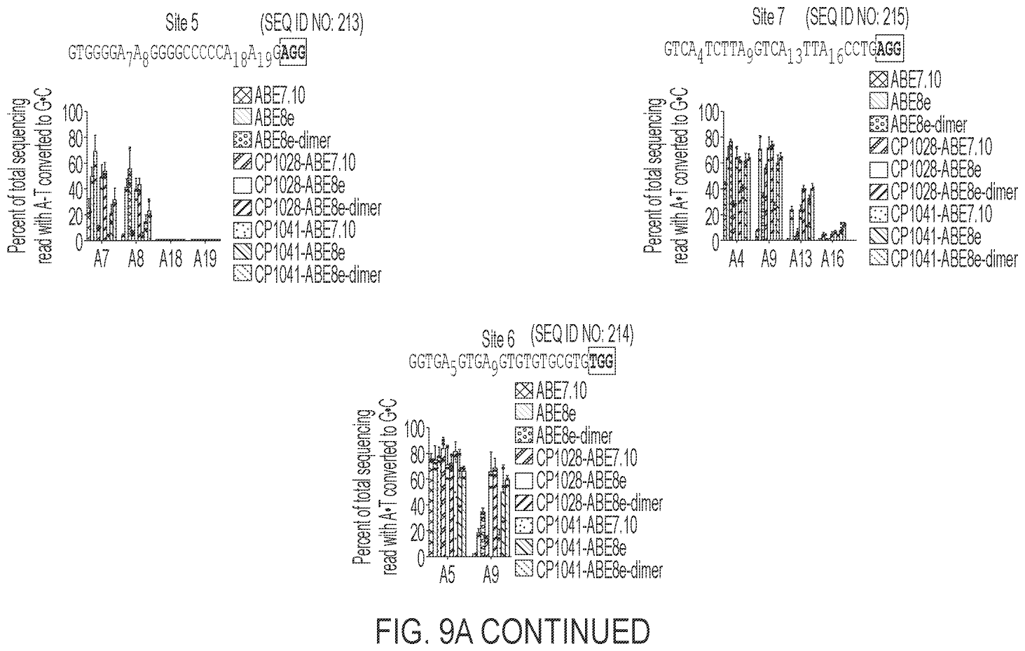

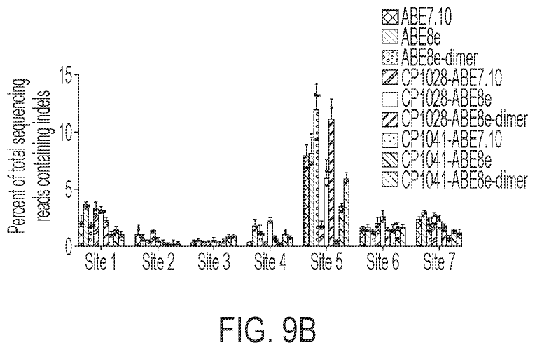

The adenosine deaminase variants of the disclosure (such as TadA8e) exhibit higher editing efficiencies when paired in a base editor with certain Cas9 variants than existing adenosine deaminases (e.g., TadA7.10). In some embodiments, a base editor containing any of the disclosed adenosine deaminases and a CP1028-SpCas9 napDNAbp domain exhibits at least 75% on-target editing efficiencies. In some embodiments, a base editor containing any of the disclosed adenosine deaminases and a CP1041-SpCas9 napDNAbp domain exhibits at least 60% on-target editing efficiencies. These editing efficiencies are significantly greater than corresponding efficiencies of the CP1041-ABE7.10 and CP1028-ABE7.10 adenine base editors. (See FIG. 9A.) In some embodiments, a base editor containing any of the disclosed adenosine deaminases and a Cas9-NRCH napDNAbp domain exhibits at least 60% on-target editing efficiencies at an adenine base in a clinically relevant genomic target such as an HBB gene, or an HBG promoter. This editing efficiency is significantly greater than corresponding efficiency of the ABE7.10-NRCH adenine base editor. (See FIG. 34.)

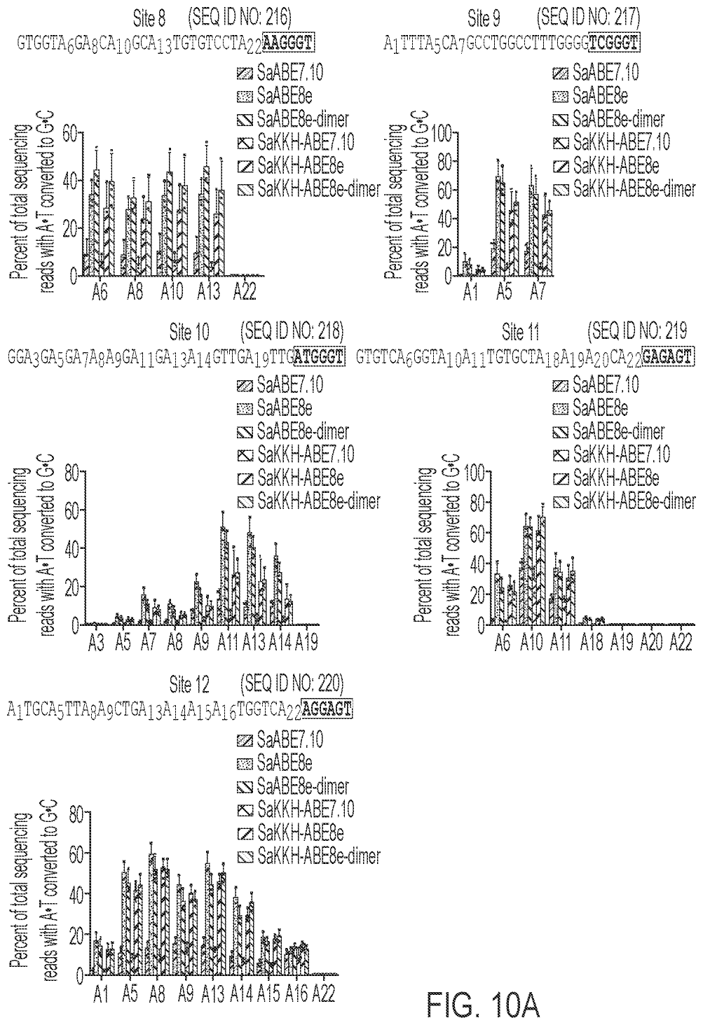

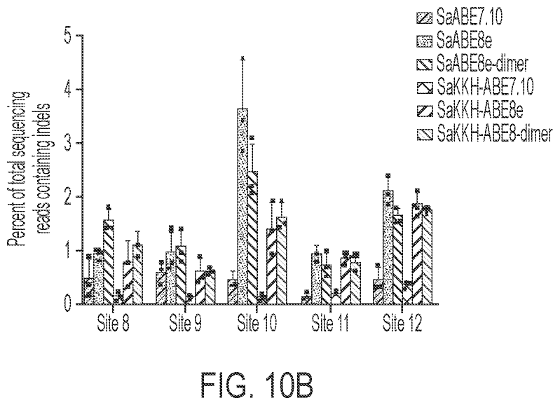

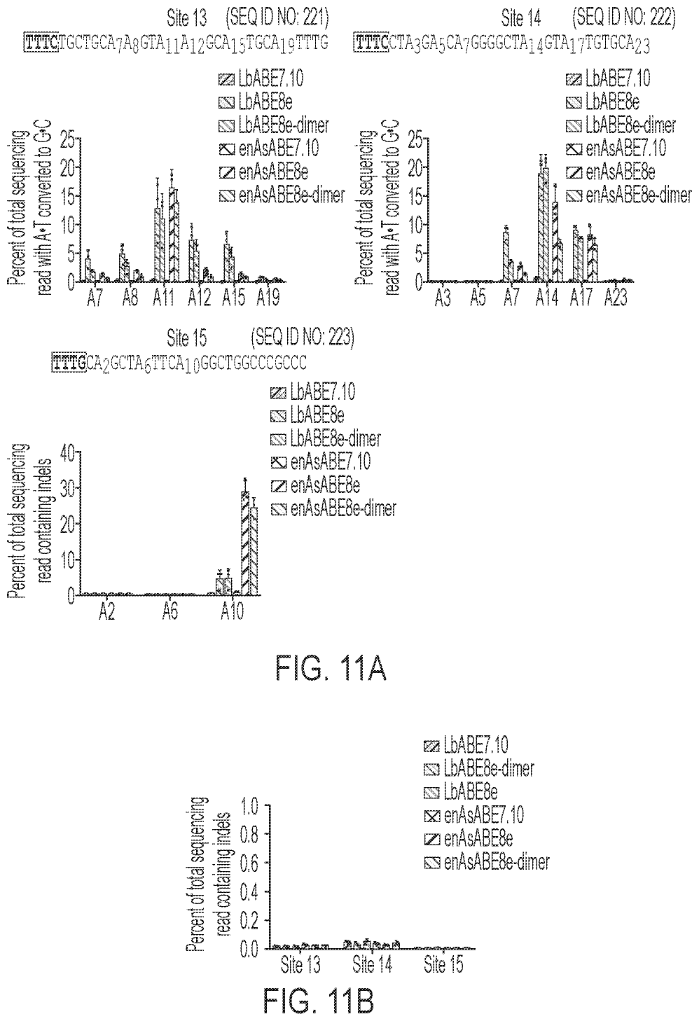

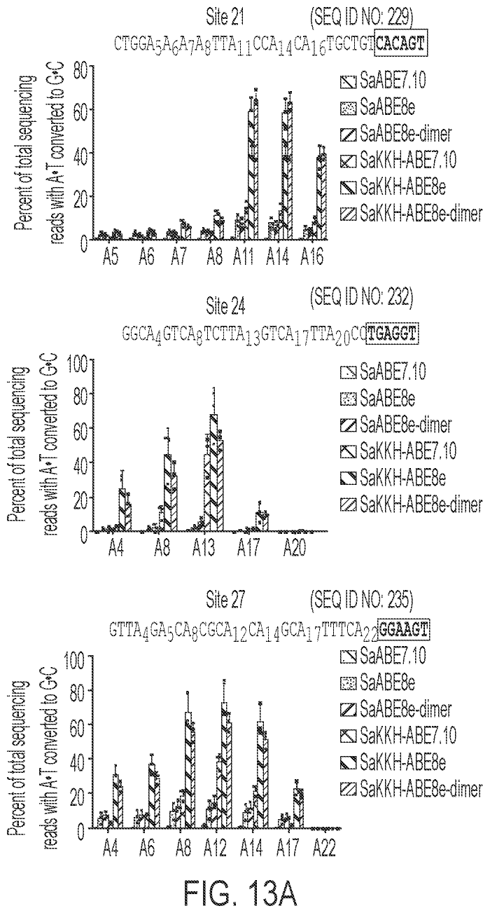

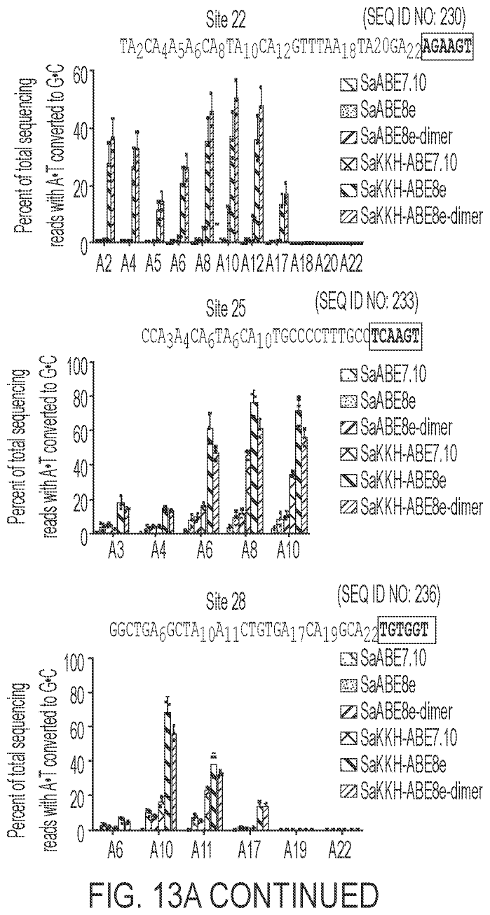

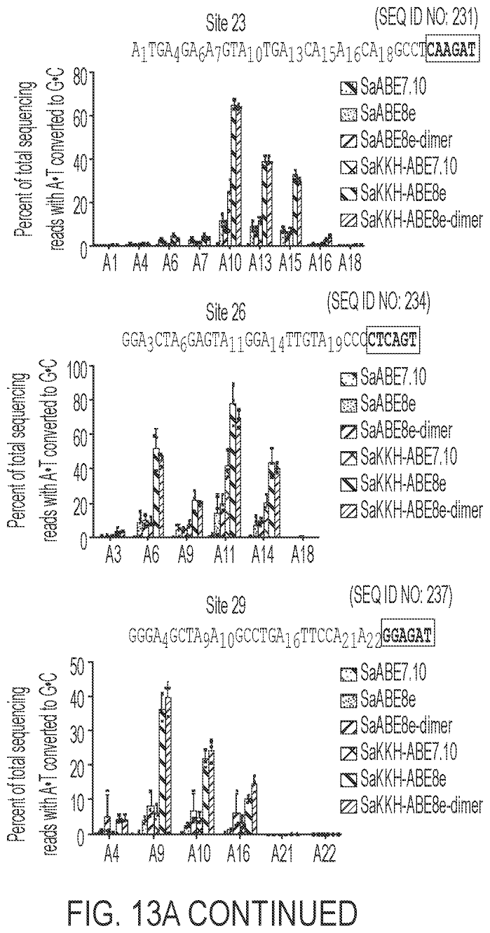

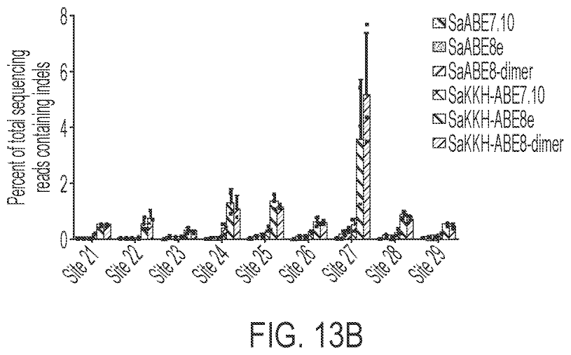

Further, the adenosine deaminase variants of the disclosure exhibit higher editing efficiencies when paired in a base editor with Cas9 homologs derived from S. aureus than existing adenosine deaminases (e.g., TadA7.10). In some embodiments, a base editor containing any of the disclosed adenosine deaminases and an SaCas9n napDNAbp domain or an SaCas9-KKH napDNAbp domain exhibits at least 38%, or at least 45%, on-target editing efficiencies. These editing efficiencies are significantly greater than corresponding efficiencies of the SaABE7.10 and SaKKH-ABE7.10 adenine base editors. (See FIGS. 10A and 13A.)

Further, the adenosine deaminase variants of the disclosure exhibit higher editing efficiencies when paired in a base editor with AsCas12a and LbCas12a homologs than existing adenosine deaminases (e.g., TadA7.10). Base editors containing a fusion of TadA7.10 to engineered AsCas12a (enAsCas12a) and LbCas12a exhibit little to no activity on adenine targets. However, in some embodiments, a base editor containing any of the disclosed adenosine deaminases and an enAsCas12a napDNAbp domain exhibits about 15% or greater adenine deamination activity. In some embodiments, a base editor containing any of the disclosed adenosine deaminases and an LbCas12a napDNAbp domain exhibits at least about 12%, 15%, or 18% adenine deamination activity. (See FIG. 11A.)

In various embodiments, the disclosed editing methods result in a ratio of on-target:off-target editing of about 25:1, 50:1, 65:1, 75:1, 80:1, 85:1, 90:1, 95:1, 100:1, 110:1, 125:1, or more than 125:1. In various embodiments, the disclosed editing methods result in a ratio of on-target:off-target editing of about 150:1, 200:1, 300:1, 400:1, 500:1, 600:1, 700:1, 800:1, 900:1, 1000:1, 1100:1, 1200:1, 1250:1, 1275:1, 1300:1, 1325:1, 1350:1, 1400:1, 1500:1, or more than 1500:1. In particular embodiments, the disclosed editing methods result in a ratio of on-target:off-target editing of about 90:1 or more in an HBG gene, such as a human HBG gene. In particular embodiments, the disclosed editing methods result in a ratio of on-target:off-target editing of about 1300:1 or more in an EMX1 gene, such as a human EMX1 gene. As used herein, a ratio of on-target:off-target editing is equivalent to a ratio of sequencing reads reflecting on-target deaminations relative to deaminations of known or predicted off-target sites, or candidate off-target sites. Candidate off-target sites may be identified, and hence the ratio of on-target:off-target editing may be measured, using an experimental assay or a computation algorithm (e.g., Cas-OFFinder). For example, candidate off-target sites may be identified using an experimental assay such as EndoV-Seq, GUIDE-Seq, or CIRCLE-Seq. In some embodiments, the ratios of on-target editing:off-target editing relies on the use of EndoV-Seq.

In some embodiments, the disclosed editing methods result in a ratio of on-target:off-target editing that is equivalent to the ratio of intended point mutations:unintended point mutations. In some embodiments, the disclosed editing methods result in a ratio of intended point mutations to unintended point mutations that is at least 25:1, at least 30:1, at least 40:1, at least 50:1, at least 75:1, at least 90:1, at least 100:1, at least 150:1, at least 200:1, at least 250:1, at least 500:1, at least 1000:1, at least 1100:1, at least 1200:1, at least 1250:1, at least 1300:1, at least 1350:1, at least 1400:1, at least 1500:1, or more.

In some embodiments, the disclosed editing methods result in, and the disclosed base editors generate, a very low degree of bystander edits (i.e., synonymous off-target point mutations at nucleobases that are near the target base and do not change the outcome of the intended editing method). In some embodiments, the disclosed editing methods result in less than 10, less than 9, less than 8, less than 7, less than 6, less than 5, less than 4, less than 3, less than 2, less than 1, or zero non-silent bystander edits. For example, editing methods using the disclosed ABE8e-NRCH editor result in zero non-silent bystander edits.

### Reduced Indel Frequencies

Some aspects of the disclosure are based on the recognition that any of the adenine base editors provided herein are capable of modifying a specific DNA base without generating a significant proportion of indels. An “indel”, as used herein, refers to the insertion or deletion of a nucleotide base within a DNA substrate. Such insertions or deletions can lead to frame shift mutations within a coding region of a gene. In some embodiments, it is desirable to generate adenine base editors that efficiently modify (e.g. mutate or deaminate) a specific nucleotide within a DNA, without generating a large number of insertions or deletions (i.e., indels) in the nucleic acid (while at the same time having lower RNA editing effects than existing adenine base editors).

In certain embodiments, any of the adenine base editors provided herein are capable of generating a greater proportion of intended modifications (e.g., point mutations or deaminations) versus indels. In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method, for example the methods used in the below Examples. In some embodiments, indel frequencies correspond to the percent of total sequencing reads at a target sequence that contain indels. Accordingly, to calculate indel frequencies, sequencing reads are scanned for exact matches to two 10-bp sequences that flank both sides of a window in which indels might occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively.

In some embodiments, the adenine base editors provided herein are capable of limiting formation of indels in a region of a DNA substrate. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein may induce an indel formation at a region of a nucleic acid at frequencies of less than 1%, less than 1.5%, less than 2%, less than 2.5%, less than 2.8%, less than 3%, less than 3.5%, less than 4%, less than 4.5%, less than 5%, less than 6%, less than 7%, less than 8%, less than 9%, less than 10%, less than 12%, less than 15%, or less than 20%. In some embodiments, any of the base editors provided herein may induce or generate less than 20%, 19%, 18%, 16%, 14%, 12%, 10%, 8%, 6%, 4%, 3%, 2%, 1%, 0.5%, 0.2%, 0.1%, or 0.05% indel formation when contacted with a nucleic acid comprising a target sequence. The number of indels formed at a nucleic acid region may depend on the amount of time a nucleic acid (e.g., a nucleic acid within the genome of a cell) is exposed to a base editor. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to an adenine base editor.

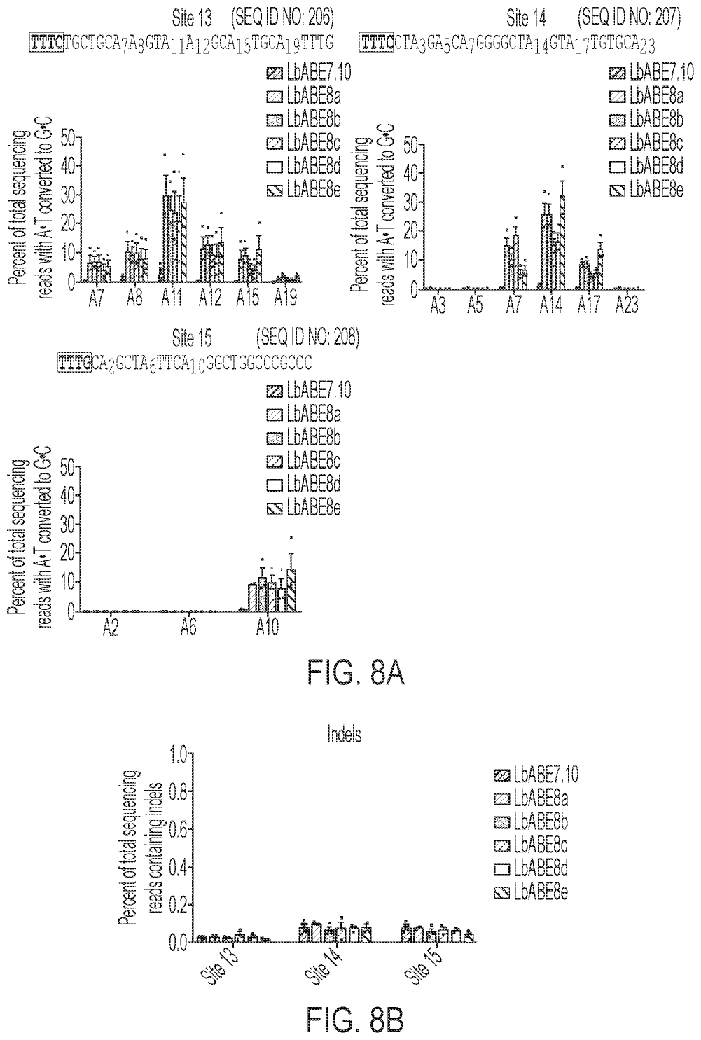

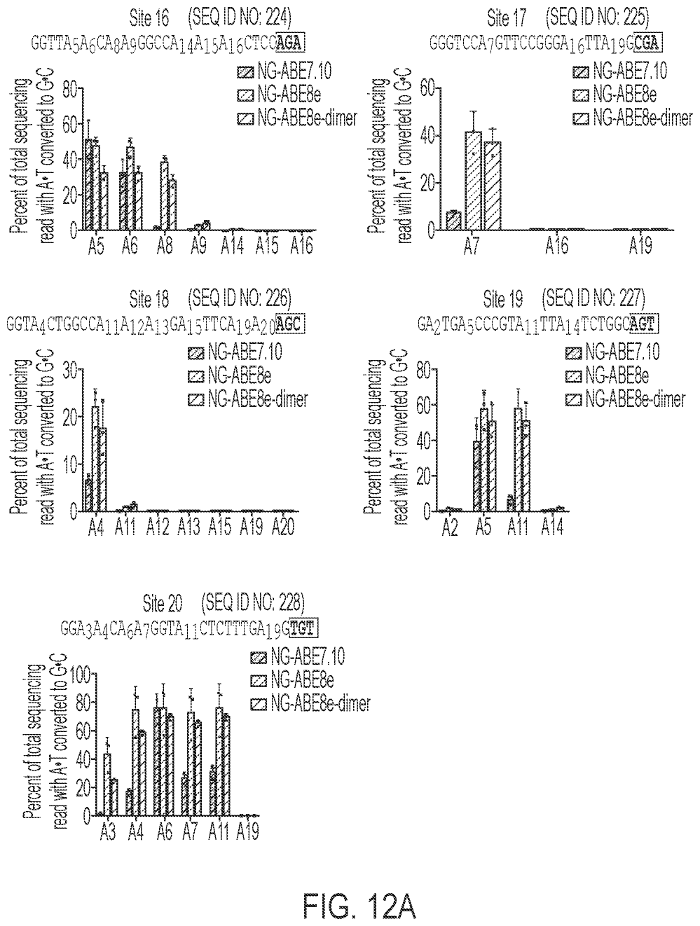

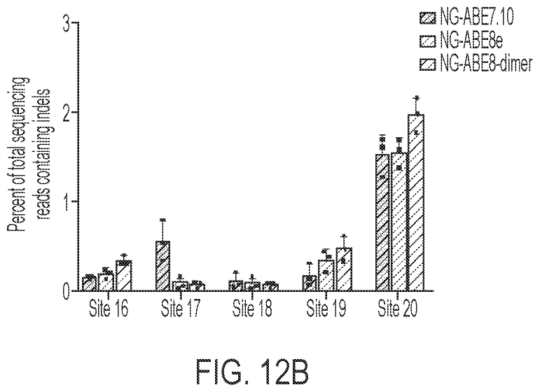

In some embodiments, the LbABE8e base editor may induce an indel formation at a region of a nucleic acid comprising a target sequence at frequencies of less than 0.2% (see FIGS. 8B and 11B). In some embodiments, the CP1028-ABE8e and CP1041-ABE8e base editors may induce indel formation at a region of a nucleic acid comprising a target sequence at frequencies of less than 0.5% (see FIG. 9B). In some embodiments, the CP1028-ABE8e-dimer and CP1041-ABE8e-dimer base editors may induce indel formation at a region of a nucleic acid comprising a target sequence at frequencies of less than 0.5%. In some embodiments, the SaABE8e and SaKKH-ABE8e base editors may induce indel formation at a region of a nucleic acid comprising a target sequence at frequencies of less than 1.25% (see FIGS. 10B and 13B). In some embodiments, the NG-ABE8e base editor may induce indel formation at a target sequence at frequencies of less than 0.5 or less than 0.25% (see FIG. 12B).

In some embodiments, the disclosed editing methods that use the disclosed ABEs may result in less than 20%, 19%, 18%, 16%, 14%, 12%, 10%, 8%, 6%, 4%, 2%, 1.5%, 1%, 0.5%, 0.2%, or 0.1% indel formation in a a nucleic acid (e.g., a DNA) comprising a target sequence.

Some aspects of the disclosure are based on the recognition that any of the base editors provided herein are capable of efficiently generating an intended mutation, such as a point mutation, in DNA (e.g. DNA within a genome of a subject) without generating a significant number of unintended mutations, such as unintended point mutations. In some embodiments, a intended mutation is a mutation that is generated by a specific base editor bound to a gRNA, specifically designed to generate the intended mutation (e.g. deamination). In some embodiments, the intended mutation is a mutation associated with a disease or disorder, such as sickle cell disease. In some embodiments, the intended mutation is an adenine (A) to guanine (G) point mutation associated with a disease or disorder. In some embodiments, the intended mutation is a thymine (T) to cytosine (C) point mutation associated with a disease or disorder. In some embodiments, the intended mutation is an adenine (A) to guanine (G) point mutation within the coding region of a gene. In some embodiments, the intended mutation is a thymine (T) to cytosine (C) point mutation within the coding region of a gene.

In some embodiments, the intended mutation is a deamination that generates a stop codon, for example, a premature stop codon within the coding region of a gene. In some embodiments, the intended mutation is a mutation that eliminates a stop codon. In some embodiments, the intended mutation eliminates a stop codon comprising the nucleic acid sequence 5′-TAG-3′, 5′-TAA-3′, or 5′-TGA-3′.

In some embodiments, the intended mutation is a deamination that alters the regulatory sequence of a gene (e.g., a gene promoter or gene repressor). In some embodiments, the intended mutation is a deamination introduced into the gene promoter. In particular embodiments, the deamination introduced into the gene promoter leads to a decrease in the transcription of a gene operably linked to the gene promoter. In other embodiments, the deamination leads to an increase in the transcription of a gene operably linked to the gene promoter.

In some embodiments, the intended mutation is a deamination that alters the splicing of a gene. Accordingly, in some embodiments, the intended deamination results in the introduction of a splice site in a gene. In other embodiments, the intended deamination results in the removal of a splice site.

In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described in this section and the following section of the disclosure may be applied to any of the base editors, or methods of using the base editors provided herein.

### Guide Sequences (e.g., Guide RNAs)

The present disclosure further provides guide RNAs for use in accordance with the disclosed methods of editing. The disclosure provides guide RNAs that are designed to recognize target sequences. Such gRNAs may be designed to have guide sequences (or “spacers”) having complementarity to a protospacer within the target sequence.

Guide RNAs are also provided for use with one or more of the disclosed adenine base editors, e.g., in the disclosed methods of editing a nucleic acid molecule. Such gRNAs may be designed to have guide sequences having complementarity to a protospacer within a target sequence to be edited, and to have backbone sequences that interact specifically with the napDNAbp domains of any of the disclosed base editors, such as Cas9 nickase domains of the disclosed base editors.

In various embodiments, the base editors may be complexed, bound, or otherwise associated with (e.g., via any type of covalent or non-covalent bond) one or more guide sequences. The guide sequence becomes associated or bound to the base editor and directs its localization to a specific target sequence having complementarity to the guide sequence or a portion thereof. The particular design embodiments of a guide sequence will depend upon the nucleotide sequence of a genomic target sequence (i.e., the desired site to be edited) and the type of napDNAbp (e.g., type of Cas9 protein) present in the base editor, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of the napDNAbp (e.g., a Cas9 or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, each gRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that is complementary to a target sequence (or off-target site).

In some embodiments, a guide sequence is less than about 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence, such as by transfection with vectors encoding the components of a base editor disclosed herein, followed by an assessment of preferential cleavage within the target sequence. Similarly, cleavage of a target polynucleotide sequence may be evaluated in situ by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker & Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106(1): 23-24; and PA Carr & GM Church, 2009, Nature Biotechnology 27(12): 1151-62). Additional algorithms may be found in Chuai, G. et al., DeepCRISPR: optimized CRISPR guide RNA design by deep learning, Genome Biol. 19:80 (2018), and U.S. Application Ser. No. 61/836,080 and U.S. Pat. No. 8,871,445, issued Oct. 28, 2014, the entireties of each of which are incorporated herein by reference.

The guide sequence of the gRNA is linked to a tracr mate (also known as a “backbone”) sequence which in turn hybridizes to a tracr sequence. A tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In certain embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides.

Non-limiting examples of single (DNA) polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(1) NNNNNNNNgtttttgtactctcaagatttaGAAAtaaatcttgcagaagctacaaagataaggctt catgccgaaatcaacaccctgtcattttatggcagggtgttttcgttatttaaTTTTTT (SEQ ID NO: 333);
(2) NNNNNNNNNNNNNNNNNNgtttttgtactctcaGAAAtgcagaagctacaaagataaggcttcatgccgaaatca acaccctgtcattttatggcagggtgttttcgttatttaaTTTTTT (SEQ ID NO: 334);
(3) NNNNNNNNNNNNNNNNNNNNgtttttgtactctcaGAAAtgcagaagctacaaagataaggcttcatgccgaa atcaacaccctgtcattttatggcagggtgtTTTTT (SEQ ID NO: 335);
(4) NNNNNNNNNNNNNNNNNNNNgttttagagctaGAAAtagcaagttaaaataaggctagtccgttatcaacttg aaaaagtggcaccgagtcggtgcTTTTTT (SEQ ID NO: 336);
(5) NNNNNNNNNNNNNNNNNNNgttttagagctaGAAATAGcaagttaaaataaggctagtccgttatcaacttga aaaagtgTTTTTTT (SEQ ID NO: 337); and
(6) NNNNNNNNNNNNNNNNNNNNgttttagagctagAAATAGcaagttaaaataaggctagtccgttatcaTT TTTTTT (SEQ ID NO: 338). In some embodiments, sequences (1) to (3) are used in combination with Cas9 from S. Thermophiles CRISPR1. In some embodiments, sequences (4) to (6) are used in combination with Cas9 from S. pyogenes. In some embodiments, the tracr sequence is a separate transcript from a transcript comprising the tracr mate sequence.

In some embodiments, the guide RNAs for use in accordance with the disclosed methods of editing comprise synthetic single guide RNAs (sgRNAs) containing modified ribonucleotides. In some embodiments, the guide RNAs contain modifications such as 2′-O-methylated nucleotides and phosphorothioate linkages. In some embodiments, the guide RNAs contain 2′-O-methyl modifications in the first three and last three nucleotides, and phosphorothioate bonds between the first three and last three nucleotides. Exemplary modified synthetic sgRNAs are disclosed in Hendel A. et al., Nat. Biotechnol. 33, 985-989 (2015), herein incorporated by reference.

In some embodiments, the guide RNAs for use in accordance with the disclosed methods of editing comprise a backbone structure that is recognized by an S. pyogenes Cas9 protein or domain, such as an SpCas9 domain of the disclosed base editors. The backbone structure recognized by an SpCas9 protein may comprise the sequence 5′-[guide sequence]-guuuuagagcuagaaauagcaaguuaaaauaaggcuaguccguuaucaacuugaaaaaguggcaccgagucggugcuuuuu-3′ (SEQ ID NO: 339), wherein the guide sequence comprises a sequence that is complementary to the protospacer of the target sequence. See U.S. Publication No. 2015/0166981, published Jun. 18, 2015, the disclosure of which is incorporated by reference herein. The guide sequence is typically 20 nucleotides long.

In other embodiments, the guide RNAs for use in accordance with the disclosed methods of editing comprise a backbone structure that is recognized by an S. aureus Cas9 protein. The backbone structure recognized by an SaCas9 protein may comprise the sequence 5′-[guide sequence]-guuuuaguacucuguaaugaaaauuacagaaucuacuaaaacaaggcaaaaugccguguuuaucucgucaacuuguuggcga gauuuuuuu-3′ (SEQ ID NO: 78).

In other embodiments, the guide RNAs for use in accordance with the disclosed methods of editing comprise a backbone structure that is recognized by an Lachnospiraceae bacterium Cas12a protein. The backbone structure recognized by an LbCas12a protein may comprise the sequence 5′-[guide sequence]-uaauuucuacuaaguguagau-3′ (SEQ ID NO: 445).

In other embodiments, the guide RNAs for use in accordance with the disclosed methods of editing comprise a backbone structure that is recognized by an Acidaminococcus sp. BV3L6 Cas12a protein. The backbone structure recognized by an AsCas12a protein may comprise the sequence 5′-[guide sequence]-uaauuucuacucuuguagau-3′ (SEQ ID NO: 446).

The sequences of suitable guide RNAs for targeting the disclosed ABEs to specific genomic target sites will be apparent to those of skill in the art based on the present disclosure. Such suitable guide RNA sequences typically comprise guide sequences that are complementary to a nucleic sequence within 50 nucleotides upstream or downstream of the target nucleobase pair to be edited. Some exemplary guide RNA sequences suitable for targeting any of the provided ABEs to specific target sequences are provided herein. Additional guide sequences are well known in the art and may be used with the base editors described herein. Additional exemplary guide sequences are disclosed in, for example, Jinek M., et al., Science 337:816-821(2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are incorporated herein by reference.

### Methods for Making the Adenine Base Editors

The invention further relates in various aspects to methods of making the disclosed improved adenine base editors by various modes of manipulation that include, but are not limited to, codon optimization to achieve greater expression levels in a cell, and the use of nuclear localization sequences (NLSs), preferably at least two NLSs, e.g., two bipartite NLSs, to increase the localization of the expressed base editors into a cell nucleus.

### Preparation of Base Editors for Increased Expression in Cells

The adenine base editors contemplated herein can include modifications that result in increased expression, for example, through codon optimization.

In some embodiments, the base editors (or a component thereof) is codon optimized for expression in particular cells, such as eukaryotic cells. The eukaryotic cells may be those of or derived from a particular organism, such as a mammal, including, but not limited to, human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

The above description is meant to be non-limiting with regard to making base editors having increased expression, and thereby increase editing efficiencies.

### Directed Evolution Methods (e.g., PACE or PANCE)

Various embodiments of the disclosure relate to providing directed evolution methods and systems (e.g., appropriate vectors, cells, phage, flow vessels, etc.) for engineering of the base editors or base editor domains of the present disclosure. The disclosure provides vector systems for the disclosed directed evolution methods to engineer any of the disclosed base editors or base editor fomains (e.g., the adenosine deaminase domains of any of the disclosed base editors).

The directed evolution vector systems and methods provided herein allow for a gene of interest (e.g., a base editor- or adenosine deaminase-encoding gene) in a viral vector to be evolved over multiple generations of viral life cycles in a flow of host cells to acquire a desired function or activity.

Some embodiments of this disclosure provide methods of phage-assisted continuous evolution (PACE) comprising (a) contacting a population of bacterial host cells with a population of bacteriophages that comprise a gene of interest to be evolved and that are deficient in a gene required for the generation of infectious phage, wherein (1) the phage allows for expression of the gene of interest in the host cells; (2) the host cells are suitable host cells for phage infection, replication, and packaging; and (3) the host cells comprise an expression construct encoding the gene required for the generation of infectious phage, wherein expression of the gene is dependent on a function of a gene product of the gene of interest. In some embodiments, the method further comprises (b) incubating the population of host cells under conditions allowing for the mutation of the gene of interest, the production of infectious phage, and the infection of host cells with phage, wherein infected cells are removed from the population of host cells, and wherein the population of host cells is replenished with fresh host cells that have not been infected by the phage. In some embodiments, the method further comprises (c) isolating a mutated phage replication product encoding an evolved protein from the population of host cells.

In PACE, the gene under selection is encoded on the M13 bacteriophage genome. Its activity is linked to M13 propagation by controlling expression of gene III so that only active variants produce infectious progeny phage. Phage are continuously propagated and mutagenized, but mutations accumulate only in the phage genome, not the host or its selection circuit, because fresh host cells are continually flowed into (and out of) the growth vessel, effectively resetting the selection background.

### Development of a PANCE/PACE Evolution Circuit

PACE enables the rapid continuous evolution of biomolecules through many generations of mutation, selection, and replication per day (FIG. 1A)^{12, 13, 29-39}. During PACE, host E. coli cells continuously dilute a population of bacteriophage (selection phage, SP) containing the gene of interest. The gene of interest replaces gene III on the SP, which is required for progeny phage infectivity. SP containing desired gene variants trigger host-cell gene III expression from an accessory plasmid (AP). Host-cell DNA plasmids encode a genetic circuit that links the desired activity of the protein encoded in the SP to the expression of gene III on the AP. Thus, SP variants containing desired gene variants can propagate, while phage encoding inactive variants do not generate infectious progeny and are rapidly diluted out of the culture vessel (or lagoon). An arabinose-inducible mutagenesis plasmid (MP) controls the phage mutation rate.

The key to new PACE selections is linking gene III expression to the activity of interest. A low stringency selection was designed in which base editing activates T7 RNA polymerase, which transcribes gIII. A single editing event can lead to high output amplification immediately upon transcription of the edited DNA. Reference is made to International Patent Publication WO 2019/023680, published Jan. 31, 2019; Badran, A. H. & Liu, D. R. In vivo continuous directed evolution. Curr. Opin. Chem. Biol. 24, 1-10 (2015); Dickinson, B. C., Packer, M. S., Badran, A. H. & Liu, D. R. A system for the continuous directed evolution of proteases rapidly reveals drug-resistance mutations. Nat. Commun. 5, 5352 (2014); Hubbard, B. P. et al. Continuous directed evolution of DNA-binding proteins to improve TALEN specificity. Nat. Methods 12, 939-942 (2015); Wang, T., Badran, A. H., Huang, T. P. & Liu, D. R. Continuous directed evolution of proteins with improved soluble expression. Nat. Chem. Biol. 14, 972-980 (2018), and Thuronyi, B. W. et al. Continuous evolution of base editors with expanded target compatibility and improved activity. Nat. Biotechnol., 1070-1079 (2019), each of which is herein incorporated by reference.

The disclosure provides vector systems for performing directed evolution of adenosine deaminase domains of an adenine base editor. In some embodiments, the vector systems comprise an expression construct that comprises a nucleic acid encoding a split intein portion (e.g., the N-terminal portion or the C-terminal portion of a split intein) operably linked to a nucleic acid encoding a gene required for the production of infectious phage particles, such as gIII protein (pIII protein), or a portion (e.g., fragment) thereof. In some embodiments, the split intein portion is the C-terminal portion of a split intein (e.g., the C-terminal portion of an Npu (Nostoc punctiforme) split intein). In some embodiments, the split intein C-terminal portion is positioned upstream of (e.g., 5′ relative to) the nucleic acid encoding the gene required for the production of infectious phage particles, or portion thereof. In some embodiments, the split intein portion is the N-terminal portion of a split intein (e.g., the N-terminal portion of an Npu split intein). In some embodiments, the split intein N-terminal portion is positioned downstream of (e.g., 3′ relative to) the nucleic acid encoding the gene required for the production of infectious phage particles, or portion thereof. In some embodiments, the disclosed vector system expression constructs (e.g., in a first accessory plasmid or second accessory plasmid) further comprises a sequence encoding luxAB.

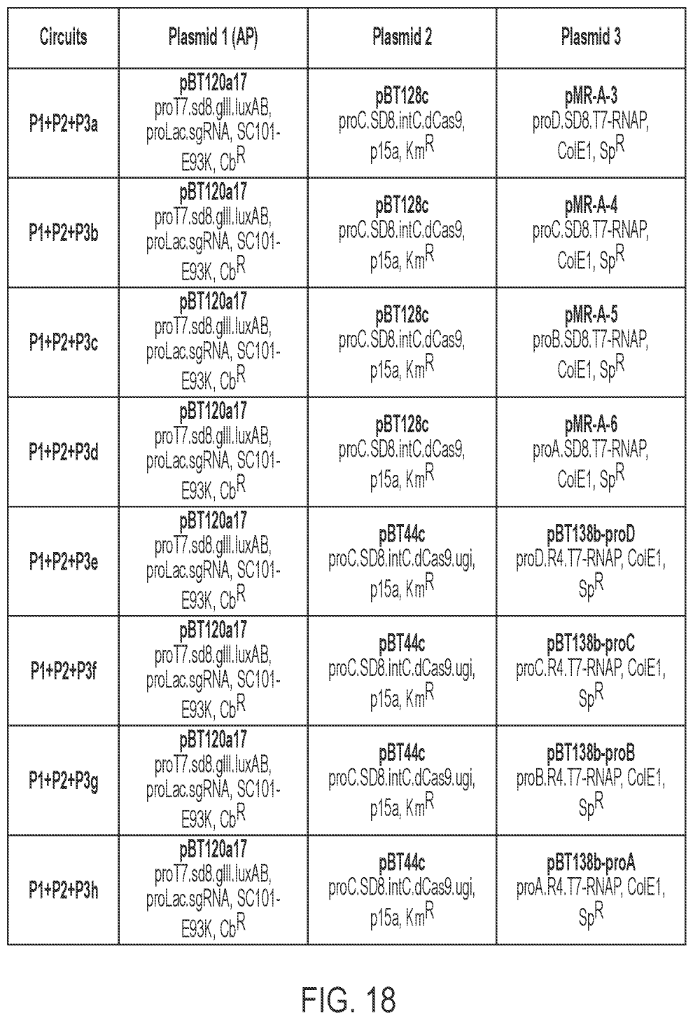

In some embodiments, the vector systems described herein comprising: (i) a selection plasmid comprising an isolated nucleic acid comprising an expression construct encoding an adenosine deaminase comprising, in the following order: an adenosine deaminase protein and a sequence encoding an N-terminal portion of a split intein; (ii) a first accessory plasmid comprising an isolated nucleic acid comprising an expression construct comprising, in the following order: a sequence encoding a guide RNA operably controlled by a Lac promoter and a sequence encoding a M13 phage gIII protein signal peptide operably controlled by a T7 RNA promoter, wherein the sequence encoding the gIII protein signal peptide lacks one or more nucleic acid bases of the signal peptide domain; (iii) a second accessory plasmid comprising an isolated nucleic acid comprising an expression construct comprising, in the following order: a sequence encoding the C-terminal portion of a split intein and a sequence encoding a dCas9; and (iv) a third accessory plasmid comprising an isolated nucleic acid comprising an expression construct comprising, in the following order: a promoter, a ribosome binding site, and a sequence encoding a T7 RNA polymerase (RNAP) comprising mutations that give rise to two stop codons that can be corrected upon adenine base editing (see FIGS. 1B and 18). In some embodiments, the split intein is an Npu split intein. In some embodiments, these stop codons are created at positions 57 and 58. In some embodiments, adenine base editing corrects mutations at positions 57 and 58 in the T7 RNAP coding region and induces substitution back to the wild-type Q57 and R58 (see FIG. 1C). In certain embodiments, the disclosed vector systems further comprise a plurality of third accessory plasmids, each comprising a unique ribosome binding site or a unique promoter. As many as five, six, seven, eight, nine, or ten variants of the third accessory plasmid may be developed with different promoters and ribosome binding sites (RBS) to tune the negative stringency of the PACE evolution. In certain embodiments, the vector systems further comprise a mutagenesis plasmid.

In some embodiments, a vector system is provided as part of a kit, which is useful, in some embodiments, for performing PACE to produce adenosine deaminase protein variants. For example, in some embodiments, a kit comprises a first container housing the selection phagemid of the vector system, a second container housing the first accessory plasmid of the vector system, and a third container housing the second accessory plasmid of the vector system. In some embodiments, a kit further comprises a mutagenesis plasmid. Mutagenesis plasmids for PACE are generally known in the art, and are described, for example in International PCT Application No. PCT/US2016/027795, filed Sep. 16, 2016, published as WO 2016/168631, the entire contents of which are incorporated herein by reference. In some embodiments, the kit further comprises a set of written or electronic instructions for performing PACE.

In some embodiments of the directed evolution methods and systems provided herein, the viral vector or the phage is a filamentous phage, for example, an M13 phage, such as an M13 selection phage as described in more detail elsewhere herein. In some such embodiments, the gene required for the production of infectious viral particles is the M13 gene III (gIII).

In some embodiments, the incubating of the host cells is for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles. In certain embodiments, the viral vector is an M13 phage, and the length of a single viral life cycle is about 10-20 minutes.

In some embodiments, a viral vector/host cell combination is chosen in which the life cycle of the viral vector is significantly shorter than the average time between cell divisions of the host cell. Average cell division times and viral vector life cycle times are well known in the art for many cell types and vectors, allowing those of skill in the art to ascertain such host cell/vector combinations. In certain embodiments, host cells are being removed from the population of host cells contacted with the viral vector at a rate that results in the average time of a host cell remaining in the host cell population before being removed to be shorter than the average time between cell divisions of the host cells, but to be longer than the average life cycle of the viral vector employed. The result of this is that the host cells, on average, do not have sufficient time to proliferate during their time in the host cell population while the viral vectors do have sufficient time to infect a host cell, replicate in the host cell, and generate new viral particles during the time a host cell remains in the cell population. This assures that the only replicating nucleic acid in the host cell population is the viral vector, and that the host cell genome, the accessory plasmid, or any other nucleic acid constructs cannot acquire mutations allowing for escape from the selective pressure imposed.

For example, in some embodiments, the average time a host cell remains in the host cell population is about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes.

In some embodiments, the average time a host cell remains in the host cell population depends on how fast the host cells divide and how long infection (or conjugation) requires. In general, the flow rate should be faster than the average time required for cell division, but slow enough to allow viral (or conjugative) propagation. The former will vary, for example, with the media type, and can be delayed by adding cell division inhibitor antibiotics (FtsZ inhibitors in E. coli, etc.). Since the limiting step in continuous evolution is production of the protein required for gene transfer from cell to cell, the flow rate at which the vector washes out will depend on the current activity of the gene(s) of interest. In some embodiments, titratable production of the protein required for the generation of infectious particles, as described herein, can mitigate this problem. In some embodiments, an indicator of phage infection allows computer-controlled optimization of the flow rate for the current activity level in real-time.

In some embodiments, the fresh host cells comprise the accessory plasmid required for selection of viral vectors, for example, the accessory plasmid comprising the gene required for the generation of infectious phage particles that is lacking from the phages being evolved. In some embodiments, the host cells are generated by contacting an uninfected host cell with the relevant vectors, for example, the accessory plasmid and, optionally, a mutagenesis plasmid, and growing an amount of host cells sufficient for the replenishment of the host cell population in a continuous evolution experiment. Methods for the introduction of plasmids and other gene constructs into host cells are well known to those of skill in the art and the invention is not limited in this respect. For bacterial host cells, such methods include, but are not limited to, electroporation and heat-shock of competent cells.

In some embodiments, the accessory plasmid comprises a selection marker, for example, an antibiotic resistance marker, and the fresh host cells are grown in the presence of the respective antibiotic to ensure the presence of the plasmid in the host cells. Where multiple plasmids are present, different markers are typically used. Such selection markers and their use in cell culture are known to those of skill in the art, and the invention is not limited in this respect.

In particular embodiments, a first accessory plasmid comprises gene III, and a second accessory plasmid comprises a T7 RNAP gene deactivated by a G to T mutation, which results in an early stop codon. A third accessory plasmid may comprise a nucleotide encoding a dCas9 fused at the N terminus to the C-terminal half of a fast-splicing intein. An exemplary phage plasmid may comprise a nucleotide encoding an adenosine deaminase fused at the C terminus to the N-terminal half of the fast-splicing intein. The full-length base editor is reconstituted from the two intein components.

In some embodiments, the selection marker is a spectinomycin antibiotic resistance marker. In other embodiments, the selection marker is a chloramphenicol or carbenicillin resistance marker. Cells may be transformed with a selection plasmid containing an inactivated spectinomycin resistance gene with a mutation at an active site that requires A:T to C:G editing to correct. Cells that fail to install the correct transversion mutation in the spectinomycin resistance gene will die, while cells that make the correction will survive. E. coli cells expressing an sgRNA targeting the active site mutation in the spectinomycin resistance gene and a nucleotide modification domain-dCas9 base editor are plated onto 2×YT agar with 256 μg/mL of spectinomycin. Surviving colonies (measured through CFUs) were sequenced to find consensus mutations in the base editors expressed in the evolved survivors. A similar selection assay was used to evolve adenosine deaminase activity in DNA during adenine base editor development, as described in Gaudelli, N. M. et al., Programmable base editing of A•T to G•C in genomic DNA without DNA cleavage. Nature 551, 464-471 (2017), incorporated herein in its entirety by reference.

In some embodiments, the host cell population in a continuous evolution experiment is replenished with fresh host cells growing in a parallel, continuous culture. In some embodiments, the cell density of the host cells in the host cell population contacted with the viral vector and the density of the fresh host cell population is substantially the same.

Typically, the cells being removed from the cell population contacted with the viral vector comprise cells that are infected with the viral vector and uninfected cells. In some embodiments, cells are being removed from the cell populations continuously, for example, by effecting a continuous outflow of the cells from the population. In other embodiments, cells are removed semi-continuously or intermittently from the population. In some embodiments, the replenishment of fresh cells will match the mode of removal of cells from the cell population, for example, if cells are continuously removed, fresh cells will be continuously introduced. However, in some embodiments, the modes of replenishment and removal may be mismatched, for example, a cell population may be continuously replenished with fresh cells, and cells may be removed semi-continuously or in batches.

In some embodiments, the rate of fresh host cell replenishment and/or the rate of host cell removal is adjusted based on quantifying the host cells in the cell population. For example, in some embodiments, the turbidity of culture media comprising the host cell population is monitored and, if the turbidity falls below a threshold level, the ratio of host cell inflow to host cell outflow is adjusted to effect an increase in the number of host cells in the population, as manifested by increased cell culture turbidity. In other embodiments, if the turbidity rises above a threshold level, the ratio of host cell inflow to host cell outflow is adjusted to effect a decrease in the number of host cells in the population, as manifested by decreased cell culture turbidity. Maintaining the density of host cells in the host cell population within a specific density range ensures that enough host cells are available as hosts for the evolving viral vector population, and avoids the depletion of nutrients at the cost of viral packaging and the accumulation of cell-originated toxins from overcrowding the culture.

In some embodiments, the cell density in the host cell population and/or the fresh host cell density in the inflow is about 10² cells/ml to about 10¹² cells/ml. In some embodiments, the host cell density is about 10² cells/ml, about 10³ cells/ml, about 10⁴ cells/ml, about 10⁵ cells/ml, about 5·10⁵ cells/ml, about 10⁶ cells/ml, about 5.10⁶ cells/ml, about 10⁷ cells/ml, about 5·10⁷ cells/ml, about 10⁸ cells/ml, about 5.10⁸ cells/ml, about 10⁹ cells/ml, about 5·10⁹ cells/ml, about 10¹⁰ cells/ml, or about 5.10¹⁰ cells/ml. In some embodiments, the host cell density is more than about 10¹⁰ cells/ml.

In some embodiments, the host cell population is contacted with a mutagen. In some embodiments, the cell population contacted with the viral vector (e.g., the phage), is continuously exposed to the mutagen at a concentration that allows for an increased mutation rate of the gene of interest, but is not significantly toxic for the host cells during their exposure to the mutagen while in the host cell population. In other embodiments, the host cell population is contacted with the mutagen intermittently, creating phases of increased mutagenesis, and accordingly, of increased viral vector diversification. For example, in some embodiments, the host cells are exposed to a concentration of mutagen sufficient to generate an increased rate of mutagenesis in the gene of interest for about 10%, about 20%, about 50%, or about 75% of the time.

In some embodiments, the host cells comprise a mutagenesis expression construct, for example, in the case of bacterial host cells, a mutagenesis plasmid. In some embodiments, the mutagenesis plasmid comprises a gene expression cassette encoding a mutagenesis-promoting gene product, for example, a proofreading-impaired DNA polymerase. In other embodiments, the mutagenesis plasmid, including a gene involved in the SOS stress response, (e.g., UmuC, UmuD′, and/or RecA). In some embodiments, the mutagenesis-promoting gene is under the control of an inducible promoter. Suitable inducible promoters are well known to those of skill in the art and include, for example, arabinose-inducible promoters, tetracycline or doxycyclin-inducible promoters, and tamoxifen-inducible promoters. In some embodiments, the host cell population is contacted with an inducer of the inducible promoter in an amount sufficient to effect an increased rate of mutagenesis. For example, in some embodiments, a bacterial host cell population is provided in which the host cells comprise a mutagenesis plasmid in which a dnaQ926, UmuC, UmuD′, and RecA expression cassette is controlled by an arabinose-inducible promoter. In some such embodiments, the population of host cells is contacted with the inducer, for example, arabinose in an amount sufficient to induce an increased rate of mutation.

In some embodiments, diversifying the viral vector population is achieved by providing a flow of host cells that does not select for gain-of-function mutations in the gene of interest for replication, mutagenesis, and propagation of the population of viral vectors. In some embodiments, the host cells are host cells that express all genes required for the generation of infectious viral particles, for example, bacterial cells that express a complete helper phage, and, thus, do not impose selective pressure on the gene of interest. In other embodiments, the host cells comprise an accessory plasmid comprising a conditional promoter with a baseline activity sufficient to support viral vector propagation even in the absence of significant gain-of-function mutations of the gene of interest. This can be achieved by using a “leaky” conditional promoter, by using a high-copy number accessory plasmid, thus amplifying baseline leakiness, and/or by using a conditional promoter on which the initial version of the gene of interest effects a low level of activity while a desired gain-of-function mutation effects a significantly higher activity.

Detailed methods of procedures for directing continuous evolution of base editors in a population of host cells using phage particles are disclosed in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; International Application No. PCT/US2019/37216, published as WO 2019/241649 on Dec. 19, 2019, International Patent Publication WO 2019/023680, published Jan. 31, 2019, International PCT Application, PCT/US2016/027795, filed Apr. 15, 2016, published as WO 2016/168631 on Oct. 20, 2016, and International Publication No. WO 2020/041751, published on Feb. 27, 2020, each of which are incorporated herein by reference.

Methods and strategies to design conditional promoters suitable for carrying out the selection strategies described herein are well known to those of skill in the art. For an overview over exemplary suitable selection strategies and methods for designing conditional promoters driving the expression of a gene required for cell-cell gene transfer, e.g., gene III (gIII), see Vidal and Legrain, Yeast n-hybrid review, Nucleic Acid Res. 27, 919 (1999), incorporated herein in its entirety.

The disclosure provides vectors for the continuous evolution processes. In some embodiments, phage vectors for phage-assisted continuous evolution are provided. In some embodiments, a selection phage is provided that comprises a phage genome deficient in at least one gene required for the generation of infectious phage particles and a gene of interest to be evolved. Reference is made to International Patent Publication WO 2019/023680, published Jan. 31, 2019, herein incorporated by reference.

For example, in some embodiments, the selection phage comprises an M13 phage genome deficient in a gene required for the generation of infectious M13 phage particles, for example, a full-length gIII. In some embodiments, the selection phage comprises a phage genome providing all other phage functions required for the phage life cycle except the gene required for generation of infectious phage particles. In some such embodiments, an M13 selection phage is provided that comprises a gI, gII, gIV, gV, gVI, gVII, gVIII, gIX, and a gX gene, but not a full-length gIII. In some embodiments, the selection phage comprises a 3′-fragment of gIII, but no full-length gIII. The 3′-end of gIII comprises a promoter and retaining this promoter activity is beneficial, in some embodiments, for an increased expression of gVI, which is immediately downstream of the gIII 3′-promoter, or a more balanced (wild-type phage-like) ratio of expression levels of the phage genes in the host cell, which, in turn, can lead to more efficient phage production. In some embodiments, the 3′-fragment of gIII gene comprises the 3′-gIII promoter sequence. In some embodiments, the 3′-fragment of gIII comprises the last 180 bp, the last 150 bp, the last 125 bp, the last 100 bp, the last 50 bp, or the last 25 bp of gIII. In some embodiments, the 3′-fragment of gIII comprises the last 180 bp of gIII.

M13 selection phage is provided that comprises a gene of interest in the phage genome, for example, inserted downstream of the gVIII 3′-terminator and upstream of the gIII-3′-promoter. In some embodiments, an M13 selection phage is provided that comprises a multiple cloning site for cloning a gene of interest into the phage genome, for example, a multiple cloning site (MCS) inserted downstream of the gVIII 3′-terminator and upstream of the gIII-3′-promoter.

Some embodiments of this disclosure provide a vector system for continuous evolution procedures, comprising of a viral vector, for example, a selection phage, and a matching accessory plasmid. In some embodiments, a vector system for phage-based continuous directed evolution is provided that comprises (a) a selection phage comprising a gene of interest to be evolved, wherein the phage genome is deficient in a gene required to generate infectious phage; and (b) an accessory plasmid comprising the gene required to generate infectious phage particle under the control of a conditional promoter, wherein the conditional promoter is activated by a function of a gene product encoded by the gene of interest.

In some embodiments, the selection phage is an M13 phage as described herein. For example, in some embodiments, the selection phage comprises an M13 genome including all genes required for the generation of phage particles, for example, gI, gII, gIV, gV, gVI, gVII, gVIII, gIX, and gX gene, but not a full-length gIII gene. In some embodiments, the selection phage genome comprises an F1 or an M13 origin of replication. In some embodiments, the selection phage genome comprises a 3′-fragment of gIII gene. In some embodiments, the selection phage comprises a multiple cloning site upstream of the gIII 3′-promoter and downstream of the gVIII 3′-terminator.

In an exemplary PACE methodology, host cells each containing a mutagenesis plasmid are diluted into 5 mL Davis Rich Medium (DRM) with appropriate antibiotics and grown to an A600 of 0.4-0.8. Cells are then used to inoculate a chemostat (60 mL), which may be maintained under continuous dilution with fresh DRM at 1-1.5 volumes per hour to keep cell density roughly constant. Lagoons are initially filled with DRM, then continuously diluted with chemostat culture for at least 2 hours before seeding with phage. A stock solution of arabinose (1 M) may be pumped directly into lagoons (10 mM final) as previously described³⁹ for 1 hour before the addition of selection phage (SP). For the first 12 hours after phage inoculation, anhydrotetracycline is present in the stock solution (3.3 μg/mL). Lagoons may be seeded at a starting titer of ˜10⁷ pfu per mL. Dilution rate may be adjusted by modulating lagoon volume (5-20 mL) and/or culture inflow rate (10-20 mL/h). Lagoons may be sampled every 24 hours by removal of culture (500 μL) by syringe. Samples are centrifuged at 13,500 g for 2 minutes and the supernatant removed and stored at 4° C. Titers are evaluated by plaquing. The presence of T7 RNAP or gene III recombinant phage is monitored by plaquing on S2060 cells containing pT7-AP and no plasmid. Phage genotypes may be assessed from single plaques by diagnostic PCR.

Some embodiments of this disclosure provide a method of non-continuous evolution of a gene of interest. In certain embodiments, the method of non-continuous evolution is PANCE. In other embodiments, the method of non-continuous evolution is an antibiotic or plate-based selection method. PANCE uses the same genetic circuit as PACE to activate phage propagation, but instead of continuously diluting a vessel, phage are manually passaged by infecting fresh host-cell culture with an aliquot from the proceeding passage. PANCE is less stringent than PACE because there is little risk of losing a weakly active phage variant during selection, and because the effective rate of phage dilution is much lower.

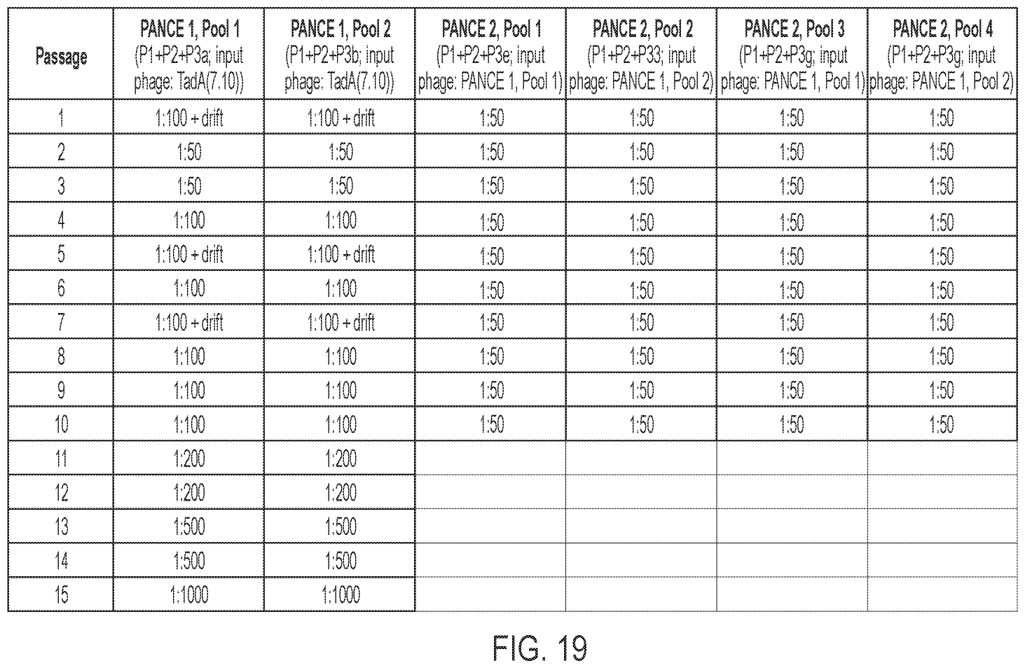

An exemplary PANCE methododology comprises first growing the host strain containing a mutagenesis plasmid of E. coli on 2×YT agar containing 0.5% glucose (w/v) along with appropriate concentrations of antibiotics until optical density reaches A600=0.5-0.6 in a large volume. The cells are re-transformed with the mutagenesis plasmid regularly to ensure the plasmid has not been inactivated. An aliquot of a desired concentration, often 2 mL, is then transferred to a smaller flask, supplemented with 40 mM inducing agent arabinose (Ara) for the mutagenesis plasmid, and infected with the selection phage (SP). To increase the titer level, a drift plasmid may also be provided that enables phage to propagate without passing the selection. Expression is under the control of an inducible promoter and can be turned on with 0-40 ng/mL of anhydrotetracycline. Treated cultures may be split into the desired number of either 2 mL cultures in single culture tubes or 500 μL cultures in a 96-well plate and infected with selection phage (see FIG. 19). These cultures may be incubated at 37° C. for 8-12 h to facilitate phage growth, which is confirmed by determination of the phage titer, and then harvested. Following phage growth, an aliquot of infected cells is used to transfect a subsequent flask containing host E. coli. Supernatant containing evolved phage may isolated and stored at 4° C. This process may be continued until the desired phenotype is evolved for as many transfers as required, while increasing the stringency in stepwise fashion by decreasing the incubation time or titer of phage with which the bacteria is infected. In an exemplary PANCE protocol as provided herein, the process is iterated in 25 culture passages. Reference is made to Suzuki T. et al., Crystal structures reveal an elusive functional domain of pyrrolysyl-tRNA synthetase, Nat Chem Biol. 13(12): 1261-1266 (2017), incorporated herein in its entirety.

In some embodiments, negative selection is applied during a non-continuous evolution method as described herein, by penalizing undesired activities. In some embodiments, this is achieved by causing the undesired activity to interfere with pIII production. For example, expression of an antisense RNA complementary to the gIII RBS and/or start codon is one way of applying negative selection, while expressing a protease (e.g., TEV) and engineering the protease recognition sites into pIII is another.

Other non-continuous selection schemes for gene products having a desired activity are well known to those of skill in the art or will be apparent from the present disclosure. In certain embodiments, following the successful directed evolution of one or more components of the adenine base editor (e.g., a Cas9 domain or a adenosine deaminase domain), methods of making the base editors comprise recombinant protein expression methodologies known to one of ordinary skill in the art.

### Vectors

Several aspects of the making and using the base editors of the disclosure relate to vector systems comprising one or more vectors encoding the adenine base editors. Vectors may be designed to clone and/or express the adenine base editors of the disclosure. Vectors may also be designed to transfect the adenine base editors of the disclosure into one or more cells, e.g., a target diseased eukaryotic cell for treatment with the base editor systems and methods disclosed herein.

Vectors may be designed for expression of base editor transcripts (e.g. nucleic acid transcripts, proteins, or enzymes) in prokaryotic or eukaryotic cells. For example, base editor transcripts may be expressed in bacterial cells such as Escherichia coli, insect cells (using baculovirus expression vectors), yeast cells, plant cells, or mammalian cells. Suitable host cells are discussed further in Goeddel, Gene Expression Technology: Methods In Enzymology 185, Academic Press. San Diego, Calif. (1990). Alternatively, expression vectors encoding one or more adenine base editors described herein may be transcribed and translated in vitro, for example using T7 promoter regulatory sequences and T7 polymerase. Vectors encoding the adenine base editors provided herein may comprise any of the DNA plasmids identified with the “A-to-G base editor” purpose provided at the Addgene webpage, https://www.addgene.org/browse/article/28207557/. Exemplary vectors include the ABE8e, SaABE8e, SaKKH-ABE8e, CP1028-ABE8e, and NG-ABE8e vectors.

Vectors may be introduced and propagated in a prokaryotic cells. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-base editors.

Fusion expression vectors also may be used to express the adenine base editors of the disclosure. Such vectors generally add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the base editor. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

### Eukaryotic Cell Systems for Determining Off-Target Effects of Base Editors

In some aspects, eukaryotic cell assays and systems for measuring off-target effects (e.g., off-target editing frequencies) of an adenine base editor are provided. These systems may be used in accordance with the disclosed methods. These systems are referred to in the Examples as an “orthogonal R-loop assay.” Systems for determining the off-target editing frequency of a base editor may comprise one or more eukaryotic cells each comprising i) a first nucleic acid molecule encoding a base editor comprising a napDNAbp domain; (ii) a second nucleic acid molecule encoding a first guide RNA that is engineered to bind to the napDNAbp domain of the base editor, wherein the first guide RNA comprises a first sequence of at least 10 contiguous nucleotides that is complementary to a target sequence; (iii) a third nucleic acid molecule encoding a nuclease inactive napDNAbp protein; and (iv) a fourth nucleic acid molecule encoding a second gRNA that is engineered to bind to the nuclease inactive napDNAbp protein, wherein the second guide RNA comprises a second sequence of at least 10 contiguous nucleotides that is complementary to a third sequence, whereby the first complex and second complex generate two or more R-loops, and wherein the third sequence has about 60% or less sequence identity to the target sequence. Exemplary eukaryotic cell assays and systems for measuring off-target effects of the disclosed adenine base editors are disclosed in and International Application No. PCT/US2020/062428, filed Nov. 25, 2020, incorporate herein by reference.

The disclosed systems may further comprise a third, fourth, fifth, and/or sixth complex, wherein each of the third, fourth, fifth, and/or sixth complexes comprises (v) a second nuclease inactive napDNAbp protein, and (vi) a third guide RNA that is engineered to bind to the second nuclease inactive napDNAbp protein, wherein the third guide RNA comprises a fourth sequence of at least 10 contiguous nucleotides that is complementary to the third sequence. These complexes may be identical or essentially identical to each other, in that they are associated with identical or nearly identical gRNAs that have complementarity to the same off-target sequence. Any one of these complexes may be distinct or essentially identical to the second complex. The second and third guide RNA may share at least 95%, 98%, 98.5%, or 100% sequence identity, e.g., in the backbone of the guide RNA sequence. In certain embodiments, the second and third guide RNA share 100% identity or are the same. Likewise, the first nuclease inactive napDNAbp protein and the second nuclease inactive napDNAbp may be the same.

In some embodiments, any of the nuclease inactive napDNAbp proteins of the described systems may be a dead Cas9 (dCas9) protein. Accordingly, in some embodiments, the second complex comprises a first dCas9 protein, and the third and subsequent complexes comprise a second dCas9 protein. In some embodiments, the nuclease inactive napDNAbp protein of any of the described complexes is a dead Cas9 protein from S. aureus. In some embodiments, the nuclease inactive napDNAbp protein is a dead Cas9 protein from S. pyogenes.

In some embodiments, the eukaryotic cells of the disclosed systems comprise mammalian cells. The eukaryotic cells may comprise human cells, e.g. HEK293T cells.

In some embodiments of these methods, transformed eukaryotic cells are sequenced to validate that mutations arise from adenine-to-guanine conversions. This sequencing step may be achieved by Sanger sequencing, high-throughput sequencing, whole genome sequencing, and/or other sequencing methods known in the art.

The on-target and Cas9-independent off-target editing rates of various base editors, such as ABEs, may be compared by transforming any one of the disclosed eukaryotic cell systems with plasmids encoding these base editors in parallel, and evaluating the deamination rates at on-target and off-target sites for each base editor.

### Methods of Editing A Target Nucleobase Pair, Methods of Treatment, and Uses of the Adenine Base Editors

Some aspects of the disclosure provide methods for editing a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to an adenosine deaminase domain) and a guide nucleic acid (e.g., gRNA), wherein the target region comprises a targeted nucleobase pair. As a result of embodiments of these methods, strand separation of said target region is induced, a first nucleobase of said target nucleobase pair in a single strand of the target region is converted to a second nucleobase, and no more than one strand of said target region is cut (or nicked), wherein a third nucleobase complementary to the first nucleobase base is replaced by a fourth nucleobase complementary to the second nucleobase.

In some embodiments, the first nucleobase is an adenine. In some embodiments, the second nucleobase is a deaminated adenine, or hypoxanthine. In some embodiments, the third nucleobase is a thymine. In some embodiments, the fourth nucleobase is a cytosine. In some embodiments, the method further comprises replacing the second nucleobase with a fifth nucleobase that is complementary to the fourth nucleobase, thereby generating an intended edited base pair (e.g., A:T to G:C). In some embodiments, the fifth nucleobase is a guanine. In some embodiments, at least 5% of the intended base pairs are edited. In some embodiments, at least 10%, 15%, 20%, 25%, 30%, 35%, 40%, 45%, or 50% of the intended base pairs are edited.

In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is adenine, and the second base is not a G, C, A, or T. In some embodiments, the second base is hypoxanthine. In some embodiments, the first base is adenine. In some embodiments, the second base is not a G, C, A, or T. In some embodiments, the second base is hypoxanthine. In some embodiments, the base editor inhibits base excision repair of the edited strand. In some embodiments, the base editor protects or binds the non-edited strand. In some embodiments, the base editor comprises a catalytically inactive hypoxanthine-specific nuclease. In some embodiments, the base editor comprises nickase activity. In some embodiments, the intended edited base pair is upstream of a PAM site.

In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited basepair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the base editor comprises a linker. In some embodiments, the linker is 1-25 amino acids in length. In some embodiments, the linker is 5-20 amino acids in length. In some embodiments, linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the adenine base editors provided herein. In some embodiments, a target window is a deamination window.

In some aspects, the disclosure provides improved adenine base editors with expanded target windows. In some embodiments, the target window of the disclosed base editors corresponds to protospacer positions 4-8 of the target sequence, wherein protospacer position 0 corresponds to the position of the first contiguous nucleotide of the guide RNA sequence that is complementary to the target sequence, or to the position of the transcription start site of the target gene. Protospacer position 0 may also refer to the nucleotide position most distal from the PAM. In other embodiments of the disclosed base editors, the base editors have an expanded target window that corresponds to protospacer positions 3-14 of the target sequence relative to the position of the transcription start site of the target gene. In other embodiments, the target window corresponds to protospacer positions 4-11. In still other embodiments, the target window corresponds to protospacer positions 8-14. In still other embodiments, the target window corresponds to protospacer positions 9-14. (See FIG. 2G.) In some embodiments, the target window is in a gene (e.g. HBG, HBB, or BC11A). In some embodiments, the base editors with wider target windows comprise CP1028-ABE8e (set forth in SEQ ID NO: 183). In some embodiments, the base editors with wider target windows comprise CP1041-ABE8e (SEQ ID NO: 185).

In some embodiments, the method comprises a) contacting a target region of the double-stranded DNA sequence with a complex comprising a base editor and a guide nucleic acid (e.g., gRNA), where the target region comprises a target nucleobase pair, and thereby inducing strand separation of said target region, converting a first nucleobase of said target nucleobase pair in a single strand of the target region to a second nucleobase, cutting no more than one strand of said target region, wherein a third nucleobase complementary to the first nucleobase base is replaced by a fourth nucleobase complementary to the second nucleobase, and the second nucleobase is replaced with a fifth nucleobase that is complementary to the fourth nucleobase, and thereby generating an intended edited base pair, wherein the efficiency of generating the intended edited base pair is at least 5%. In some embodiments, at least 5% of the intended base pairs are edited. In some embodiments, at least 10%, 15%, 20%, 25%, 30%, 35%, 40%, 45%, or 50% of the intended base pairs are edited. In some embodiments, the ratio of intended product to unintended products at the target nucleotide is at least 2:1, 5:1, 10:1, 20:1, 30:1, 40:1, 50:1, 60:1, 70:1, 80:1, 90:1, 100:1, or 200:1, or more. In some embodiments, the cut single strand is hybridized to the guide nucleic acid. In some embodiments, the cut single strand is opposite to the strand comprising the first nucleobase. In some embodiments, the first base is adenine. In some embodiments, the second nucleobase is not G, C, A, or T. In some embodiments, the second base is hypoxanthine. In some embodiments, the base editor inhibits base excision repair of the edited strand.

In other embodiments, the disclosure provides editing methods comprising contacting a DNA, or RNA molecule with any of the adenine base editors provided herein, and with at least one guide nucleic acid (e.g., guide RNA), wherein the guide nucleic acid, (e.g., guide RNA) is about 15-100 nucleotides long and comprises a sequence of at least 10 contiguous nucleotides that is complementary to a target sequence. In some embodiments, the 3′ end of the target sequence is immediately adjacent to a canonical PAM sequence (NGG). In some embodiments, the 3′ end of the target sequence is not immediately adjacent to a canonical PAM sequence (NGG). In some embodiments, the 3′ end of the target sequence is immediately adjacent to an AGC, GAG, TTT, GTG, or CAA sequence. In some embodiments, the 3′ end of the target sequence is immediately adjacent to a non-canonical PAM sequence (e.g., NGN).

In some embodiments, the target DNA sequence comprises a sequence associated with a disease or disorder. In some embodiments, the target DNA sequence comprises a point mutation associated with a disease or disorder. In some embodiments, the activity of the base editor (e.g., comprising an adenosine deaminase and a Cas9 domain), or the complex, results in a correction of the point mutation. In some embodiments, the target DNA sequence comprises a G→A point mutation associated with a disease or disorder, and wherein the deamination of the mutant A base results in a sequence that is not associated with a disease or disorder. In some embodiments, the target DNA sequence encodes a protein, and the point mutation is in a codon and results in a change in the amino acid encoded by the mutant codon as compared to the wild-type codon. In some embodiments, the deamination of the mutant A results in a change of the amino acid encoded by the mutant codon. In some embodiments, the deamination of the mutant A results in the codon encoding the wild-type amino acid. In some embodiments, the contacting is in vivo in a subject. In some embodiments, the subject has or has been diagnosed with a disease or disorder. Multiplexed Base Editing Applications

In some aspects, the present disclosure provides methods of editing two or more nucleic acid target sites using the disclosed adenine base editors simultaneously. In multiplexed base editing of unique genomic loci, a plurality of gRNAs having complementarity to different target sequences enables the formation of base editor-gRNA complexes at each of several (e.g. 5, 10, 15, 20, 25, or more) target sequences simulataneously, or within a single iteration or cycle.

The discovery and widespread implementation of the CRISPR/Cas system has dramatically expanded the toolbox for genome engineering and has revolutionized the future prospects of basic biological research, data storage in living systems, agricultural science, and medicine. An advantage of CRISPR/Cas-based genome editors over prior approaches is the capacity to multiplex by using several guide RNAs (gRNAs). This not only enables the screening of libraries of guides in a single cell population but also the targeting of up to six unique loci at once. However, the editing efficiency at each site tends to decrease when compared to that of a single guide transfection.

The present disclosure provides for methods of base editing comprising: contacting a nucleic acid molecule (e.g. DNA) with a plurality of complexes, wherein each complex comprises a base editor and a guide RNA (gRNA) bound to the napDNAbp domain of the base editor, wherein at least two of the complexes of the plurality each comprise a unique gRNA comprising a guide sequence of at least 10 contiguous nucleotides that is complementary to a unique target sequence in the genomic DNA of a cell. In certain embodiments, the cell is a eukaryotic cell, e.g. a mammalian cell. In certain embodiments, the cell is a human cell. In certain embodiments, the plurality of the disclosed base editor-gRNA complexes make simultaneous edits (i.e., within a single iteration) at various target loci within a eukaryotic cell, e.g. a mammalian cell.

In some embodiments, any of the target sequences of these multiplexed editing methods comprises a genomic locus. In some embodiments, the multiple target sequences comprise unique genomic loci. In some embodiments, at least one of the target sequences comprises a sequence in an HBG promoter or the BCL11A enhancer. In some embodiments, at least one of the target sequences comprises a sequence in the HBB gene.

In some embodiments, at least three, at least four, or at least five of the complexes of the plurality each comprise a unique gRNA comprising a guide sequence of at least 10 contiguous nucleotides that is complementary to a unique target sequence in the genomic DNA of a cell, e.g. a eukaryotic cell.

In some embodiments, the base editing (deamination) efficiency at each unique target sequence is substantially equivalent to that of a single guide transfection at each of these sequences. In certain embodiments, the step of contacting results in an average base editing efficiency of at least 20%, 22%, 24%, 26%, 28%, 30%, 40%, or 50% across the aggregate of all unique target sequences. In some embodiments, the step of contacting results in an average efficiency of about 24%. In some embodiments, this average efficiency of about 24% is substantially equivalent to that of a single guide transfection at each of these sequences.

Any of the base editor-gRNA complexes provided herein may be introduced into the cell for multiplexed base editing in any suitable way, either stably or transiently. In some embodiments, a base editor may be transfected into the cell. In some embodiments, the cell may be transduced or transfected with a nucleic acid construct that encodes the base editor. For example, a cell may be transduced (e.g. with a virus encoding a base editor) or transfected (e.g. with a plasmid encoding a base editor) with a nucleic acid that encodes the base editor. Alternatively, a cell may be introduced with the base editor itself. Such transduction may be a stable or transient transduction. In some embodiments, cells expressing a base editing base editor, or comprising a base editor, may be transduced or transfected with one or more gRNA molecules, for example, when the base editor comprises a Cas9 (e.g., nCas9) domain. In some embodiments, a plasmid expressing a base editor may be introduced into cells through electroporation (e.g., using an ATX MaxCyte electroporator), transient transfection (e.g. lipofection) or stable genome integration (e.g., piggybac), viral transduction, or other methods known to those of skill in the art.

In certain embodiments of the disclosed methods, the constructs that encode the base editors are transfected into the cell separately from the constructs that encode the gRNAs. In certain embodiments, these components are encoded on a single construct and transfected together. In particular embodiments, these single constructs encoding the base editors and gRNAs may be transfected into the cell iteratively, with each iteration associated with a subset of target sequences. In particular embodiments, these single constructs may be transfected into the cell over a period of days. In other embodiments, they may be transfected into the cell over a period of hours. In other embodiments, they may be transected into the cell over a period of weeks.

In the disclosed methods, target cells may be incubated with the base editor-gRNA complexes for two days, or 48 hours, after transfection to achieve multiplexed base editing. Target cells may be incubated for 30 hours, 40 hours, 54 hours, 60 hours, or 72 hours after transfection. Target cells may be incubated with the base editor-gRNA complexes for four days, five days, seven days, nine days, eleven days, or thirteen days or more after transfection.

In some aspects, the disclosure provides pharmaceutical compositions comprising a plurality of any of the base editors described herein and a gRNA, wherein at least five of the base editors of the plurality are each bound to a unique gRNA, and a pharmaceutically acceptable excipient.

### Methods of Treatment

The present disclosure provides methods for the treatment of a subject diagnosed with a disease associated with or caused by a point mutation that may be corrected by a DNA editing base editor provided herein. For example, in some embodiments, a method is provided that comprises administering to a subject having such a disease, e.g., a cancer associated with a point mutation as described above, an effective amount of an adenosine deaminase base editor that corrects the point mutation or introduces a deactivating mutation into a disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that may be treated by correcting a point mutation or introducing a deactivating mutation into a disease-associated gene will be known to those of skill in the art, and the disclosure is not limited in this respect.

In some embodiments, the deamination of the mutant A results in the codon encoding the wild-type amino acid. In some embodiments, the contacting is in vivo in a subject. In some embodiments, the subject has or has been diagnosed with a disease or disorder. In some embodiments, the disease or disorder is a hemoglobinopathy. In some embodiments, the disease or disorder is sickle cell disease. In some embodiments, the disease or disorder is β-thalassemia. In some embodiments, the disease or disorder is Glycogen storage disease type 1A, which is associated with a R83C mutation in the Glucose-6-phosphatase-alpha (G6PC) enzyme, and Stargardt macular dystrophy, which is associated with a G1961E mutation in the ATP-binding cassette, sub-family A, member 4 (ABCA4) protein. A list of additional exemplary diseases and disorders that may be treated using the base editors described herein is shown in Table 4. In some embodiments, the disease or disorder is phenylketonuria, von Willebrand disease (vWD), a neoplastic disease associated with a mutant PTEN or BRCA1, or Li-Fraumeni syndrome. Table 4 includes the target gene, the mutation to be corrected, the related disease and the nucleotide sequence of the associated protospacer and PAM.

TABLE 4
List of exemplary diseases that may be treated using the base editors described herein.

| | | ATCC Cell | | |
|---|---|---|---|---|
| Target Gene | Mutation | Line | Disease | Protospacer and PAM |
| PTEN | Cys136Tyr | HTP-20 | Cancer Predisposition | TATATGCATATTTATTACATCGG (SEQ ID NO: 85) |
| PTEN | Arg233Ter | HTB-13 | Cancer Predisposition | CCGTCATGTGGGTCCTGAATTGG (SEQ ID NO: 86) |
| TP53 | Glu258Lys | HTB-65 | Cancer Predisposition | ACACTGAAAGACTCCAGGTCAGG (SEQ ID NO: 87) |
| BRCA1 | Gly1738Arg | NA | Cancer Predisposition | GTCAGAAGAGATGTGGTCAATGG (SEQ ID NO: 88) |
| BRCA1 | 4097-1G > A | NA | Cancer Predisposition | TTTAAAGTGAAGCAGCATCTGGG (SEQ ID NO: 89) |
| | | | | ATTTAAAGTGAAGCAGCATCTGG {SEQ ID NO: 90) |
| PAH | Thr380Mer | NA | Phenylketonuria | ACTCCATGACAGTGTAATTTTGG (SEQ ID NO: 91) |
| VWF | Ser1285Phe | NA | von Willebrand | GCCTGGAGAAGCCATCCAGCAGG (SEQ ID NO: 92) |
| | | | (Hemophilia) | |
| VWF | Arg253STer | NA | von Willebrand | CTCAGACACACTCATTGATGAGG (SEQ ID NO: 93) |
| | | | (Hemophilia) | |
| TP53 | Arg175His | HCC1395 | L1-Fraumeni syndrome | GAGGCACTGCCCCCACCATGAGCG (SEQ ID NO: 94) |

The Adenine to be edited in the protospacer is indicated by underlining and the PAM is indicated in bold.

Some embodiments provide methods for using the adenine base editors provided herein. In some embodiments, the base editors are used to introduce a point mutation into a nucleic acid by deaminating a target nucleobase, e.g., an A residue. In some embodiments, the deamination of the target nucleobase results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, in some embodiments, methods are provided herein that employ a DNA editing base editor to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the purpose of the methods provided herein is to restore the function of a dysfunctional gene via genome editing. The nucleobase editing proteins provided herein can be validated for gene editing-based human therapeutics in vitro, e.g., by correcting a disease-associated mutation in human cell culture. It will be understood by the skilled artisan that the nucleobase editing proteins provided herein, e.g., the base editors comprising a nucleic acid programmable DNA binding protein (e.g., Cas9) and an adenosine deaminase domain may be used to correct any single point G to A or C to T mutation. In the first case, deamination of the mutant A to I corrects the mutation, and in the latter case, deamination of the A that is base-paired with the mutant T, followed by a round of replication, corrects the mutation. Exemplary point mutations that may be corrected are listed in Table 4.

The successful correction of point mutations in disease-associated genes and alleles opens up new strategies for gene correction with applications in therapeutics and basic research. Site-specific single-base modification systems like the disclosed fusions of a napDNAbp domain and an adenosine deaminase domain also have applications in “reverse” gene therapy, where certain gene functions are purposely suppressed or abolished. In these cases, site-specifically mutating residues that lead to inactivating mutations in a protein, or mutations that inhibit function of the protein may be used to abolish or inhibit protein function. Without wishing to be bound by any particular theory certain anemias, such as sickle cell anemia, may be treated by inducing expression of hemoglobin, such as fetal hemoglobin, which is typically silenced in adults. As one example, mutating −198T to C in the promoter driving HBG1 and HBG2 gene expression results in increased expression of HBG1 and HBG2. As another example, a class of disorders that results from a G to A mutation in a gene is iron storage disorders, where the HFE gene comprises a G to A mutation that results in expression of a C282Y mutant HFE protein, may be treated using any of the adenine base editors provided herein. As another example, a mutation in the HBB gene that causes the sickle cell disease allele, HBB^{S}, may be mutated to a non-pathogenic allele, such as the naturally-occurring Makassar (HBB^{G}) allele using any of the disclosed base editors. As such, correction of the point mutation results in a conversion of an HBB^{S} allele to an HBB^{G} allele.

The present disclosure provides methods for the treatment of additional diseases or disorders, e.g., diseases or disorders that are associated or caused by a point mutation that may be corrected by deaminase-mediated gene editing. Some such diseases are described herein, and additional suitable diseases that may be treated with the strategies and base editors provided herein will be apparent to those of skill in the art based on the present disclosure. Exemplary suitable diseases and disorders are listed below. Exemplary suitable diseases and disorders include, without limitation: 2-methyl-3-hydroxybutyric aciduria; 3 beta-Hydroxysteroid dehydrogenase deficiency; 3-Methylglutaconic aciduria; 3-Oxo-5 alpha-steroid delta 4-dehydrogenase deficiency; 46,XY sex reversal, type 1, 3, and 5; 5-Oxoprolinase deficiency; 6-pyruvoyl-tetrahydropterin synthase deficiency; Aarskog syndrome; Aase syndrome; Achondrogenesis type 2; Achromatopsia 2 and 7; Acquired long QT syndrome; Acrocallosal syndrome, Schinzel type; Acrocapitofemoral dysplasia; Acrodysostosis 2, with or without hormone resistance; Acroerythrokeratoderma; Acromicric dysplasia; Acth-independent macronodular adrenal hyperplasia 2; Activated PI3K-delta syndrome; Acute intermittent porphyria; deficiency of Acyl-CoA dehydrogenase family, member 9; Adams-Oliver syndrome 5 and 6; Adenine phosphoribosyltransferase deficiency; Adenylate kinase deficiency; hemolytic anemia due to Adenylosuccinate lyase deficiency; Adolescent nephronophthisis; Renal-hepatic-pancreatic dysplasia; Meckel syndrome type 7; Adrenoleukodystrophy; Adult junctional epidermolysis bullosa; Epidermolysis bullosa, junctional, localisata variant; Adult neuronal ceroid lipofuscinosis; Adult neuronal ceroid lipofuscinosis; Adult onset ataxia with oculomotor apraxia; ADULT syndrome; Afibrinogenemia and congenital Afibrinogenemia; autosomal recessive Agammaglobulinemia 2; Age-related macular degeneration 3, 6, 11, and 12; Aicardi Goutieres syndromes 1, 4, and 5; Chilbain lupus 1; Alagille syndromes 1 and 2; Alexander disease; Alkaptonuria; Allan-Herndon-Dudley syndrome; Alopecia universalis congenital; Alpers encephalopathy; Alpha-1-antitrypsin deficiency; autosomal dominant, autosomal recessive, and X-linked recessive Alport syndromes; Alzheimer disease, familial, 3, with spastic paraparesis and apraxia; Alzheimer disease, types, 1, 3, and 4; hypocalcification type and hypomaturation type, IIA1 Amelogenesis imperfecta; Aminoacylase 1 deficiency; Amish infantile epilepsy syndrome; Amyloidogenic transthyretin amyloidosis; Amyloid Cardiomyopathy, Transthyretin-related; Cardiomyopathy; Amyotrophic lateral sclerosis types 1, 6, 15 (with or without frontotemporal dementia), 22 (with or without frontotemporal dementia), and 10; Frontotemporal dementia with TDP43 inclusions, TARDBP-related; Andermann syndrome; Andersen Tawil syndrome; Congenital long QT syndrome; Anemia, nonspherocytic hemolytic, due to G6PD deficiency; Angelman syndrome; Severe neonatal-onset encephalopathy with microcephaly; susceptibility to Autism, X-linked 3; Angiopathy, hereditary, with nephropathy, aneurysms, and muscle cramps; Angiotensin i-converting enzyme, benign serum increase; Aniridia, cerebellar ataxia, and mental retardation; Anonychia; Antithrombin III deficiency; Antley-Bixler syndrome with genital anomalies and disordered steroidogenesis; Aortic aneurysm, familial thoracic 4, 6, and 9; Thoracic aortic aneurysms and aortic dissections; Multisystemic smooth muscle dysfunction syndrome; Moyamoya disease 5; Aplastic anemia; Apparent mineralocorticoid excess; Arginase deficiency; Argininosuccinate lyase deficiency; Aromatase deficiency; Arrhythmogenic right ventricular cardiomyopathy types 5, 8, and 10; Primary familial hypertrophic cardiomyopathy; Arthrogryposis multiplex congenita, distal, X-linked; Arthrogryposis renal dysfunction cholestasis syndrome; Arthrogryposis, renal dysfunction, and cholestasis 2; Asparagine synthetase deficiency; Abnormality of neuronal migration; Ataxia with vitamin E deficiency; Ataxia, sensory, autosomal dominant; Ataxia-telangiectasia syndrome; Hereditary cancer-predisposing syndrome; Atransferrinemia; Atrial fibrillation, familial, 11, 12, 13, and 16; Atrial septal defects 2, 4, and 7 (with or without atrioventricular conduction defects); Atrial standstill 2; Atrioventricular septal defect 4; Atrophia bulborum hereditaria; ATR-X syndrome; Auriculocondylar syndrome 2; Autoimmune disease, multisystem, infantile-onset; Autoimmune lymphoproliferative syndrome, type 1a; Autosomal dominant hypohidrotic ectodermal dysplasia; Autosomal dominant progressive external ophthalmoplegia with mitochondrial DNA deletions 1 and 3; Autosomal dominant torsion dystonia 4; Autosomal recessive centronuclear myopathy; Autosomal recessive congenital ichthyosis 1, 2, 3, 4A, and 4B; Autosomal recessive cutis laxa type IA and 1B; Autosomal recessive hypohidrotic ectodermal dysplasia syndrome; Ectodermal dysplasia 11b; hypohidrotic/hair/tooth type, autosomal recessive; Autosomal recessive hypophosphatemic bone disease; Axenfeld-Rieger syndrome type 3; Bainbridge-Ropers syndrome; Bannayan-Riley-Ruvalcaba syndrome; PTEN hamartoma tumor syndrome; Baraitser-Winter syndromes 1 and 2; Barakat syndrome; Bardet-Biedl syndromes 1, 11, 16, and 19; Bare lymphocyte syndrome type 2, complementation group E; Bartter syndrome antenatal type 2; Bartter syndrome types 3, 3 with hypocalciuria, and 4; Basal ganglia calcification, idiopathic, 4; Beaded hair; Benign familial hematuria; Benign familial neonatal seizures 1 and 2; Seizures, benign familial neonatal, 1, and/or myokymia; Seizures, Early infantile epileptic encephalopathy 7; Benign familial neonatal-infantile seizures; Benign hereditary chorea; Benign scapuloperoneal muscular dystrophy with cardiomyopathy; Bernard-Soulier syndrome, types A1 and A2 (autosomal dominant); Bestrophinopathy, autosomal recessive; beta Thalassemia; Bethlem myopathy and Bethlem myopathy 2; Bietti crystalline corneoretinal dystrophy; Bile acid synthesis defect, congenital, 2; Biotinidase deficiency; Birk Barel mental retardation dysmorphism syndrome; Blepharophimosis, ptosis, and epicanthus inversus; Bloom syndrome; Borjeson-Forssman-Lehmann syndrome; Boucher Neuhauser syndrome; Brachydactyly types A1 and A2; Brachydactyly with hypertension; Brain small vessel disease with hemorrhage; Branched-chain ketoacid dehydrogenase kinase deficiency; Branchiootic syndromes 2 and 3; Breast cancer, early-onset; Breast-ovarian cancer, familial 1, 2, and 4; Brittle cornea syndrome 2; Brody myopathy; Bronchiectasis with or without elevated sweat chloride 3; Brown-Vialetto-Van laere syndrome and Brown-Vialetto-Van Laere syndrome 2; Brugada syndrome; Brugada syndrome 1; Ventricular fibrillation; Paroxysmal familial ventricular fibrillation; Brugada syndrome and Brugada syndrome 4; Long QT syndrome; Sudden cardiac death; Bull eye macular dystrophy; Stargardt disease 4; Cone-rod dystrophy 12; Bullous ichthyosiform erythroderma; Burn-Mckeown syndrome; Candidiasis, familial, 2, 5, 6, and 8; Carbohydrate-deficient glycoprotein syndrome type I and II; Carbonic anhydrase VA deficiency, hyperammonemia due to; Carcinoma of colon; Cardiac arrhythmia; Long QT syndrome, LQT1 subtype; Cardioencephalomyopathy, fatal infantile, due to cytochrome c oxidase deficiency; Cardiofaciocutaneous syndrome; Cardiomyopathy; Danon disease; Hypertrophic cardiomyopathy; Left ventricular noncompaction cardiomyopathy; Carnevale syndrome; Carney complex, type 1; Carnitine acylcarnitine translocase deficiency; Carnitine palmitoyltransferase I, II, II (late onset), and II (infantile) deficiency; Cataract 1, 4, autosomal dominant, autosomal dominant, multiple types, with microcornea, coppock-like, juvenile, with microcornea and glucosuria, and nuclear diffuse nonprogressive; Catecholaminergic polymorphic ventricular tachycardia; Caudal regression syndrome; Cd8 deficiency, familial; Central core disease; Centromeric instability of chromosomes 1,9 and 16 and immunodeficiency; Cerebellar ataxia infantile with progressive external ophthalmoplegi and Cerebellar ataxia, mental retardation, and dysequilibrium syndrome 2; Cerebral amyloid angiopathy, APP-related; Cerebral autosomal dominant and recessive arteriopathy with subcortical infarcts and leukoencephalopathy; Cerebral cavernous malformations 2; Cerebrooculofacioskeletal syndrome 2; Cerebro-oculo-facio-skeletal syndrome; Cerebroretinal microangiopathy with calcifications and cysts; Ceroid lipofuscinosis neuronal 2, 6, 7, and 10; Ch\xc3\xa9diak-Higashi syndrome, Chediak-Higashi syndrome, adult type; Charcot-Marie-Tooth disease types 1B, 2B2, 2C, 2F, 2I, 2U (axonal), 1C (demyelinating), dominant intermediate C, recessive intermediate A, 2A2, 4C, 4D, 4H, IF, IVF, and X; Scapuloperoneal spinal muscular atrophy; Distal spinal muscular atrophy, congenital nonprogressive; Spinal muscular atrophy, distal, autosomal recessive, 5; CHARGE association; Childhood hypophosphatasia; Adult hypophosphatasia; Cholecystitis; Progressive familial intrahepatic cholestasis 3; Cholestasis, intrahepatic, of pregnancy 3; Cholestanol storage disease; Cholesterol monooxygenase (side-chain cleaving) deficiency; Chondrodysplasia Blomstrand type; Chondrodysplasia punctata 1, X-linked recessive and 2 X-linked dominant; CHOPS syndrome; Chronic granulomatous disease, autosomal recessive cytochrome b-positive, types 1 and 2; Chudley-McCullough syndrome; Ciliary dyskinesia, primary, 7, 11, 15, 20 and 22; Citrullinemia type I; Citrullinemia type I and II; Cleidocranial dysostosis; C-like syndrome; Cockayne syndrome type A, Coenzyme Q10 deficiency, primary 1, 4, and 7; Coffin Siris/Intellectual Disability; Coffin-Lowry syndrome; Cohen syndrome, Cold-induced sweating syndrome 1; COLE-CARPENTER SYNDROME 2; Combined cellular and humoral immune defects with granulomas; Combined d-2- and 1-2-hydroxyglutaric aciduria; Combined malonic and methylmalonic aciduria; Combined oxidative phosphorylation deficiencies 1, 3, 4, 12, 15, and 25; Combined partial and complete 17-alpha-hydroxylase/17,20-lyase deficiency; Common variable immunodeficiency 9; Complement component 4, partial deficiency of, due to dysfunctional c1 inhibitor; Complement factor B deficiency; Cone monochromatism; Cone-rod dystrophy 2 and 6; Cone-rod dystrophy amelogenesis imperfecta; Congenital adrenal hyperplasia and Congenital adrenal hypoplasia, X-linked; Congenital amegakaryocytic thrombocytopenia; Congenital aniridia; Congenital central hypoventilation; Hirschsprung disease 3; Congenital contractural arachnodactyly; Congenital contractures of the limbs and face, hypotonia, and developmental delay; Congenital disorder of glycosylation types 1B, 1D, 1G, 1H, 1J, 1K, 1N, 1P, 2C, 2J, 2K, IIm; Congenital dyserythropoietic anemia, type I and II; Congenital ectodermal dysplasia of face; Congenital erythropoietic porphyria; Congenital generalized lipodystrophy type 2; Congenital heart disease, multiple types, 2; Congenital heart disease; Interrupted aortic arch; Congenital lipomatous overgrowth, vascular malformations, and epidermal nevi; Non-small cell lung cancer; Neoplasm of ovary; Cardiac conduction defect, nonspecific; Congenital microvillous atrophy; Congenital muscular dystrophy; Congenital muscular dystrophy due to partial LAMA2 deficiency; Congenital muscular dystrophy-dystroglycanopathy with brain and eye anomalies, types A2, A7, A8, All, and A14; Congenital muscular dystrophy-dystroglycanopathy with mental retardation, types B2, B3, B5, and B15; Congenital muscular dystrophy-dystroglycanopathy without mental retardation, type B5; Congenital muscular hypertrophy-cerebral syndrome; Congenital myasthenic syndrome, acetazolamide-responsive; Congenital myopathy with fiber type disproportion; Congenital ocular coloboma; Congenital stationary night blindness, type 1A, 1B, 1C, 1E, 1F, and 2A; Coproporphyria; Cornea plana 2; Corneal dystrophy, Fuchs endothelial, 4; Corneal endothelial dystrophy type 2; Corneal fragility keratoglobus, blue sclerae and joint hypermobility; Cornelia de Lange syndromes 1 and 5; Coronary artery disease, autosomal dominant 2; Coronary heart disease; Hyperalphalipoproteinemia 2; Cortical dysplasia, complex, with other brain malformations 5 and 6; Cortical malformations, occipital; Corticosteroid-binding globulin deficiency; Corticosterone methyloxidase type 2 deficiency; Costello syndrome; Cowden syndrome 1; Coxa plana; Craniodiaphyseal dysplasia, autosomal dominant; Craniosynostosis 1 and 4; Craniosynostosis and dental anomalies; Creatine deficiency, X-linked; Crouzon syndrome; Cryptophthalmos syndrome; Cryptorchidism, unilateral or bilateral; Cushing symphalangism; Cutaneous malignant melanoma 1; Cutis laxa with osteodystrophy and with severe pulmonary, gastrointestinal, and urinary abnormalities; Cyanosis, transient neonatal and atypical nephropathic; Cystic fibrosis; Cystinuria; Cytochrome c oxidase i deficiency; Cytochrome-c oxidase deficiency; D-2-hydroxyglutaric aciduria 2; Darier disease, segmental; Deafness with labyrinthine aplasia microtia and microdontia (LAMM); Deafness, autosomal dominant 3a, 4, 12, 13, 15, autosomal dominant nonsyndromic sensorineural 17, 20, and 65; Deafness, autosomal recessive 1A, 2, 3, 6, 8, 9, 12, 15, 16, 18b, 22, 28, 31, 44, 49, 63, 77, 86, and 89; Deafness, cochlear, with myopia and intellectual impairment, without vestibular involvement, autosomal dominant, X-linked 2; Deficiency of 2-methylbutyryl-CoA dehydrogenase; Deficiency of 3-hydroxyacyl-CoA dehydrogenase; Deficiency of alpha-mannosidase; Deficiency of aromatic-L-amino-acid decarboxylase; Deficiency of bisphosphoglycerate mutase; Deficiency of butyryl-CoA dehydrogenase; Deficiency of ferroxidase; Deficiency of galactokinase; Deficiency of guanidinoacetate methyltransferase; Deficiency of hyaluronoglucosaminidase; Deficiency of ribose-5-phosphate isomerase; Deficiency of steroid 11-beta-monooxygenase; Deficiency of UDPglucose-hexose-1-phosphate uridylyltransferase; Deficiency of xanthine oxidase; Dejerine-Sottas disease; Charcot-Marie-Tooth disease, types ID and IVF; Dejerine-Sottas syndrome, autosomal dominant; Dendritic cell, monocyte, B lymphocyte, and natural killer lymphocyte deficiency; Desbuquois dysplasia 2; Desbuquois syndrome; DFNA 2 Nonsyndromic Hearing Loss; Diabetes mellitus and insipidus with optic atrophy and deafness; Diabetes mellitus, type 2, and insulin-dependent, 20; Diamond-Blackfan anemia 1, 5, 8, and 10; Diarrhea 3 (secretory sodium, congenital, syndromic) and 5 (with tufting enteropathy, congenital); Dicarboxylic aminoaciduria; Diffuse palmoplantar keratoderma, Bothnian type; Digitorenocerebral syndrome; Dihydropteridine reductase deficiency; Dilated cardiomyopathy 1A, 1AA, 1C, 1G, 1BB, 1DD, 1FF, 1HH, 1I, 1KK, 1N, 1S, 1Y, and 3B; Left ventricular noncompaction 3; Disordered steroidogenesis due to cytochrome p450 oxidoreductase deficiency; Distal arthrogryposis type 2B; Distal hereditary motor neuronopathy type 2B; Distal myopathy Markesbery-Griggs type; Distal spinal muscular atrophy, X-linked 3; Distichiasis-lymphedema syndrome; Dominant dystrophic epidermolysis bullosa with absence of skin; Dominant hereditary optic atrophy; Donnai Barrow syndrome; Dopamine beta hydroxylase deficiency; Dopamine receptor d2, reduced brain density of; Dowling-degos disease 4; Doyne honeycomb retinal dystrophy; Malattia leventinese; Duane syndrome type 2; Dubin-Johnson syndrome; Duchenne muscular dystrophy; Becker muscular dystrophy; Dysfibrinogenemia; Dyskeratosis congenita autosomal dominant and autosomal dominant, 3; Dyskeratosis congenita, autosomal recessive, 1, 3, 4, and 5; Dyskeratosis congenita X-linked; Dyskinesia, familial, with facial myokymia; Dysplasminogenemia; Dystonia 2 (torsion, autosomal recessive), 3 (torsion, X-linked), 5 (Dopa-responsive type), 10, 12, 16, 25, 26 (Myoclonic); Seizures, benign familial infantile, 2; Early infantile epileptic encephalopathy 2, 4, 7, 9, 10, 11, 13, and 14; Atypical Rett syndrome; Early T cell progenitor acute lymphoblastic leukemia; Ectodermal dysplasia skin fragility syndrome; Ectodermal dysplasia-syndactyly syndrome 1; Ectopia lentis, isolated autosomal recessive and dominant; Ectrodactyly, ectodermal dysplasia, and cleft lip/palate syndrome 3; Ehlers-Danlos syndrome type 7 (autosomal recessive), classic type, type 2 (progeroid), hydroxylysine-deficient, type 4, type 4 variant, and due to tenascin-X deficiency; Eichsfeld type congenital muscular dystrophy; Endocrine-cerebroosteodysplasia; Enhanced s-cone syndrome; Enlarged vestibular aqueduct syndrome; Enterokinase deficiency; Epidermodysplasia verruciformis; Epidermolysa bullosa simplex and limb girdle muscular dystrophy, simplex with mottled pigmentation, simplex with pyloric atresia, simplex, autosomal recessive, and with pyloric atresia; Epidermolytic palmoplantar keratoderma; Familial febrile seizures 8; Epilepsy, childhood absence 2, 12 (idiopathic generalized, susceptibility to) 5 (nocturnal frontal lobe), nocturnal frontal lobe type 1, partial, with variable foci, progressive myoclonic 3, and X-linked, with variable learning disabilities and behavior disorders; Epileptic encephalopathy, childhood-onset, early infantile, 1, 19, 23, 25, 30, and 32; Epiphyseal dysplasia, multiple, with myopia and conductive deafness; Episodic ataxia type 2; Episodic pain syndrome, familial, 3; Epstein syndrome; Fechtner syndrome; Erythropoietic protoporphyria; Estrogen resistance; Exudative vitreoretinopathy 6; Fabry disease and Fabry disease, cardiac variant; Factor H, VII, X, v and factor viii, combined deficiency of 2, xiii, a subunit, deficiency; Familial adenomatous polyposis 1 and 3; Familial amyloid nephropathy with urticaria and deafness; Familial cold urticarial; Familial aplasia of the vermis; Familial benign pemphigus; Familial cancer of breast; Breast cancer, susceptibility to; Osteosarcoma; Pancreatic cancer 3; Familial cardiomyopathy; Familial cold autoinflammatory syndrome 2; Familial colorectal cancer; Familial exudative vitreoretinopathy, X-linked; Familial hemiplegic migraine types 1 and 2; Familial hypercholesterolemia; Familial hypertrophic cardiomyopathy 1, 2, 3, 4, 7, 10, 23 and 24; Familial hypokalemia-hypomagnesemia; Familial hypoplastic, glomerulocystic kidney; Familial infantile myasthenia; Familial juvenile gout; Familial Mediterranean fever and Familial mediterranean fever, autosomal dominant; Familial porencephaly; Familial porphyria cutanea tarda; Familial pulmonary capillary hemangiomatosis; Familial renal glucosuria; Familial renal hypouricemia; Familial restrictive cardiomyopathy 1; Familial type 1 and 3 hyperlipoproteinemia; Fanconi anemia, complementation group E, I, N, and O; Fanconi-Bickel syndrome; Favism, susceptibility to; Febrile seizures, familial, 11; Feingold syndrome 1; Fetal hemoglobin quantitative trait locus 1; FG syndrome and FG syndrome 4; Fibrosis of extraocular muscles, congenital, 1, 2, 3a (with or without extraocular involvement), 3b; Fish-eye disease; Fleck corneal dystrophy; Floating-Harbor syndrome; Focal epilepsy with speech disorder with or without mental retardation; Focal segmental glomerulosclerosis 5; Forebrain defects; Frank Ter Haar syndrome; Borrone Di Rocco Crovato syndrome; Frasier syndrome; Wilms tumor 1; Freeman-Sheldon syndrome; Frontometaphyseal dysplasia land 3; Frontotemporal dementia; Frontotemporal dementia and/or amyotrophic lateral sclerosis 3 and 4; Frontotemporal Dementia Chromosome 3-Linked and Frontotemporal dementia ubiquitin-positive; Fructose-biphosphatase deficiency; Fuhrmann syndrome; Gamma-aminobutyric acid transaminase deficiency; Gamstorp-Wohlfart syndrome; Gaucher disease type 1 and Subacute neuronopathic; Gaze palsy, familial horizontal, with progressive scoliosis; Generalized dominant dystrophic epidermolysis bullosa; Generalized epilepsy with febrile seizures plus 3, type 1, type 2; Epileptic encephalopathy Lennox-Gastaut type; Giant axonal neuropathy; Glanzmann thrombasthenia; Glaucoma 1, open angle, e, F, and G; Glaucoma 3, primary congenital, d; Glaucoma, congenital and Glaucoma, congenital, Coloboma; Glaucoma, primary open angle, juvenile-onset; Glioma susceptibility 1; Glucose transporter type 1 deficiency syndrome; Glucose-6-phosphate transport defect; GLUT1 deficiency syndrome 2; Epilepsy, idiopathic generalized, susceptibility to, 12; Glutamate formiminotransferase deficiency; Glutaric acidemia IIA and IIB; Glutaric aciduria, type 1; Gluthathione synthetase deficiency; Glycogen storage disease 0 (muscle), II (adult form), IXa2, IXc, type 1A; type II, type IV, IV (combined hepatic and myopathic), type V, and type VI; Goldmann-Favre syndrome; Gordon syndrome; Gorlin syndrome; Holoprosencephaly sequence; Holoprosencephaly 7; Granulomatous disease, chronic, X-linked, variant; Granulosa cell tumor of the ovary; Gray platelet syndrome; Griscelli syndrome type 3; Groenouw corneal dystrophy type I; Growth and mental retardation, mandibulofacial dysostosis, microcephaly, and cleft palate; Growth hormone deficiency with pituitary anomalies; Growth hormone insensitivity with immunodeficiency; GTP cyclohydrolase I deficiency; Haj du-Cheney syndrome; Hand foot uterus syndrome; Hearing impairment; Hemangioma, capillary infantile; Hematologic neoplasm; Hemochromatosis type 1, 2B, and 3; Microvascular complications of diabetes 7; Transferrin serum level quantitative trait locus 2; Hemoglobin H disease, nondeletional; Hemolytic anemia, nonspherocytic, due to glucose phosphate isomerase deficiency; Hemophagocytic lymphohistiocytosis, familial, 2; Hemophagocytic lymphohistiocytosis, familial, 3; Heparin cofactor II deficiency; Hereditary acrodermatitis enteropathica; Hereditary breast and ovarian cancer syndrome; Ataxia-telangiectasia-like disorder; Hereditary diffuse gastric cancer; Hereditary diffuse leukoencephalopathy with spheroids; Hereditary factors II, IX, VIII deficiency disease; Hereditary hemorrhagic telangiectasia type 2; Hereditary insensitivity to pain with anhidrosis; Hereditary lymphedema type I; Hereditary motor and sensory neuropathy with optic atrophy; Hereditary myopathy with early respiratory failure; Hereditary neuralgic amyotrophy; Hereditary Nonpolyposis Colorectal Neoplasms; Lynch syndrome I and II; Hereditary pancreatitis; Pancreatitis, chronic, susceptibility to; Hereditary sensory and autonomic neuropathy type IIB and IIA; Hereditary sideroblastic anemia; Hermansky-Pudlak syndrome 1, 3, 4, and 6; Heterotaxy, visceral, 2, 4, and 6, autosomal; Heterotaxy, visceral, X-linked; Heterotopia; Histiocytic medullary reticulosis; Histiocytosis-lymphadenopathy plus syndrome; Holocarboxylase synthetase deficiency; Holoprosencephaly 2, 3, 7, and 9; Holt-Oram syndrome; Homocysteinemia due to MTHFR deficiency, CBS deficiency, and Homocystinuria, pyridoxine-responsive; Homocystinuria-Megaloblastic anemia due to defect in cobalamin metabolism, cblE complementation type; Howel-Evans syndrome; Hurler syndrome; Hutchinson-Gilford syndrome; Hydrocephalus; Hyperammonemia, type III; Hypercholesterolaemia and Hypercholesterolemia, autosomal recessive; Hyperekplexia 2 and Hyperekplexia hereditary; Hyperferritinemia cataract syndrome; Hyperglycinuria; Hyperimmunoglobulin D with periodic fever; Mevalonic aciduria; Hyperimmunoglobulin E syndrome; Hyperinsulinemic hypoglycemia familial 3, 4, and 5; Hyperinsulinism-hyperammonemia syndrome; Hyperlysinemia; Hypermanganesemia with dystonia, polycythemia and cirrhosis; Hyperornithinemia-hyperammonemia-homocitrullinuria syndrome; Hyperparathyroidism 1 and 2; Hyperparathyroidism, neonatal severe; Hyperphenylalaninemia, bh4-deficient, a, due to partial pts deficiency, BH4-deficient, D, and non-pku; Hyperphosphatasia with mental retardation syndrome 2, 3, and 4; Hypertrichotic osteochondrodysplasia; Hypobetalipoproteinemia, familial, associated with apob32; Hypocalcemia, autosomal dominant 1; Hypocalciuric hypercalcemia, familial, types 1 and 3; Hypochondrogenesis; Hypochromic microcytic anemia with iron overload; Hypoglycemia with deficiency of glycogen synthetase in the liver; Hypogonadotropic hypogonadism 11 with or without anosmia; Hypohidrotic ectodermal dysplasia with immune deficiency; Hypohidrotic X-linked ectodermal dysplasia; Hypokalemic periodic paralysis 1 and 2; Hypomagnesemia 1, intestinal; Hypomagnesemia, seizures, and mental retardation; Hypomyelinating leukodystrophy 7; Hypoplastic left heart syndrome; Atrioventricular septal defect and common atrioventricular junction; Hypospadias 1 and 2, X-linked; Hypothyroidism, congenital, nongoitrous, 1; Hypotrichosis 8 and 12; Hypotrichosis-lymphedema-telangiectasia syndrome; I blood group system; Ichthyosis bullosa of Siemens; Ichthyosis exfoliativa; Ichthyosis prematurity syndrome; Idiopathic basal ganglia calcification 5; Idiopathic fibrosing alveolitis, chronic form; Dyskeratosis congenita, autosomal dominant, 2 and 5; Idiopathic hypercalcemia of infancy; Immune dysfunction with T-cell inactivation due to calcium entry defect 2; Immunodeficiency 15, 16, 19, 30, 31C, 38, 40, 8, due to defect in cd3-zeta, with hyper IgM type 1 and 2, and X-Linked, with magnesium defect, Epstein-Barr virus infection, and neoplasia; Immunodeficiency-centromeric instability-facial anomalies syndrome 2; Inclusion body myopathy 2 and 3; Nonaka myopathy; Infantile convulsions and paroxysmal choreoathetosis, familial; Infantile cortical hyperostosis; Infantile GM1 gangliosidosis; Infantile hypophosphatasia; Infantile nephronophthisis; Infantile nystagmus, X-linked; Infantile Parkinsonism-dystonia; Infertility associated with multi-tailed spermatozoa and excessive DNA; Insulin resistance; Insulin-resistant diabetes mellitus and acanthosis nigricans; Insulin-dependent diabetes mellitus secretory diarrhea syndrome; Interstitial nephritis, karyomegalic; Intrauterine growth retardation, metaphyseal dysplasia, adrenal hypoplasia congenita, and genital anomalies; Iodotyrosyl coupling defect; IRAK4 deficiency; Iridogoniodysgenesis dominant type and type 1; Iron accumulation in brain; Ischiopatellar dysplasia; Islet cell hyperplasia; Isolated 17,20-lyase deficiency; Isolated lutropin deficiency; Isovaleryl-CoA dehydrogenase deficiency; Jankovic Rivera syndrome; Jervell and Lange-Nielsen syndrome 2; Joubert syndrome 1, 6, 7, 9/15 (digenic), 14, 16, and 17, and Orofaciodigital syndrome xiv; Junctional epidermolysis bullosa gravis of Herlitz; Juvenile GM>1<gangliosidosis; Juvenile polyposis syndrome; Juvenile polyposis/hereditary hemorrhagic telangiectasia syndrome; Juvenile retinoschisis; Kabuki make-up syndrome; Kallmann syndrome 1, 2, and 6; Delayed puberty; Kanzaki disease; Karak syndrome; Kartagener syndrome; Kenny-Caffey syndrome type 2; Keppen-Lubinsky syndrome; Keratoconus 1; Keratosis follicularis; Keratosis palmoplantaris striata 1; Kindler syndrome; L-2-hydroxyglutaric aciduria; Larsen syndrome, dominant type; Lattice corneal dystrophy Type III; Leber amaurosis; Zellweger syndrome; Peroxisome biogenesis disorders; Zellweger syndrome spectrum; Leber congenital amaurosis 11, 12, 13, 16, 4, 7, and 9; Leber optic atrophy; Aminoglycoside-induced deafness; Deafness, nonsyndromic sensorineural, mitochondrial; Left ventricular noncompaction 5; Left-right axis malformations; Leigh disease; Mitochondrial short-chain Enoyl-CoA Hydratase 1 deficiency; Leigh syndrome due to mitochondrial complex I deficiency; Leiner disease; Leri Weill dyschondrosteosis; Lethal congenital contracture syndrome 6; Leukocyte adhesion deficiency type I and III; Leukodystrophy, Hypomyelinating, 11 and 6; Leukoencephalopathy with ataxia, with Brainstem and Spinal Cord Involvement and Lactate Elevation, with vanishing white matter, and progressive, with ovarian failure; Leukonychia totalis; Lewy body dementia; Lichtenstein-Knorr Syndrome; Li-Fraumeni syndrome 1; Lig4 syndrome; Limb-girdle muscular dystrophy, type 1B, 2A, 2B, 2D, C1, C5, C9, C14; Congenital muscular dystrophy-dystroglycanopathy with brain and eye anomalies, type A14 and B14; Lipase deficiency combined; Lipid proteinosis; Lipodystrophy, familial partial, type 2 and 3; Lissencephaly 1, 2 (X-linked), 3, 6 (with microcephaly), X-linked; Subcortical laminar heterotopia, X-linked; Liver failure acute infantile; Loeys-Dietz syndrome 1, 2, 3; Long QT syndrome 1, 2, 2/9, 2/5, (digenic), 3, 5 and 5, acquired, susceptibility to; Lung cancer; Lymphedema, hereditary, id; Lymphedema, primary, with myelodysplasia; Lymphoproliferative syndrome 1, 1 (X-linked), and 2; Lysosomal acid lipase deficiency; Macrocephaly, macrosomia, facial dysmorphism syndrome; Macular dystrophy, vitelliform, adult-onset; Malignant hyperthermia susceptibility type 1; Malignant lymphoma, non-Hodgkin; Malignant melanoma; Malignant tumor of prostate; Mandibuloacral dysostosis; Mandibuloacral dysplasia with type A or B lipodystrophy, atypical; Mandibulofacial dysostosis, Treacher Collins type, autosomal recessive; Mannose-binding protein deficiency; Maple syrup urine disease type 1A and type 3; Marden Walker like syndrome; Marfan syndrome; Marinesco-Sj\xc3\xb6gren syndrome; Martsolf syndrome; Maturity-onset diabetes of the young, type 1, type 2, type 11, type 3, and type 9; May-Hegglin anomaly; MYH9 related disorders; Sebastian syndrome; McCune-Albright syndrome; Somatotroph adenoma; Sex cord-stromal tumor; Cushing syndrome; McKusick Kaufman syndrome; McLeod neuroacanthocytosis syndrome; Meckel-Gruber syndrome; Medium-chain acyl-coenzyme A dehydrogenase deficiency; Medulloblastoma; Megalencephalic leukoencephalopathy with subcortical cysts land 2a; Megalencephaly cutis marmorata telangiectatica congenital; PIK3CA Related Overgrowth Spectrum; Megalencephaly-polymicrogyria-polydactyly-hydrocephalus syndrome 2; Megaloblastic anemia, thiamine-responsive, with diabetes mellitus and sensorineural deafness; Meier-Gorlin syndromes land 4; Melnick-Needles syndrome; Meningioma; Mental retardation, X-linked, 3, 21, 30, and 72; Mental retardation and microcephaly with pontine and cerebellar hypoplasia; Mental retardation X-linked syndromic 5; Mental retardation, anterior maxillary protrusion, and strabismus; Mental retardation, autosomal dominant 12, 13, 15, 24, 3, 30, 4, 5, 6, and 9; Mental retardation, autosomal recessive 15, 44, 46, and 5; Mental retardation, stereotypic movements, epilepsy, and/or cerebral malformations; Mental retardation, syndromic, Claes-Jensen type, X-linked; Mental retardation, X-linked, nonspecific, syndromic, Hedera type, and syndromic, wu type; Merosin deficient congenital muscular dystrophy; Metachromatic leukodystrophy juvenile, late infantile, and adult types; Metachromatic leukodystrophy; Metatrophic dysplasia; Methemoglobinemia types I and 2; Methionine adenosyltransferase deficiency, autosomal dominant; Methylmalonic acidemia with homocystinuria, Methylmalonic aciduria cb1B type, Methylmalonic aciduria due to methylmalonyl-CoA mutase deficiency; METHYLMALONIC ACIDURIA, mut(0) TYPE; Microcephalic osteodysplastic primordial dwarfism type 2; Microcephaly with or without chorioretinopathy, lymphedema, or mental retardation; Microcephaly, hiatal hernia and nephrotic syndrome; Microcephaly; Hypoplasia of the corpus callosum; Spastic paraplegia 50, autosomal recessive; Global developmental delay; CNS hypomyelination; Brain atrophy; Microcephaly, normal intelligence and immunodeficiency; Microcephaly-capillary malformation syndrome; Microcytic anemia; Microphthalmia syndromic 5, 7, and 9; Microphthalmia, isolated 3, 5, 6, 8, and with coloboma 6; Microspherophakia; Migraine, familial basilar; Miller syndrome; Minicore myopathy with external ophthalmoplegia; Myopathy, congenital with cores; Mitchell-Riley syndrome; mitochondrial 3-hydroxy-3-methylglutaryl-CoA synthase deficiency; Mitochondrial complex I, II, III, III (nuclear type 2, 4, or 8) deficiency; Mitochondrial DNA depletion syndrome 11, 12 (cardiomyopathic type), 2, 4B (MNGIE type), 8B (MNGIE type); Mitochondrial DNA-depletion syndrome 3 and 7, hepatocerebral types, and 13 (encephalomyopathic type); Mitochondrial phosphate carrier and pyruvate carrier deficiency; Mitochondrial trifunctional protein deficiency; Long-chain 3-hydroxyacyl-CoA dehydrogenase deficiency; Miyoshi muscular dystrophy 1; Myopathy, distal, with anterior tibial onset; Mohr-Tranebjaerg syndrome; Molybdenum cofactor deficiency, complementation group A; Mowat-Wilson syndrome; Mucolipidosis III Gamma; Mucopolysaccharidosis type VI, type VI (severe), and type VII; Mucopolysaccharidosis, MPS-I-HIS, MPS-II, MPS-III-A, MPS-III-B, MPS-III-C, MPS-IV-A, MPS—IV-B; Retinitis Pigmentosa 73; Gangliosidosis GM1 type1 (with cardiac involvement) 3; Multicentric osteolysis nephropathy; Multicentric osteolysis, nodulosis and arthropathy; Multiple congenital anomalies; Atrial septal defect 2; Multiple congenital anomalies-hypotonia-seizures syndrome 3; Multiple Cutaneous and Mucosal Venous Malformations; Multiple endocrine neoplasia, types land 4; Multiple epiphyseal dysplasia 5 or Dominant; Multiple gastrointestinal atresias; Multiple pterygium syndrome Escobar type; Multiple sulfatase deficiency; Multiple synostoses syndrome 3; Muscle AMP deaminase deficiency; Muscle eye brain disease; Muscular dystrophy, congenital, megaconial type; Myasthenia, familial infantile, 1; Myasthenic Syndrome, Congenital, 11, associated with acetylcholine receptor deficiency; Myasthenic Syndrome, Congenital, 17, 2A (slow-channel), 4B (fast-channel), and without tubular aggregates; Myeloperoxidase deficiency; MYH-associated polyposis; Endometrial carcinoma; Myocardial infarction 1; Myoclonic dystonia; Myoclonic-Atonic Epilepsy; Myoclonus with epilepsy with ragged red fibers; Myofibrillar myopathy 1 and ZASP-related; Myoglobinuria, acute recurrent, autosomal recessive; Myoneural gastrointestinal encephalopathy syndrome; Cerebellar ataxia infantile with progressive external ophthalmoplegia; Mitochondrial DNA depletion syndrome 4B, MNGIE type; Myopathy, centronuclear, 1, congenital, with excess of muscle spindles, distal, 1, lactic acidosis, and sideroblastic anemia 1, mitochondrial progressive with congenital cataract, hearing loss, and developmental delay, and tubular aggregate, 2; Myopia 6; Myosclerosis, autosomal recessive; Myotonia congenital; Congenital myotonia, autosomal dominant and recessive forms; Nail-patella syndrome; Nance-Horan syndrome; Nanophthalmos 2; Navajo neurohepatopathy; Nemaline myopathy 3 and 9; Neonatal hypotonia; Intellectual disability; Seizures; Delayed speech and language development; Mental retardation, autosomal dominant 31; Neonatal intrahepatic cholestasis caused by citrin deficiency; Nephrogenic diabetes insipidus, Nephrogenic diabetes insipidus, X-linked; Nephrolithiasis/osteoporosis, hypophosphatemic, 2; Nephronophthisis 13, 15 and 4; Infertility; Cerebello-oculo-renal syndrome (nephronophthisis, oculomotor apraxia and cerebellar abnormalities); Nephrotic syndrome, type 3, type 5, with or without ocular abnormalities, type 7, and type 9; Nestor-Guillermo progeria syndrome; Neu-Laxova syndrome 1; Neurodegeneration with brain iron accumulation 4 and 6; Neuroferritinopathy; Neurofibromatosis, type land type 2; Neurofibrosarcoma; Neurohypophyseal diabetes insipidus; Neuropathy, Hereditary Sensory, Type IC; Neutral 1 amino acid transport defect; Neutral lipid storage disease with myopathy; Neutrophil immunodeficiency syndrome; Nicolaides-Baraitser syndrome; Niemann-Pick disease type C1, C2, type A, and type C1, adult form; Non-ketotic hyperglycinemia; Noonan syndrome 1 and 4, LEOPARD syndrome 1; Noonan syndrome-like disorder with or without juvenile myelomonocytic leukemia; Normokalemic periodic paralysis, potassium-sensitive; Norum disease; Epilepsy, Hearing Loss, And Mental Retardation Syndrome; Mental Retardation, X-Linked 102 and syndromic 13; Obesity; Ocular albinism, type I; Oculocutaneous albinism type 1B, type 3, and type 4; Oculodentodigital dysplasia; Odontohypophosphatasia; Odontotrichomelic syndrome; Oguchi disease; Oligodontia-colorectal cancer syndrome; Opitz G/BBB syndrome; Optic atrophy 9; Oral-facial-digital syndrome; Ornithine aminotransferase deficiency; Orofacial cleft 11 and 7, Cleft lip/palate-ectodermal dysplasia syndrome; Orstavik Lindemann Solberg syndrome; Osteoarthritis with mild chondrodysplasia; Osteochondritis dissecans; Osteogenesis imperfecta type 12, type 5, type 7, type 8, type I, type III, with normal sclerae, dominant form, recessive perinatal lethal; Osteopathia striata with cranial sclerosis; Osteopetrosis autosomal dominant type 1 and 2, recessive 4, recessive 1, recessive 6; Osteoporosis with pseudoglioma; Oto-palato-digital syndrome, types I and II; Ovarian dysgenesis 1; Ovarioleukodystrophy; Pachyonychia congenita 4 and type 2; Paget disease of bone, familial; Pallister-Hall syndrome; Palmoplantar keratoderma, nonepidermolytic, focal or diffuse; Pancreatic agenesis and congenital heart disease; Papillon-Lef\xc3\xa8vre syndrome; Paragangliomas 3; Paramyotonia congenita of von Eulenburg; Parathyroid carcinoma; Parkinson disease 14, 15, 19 (juvenile-onset), 2, 20 (early-onset), 6, (autosomal recessive early-onset, and 9; Partial albinism; Partial hypoxanthine-guanine phosphoribosyltransferase deficiency; Patterned dystrophy of retinal pigment epithelium; PC-K6a; Pelizaeus-Merzbacher disease; Pendred syndrome; Peripheral demyelinating neuropathy, central dysmyelination; Hirschsprung disease; Permanent neonatal diabetes mellitus; Diabetes mellitus, permanent neonatal, with neurologic features; Neonatal insulin-dependent diabetes mellitus; Maturity-onset diabetes of the young, type 2; Peroxisome biogenesis disorder 14B, 2A, 4A, 5B, 6A, 7A, and 7B; Perrault syndrome 4; Perry syndrome; Persistent hyperinsulinemic hypoglycemia of infancy; familial hyperinsulinism; Phenotypes; Phenylketonuria; Pheochromocytoma; Hereditary Paraganglioma-Pheochromocytoma Syndromes; Paragangliomas 1; Carcinoid tumor of intestine; Cowden syndrome 3; Phosphoglycerate dehydrogenase deficiency; Phosphoglycerate kinase 1 deficiency; Photosensitive trichothiodystrophy; Phytanic acid storage disease; Pick disease; Pierson syndrome; Pigmentary retinal dystrophy; Pigmented nodular adrenocortical disease, primary, 1; Pilomatrixoma; Pitt-Hopkins syndrome; Pituitary dependent hypercortisolism; Pituitary hormone deficiency, combined 1, 2, 3, and 4; Plasminogen activator inhibitor type 1 deficiency; Plasminogen deficiency, type I; Platelet-type bleeding disorder 15 and 8; Poikiloderma, hereditary fibrosing, with tendon contractures, myopathy, and pulmonary fibrosis; Polycystic kidney disease 2, adult type, and infantile type; Polycystic lipomembranous osteodysplasia with sclerosing leukoencephalopathy; Polyglucosan body myopathy 1 with or without immunodeficiency; Polymicrogyria, asymmetric, bilateral frontoparietal; Polyneuropathy, hearing loss, ataxia, retinitis pigmentosa, and cataract; Pontocerebellar hypoplasia type 4; Popliteal pterygium syndrome; Porencephaly 2; Porokeratosis 8, disseminated superficial actinic type; Porphobilinogen synthase deficiency; Porphyria cutanea tarda; Posterior column ataxia with retinitis pigmentosa; Posterior polar cataract type 2; Prader-Willi-like syndrome; Premature ovarian failure 4, 5, 7, and 9; Primary autosomal recessive microcephaly 10, 2, 3, and 5; Primary ciliary dyskinesia 24; Primary dilated cardiomyopathy; Left ventricular noncompaction 6; 4, Left ventricular noncompaction 10; Paroxysmal atrial fibrillation; Primary hyperoxaluria, type I, type, and type III; Primary hypertrophic osteoarthropathy, autosomal recessive 2; Primary hypomagnesemia; Primary open angle glaucoma juvenile onset 1; Primary pulmonary hypertension; Primrose syndrome; Progressive familial heart block type 1B; Progressive familial intrahepatic cholestasis 2 and 3; Progressive intrahepatic cholestasis; Progressive myoclonus epilepsy with ataxia; Progressive pseudorheumatoid dysplasia; Progressive sclerosing poliodystrophy; Prolidase deficiency; Proline dehydrogenase deficiency; Schizophrenia 4; Properdin deficiency, X-linked; Propionic academia; Proprotein convertase 1/3 deficiency; Prostate cancer, hereditary, 2; Protan defect; Proteinuria; Finnish congenital nephrotic syndrome; Proteus syndrome; Breast adenocarcinoma; Pseudoachondroplastic spondyloepiphyseal dysplasia syndrome; Pseudohypoaldosteronism type 1 autosomal dominant and recessive and type 2; Pseudohypoparathyroidism type 1A, Pseudopseudohypoparathyroidism; Pseudoneonatal adrenoleukodystrophy; Pseudoprimary hyperaldosteronism; Pseudoxanthoma elasticum; Generalized arterial calcification of infancy 2; Pseudoxanthoma elasticum-like disorder with multiple coagulation factor deficiency; Psoriasis susceptibility 2; PTEN hamartoma tumor syndrome; Pulmonary arterial hypertension related to hereditary hemorrhagic telangiectasia; Pulmonary Fibrosis And/Or Bone Marrow Failure, Telomere-Related, 1 and 3; Pulmonary hypertension, primary, 1, with hereditary hemorrhagic telangiectasia; Purine-nucleoside phosphorylase deficiency; Pyruvate carboxylase deficiency; Pyruvate dehydrogenase E1-alpha deficiency; Pyruvate kinase deficiency of red cells; Raine syndrome; Rasopathy; Recessive dystrophic epidermolysis bullosa; Nail disorder, nonsyndromic congenital, 8; Reifenstein syndrome; Renal adysplasia; Renal carnitine transport defect; Renal coloboma syndrome; Renal dysplasia; Renal dysplasia, retinal pigmentary dystrophy, cerebellar ataxia and skeletal dysplasia; Renal tubular acidosis, distal, autosomal recessive, with late-onset sensorineural hearing loss, or with hemolytic anemia; Renal tubular acidosis, proximal, with ocular abnormalities and mental retardation; Retinal cone dystrophy 3B; Retinitis pigmentosa; Retinitis pigmentosa 10, 11, 12, 14, 15, 17, and 19; Retinitis pigmentosa 2, 20, 25, 35, 36, 38, 39, 4, 40, 43, 45, 48, 66, 7, 70, 72; Retinoblastoma; Rett disorder; Rhabdoid tumor predisposition syndrome 2; Rhegmatogenous retinal detachment, autosomal dominant; Rhizomelic chondrodysplasia punctata type 2 and type 3; Roberts-SC phocomelia syndrome; Robinow Sorauf syndrome; Robinow syndrome, autosomal recessive, autosomal recessive, with brachy-syn-polydactyly; Rothmund-Thomson syndrome; Rapadilino syndrome; RRM2B-related mitochondrial disease; Rubinstein-Taybi syndrome; Salla disease; Sandhoff disease, adult and infantil types; Sarcoidosis, early-onset; Blau syndrome; Schindler disease, type 1; Schizencephaly; Schizophrenia 15; Schneckenbecken dysplasia; Schwannomatosis 2; Schwartz Jampel syndrome type 1; Sclerocornea, autosomal recessive; Sclerosteosis; Secondary hypothyroidism; Segawa syndrome, autosomal recessive; Senior-Loken syndrome 4 and 5, Sensory ataxic neuropathy, dysarthria, and ophthalmoparesis; Sepiapterin reductase deficiency; SeSAME syndrome; Severe combined immunodeficiency due to ADA deficiency, with microcephaly, growth retardation, and sensitivity to ionizing radiation, atypical, autosomal recessive, T cell-negative, B cell-positive, NK cell-negative of NK-positive; Partial adenosine deaminase deficiency; Severe congenital neutropenia; Severe congenital neutropenia 3, autosomal recessive or dominant; Severe congenital neutropenia and 6, autosomal recessive; Severe myoclonic epilepsy in infancy; Generalized epilepsy with febrile seizures plus, types 1 and 2; Severe X-linked myotubular myopathy; Short QT syndrome 3; Short stature with nonspecific skeletal abnormalities; Short stature, auditory canal atresia, mandibular hypoplasia, skeletal abnormalities; Short stature, onychodysplasia, facial dysmorphism, and hypotrichosis; Primordial dwarfism; Short-rib thoracic dysplasia 11 or 3 with or without polydactyly; Sialidosis type I and II; Silver spastic paraplegia syndrome; Slowed nerve conduction velocity, autosomal dominant; Smith-Lemli-Opitz syndrome; Snyder Robinson syndrome; Somatotroph adenoma; Prolactinoma; familial, Pituitary adenoma predisposition; Sotos syndrome 1 or 2; Spastic ataxia 5, autosomal recessive, Charlevoix-Saguenay type, 1, 10, or 11, autosomal recessive; Amyotrophic lateral sclerosis type 5; Spastic paraplegia 15, 2, 3, 35, 39, 4, autosomal dominant, 55, autosomal recessive, and 5A; Bile acid synthesis defect, congenital, 3; Spermatogenic failure 11, 3, and 8; Spherocytosis types 4 and 5; Spheroid body myopathy; Spinal muscular atrophy, lower extremity predominant 2, autosomal dominant; Spinal muscular atrophy, type II; Spinocerebellar ataxia 14, 21, 35, 40, and 6; Spinocerebellar ataxia autosomal recessive 1 and 16; Splenic hypoplasia; Spondylocarpotarsal synostosis syndrome; Spondylocheirodysplasia, Ehlers-Danlos syndrome-like, with immune dysregulation, Aggrecan type, with congenital joint dislocations, short limb-hand type, Sedaghatian type, with cone-rod dystrophy, and Kozlowski type; Parastremmatic dwarfism; Stargardt disease 1; Cone-rod dystrophy 3; Stickler syndrome type 1; Kniest dysplasia; Stickler syndrome, types 1 (nonsyndromic ocular) and 4; Sting-associated vasculopathy, infantile-onset; Stormorken syndrome; Sturge-Weber syndrome, Capillary malformations, congenital, 1; Succinyl-CoA acetoacetate transferase deficiency; Sucrase-isomaltase deficiency; Sudden infant death syndrome; Sulfite oxidase deficiency, isolated; Supravalvar aortic stenosis; Surfactant metabolism dysfunction, pulmonary, 2 and 3; Symphalangism, proximal, lb; Syndactyly Cenani Lenz type; Syndactyly type 3; Syndromic X-linked mental retardation 16; Talipes equinovarus; Tangier disease; TARP syndrome; Tay-Sachs disease, B1 variant, Gm2-gangliosidosis (adult), Gm2-gangliosidosis (adult-onset); Temtamy syndrome; Tenorio Syndrome; Terminal osseous dysplasia; Testosterone 17-beta-dehydrogenase deficiency; Tetraamelia, autosomal recessive; Tetralogy of Fallot; Hypoplastic left heart syndrome 2; Truncus arteriosus; Malformation of the heart and great vessels; Ventricular septal defect 1; Thiel-Behnke corneal dystrophy; Thoracic aortic aneurysms and aortic dissections; Marfanoid habitus; Three M syndrome 2; Thrombocytopenia, platelet dysfunction, hemolysis, and imbalanced globin synthesis; Thrombocytopenia, X-linked; Thrombophilia, hereditary, due to protein C deficiency, autosomal dominant and recessive; Thyroid agenesis; Thyroid cancer, follicular; Thyroid hormone metabolism, abnormal; Thyroid hormone resistance, generalized, autosomal dominant; Thyrotoxic periodic paralysis and Thyrotoxic periodic paralysis 2; Thyrotropin-releasing hormone resistance, generalized; Timothy syndrome; TNF receptor-associated periodic fever syndrome (TRAPS); Tooth agenesis, selective, 3 and 4; Torsades de pointes; Townes-Brocks-branchiootorenal-like syndrome; Transient bullous dermolysis of the newborn; Treacher collins syndrome 1; Trichomegaly with mental retardation, dwarfism and pigmentary degeneration of retina; Trichorhinophalangeal dysplasia type I; Trichorhinophalangeal syndrome type 3; Trimethylaminuria; Tuberous sclerosis syndrome; Lymphangiomyomatosis; Tuberous sclerosis 1 and 2; Tyrosinase-negative oculocutaneous albinism; Tyrosinase-positive oculocutaneous albinism; Tyrosinemia type I; UDPglucose-4-epimerase deficiency; Ullrich congenital muscular dystrophy; Ulna and fibula absence of with severe limb deficiency; Upshaw-Schulman syndrome; Urocanate hydratase deficiency; Usher syndrome, types 1, 1B, 1D, 1G, 2A, 2C, and 2D; Retinitis pigmentosa 39; UV-sensitive syndrome; Van der Woude syndrome; Van Maldergem syndrome 2; Hennekam lymphangiectasia-lymphedema syndrome 2; Variegate porphyria; Ventriculomegaly with cystic kidney disease; Verheij syndrome; Very long chain acyl-CoA dehydrogenase deficiency; Vesicoureteral reflux 8; Visceral heterotaxy 5, autosomal; Visceral myopathy; Vitamin D-dependent rickets, types land 2; Vitelliform dystrophy; von Willebrand disease type 2M and type 3; Waardenburg syndrome type 1, 4C, and 2E (with neurologic involvement); Klein-Waardenberg syndrome; Walker-Warburg congenital muscular dystrophy; Warburg micro syndrome 2 and 4; Warts, hypogammaglobulinemia, infections, and myelokathexis; Weaver syndrome; Weill-Marchesani syndrome 1 and 3; Weill-Marchesani-like syndrome; Weissenbacher-Zweymuller syndrome; Werdnig-Hoffmann disease; Charcot-Marie-Tooth disease; Werner syndrome; WFS1-Related Disorders; Wiedemann-Steiner syndrome; Wilson disease; Wolfram-like syndrome, autosomal dominant; Worth disease; Van Buchem disease type 2; Xeroderma pigmentosum, complementation group b, group D, group E, and group G; X-linked agammaglobulinemia; X-linked hereditary motor and sensory neuropathy; X-linked ichthyosis with steryl-sulfatase deficiency; X-linked periventricular heterotopia; Oto-palato-digital syndrome, type I; X-linked severe combined immunodeficiency; Zimmermann-Laband syndrome and Zimmermann-Laband syndrome 2; and Zonular pulverulent cataract 3.

In some aspects, the present disclosure provides uses of any one of the base editors described herein and a guide RNA targeting this base editor to a target A:T base pair in a nucleic acid molecule in the manufacture of a kit for nucleic acid editing, wherein the nucleic acid editing comprises contacting the nucleic acid molecule with the base editor and guide RNA under conditions suitable for the substitution of the adenine (A) of the A:T nucleobase pair with an guanine (G). In some embodiments of these uses, the nucleic acid molecule is a double-stranded DNA molecule. In some embodiments, the step of contacting induces separation of the double-stranded DNA at a target region. In some embodiments, the step of contacting thereby comprises the nicking of one strand of the double-stranded DNA, wherein the one strand comprises an unmutated strand that comprises the T of the target A:T nucleobase pair.

In some aspects, the present disclosure provides uses of any one of the base editors described herein and a guide RNA targeting this base editor to a target A:T base pair in a nucleic acid molecule in the manufacture of a kit for evaluating the off-target effects of a base editor, wherein the step of evaluating the off-target effects comprises contacting the base editor with the nucleic acid molecule and determining off-target effects in accordance with any one of the disclosed methods. In some embodiments of these uses, the nucleic acid molecule is a double-stranded DNA molecule. In some embodiments, the step of contacting of induces separation of the double-stranded DNA at a target region. In some embodiments, the step of contacting thereby comprises the nicking of one strand of the double-stranded DNA, wherein the one strand comprises an unmutated strand that comprises the T of the target A:T nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a cell, such as a human or non-human animal cell.

The present disclosure also provides uses of any one of the base editors described herein as a medicament. The present disclosure also provides uses of any one of the complexes of base editors and guide RNAs described herein as a medicament.

### Pharmaceutical Compositions

Other aspects of the present disclosure relate to pharmaceutical compositions comprising any of the adenosine deaminases, base editors, or the base editor-gRNA complexes described herein. Still other aspects of the present disclosure relate to pharmaceutical compositions comprising any of the polynucleotides or vectors that comprise a nucleic acid segment that encodes the adenosine deaminases, base editors, or the base editor-gRNA complexes described herein. The disclosure further provides pharmaceutical compositions that comprise particles comprising the rAAV vectors, dual rAAV vectors and ribonucleoproteins described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

In some embodiments, any of the base editors, gRNAs, and/or complexes described herein are provided as part of a pharmaceutical composition. In some embodiments, the pharmaceutical composition comprises any of the base editors provided herein. In some embodiments, the pharmaceutical composition comprises any of the complexes provided herein. In some embodiments pharmaceutical composition comprises a gRNA, a base editor, and a pharmaceutically acceptable excipient. Pharmaceutical compositions may optionally comprise one or more additional therapeutically active substances.

In some embodiments, compositions provided herein are formulated for delivery to a subject, for example, to a human subject, in order to effect a targeted genomic modification within the subject. In some embodiments, cells are obtained from the subject and contacted with a any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; 7,163,824, 9,526,784; 9,737,604; and U.S. Patent Publication Nos. 2018/0127780, published May 10, 2018, and 2018/0236081, published Aug. 23, 2018, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21′ Edition, A. R. Gennaro (Lippincott, Williams & Wilkins, Baltimore, Md., 2006; incorporated in its entirety herein by reference) discloses various excipients used in formulating pharmaceutical compositions and known techniques for the preparation thereof. See also PCT application PCT/US2010/055131, filed Nov. 2, 2010 (Publication No. WO 2011/053982, published May 5, 2011), incorporated in its entirety herein by reference, for additional suitable methods, reagents, excipients and solvents for producing pharmaceutical compositions comprising a nuclease. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials may be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition may be contained within a lipid particle or vesicle, such as a liposome or microcrystal, which is also suitable for parenteral administration. The particles may be of any suitable structure, such as unilamellar or plurilamellar, so long as compositions are contained therein. Compounds may be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition may be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent may be used for reconstitution or dilution of the lyophilized compound of the invention. Optionally associated with such container(s) may be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Delivery Methods

The disclosure also provides methods for delivering an adenine base editor described herein (e.g., in the form of an evolved base editor as described herein, or a vector or construct encoding same) into a cell. Such methods may involve transducing (e.g., via transfection) cells with a plurality of complexes each comprising a base editor and a gRNA molecule. In some embodiments, the gRNA is bound to the napDNAbp domain (e.g., nCas9 domain) of the base editor. In some embodiments, each gRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that is complementary to a target sequence. In certain embodiments, the methods involve the transfection of nucleic acid constructs (e.g., plasmids and mRNA constructs) that each (or together) encode the components of a complex of base editor and gRNA molecule. In certain embodiments, any of the disclosed base editors and a gRNA are administered as a protein:RNA complex, such as a ribonucleoprotein complex. In some embodiments, any of the disclosed base editors are administered as an mRNA construct, along with the gRNA molecule. In particular embodiments, administration to cells is achieved by electroporation or lipofection.

In certain embodiments of the disclosed methods, a nucleic acid construct (e.g., an mRNA construct) that encodes the base editor is transfected into the cell separately from the construct that encodes the gRNA molecule. In certain embodiments, these components are encoded on a single construct and transfected together. In other embodiments, the methods disclosed herein involve the introduction into cells of a complex comprising a base editor and gRNA molecule that has been expressed and cloned outside of these cells.

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell.

In some embodiments, the method of delivery provided comprises nucleofection, microinjection, biolistics, virosomes, liposomes, immunoliposomes, polycation or lipid:nucleic acid conjugates, naked DNA, artificial virions, and agent-enhanced uptake of DNA.

In another aspect, the disclosure discloses a pharmaceutical composition comprising any one of the presently disclosed vectors. In certain embodiments, the pharmaceutical composition further comprises a pharmaceutically acceptable excipient. In certain embodiments, the pharmaceutical composition further comprises a lipid and/or polymer. In certain embodiments, the lipid and/or polymer is cationic. The preparation of such lipid particles is well known. See, e.g. U.S. Pat. Nos. 4,880,635; 4,906,477; 4,911,928; 4,917,951; 4,920,016; 4,921,757; and 9,737,604, each of which is incorporated herein by reference.

Exemplary methods of delivery of nucleic acids include lipofection, nucleofection, electoporation (e.g., MaxCyte electroporation), stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 91/17424; WO 91/16024. Delivery may be to cells (e.g. in vitro or ex vivo administration) or target tissues (e.g. in vivo administration). Delivery may be achieved through the use of RNP complexes.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In other embodiments, the method of delivery and vector provided herein is an RNP complex. RNP delivery of base editors markedly increases the DNA specificity of base editing. RNP delivery of base editors leads to decoupling of on- and off-target DNA editing. RNP delivery ablates off-target editing at non-repetitive sites while maintaining on-target editing comparable to plasmid delivery, and greatly reduces off-target DNA editing even at the highly repetitive VEGFA site 2. See Rees, H. A. et al., Improving the DNA specificity and applicability of base editing through protein engineering and protein delivery, Nat. Commun. 8, 15790 (2017), U.S. Pat. No. 9,526,784, issued Dec. 27, 2016, and U.S. Pat. No. 9,737,604, issued Aug. 22, 2017, each of which is incorporated by reference herein.

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and w2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. Reference is made to US 2003/0087817, published May 8, 2003, International Patent Application No. WO 2016/205764, published Dec. 22, 2016, International Patent Application No. WO 2018/071868, published Apr. 19, 2018, U.S. Patent Publication No. 2018/0127780, published May 10, 2018, and International Publication No. WO2020/236982, published Nov. 26, 2020, the disclosures of each of which are incorporated herein by reference.

In various embodiments, the base editor constructs (including, the split-constructs) may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a whole or split base editor that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVrh.74, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AAV5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol. Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

In some embodiments, the base editors can be divided at a split site and provided as two halves of a whole/complete base editor. The two halves can be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning ABE.

These split intein-based methods overcome several barriers to in vivo delivery. For example, the DNA encoding base editors is larger than the recombinant AAV (rAAV) packaging limit, and so requires different solutions. One such solution is formulating the editor fused to split intein pairs that are packaged into two separate rAAV particles that, when co-delivered to a cell, reconstitute the functional editor protein. Several other special considerations to account for the unique features of base editing are described, including the optimization of second-site nicking targets and properly packaging base editors into virus vectors, including lentiviruses and rAAV.

Accordingly, the disclosure provides dual rAAV vectors and dual rAAV vector particles that comprise expression constructs that encode two halves of any of the disclosed base editors, wherein the encoded base editor is divided between the two halves at a split site. In some embodiments, the two halves may be delivered to cells (e.g., as expressed proteins or on separate expression vectors) and once in contact inside the cell, the two halves form the complete base editor through the self-splicing action of the inteins on each base editor half. Split intein sequences can be engineered into each of the halves of the encoded base editor to facilitate their transplicing inside the cell and the concomitant restoration of the complete, functioning ABE.

In various embodiments, the base editors may be engineered as two half proteins (i.e., an ABE N-terminal half and a ABE C-terminal half) by “splitting” the whole base editor as a “split site.” The “split site” refers to the location of insertion of split intein sequences (i.e., the N intein and the C intein) between two adjacent amino acid residues in the base editor. More specifically, the “split site” refers to the location of dividing the whole base editor into two separate halves, wherein in each halve is fused at the split site to either the N intein or the C intein motifs. The split site can be at any suitable location in the base editor, but preferably the split site is located at a position that allows for the formation of two half proteins which are appropriately sized for delivery (e.g., by expression vector) and wherein the inteins, which are fused to each half protein at the split site termini, are available to sufficiently interact with one another when one half protein contacts the other half protein inside the cell.

Additional methods for the delivery of nucleic acids to cells are known to those skilled in the art. See, for example, US Pub. No. 2003/0087817, incorporated herein by reference.

It should be appreciated that any base editor, e.g., any of the base editors provided herein, may be introduced into the cell in any suitable way, either stably or transiently. In some embodiments, a base editor may be transfected into the cell. In some embodiments, the cell may be transduced or transfected with a nucleic acid construct that encodes a base editor. For example, a cell may be transduced (e.g., with a virus encoding a base editor), or transfected (e.g., with a plasmid encoding a base editor) with a nucleic acid that encodes a base editor, or the translated base editor. Such transduction may be a stable or transient transduction. In some embodiments, cells expressing a base editor or containing a base editor may be transduced or transfected with one or more gRNA molecules, for example when the base editor comprises a Cas9 (e.g., nCas9) domain. In some embodiments, a plasmid expressing a base editor may be introduced into cells through electroporation, transient (e.g., lipofection) and stable genome integration (e.g., piggybac) and viral transduction or other methods known to those of skill in the art.

### Kits and Cells

Some aspects of this disclosure provide kits comprising a nucleic acid construct comprising a nucleotide sequence encoding an adenosine deaminase capable of deaminating an adenosine in a deoxyribonucleic acid (DNA) molecule. In some embodiments, the nucleotide sequence encodes any of the adenosine deaminases provided herein. In some embodiments, the nucleotide sequence comprises a heterologous promoter that drives expression of the adenosine deaminase. The nucleotide sequence may further comprise a heterologous promoter that drives expression of the gRNA, or a heterologous promoter that drives expression of the base editor and the gRNA.

In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone.

The disclosure further provides kits comprising a nucleic acid construct, comprising (a) a nucleotide sequence encoding a napDNAbp (e.g., a Cas9 domain) fused to an adenosine deaminase, or a base editor comprising a napDNAbp (e.g., Cas9 domain) and an adenosine deaminase as provided herein; and (b) a heterologous promoter that drives expression of the sequence of (a). In some embodiments, the kit further comprises an expression construct encoding a guide nucleic acid backbone, (e.g., a guide RNA backbone), wherein the construct comprises a cloning site positioned to allow the cloning of a nucleic acid sequence identical or complementary to a target sequence into the guide nucleic acid (e.g., guide RNA backbone).

Some embodiments of this disclosure provide cells comprising any of the base editors or complexes provided herein. In some embodiments, the cells comprise nucleotide constructs that encodes any of the base editors provided herein. In some embodiments, the cells comprise any of the nucleotides or vectors provided herein. In some embodiments, the cell is a stem cell. In some embodiments, the cell is a human stem cell, such as a human stem and progenitor cell (HSPC). In some embodiments, the cell is a mobilized (e.g., plerixafor-mobilized) peripheral blood HSPC.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. In some embodiments, the cell has been removed from a subject and contacted ex vivo with any of the disclosed base editors, complexes, vectors, or polynucleotides.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huhl, Huh4, Huh7, HUVEC, HASMC, HEKn, HEKa, MiaPaCell, Pancl, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalc1c7, HL-60, HMEC, HT-29, Jurkat, J Y cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some aspects, the present disclosure provides uses of any one of the base editors described herein and a guide RNA targeting this base editor to a target A:T base pair in a nucleic acid molecule in the manufacture of a kit for nucleic acid editing, wherein the nucleic acid editing comprises contacting the nucleic acid molecule with the base editor and guide RNA under conditions suitable for the substitution of the adenine (A) of the A:T nucleobase pair with an guanine (G). In some embodiments of these uses, the nucleic acid molecule is a double-stranded DNA molecule. In some embodiments, the step of contacting of induces separation of the double-stranded DNA at a target region. In some embodiments, the step of contacting thereby comprises nicking one strand of the double-stranded DNA, wherein the one strand comprises an unmutated strand that comprises the T of the target A:T nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a cell, such as a human or non-human animal cell.

The present disclosure also provides uses of any one of the adenine base editors described herein as a medicament. The present disclosure also provides uses of any one of the complexes of adenine base editors and guide RNAs described herein as a medicament.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### EXAMPLES

### Example 1

PACE is an ideal system for improving the kinetics of an enzyme because variant survival requires that gene III must be expressed before progeny phage are packaged, and before phage are diluted out of the lagoon³⁸ (see FIG. 1A). PACE is ideally suited to evolve a deoxyadenosine deaminase that can mediate deamination at a rate sufficient to enable efficient A•T-to-G•C base editing even when fused to Cas9 or Cas12 homologs that do not reside on DNA as long as SpCas9.

Following a previous development of a CBE PACE selection³⁸, a new PACE selection was developed that links ABE activity to expression of gene III on the AP (plasmid P1) (FIG. 1B and FIG. 18). ABE was divided into two components, each fused to half of a split intein³⁸. TadA-7.10 fused to a C-intein was encoded in the SP to focus mutagenesis and evolution on the TadA domain, and expressed catalytically dead Cas9 (dCas9) fused to an N-intein from a host-cell plasmid (P2) maintained in bacteria (FIG. 1B and FIG. 18)³⁸. Phage infection followed by intein trans-splicing generates full-length base editor protein, as was previously demonstrated during the development of PACE for CBEs³⁸. Although TadA functions natively as a dimer, the selections were performed for ABE activity using a single TadA-dCas9 fusion, as had done previously in E. coli, since it was presumed that the TadA-dCas9 fusion is able to dimerize either with itself or with endogenous E. coli TadA. It was envisioned that correcting one or more stop codons in a T7 RNA polymerase (T7 RNAP) gene on a third plasmid (P3) using ABE would thereby rescue T7 RNAP production to drive gene III expression from a T7 promoter (FIGS. 1B-1C and FIG. 18). Two stop codons at amino acid positions 57 and 58 in T7 RNAP were installed and provided a single guide RNA (sgRNA) that directs ABE to correct these stop codons on the transcription template strand back to Arg and Gln codons (FIGS. 1B-1C). Eight P3 variants of varying selection stringency were generated that use different promoters and ribosome binding site (RBS) strengths upstream of the T7 RNAP gene (FIG. 18), and then tested overnight propagation of SP encoding TadA-7.10 in host cells harboring P1, P2, and one of eight P3 variants (P3a-h). Phage propagation with host cells containing the least stringent P3 (P3a) was observed, as determined by measuring the number of plaque-forming units (PFU) before and after overnight incubation (FIG. 1D). These results suggest that P1+P2+P3a couples ABE activity to phage propagation, but the low rate of deamination of TadA-7.10 results in only modest gene III expression.

### Example 2

### TadA Evolution

Next, an attempt was made to evolve TadA-7.10 using this low-stringency phage propagation selection (P1+P2+P3a). Propagation was too weak to support PACE, resulting in phage washout. To overcome this, phage-assisted non-continuous evolution (PANCE)^{6, 29, 37, 39} was utilized.

PANCE of TadA-7.10 was initiated by infecting host cells harboring P1, P2, and either of the two least stringent P3 variants, P3a or P3b, with SP encoding TadA-7.10 in parallel evolutions (FIGS. 18-19). Genetic diversity was increased by allowing the TadA-7.10 to mutate in the absence of selection pressure during an initial period of “genetic drift”. Host cells were provided with a mutagenic drift plasmid (DP)^{13, 30, 40} that contained anhydrotetracycline (aTc)-inducible gene III. Host cells received aTc during passages 1, 3, and 7, allowing SP to freely diversify without any selection pressure (FIG. 19)³⁰. Phage propagation levels increased in later passages, suggesting that the population had accessed more active TadA variants. Selection stringency was increased by infecting the cells with fewer phage per passage (FIG. 19). After 15 passages on host cells harboring P3a and P3b, the phage pool showed increased activity, propagating overnight about 10,000-fold on host cells harboring P1, P2, and the four least stringent P3 variants (P3a, P3b, P3c, or P3d) and 100-1,000-fold with higher stringency P3 variants P3e and P3f (FIG. 1D). These findings suggest that PANCE with intermittent genetic drift enabled the initial evolution of TadA-7.10.

To characterize TadA-7.10 mutations that contributed to increased phage propagation during PANCE, 13 individual phage clones were isolated and their TadA-7.10 genes were sequenced. One mutation, T111R, was conserved in all 13 clones (FIG. 2A and FIG. 20). The mutation is predicted to lie near the active site of the enzyme, adjacent to D108N, a critical mutation that emerged early in the initial evolution of TadA-7.10 (FIG. 2B)². The prevalence and location of this mutation suggest that T111R contributes to increased ABE activity.

TadA-7.10 was continuously evolved from the phage pools that emerged from PANCE passage 15. During this second phase of PANCE experiments, four evolutions in parallel were performed, two on host cells harboring circuits P1, P2, and P3e, and two on host cells harboring P1, P2, and P3g (FIGS. 18-19). Both circuits are more stringent than those used during the initial PANCE experiments. Ten additional passages were completed (FIG. 19), then pooled the resulting phage and tested overnight phage propagation on host cells harboring P1, P2, and P3g. Phage following passage 25 demonstrated additional fitness increases, propagating overnight ˜100-fold on P1+P2+P3g host cells (FIG. 1D). 18 individual phage clones were isolated and their TadA genes were sequenced. PANCE using these more stringent circuits also enriched A109S or A109T (FIGS. 2A-2B and FIG. 20). Given the substantial increase in phage propagation strength after 25 total PANCE passages (surviving in total ˜10⁴⁹-fold dilution), it was decided to initiate PACE on the current phage population to benefit from its more stringent and efficient evolution system.

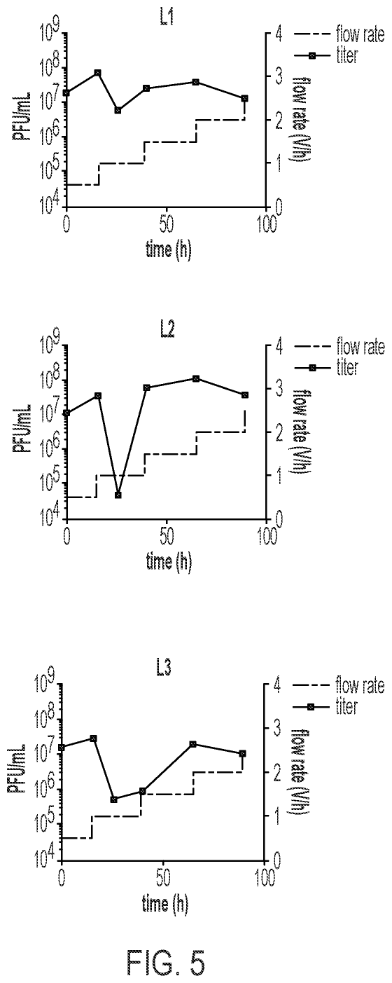

PACE was performed in host cells harboring P1, P2, and either P3e or P3g in parallel lagoons with phage pools from passage 25 of PANCE. The lagoon flow rate was gradually increased from 0.5 to 2.5 lagoon vol/h over 84 hours to require that T7 RNAP base editing occur quickly enough to support increasingly rapid phage propagation (FIG. 5). After 84 hours of PACE, an attempt to further increase the stringency by increasing the lagoon flow rate to 3.0 lagoon vol/h was made, but the phage could not propagate further (FIG. 5). Analysis of 21 individual SPs from 84 hours of PACE revealed enrichment of 11 consensus mutations across a variety of unique genotypes: R26G, V69A, V88A, A109S, T111R, D119N, H122N, Y147D, F149Y, T1661, D167N (FIGS. 2A-2B and FIG. 20). Phage emerging from PACE propagated for the first time on host cells harboring P1, P2, and P3h, which together form the most stringent circuit described herein (FIG. 1D). These observations suggest that the PANCE- and PACE-evolved TadA variants evolved greatly improved deoxyadenosine deamination activity.

### Comparison of Evolved TadA Variants

Five TadA genotypes were selected (TadA-8a, TadA-8b, TadA-8c, TadA-8d, and TadA-8e) emerging from PACE that collectively include all conserved mutations (FIG. 2A and FIG. 20) to characterize in mammalian cells. Unless otherwise noted, all mammalian cell experiments were performed using ABEs with optimized architecture, codon usage, and nuclear localization signals for ABEmax, which consists of the wild-type TadA monomer fused to the evolved TadA monomer, which was fused in turn to the Cas9 nickase domain (TadA-TadA*—Cas9 nickase, FIG. 2C)^{7, 16}. For clarity, hereafter ABEmax¹⁶ is referred to as ABE7.10. The resulting ABE8a-8e variants were tested for base editing activity in HEK293T cells along with ABE7.10¹⁶, which uses TadA-7.10 fused to SpCas9 nickase, and SaABEmax, which uses TadA-7.10 fused to SaCas9 nickase and has modest activity compared with ABEmax⁷. TadA-8a-e variants were also compared with TadA-7.10 for levels of A•T-to-G•C base editing when tethered to catalytically dead LbCas12a (dLbCas12a). Unlike SpCas9 and SaCas9, Cas12a variants that nick the non-edited strand have not been reported due to the lack of separate active sites that cleave each DNA strand in Cas12a⁴¹.

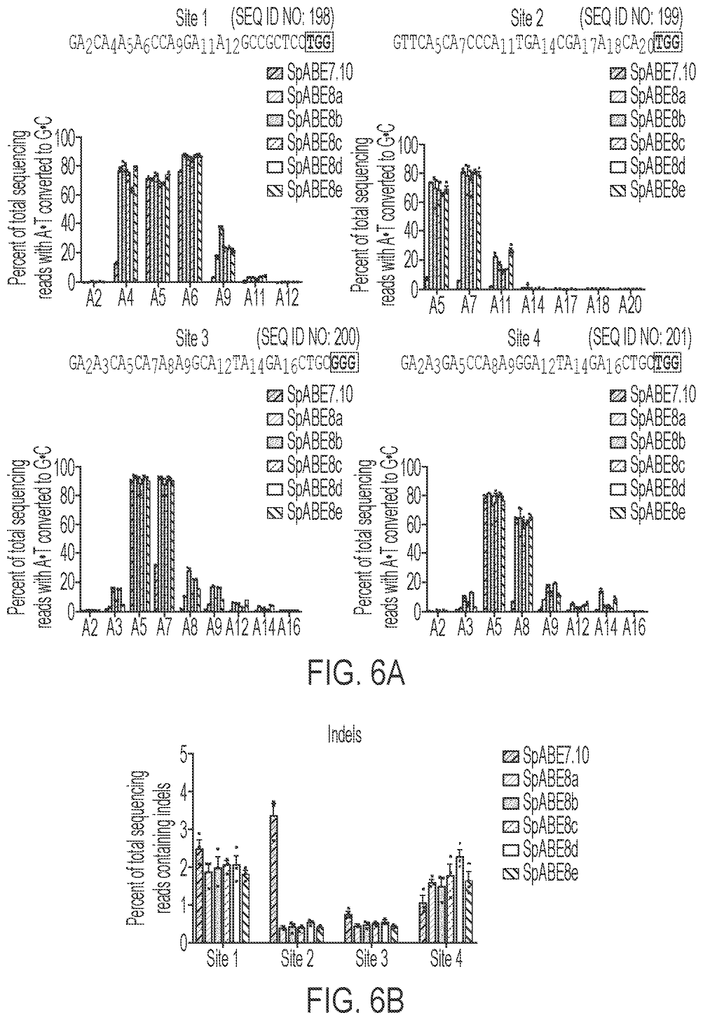

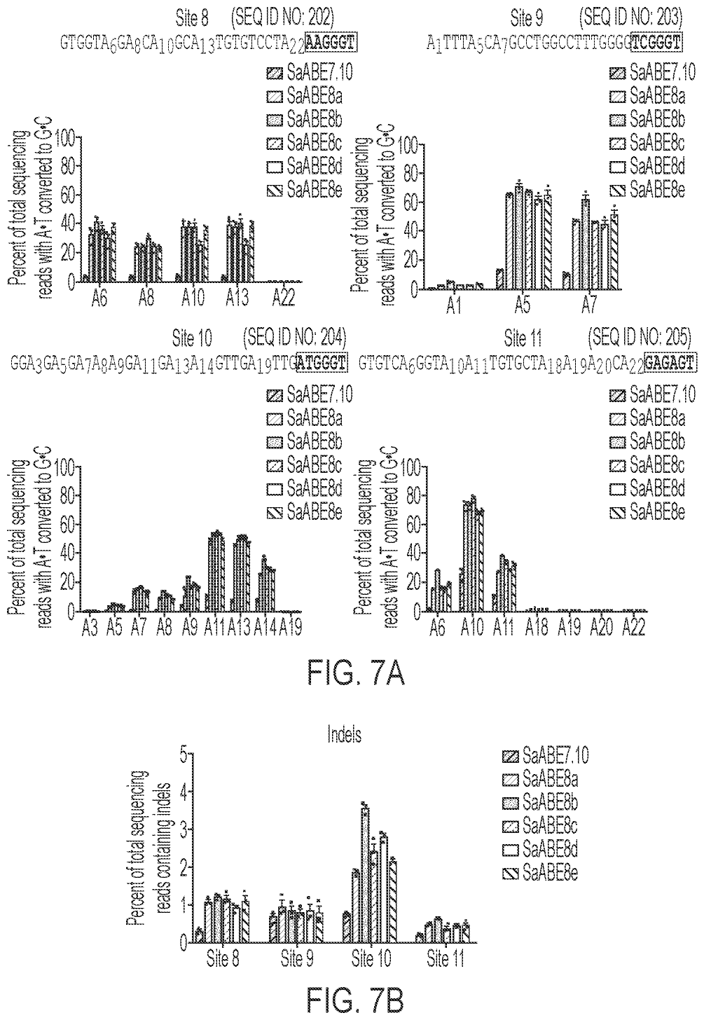

To select a TadA-8 variant for further studies, each base editor was transfected into HEK293T cells with a sgRNA targeting a site with a cognate PAM for SpCas9 (NGG), SaCas9 (NNGRRT) (SEQ ID NO: 488), or LbCas12a (TTTV) (SEQ ID NO: 489) and sequenced target loci after three days. Strikingly, all five TadA-8 variants demonstrated large improvements in A•T-to-G•C base editing efficiency, up to 9.4-fold, 12-fold, and 24-fold when tethered to SpCas9, SaCas9, and dLbCas12a, respectively, without significant changes to the very low indel formation levels of ABE7.10 (FIGS. 6A-8B). LbCas12a was used in the nuclease inactive form since the desired nickase enzyme for base editing has not been reported. Otherwise, all base editors were optimized with the architecture, codon usage, and nuclear localization signals of ABEmax¹⁶.

Most notably, efficient A•T-to-G•C base editing activity was observed with dLbCas12a for the first time, despite the lack of a suitable Cas12a nickase to nick the non-edited strand, which greatly increases base editing efficiency¹. Because ABEs containing TadA-8e showed consistently high editing activity and low indel formation, particularly when fused with dLbCas12a, base editors containing TadA-8e were chosen for subsequent analyses.

ABE variants were reported to exhibit comparable deamination activity with and without the wild-type TadA monomer in their architectures⁴². It was confirmed that eliminating the wild-type TadA from the wtTadA-TadA-8e-Cas fusion did not affect base editing activity with SpCas9, SaCas9, or dLbCas12a (FIGS. 9A-11B). In all subsequent analyses, ABEs were evaluated as simple TadA-8e-Cas variants, without the wild-type TadA monomer, hereafter referred to as ABE8e variants (FIG. 2C).

### Characterization of ABE8e

To determine if PANCE and PACE resulted in improved deamination kinetics, the DNA adenosine deamination kinetics of ABE7.10 and ABE8e were compared in vitro. Recombinant ABE7.10 and ABE8e was purified through His-tag column chromatography, high-resolution heparin-affinity chromatography, and size-exclusion chromatography. Single-turnover DNA deamination assays were performed by measuring A to I conversion in vitro to determine their apparent deamination rate constants (kₐₚₚ). Remarkably, kₐₚₚ of deoxyadenosine deamination is 590-fold higher for ABE8e than for ABE7.10 (0.59±0.034 min⁻¹ for ABE8e versus 0.0010±0.00030 min⁻¹ for ABE7.10) (FIG. 2D). The dramatically increased rate of deoxyadenosine deamination by ABE8e compared to ABE7.10 suggests that TadA-8e may be fast enough to yield efficient DNA adenosine deamination even when coupled to non-SpCas9 Cas effectors that have residence times on DNA substrates. These results establish that PANCE and PACE of TadA-7.10 resulted in a highly evolved TadA variant that mediates very efficient and fast deoxyadenosine deamination.

To begin to test the possibility that evolved TadA-8e may expand the targeting capabilities of adenine base editing, HEK293T cells were treated with SpABE8e, SaABE8e, LbABE8e, enAsABE8e, or the corresponding ABE7.10 variants targeting several endogenous genomic sites each and measured A:T-to-G:C base conversion efficiency after three days (FIG. 2E and FIGS. 9A-11B). With all four Cas homologs, substantially enhanced editing with the ABE8e variant compared to the ABE7.10 variant was observed. For SpABE variants, the increased editing efficiency of SpABE8e editors was most evident when examining the editing levels at the second-most efficiently edited A within each protospacer. With SpABE7.10, editing levels at the second-most edited A ranged from 1.7% to 20%, while with SpABE8e editing levels ranged from 18% to 86%, improvements of 3.0- to 11-fold.

ABE8e variants based on SaCas9 and on two Cas12a homologs showed large increases in editing levels. For SaABE7.10, A:T-to-G:C conversion at the highest editing position ranged from 3.6±1.1% to 26±5.1% at the five genomic loci tested, while for SaABE8e, A•T-to-G•C conversion at the highest editing position ranged from 33±5.8% to 69±11% (FIG. 2E). For Cas12a homologs, no nickase for the unedited strand is known. Therefore, base editors with dead LbCas12a and enAsCas12a (an engineered AsCas12a) were analyzed. Strikingly, average LbCas12a-mediated adenine base editing increased from 2.9% average editing for LbABE7.10 to 24% average editing (and as high as 46±5.0%) for LbABE8e, and average enAsCas12a-mediated editing increased 59-fold from 0.31% with enAsABE7.10 to 18% (and as high as 29±0.51%) for enAsABE8e at the mostly highly edited position within each protospacer (FIG. 2E)⁹.

Next, the base editing window of ABE8e variants was examined. The editing windows for SpCas9-derived ABE7.10 are slightly narrower (typically, protospacer positions 4-7, counting the PAM as positions 21-23) than the editing windows for canonical SpCas9 CBEs³⁸. Editing windows of ABE8e variants are now consistent with those of the corresponding CBEs: positions 4-8 for SpABE8e, 3-14 for SaABE8e, and 8-14 for both LbABE8e and enAsABE8e (FIGS. 9A-11B). These results collectively demonstrate that ABE8e variants can efficiently edit positions that were previously challenging to target with ABE7.10.

### Example 3

To further expand the targeting scope of ABEs caused by modest compatibility with alternative PAM Cas9 variants, ABE8e variants with Cas9 variants previously engineered or evolved to be compatible with alternative PAM sequences were generated, including SpCas9-NG (PAM=NGN)¹¹ (SEQ ID NO: 490) and SaCas9-KKH (PAM=NNNRRT)⁷ (SEQ ID NO: 491). Each variant was tested in HEK293T cells at five or more endogenous genomic sites each and measured A•T-to-G•C conversion after three days (FIG. 2F, FIGS. 10A-10B, and FIGS. 12A-13B). Compared to the corresponding ABE7.10 variants, NG-ABE8e and SaKKH-ABE8e demonstrated increased editing efficiencies (FIG. 2F, FIGS. 10A-10B, and FIGS. 12A-13B). For NG-ABEs, the 11 most highly edited adenines across five genomic sites were analyzed. While A•T-to-G•C conversion levels were similar between NG-ABE7.10 and NG-ABE8e at four of the 11 target adenines, at the other seven target adenines, NG-ABE8e was substantially (up to 28-fold) more efficient (FIG. 2F). Similarly, SaKKH-ABE8e supported increased editing efficiencies (37-78% at the most highly edited position) at all tested adenines at each of the nine loci tested compared with SaKKH-ABE7.10 (12-45% at the most highly edited position) (FIG. 2F). These data collectively show that TadA-8e is broadly compatible with diverse Cas9 and Cas12 homologs, greatly expanding the targeting scope of adenine base editors.

The use of circularly permuted Cas9 variants in base editors alters the position of base editing activity windows. It has been previously demonstrated that the editing windows of ABEs using circularly permuted Cas9 variants are broadened from protospacer positions 4-7 for canonical ABE7.10 to positions 4-11 for the circularly permuted variants. Circularly permuted CP1028-ABE8e and CP1041-ABE8e were constructed, and observed further expansion of the editing window to protospacer positions 3-14 (FIG. 2G and FIGS. 9A-9B) was observed. Increased editing was also observed at the boundaries of the editing window. From protospacer positions 9-14, which lie outside the canonical editing window of ABEs, base editing efficiency across seven tested genomic loci increased an average of 2.9- and 2.5-fold with CP1028-ABE8e and CP1041-ABE8e, respectively, compared to the corresponding ABE7.10 variants, without significant changes in indel frequencies (FIG. 2G and FIGS. 9A-9B). Together, circularly permuted ABE8e variants and non-canonical PAM ABE8e variants expand the targeting scope of efficient adenine base editing.

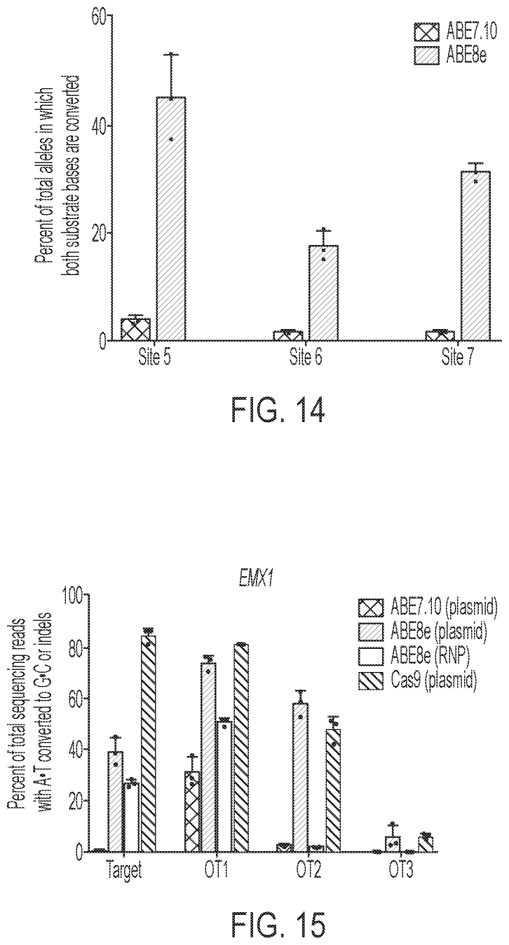

Next, an understanding of the differences in the processivity between ABE8e and ABE7.10 was sought. Enhanced processivity is useful for some applications such as the disruption of regulatory regions or genetic screens driven by base editing. The processivity of ABEs was assessed at genomic sites 5, 6, and 7 by calculating the number of alleles containing multiple A•T to G•C edits within the editing window. At these three sites, the frequency of alleles with multiple base conversions increase 11- to 17-fold for ABE8e relative to ABE7.10, respectively. (FIG. 14). These data suggest that ABE8e is particularly useful when multiple A•T-to-G•C conversions at a single locus are desired.

### Off-Target Analysis of ABE8e

Off-target activity of ABE8e in HEK293T cells was analyzed at known off-target sites previously reported for either ABEs or for Cas9 nuclease^{43, 44}. The top three known ABE off-target sites were tested for site 5 (HBG) and site 6 (VEGFA3) as identified by EndoV-Seq⁴³, and for EMX1 as identified by GUIDE-seq⁴⁴. An increase in editing was observed at six of the nine off-target sites when comparing ABE8e to ABE7.10, but the ratio of on-target to off-target editing was comparable at all but two sites (FIG. 3A). At the top three EMX1 off-targets sites⁴⁴, similar levels of editing were observed for ABE8e (averaging 74±3.0%, 58±4.9%, and 5.9±4.8% at the top three off-target sites, respectively) and Cas9 nuclease (averaging 81±2.2%, 48±4.9%, and 6.0±1.0%, respectively) (FIG. 15). Given the previous observations that off-target base editing is greatly reduced when delivering the base editor as protein-RNA (RNP) complexes⁴⁵, both on-target and off-target base editing with ABE8e RNP delivery were examined. RNP delivery of ABE8e purified from E. coli resulted in similar levels of on-target editing at, but much lower editing levels at the nine off-target sites, and thus a dramatic increase in on-target:off-target editing ratios (up to 1,300-fold) at every off-target site analyzed (FIG. 3A). A 1,300-fold increase in on-target:off-target editing ratios was observed at the EMX1 target, and a roughly 90-fold increase in on-target:off-target editing ratios was observed at the clinically relevant HBG target. Thus, for applications in which off-target editing must be minimized, the use of ABE8e RNP delivery is recommended.

ABE7.10 has previously been shown to cause a low-level increase in deamination of adenines in cellular RNA, which can be minimized by introducing mutations in the TadA domains that decrease RNA editing but preserve DNA editing^{42, 46-48} To measure the extent of cellular RNA editing by ABE8e, HEK293T cells were treated with plasmids encoding ABE7.10, ABE8e, or Cas9 (D10A) nickase and a guide RNA targeting LDLR, then measured the A-to-I mutation frequency across the entire transcriptome. Consistent with previous reports^{42, 46, 47}, a modest increase in transcriptome-wide RNA editing was detected with ABE7.10 compared to the Cas9 (D10A) control, with A-to-I conversions increasing from 0.19±0.017% for Cas9 (D10A) to 0.24±0.0066% for ABE7.10. ABE8e induced additional transcriptome-wide A-to-I conversion to 0.35±0.010%. To reduce RNA editing by ABE8e, mutations were installed that had been previously reported to minimize RNA editing of ABEs^{42, 47}, and transcriptome-wide A-to-I RNA editing levels were measured. Three ABE8e mutants were tested: ABE8e(TadA-8e V106W)⁴⁷, ABE8e(TadA-8e V82G)⁴², and ABE8e(TadA-8e K20A R21A)⁴². ABE8e(TadA-8e V106W) resulted in the greatest reduction in RNA editing levels, decreasing the transcriptome-wide A-to-I conversion level to 0.25±0.011% (FIG. 3B). On-target editing at the most efficiently edited protospacer position was similar among all of the tested mutants to that of ABE8e (FIG. 3B). The use of ABE8e(TadA-8e V106W) for applications that require minimizing off-target RNA editing is recommended.

Next, the ability of ABE8e to mediate Cas9-independent off-target DNA editing was characterized. A recently developed orthogonal R-loop assay⁵¹⁴ was used to detect the propensity of base editors to edit single-stranded DNA regions unrelated to their target loci with much greater sensitivity and lower cost than assays that require whole-genome sequencing (FIG. 3D). HEK293T cells were co-transfected with plasmids encoding an SpABE variant and an on-target sgRNA for SpABE, along with a catalytically inactive SaCas9 (dSaCas9) and an SaCas9 sgRNA targeting a genomic locus unrelated to the SpABE on-target site. Base editing levels within these dSaCas9-generated R-loops were previously correlated with the propensity for Cas9-independent off-target editing in mammalian cells⁵¹.

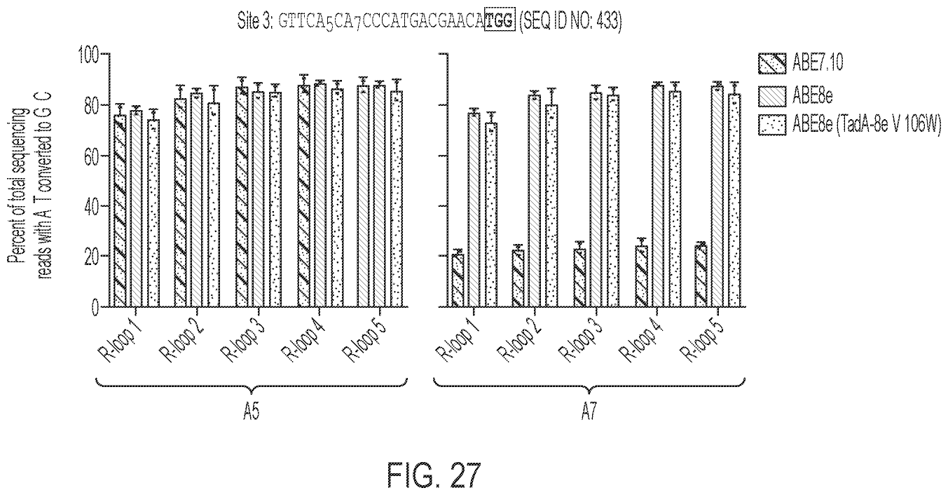

Using this assay, A•T-to-G•C base conversion levels were compared in five dSaCas9 R-loops for ABE7.10 and ABE8e, observing an increase in off-target editing at these orthogonal R-loops, ranging from 0.079%-0.32% with ABE7.10 to 1.9%-6.7% with ABE8e (FIG. 3E, FIG. 27). To reduce Cas9-independent off-target DNA editing, we performed the same analysis with ABE8e (TadA-8e V106W), resulting in a substantial decrease in off-target editing at these orthogonal R-loops of 0.32%-1.3% (FIG. 3E, FIG. 27). These data indicate that Cas9-independent off-target editing increases with ABE8e as expected given its higher activity, but can be ameliorated by introducing the V106W mutation into TadA-8e, which likely reduces affinity of TadA-8e for both DNA and RNA, thereby increasing its dependence on Cas9 for substrate engagement.

### Example 4: Disease Application of ABE8e Base Editors: HBG and BLCL11A Editing

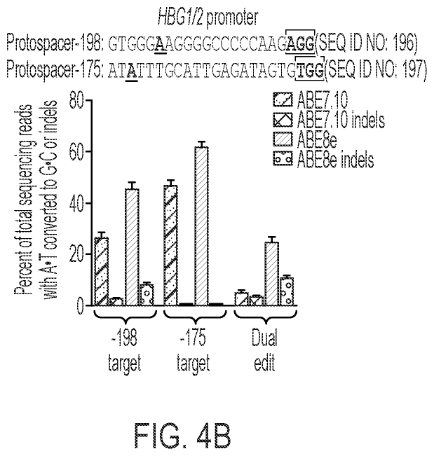

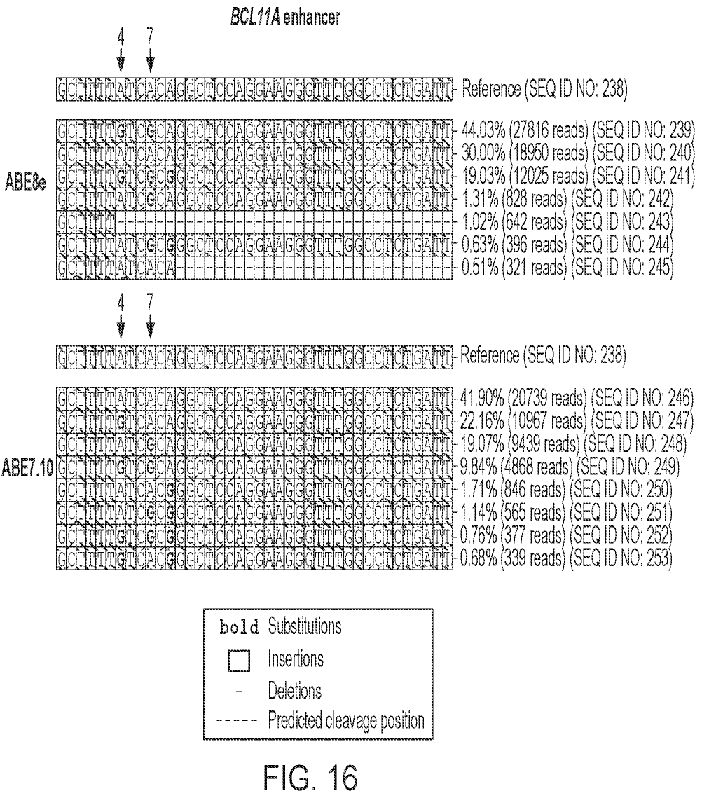

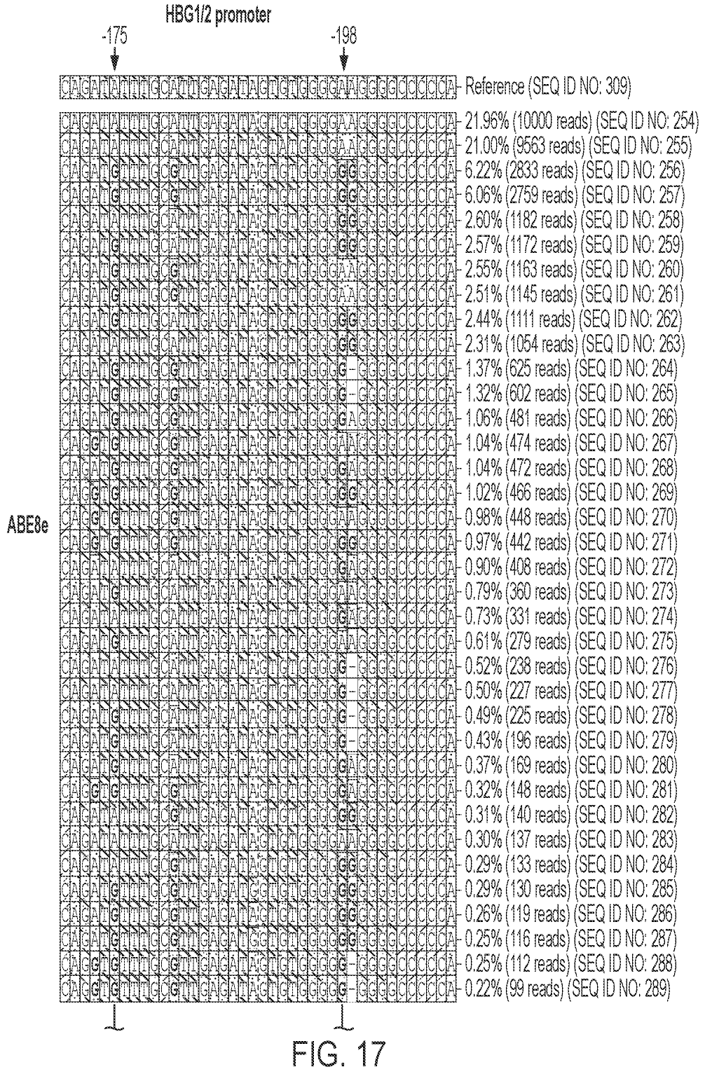

Two sites were targeted to install mutations known to induce the upregulation of fetal hemoglobin, a promising approach to treat hemoglobinopathies such as sickle cell disease and β-thalassemia⁴⁹. First, ABE8e editing was assessed at an erythroid enhancer of BCL11A, which encodes a transcriptional repressor that silences fetal hemoglobin in adult erythroid cells. Mutations in a GATA1 binding site at the +58 BCL11A erythroid enhancer lead to decreased BCL11A expression and fetal hemoglobin induction⁵⁰⁻⁵². The ability of ABE7.10 and ABE8e to install two A•T-to-G•C edits in the GATA1 binding site at positions 4 and 7 within a single protospacer in HEK293T cells was compared. Simultaneous editing of both target adenines was observed in 54.4±12.5% of alleles following cellular treatment with ABE8e compared to only 7.9±3.4% of alleles following treatment with SpABE7.10 (FIG. 4A and FIG. 16).

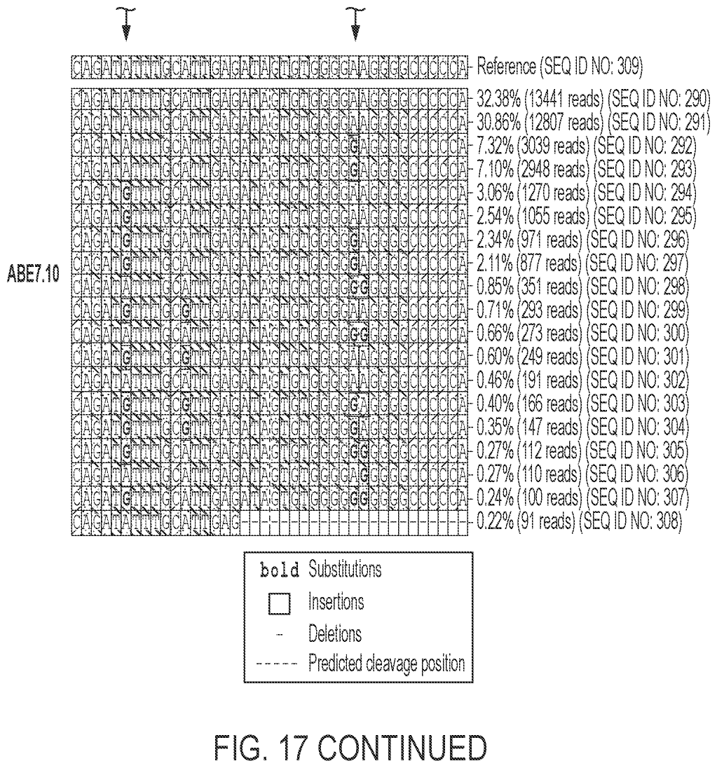

ABE8e was also applied to install two naturally-occurring A•T-to-G•C single-nucleotide polymorphisms (SNPs) present in the promoters of the HBG1 and HBG2 fetal hemoglobin genes⁴⁹. Each of these SNPs confer a hereditary persistence of fetal hemoglobin phenotype, which may alleviate symptoms of hemoglobinopathies⁵³. The target adenines are located at HBG promoter positions −198 and −175, and their base editing requires two guide RNAs since they are separated by 23 nucleotides. HEK293T cells were treated with ABE7.10 or ABE8e and each guide RNA separately, and moderate increases in editing at both sites with ABE8e were observed (FIG. 4B). Although each SNP individually increases the expression of fetal hemoglobin, editing both SNPs simultaneously might further augment fetal hemoglobin levels. When both guide RNAs were delivered simultaneously with ABE7.10, combined editing at both HBG promoter positions of only 5.1±0.9% within a single allele was observed (FIG. 4B and FIG. 17). In contrast, 24±2.2% of HBG alleles containing both edits with ABE8e were observed, a 4.8-fold increase (FIG. 4B and FIG. 17).

An increased percentage of alleles simultaneously containing A:T-to-G:C editing at positions −195 and −175 was observed after treatment with ABE8e compared to ABE7.10. Multiple edits are predicted to more effectively lead to the expression of fetal hemoglobin⁴⁹. These results suggest that ABE8e is especially well suited for multiplexed base editing applications.

### Discussion

Collectively, these results demonstrate the ability of PANCE and PACE to evolve an adenine base editor with 590-fold improved deamination kinetics that support efficient base editing using a much larger set of Cas9 and Cas12a homologs than was previously possible. The newly evolved TadA-8e supports efficient adenine base editing when fused with every Cas protein tested—SpCas9, SaCas9, LbCas12a, enAsCas12a, SpCas9-NG, SaCas9-KKH, SpCas9-CP1028, SpCas9-CP1041, and SpCas9-NG-CP1041—to mediate efficient A•T-to-G•C base conversions. The improved kinetics, editing efficiency, and targeting scope of ABE8e variants enabled efficient simultaneous base editing to install two mutations in a GATA1 binding site of a BCL11A enhancer, or two mutations in the promoter of HBG genes, all of which are known to increase fetal hemoglobin expression. ABE8e substantially advances the capabilities of adenine base editors by expanding their targeting scope, efficiency, and suitability for demanding base editing applications.

### Methods

### General Methods and Molecular Cloning

Antibiotics were used at the following working concentrations: carbenicillin, 50 μg/mL; spectinomycin, 50 μg/mL; chloramphenicol, 40 μg/mL; and kanamycin, 30 μg/mL. Nuclease-free water (ThermoFisher Scientific) was used for PCR reactions and cloning. For all other experiments, water was purified using a MilliQ purification system (Millipore). Phusion U Green Multiplex PCR Master Mix (ThermoFisher Scientifc) was used for all PCRs.

Plasmids were cloned by uracil-specific excision reagent (USER) assembly, Golden Gate assembly, or KLD cloning following manufacturer's instructions. For USER cloning, 42-60° C. melt temperature junctions were used, and constructs were assembled by digesting at 37° C. for 45 minutes followed by transformation into chemically competent cells. For Golden Gate assembly, Lgul (Sapl isoschizomer, Life Technologies) was used as the type IIS restriction enzymes along with T4 DNA ligase (New England BioLabs). Typical assemblies contained final concentrations of ˜0.5-2 ng per kb per μL plasmids, with a ˜2:1 ratio of donor to acceptor plasmids. Assemblies were incubated at 37° C. for between 1 hour and 18 hours followed by transformation into chemically competent cells. Guide RNA plasmids were assembled following the manufacturer's instructions with KLD enzyme mix (New England BioLabs).

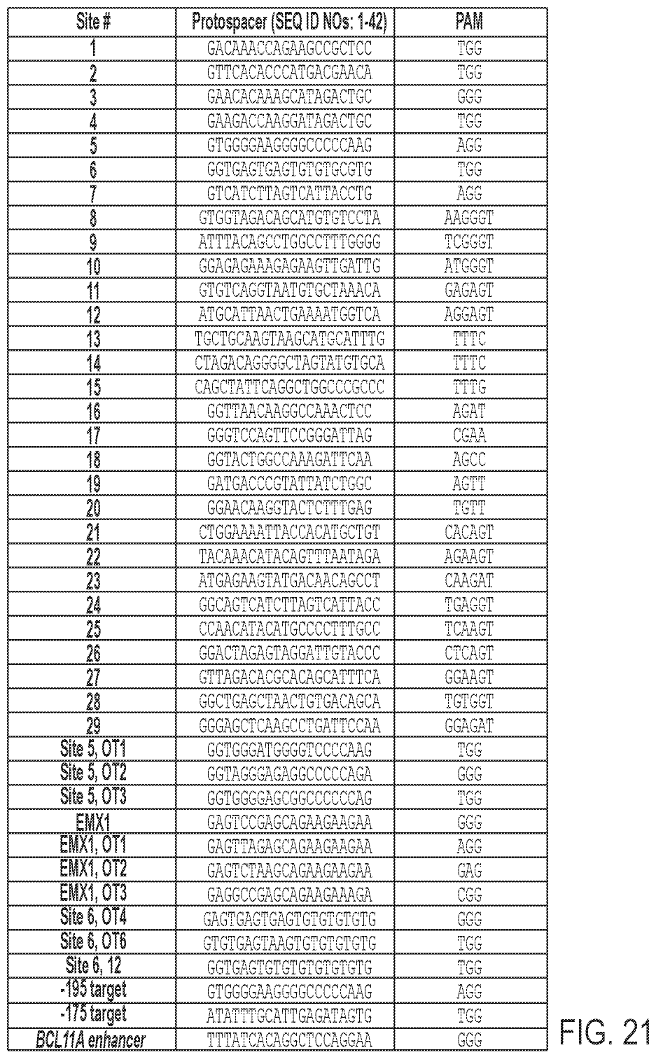

Codon-optimized sequences for human cell expression were obtained from Genscript. Plasmids were cloned and amplified using Mach1 T1^{R} competent cells (ThermoFisher Scientific). Plasmid DNA was isolated using the Qiagen Spin Miniprep Kit and Qiagen Midiprep Kit according to the manufacturer's instructions. All constructs assembled using PCR were fully sequence-verified using Sanger sequencing (Quintara Biosciences), while constructs assembled using Golden Gate cloning were sequence-verified across all assembly junctions. Protospacer sequences for guide RNA plasmids are described in FIG. 21. The amino acid sequences for codon-optimized, bis-bpNLS base editor variants are listed in Sequence section below.

### Preparation and Transformation of Chemically Competent Cells

Strain S2060³³ was used in all experiments, including phage propagation tests, PANCE, and PACE. Chemically competent cells were prepared as described³⁸, unless otherwise noted. Briefly, an overnight culture was diluted 50-fold into 2×YT media and grown at 37° C. with shaking at 230 r.p.m. to an optical density (0D600) of around 0.4-0.5. Cells were cooled on ice and pelleted by centrifugation at 4,000 g for 10 minutes at 4° C. The cell pellet was then resuspended by gentle stirring in ice-cold TSS solution (LB media supplemented with 5% v/v DMSO, 10% w/v PEG 3350, and 20 mM MgCl2). The cell suspension was mixed thoroughly, aliquoted and frozen in a dry ice/acetone bath, then stored at −80° C. until use. To transform cells, 100 μl of competent cells thawed on ice was added to a plasmid(s) and 100 μl KCM solution (100 mM KCl, 30 mM CaCl2, and 50 mM MgCl2 in water). The mixture was heat shocked at 42° C. for 60 seconds and SOC media (200 μL) was added. Cells were allowed to recover at 37° C. with shaking at 230 r.p.m. for 1 hour, then spread on LB media with 1.5% agar (United States Biologicals) plates containing the appropriate antibiotic(s) and incubated at 37° C. for 16-18 hours.

### Plaque Assays for Phage Titer Quantification

Phage were plagued on S2060³³ E. coli host cells containing plasmid pJC175e (activity-independent propagation)¹² or plasmid pT7-AP13 (to check for the presence of T7 RNAP recombinants)¹². To prepare a cell stock for plaquing, overnight culture of host cells (fresh or stored at 4° C. for up to −1 week) was diluted 50-fold in 2×YT media containing appropriate antibiotic(s) and grown at 37° C. to an OD₆₀₀ of 0.5-0.8. Serial dilutions of phage (ten-fold) were made in PBS buffer (pH 7.4) or water. To prepare plates, molten 2×YT medium agar (1.5% agar, 55° C.) was mixed with Bluo-gal (10% w/v in DMSO) to a final concentration of 0.04% Bluo-gal. The molten agar mixture was pipetted into quadrants of quartered Petri dishes (1.5 mL per quadrant) or wells of a 12-well plate (˜1 mL per well) and allowed to set. To prepare top agar, a 2:1 mixture of 2×YT media and molten 2×YT medium agar (1.5%, 0.5% agar final) was prepared. Top agar was maintained tightly capped at 55° C. for up to 1 week. To plaque, cell stock (50-100 μL) and phage (10 μL) were mixed in 2 mL library tubes (VWR International), and 55° C. top agar added (400 or 1,000 μL for 12-well plate or Petri dish, respectively) and mixed one time by pipetting up and down, and then the mixture was immediately pipetted onto the solid agar medium in one well of a 12-well plate or one quadrant of a quartered Petri dish. Top agar was allowed to set undisturbed (10 minutes at room temperature), then plates or dishes were incubated (without inverting) at 37° C. overnight. Phage titer were determined by quantifying blue plaques.

### Phage Propagation Assays

S2060 cells containing plasmids of interest were prepared as described above and inoculated in Davis Rich Medium (DRM) (prepared from US Biological CS050H-001/CS050H-003). Host cells from an overnight culture in DRM were diluted 50-fold into fresh DRM and grown for ˜1.5 hours at 37° C. Previously titered phage stocks were added to 2 mL of bacterial culture at a final concentration of 105 plaque forming units mL⁻¹. The cultures were grown overnight with shaking at 37° C. and then centrifuged (3,600 xg, 10 minutes) to remove cells. The supernatants were titered by plaquing as described above. Fold enrichment was calculated by dividing the titer of phage propagated on host cells by the titer of phage at the same input concentration shaken overnight in DRM without host cells.

### PANCE Experiments

Chemically competent host cells were transformed with DP6⁴⁰ and plated on 2×YT agar containing 0.5% glucose (w/v) along with appropriate concentrations of antibiotics. Five colonies were diluted in DRM with the appropriate antibiotics, grown to OD₆₀₀ 0.5-0.6, and treated with 40 mM arabinose to induce mutagenesis and the desired amount of anhydrotetracycline for a given passage (0 or 40 ng/mL). Treated cultures were split into the desired number of either 2 mL cultures in single culture tubes or 500 μL cultures in a 96-well plate and infected with selection phage. Infected cultures were grown overnight at 37° C. and harvested the next day via centrifugation (3000 g for 10 minutes). Supernatant containing evolved phage was isolated and stored at 4° C. Isolated phage were then used to infect the next passage and the process repeated for the desired number of selection passages for the selection. For phage dilutions, see FIG. 19. Phage titers were determined by plaquing as described above. Phage genotypes were assessed from pool samples or single plaques by diagnostic PCR using primers BT-52F (5′-GTCGGCGCAACTATCGGTATCAAGCTG (SEQ ID NO: 310)) and BT-52R2 (5′-AGTAAGCAGATAGCCGA ACAAAGTTACCAGAAGGAAAC (SEQ ID NO: 311)), and the PCR products were assessed by Sanger sequencing.

### PACE Experiments

Unless otherwise noted, PACE apparatus, including lagoons, chemostats, pumps and media, were prepared and used as previously described³⁹. Host cells were prepared as described for PANCE above. Five colonies were diluted into 5 mL DRM with the appropriate antibiotics and grown to OD₆₀₀ 0.4-0.8, which was then used to inoculate a chemostat (60 mL), which was maintained under continuous dilution with fresh DRM at 1-1.5 volumes per hour to keep cell density roughly constant. Lagoons were initially filled with DRM, then continuously diluted with chemostat culture for at least 2 hours before seeding with phage.

Stock solution of arabinose (1 M) was pumped directly into lagoons (10 mM final) as previously described³⁹ for 1 hour before the addition of phage. For the first 12 hours after phage inoculation, anhydrotetracycline (aTc) was present in the stock solution (3.3 μg/mL). Syringes containing aTc solution were covered in aluminum foil, and work was conducted to minimize light exposure of tubing and lagoons.

Lagoons were seeded at a starting titer of ˜10⁷ pfu per mL. Dilution rate was adjusted by modulating lagoon volume (5-20 mL) and/or culture inflow rate (10-20 mL/h). Lagoons were sampled at indicated times (usually every 24 hours) by removal of culture (500 μL) by syringe through the waste needle. Samples were centrifuged at 13,500 g for 2 minutes and the supernatant removed and stored at 4° C. Titers were evaluated by plaquing as described above. The presence of T7 RNAP or gene III recombinant phage was monitored by plaquing on 52060 cells containing pT7-AP and no plasmid. Phage genotypes were assessed from single plaques by diagnostic PCR as described in the PANCE section.

### Cell Culture

HEK293T cells (ATCC CRL-3216) were cultured in Dulbecco's modified Eagle's medium (Corning) supplemented with 10% fetal bovine serum (ThermoFisher Scientific) and maintained at 37° C. with 5% CO₂.

### Transfections

HEK293T cells were seeded at 50,000 cells per well on 48-well poly-D-lysine plates (Corning) in the same culture medium. Cells were transfected 24-30 hours after plating with 1.5 μL Lipofectamine 2000 (ThermoFisher Scientific) using 750 ng base editor plasmid, 250 ng guide RNA plasmid and 20 ng green fluorescent protein as a transfection control following the manufacturer's instructions. When targeting HBG1/2 and BCL11A sites with plasmid DNA, 150 ng of base editor plasmid and 50 ng of guide RNA plasmid was complexed with 1 μL of Lipofectamine 3000. Cells were cultured for 3 days, then washed with 1× PBS (ThermoFisher Scientific), followed by genomic DNA extraction by addition of 100 μL freshly prepared lysis buffer (10 mM Tris-HCl, pH 7.5, 0.05% SDS, 25 μg/mL proteinase K (ThermoFisher Scientific)) directly into each transfected well. The mixture was incubated at 37° C. for 1 hour then heat inactivated at 80° C. for 30 minutes. Genomic DNA lysate was subsequently used immediately for high-throughput sequencing (HTS).

### Orthogonal R-Loop Assay

Orthogonal R-loop assays to measure off-target editing were performed as previously described⁵¹, with minor modifications. Under standard conditions, 200 ng of SpCas9 sgRNA plasmid, 200 ng of SaCas9 sgRNA plasmid, 300 ng of base editor plasmid, and 300 ng of dSaCas9 plasmid were co-transfected into HEK293T cells using 1.5 μL of Lipofectamine 2000. For these transfection experiments, cells were cultured for 3 days, then washed with 1× PBS (ThermoFisher Scientific), followed by genomic DNA extraction by addition of 100 μL freshly prepared lysis buffer (10 mM Tris-HCl, pH 7.5, 0.05% SDS, 25 μg/mL proteinase K (ThermoFisher Scientific)) directly into each transfected well. The mixture was incubated at 37° C. for 1 hour then heat inactivated at 80° C. for 30 minutes. Genomic DNA lysate was used immediately for high-throughput sequencing (HTS).

### Protein Purification for In Vitro Studies

SpABE7.10 and SpABE8e, which were used in in vitro deamination assays, were cloned into a pBR322 plasmid. The cloned constructs contain an N-terminal His6-tag. The proteins were expressed in E. coli strain BL21 Rosetta 2 (DE3) (EMD Biosciences) and purified as described previously⁵⁴ with a few alterations. The cells were lysed via sonication in 30 mM HEPES pH 8.0, 1 M KCl, 2 mM TCEP, 10% (v/v) glycerol, 0.5% Triton X-100, and 10 mM imidazole buffer supplemented with protease inhibitor cocktail (Roche). The proteins were purified in several steps. First, clarified lysates were loaded onto a HisTrap FF column (GE Healthcare) and proteins were eluted with a gradient of lysis buffer supplemented with up to 300 mM imidazole. Next, the eluted proteins were separated from non-specific nucleic acids using heparin HiTrap column (GE Healthcare) and eluted with a linear gradient of 100 mM to 1 M KCl. Finally, the proteins were further purified by size-exclusion chromatography using a Superdex 200 16/60 column and 20 mM HEPES pH 7.5, 400 mM KCl, 2 mM TCEP, 10% (v/v) glycerol buffer. The eluted proteins were concentrated to ˜10 mg/mL.

### Single Turnover In Vitro Deamination Assay

A single guide RNA (5′-GUUCACCUUUCUUUGUCUGUGUUUUAGAGCUAGAAAUAGCAAGUUAAAAUAAGG CUAGUCCGUUAUCAACUUGAAAAAGUG-3′ (SEQ ID NO: 312)) was transcribed in vitro using synthetic DNA oligonucleotides (IDT) containing a T7 promoter sequence. The sgRNA was purified by 12% polyacrylamide gel electrophoresis (PAGE), then extracted from the gel using the crush-and-soak method followed by ethanol precipitation and dephosphorylated using rSAP (New England Biolabs).

For in vitro deamination assays, 10 pmol of DNA oligonucleotide (non-target strand which contains a single adenine 5′-GTTCGGTGGCTCCGTCCGTGTTCACCTTTCTTTGTCTGTGGGCGTTTTGGTTGCTCTTC G-3′ (SEQ ID NO: 313)) was 5′-radiolabeled using [γ-³²P] ATP (PerkinElmer) and 5 units of T4 polynucleotide kinase (New England Biolabs) in 1′ T4 PNK buffer (New England Biolabs) at 37° C. for 30 minutes. The labeling reaction was purified using an Illustra™ MicroSpin G-25 column (GE Life Sciences) to remove free nucleotides. The dsDNA substrates were prepared by annealing the 5′-radiolabeled non-target strand and an excess of unlabeled target strand (5′-CGAAGAGCAACCAAAACGCCCACAGACAAAGAAAGGTGAACACGGACGGAGCCAC CG AAG-3′ (SEQ ID NO: 314)).

The SpABE7.10+sgRNA and SpABE8e+sgRNA RNP complexes were prepared by mixing the sgRNA and appropriate SpABE in a 1.5:1 molar ratio in RNP assembly buffer (20 mM Tris-HCl pH 7.5, 200 mM KCl, 5% (v/v) glycerol, 5 mM MgCl2, 2 mM DTT) and incubating at room temperature for 20 min.

The deamination reactions containing 1 μM RNPs in deamination buffer (20 mM Tris-HCl pH 7.5, 100 mM KCl, 5% (v/v) glycerol, 2.5 mM MgSO₄, 2 mM DTT) were initiated by adding 5′-radiolabeled dsDNA to a final concentration of 1 nM, then incubated at 37° C. 20 μL aliquots were removed at the 0, 1, 3, 8, 23, and 32 hours in case of SpABE7.10 and at the 0, 1, 2, 5, 10, 20, and 60 minutes in case of SpABE8e. The reactions were quenched by mixing them with 30 μL of hot water and incubating them at 95° C. for 2 minutes. The reactions were then purified using Monarch PCR & DNA Cleanup kit (New England Biolabs). To detect adenosine deamination, eluted DNA was incubated with 20 units of E. coli Endonuclease V (EndoV) in 1′ NEB4 buffer at 37° C. for 1 hour. EndoV specifically recognizes deaminated adenosine (inosine) in dsDNA and ssDNA and cleaves DNA one nucleotide downstream of the modification site^{43, 55, 56}. After cleavage by EndoV, the samples were mixed with an equal volume of formamide gel loading buffer (95% formamide, 100 mM EDTA, 0.025% SDS, and 0.025% (w/v) bromophenol blue), heated at 95° C. for 2 minutes, resolved on 15% denaturing polyacrylamide gel, and visualized by phosphorimaging. Assays were performed in three independent replicates, and the intensities of the uncleaved and cleaved DNA were analyzed using ImageQuant TL Software (GE Healthcare). Apparent rates were calculated by a fit to a single exponential decay (Prism7, GraphPad).

### Protein Purification for RNP Delivery to Mammalian Cells

ABE8e was codon optimized for bacterial expression and cloned into the protein expression plasmid pD881-SR (Atum, Cat. No. FPB-27E-269). The expression plasmid was transformed into BL21 Star DE3 competent cells (ThermoFisher, Cat. No. C601003). Colonies were picked for overnight growth in Terrific Broth (TB) with 25 ug/mL kanamycin and grown overnight with shaking at 37° C. The next day, 2 L of pre-warmed TB were inoculated with overnight culture at a starting OD₆₀₀ of 0.05. Cells were shaken at 37° C. for about 2.5 hours until the OD₆₀₀ was ˜1.5. Cultures were cold shocked in an ice-water slurry for 1 hour, following which D-rhamnose was added to a final concentration of 0.8% (w/v). Cultures were then incubated at 18° C. with shaking for 24 hours to induce. Following induction, cells were pelleted and flash-frozen in liquid nitrogen and stored at −80° C. The next day, cells were resuspended in 30 mL cold lysis buffer (1 M NaCl, 100 mM Tris-HCl pH 7.0, 5 mM TCEP, 20% glycerol, with 5 tablets of complete, EDTA-free protease inhibitor cocktail tablets (Millipore Sigma, Cat. No. 4693132001)). Cells were passed 3 times through a homogenizer (Avestin Emulsiflex-C3) at ˜18,000 psi for lysis. Cell debris was pelleted for 20 min using a 20,000xg centrifugation at 4° C. Supernatant was collected and spiked with 40 mM imidazole, followed by a 1 hour incubation at 4° C. with 1 mL of Ni-NTA resin slurry (G Bioscience Cat. No. 786-940, prewashed once with lysis buffer). Protein-bound resin was washed twice with 12 mL of lysis buffer in a gravity column at 4° C. Protein was eluted in 3 mL of elution buffer (300 mM imidazole, 500 mM NaCl, 100 mM Tris-HCl pH 7.0, 5 mM TCEP, 20% glycerol). Eluted protein was diluted in 40 mL of low-salt buffer (100 mM Tris-HCl, pH 7.0, 5 mM TCEP, 20% glycerol) just before loading into a 50 mL Akta Superloop for ion exchange purification on the Akta Pure25 FPLC. Ion exchange chromatography was conducted on a 5 mL GE Healthcare HiTrap SP HP pre-packed column (Cat. No. 17115201). After washing the column with low salt buffer, the diluted protein was flowed through the column to bind. The column was then washed in 15 mL of low salt buffer before being subjected to an increasing gradient to a maximum of 80% high-salt buffer (1 M NaCl, 100 mM Tris-HCl, pH 7.0, 5 mM TCEP, 20% glycerol) over the course of 50 mL, at a flow rate of 5 mL/min. 1 mL fractions were collected during this ramp to high-salt buffer. Peaks were assessed by SDS-PAGE to identify fractions containing the desired protein, which were concentrated first using an Amicon Ultra 15 mL centrifugal filter (100-kDa cutoff, Cat. No. UFC910024), followed by a 0.5 mL 100 kDa cutoff Pierce concentrator (Cat. No. 88503). Concentrated protein was quantified using a BCA assay (ThermoFisher, Cat. No. 23227); the stock concentration was 3.8 mg/mL.

### Protein Nucleofection

Nucleofection was conducted as per manufacturer's recommendations using a Lonza 4D nucleofector and the Lonza SF Cell Line 4D-Nucleofector X Kit S (Cat No. V4XC-2032). After formulating the SF nucleofection buffer, 200,000 cells were resuspended in 5 μL of buffer per nucleofection. In the remaining 15 μL of buffer per nucleofection, 20 pmol of chemically-modified sgRNA from Synthego was combined with 18 pmol of ABE8e protein and incubated 5 min at room temperature to complex. Cells were added to the 20 μL nucleofection cuvettes, followed by protein solution, pipetting up and down to mix. Cells were nucleofected with program CM-130, immediately after which 80 μL of warmed media was added to each well for recovery. After 5 min, 25 μL from each sample was added to 250 μL of fresh media in a 48-well poly-D-lysine plate (Corning). Cells were then treated the same way as lipofected cells above for genomic DNA extraction after 3 more days of culture.

### HTS of Genomic DNA Samples

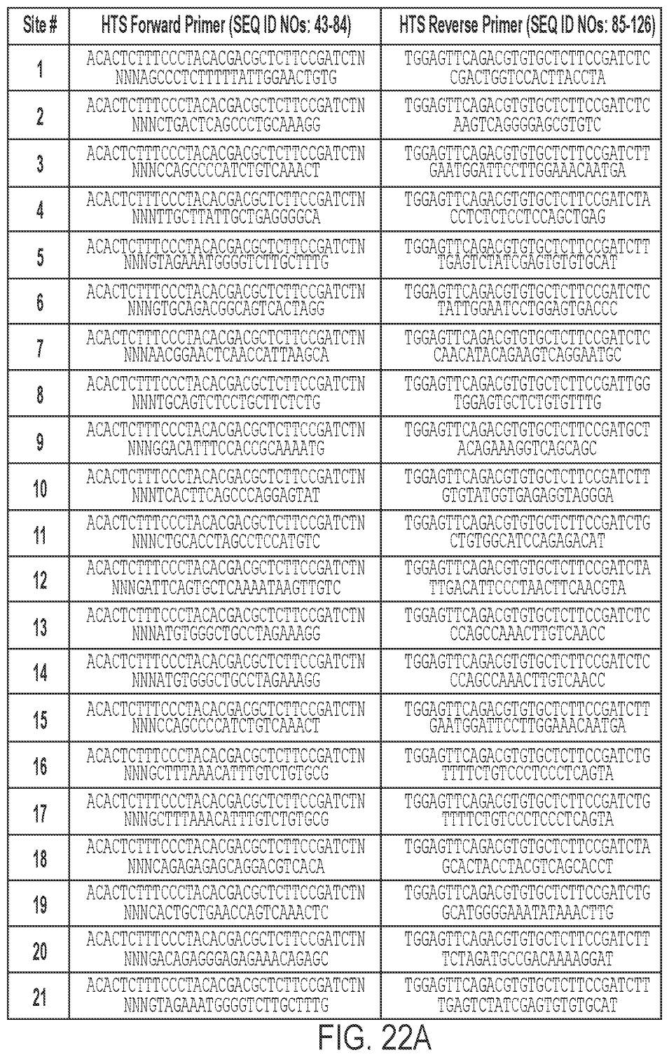

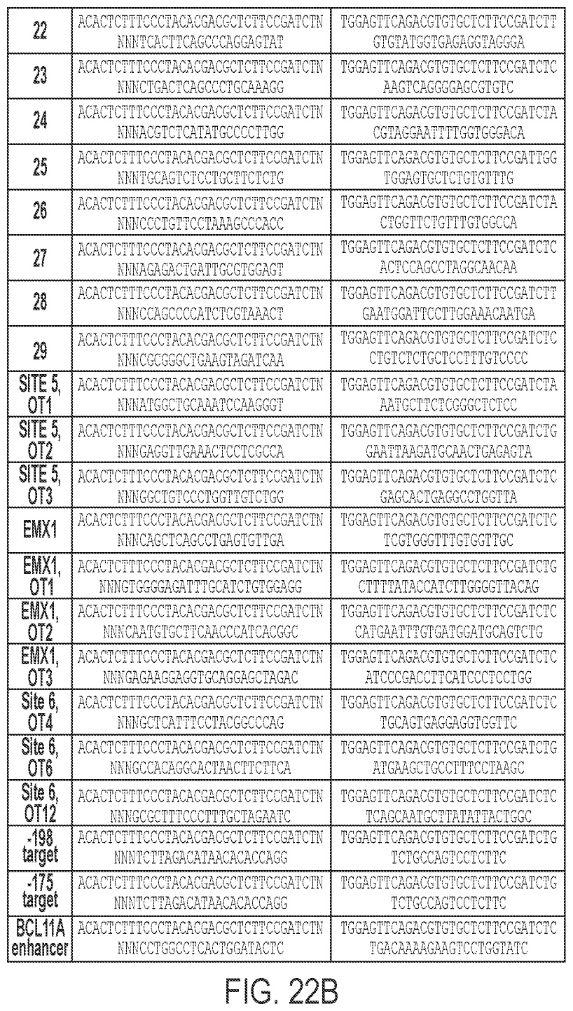

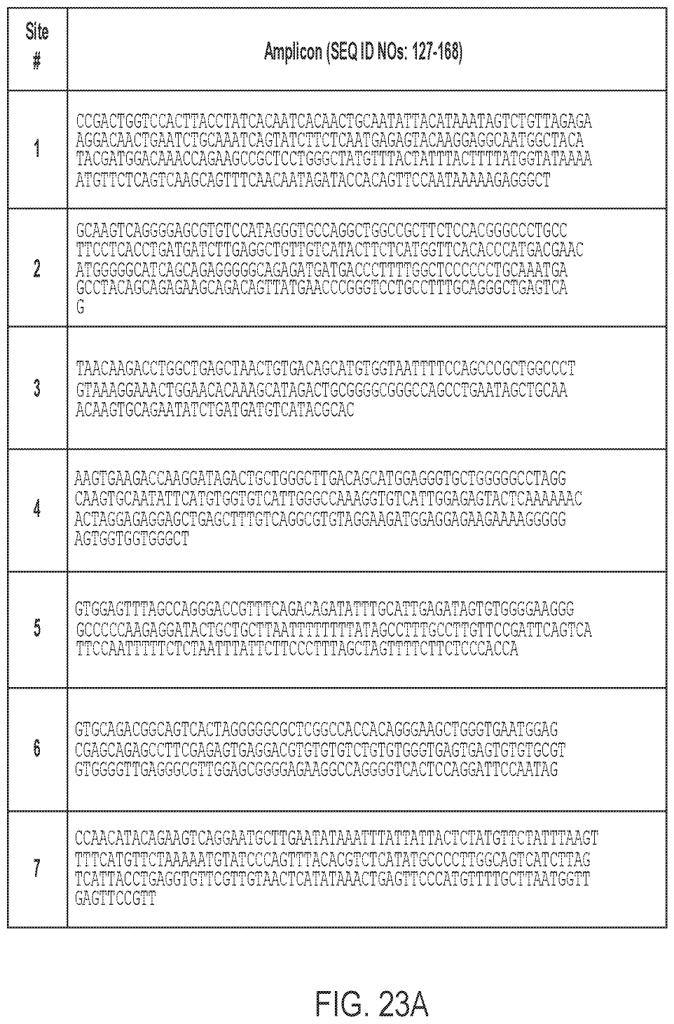

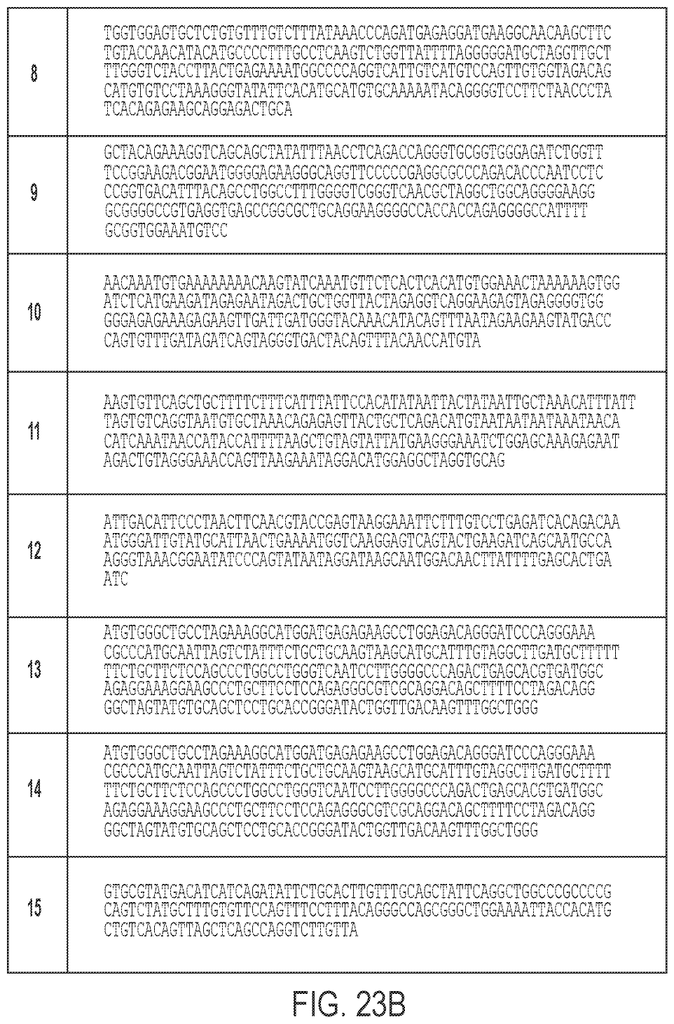

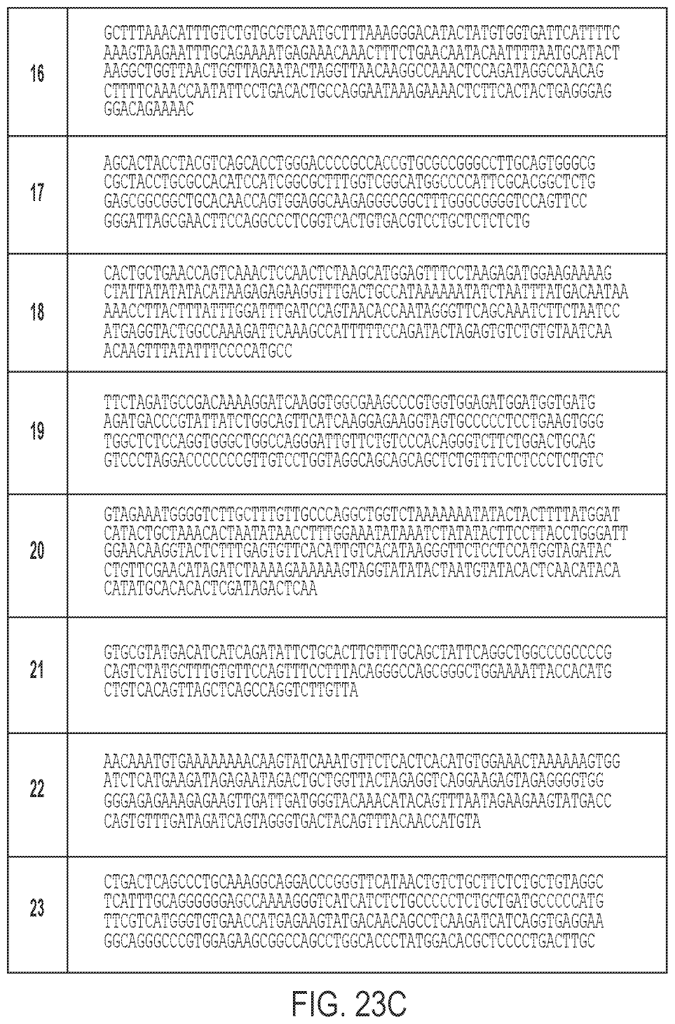

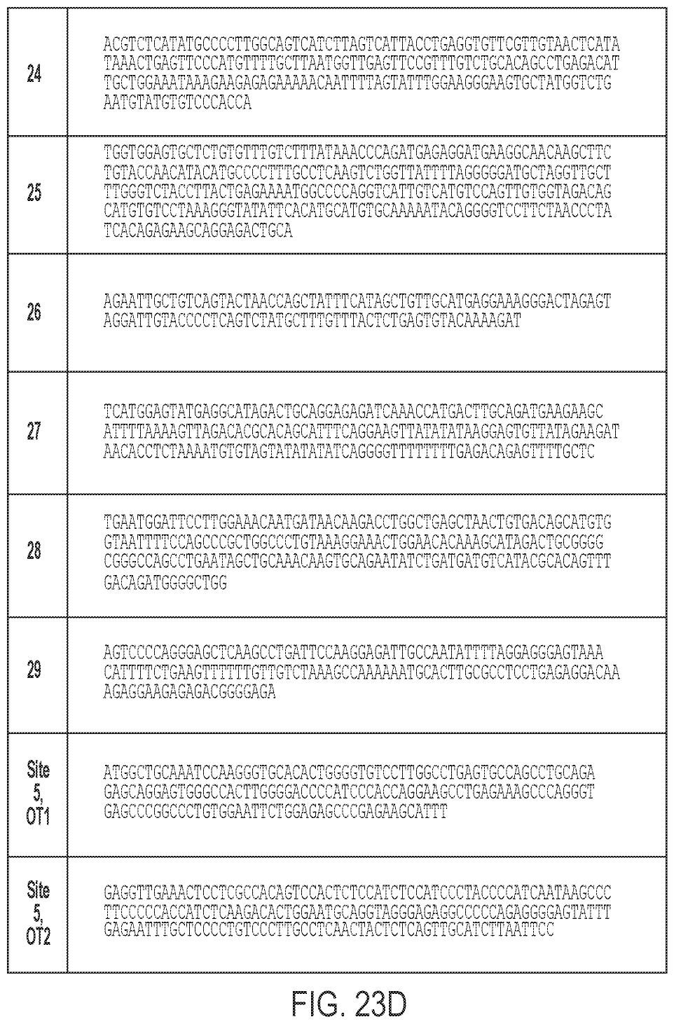

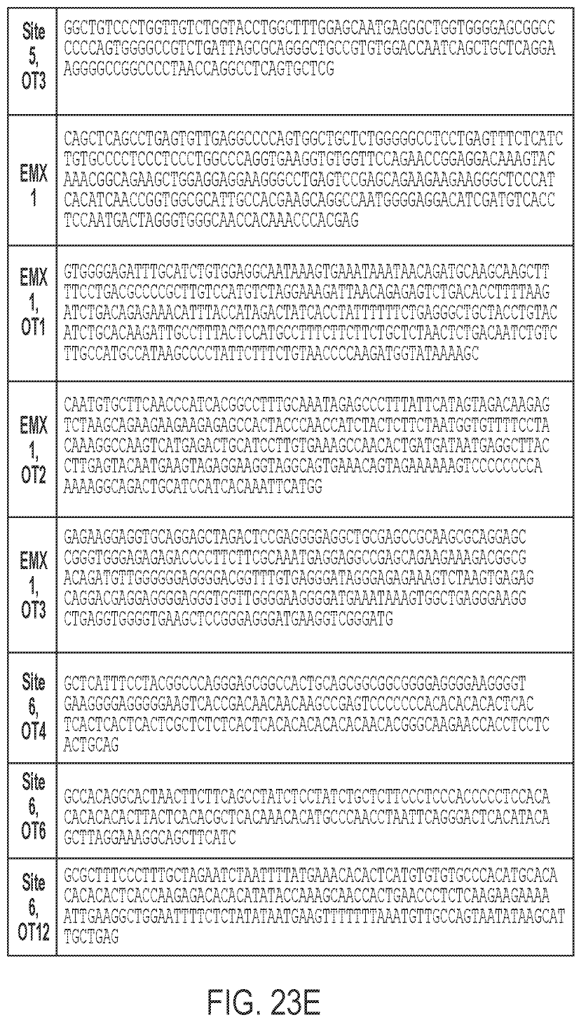

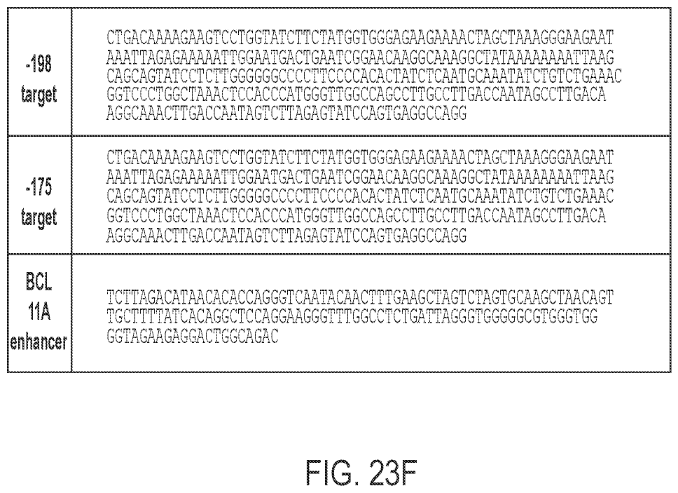

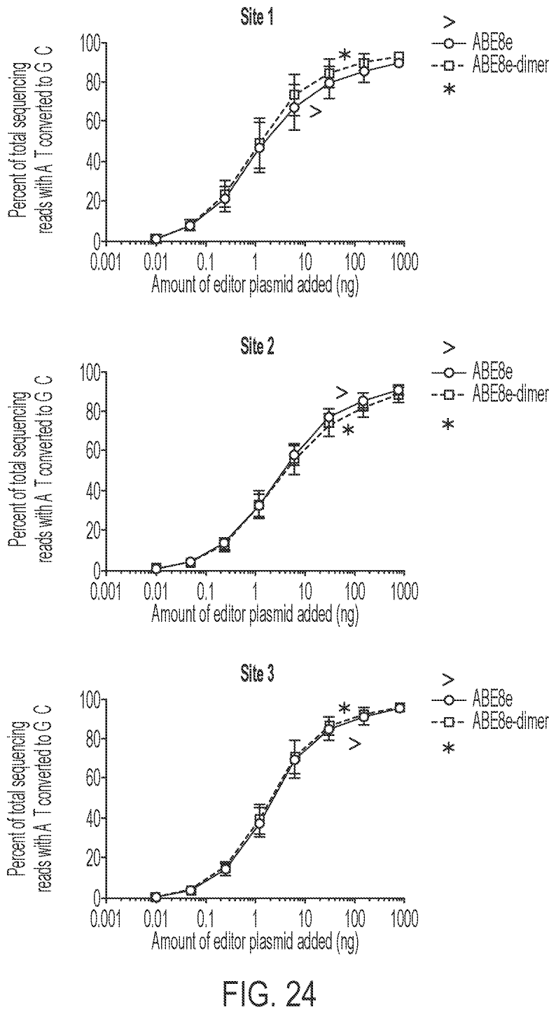

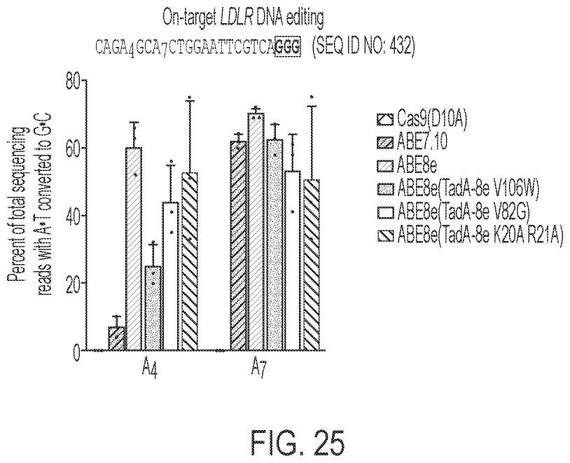

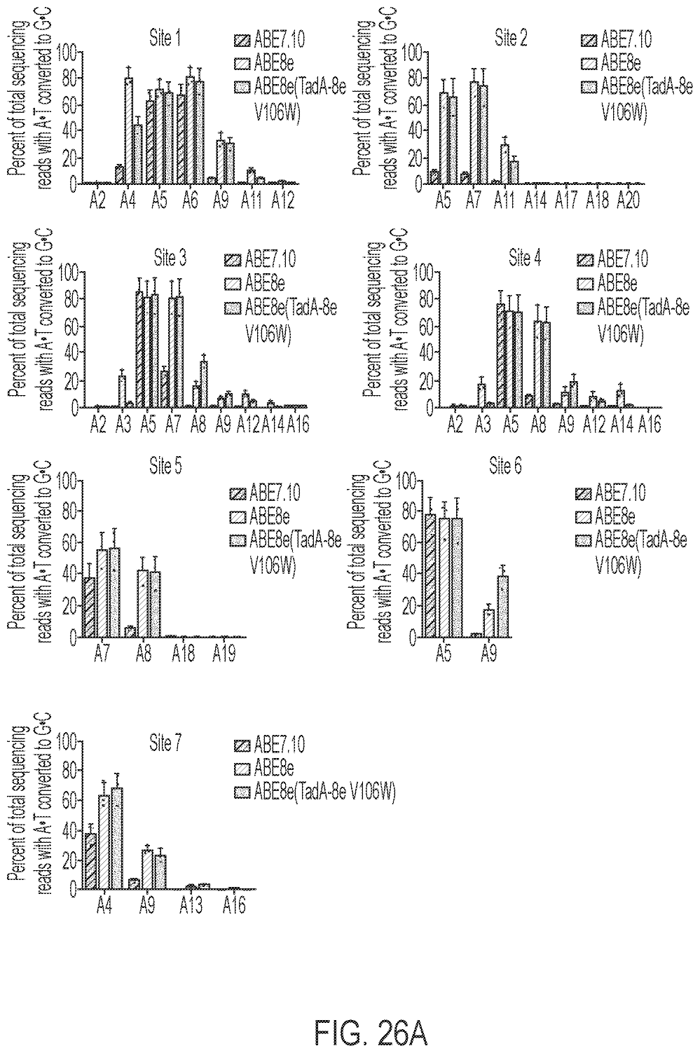

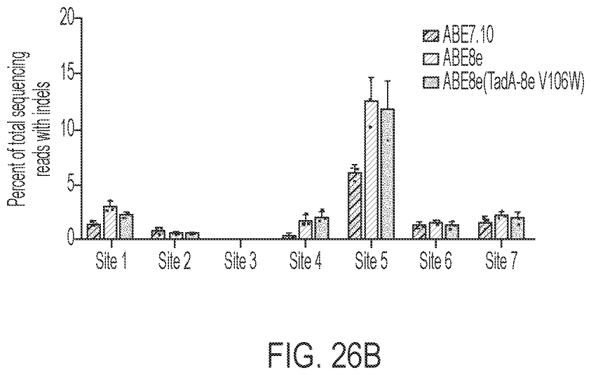

HTS of genomic DNA from HEK293T cells was performed as previously described¹. Primers for PCR 1 of target genomic site amplification are listed in FIGS. 22A-22B, and amplicons for analyses are listed in FIGS. 23A-23F. Following Illumina barcoding, PCR products were pooled and purified by electrophoresis with a 2% agarose gel using a Monarch DNA Gel Extraction Kit (New England Biolabs), eluting with 30 μl H2O. DNA concentration was quantified with Qubit dsDNA High Sensitivity Assay Kit (ThermoFisher Scientific) and sequenced on an Illumina MiSeq instrument (paired-end read, R1: 250-280 cycles, R2: 0 cycles) according to the manufacturer's protocols.

### HTS Data Analysis

Sequencing reads were demultiplexed using the MiSeq Reporter (Illumina) and FASTQ files were analyzed using CRISPResso2⁵², a publicly available algorithm that provides analyses of genome editing outcomes from deep sequencing data. See Clement et al., Nat Biotechnol. 2019 March; 37(3):224-226, herein incorporated by reference. Dual editing in individual alleles were analyzed by a Python script provided below in Supplementary Note 1. Base-editing values are representative of n=3 independent biological replicates collected by different researchers, with the mean±s.d. shown. Base-editing values are reported as a percentage of the number of reads with adenine mutagenesis over the total aligned reads. All raw FASTQ files generated are available from the NCBI SRA under BioProject PRJNA589228.

### Preparation of RNA Libraries for RNA-Seq

Polyadenylated mRNA was enriched from 1 μg of total RNA using the PolyA mRNA isolation kit (Takara) on the Apollo 324 instrument. Stranded RNA-seq libraries were generated from these samples using the PrepX mRNA 48 kit (Takara) on the Apollo 324 followed by barcoding and amplification (12 cycles). Following PCR and bead cleanup with AmpureXP beads (Beckman Coulter), libraries were visualized on a 2200 TapeStation (Agilent) and quantified using a Library Quantification Kit (KAPA Biosystems) for multiplexing. Libraries were sequenced on a NextSeq high-throughput flowcell (Illumina) as 75 bp paired-end reads. All raw FASTQ files generated are available from the NCBI SRA under BioProject PRJNA589228.

### RNA-Seq Data Analysis

Analysis of the transcriptome-wide editing RNA sequencing data was performed as follows. Prior to the analysis described below, FASTQ files were generated using Bcl2fastq2, then trimmed using Trimmomatic version 0.32 to remove adaptor sequences, unpaired sequences, and low-quality bases. Sam alignments were created using HISAT2 to align paired reads from each of three biological replicates to the hg19 human reference genome (UCSC). Precomputed HISAT2 indexes where obtained from ccb.jhu.edu/software/hisat2/index.shtml. The resulting sam files were sorted and indexed using the samtools software package.

To calculate the average % of A-to-I editing amongst adenosines sequenced in transcriptome-wide sequencing analysis, REDItools v1.3 was used to quantify the % A-to-I editing in each sample. All nucleotides were removed except adenosines from the analysis, and then removed all adenosines with a read coverage less than 10 to avoid errors due to low sampling; additionally, positions with a mapping or read quality score below 25 were removed. Next, the number of adenosines converted to an inosine in each sample was calculated and divided this by the total number of adenosines in the dataset after filtering to obtain a percentage of adenosines edited to inosine in the transcriptome. Calculation of s.e.m. was generated by comparison of 3 biological replicates.

### Reproducibility

Biologically independent experiments reported here were performed by different researchers using independent splits of the mammalian cell type used.

Supplementary Note 1—Python script to extract allele frequencies with two edits.

```
#!/usr/bin/env python
# coding: utf-8
# In[ 1]:
#import python packages import pandas as pd
import matplotlib.pyplot as plt import numpy as np
# In[2]:
#import data
#names is a tab-delimited file containing the CRISPResso2.0 allele frequency table filenames.
#here, names.txt contains 18 filenames, sorted by site, editor, and biological replicate.
names = “names.txt”
df = pd.read_table(names,delim_whitespace=True)
df.set_index(‘file’,inplace=True)
#group the files according to their corresponding genomic locus.
files_site5 = list(df.index.values)[0:6] #six files: three for three ABE7.10 replicates; three for
three ABE8e replicates.
files_site6 = list(df.index.values)[6:12]
files_site7 = list(df.index.values)[12:18]
# In[3]:
def get_data(site,D):
  “‘Function inputs: site number (here: 5,6, or 7), and allele frequencies table (D).
  Function output: the percent of total alleles in which both of the adenine (A) substrates are
  converted to the desired guanine (G) products.
Gu and Gd correspond to a G product at the upstream and downstream target positions,
respectively.
’”
if site == 5:
total_reads = D[D[“Aligned_Sequence”].str.contains(‘..............................’,
regex=True)][“#Reads”].sum( )
#Gu G at upstream position; Gd = G at downstream position
Gu_Gd = D[D[“Aligned_Sequence”].str.contains(‘...........GG.................’,
regex=True)][“#Reads”].sum( )
percent_Gu_Gd = 100*Gu_Gd/total_reads
data = {‘% of total reads with both substrates edited’: percent_Gu_Gd}
if site == 6:
total_reads = D[D[“Aligned_Sequence”].str.contains(‘..............................’,
regex=True)][“#Reads”].sum( )
Gu_Gd = D[D[“Aligned_Sequence”].str.contains(‘.........G...G................’,
regex=True)][“#Reads”].sum( )
percent_Gu_Gd = 100*Gu_Gd/total_reads
data = {‘% of total reads with both substrates edited’: percent_Gu_Gd
if site == 7:
total_reads = D[D[“Aligned_Sequence”].str.contains(‘..............................’,
regex=True)][“#Reads”].sum( )
Gu_Gd = D[D[“Aligned_Sequence”].str.contains(‘........G....G................’,
regex=True)][“#Reads”].sum( )
percent_Gu_Gd = 100*Gu_Gd/total_reads
data = {‘% of total reads with both substrates edited’: percent_Gu_Gd
return data
# In[4]:
def makedfs(site,names):
“‘Function inputs: Site number (here: 5,6, or 7) and the names.txt file.
Please note: ABE7.10 is henceforth referred to as ABE7 in the code.
Function output: Two tables, one for ABE7 and one for ABE8e, that contain the get_data
function output for
each of the three biological replicates at the designated site.
’”
#i, below, is initialized and subsequently used to track iterations of the for loop
#and to appropriately assign the allele frequency table to the corresponding editor
(ABE7.10 or ABE8e)
i = 0
global df_ABE7
global df_ABE8e
for file in names:
D = pd.read_table(file,delim_whitespace=True)
#the output after the first three iterations (0<=i<=2) is the ABE7.10 dataframe
if i == 0:
    df_ABE7 = pd.DataFrame(get_data(site,D),index=[str(file)])
if 1 <= i <= 2:
    D_output_new = pd.DataFrame(get_data(site,D),index=[str(file)]) df_ABE7 =
pd.concat([df_ABE7, D_output_new], axis =0)
#the output after the final three iterations (3<=i<=5) is the ABE8e dataframe
if i == 3:
    df_ABE8e=pd.DataFrame(get_data(site,D),index=[str(file)]) if 4<=i<=5:
D_output_new = pd.DataFrame(get_data(site,D),index=[str(file)]) df_ABE8e=
pd.concat([df_ABE8e, D_output_new], axis =0)
i+=1 #tracking iteration
    return df_ABE7,df_ABE8e
# In[5]:
#create dataframes
ABE7Site5,ABE8eSite5 = makedfs(5,files_site5)
ABE7Site6,ABE8eSite6 = makedfs(6,files_site6)
ABE7Site7,ABE8eSite7 = makedfs(7,files_site7)
# In[6]:
#for the given editor and site, obtain the average, across three biologial replicates,
#percent of total alleles in which both substrate bases are converted.
ABE7_Site5_GuGd_average = float(ABE7Site5.mean( ))
ABE8e_Site5_GuGd_average = float(ABE8eSite5.mean( ))
ABE7_Site6_GuGd_average = float(ABE7Site6.mean( ))
ABE8e_Site6_GuGd_average = float(ABE8eSite6.mean( ))
ABE7_Site7_GuGd_average = float(ABE7Site7.mean( ))
ABE8e_Site7_GuGd_average = float(ABE8eSite7.mean( ))
#store averages by editor for subsequent plotting
means_ABE7 =
(ABE7_Site5_GuGd_average,ABE7_Site6_GuGd_average,ABE7_Site7_GuGd_average)
means_ABE8e =
(ABE8e_Site5_GuGd_average,ABE8e_Site6_GuGd_average,ABE8e_Site7_GuGd_average)
#for the given editor and site, obtain the standard deviation, across three
#biological replicates, in the percent of total alleles in which both substrate bases are converted.
ABE7_Site5_GuGd_std = float(ABE7Site5.std( ))
ABE8e_Site5_GuGd_std = float(ABE8eSite5.std( ))
ABE7_Site6_GuGd_std = float(ABE7Site6.std( ))
ABE8e_Site6_GuGd_std = float(ABE8eSite6.std( ))
ABE7_Site7_GuGd_std = float(ABE7Site7.std( ))
ABE8e_Site7_GuGd_std = float(ABE8eSite7.std( ))
#store standard deviations by editor for subsequent plotting
std_ABE7 = (ABE7_Site5_GuGd_std,ABE7_Site6_GuGd_std,ABE7_Site7_GuGd_std)
std_ABE8e = (ABE8e_Site5_GuGd_std,ABE8e_Site6_GuGd_std,ABE8e_Site7_GuGd_std)
# In[7]:
#number of sites n_sites = 3
#create plot
fig, ax = plt.subplots( )
index = np.arange(n_sites)
bar_width = 0.35
opacity = 1
#plot data
ABE7bars = plt.bar(index, means_ABE7,
bar_width,alpha=opacity,color=‘#A0A0A3’,label=‘ABE7.10’,yerr=std_ABE7,capsize=5)
ABE8ebars = plt.bar(index + bar_width, means_ABE8e, bar_width,
alpha=opacity,color=‘#F94040’,label=‘ABE8e’,yerr=std_ABE8e,capsize=5)
#label plot
plt.ylabel(‘Percent of total alleles in which both ¥n substrate bases are converted’)
plt.xticks(index + bar_width/2, (‘Site 5’, ‘Site 6’, ‘Site 7’))
#display plot with legend
plt.legend( )
plt.tight_layout( )
plt.show( )
# In[ ]:
```

### REFERENCES FOR EXAMPLES 1-4

1. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
2. Gaudelli, N. M. et al. Programmable base editing of A*T to G*C in genomic DNA without DNA cleavage. Nature 551, 464-471 (2017).
3. Rees, H. A. & Liu, D. R. Base editing: precision chemistry on the genome and transcriptome of living cells. Nat. Rev. Genet. 19, 770-788 (2018).
4. Landrum, M. J. et al. ClinVar: public archive of interpretations of clinically relevant variants. Nucleic Acids Res. 44, D862-868 (2016).
5. Landrum, M. J. et al. ClinVar: public archive of relationships among sequence variation and human phenotype. Nucleic Acids Res. 42, D980-985 (2014).
6. Hu, J. H. et al. Evolved Cas9 variants with broad PAM compatibility and high DNA specificity. Nature 556, 57-63 (2018).
7. Huang, T. P. et al. Circularly permuted and PAM-modified Cas9 variants broaden the targeting scope of base editors. Nat. Biotechnol. 37, 626-631 (2019).
8. Kim, Y. B. et al. Increasing the genome-targeting scope and precision of base editing with engineered Cas9-cytidine deaminase fusions. Nat. Biotechnol. 35, 371-376 (2017).
9. Kleinstiver, B. P. et al. Engineered CRISPR-Cas12a variants with increased activities and improved targeting ranges for gene, epigenetic and base editing. Nat. Biotechnol. 37, 276-282 (2019).
10. Li, X. et al. Base editing with a Cpf1-cytidine deaminase fusion. Nat. Biotechnol. 36, 324-327 (2018).
11. Nishimasu, H. et al. Engineered CRISPR-Cas9 nuclease with expanded targeting space. Science 361, 1259-1262 (2018).
12. Esvelt, K. M., Carlson, J. C. & Liu, D. R. A system for the continuous directed evolution of biomolecules. Nature 472, 499-503 (2011).
13. Badran, A. H. & Liu, D. R. In vivo continuous directed evolution. Curr. Opin. Chem. Biol. 24, 1-10 (2015).
14. Nishida, K. et al. Targeted nucleotide editing using hybrid prokaryotic and vertebrate adaptive immune systems. Science 353 (2016).
15. Komor, A. C. et al. Improved base excision repair inhibition and bacteriophage Mu Gam protein yields C:G-to-T:A base editors with higher efficiency and product purity. Sci. Adv. 3, eaao4774 (2017).
16. Koblan, L. W. et al. Improving cytidine and adenine base editors by expression optimization and ancestral reconstruction. Nat. Biotechnol. 36, 843-846 (2018).
17. Komor, A. C., Badran, A. H. & Liu, D. R. Editing the genome without double-stranded DNA breaks. ACS Chem. Biol. 13, 383-388 (2018).
18. Villiger, L. et al. Treatment of a metabolic liver disease by in vivo genome base editing in adult mice. Nat. Med. 24, 1519-1525 (2018).
19. Ryu, S. M. et al. Adenine base editing in mouse embryos and an adult mouse model of Duchenne muscular dystrophy. Nat. Biotechnol. 36, 536-539 (2018).
20. Yeh, W. H., Chiang, H., Rees, H. A., Edge, A. S. B. & Liu, D. R. In vivo base editing of post-mitotic sensory cells. Nat. Commun. 9, 2184 (2018).
21. Tanaka, S. et al. In vivo targeted single-nucleotide editing in zebrafish. Sci. Rep. 8, 11423 (2018).
22. Zhang, Y. et al. Programmable base editing of zebrafish genome using a modified CRISPR-Cas9 system. Nat. Commun. 8, 118 (2017).
23. Ma, Y. et al. Highly efficient and precise base editing by engineered dCas9-guide tRNA adenosine deaminase in rats. Cell Discov. 4, 39 (2018).
24. Zafra, M. P. et al. Optimized base editors enable efficient editing in cells, organoids and mice. Nat. Biotechnol. 36, 888-893 (2018).
25. Liu, Z. et al. Efficient generation of mouse models of human diseases via ABE- and BE-mediated base editing. Nat. Commun. 9, 2338 (2018).
26. Liu, Z. et al. Highly efficient RNA-guided base editing in rabbit. Nat. Commun. 9, 2717 (2018).
27. Song, C.-Q. et al. Adenine base editing in an adult mouse model of tyrosinaemia. Nat. Biomed. Eng. (2019).
28. Yourik, P., Fuchs, R. T., Mabuchi, M., Curcuru, J. L. & Robb, G. B. Staphylococcus aureus Cas9 is a multiple-turnover enzyme. RNA 25, 35-44 (2019).
29. Bryson, D. I. et al. Continuous directed evolution of aminoacyl-tRNA synthetases. Nat Chem Biol. 13, 1253-1260 (2017).
30. Carlson, J. C., Badran, A. H., Guggiana-Nilo, D. A. & Liu, D. R. Negative selection and stringency modulation in phage-assisted continuous evolution. Nat. Chem. Biol. 10, 216-222 (2014).
31. Dickinson, B. C., Leconte, A. M., Allen, B., Esvelt, K. M. & Liu, D. R. Experimental interrogation of the path dependence and stochasticity of protein evolution using phage-assisted continuous evolution. Proc. Natl. Acad. Sci. U.S.A. 110, 9007-9012 (2013).
32. Dickinson, B. C., Packer, M. S., Badran, A. H. & Liu, D. R. A system for the continuous directed evolution of proteases rapidly reveals drug-resistance mutations. Nat. Commun. 5, 5352 (2014).
33. Hubbard, B. P. et al. Continuous directed evolution of DNA-binding proteins to improve TALEN specificity. Nat. Methods 12, 939-942 (2015).
34. Leconte, A. M. et al. A population-based experimental model for protein evolution: effects of mutation rate and selection stringency on evolutionary outcomes. Biochemistry 52, 1490-1499 (2013).
35. Packer, M. S., Rees, H. A. & Liu, D. R. Phage-assisted continuous evolution of proteases with altered substrate specificity. Nat. Commun. 8, 956 (2017).
36. Wang, T., Badran, A. H., Huang, T. P. & Liu, D. R. Continuous directed evolution of proteins with improved soluble expression. Nat. Chem. Biol. 14, 972-980 (2018).
37. Roth, T. B., Woolston, B. M., Stephanopoulos, G. & Liu, D. R. Phage-Assisted Evolution of Bacillus methanolicus Methanol Dehydrogenase 2. ACS Synth. Biol. 8, 796-806 (2019).
38. Thuronyi, B. W. et al. Continuous evolution of base editors with expanded target compatibility and improved activity. Nat. Biotechnol., 1070-1079 (2019).
39. Badran, A. H. et al. Continuous evolution of Bacillus thuringiensis toxins overcomes insect resistance. Nature 533, 58-63 (2016).
40. Badran, A. H. & Liu, D. R. Development of potent in vivo mutagenesis plasmids with broad mutational spectra. Nat. Commun. 6, 8425 (2015).
41. Swarts, D. C., van der Oost, J. & Jinek, M. Structural Basis for Guide RNA Processing and Seed-Dependent DNA Targeting by CRISPR-Cas12a. Mol. Cell 66, 221-233 (2017).
42. Grunewald, J. et al. CRISPR DNA base editors with reduced RNA off-target and self-editing activities. Nat. Biotechnol. 37, 1041-1048 (2019).
43. Liang, P. et al. Genome-wide profiling of adenine base editor specificity by EndoV-seq. Nat. Commun. 10, 67 (2019).
44. Tsai, S. Q. et al. GUIDE-seq enables genome-wide profiling of off-target cleavage by CRISPR-Cas nucleases. Nat. Biotechnol. 33, 187-197 (2015).
45. Rees, H. A. et al. Improving the DNA specificity and applicability of base editing through protein engineering and protein delivery. Nat. Commun. 8, 15790 (2017).
46. Grunewald, J. et al. Transcriptome-wide off-target RNA editing induced by CRISPR-guided DNA base editors. Nature 569, 433-437 (2019).
47. Rees, H. A., Wilson, C., Doman, J. L. & Liu, D. R. Analysis and minimization of cellular RNA editing by DNA adenine base editors. Sci. Adv. 5, eaax5717 (2019).
48. Zhou, C. et al. Off-target RNA mutation induced by DNA base editing and its elimination by mutagenesis. Nature 571, 275-278 (2019).
49. Amato, A. et al. Interpreting elevated fetal hemoglobin in pathology and health at the basic laboratory level: new and known gamma-gene mutations associated with hereditary persistence of fetal hemoglobin. Int. J. Lab. Hematol. 36, 13-19 (2014).
50. Canver, M. C. et al. BCL11A enhancer dissection by Cas9-mediated in situ saturating mutagenesis. Nature 527, 192-197 (2015).
51. Doman, J. L. R., A.; Newby, G. A.; Liu, D. R. Evaluation and minimization of Cas9-Independent off-target DNA editing by cytosine base editors. Nat. Biotechnol. in press (2019).
52. Clement, K. et al. CRISPResso2 provides accurate and rapid genome editing sequence analysis. Nat. Biotechnol. 37, 224-226 (2019).

### Example 5: Application of ABE8e Base Editors to Rescue of Sickle Cell Disease in Human Hematopoietic Stem Cells and in Mice

Sickle cell disease (SCD) is a common, catastrophic disorder caused by a missense mutation in the HBB gene encoding the β-globin subunit of adult hemoglobin1. An adenine base editor variant (ABE8e-NRCH) with laboratory-evolved deaminase and laboratory-evolved Cas9 domains^{2, 3} was used to convert the SCD allele (HBB^{S}) to hemoglobin Makassar (HBB^{G}), a naturally occurring non-pathogenic allele^{4, 5}. Delivery of mRNA encoding ABE8e-NRCH and an HBB^{S}-targeting single guide RNA (sgRNA) into CD34+ hematopoietic stem and progenitor cells (HSPCs) from three patients with SCD using a clinically validated electroporation method resulted in 80% average conversion of HBB^{S} to HBB^{G} with no detectable perturbation of erythropoiesis. Reticulocytes differentiated from edited CD34+ cells showed an 80% shift of total β-like globin protein from sickle ((3S) to Makassar ((3G), and a 3-fold decrease in hypoxia-induced sickling. Sixteen weeks after transfer of edited human CD34+ cells into adult immunodeficient mice, the HBB^{S}-to-HBB^{S} editing frequency in engrafted cells was 68%, indicating durable modification of HSCs. Donor-derived reticulocytes isolated from the bone marrow of engrafted mice showed a shift of 63% of total β-like protein from PS to PG, and a 5-fold decrease in hypoxia-induced sickling. Since human HSPCs do not develop into durable circulating red blood cells in mice, ABE8e-NRCH and sgRNA ribonucleoprotein were also electroporated into HSPCs from a mouse harboring human SCD alleles, resulting in 56% conversion of HBB^{S} to HBB^{S}. Sixteen weeks after transplantation of these edited mouse HSPCs into irradiated adult congenic mice, circulating red blood cells (RBCs) showed a shift of 84% of total β-like protein from PS to PG, and a 3-fold decrease in hypoxia-induced sickling. Mice that received base-edited SCD HSPCs showed rescue of hematologic parameters to near-normal levels and substantially reduced splenic pathology compared to mice transplanted with unmodified HSPCs. These findings suggest a potential one-time autologous treatment for individuals with SCD that both eliminates pathogenic HBB^{S} and generates benign HBB^{S}.

Sickle cell disease (SCD) is an autosomal recessive disorder caused by an A-to-T substitution in the HBB gene, which normally encodes the adult β-globin subunit (β^{A}) of the hemoglobin tetramer, HbA (α₂β₂). At low oxygen concentrations, the mutant β-globin subunit (β^{S}) causes the sickle hemoglobin tetramer (HbS) to form stiff polymers within red blood cells (RBCs) resulting in the characteristic sickle shape and triggering a pathophysiologic cascade of hemolysis, inflammation, dysregulated nitric oxide homeostasis, and microvascular occlusion. Clinical manifestations include anemia, severe acute and chronic pain, immunodeficiency with susceptibility to sepsis, multi-organ failure and early death¹. Allogeneic hematopoietic stem cell (HSC) transplantation can cure SCD, although most patients do not have suitable HLA-matched donors and the procedure can lead to immune complications including graft rejection and graft-versus-host disease⁶.

Ex vivo modification of autologous HSCs to correct the SCD mutation or circumvent its deleterious effects forms the basis for numerous experimental therapies⁷⁻¹¹. Approaches showing early promise in clinical trials include the ectopic expression of an anti-sickling β-like globin gene by lentiviral vectors¹² or suppression of the BCL11A gene to reactivate fetal hemoglobin (HbF, α₂β₂) production, either by a lentiviral vector-expressed short hairpin RNA against BCL11A13 or by Cas9-mediated disruption of the BCL11A erythroid-specific enhancer¹⁴. These approaches, while promising, have limitations. Lentiviral vectors carry risks of insertional mutagenesis and may not effectively suppress the expression of pathological HbS. Induction of HbF does not eliminate the production of HbS, and the level of HbF required to rescue SCD is not established and is difficult to address in mouse models, where hemoglobin regulatory elements differ from those in human patients. Transplantation of CD34+ cells into immunocompromised mice can be used to examine HbF induction in erythroid precursors but cannot be used to assess hematological correction since circulating human RBCs are very short-lived in mice¹⁵. Eliminating the root cause of SCD by converting the HBB^{S} allele to a benign variant without perturbing native regulation of globin genes could address these limitations. Cas9-mediated HDR can correct HBB^{S}, but is difficult to achieve efficiently in repopulating HSCs^{16, 17} and carries the risk of uncontrolled editing byproducts mediated by double-stranded DNA breaks (DSBs) including indels, translocations, and loss of large chromosomal segments¹⁸⁻²³, as well as other potentially deleterious cell-state changes such as p53 activation²²⁻²⁵.

Adenine base editors (ABEs) convert A•T base pairs to G•C at targeted locations in the genome of living cells without requiring DSBs or donor DNA templates and with minimal indel formation^{2, 26-29}. In SCD, the normal GAG (Glu) codon encoding amino acid 6 of β-globin is mutated to GTG (Val). While adenine base editing cannot reverse the SCD mutation to the wild-type allele, it can in principle convert the pathogenic codon to GCG (Ala). The resulting HBB allele is a non-pathogenic variant termed Hb-Makassar (HBB⁶)³⁻⁵ (FIG. 30A), one of hundreds of documented naturally occurring HBB alleles^{3, 31}.

The ABE8e-NRCH monomer base editor was used to convert the SCD allele to the non-pathogenic HBB^{G} Makassar allele in mammalian cells with minimal non-synonymous editing outcomes. After introducing ABE8e-NRCH mRNA or RNP into CD34+ hematopoietic stem and progenitor cells (HSPCs) from individuals with SCD, up to 80% allelic conversion of HBB^{S} to HBB^{G} was achieved. In vitro differentiation of edited HSPCs generated erythroid cells with 81% reduced βS protein that was replaced by Makassar β-globin ((3G). The edited HSPCs engrafted into adult mice with equal efficiency as unedited control HSPCs, and with no apparent change in differentiation potential. The edited HBB^{G} allele was durable in mice (68% HBB^{G} 16 weeks after transplantation), and erythroid cells from transplanted animals showed markedly reduced hypoxia-induced sickling compared to cells derived from unedited HSCs. To assess correction of blood phenotypes and SCD rescue in an animal, ex vivo HSPCs were edited from a mouse SCD mode132 in which endogenous globin genes were replaced by human HBB^{S} and an autologous bone marrow transplant of these edited cells was simulated into irradiated recipient adult mice. Sixteen weeks after transplantation of edited HBB^{S/S} mouse HSPCs, >95% of circulating mouse blood was donor-derived cells, with an average of 46% HBB^{S}-to-HBB^{G} conversion. Total β-like globin in these animals averaged 84% PG. Animals receiving edited cells showed substantial rescue of spleen pathology, RBC sickling, and reticulocyte count, as well as restoration of total hemoglobin, RBC count, and hematocrit to near-normal levels. These findings suggest autologous ex vivo base editing and transplantation of HSCs as a potential one-time treatment for SCD.

### Results

### Evaluation of Editing Strategies to Modify the Sickle Cell Mutation

Placing the pathogenic HBB^{S} mutation within the activity window of an adenine base editor requires engaging a non-canonical CACC protospacer-adjacent motif (PAM) (FIG. 30A). To address this limitation, an evolved a Cas9 domain was used that was previously developed to recognize NRCH PAM sites (N=A, C, G, or T; R=A or G; and H=A, C, or T)3. Separately, phage-assisted continuous evolution (PACE) was used to evolve deoxyadenosine deaminases such as TadA-8e that support much higher base editing efficiencies than ABE7.10max2. To achieve highly efficient editing of HBB^{S}, the TadA-8e deaminase was combined with the NRCH PAM-targeting Cas9 nickase domain to generate ABE8e-NRCH. ABE8e-NRCH plasmid lipofection converted the HBB^{S} allele to HBB^{G} in HEK293T cells with an efficiency of 58±6.9% as measured by high-throughput DNA sequencing (HTS), 1.7-fold higher than that of ABE7.10max-NRCH (FIG. 34). Beyond conversion of the pathogenic A•T (Val) at protospacer position 7 to G•C (Ala), all other observed bystander edits in the target protospacer following ABE8e-NRCH treatment were silent (A•T-to-G•C conversions at positions 9 and 12, where the CACC PAM begins at protospacer position 21) (FIG. 30A). No bystander editing was observed at the nearest A•T base pair that would result in a non-silent edit (position 16). These results establish ABE8e-NRCH as a base editor that uses a recently evolved Cas9 variant to optimally position a high-activity laboratory-evolved deoxyadenosine deaminase to initiate conversion of the pathogenic A•T base pair.

Next, ABE8e-NRCH was used to edit human HSPCs via electroporation either of ABE8e-NRCH protein complexed with the HBB^{S}-targeting sgRNA as a ribonucleoprotein (RNP), or of ABE8e-NRCH mRNA and the sgRNA28. ABE8e-NRCH protein was purified and complexed with the single guide RNA (sgRNA) that targets the HBB^{S} allele to generate ABE8e-NRCH RNP. Poly-A tailed ABE8e-NRCH mRNA was produced from in vitro transcription and co-transcriptional capping. The synthetic sgRNA used in both RNP and mRNA formulations contained 2′-O-methyl modifications in the first three and last three nucleosides, and phosphorothioate linkages following each of the first three and preceding each of the last three nucleosides in order to enhance stability and reduce toxicity³³. MaxCyte electroporation was chosen to deliver ABE8e-NRCH RNP or mRNA because of the established clinical pathway for this procedure in ex vivo blood cell modification.

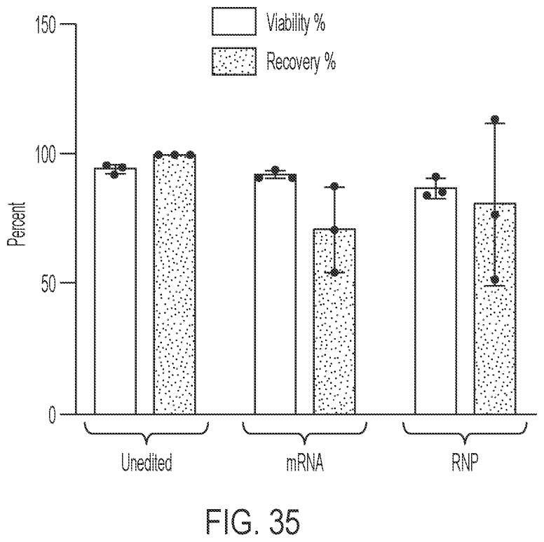

Electroporating ABE8e-NRCH RNP into plerixafor-mobilized peripheral blood CD34+ HSPCs cells from three separate SCD patient donors resulted in 44±5.9% editing of HBB^{S} to HBB^{G} with no other missense mutations detected and low indels (1.2±0.33%) 6 days after electroporation (FIG. 30B). Electroporating ABE8e-NRCH mRNA and sgRNA into the same cells resulted in 80±2.1% editing of HBB^{S} to HBB^{G} with no detectable missense mutations and 2.8±0.50% indels (FIGS. 30B and 35). Thus, introduction of ABE8e-NRCH RNP or mRNA using a clinically relevant delivery protocol can efficiently convert HBB^{S} to HBB^{G} in HSPCs from individuals with SCD.

Erythrocyte Properties after In Vitro Differentiation of Edited HSPCs

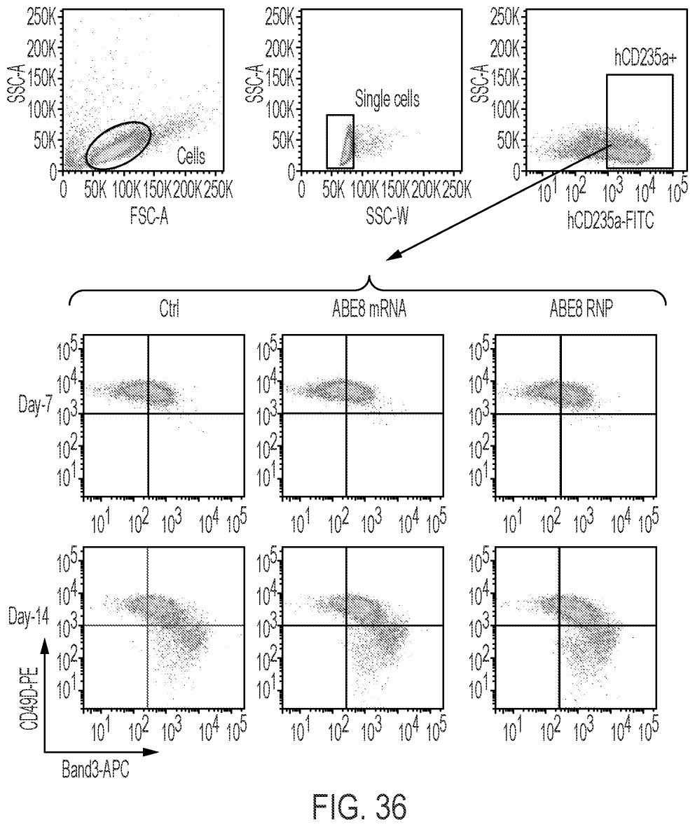

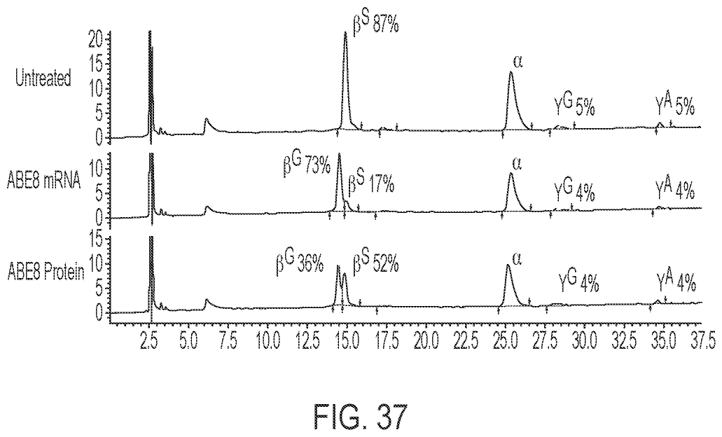

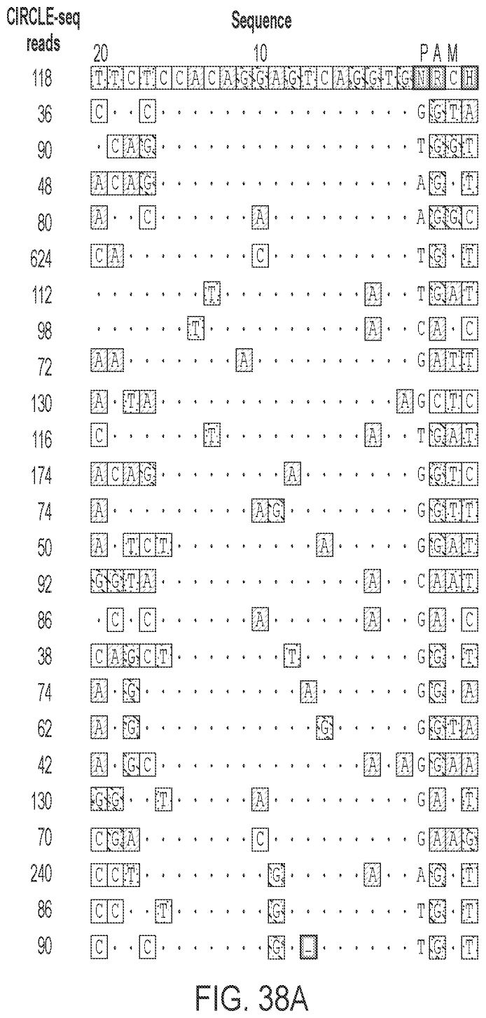

To determine whether editing led to the desired shift of erythrocyte β-globin protein from βS to βG, SCD patient CD34+ HSPCs edited with ABE8e-NRCH mRNA or RNP were differentiated in vitro over 18 days into late-stage erythroid cells (FIG. 36, Table 5)^{34, 35}. These cells were lysed and reverse-phase high performance liquid chromatography (HPLC) was performed to quantify cytoplasmic β-like globin proteins (FIG. 37). Untreated SCD patient CD34+ cells generated erythroid cells containing 87±1.3% PS and no detectable PG. In contrast, SCD patient CD34+ cells treated with ABE8e-NRCH mRNA generated reticulocytes with 72±3.0% PG and only 17±3.0% PS, representing a 5.1-fold (81%) decrease of the pathogenic (3-globin protein (FIG. 30C). The remaining β-like globins were composed of γ and δ globin, which are generally present at low basal levels. These findings show that ABE8e-NRCH-mediated editing of the HBB^{S} allele results in a substantial loss of PS protein, with concomitant production of PG to represent the majority of total cellular β-like globin protein.

| | | | | Reference |
|---|---|---|---|---|
| | | | | (PMID |
| Antibody and | | | | using this |
| fluorophore | Clone | Vendor | Catalog # | antibody) |
| Erythropoeisis | | | | |
| Anti-Human CD235a | GA-R2 (HIR2) | BD | 559943 | 31698466 |
| FITC | | Pharmingen ™ | | |
| Hoechst 33342 | Stock 10 mM = | Millipore Sigma | B2261 | 31698466 |
| | 2000× | | | |
| Anti-Human CD49d | 9F10 | BioLegend | 304304 | 31698466 |
| PE | | | | |
| Anti-Human Band3 | | New York Blood | Gift from | 31698466 |
| APC | | Center | X. An | |
| Chimerism after | | | | |
| human/NBSGW | | | | |
| xenotransplantation | | | | |
| Anti-Mouse CD45 | 30-F11 (RUO)/ | BD | 561088/ | 11062533/ |
| FITC/BV786 | 30-F11 (RUO) | Pharmingen ™/ | 564225 | 31698466 |
| | | BD Horizon ™ | | |
| Anti-Mouse TER- | TER-119 | BD | 560512 | 31698466 |
| 119/Erythroid Cells | | Pharmingen ™ | | |
| Per CP-Cy ™ 5.5 | | | | |
| Anti-Human CD45 | HI30 | BD Horizon ™ | 564047 | 31698466 |
| BV605 | | | | |
| Anti-Human CD33 | P 67.6 | BD Biosciences | 333946 | 31698466 |
| PE-Cy ™ 7 | | | | |
| Anti-Human CD3 | SK7 (Leu-4) | BD | 557832 | 31698466 |
| APC-Cy ™ 7 | (RUO) | Pharmingen ™ | | |
| Anti-Human CD19 | 4G7 (IVD)/ | BD | 349209/ | 31698466/ |
| (Leu ™-12) PE/FITC | HIB19(RUO) | Biosciences/ | 555412 | 7690791 |
| | | BD Pharmingen ™ | | |
| Anti-Human CD34 | 581 (RUO)/ | BD | 561440/ | 7680152 |
| Alexa Flour 700/PE | 581 (RUO) | Pharmingen ™/ | 555822 | |
| | | BD Pharmingen ™ | | |
| Anti-Human CD235a | GA-R2 (HIR2) | BD | 551336 | 31698466 |
| APC | (RUO) | Pharmingen ™ | | |
| Chimerism after | | | | |
| Townes SCD mouse | | | | |
| transplantation | | | | |
| Anti-mouse CD45.1 | A20 | BD Biosciences | 553776 | 28615666 |
| PE | | | | |
| Anti-mouse CD45.2 | 104 | BD Biosciences | 561874 | 28615666 |
| FITC | | | | |

Table 5. Antibodies Used in this Study.

Next, it was assessed whether the replacement of βS with βG reduced erythroid cell sickling caused by polymerization of deoxygenated hemoglobin. Purified reticulocytes generated from in vitro differentiation of unedited or ABE8e-NRCH edited SCD patient CD34+ cells were incubated in 2% oxygen for 8 hours. While reticulocytes from unedited cells showed sickled morphology with 47.7% frequency, reticulocytes from edited CD34+ HSPCs exhibited a 16.3% sickled frequency, a 2.9-fold reduction (FIGS. 30D-30E). These data confirm that conversion of HBB^{S} to HBB^{G} reduces sickling. Erythroid cells differentiated from CD34+ cells treated with ABE8e-NRCH RNP showed similar results but with modestly lower efficiencies (FIGS. 30C-30E), confirming that RNP electroporation was less efficient than mRNA electroporation using these methods.

To determine whether ABE-mediated conversion of HBB^{S} to HBB^{S} alters erythropoiesis, flow cytometry was used to track the expression of cell-surface maturation markers CD49d, CD235a, and Band3 during the 18-day culture period^{34, 35} and used a Hoechst stain to distinguish enucleated reticulocytes from earlier-stage nucleated erythroblasts. No differences in the expression kinetics of these markers were observed between edited and unedited cells (FIG. 36), suggesting that editing with ABE8e-NRCH does not alter erythropoiesis.

### Genome-Wide Computational and Experimental Off-Target Analysis

To assess genomic off-target editing that might be introduced by ABE8e-NRCH and the HBB^{S}-targeting sgRNA, both computational and experimental methods were used to nominate candidate off-target sites genome-wide^{36, 37} (FIG. 30F). The Cas-OFFinder algorithm was used to computationally identify all 140 NRCH PAM-containing genomic sites with three or fewer mismatches to the protospacer, or two or fewer mismatches allowing for wobble base pairing. CIRCLE-seq, an experimental off-target identification method38, 39, was also performed to detect where Cas9-NRCH nuclease programmed with the HBB^{S}-targeting sgRNA cleaves purified human genomic DNA in vitro. CIRCLE-seq identified 601 candidate off-target sites. The 140 sites nominated by Cas-OFFinder and the 601 top sites nominated by CIRCLE-seq shared only 15 sites in common, consistent with previous reports of a low degree of overlap between computationally predicted and experimentally identified off-target loci^{40, 41}.

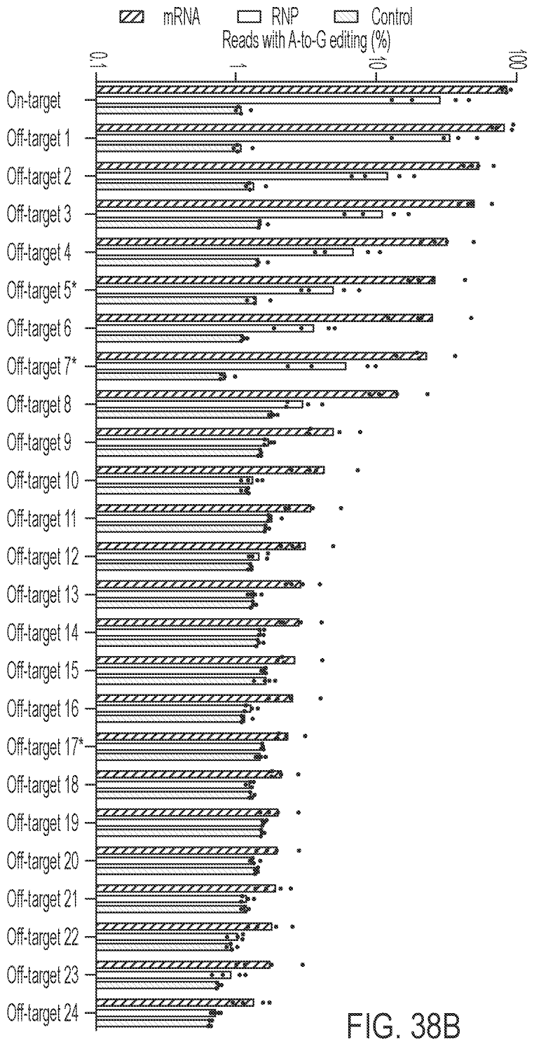

Next, off-target editing in SCD patient CD34+ HSPCs treated with ABE8e-NRCH mRNA or RNP was assessed. Multiplex targeted HTS were performed of 217 of the top-scoring potential off-target sites: 103 identified by CIRCLE seq and 129 identified by Cas-OFFinder, including 15 shared sites. Twelve sites identified by Cas-OFFinder were not amenable to multiplex sequencing. Point mutations consistent with adenine base editing were detected above that of untreated control cells at 24 of the 217 sequenced sites. All 24 sites at which off-target editing was observed were candidates identified by CIRCLE-seq, and four of them were also identified by Cas-OFFinder (FIGS. 30F, 38, 39), highlighting the importance of using experimental methods to define genome-wide off-target activity.

Off-target activity was distributed predominantly in intergenic and intronic regions, with the exception of one off-target point mutation found in the promoter region of CCDC85B (344 base pairs upstream of the transcription start site) and two found in exons (FIG. 30G). Only silent point mutations were introduced by off-target base editing in the two exons, which included HBD, a close homologue of the target HBB gene, and SLC34A1, a kidney-specific gene.

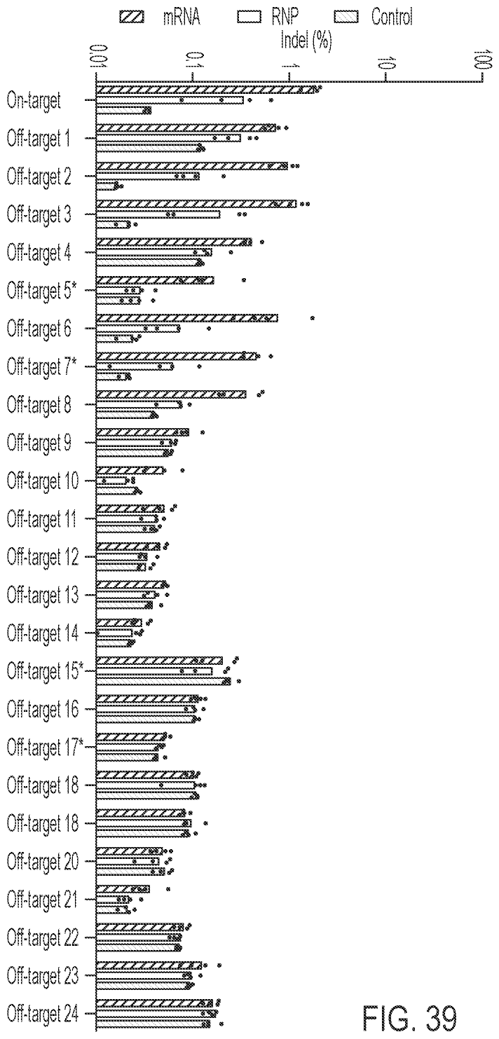

Off-target editing was less frequent after electroporation with ABE8e-NRCH RNP rather than mRNA, possibly due to shorter duration of exposure and/or lower editing activity (FIGS. 30H, 38, 39)^{2, 42, 43.} Indel frequencies were <2% at all off-target sites (FIG. 39). Collectively, these findings from genome-wide computational and experimental off-target analyses did not reveal any off-target point mutations anticipated to have clinical relevance to hematopoiesis from base editing of SCD patient HPSCs with ABE8e-NRCH.

Transplantation of Edited Human Cells into Immunodeficient Mice

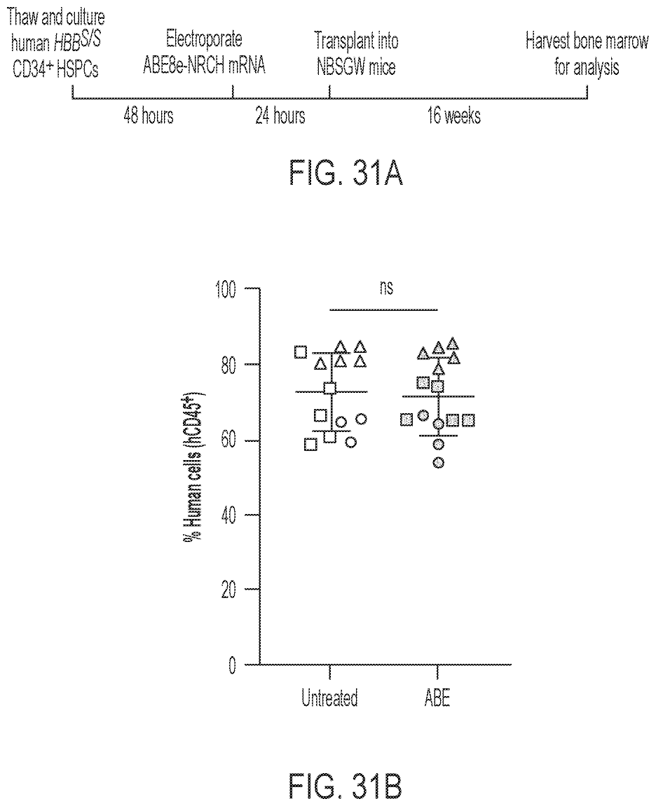

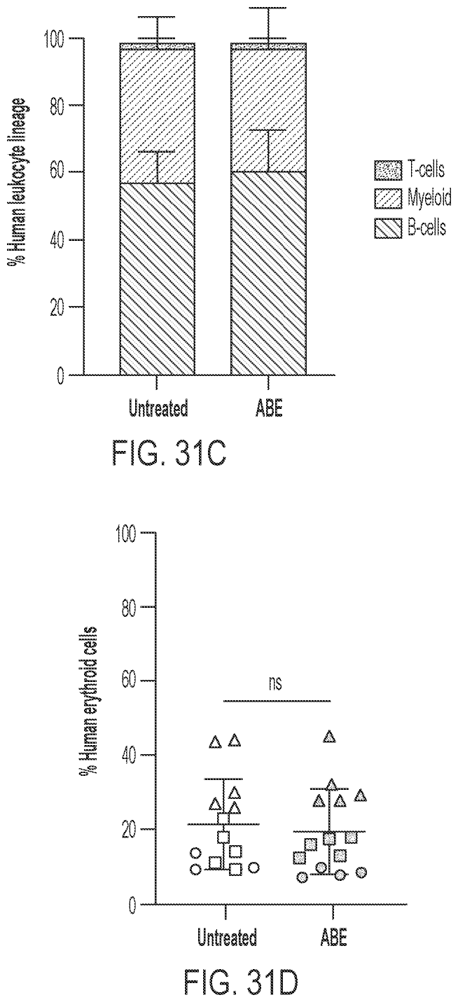

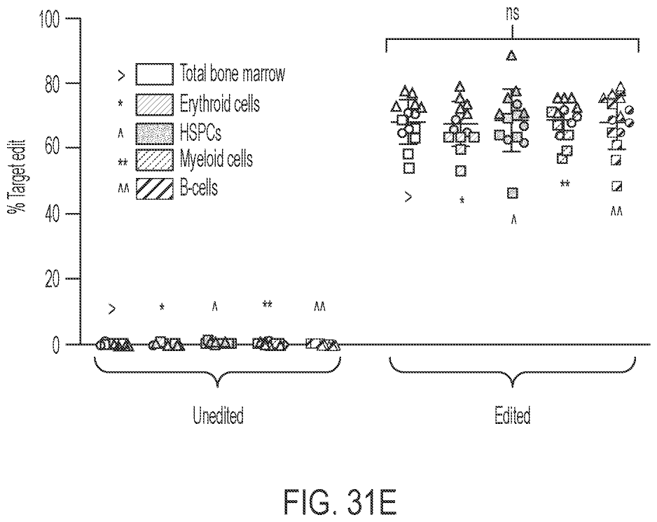

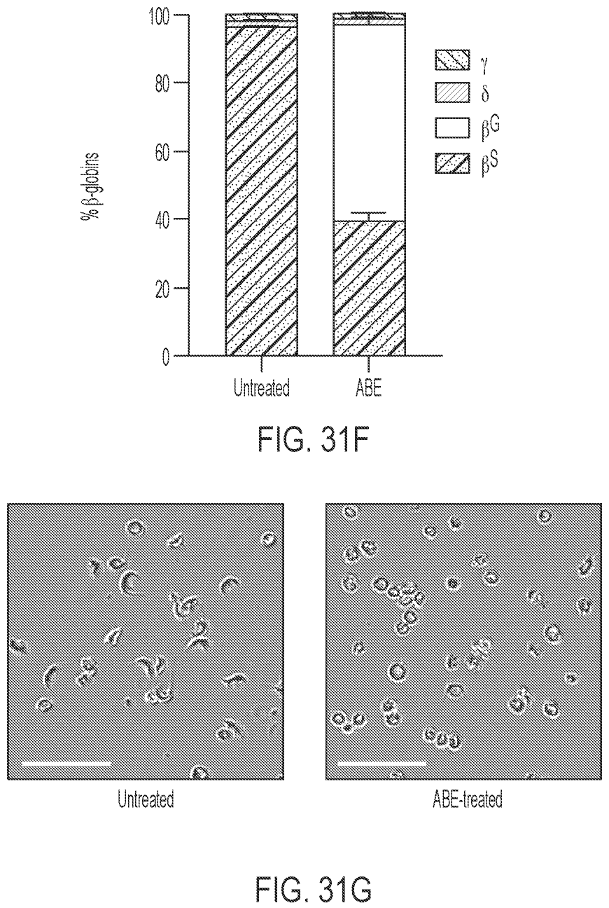

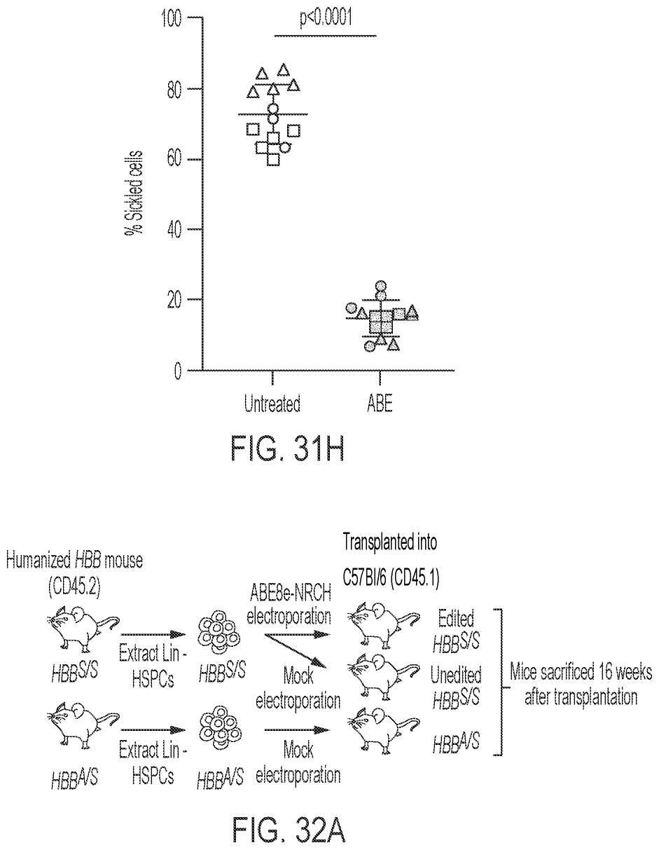

It was next examined whether base editing can convert HBB^{S} to HBB^{G} in transplanted HSCs that have repopulated bone marrow, without disrupting their multipotency, an important therapeutic requirement. CD34+ HSPCs from three different SCD patients were edited by electroporation of ABE8e-NRCH mRNA with sgRNA, or of ABE8e-NRCH RNP. After 24 hours, the resulting six sets of cells and a set of unedited control cells from each donor were each transplanted via tail-vein injection into 3-4 immunodeficient NOD, B6.SCID Il2rγ−/−KitW41/W41 (NBSGW) mice, each 7-9 weeks old. Sixteen weeks after infusion, the mice were sacrificed and their bone marrow was harvested for analysis (FIG. 31A). By this time, remaining human donor-derived cells are thought to be generated from long-term bone marrow repopulating HSCs capable of sustaining a hematopoietic system¹⁵.

The disruption of targeted genes in HSCs or T-cells cells through the introduction of DSBs or by Cre recombinase-mediated deletion has been reported to produce defects in engraftment or memory T-cell maintenance^{25, 44}. To determine whether ABE8e-NRCH conversion of HBB^{S} to HBB^{G} causes defects in differentiation potential or survival of detectable lineages, the human hematopoietic lineages present in recipient mouse bone marrow were assessed after transplantation. Flow cytometry using an anti-human CD45 antibody revealed approximately 70% engraftment of ABE8e-NRCH edited or untreated control CD34+ cells (FIG. 31B). Flow cytometry to quantify the relative abundances of human B-cells (CD19+), myeloid cells (CD33+), T-cells (CD3+), and erythroid cells (CD235a+) revealed equivalent proportions of each lineage in animals receiving either unedited or edited cells (FIGS. 31C, 31D, Table 5). Together, these results indicate that the differentiation potential of CD34+ cells was not altered by base editing.

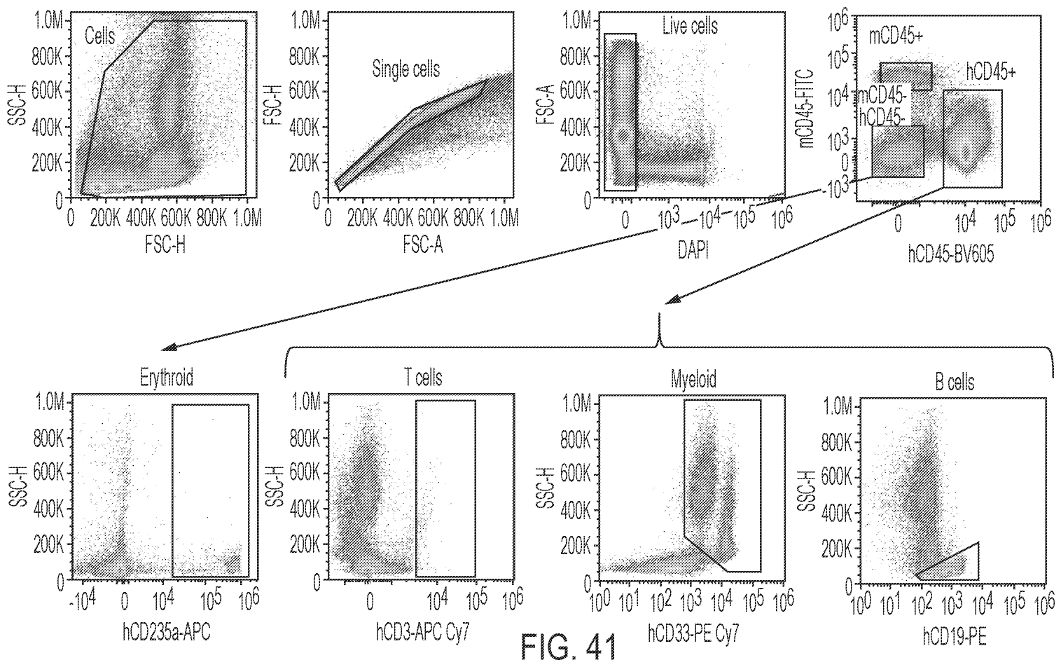

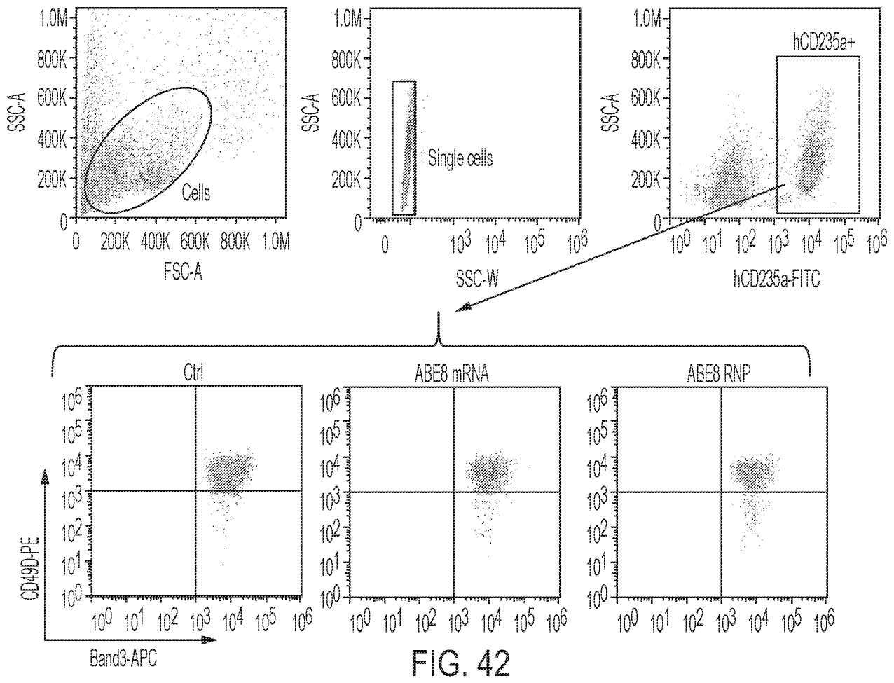

Skewed hematopoiesis resulting from base editing could be reflected by alterations in editing levels within affected lineages. To examine this possibility, frequencies of edited alleles each lineage that could be readily purified for DNA sequencing were assessed. Fluorescence activated cell sorting (FACS) and human lineage-specific fluorescent antibodies were used to purify donor-derived mononuclear cells (total bone marrow; CD45+), B-cells (CD19+), myeloid cells (CD33+), HSPCs (CD34+) and erythroblasts (CD235a+) from mouse recipient bone marrow (FIGS. 41-42). PCR amplification of extracted genomic DNA followed by HTS of the targeted region of the HBB locus revealed that all isolated cell populations contained the desired HBB^{S}-to-HBB^{G} edit at similar frequencies ranging from 67.7±6.6% to 69.0±5.7% (FIG. 31E), suggesting that allele proportion was maintained in HSCs and in their various differentiated progeny. The modest decrease from 80% editing observed before transplantation could reflect slightly higher editing efficiency of non-repopulating cells within the complex mixture of CD34+ HSPCs, with 68% editing efficiency achieved in repopulating HSCs. Collectively, these results indicate that ABE8e-NRCH mediates efficient conversion of HBB^{S} to HBB^{G} in repopulating HSCs without impeding their engraftment or multipotency relative to untreated controls.

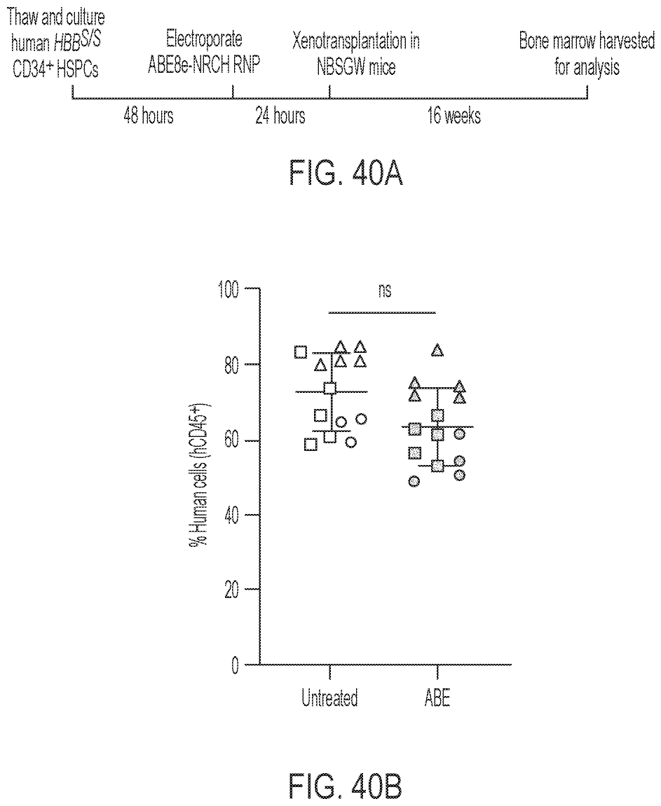

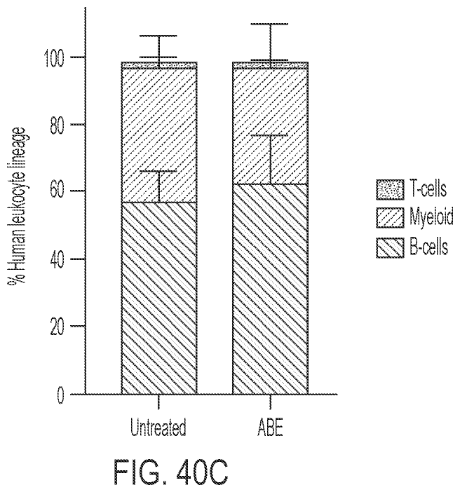

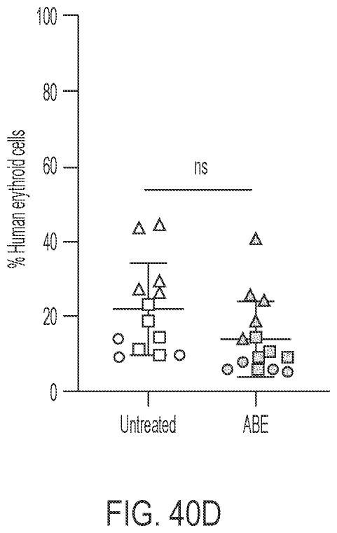

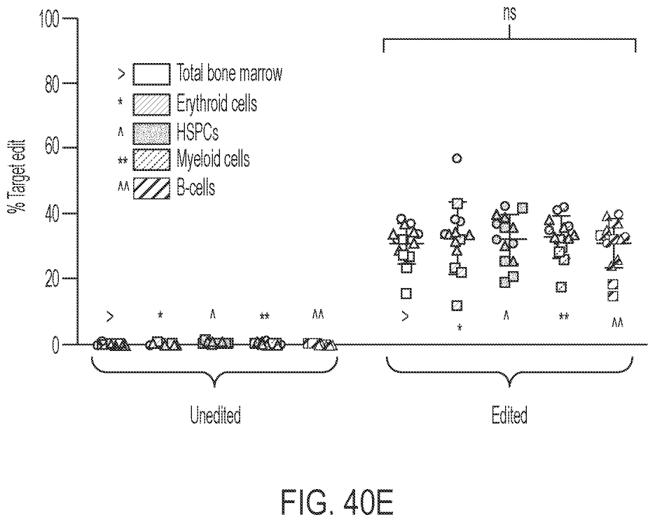

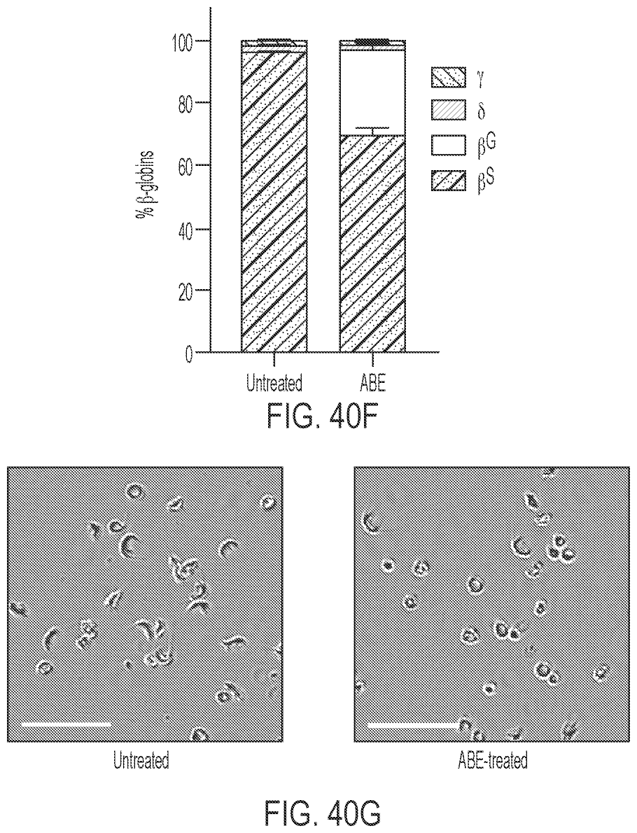

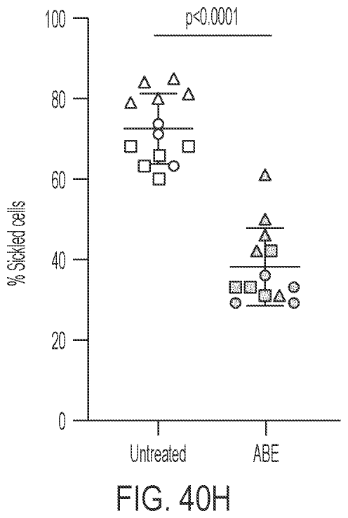

Circulating human RBCs are very short-lived in mice¹⁵. Therefore, the properties of human erythroid cells isolated from the bone marrow of mice transplanted with ABE-treated or untreated SCD patient CD34+ cells were examined. Human donor cell-derived CD235a+ erythroblasts were purified by magnetic-activated cell sorting (MACS) and analyzed for β-globin protein content by reverse-phase HPLC. The fraction of βS dropped from 96±0.28% of total β-like globin in erythroid cells derived from unedited HSPCs to 40±2.3% after base editing. The PG fraction was undetectable in erythroid cells from unedited HSPCs and rose to 58±2.8% after base editing (FIG. 31F). Human erythroid cells derived from edited HSPCs showed a 5-fold reduction in sickling compared to unedited control cells (14.5±5.1% vs. 72.5±8.6%; FIGS. 31G-31H). Cells edited using RNP showed similar trends, although editing was less efficient (FIG. 40). Taken together, these data indicate that HBB^{S}-to-HBB^{G} base-edited SCD donor-derived HSCs can repopulate the hematopoietic system normally and generate erythroid cells containing mostly PG with a greatly reduced propensity for hypoxic sickling.

### Transplantation of Edited Mouse HSCs in a SCD Mouse Model

Transplantation of human HSPCs into mice cannot be used to assess physiological SCD disease phenotypes due to the short lifetime of circulating human red blood cells in mice. To determine if base editor conversion of HBB^{S} to HBB^{G} can alleviate hematological phenotypes of SCD, lineage-negative (Lin−) HSPCs were edited from a mouse SCD model (also known as the “Townes mouse”) in which the endogenous adult-expressed α- and β-like globin genes are replaced by human globin genes, with the majority of β-like globin expression arising from human HBB^{S} and leading to sickle cell disease phenotypes32. Mice harboring one normal and one SCD HBB allele (genotype HBB^{A/S}) model the human SCD heterozygotes (sickle cell trait), a generally healthy condition (Table 6) in this animal model and in humans by most metrics.

TABLE 6
Complete blood counts from SCD model mice. Complete blood counts were
collected from transplanted animals using a FORCYTE Veterinary Hematology
Analyzer 16 weeks after the transplant before sacrifice. Generally healthy
HBB^{A/S} untransplanted animals were also measured at 4-6 months of
age. RBC, red blood cells; Hb, hemoglobin; Hct, hematocrit; MCV, mean
corpuscular volume; MCH, mean corpuscular hemoglobin; MCHC, mean corpuscular
hemoglobin concentration; RDW, red cell distribution width; WBC, white blood
counts. Data are shown as mean values ± SD. Statistical significance
was assessed using one-way ANOVA, with Sidak's multiple comparisons test
of the edited HBB^{S/S} values in comparison to each other group to calculate
p-values.

| Condition | Unedited | Edited | Unedited | Unedited |
|---|---|---|---|---|
| Genotype | HBB^{S/S} | HBB^{S/S} | HBB^{A/S} | HBB^{A/S} |
| Transplanted | Yes | Yes | Yes | No |
| No. of | 6 | 6 | 2 | 5 |
| mice analyzed | | | | |
| RBC (×10⁶/μL) | 6.52 ± 0.37 | 10.37 ± 0.81 | 10.74 ± 0.04 | 10.46 ± 0.60 |
| | ****p < 0.0001 | | ns, p = 0.8402 | ns, p = 0.9916 |
| Reticulocytes (%) | 41.45 ± 5.69 | 13.72 ± 5.81 | 9.46 ± 5.44 | 8.80 ± 1.46 |
| | ****p < 0.0001 | | ns, p = 0.6700 | ns, p = 0.3239 |
| Hb (g/dL) | 8.67 ± 0.42 | 10.77 ± 0.41 | 10.80 ± 0.71 | 10.88 ± 0.34 |
| | ****p < 0.0001 | | ns, p = 0.9996 | ns, p = 0.9624 |
| Hct (%) | 25.33 ± 1.27 | 30.70 ± 2.82 | 31.35 ± 0.35 | 32.70 ± 1.71 |
| | ***p = 0.0009 | | ns, p = 0.9717 | ns, p = 0.3143 |
| MCV (fL) | 38.87 ± 0.83 | 29.62 ± 1.02 | 29.15 ± 0.21 | 31.28 ± 1.32 |
| | ****p < 0.0001 | | ns, p = 0.9278 | *p = 0.0493 |
| MCH (pg) | 12.72 ± 1.74 | 10.40 ± 0.71 | 11.90 ± 1.98 | 10.40 ± 0.37 |
| | *p = 0.0144 | | ns, p = 0.3884 | ns, p > 0.9999 |
| MCHC (g/dL) | 33.98 ± 1.65 | 35.22 ± 2.44 | 35.20 ± 0.85 | 33.30 ± 0.88 |
| | ns, p = 0.5726 | | ns, p > 0.9999 | ns, p = 0.2571 |
| RDW (%) | 35.72 ± 1.07 | 27.08 ± 1.93 | 23.65 ± 1.06 | 23.70 ± 0.44 |
| | ****p < 0.0001 | | *p = 0.0182 | **p = 0.0022 |
| WBC | 39.67 ± 7.36 | 27.70 ± 6.99 | 26.49 ± 10.17 | 23.51 ± 6.88 |
| (×10³/uL) | *p = 0.0379 | | ns, p = 0.9961 | ns, p = 0.7390 |
| Platelets | 558.2 ± 95.4 | 614.8 ± 138.7 | 732.5 ± 178.9 | 1023.6 ± 134.1 |
| (×10³/uL) | ns, p = 0.8381 | | ns, p = 0.6233 | ***p = 0.0003 |

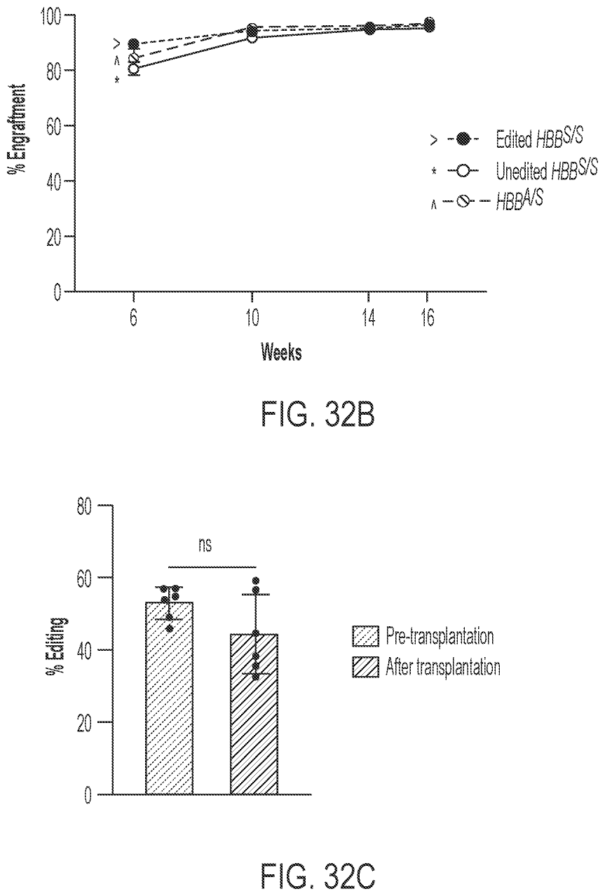

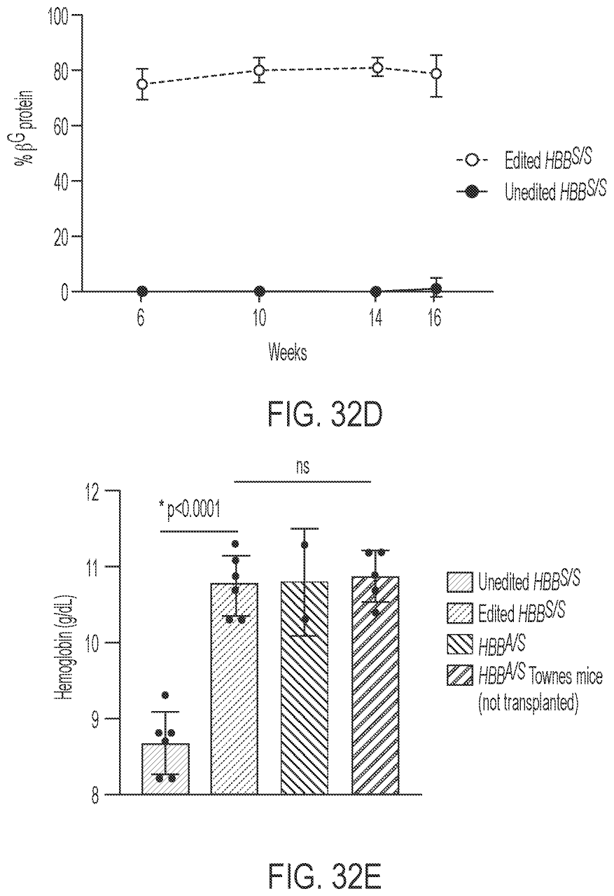

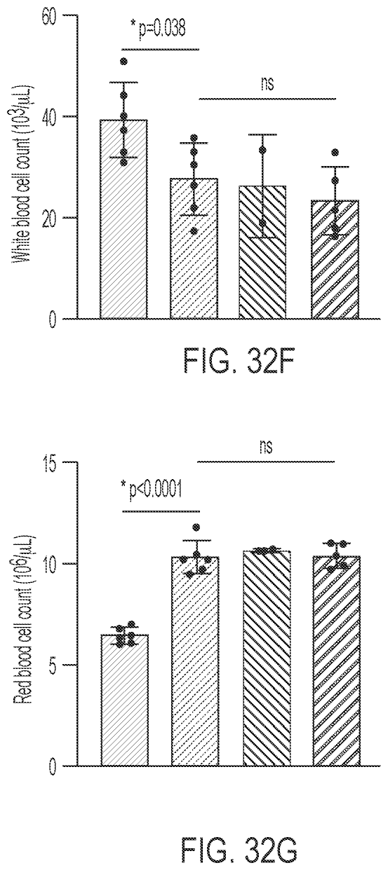

ABE8e-NRCH RNP was electroporated into HBB^{S/S} HSPCs from Townes mice and transplanted edited HSPCs into irradiated adult recipient mice (8-12 weeks old) at 24 hours after electroporation. Unedited HBB^{S/S} and HBBA/S HSPCs were used as disease and healthy controls, respectively. Donor mice hematopoietic cells express the CD45.2 antigen, while the congenic recipients express CD45.1, which can be discriminated from CD45.2 by flow cytometry using specific antibodies (FIG. 32A and Table 5)³². Blood was collected at 6, 10, 14, and 16 weeks after transplantation to track engraftment and β-globin content.

By 10 weeks post-transplantation, donor engraftment was >90% in all mice as determined by expression of the CD45.2 surface marker on blood mononuclear cells (FIG. 32B). High-level engraftment of edited and control donor HSPCs persisted similarly throughout the time course, indicating that editing did not disrupt the fitness of transplanted HSCs. The frequency of HBB^{S}-to-HBB^{G} editing was measured by HTS of genomic DNA (FIG. 32C). Pre-transplantation HBB^{S}-to-HBB^{G} editing efficiency measured 3 days after electroporation in a subset of cells maintained in culture was 53±4.5%. Editing levels in genomic DNA extracted from whole blood after animals were sacrificed at 16 weeks post-transplantation showed 44±11% HBB^{G} allele frequency. As observed with human HSPCs (FIG. 31E), the modest decrease in HBB^{G} allele frequency during the 16 weeks of engraftment could arise if repopulating HSCs are less amenable to electroporation or base editing than short-term or non-repopulating cells within the heterogenous Lin− population.

To measure the effect of base editing on the hemoglobin composition of circulating RBCs, reverse-phase HPLC was performed on peripheral blood cell lysates from each time point. PG made up 70-85% of total β-like globin in blood from mice receiving edited SCD HSPCs, with little fluctuation throughout the course of the experiment (FIG. 32D). The enrichment of PG over the observed editing efficiency, 70-85% vs. 42-57%, respectively, likely arises from the increased lifetime of βG-expressing RBCs caused by a reduction in sickling.

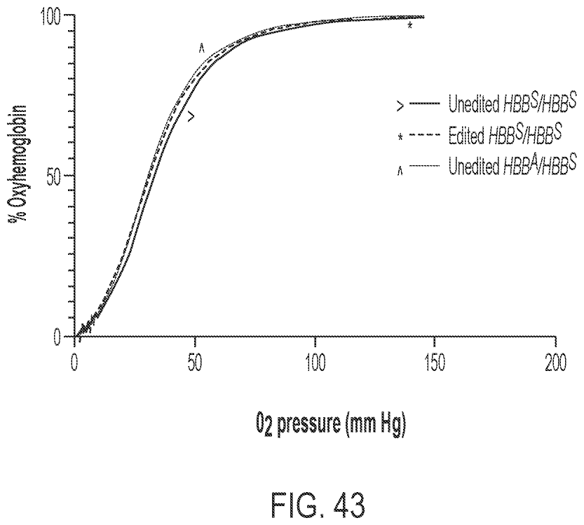

To determine whether PG leads to any defect in oxygen binding, hemoglobin-oxygen association was spectroscopically assessed in blood extracted from mice receiving unedited HBB^{S/S} cells, HBB^{A/S} cells, or edited HBB^{S/S} cells 14 weeks after transplantation (FIG. 43). No apparent difference was observed in oxygen binding, indicating that (3G-containing hemoglobin tetramers bind oxygen normally.

### Phenotypic Rescue of SCD in Transplanted Animals

Complete blood counts were performed on mice transplanted with edited (n=6) or unedited (n=6) mouse HBB^{S/S} HSPCs, as well as on two mice transplanted with unedited HBBA/S cells and five non-transplanted mice with an HBS^{A/S} genotype (FIGS. 32E-32H and Table 6). Compared to healthy controls (non-transplanted HBBA/S mice or those transplanted with HBB^{A/S} HSPCs), mice that received unedited mouse HBB^{S/S} HSPCs exhibited significant differences in RBC parameters, including decreased RBC count, decreased total blood hemoglobin concentration (Hb), elevated reticulocyte count, and elevated white blood cell count (FIGS. 32E-32H). These abnormalities are consistent with hemolytic anemia and also observed in SCD patients³². Importantly, base editing of HBB^{S/S} HSPCs restored all tested blood parameters to levels that were similar to those of healthy controls (FIGS. 32E-32H). These results indicate that base editing of HBB^{S} to HBB^{G} ex vivo followed by bone marrow transplantation can rescue the hematological defects caused by SCD.

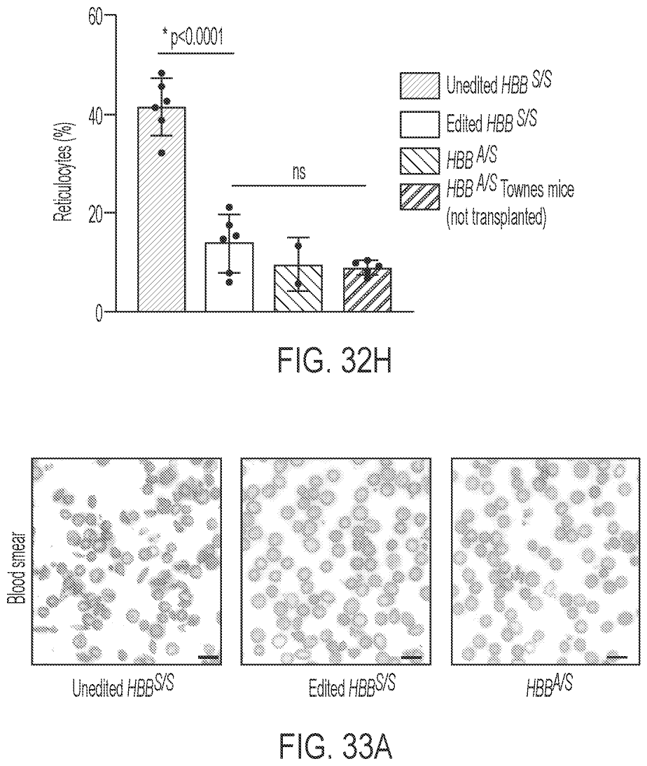

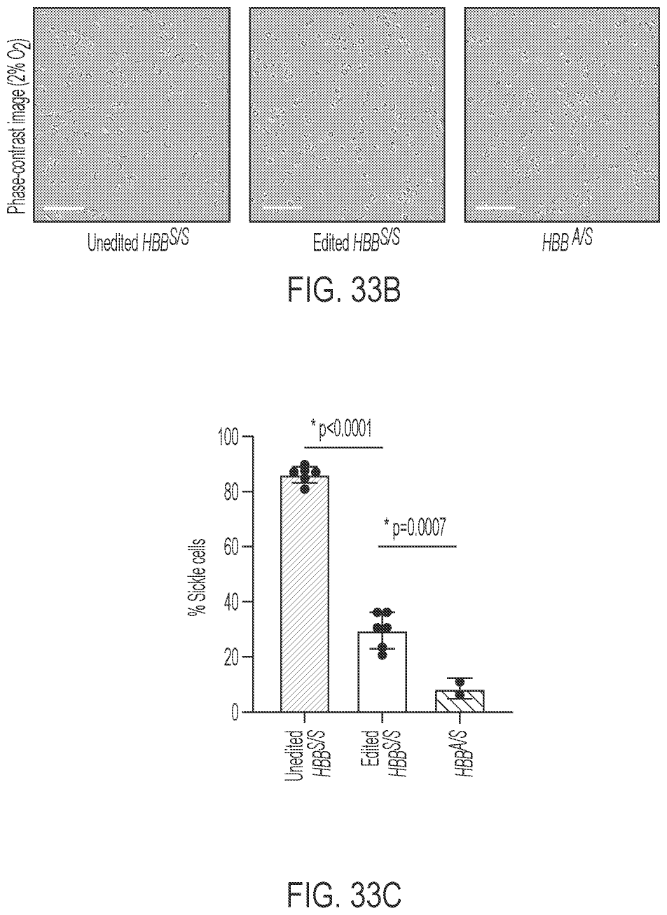

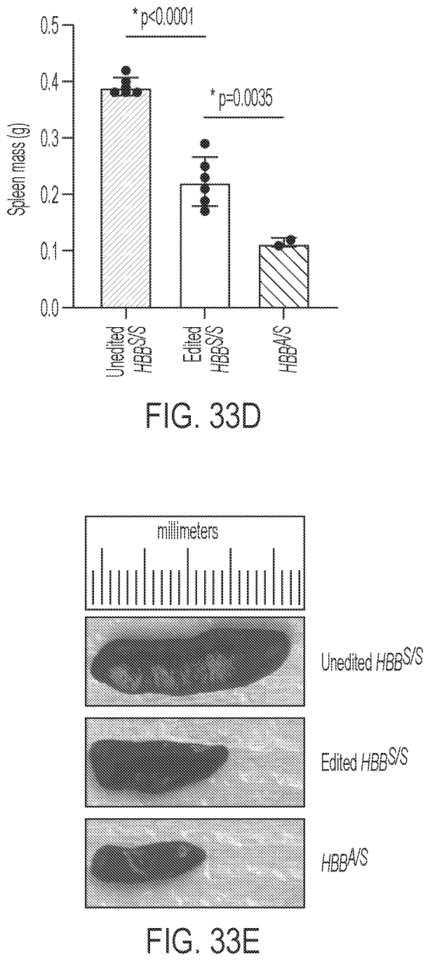

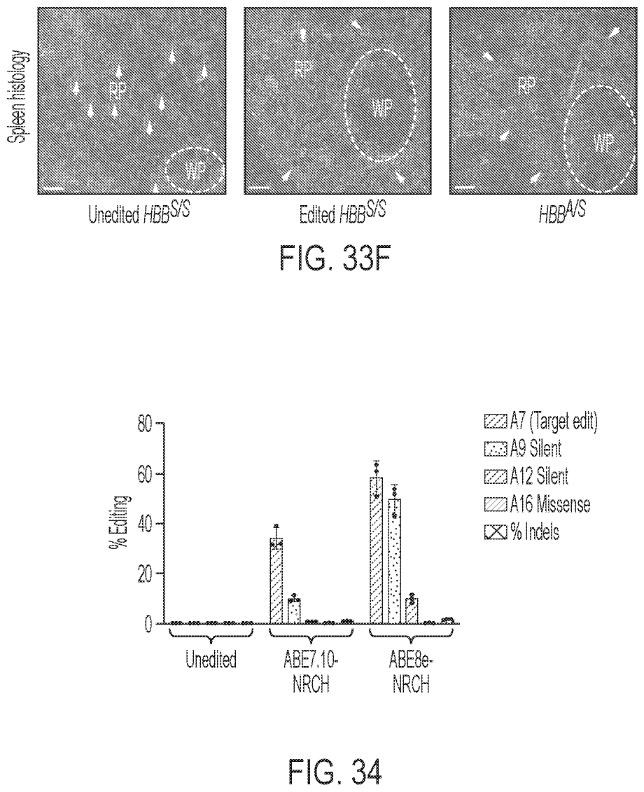

To assess the consequence of base editing on circulating RBC morphology, blood smears from mice at 16 weeks were analyzed after transplantation (FIG. 33A). Morphological abnormalities consistent with SCD were apparent in RBCs of mice that received unedited HBB^{S/S} cells. These included abundant oblong sickle forms, polychromasia (dark purple cells) reflecting reticulocytosis, and fragmentation (schistocytes). The RBCs of mice that were transplanted with HBB^{S}-to-HBB^{G} edited HSPCs showed a reduction in all pathologic morphologies and were more similar to RBCs of the healthy HBB^{A/S} control (FIG. 33A). Separately, blood was incubated for 8 hours in 2% oxygen to further induce RBC sickling (FIGS. 33B-33C). Red blood cells from mice transplanted with edited HBB^{S/S} HSPCs showed 29.8±6.5% sickling, compared to 86.3±3.0% of RBCs from mice receiving unedited HBB^{S/S} HSPCs, a 2.9-fold decrease. These data establish that transplantation of edited HBB^{S/S} HSPCs leads to durable production of PG-expressing RBCs that are resistant to sickling both in vitro after exposure to hypoxia and in vivo.

Splenomegaly (enlarged spleen) in SCD patients and mouse models results from phagocytosis of sickled cells, vascular congestion, and extramedullary erythropoiesis to compensate for accelerated RBC loss from hemolysis³². The average spleen mass of mice receiving edited HBB^{S/S} cells was 0.22±0.043g, compared to 0.39±0.016g in mice receiving unedited HBB^{S/S} cells and 0.11±0.007g in mice receiving HBB^{A/S} cells (FIGS. 33D-33E). Thus, the average spleen mass was restored by 61% towards that of healthy control mice in animals receiving base edited cells (FIG. 33D). Histopathology analysis of spleen from mice that received unedited HBB^{S/S} cells revealed RBC pooling caused by vascular congestion and increased ratio of red pulp to white pulp signifying extramedullary erythropoiesis. In contrast, the spleens of mice that received edited HBB^{S/S} cells resembled those of the healthy control mice that received HBB^{A/S} cells (FIG. 33F). Taken together, the persistence of ABE8e-NRCH-mediated HBB^{S}-to-HBB^{G} editing in bone marrow-repopulating HSCs and the partial or complete rescue of every examined SCD phenotype suggest that ex vivo base editing of HBB^{S} has the potential to alleviate SCD.

### Discussion

Adenine base editing enables precise and efficient conversion of A•T base pairs to GC without requiring double-strand DNA breaks, thereby circumventing uncontrolled and often undesired editing outcomes such as indels or translocations. An ABE variant with a laboratory-evolved ABE8e deaminase and a laboratory-evolved Cas9-NRCH domain that can directly convert the major SCD mutation (HBB^{S}) to a benign 3-globin gene variant (HBB^{G}) is described herein. This base editing strategy occurred efficiently (up to 80% editing in CD34+ HSPCs), led to low levels of indels (<3%), and restored functional hemoglobin without disruption of natural gene regulation or hematopoiesis.

The HBB^{A}-to-HBB^{S} mutation converts glutamic acid to valine at residue 6 of the (3-globin protein. Polymerization of HbS is mediated in part by interaction of the mutant Val6 with a hydrophobic pocked formed by amino acids Leu88, Phe85, Thr84, Glu73 and Ala70 of another 3-globin molecu1e45. The replacement of Val6 with Ala through conversion of HBB^{S} to HBB^{G} produces a profound reduction in RBC sickling, likely because the Ala6 side chain in HbG does not have sufficient affinity for the hydrophobic pocket of 3-globin, and is therefore incapable of supporting polymerization⁴⁶.

Numerous approaches for autologous genetic therapies to treat SCD are being evaluated in the clinic, including induction of γ-globin by genome editing or lentiviral vector modulation, lentiviral vector gene therapy to deliver an ectopic non-sickling 3-globin gene, and Cas9 nuclease-mediated homology directed repair (HDR) to correct the SCD mutation⁷⁻¹¹. It is not yet known which strategy is safest and most effective. However, the base editing approach demonstrated here offers several potential advantages.

First, elimination of the disease-causing mutation by precise conversion of HBB^{S} to HBB^{G} may reduce the RBC HbS concentration (the primary determinant of polymerization) more effectively than lentiviral vector-mediated β-like globin expression or induction of HbF where HBB^{S} alleles remain intact. While genome editing or gene delivery in HSPCs has been shown to repress PS in erythroid progeny by 30-70%^{34, 47-49}, up to 80% decreased levels of PS was achieved in bulk erythroid populations by direct base editing of HBB^{S}. Therapeutic thresholds for HbF induction that are required to entirely prevent SCD-associated complications are not well defined. In contrast, allogeneic bone marrow transplantation studies of patients with SCD have shown that approximately 25-30% chimerism of non-sickling cells (HBB^{NA} or HBB^{A/S}) induce substantial clinical improvement^{50, 51}. The results in this study suggest that an even greater proportion of chimerism for HBB^{G} should be attainable using the approach described here.

Second, base editing largely avoids double-stranded DNA breaks (DSBs) that are generated by Cas9 nuclease, as well as insertional mutagenesis that can be caused by integration of DNA vectors required by some alternative approaches⁵²⁻⁵⁴. Potentially undesired outcomes of DSBs include uncontrolled mixtures of indels⁵⁵, translocations⁵⁶, chromosomal loss¹⁸, large deletions that are difficult to detect by HTS¹⁹⁻²¹, and selection against the p53 DNA damage response that suppresses tumor formation²²⁻²⁵.

Third, base editing obviates the requirement for co-delivery of a donor DNA template that is necessary for HDR or of viral DNA for gene therapy. The introduction of DNA can lead to toxicity and undesired genomic integration. In contrast, base editing directly converts pathogenic HBB^{S} into the naturally occurring non-pathogenic HBB^{G} allele that is known to be benign, even in homozygous or hemizygous form, with no DNA delivery requirement, as demonstrated by the mRNA and RNP treatments in this study.

Potential undesired consequences of base editing HSPCs were examined. Base editors can cause bystander editing of nearby adenine (ABE) or cytidine (CBE) nucleotides. In this study, bystander editing was limited to synonymous changes that should not interfere with the intended editing outcome. Spurious editing of RNA can occur but is transient when base editor mRNA or RNP is used, and did not appear to affect repopulation or differentiation of HSCs in this study. Indeed, it has been shown that levels of spurious RNA editing by ABEs are low in hematopoietic cells⁵⁷. Off-target DNA editing can also occur. Of the 24 identified sites at which off-target editing was observed, no missense mutations were introduced and no off-target edits were detected that are predicted to disrupt the function of any regulatory regions. Nevertheless, further refinement of this strategy to minimize off-target editing, for example by testing alternate ABE variants shown to minimize Cas-dependent or Cas-independent off-target editing^{2, 27, 58, 59}, optimizing doses of editing agent^{2, 60}, or optimizing delivery methods, may enhance the safety and therapeutic potential of this approach.

The ex vivo delivery procedure used in this study is similar to methods currently used for HSC editing in clinical trials¹⁴. The ABEs were electroporated as mRNA or RNP to minimize the duration of exposure to the editing agent, thereby reducing off-target editing activity^{2, 61}. HSCs were edited using a single electroporation and transplanted into adult animals after 24 hours to minimize the duration of in vitro tissue culture that can lead to the loss of HSC engraftment and multipotency⁶². The base-edited SCD patient-derived CD34+ cells used in this study could be the basis for autologous therapies based on precise conversion of the SCD mutation to a benign variant ex vivo, followed by transplantation.

Ideally, in vivo delivery methods that support base editing in HSCs would enable the administration of this editing strategy by injection, rather than the more demanding current approach, which requires isolation of patient HSCs followed by their in vitro modification and transplantation. Recent developments in the delivery of genome editing agents in vivo are encouraging^{63-66.} Such methods applied to HSCs could simplify the procedure and greatly broaden the availability of a potential one-time treatment for SCD.

### Methods

### HEK293T Cell Culture and Editing

HEK293T cells (ATCC CRL-3216) were cultured in Dulbecco's modified Eagle's medium (Corning) supplemented with 10% fetal bovine serum (ThermoFisher Scientific) and maintained at 37° C. with 5% CO₂. For plasmid transfection, HEK293T cells were seeded at 20,000 cells per well on 96-well poly-D-lysine plates (Corning) in the same culture medium. Cells were transfected 24-30 h after plating with 0.5 μL Lipofectamine 2000 (ThermoFisher Scientific) using 200 ng base editor plasmid and 66 ng guide RNA plasmid following manufacturer's instructions. Cells were cultured for 3 days following lipofection, then washed with PBS (ThermoFisher Scientific). Genomic DNA was extracted after removing PBS by addition of 50 μL freshly prepared lysis buffer (10 mM Tris-HCl, pH 7.5, 0.05% SDS, 25 μg/mL proteinase K (ThermoFisher Scientific)) directly into each transfected well. The mixture was incubated at 37° C. for 1 h then heat inactivated at 80° C. for 30 min. One microlitre of this lysate was used as a PCR template for high-throughput sequencing.

### High-Throughput Sequencing of the HBB SCD Locus in HEK293T Cells

High-throughput sequencing (HTS) of genomic DNA was performed as previously described 26, 67. Primers for amplification of the HBB SCD locus in HEK293T cells were:

GAN162F
(SEQ ID NO: 492)
5′-ACACTCTTTCCCTACACGACGCTCTTCCGATCTNNNNAGGGTTGGCC
AATCTACTCCC-3′;
and
GAN163R
(SEQ ID NO: 493)
5′-TGGAGTTCAGACGTGTGCTCTTCCGATCTGTCTTCTCTGTCTCCACA
TGCC-3′.

Underlined sequences represent adapters for Illumina sequencing. Following Illumina barcoding, PCR products were pooled and purified by electrophoresis with a 2% agarose gel using a Monarch DNA Gel Extraction Kit (New England Biolabs), eluting with 30 μL H2O. DNA concentration was quantified with Qubit dsDNA High Sensitivity Assay Kit (ThermoFisher Scientific) and sequenced on an Illumina MiSeq instrument (single-end read, 250-300 cycles) according to the manufacturer's protocols. Alignment of fastq files and quantification of editing frequency was performed using CRISPResso2⁶⁸ in batch mode with a window width of 34 nucleotides.

ABE8e-NRCH mRNA

ABE8e-NRCH mRNA was transcribed in vitro from PCR product using full substitution of N1-methylpseudouridine for uridine. mRNA was capped co-transcriptionally using CleanCap AG analog (TriLink Biotechnologies) resulting in a 5′ Cap 1 structure. In vitro transcription reaction was performed as previously described 69 with the following changes; 16.5 mM magnesium acetate and 4 mM CleanCap AG were used as the final concentration during transcription, and mRNAs were purified using RNeasy kit (QIAgen). Mammalian-optimized UTR sequences (TriLink) and a 120 base poly A tail were included in the transcribed PCR product.

### ABE8e-NRCH Protein

ABE8e-NRCH protein was codon optimized for bacterial expression and cloned into the protein expression plasmid pD881-SR (Atum, Cat. No. FPB-27E-269). The expression plasmid was transformed into BL21 Star DE3 competent cells (ThermoFisher, Cat. No. C601003). Colonies were picked for overnight growth in terrific broth (TB)+25 ug/mL kanamycin at 37° C. The next day, 2 L of pre-warmed TB were inoculated with overnight culture at a starting OD₆₀₀ of 0.05. Cells were shaken at 37° C. for about 2.5 hours until the OD₆₀₀ was ˜1.5. Cultures were cold shocked in an ice-water slurry for 1 hour, following which L-rhamnose was added to a final concentration of 0.8% to induce. Cultures were then incubated at 18° C. with shaking for 24 hours to express protein. Following induction, cells were pelleted and flash-frozen in liquid nitrogen and stored at −80 degrees. The next day, cells were resuspended in 30 mL cold lysis buffer (1 M NaCl, 100 mM Tris-HCl pH 7.0, 5 mM TCEP, 10% glycerol, with 5 tablets of cOmplete, EDTA-free protease inhibitor cocktail tablets (Millipore Sigma, Cat. No. 4693132001). Cells were passed three times through a homogenizer (Avestin Emulsiflex-C3) at ˜18,000 psi to lyse. Cell debris was pelleted for 20 minutes using a 20,000 g centrifugation at 4° C. Supernatant was collected and spiked with 40 mM imidazole, followed by a 1-hour incubation at 4° C. with 1 mL of Ni-NTA resin slurry (G Bioscience Cat. No. 786-940, prewashed once with lysis buffer). Protein-bound resin was washed twice with 12 mL of lysis buffer in a gravity column at 4° C. Protein was eluted in 3 mL of elution buffer (300 mM imidazole, 500 mM NaCl, 100 mM Tris-HCl pH 7.0, 5 mM TCEP, 10% glycerol). Eluted protein was diluted in 40 mL of low-salt buffer (100 mM Tris-HCl, pH 7.0, 5 mM TCEP, 10% glycerol) just before loading into a 50 mL Akta Superloop for ion exchange purification on the Akta Pure25 FPLC. Ion exchange chromatography was conducted on a 5 mL GE Healthcare HiTrap SP HP pre-packed column (Cat. No. 17115201). After washing the column with low-salt buffer, the diluted protein was flowed through the column to bind. The column was then washed in 15 mL of low salt buffer before being subjected to an increasing gradient to a maximum of 80% high salt buffer (1 M NaCl, 100 mM Tris-HCl, pH 7.0, 5 mM TCEP, 10% glycerol) over the course of 50 mL, at a flow rate of 5 mL per minute. 1-mL fractions were collected during this ramp to high-salt buffer. Peaks were assessed by SDS-PAGE to identify fractions containing the desired protein, which were concentrated first using an Amicon Ultra 15-mL centrifugal filter (100-kDa cutoff, Cat. No. UFC910024), followed by a 0.5-mL 100-kDa cutoff Pierce concentrator (Cat. No. 88503). Concentrated protein was quantified using a BCA assay (ThermoFisher, Cat. No. 23227).

### Isolation and Culture of CD34+ Hematopoietic Stem and Progenitor Cells (HSPCs)

Circulating G-CSF-mobilized human mononuclear cells were obtained from deidentified healthy adult donors (Key Biologics, Lifeblood). Plerixafor-mobilized CD34+ cells from patients with SCD were collected according to the protocol “Peripheral Blood Stem Cell Collection for Sickle Cell Disease Patients” (ClinicalTrials.gov identifier NCT03226691), which was approved by the human subject research institutional review boards at the National Institutes of Health and St. Jude Children's Research Hospital. All participants provided informed consent. Enrichment of CD34+ cells was performed by immunomagnetic bead selection using a CliniMACS Plus or AutoMACS instrument (Miltenyi Biotec). CD34+ cells were maintained in stem cell culture media: X-VIVO-10 (Lonza, BEBP02-055Q) media supplemented with 100 ng/μL human SCF (R&D systems, 255-SC/CF), 100 ng/μL human TPO (R&D systems, 288-TP/CF) and 100 ng/μL human Flt-3 ligand (R&D systems, 308-FK/CF). Cells were seeded and maintained at a density of between 0.5-1×10⁶ cells/mL.

RNP and mRNA Electroporation in Human HSPCs

Electroporation was performed with an ATX MaxCyte electroporator using electroporation program HSC3. The modified synthetic sgRNA contained 2′-O-methyl modifications in the first three and last three nucleotides, and phosphorothioate bonds between the first three and last three nucleotides and was purchased from BioSpring. CD34+ HSPCs were thawed 48 hours before electroporation. mRNA and sgRNA were mixed at a 1:1 weight ratio prior to electroporation. RNPs were formed at a 1:1.5 ratio of ABE and Makassar sgRNA, and incubated for 20 minutes at room temperature prior to electroporation. mRNA/SgRNA were electroporated at 200 μg/mL of mRNA and RNP were electroporated at a final concentration of 9 μM of protein per reaction. 20-40 million cells/mL were electroporated in 100 μL of Maxcyte Buffer in OC-100 cartridges for transplantation into NBSGW animals. Electroporated cells were recovered in stem cell culture media composed of X-VIVO 10 media including cytokines (Flt-3 ligand, SCF, and TPO). Cells were maintained in culture at a density between 0.5-1×10⁶ per mL. Genomic DNA was extracted on culture days 2 and 6 using Qiagen extraction buffer then analyzed by HTS for editing efficiency.

### High-Throughput Sequencing of the HBB SCD Locus in Blood Cells

After editing, the HBB SCD locus was amplified from genomic DNA with oligonucleotide primers:

Forward.LF
(SEQ ID NO: 494)
5′- CTACACGACGCTCTTCCGATCTTGGCCAATCTACTCCCAGGAGCAG
G-3′;
and
Reverse.LR
(SEQ ID NO: 495)
5′- CAGACGTGTGCTCTTCCGATCTTCAAAGAACCTCTGGGTCCAAGGG
T-3′

Underlined sequences represent adapters for Illumina sequencing. Following Illumina barcoding, PCR products were pooled and HTS was conducted using a MiSeq or MiniSeq (Illumina). Sequences were analyzed by joining paired reads and analyzing amplicons for indels or the desired test sequence using CRIS.py70. Indels were reported as the number of reads without the WT amplicon length.

### Erythroid Cell Culture

Erythroid differentiation of CD34+ cells was performed using a 3-phase protocol34, 35. Phase 1 (days 1-7): IMDM (Thermo Fisher Scientific, 12440061) with 2% human blood type AB plasma (SeraCare, 1810-0001), 3% human AB serum (Atlanta Biologicals, 540110) 1% penicillin/streptomycin (Thermo Fisher Scientific, 15070063), 3 units/mL heparin (Sagent Pharmaceuticals, NDC #25021-401-02), 3 units/mL EPO (Amgen, EPOGEN NDC #55513-144-01), 200 μg/mL holo-transferrin (Millipore Sigma, T0665, 10 ng/mL human SCF (R&D systems, 255-SC/CF), and 1 ng/mL human interleukin IL-3 (R&D systems, 203-IL/CF). Phase 2 (days 8-14): Phase 1 medium without IL-3. Phase 3 (days 15-21): Phase 2 medium without SCF and with holo-transferrin concentration increased to 1 mg/mL. Cells were maintained daily at a density of 0.1×10⁶/mL (phase 1), 0.2×10⁶/mL (phase 2) and 1.0×10⁶/mL (phase 3)

Erythroblast maturation was monitored by immuno-flow cytometry for the cell surface markers CD235a, CD49d, and Band3 (Table 5). To quantify erythroblast enucleation, 1.5-5×10⁵ CD34+-derived erythroid cells were incubated with Hoechst 33342 for 20 min at 37° C., fixed with 0.05% glutaraldehyde, and permeabilized with 0.1% Triton X-100. Cells were stained with FITC mouse anti-human CD235a then analyzed by flow cytometry.

### Hemoglobin Quantification

High-performance liquid chromatography (HPLC) quantification of individual globin chains was performed using reverse-phase columns on a Prominence HPLC System (Shimadzu Corporation). The eluted proteins were identified by light absorbance at 220 and 415 nm using a diode array detector. For analysis of erythroid cells derived from in vitro differentiation of human CD34+ cells, the relative amounts of different β-like globin proteins were calculated from the area under the 415-nm peak and normalized according to the DMSO control. They are expressed as a fraction of the total β-like globins including normal β (βA) sickle β (βS), Makassar β (βG), γ, and δ-globin.

### In Vitro Sickling Assay

Erythroid cells were incubated with Hoechst 33342 for 20 mins at 37oc, Hoechst negative population were sorted using a SH1800 (Sony Biotechnologies). Sorted cells (0.5-1.0×10⁵ cells) were seeded 12 or 96 well plate with 1 mL or 0.1 mL of phase 3 ED media under hypoxic conditions (2% 02) for 24 h. The IncuCyte® S3 Live-Cell Analysis System (Sartorius) with a 20× objective was used to monitor cell sickling, with images being captured every 1 h. The percentage of sickling was measured by manual counting of sickled cells versus normal cells based on morphology. For mouse transplantation studies, mouse blood was diluted (1:5000) in RPMI media and seeded in 6 well plate with 3 mL of RPMI media before imaging in IncuCyte S3.

### CIRCLE-Seq Off-Target Editing Analysis

Genomic DNA from HEK293T cells was isolated using Gentra Puregene Kit (QIAGEN) according to manufacturer's instructions. CIRCLE-seq was performed as previously described38, 39. Briefly, purified genomic DNA was sheared with a Covaris S2 instrument to an average length of 300 bp. The fragmented DNA was end repaired, A tailed and ligated to a uracil-containing stem-loop adaptor, using KAPA HTP Library Preparation Kit, PCR Free (KAPA Biosystems). Adaptor ligated DNA was treated with Lambda Exonuclease (NEB) and E. coli Exonuclease I (NEB) and then with USER enzyme (NEB) and T4 polynucleotide kinase (NEB). Intramolecular circularization of the DNA was performed with T4 DNA ligase (NEB) and residual linear DNA was degraded by Plasmid-Safe ATP-dependent DNase (Lucigen). In vitro cleavage reactions were performed with 250 ng of Plasmid-Safe-treated circularized DNA, 90 nM of Cas9-NRCH protein, Cas9 nuclease buffer (NEB) and 90 nM of synthetic chemically modified sgRNA (BioSpring), in a 100 μl volume. Cleaved products were A tailed, ligated with a hairpin adaptor (NEB), treated with USER enzyme (NEB) and amplified by PCR with barcoded universal primers NEBNext Multiplex Oligos for Illumina (NEB), using Kapa HiFi Polymerase (KAPA Biosystems). Libraries were sequenced with 150 bp paired-end reads on an Illumina MiSeq instrument. CIRCLE-seq data analyses were performed using open-source CIRCLE-seq analysis software and default recommended parameters (https://github.comitsailabSJ/circleseq).

### CasOFFinder Off-Target Editing Analysis

Computational prediction of NRCH PAM-containing potential off-target sites with minimal mismatches relative to the intended target site (three or fewer mismatches overall, or two or fewer mismatches allowing G:U wobble base pairings with the guide RNA) was performed using CasOFFinder^{36, 37}.

Targeted Amplicon Sequencing by rhAmpSeq

On- and off-target sites identified by CIRCLE-seq and CasOFFinder were amplified from genomic from CD34+ donors using rhAMPSeq system (IDT). Sequencing libraries were generated according to manufacturer's instructions and sequenced with 151-bp paired-end reads on an Illumina NextSeq instrument.

### Quantification of Base Editing Efficiency at Evaluated Off-Target Sites

The A•T-to G•C editing frequency for each position in the protospacer was quantified using CRISPRessoPooled (v2.0.41) with quantification_window_size 10, quantification_window_center −10, base_editor_output, conversion_nuc_from A, conversion_nuc_to G. The genomic features of the confirmed off-target sites were annotated using HOMER (v4.10) 71. The editing frequency for each site was calculated as the ratio between the number of reads containing the edited base (i.e., G) and the total number of reads. To calculate statistical significance of off-target editing for the ABE8e-NRCH mRNA or RNP treatments compared to control samples, a Chi-square test was applied for each of four samples (i.e., two donors, each with two replicates). The 2×2 contingency table was constructed based on the number of edited reads and the number of unedited reads in treated and control groups. FDR was calculated using the Benjamini/Hochberg method. The 24 reported significant off-targets were called based on: (1) FDR <0.05 and (2) difference in editing frequency between treated and control >1% for at least one treatment. The software used to conduct this analysis is available to download from this website: https://github.com/tsailabSJ/MKSR_off_targets.

### Ethical Approval for Studies Involving Mice

All studies utilizing mice were approved by the St. Jude Children's Research Hospital Institutional Animal Care and Use Committee under Protocol 579 entitled “Genetic Models for the Study of Hematopoiesis”. Mice were maintained in the St. Jude Children's Research Hospital Animal Resource Center according to recommendations in the Guide for the Care and Use of Laboratory Animals of the National Institutes of Health.

Transplantation of Gene-Edited CD34+ HSPCs into NOD.Cg-KitW-41J Tyr+ Prkdcscid Il2rgunlVVjl/Thomj (NBSGW) Mice

NBSGW mice were purchased from The Jackson Laboratory (stock no. 026622). Base edited or control CD34+ cells were administered at a dose of 0.2×106 per mouse with an IP injection of 10 mg/kg of busulfan (Busulfex; PDL BioPharm) 48 hours before infusion72 or 0.5×106 per mouse with no busulfan preconditioning by tail-vein injection in female mice aged 7-9 weeks. Chimerism post-transplantation was evaluated at 16-17 weeks in the bone marrow at the time of euthanasia (see supplemental Table 1). Cell lineage composition was determined in the bone marrow by using human-specific antibodies (see Table 5), and lineages were analyzed using the Attune N×T flow cytometer (ThermoFisher) and sorted using a FACSAria III cell sorter (BD Biosciences). CD34+ HSPCs or CD235a+ erythroblasts were isolated with magnetic beads, using the human-specific CD34 MicroBead Kit UltraPure (Miltenyi Biotec Inc., catalog #130-100-453) and CD235a (glycophorin A) MicroBeads, human, (Miltenyi Biotec Inc., catalog #130-050-501).

### Base Editing and Transplantation of Townes Mouse SCD HSPCs

Townes SCD mice were purchased from The Jackson Laboratory (Stock #013071). This strain harbors the human a-globin locus (HBA1) in place of the orthologous mouse loci Hba1 and Hba2, and the human g-globin (HBG1) and b-globin (HBB^{S} or HBBA) loci in place of the endogenous mouse loci Hbb-b1 and Hbb-b2. Bone marrow mononuclear cells were obtained by flushing femurs, tibias, hip bones and humeri with IMDM (10% FBS) followed by RBC lysis (ACK Lysing Buffer). Lineage marker negative (Lin−) cells enriched for HSPCs were purified by immuno-magnetic bead selection using the Mouse Lineage Cell Depletion Kit (Miltenyi, 130-090-858). Lin− cells were cultured in Lineage Negative Media: StemSpan SFEM supplemented with mSCF (100 ng/μL), mIL-3 (10 ng/μL), mIL-11 (100 ng/μL), hFLT3ligand (100 ng/μL) and PenStrep (1×) for 24 hours prior to base editing. Ribonucleoprotein complex was generated by incubating ABE8e-NRCH with targeting sgRNA at concentrations of 2.25 μM base editor and 6.75 uM gRNA (1:3 ratio) in T buffer (total volume 50 μL) for thirty minutes at room temperature. Electroporation was performed using the ThermoFisher Neon Transfection System with 100-μL tips in buffer E2 at 1700 pulse voltage, 20 pulse width, 1 pulse.

Following electroporation, cells were cultured overnight in Lineage Negative Media, followed by transplantation via tail-vein injection of 106 cells into lethally irradiated (1,125 cGy delivered as a single dose), 8-12 week old C57B1/6 PepBoy (CD45.1) recipients. For analysis following transplantation peripheral blood was collected from the retro-orbital sinus using heparinized micro-hematocrit capillary tubes. Complete blood counts CBCs were performed using a FORCYTE Veterinary Hematology Analyzer. CBC measurements were collected from untransplanted HBBA/S mice at 4-6 months of age. Blood smears were prepared using modified Romanowsky methanolic staining and eosin and thiazin methods. Engraftment was determined by flow cytometry for mouse anti-CD45.1-PE (BD Cat #553776) and mouse anti-CD45.2-FITC (BE Cat #561874).

### Oxygen Binding Measurements

Hemoglobin-oxygen equilibrium curves (OECs), to determine the oxygen binding affinity of HbA, HbS and HbG, were obtained using the Hemox Analyzer (TCS Scientific, New Hope, Pa.). Mouse blood or was added to the analysis buffer containing Hemox solution (pH 7.4 at 37° C.), Additive-A (BSA-20), and anti-foaming agent (AFA-25) according to the manufacturer's protocol. Each sample was oxygenated at 37° C. using compressed air and then deoxygenated with compressed N2, while subjected to continuous dual-wavelength spectrophotometry to determine the oxyhemoglobin to deoxyhemoglobin ratio along with continuous measurement of the oxygen partial pressure. OECs and p50 values were generated by the TCS Hemox Analysis Software.

### REFERENCES FOR EXAMPLE 5

1 Piel, F. B., Steinberg, M. H. & Rees, D. C. Sickle Cell Disease. N Engl J Med 377, 305, doi:10.1056/NEJMc1706325 (2017).
2 Richter, M. F. et al. Phage-assisted evolution of an adenine base editor with improved Cas domain compatibility and activity. Nat Biotechnol 38, 883-891, doi:10.1038/s41587-020-0453-z (2020).
3 Miller, S. M. et al. Continuous evolution of SpCas9 variants compatible with non-G PAMs. Nat Biotechnol 38, 471-481, doi:10.1038/s41587-020-0412-8 (2020).
4 Viprakasit, V., Wiriyasateinkul, A., Sattayasevana, B., Miles, K. L. & Laosombat, V. Hb G-Makassar [beta6(A3)Glu—>Ala; codon 6 (GAG—>GCG)]: molecular characterization, clinical, and hematological effects. Hemoglobin 26, 245-253, doi:10.1081/hem-120015028 (2002).
5 Sangkitporn, S., Rerkamnuaychoke, B., Sangkitporn, S., Mitrakul, C. & Sutivigit, Y. Hb G Makassar (beta 6:Glu-Ala) in a Thai family. J Med Assoc Thai 85, 577-582 (2002).
6 Nickel, R. S. & Kamani, N. R. Ethical Challenges in Hematopoietic Cell Transplantation for Sickle Cell Disease. Biol Blood Marrow Transplant 24, 219-227, doi:10.1016/j.bbmt.2017.08.034 (2018).
7 Kunz, J. B. & Kulozik, A. E. Gene Therapy of the Hemoglobinopathies. Hemasphere 4, e479, doi:10.1097/H59.0000000000000479 (2020).
8 Leonard, A., Tisdale, J. & Abraham, A. Curative options for sickle cell disease: haploidentical stem cell transplantation or gene therapy? Br J Haematol 189, 408-423, doi:10.1111/bjh.16437 (2020).
9 Magrin, E., Miccio, A. & Cavazzana, M. Lentiviral and genome-editing strategies for the treatment of beta-hemoglobinopathies. Blood 134, 1203-1213, doi:10.1182/blood.2019000949 (2019).
10 Demirci, S., Leonard, A., Haro-Mora, J. J., Uchida, N. & Tisdale, J. F. CRISPR/Cas9 for Sickle Cell Disease: Applications, Future Possibilities, and Challenges. Adv Exp Med Biol 1144, 37-52, doi:10.1007/5584_2018_331 (2019).
11 Davis, R., Gurumurthy, A., Hossain, M. A., Gunn, E. M. & Bungert, J. Engineering Globin Gene Expression. Mol Ther Methods Clin Dev 12, 102-110, doi:10.1016/j.omtm.2018.12.004 (2019).
12 Ribeil, J. A. et al. Gene Therapy in a Patient with Sickle Cell Disease. N Engl J Med 376, 848-855, doi:10.1056/NEJMoa1609677 (2017).
13 Esrick, E. B. et al. Post-Transcriptional Genetic Silencing of BCL11A to Treat Sickle Cell Disease. N Engl J Med, doi:10.1056/NEJMoa2029392 (2020).
14 Frangoul, H. et al. CRISPR-Cas9 Gene Editing for Sickle Cell Disease and beta-Thalassemia. N Engl J Med, doi:10.1056/NEJMoa2031054 (2020).
15 McIntosh, B. E. et al. Nonirradiated NOD,B6.SCID Il2rgamma−/− Kit(W41/W41) (NBSGW) mice support multilineage engraftment of human hematopoietic cells. Stem Cell Reports 4, 171-180, doi:10.1016/j.stemcr.2014.12.005 (2015).
16 Park, S. H. et al. Highly efficient editing of the beta-globin gene in patient-derived hematopoietic stem and progenitor cells to treat sickle cell disease. Nucleic Acids Res 47, 7955-7972, doi:10.1093/nar/gkz475 (2019).
17 Pattabhi, S. et al. In vivo Outcome of Homology-Directed Repair at the HBB Gene in HSC Using Alternative Donor Template Delivery Methods. Mol Ther Nucleic Acids 17, 277-288, doi:10.1016/j.omtn.2019.05.025 (2019).
18 Zuccaro, M. V. et al. Allele-Specific Chromosome Removal after Cas9 Cleavage in Human Embryos. Cell 183, 1650-1664 e1615, doi:10.1016/j.cell.2020.10.025 (2020).
19 Song, Y. et al. Large-Fragment Deletions Induced by Cas9 Cleavage while Not in the BEs System. Mol Ther Nucleic Acids 21, 523-526, doi:10.1016/j.omtn.2020.06.019 (2020).
20 Adikusuma, F. et al. Large deletions induced by Cas9 cleavage. Nature 560, E8-E9, doi:10.1038/s41586-018-0380-z (2018).
21 Kosicki, M., Tomberg, K. & Bradley, A. Repair of double-strand breaks induced by CRISPR-Cas9 leads to large deletions and complex rearrangements. Nat Biotechnol, doi:10.1038/nbt.4192 (2018).
22 Haapaniemi, E., Botla, S., Persson, J., Schmierer, B. & Taipale, J. CRISPR-Cas9 genome editing induces a p53-mediated DNA damage response. Nat Med 24, 927-930, doi:10.1038/s41591-018-0049-z (2018).
23 Enache, 0. M. et al. Cas9 activates the p53 pathway and selects for p53-inactivating mutations. Nat Genet 52, 662-668, doi:10.1038/s41588-020-0623-4 (2020).
24 Ihry, R. J. et al. p53 inhibits CRISPR-Cas9 engineering in human pluripotent stem cells. Nat Med 24, 939-946, doi:10.1038/s41591-018-0050-6 (2018).
25 Kurup, S. P., Moioffer, S. J., Pewe, L. L. & Harty, J. T. p53 Hinders CRISPR/Cas9-Mediated Targeted Gene Disruption in Memory CD8 T Cells In vivo. J Immunol 205, 2222-2230, doi:10.4049/jimmuno1.2000654 (2020).
26 Gaudelli, N. M. et al. Programmable base editing of A*T to G*C in genomic DNA without DNA cleavage. Nature 551, 464-471, doi:10.1038/nature24644 (2017).
27 Huang, T. P. et al. Circularly permuted and PAM-modified Cas9 variants broaden the targeting scope of base editors. Nat Biotechnol 37, 626-631, doi:10.1038/s41587-019-0134-y (2019).
28 Anzalone, A. V., Koblan, L. W. & Liu, D. R. Genome editing with CRISPR-Cas nucleases, base editors, transposases and prime editors. Nat Biotechnol 38, 824-844, doi:10.1038/s41587-020-0561-9 (2020).
29 Rees, H. A. & Liu, D. R. Base editing: precision chemistry on the genome and transcriptome of living cells. Nat Rev Genet 19, 770-788, doi:10.1038/s41576-018-0059-1 (2018).
30 Giardine, B. et al. Updates of the HbVar database of human hemoglobin variants and thalassemia mutations. Nucleic Acids Res 42, D1063-1069, doi:10.1093/nar/gkt911 (2014).
31 Hardison, R. C. et al. HbVar: A relational database of human hemoglobin variants and thalassemia mutations at the globin gene server. Hum Mutat 19, 225-233, doi:10.1002/humu.10044 (2002).
32 Wu, L. C. et al. Correction of sickle cell disease by homologous recombination in embryonic stem cells. Blood 108, 1183-1188, doi:10.1182/blood-2006-02-004812 (2006).
33 Hendel, A. et al. Chemically modified guide RNAs enhance CRISPR-Cas genome editing in human primary cells. Nat Biotechnol 33, 985-989, doi:10.1038/nbt.3290 (2015).
34 Traxler, E. A. et al. A genome-editing strategy to treat β-hemoglobinopathies that recapitulates a mutation associated with a benign genetic condition. Nature medicine 22, 987 (2016).
35 Hu, J. et al. Isolation and functional characterization of human erythroblasts at distinct stages: implications for understanding of normal and disordered erythropoiesis in vivo. Blood 121, 3246-3253, doi:10.1182/blood-2013-01-476390 (2013).
36 Hwang, G. H., Kim, J. S. & Bae, S. Web-Based CRISPR Toolkits: Cas-OFFinder, Cas-Designer, and Cas-Analyzer. Methods Mol Biol 2162, 23-33, doi:10.1007/978-1-0716-0687-2_2 (2021).
37 Bae, S., Park, J. & Kim, J. S. Cas-OFFinder: a fast and versatile algorithm that searches for potential off-target sites of Cas9 RNA-guided endonucleases. Bioinformatics 30, 1473-1475, doi:10.1093/bioinformatics/btu048 (2014).
38 Tsai, S. Q. et al. CIRCLE-seq: a highly sensitive in vitro screen for genome-wide CRISPR-Cas9 nuclease off-targets. Nat Methods 14, 607-614, doi:10.1038/nmeth.4278 (2017).
39 Lazzarotto, C. R. et al. Defining CRISPR-Cas9 genome-wide nuclease activities with CIRCLE-seq. Nat Protoc 13, 2615-2642, doi:10.1038/s41596-018-0055-0 (2018).
40 Kuscu, C., Arslan, S., Singh, R., Thorpe, J. & Adli, M. Genome-wide analysis reveals characteristics of off-target sites bound by the Cas9 endonuclease. Nat Biotechnol 32, 677-683, doi:10.1038/nbt.2916 (2014).
41 Tsai, S. Q. et al. GUIDE-seq enables genome-wide profiling of off-target cleavage by CRISPR-Cas nucleases. Nat Biotechnol 33, 187-197, doi:10.1038/nbt.3117 (2015).
42 Rees, H. A. et al. Improving the DNA specificity and applicability of base editing through protein engineering and protein delivery. Nat Commun 8, 15790, doi:10.1038/ncomms15790 (2017).
43 Zuris, J. A. et al. Cationic lipid-mediated delivery of proteins enables efficient protein-based genome editing in vitro and in vivo. Nat Biotechnol 33, 73-80, doi:10.1038/nbt.3081 (2015).
44 Luc, S. et al. Bc111a Deficiency Leads to Hematopoietic Stem Cell Defects with an Aging-like Phenotype. Cell Rep 16, 3181-3194, doi:10.1016/j.celrep.2016.08.064 (2016).
45 Harrington, D. J., Adachi, K. & Royer, W. E., Jr. The high resolution crystal structure of deoxyhemoglobin S. J Mol Biol 272, 398-407, doi:10.1006/jmbi.1997.1253 (1997).
46 Pagnier, J., Bihoreau, M. T., Baudin, V., Edelstein, S. J. & Poyart, C. [Polymerization and solubility of recombinant hemoglobins alpha 2 beta 2 6 Glu—>Ala (Hb Makassar) and alpha 2 beta 2 6 Glu—>A1a, 23 Val—>Ile]. C R Acad Sci III 316, 431-436 (1993).
47 Weber, L. et al. Editing a gamma-globin repressor binding site restores fetal hemoglobin synthesis and corrects the sickle cell disease phenotype. Sci Adv 6, doi:10.1126/sciadv.aay9392 (2020).
48 Demirci, S. et al. betaT87Q-Globin Gene Therapy Reduces Sickle Hemoglobin Production, Allowing for Ex vivo Anti-sickling Activity in Human Erythroid Cells. Mol Ther Methods Clin Dev 17, 912-921, doi:10.1016/j.omtm.2020.04.013 (2020).
49 Wu, Y. et al. Highly efficient therapeutic gene editing of human hematopoietic stem cells. Nat Med 25, 776-783, doi:10.1038/s41591-019-0401-y (2019).
50 Magnani, A. et al. Extensive multilineage analysis in patients with mixed chimerism after allogeneic transplantation for sickle cell disease: insight into hematopoiesis and engraftment thresholds for gene therapy. Haematologica 105, 1240-1247, doi:10.3324/haemato1.2019.227561 (2020).
51 Abraham, A. et al. Relationship between Mixed Donor-Recipient Chimerism and Disease Recurrence after Hematopoietic Cell Transplantation for Sickle Cell Disease. Biol Blood Marrow Transplant 23, 2178-2183, doi:10.1016/j.bbmt.2017.08.038 (2017).
52 Hacein-Bey-Abina, S. et al. LMO2-associated clonal T cell proliferation in two patients after gene therapy for SCID-X₁. Science 302, 415-419, doi:10.1126/science.1088547 (2003).
53 Howe, S. J. et al. Insertional mutagenesis combined with acquired somatic mutations causes leukemogenesis following gene therapy of SCID-X₁ patients. J Clin Invest 118, 3143-3150, doi:10.1172/JCI35798 (2008).
54 Stein, S. et al. Genomic instability and myelodysplasia with monosomy 7 consequent to EVI1 activation after gene therapy for chronic granulomatous disease. Nat Med 16, 198-204, doi:10.1038/nm.2088 (2010).
55 Shen, M. W. et al. Predictable and precise template-free CRISPR editing of pathogenic variants. Nature, doi:10.1038/s41586-018-0686-x (2018).
56 Webber, B. R. et al. Highly efficient multiplex human T cell engineering without double-strand breaks using Cas9 base editors. Nat Commun 10, 5222, doi:10.1038/s41467-019-13007-6 (2019).
57 Cheng, L. et al. Single-nucleotide-level mapping of DNA regulatory elements that control fetal hemoglobin expression. Nature Genetics (in revision) (2021).
58 Gaudelli, N. M. et al. Directed evolution of adenine base editors with increased activity and therapeutic application. Nat Biotechnol 38, 892-900, doi:10.1038/s41587-020-0491-6 (2020).
59 Grunewald, J. et al. CRISPR DNA base editors with reduced RNA off-target and self-editing activities. Nat Biotechnol 37, 1041-1048, doi:10.1038/s41587-019-0236-6 (2019).
60 Yu, Y. et al. Cytosine base editors with minimized unguided DNA and RNA off-target events and high on-target activity. Nat Commun 11, 2052, doi:10.1038/s41467-020-15887-5 (2020).
61 Doman, J. L., Raguram, A., Newby, G. A. & Liu, D. R. Evaluation and minimization of Cas9-independent off-target DNA editing by cytosine base editors. Nat Biotechnol 38, 620-628, doi:10.1038/s41587-020-0414-6 (2020).
62 Kumar, S. & Geiger, H. HSC Niche Biology and HSC Expansion Ex vivo. Trends Mol Med 23, 799-819, doi:10.1016/j.molmed.2017.07.003 (2017).
63 Li, C. et al. Reactivation of gamma-globin in adult beta-YAC mice after ex vivo and in vivo hematopoietic stem cell genome editing. Blood 131, 2915-2928, doi:10.1182/blood-2018-03-838540 (2018).
64 Levy, J. M. et al. Cytosine and adenine base editing of the brain, liver, retina, heart and skeletal muscle of mice via adeno-associated viruses. Nat Biomed Eng 4, 97-110, doi:10.1038/s41551-019-0501-5 (2020).
65 Chatterjee, S., Sivanandam, V. & Wong Kk, Jr. Adeno-Associated Virus and Hematopoietic Stem Cells: The Potential of Adeno-Associated Virus Hematopoietic Stem Cells in Genetic Medicines. Hum Gene Ther 31, 542-552, doi:10.1089/hum.2020.049 (2020).
66 Koblan, L. W. et al. In vivo Adenine Base Editing Corrects Hutchinson-Gilford Progeria Syndrome. Nature In press. (2020).
67 Komor, A. C., Kim, Y. B., Packer, M. S., Zuris, J. A. & Liu, D. R. Programmable editing of a target base in genomic DNA without double-stranded DNA cleavage. Nature 533, 420-424, doi:10.1038/nature17946 (2016).
68 Clement, K. et al. CRISPResso2 provides accurate and rapid genome editing sequence analysis. Nat Biotechnol 37, 224-226, doi:10.1038/s41587-019-0032-3 (2019).
69 Vaidyanathan, S. et al. Uridine Depletion and Chemical Modification Increase Cas9 mRNA Activity and Reduce Immunogenicity without HPLC Purification. Mol Ther Nucleic Acids 12, 530-542, doi:10.1016/j.omtn.2018.06.010 (2018).
70 Connelly, J. P. & Pruett-Miller, S. M. CRIS.py: A Versatile and High-throughput Analysis Program for CRISPR-based Genome Editing. Sci Rep 9, 4194, doi:10.1038/s41598-019-40896-w (2019).
71 Heinz, S. et al. Simple combinations of lineage-determining transcription factors prime cis-regulatory elements required for macrophage and B cell identities. Mol Cell 38, 576-589, doi:10.1016/j.molce1.2010.05.004 (2010).
72 Leonard, A. et al. Low-Dose Busulfan Reduces Human CD34(+) Cell Doses Required for Engraftment in c-kit Mutant Immunodeficient Mice. Mol Ther Methods Clin Dev 15, 430-437, doi:10.1016/j.omtm.2019.10.017 (2019).

### Other Embodiments and Equivalents

The foregoing has been a description of certain non-limiting embodiments of the disclosure. Those of ordinary skill in the art will appreciate that various changes and modifications to this description may be made without departing from the spirit or scope of the present disclosure, as defined in the following claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the disclosure shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. An adenosine deaminase that comprises the substitutions selected from T111X₁, D119X₂, and F149X₃, and further comprises at least one substitution selected from R26X₄, V88X₅, A109X₆, H122X₇, T166X₈, and D167X₉, relative to the amino acid sequence of SEQ ID NO: 315, or a corresponding substitution(s) in another adenosine deaminase, wherein X₁ is any amino acid other than T, X₂ is any amino acid other than D, X₃ is any amino acid other than F, X₄ is any amino acid other than R, X₅ is any amino acid other than V, X₆ is any amino acid other than A, X₇ is any amino acid other than H, X₈ is any amino acid other than T, and X₉ is any amino acid other than D.

2. The adenosine deaminase of claim 1, wherein X₁ is R, X₂ is D, and X₃ is Y.

3. The adenosine deaminase of claim 1, wherein X₆ is 5, X₇ is N, X₈ is I, and X₉ is N.

4. The adenosine deaminase of claim 1, wherein X₄ is C and X₅ is A.

5. The adenosine deaminase of claim 1, further comprising a Y167X₁₀ relative to the amino acid sequence of SEQ ID NO: 315, or a corresponding substitution in another adenosine deaminase, wherein X₁₀ is any amino acid other than Y.

6-8. (canceled)

9. An adenosine deaminase that comprises T111R, D119N, and F149Y substitutions, and further comprises at least one substitution selected from R26C, V88A, A109S, H122N, T1661, and D167N, relative to the amino acid sequence of SEQ ID NO: 315, or corresponding substitutions in another adenosine deaminase.

10. The adenosine deaminase of claim 9, wherein the adenosine deaminase comprises A109S, T111R, D119N, H122N, F149Y, T1661, and D167N substitutions in SEQ ID NO: 315.

11. The adenosine deaminase of claim 9, wherein the adenosine deaminase comprises T111R, D119N, and F149Y substitutions and an R26C or V88A substitution in SEQ ID NO: 315.

12. The adenosine deaminase of claim 9, further comprising a Y147D substitution.

13. The adenosine deaminase of claim 9, wherein the adenosine deaminase comprises A109S, T111R, D119N, H122N, Y147D, F149Y, T1661, and D167N substitutions in SEQ ID NO: 315.

14. The adenosine deaminase of claim 9, further comprising at least one substitution in the amino acid sequence of SEQ ID NO: 315 selected from K20A, R21A, V82G, and V106W.

15. The adenosine deaminase of claim 9, wherein the adenosine deaminase comprises V106W, A109S, T111R, D119N, H122N, Y147D, F149Y, T1661, and D167N substitutions in SEQ ID NO: 315.

16. The adenosine deaminase of claim 9, wherein the adenosine deaminase comprises the following substitutions relative to the amino acid sequence of SEQ ID NO: 325: W23R, H36L, P48A, R51L, L84F, A106V, D108N, A109S, T111R, D119N, H122N, H123Y, S146C, F149Y, R152P, E155V, I156F, K157N, T1661, and D167N.

17. The adenosine deaminase of claim 9, wherein the adenosine deaminase comprises the following substitutions relative to the amino acid sequence of SEQ ID NO: 325: W23R, H36L, P48A, R51L, L84F, A106W, D108N, A109S, T111R, D119N, H122N, H123Y, S146C, F149Y, R152P, E155V, I156F, K157N, T1661, and D167N.

18-55. (canceled)

56. A base editor comprising an amino acid sequence that is at least 80% identical to the amino acid sequence of any one of SEQ ID NOs: 169-188, 449-462, and 465-476.

57-63. (canceled)

64. The base editor of claim 56, wherein the base editor generates less than 20% indel formation when contacted with a nucleic acid comprising a target sequence.

65. The base editor of claim 56, wherein the base editor provides an efficiency of conversion of an adenine (A) base to a guanine (G) base of at least 70when contacted with a DNA comprising a target sequence selected from the group consisting of AAA, AAT, AAC, AAG, TAA, TAT, TAC, TAG, CAA, CAT, CAC, CAG, GAA, GAT, GAC, and GAG.

66-68. (canceled)

69. The base editor of claim 56, wherein the base editor causes an off-target DNA editing frequency of less than 1.5% when contacted with a DNA comprising a target sequence.

70-71. (canceled)

72. The base editor of claim 56, wherein the base editor provides an adenosine (A) to inosine (I) editing frequency in RNA of 0.3% or less when contacted with a DNA comprising a target sequence.

73-75. (canceled)

76. The base editor of claim 56, wherein the base editor is selected from ABE8e, ABE8e(TadA-8e V106W), CP1028-ABE8e, CP1041-ABE8e, and ABE8e-NRCH.

77-188. (canceled)
